(19) United States
(12) Patent Application Publication
(10) Pub. No.: US 2011/0098296 A1
(43) Pub. Date: Apr. 28, 2011

(54) Thiazole And Oxazole Kinase Inhibitors

(76) Inventors: George Adjabeng, Durham, NC (US); Neil Bifulco, Durham, NC (US); Ronda G. Davis-Ward, Durham, NC (US); Scott Howard Dickerson, Durham, NC (US); Keith Hornberger, Durham, NC (US); Kimberly Petrov, Durham, NC (US); Tara Renae Rheault, Durham, NC (US); Daivd Edward Uehling, Durham, NC (US); Alex Gregory Waterson, Durham, NC (US)

(21) Appl. No.: 12/746,826

(22) PCT Filed: Dec. 4, 2008
(86) PCT No.: PCT/US08/85452

§ 371 Date: Jun. 8, 2010

Related U.S. Application Data
(60) Provisional application No. 61/013,409, filed on Dec. 13, 2007.

(51) Int. Cl. A61K 31/5377; C07D 413/14; C07D 401/04; A61K 31/497; A61K 31/506; A61P 35/00

(52) U.S. Cl. 514/235.2; 544/122; 544/331; 514/235.8; 514/252.19; 514/275

(57) ABSTRACT

The present invention provides thiazole and oxazole compounds, compositions containing the same, as well as processes for the preparation and methods for their use as pharmaceutical agents.

### FIELD OF THE INVENTION

The present invention relates to thiazole and oxazole compounds, compositions containing the same, as well as processes for the preparation and methods of using such compounds and compositions.

### BACKGROUND OF THE INVENTION

Both receptor tyrosine kinases and serine/threonine kinases have been implicated in cellular signaling pathways that control cell function, division, growth, differentiation, and death (apoptosis) through reversible phosphorylation of the hydroxyl groups of tyrosine or serine and threonine residues, respectively, in proteins. In signal transduction, for example, extracellular signals are transduced via membrane receptor activation, with amplification and propagation using a complex choreography of cascades of protein phosphorylation, and protein dephosphorylation events to avoid uncontrolled signaling. These signaling pathways are highly regulated, often by complex and intermeshed kinase pathways where each kinase may itself be regulated by one or more other kinases and protein phosphatases. The biological importance of these finely tuned systems is such that a variety of cell proliferative disorders have been linked to defects in one or more of the various cell signaling pathways mediated by tyrosine or serine/threonine kinases.

Receptor tyrosine kinases (RTKs) catalyze phosphorylation of certain tyrosyl amino acid residues in various proteins, including themselves, which govern cell growth, proliferation and differentiation. The ErbB family of RTKs includes EGFR, ErbB2, ErbB3, and ErbB4. Aberrant activity in the ErbB family kinases has been implicated in a range of hyperproliferative disorders including psoriasis, rheumatoid arthritis, bronchitis, and several cancers. The biological role of ErbB family RTKs and their implication in different disease states is discussed, for instance, in Ullrich, A., et al., Cell (Apr. 20, 1990) 61: 203-212; Aaronson, S., Science (1991) 254:1146-1153; Salomon, D., et al., Crit. Rev. Oncol./Hematol. (1995) 19:183-232; Woodburn, J. R., Pharmacol. Ther. (1999) 82: 2-3, 241-250; Normanno, N., et al., Curr. Drug Targets (2005) 6:243-257; and Hynes, N. et al., Nat. Rev. Cancer (2005) 5:341-345. In particular, elevated EGFR activity has been implicated in non-small cell lung, squamous cell lung, breast, bladder, head and neck squamous cell, esophageal, gastric, colorectal, pancreatic, thyroid, glial, cervical, and ovarian cancers (Salomon (1995) supra; Woodburn (1999) supra; Normanno (2005) supra; Hynes (2005) supra) and hepatocellular cancer Thomas, M. B., et al., Cancer (2007) 110(5):1059-1067 and Huether A., et al., J. Hepatol. (2005) 43(4):661-669. Furthermore, overexpression and/or mutation of ErbB2 has been implicated in the following:
Barret's adenocarcinoma (Brian et al., Hum. Pathol. (2000) 31 (1) 35-39);
bladder cancer (Simon et al., Int. J. Cancer (2003) 107 (5) 764-772);
breast cancer (Salmon et al., Science (1987) 235 (4785) 177-182);
central nervous system tumors including primary CNS tumors such as glioblastomas, astrocytomas, ependymomas and glioblastoma multiforme (Potti et al., Cancer Invest. (2004) 22 (4) 537-544; Koka et al., Am. J. Clin. Oncol. (2003) 26 (4) 332-335; and Haynik et al., Appl. Immunohistochem. Mol. Morphol. (2007) 15 (1) 56-68) and secondary CNS tumors (i.e., metastases to the central nervous system of tumors originating outside of the central nervous system),
gastric cancer including esophageal cancer, gastro-esophageal cancer, and salivary gland mucoepidermoid carcinoma (Tanner et al., Ann. Oncol. (2005) 16 (2) 273-278; Yano et al., Oncol. Rep. (2006) 15 (1) 65-71; Mimura et al., Br. J. Cancer (2005) 92 (7) 1253-1260; and Nguyen et al., J. Otolaryngol. (2003) 32 (5) 328-331);
carcinoma of the head and neck including squamous cell carcinoma of the head and neck (Weed et al., Head Neck (2004) 26 (4) 353-364);
lung cancer (Stephens et al., Nature (2004) 431 525-526), including small cell lung cancer (Canoz et al., Lung (2006) 184 (5) 267-272) and non-small cell lung cancer (Hirsch et al., Br. J. Cancer (2002) 86 (9) 1449-1456); melanoma (Nyormoi & Bar-Eli Clin. Exp. Metastasis (2003) 20 (3) 251-263);
ovarian cancer (Slamon et al., Science (1989) 244 (4905) 707-712) and endometrial cancer (Morrison et al., J. Clin. Oncol. (2006) 24 (15) 2376-2385 and Liu Taiwan. J. Obstet. Gynecol. (2007) 46 (1) 26-32);
pancreatic endocrine tumors (Goebel et al., Cancer Res. (2002) 62 (13) 3702-3710);
renal cancer (Latif et al., BJU Int. (2002) 89 (1) 5-9) and Wilm's tumor (Menard et al., Cell. Mol. Life Sci. (2004) 61 (23) 2965-2978); and
   uterine cancer including uterine sarcoma (Slomovitz et al., J. Clin. Oncol. (2004) 22 (15) 3126-3132; and Amant et al., Gynecol. Oncol. (2004) 95 (3) 583-587).

A timeline of events pertaining to the role of the ErbB family kinases in cancer may be found in Gschwind. A., et al., Nat. Rev. Cancer (2004) 4:361-370. By virtue of the role played by the ErbB family kinases in these cancers and the relative success of inhibitors of these kinases in the clinic, it is widely acknowledged that inhibitors of one or more ErbB family kinases will be useful for the treatment of such cancers.

Downstream of the several RTKs, including EGFR and ErbB2, lie several signaling pathways, among them being the Ras-Raf-MEK-ERK kinase pathway. It is currently understood that activation of Ras GTPase proteins or other upstream kinases in response to growth factors, hormones, cytokines, etc. stimulates phosphorylation and activation of Raf kinases. These kinases then phosphorylate and activate the intracellular protein kinases MEK1 and MEK2, which in turn phosphorylate and activate other protein kinases, ERK1 and ERK2. This signaling pathway, also known as the mitogen-activated protein kinase (MAPK) pathway or cytoplasmic cascade, mediates cellular responses to growth signals. The ultimate function of this is to link receptor activity at the cell membrane with modification of other membrane-associated, cytoplasmic, or nuclear targets that govern cell proliferation, differentiation, and survival. Mutations in various Ras GTPases and the B-Raf kinase have been identified that can lead to sustained and constitutive activation of the MAPK pathway, ultimately resulting in increased cell division and survival. As a consequence of this, these mutations have been strongly linked with the establishment, development, and progression of a wide range of human cancers. The biological role of the Raf kinases, and specifically that of B-Raf, in signal transduction is described in Davies, H., et al., Nature (2002) 9:1-6; Garnett, M. J. & Marais, R., Cancer Cell (2004) 6:313-319; Zebisch, A. & Troppmair, J., Cell. Mol. Life. Sci. (2006) 63:1314-1330; Midgley, R. S. & Kerr, D. J., Crit. Rev. Onc/Hematol. (2002) 44:109-120; Smith, R. A., et al., Curr. Top. Med. Chem. (2006) 6:1071-1089; and Downward, J., Nat. Rev. Cancer (2003) 3:11-22.

Naturally occurring mutations of the B-Raf kinase that activate MAPK pathway signaling have been found in a large percentage of human melanomas (Davies (2002) supra) and thyroid cancers (Cohen et al., J. Nat. Cancer Inst. (2003) 95 (8) 625-627 and Kimura et al., Cancer Res. (2003) 63 (7) 1454-1457), as well as at lower, but still significant, frequencies in the following:
Barret's adenocarcinoma (Garnett et al., Cancer Cell (2004) δ 313-319 and Sommerer et al., Oncogene (2004) 23 (2) 554-558),
billiary tract carcinomas (Zebisch et al., Cell. Mol. Life. Sci. (2006) 63 1314-1330), breast cancer (Davies (2002) supra),
cervical cancer (Moreno-Bueno et al., Clin. Cancer Res. (2006) 12 (12) 3865-3866),
cholangiocarcinoma (Tannapfel et al., Gut (2003) 52 (5) 706-712),
central nervous system tumors including primary CNS tumors such as glioblastomas, astrocytomas and ependymomas (Knobbe et al., Acta Neuropathol. (Berl.) (2004) 108 (6) 467-470, Davies (2002) supra, and Garnett et al., Cancer Cell (2004) supra) and secondary CNS tumors (i.e., metastases to the central nervous system of tumors originating outside of the central nervous system),
colorectal cancer, including large intestinal colon carcinoma (Yuen et al., Cancer Res. (2002) 62 (22) 6451-6455, Davies (2002) supra and Zebisch et al., Cell. Mol. Life. Sci. (2006),
gastric cancer (Lee et al., Oncogene (2003) 22 (44) 6942-6945),
carcinoma of the head and neck including squamous cell carcinoma of the head and neck (Cohen et al., J. Nat. Cancer Inst. (2003) 95 (8) 625-627 and Weber et al., Oncogene (2003) 22 (30) 4757-4759),
hematologic cancers including leukemias (Garnett et al., Cancer Cell (2004) supra, particularly acute lymphoblastic leukemia (Garnett et al., Cancer Cell (2004) supra and Gustafsson et al., Leukemia (2005) 19 (2) 310-312), acute myelogenous leukemia (AML) (Lee et al., Leukemia (2004) 18 (1) 170-172, and Christiansen et al., Leukemia (2005) 19 (12) 2232-2240), myelodysplastic syndromes (Christiansen et al., Leukemia (2005) supra) and chronic myelogenous leukemia (Mizuchi et al., Biochem. Biophys. Res. Commun. (2005) 326 (3) 645-651); Hodgkin's lymphoma (Figl et al., Arch. Dermatol. (2007) 143 (4) 495-499), non-Hodgkin's lymphoma (Lee et al., Br. J. Cancer (2003) 89 (10) 1958-1960), megakaryoblastic leukemia (Eychene et al., Oncogene (1995) 10 (6) 1159-1165) and multiple myeloma (Ng et al., Br. J. Haematol. (2003) 123 (4) 637-645),
hepatocellular carcinoma (Garnett et al., Cancer Cell (2004),
lung cancer (Brose et al., Cancer Res. (2002) 62 (23) 6997-7000, Cohen et al., J. Nat. Cancer Inst. (2003) supra and Davies (2002) supra), including small cell lung cancer (Pardo et al., EMBO J. (2006) 25 (13) 3078-3088) and non-small cell lung cancer (Davies (2002) supra),
ovarian cancer (Russell & McCluggage J. Pathol. (2004) 203 (2) 617-619 and Davies (2002) supra), endometrial cancer (Garnett et al., Cancer Cell (2004) supra, and Moreno-Bueno et al., Clin. Cancer Res. (2006) supra),
pancreatic cancer (Ishimura et al., Cancer Lett. (2003) 199 (2) 169-173),
pituitary adenoma (De Martino et al., J. Endocrinol. Invest. (2007) 30 (1) RC1-3),
prostate cancer (Cho et al., Int. J. Cancer (2006) 119 (8) 1858-1862),
renal cancer (Nagy et al., Int. J. Cancer (2003) 106 (6) 980-981),
sarcoma (Davies (2002) supra), and
skin cancers (Rodriguez-Viciana et al., Science (2006) 311(5765) 1287-1290 and Davies (2002) supra).

Overexpression of c-Raf has been linked to AML (Zebisch et al., Cancer Res. (2006) 66 (7) 3401-3408, and Zebisch (Cell. Mol. Life Sci. (2006)) and erythroleukemia (Zebisch et la., Cell. Mol. Life. Sci. (2006).

By virtue of the role played by the Raf family kinases in these cancers and exploratory studies with a range of preclinical and therapeutic agents, including one specifically targeted to inhibition of B-Raf kinase activity (King A. J., et al., (2006) Cancer Res. 66:11100-11105), it is generally accepted that inhibitors of one or more Raf family kinases will be useful for the treatment of such cancers.

PCT Publication No. WO2003/029249, published 10 Apr. 2003 to Syngenta, recites fungicidal compounds of the formula:

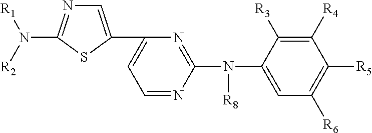

wherein
R₁ is H, C₁-C₆alkyl, C₂-C₆alkenyl, C₃-C₇cycloalkyl, C₃-C₇cycloalkyl-C₁-C₄alkyl, C₁-C₄alkyl-C₃-C₇cycloalkyl-C₁-C₄alkyl, C₁-C₆haloalkyl, C₁-C₆hydroxalkyl, C₁-C₄alkoxy-C₁-C₆alkyl, C₁-C₆-aminoalkyl, C₁-C₄alkyl-C₁-C₆-aminoalkyl, di(C₁-C₄alkyl)-C₁-C₆-aminoalkyl, aryl-C₁-C₄alkyl, heteroaryl-C₁-C₄alkyl, or a group —CO—R₉, —CO—OR₁₀, —CO—NR₁₀R₁₁, or —NR₁₀R₁₁,
R₂ is H, C₁-C₆alkyl, C₁-C₆haloalkyl, C₁-C₆hydroxalkyl, C₁-C₄alkoxy-C₁-C₆alkyl, C₁-C₆-aminoalkyl, C₁-C₄alkyl-C₁-C₆-aminoalkyl or a group —CO—R₉;
R₁ and R₂ together with the nitrogen to which they are bound form an optionally substituted N-linked saturated or unsaturated N-ring system which may contain oxygen or sulfur as a ring member, or form a group —N═CR₉—NR₁₀R₁₁;
R₃ is H, halogen or C₁-C₄alkyl;
R₄ is H, C₁-C₆alkyl, C₁-C₆haloalkyl, C₁-C₆cyanoalkyl, C₃-C₇cycloalkyl, C₂-C₆alkenyl, C₂-C₆haloalkenyl, C₂-C₆alkynyl, C₂-C₆haloalkynyl, amino, C₁-C₆-aminoalkyl, di(C₁-C₄alkyl)-amino, halogen, hydroxy, mercapto, cyano, C₁-C₆alkoxy, C₂-C₆alkenyloxy, C₂-C₆alkynyloxy, C₁-C₆haloalkoxy, C₁-C₆alkanoyloxy-C₁-C₆alkyl, C₁-C₆alkylthio, C₁-C₆alkylsulfinyl, C₁-C₆alkylsulfonyl, C₁-C₆hydroxalkyl, C₁-C₄alkoxy-C₁-C₆alkyl, C₁-C₆-aminoalkyl, C₁-C₄alkyl-C₁-C₆aminoalkyl, di(C₁-C₄alkyl)-C₁-C₈-aminoalkyl, C₁-C₈alkoxycarbonyl, C₁-C₈alkanoyl-C₁-C₆aminoalkyl, optionally substituted heterocyclyl, optionally substituted aryl, optionally substituted heteroaryl, or a group —CO—R₉, —O—CO—R₉, —NH—CO—R₉, —(C₁-C₆alkylene)-CO—R₉, —C(—O—C₁-C₆alkylene-O—)—R₉, —C(═NOR₈)—R₉ or —CO—NR₁₀R₁₁;
R₅ is H, hydroxy, halogen, C₁-C₈alkyl, C₁-C₆alkoxy or C₁-C₆haloalkyl;
R₆ is H, C₁-C₆alkyl or C₁-C₆haloalkyl;
R₇ is thienyl, pyridinyl or aryl each optionally substituted with one to three substituents independently selected from the group comprising halogen, C₁-C₄alkyl, C₁-C₄alkoxy, C₁-C₄haloalkyl and C₁-C₄haloalkoxy;
R₈ is H, C₁-C₆alkyl, C₂-C₆alkenyl, C₂-C₆alkynyl, C₁-C₄alkoxy-C₁-C₆alkyl, or a group —CO—R₉ or —CO—OR₁₀;
R₉ is H, C₁-C₆alkyl, C₁-C₈haloalkyl, C₃-C₇cycloalkyl, C₃-C₇cycloalkyl-C₁-C₄alkyl, aryl, C₁-C₄alkyl-C₃-C₇cycloalkyl-C₁-C₄alkyl, aryl-C₁-C₄alkyl, heteroaryl or heteroaryl-C₁-C₄alkyl;
R₁₀ is C₁-C₆alkyl, C₁-C₈haloalkyl, C₃-C₇cycloalkyl-C₁-C₄alkyl or C₁-C₄alkoxy-C₁-C₆alkyl;
R₁₁ is C₁-C₆alkyl, C₃-C₇cycloalkyl, C₃-C₇cycloalkyl-C₁-C₄alkyl, C₁-C₄alkoxy-C₁-C₆alkyl aryl or heteroaryl; or a salt thereof.

PCT publication No. WO02/043467, published 27 May 2004 to Cyclacel Ltd., recites antiviral compounds of the formula

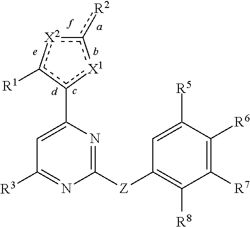

wherein
(A) one of X¹ and X² is S, and the other of X¹ and X² is N;
“a” is a single bond; and
“b”, “c”, “d”, “e” and “f” are single or double bonds so as to form a thiazolyl ring;
R² is independently as defined below for R¹ and R³; or
   (B) one of X¹ and X² is S, and the other of X¹ and X² is NR⁹;
“a” and “d” are each double bonds; and
“b”, “c”, “e” and “f” are each single bonds;
R² is oxo;
R⁹ is H or alkyl;
   where:

### Z is NH, NHCO, NHSO₂, NHCH₂, CH₂, CH₂CH₂, or CH═CH;

R¹ and R³ are independently H, alkyl, aryl, arylalkyl, heterocycle, halogen, NO₂, CN, OH, alkoxy, aryloxy, NH₂, NH-alkyl, N—(R′)(R″), NH-aryl, N-(aryl)₂, NHCOR′, COOH, COO-alkyl, COO-aryl, CONH₂, CONH—R′, CON—(R′)(R″), CONH-aryl, CON-(aryl)₂, SO₃H, SO₂NH₂, CF₃, CO—R′, or CO-aryl, wherein said alkyl, NH-aryl, COO-alkyl, NH-alkyl, aryl, arylalkyl and heterocycle groups may be further substituted with one or more groups selected from halogen, NO₂, CN, OH, O-methyl, NH₂, COOH, N—(R′)(R″), CONH₂ and CF₃;
R⁴, R⁵, R⁶, R⁷ and R⁸ are independently from each other H, optionally substituted lower alkyl, halogen, NO₂, CN, OH, substituted or unsubstituted alkoxy, NH₂, NH—R′, alkyl-aryl, alkyl-heteroaryl, NH(C═NH)NH₂, N(R′)₃⁺, N—(R′)(R″), COOH, COO—R′, CONH₂, CONH—R′, CON—(R′)(R″), SO₃H, SO₂NH₂, CF₃, or (CH₂)ₙO(CH₂)ₘNR′R″, (CH₂)ₙCO₂(CH₂)ₘOR′″ wherein n is 0, 1, 2 or 3 and m is 1, 2 or 3;
wherein R′ and R″ are each independently substituted or unsubstituted alkyl or alkenyl groups that may be the same or different;
   and pharmaceutically acceptable salts thereof.

See also, PCT Publication Nos. WO2004/043953, published 27 May 2004 and WO2005/116025, published 8 Dec. 2005, also to Cyclacel Lmtd.

PCT Publication No. 2004/056368, published 8 Jul. 2004 to Cyclacel Lmtd., recites compounds for treating diabetes and CNS disorders, alopecia, CV disorders and stroke, having the formula:

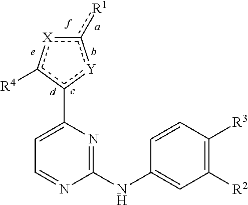

wherein
(A) one of X and Y is S, and the other is N; or
one of X and Y is NH or N—R⁵, and the other is C—R⁶;
“a” is a single bond; and
“b”, “c”, “d”, “e” and “f” are single or double bonds so as to form a heteroaryl ring;
R¹ is R⁷ with the proviso that R¹ is other than H or Me; or
   (B) one of X and Y is S, and the other is NH or N—R⁵;
“a” and “d” are each double bonds; and
“b”, “c”, “e” and “f” are each single bonds;
R¹ is oxo; and
   R², R³, R⁴, R⁵, and R⁶ are each independently H or R⁷;
R⁷ is a group (CH₂)ₙ—R⁸, wherein n is 0, 1, 2, 3 or 4 and wherein R⁸ is selected from alkyl, aryl, heteroaryl, heterocycloalkyl, F, Cl, Br, I, CF₃, NO₂, CN, OH, O-alkyl, O-aryl, O-heteroaryl, O-heterocycloalkyl, CO-alkyl, CO-aryl, CO-heteroaryl, CO-heterocycloalkyl, COO-alkyl, NH₂, NH-alkyl, NH-aryl, N(alkyl)₂, NH-heteroaryl, NH-heterocycloalkyl, COOH, CONH₂, CONH-alkyl, CON(alkyl)₂, CONH-aryl, CONH-heteroaryl, CONH-heterocycloalkyl, SO₃H, SO₂-alkyl, SO₂-aryl, SO₂-heteroaryl, SO₂-heterocycloalkyl, SO₂NH₂, SO₂NH-alkyl, SO₂N(alkyl)₂, SO₂NH-aryl, SO₂NH-heteroaryl, SO₂NH-heterocycloalkyl, wherein said alkyl, aryl, heteroaryl, and heterocycloalkyl groups are optionally substituted with one or more groups selected from halogen, NO₂, OH, O-methyl, NH₂, COOH, CONH₂ and CF₃.

### SUMMARY OF THE INVENTION

In a first aspect of the present invention, there is provided compounds of formula (I):

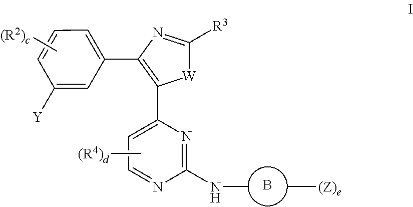

wherein:
Y is a moiety selected from i, ii, and iii:

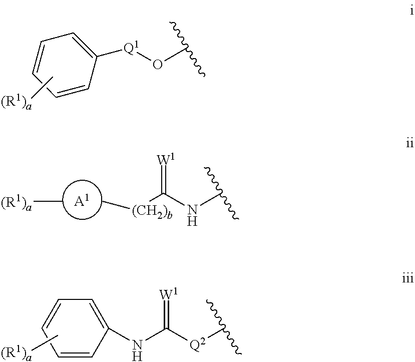

wherein:
a is 0, 1, 2 or 3;
each R¹ is the same or different and is independently selected from halo, alkyl, haloalkyl, —OR⁶, —R⁵—OR⁶, —C(O)R⁶, —CO₂R⁶, —S(O)_{f}R⁶, —R⁵—S(O)_{f}R⁶, —NR⁶R⁷, —R⁵—NR⁶R⁷, —CN and —R⁵—CN;
f is 0, 1 or 2;
Q¹ is —CH₂— or —SO₂—;
Ring A¹ is cycloalkyl, phenyl or 5-10 membered heteroaryl having 1 or 2 heteroatoms selected from N, O and S;
b is 0 or 1;
W¹ is O or S;
Q² is a bond or —N(H)—;
   c is 0, 1 or 2;
each R² is the same or different and is independently selected from halo, alkyl, haloalkyl, —OR⁶, —S(O)_{f}R⁶, —NR⁶R⁷, —CN and —NO₂;

### W is —O— or —S—;

R³ is selected from H, alkyl, haloalkyl, alkenyl, cycloalkyl, —R⁵-cycloalkyl, Ph, Het, —R⁵—OR⁶, —R⁵—S(O)_{f}R⁶, —R⁵—S(O)₂—NR⁶R⁷, —NR⁶R⁷, —N(R⁶)-cycloalkyl, —N(R⁶)Ph, —N(R⁶)Het, —N(R⁶)R⁵—Het, —N(R⁶)—R⁵—OR⁷, —N(R⁶)—R⁵—NR⁶R⁷, —N(H)C(O)R⁶, —R⁵—N(H)C(O)R⁶, —N(R⁶)—C(O)—NR⁶R⁷, —N(H)SO₂R⁶, —N(R⁶)—R⁵—S(O)_{f}R⁷, and —N(R⁶)—S(O)₂—NR⁶R⁷,
   wherein each of said cycloalkyl is optionally substituted with 1 or 2 substituents which are the same or different and are each independently selected from halo, C₁₋₃alkyl, haloalkyl, OH, oxo, S(C₁₋₃alkyl), SO₂, NH₂, N(H)C₁₋₃alkyl, and N(C₁₋₃alkyl)₂;
      d is 0, 1 or 2;
each R⁴ is the same or different and is independently selected from halo, alkyl, haloalkyl, —S(O)_{f}R⁶, —NR⁶R⁷, —CN and —NO₂;
each Ph is the same or different and is independently phenyl optionally substituted with 1, 2 or 3 substituents which are the same or different and are each independently selected from halo, C₁₋₃alkyl, haloC₁₋₃alkyl, O—C₁₋₃alkyl, C₁₋₃alkylene-O—C₁₋₃alkyl, OH, C₁₋₃alkylene-OH, NH₂, N(H)C₁₋₃alkyl, N(C₁₋₃alkyl)₂, CN and NO₂;
each Het is the same or different and is independently selected from 4-6 membered heterocycle having 1 or 2 heteroatoms selected from N, O and S and optionally substituted with 1, 2 or 3 substituents which are the same or different and are each independently selected from halo, C₁₋₃alkyl, O—C₁₋₃alkyl, C₁₋₃alkylene-O—C₁₋₃alkyl, OH, C₁₋₃alkylene-OH, oxo, C(O)(C₁₋₃alkyl), C(O)NH₂, C(O)N(C₁₋₃alkyl)₂, SO₃(H), SO₂(C₁₋₃alkyl), C₁₋₃alkylene-SO₃(H), C₁₋₃alkylene-SO₂(C₁₋₃alkyl), NH₂, N(H)C₁₋₃alkyl, N(C₁₋₃alkyl)₂, CN, —CH₂CN, and NO₂;
   each R⁵ is the same or different and is independently C₁₋₄alkylene;
Ring B is selected from phenyl, 9-10 membered aryl, 5-6 membered heteroaryl, and 9-10 membered heteroaryl, each heteroaryl having 1, 2 or 3 heteroatoms selected from N, O and S;
   wherein when Ring B is selected from phenyl and 5-6 membered heteroaryl, then e is 0, 1, 2 or 3; and
   each Z is the same or different and is independently selected from:
      halo, alkyl, haloalkyl, alkenyl,
      Het², —R⁵Het², Het³-Het²,
      oxo, —OR⁶, —R⁵—OR⁶, —O—R⁵—OR⁶, —OHet², —O—R⁵—Het², —O—R⁵—NR⁶R⁷,
      —O—R⁵—S(O)₂R⁶, —C(O)NR⁶R⁷, —R⁵—C(O)NR⁶R⁷, —CO₂R⁶, —R⁶—CO₂R⁶,
      —S(O)_{f}R⁶, —R⁵—S(O)₂R⁶, —S(O)_{f} Het², —R⁵—S(O)₂Het², —S(O)₂NR⁶R⁷, —R⁵—S(O)₂NR⁶R⁷, —S(O)₂—R⁶—NR⁶R⁷,
      —NR⁶R⁷, —R⁵—NR⁶R⁷, —N(R⁶)Het², —N(R⁶)—R⁵cycloalkyl, —N(R⁶)—R⁵—Het²,
         —N(R⁶)—R⁶—OR⁷, —N(R⁶)—R⁵—S(O)_{f}R⁷, —N(R⁶)—R⁵—CN,
         —N(R⁶)—R⁵—NR⁶R⁷, —N(H)S(O)₂R⁶, —N(R⁶)—C(O)—NR⁶R⁷,
         —N(R⁶)—S(O)₂—NR⁶R⁷,
      —CN, —R⁵—CN and —NO₂; and
   when Ring B is a 9-10 membered aryl or 9-10 membered heteroaryl, then e is 0, 1 or 2 and
   each Z is the same or different and is independently selected from halo, alkyl, oxo, —OR⁶ and —NR⁶R⁷;
each Het² is the same or different and is independently heterocycle or heteroaryl, said heterocycle or heteroaryl having 1 or 2 heteroatoms selected from N, O and S and each optionally substituted with 1, 2 or 3 substituents which are the same or different and are each independently selected from:
   halo, C₁₋₃alkyl, haloC₁₋₃alkyl, C₁₋₃alkylene-O—C₁₋₃alkyl, OH, C₁₋₃alkylene-OH, oxo, C(O)(C₁₋₃alkyl), C(O)₂—C₁₋₃alkyl,
   C(O)—(C₁₋₃alkylene)-O(C₁₋₃alkyl), C(O)₂-benzyl, SO₃H, SO₂(C₁₋₃alkyl), C₁₋₃alkylene-SO₃H, C₁₋₃alkylene-SO₂(C₁₋₃alkyl), NH₂, N(H)C₁₋₃alkyl, N(C₁₋₃alkyl)₂,
   CN and C₁₋₃alkylene-CN;
Het³ is selected from 4-7 membered heterocycle and 5-7 membered heteroaryl, said heterocycle or heteroaryl having 1 or 2 heteroatoms selected from N, O and S and optionally substituted with 1 or 2 additional substituents which are the same or different and are each independently selected from halo, C₁₋₃alkyl, haloC₁₋₃alkyl, and O—C₁₋₃alkyl;
   each R⁶ and each R⁷ is the same or different and is independently H, alkyl or haloalkyl;
   or a pharmaceutically acceptable salt thereof.

In a second aspect, the present invention provides compounds of formula (I-i), (I-ii) or (I-iii)

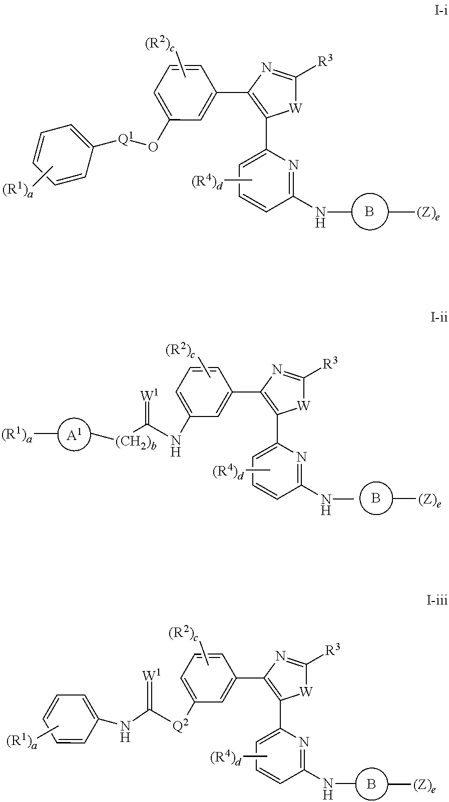

wherein all variables are as defined herein.

In a third aspect, the present invention provides compounds of formula (I-i-a) or (I-i-b):

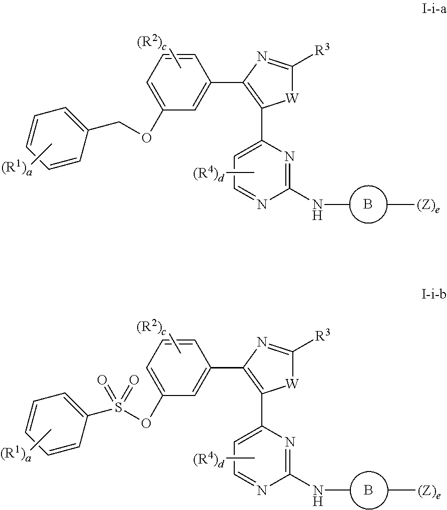

wherein all variables are as defined herein.

In a fourth aspect, the present invention provides compounds of formula (I-ii-a), (I-ii-b), (I-ii-c), or (I-ii-d):

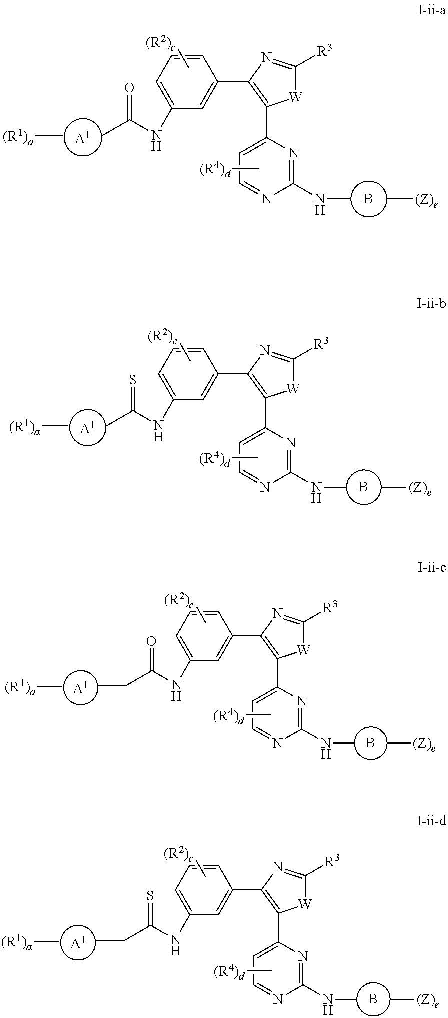

wherein all variables are as defined herein.

In a fifth aspect, the present invention provides compounds of formula (I-ii-1):

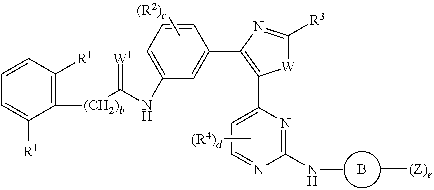

wherein all variables are as defined herein.

In a sixth aspect, the present invention provides compounds of formula (I-iii-a), (I-iii-b), (I-iii-c), and (I-iii-d):

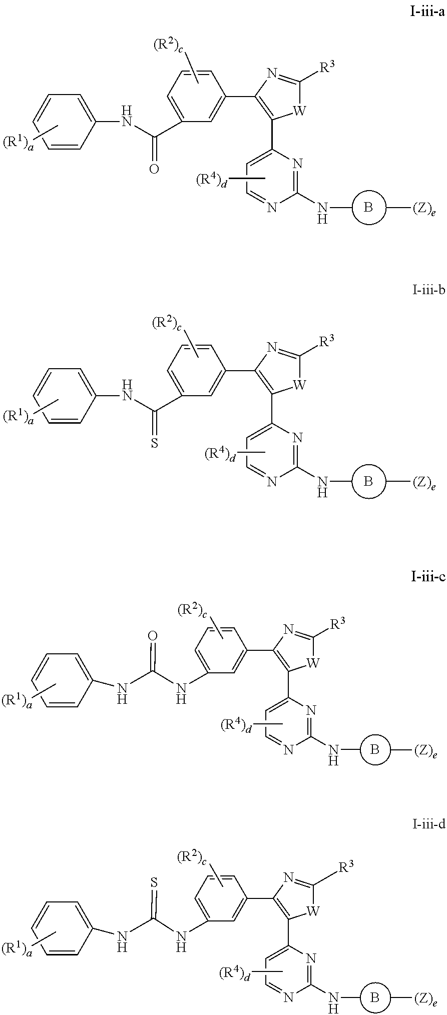

wherein all variables are as defined herein.

In a seventh aspect of the present invention, there is provided a compound selected from:
N-{3-[2-Amino-5-(2-{[3-{[2-(dimethylamino)ethyl]oxy}-4-(methyloxy)phenyl]amino}-4-pyrimidinyl)-1,3-thiazol-4-yl]phenyl}-2,6-difluoro-N-methylbenzamide;
N-(3-{2-Amino-5-[2-({3-chloro-4-[2-(dimethylamino)ethoxy]-phenyl}amino)pyrimidin-4-yl]-1,3-thiazol-4-yl}phenyl)-2,6-difluorobenzamide-formic acid
N-[3-(2-Amino-5-{2-[(3,4,5-trimethoxyphenyl)amino]pyrimidiN-4-yl}-1,3-thiazol-4-yl)phenyl]-2,6-difluorobenzamide;
N-[3-(2-(Dimethylamino)-5-{2-[(4-{[2-(dimethylamino)ethyl]oxy}phenyl)amino]-4-pyrimidinyl}-1,3-thiazol-4-yl)phenyl]-2,6-difluorobenzamide;
2,6-Difluoro-N-{3-[5-(2-{[4-(methyloxy)-3-(4-methyl-1-piperazinyl)phenyl]amino}-4-pyrimidinyl)-1,3-thiazol-4-yl]phenyl}benzamide;
N-[3-(5-{2-[(4-{[2-(Dimethylamino)ethyl]oxy}-3-fluorophenyl)amino]-4-pyrimidinyl}-2-ethyl-1,3-thiazol-4-yl)phenyl]-2,6-difluorobenzamide; and
N-[3-(5-{2-[(3-Chloro-4-{[2-(1-pyrrolidinyl)ethyl]oxy}phenyl)amino]-4-pyrimidinyl}-2-ethyl-1,3-thiazol-4-yl)phenyl]-2,6-difluorobenzamide;
   and pharmaceutically acceptable salts thereof.

In an eighth aspect, the present invention provides a pharmaceutical composition comprising a compound of formula (I) (or a sub-generic formula of formula (I) that is described herein) or a pharmaceutically acceptable salt thereof. In one embodiment, the pharmaceutical composition further comprises one or more of pharmaceutically acceptable carriers, diluents and excipients.

In a ninth aspect of the present invention, there is provided a method of treating a susceptible neoplasm in a mammal in need thereof, comprising administering to the mammal a therapeutically effective amount of a compound of formula (I) (or a sub-generic formula of formula (I) that is described herein) or a pharmaceutically acceptable salt thereof. Susceptible neoplasms include Barret's adenocarcinoma, billiary tract carcinomas, bladder cancer, breast cancer, cervical cancer, cholangiocarcinoma, central nervous system tumors including primary CNS tumors and secondary CNS tumors, colorectal cancer, esophageal cancer, gastric cancer, carcinoma of the head and neck, hematologic cancers including leukemias and lymphomas, hepatocellular carcinoma, lung cancer including small cell lung cancer, non-small cell lung cancer and squamous cell lung cancer, ovarian cancer, endometrial cancer, cervical cancer, pancreatic cancer, pituitary adenoma, prostate cancer, renal cancer, sarcoma, skin cancers including melanomas, thyroid cancers, and uterine cancer.

In another aspect of the present invention, there is provided a method of treating breast cancer, colorectal cancer, melanoma, non-small cell lung cancer, ovarian cancer, or thyroid cancer, in a mammal in need thereof, comprising administering to the mammal a therapeutically effective amount of a compound of formula (I) or a pharmaceutically acceptable salt thereof.

In another aspect of the present invention, there is provided a method of treating a susceptible neoplasm in a mammal in need thereof, comprising administering to the mammal a therapeutically effective amount of a compound selected from
N-{3-[2-Amino-5-(2-{[3-{[2-(dimethylamino)ethyl]oxy}-4-(methyloxy)phenyl]amino}-4-pyrimidinyl)-1,3-thiazol-4-yl]phenyl}-2,6-difluoro-N-methylbenzamide;
N-(3-{2-Amino-5-[2-({3-chloro-4-[2-(dimethylamino)ethoxy]-phenyl}amino)pyrimidin-4-yl]-1,3-thiazol-4-yl}phenyl)-2,6-difluorobenzamide-formic acid
N-[3-(2-Amino-5-{2-[(3,4,5-trimethoxyphenyl)amino]pyrimidiN-4-yl}-1,3-thiazol-4-yl)phenyl]-2,6-difluorobenzamide;
N-[3-(2-(Dimethylamino)-5-{2-[(4-{[2-(dimethylamino)ethyl]oxy}phenyl)amino]-4-pyrimidinyl}-1,3-thiazol-4-yl)phenyl]-2,6-difluorobenzamide;
2,6-Difluoro-N-{3-[5-(2-{[4-(methyloxy)-3-(4-methyl-1-piperazinyl)phenyl]amino}-4-pyrimidinyl)-1,3-thiazol-4-yl]phenyl}benzamide;
N-[3-(5-{2-[(4-{[2-(Dimethylamino)ethyl]oxy}-3-fluorophenyl)amino]-4-pyrimidinyl}-2-ethyl-1,3-thiazol-4-yl)phenyl]-2,6-difluorobenzamide; and
N-[3-(5-{2-[(3-Chloro-4-{[2-(1-pyrrolidinyl)ethyl]oxy}phenyl)amino]-4-pyrimidinyl}-2-ethyl-1,3-thiazol-4-yl)phenyl]-2,6-difluorobenzamide;
   and pharmaceutically acceptable salts thereof.

In another aspect of the present invention, there is provided a method of treating breast cancer, colorectal cancer, melanoma, non-small cell lung cancer, ovarian cancer, or thyroid cancer, in a mammal in need thereof, comprising administering to the mammal a therapeutically effective amount of a compound selected from
N-{3-[2-Amino-5-(2-{[3-{[2-(dimethylamino)ethyl]oxy}-4-(methyloxy)phenyl]amino}-4-pyrimidinyl)-1,3-thiazol-4-yl]phenyl}-2,6-difluoro-N-methylbenzamide;
N-(3-{2-Amino-5-[2-({3-chloro-4-[2-(dimethylamino)ethoxy]-phenyl}amino)pyrimidin-4-yl]-1,3-thiazol-4-yl}phenyl)-2,6-difluorobenzamide-formic acid
N-[3-(2-Amino-5-{2-[(3,4,5-trimethoxyphenyl)amino]pyrimidiN-4-yl}-1,3-thiazol-4-yl)phenyl]-2,6-difluorobenzamide;
N-[3-(2-(Dimethylamino)-5-{2-[(4-{[2-(dimethylamino)ethyl]oxy}phenyl)amino]-4-pyrimidinyl}-1,3-thiazol-4-yl)phenyl]-2,6-difluorobenzamide;
2,6-Difluoro-N-{3-[5-(2-{[4-(methyloxy)-3-(4-methyl-1-piperazinyl)phenyl]amino}-4-pyrimidinyl)-1,3-thiazol-4-yl]phenyl}benzamide;
N-[3-(5-{2-[(4-{[2-(Dimethylamino)ethyl]oxy}-3-fluorophenyl)amino]-4-pyrimidinyl}-2-ethyl-1,3-thiazol-4-yl)phenyl]-2,6-difluorobenzamide; and
N-[3-(5-{2-[(3-Chloro-4-{[2-(1-pyrrolidinyl)ethyl]oxy}phenyl)amino]-4-pyrimidinyl}-2-ethyl-1,3-thiazol-4-yl)phenyl]-2,6-difluorobenzamide;
   and pharmaceutically acceptable salts thereof.

In another aspect of the present invention, there is provided a process for preparing a compound of formula (I) (or a sub-generic formula of formula (I) that is described herein), wherein Y is moiety i, ii or iii, and all other variables are as defined herein. The process comprising reacting a compound of formula (VIII), (VIII-A), (VIII-B) or (XXVI):

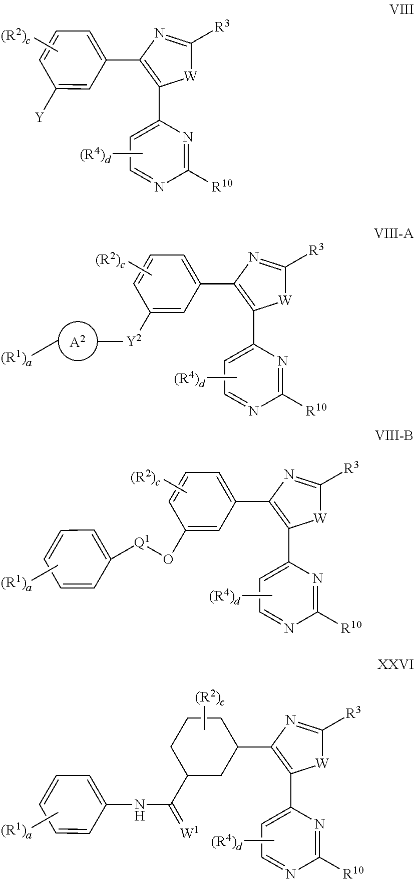

wherein R¹⁰ is halo or thiomethyl;
Y in formula (VIII) is moiety ii or moiety iii wherein Q² is —N(H)—;
Y² is —C(O)NH, —CH₂—C(O)NH—, or —N(H)C(O)N(H)—;
Ring A² is phenyl or Ring A¹;
and all other variables are as defined above;
   with an aniline of formula (IX):

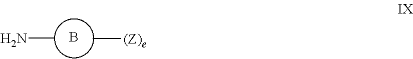

to prepare a compound of formula (I) wherein Y is moiety i, ii or iii, or a sub-generic formula of formula (I) that is described herein, particularly a compound of formula (I-i), (I-ii) or (I-iii).

In another aspect of the present invention, there is provided a process for preparing a compound of formula (I), wherein Y is moiety ii or moiety iii wherein Q² is —N(H)—, and all other variables are as defined herein. The process comprises reacting a compound of formula (XIV):

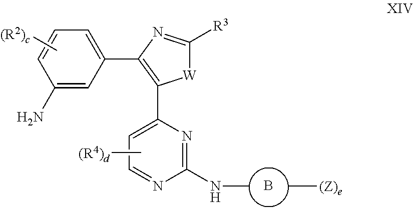

wherein all variables are as defined herein;
   with a compound of formula (VII-A):

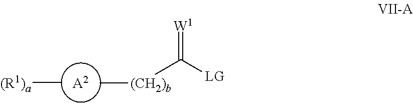

wherein Ring A² is phenyl or Ring A¹ and LG is a suitable leaving group; or a compound of formula (VII-B):

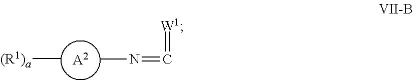

to prepare a compound of formula (I), particularly a compound of formula (I-ii) or (I-iii) wherein Q² is —N(H)—.

In another aspect of the present invention, there is provided a a process for preparing a compound of formula (I) (or a sub-generic formula of formula (I) that is described herein), wherein Y is moiety ii or moiety iii wherein Q² is —N(H)—, and all other variables are as defined herein. The process comprises reacting a compound of formula (XXXI):

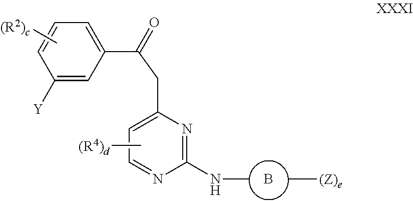

with a suitable brominating agent followed by reaction with one of:
i) a thiourea,
ii) a formamide,
iii) an amide,
iv) a thioamide, or
v) a urea;
   to prepare a compound of formula (I) (or a sub-generic formula of formula (I) that is described herein).

In another aspect of the present invention, there is provided a compound of formula (I), (including any particular sub-generic formula described herein) or a pharmaceutically acceptable salt thereof for use in therapy.

In another aspect, there is provided a compound of formula (I) (including any particular sub-generic formula described herein) or a pharmaceutically acceptable salt thereof for use in the treatment of a susceptible neoplasm (e.g., Barret's adenocarcinoma, billiary tract carcinomas, bladder cancer, breast cancer, cervical cancer, cholangiocarcinoma, central nervous system tumors including primary CNS tumors and secondary CNS tumors, colorectal cancer, esophageal cancer, gastric cancer, carcinoma of the head and neck, hematologic cancers including leukemias and lymphomas, hepatocellular carcinoma, lung cancer including small cell lung cancer, non-small cell lung cancer and squamous cell lung cancer, ovarian cancer, endometrial cancer, cervical cancer, pancreatic cancer, pituitary adenoma, prostate cancer, renal cancer, sarcoma, skin cancers including melanomas, thyroid cancers, and uterine cancer) in a mammal (e.g., human) in need thereof.

In another aspect, there is provided a compound of formula (I) (including any particular sub-generic formula described herein) or a pharmaceutically acceptable salt thereof for use in the treatment of breast cancer, colorectal cancer, melanoma, non-small cell lung cancer, ovarian cancer, or thyroid cancer in a mammal (e.g., human) in need thereof.

In another aspect, there is provided a compound selected from
N-{3-[2-Amino-5-(2-{[3-{[2-(dimethylamino)ethyl]oxy}-4-(methyloxy)phenyl]amino}-4-pyrimidinyl)-1,3-thiazol-4-yl]phenyl}-2,6-difluoro-N-methylbenzamide;
N-(3-{2-Amino-5-[2-({3-chloro-4-[2-(dimethylamino)ethoxy]-phenyl}amino)pyrimidin-4-yl]-1,3-thiazol-4-yl}phenyl)-2,6-difluorobenzamide-formic acid

N-[3-(2-Amino-5-{2-[(3,4,5-trimethoxyphenyl)amino]pyrimidiN-4-yl}-1,3-thiazol-4-yl)phenyl]-2,6-difluorobenzamide;
N-[3-(2-(Dimethylamino)-5-{2-[(4-{[2-(dimethylamino)ethyl]oxy}phenyl)amino]-4-pyrimidinyl}-1,3-thiazol-4-yl)phenyl]-2,6-difluorobenzamide;
2,6-Difluoro-N-{3-[5-(2-{[4-(methyloxy)-3-(4-methyl-1-piperazinyl)phenyl]amino}-4-pyrimidinyl)-1,3-thiazol-4-yl]phenyl}benzamide;
N-[3-(5-{2-[(4-{[2-(Dimethylamino)ethyl]oxy}-3-fluorophenyl)amino]-4-pyrimidinyl}-2-ethyl-1,3-thiazol-4-yl)phenyl]-2,6-difluorobenzamide; and
N-[3-(5-{2-[(3-Chloro-4-{[2-(1-pyrrolidinyl)ethyl]oxy}phenyl)amino]-4-pyrimidinyl}-2-ethyl-1,3-thiazol-4-yl)phenyl]-2,6-difluorobenzamide;
   and pharmaceutically acceptable salts thereof;
   for use in the treatment of a susceptible neoplasm (e.g., Barret's adenocarcinoma, billiary tract carcinomas, bladder cancer, breast cancer, cervical cancer, cholangiocarcinoma, central nervous system tumors including primary CNS tumors and secondary CNS tumors, colorectal cancer, esophageal cancer, gastric cancer, carcinoma of the head and neck, hematologic cancers including leukemias and lymphomas, hepatocellular carcinoma, lung cancer including small cell lung cancer, non-small cell lung cancer and squamous cell lung cancer, ovarian cancer, endometrial cancer, cervical cancer, pancreatic cancer, pituitary adenoma, prostate cancer, renal cancer, sarcoma, skin cancers including melanomas, thyroid cancers, and uterine cancer) in a mammal (e.g., human) in need thereof.

In another aspect, there is provided a compound selected from
N-{3-[2-Amino-5-(2-{[3-{[2-(dimethylamino)ethyl]oxy}-4-(methyloxy)phenyl]amino}-4-pyrimidinyl)-1,3-thiazol-4-yl]phenyl}-2,6-difluoro-N-methylbenzamide;
N-(3-{2-Amino-5-[2-({3-chloro-4-[2-(dimethylamino)ethoxy]-phenyl}amino)pyrimidin-4-yl]-1,3-thiazol-4-yl}phenyl)-2,6-difluorobenzamide-formic acid
N-[3-(2-Amino-5-{2-[(3,4,5-trimethoxyphenyl)amino]pyrimidiN-4-yl}-1,3-thiazol-4-yl)phenyl]-2,6-difluorobenzamide;
N-[3-(2-(Dimethylamino)-5-{2-[(4-{[2-(dimethylamino)ethyl]oxy}phenyl)amino]-4-pyrimidinyl}-1,3-thiazol-4-yl)phenyl]-2,6-difluorobenzamide;
2,6-Difluoro-N-{3-[5-(2-{[4-(methyloxy)-3-(4-methyl-1-piperazinyl)phenyl]amino}-4-pyrimidinyl)-1,3-thiazol-4-yl]phenyl}benzamide;
N-[3-(5-{2-[(4-{[2-(Dimethylamino)ethyl]oxy}-3-fluorophenyl)amino]-4-pyrimidinyl}-2-ethyl-1,3-thiazol-4-yl)phenyl]-2,6-difluorobenzamide; and
N-[3-(5-{2-[(3-Chloro-4-{[2-(1-pyrrolidinyl)ethyl]oxy}phenyl)amino]-4-pyrimidinyl}-2-ethyl-1,3-thiazol-4-yl)phenyl]-2,6-difluorobenzamide;
   and pharmaceutically acceptable salts thereof;
   for use in the treatment of breast cancer, colorectal cancer, melanoma, non-small cell lung cancer, ovarian cancer, or thyroid cancer in a mammal (e.g., human) in need thereof.

In a another aspect of the present invention, there is provided the use of a compound of formula (I) (including any particular sub-generic formula described herein) or a pharmaceutically acceptable salt thereof, in the preparation of a medicament for use in the treatment of a susceptible neoplasm (e.g., Barret's adenocarcinoma, billiary tract carcinomas, bladder cancer, breast cancer, cervical cancer, cholangiocarcinoma, central nervous system tumors including primary CNS tumors and secondary CNS tumors, colorectal cancer, esophageal cancer, gastric cancer, carcinoma of the head and neck, hematologic cancers including leukemias and lymphomas, hepatocellular carcinoma, lung cancer including small cell lung cancer, non-small cell lung cancer and squamous cell lung cancer, ovarian cancer, endometrial cancer, cervical cancer, pancreatic cancer, pituitary adenoma, prostate cancer, renal cancer, sarcoma, skin cancers including melanomas, thyroid cancers, and uterine cancer) in a mammal (e.g., human) in need thereof.

In a another aspect of the present invention, there is provided the use of a compound of formula (I) (including any particular sub-generic formula described herein) or a pharmaceutically acceptable salt thereof, in the preparation of a medicament for use in the treatment of breast cancer, colorectal cancer, melanoma, non-small cell lung cancer, ovarian cancer, or thyroid cancer in a mammal (e.g., human) in need thereof.

In a another aspect of the present invention, there is provided the use of a compound selected from
N-{3-[2-Amino-5-(2-{[3-{[2-(dimethylamino)ethyl]oxy}-4-(methyloxy)phenyl]amino}-4-pyrimidinyl)-1,3-thiazol-4-yl]phenyl}-2,6-difluoro-N-methylbenzamide;
N-(3-{2-Amino-5-[2-({3-chloro-4-[2-(dimethylamino)ethoxy]-phenyl}amino)pyrimidin-4-yl]-1,3-thiazol-4-yl}phenyl)-2,6-difluorobenzamide-formic acid
N-[3-(2-Amino-5-{2-[(3,4,5-trimethoxyphenyl)amino]pyrimidiN-4-yl}-1,3-thiazol-4-yl)phenyl]-2,6-difluorobenzamide;
N-[3-(2-(Dimethylamino)-5-{2-[(4-{[2-(dimethylamino)ethyl]oxy}phenyl)amino]-4-pyrimidinyl}-1,3-thiazol-4-yl)phenyl]-2,6-difluorobenzamide;
2,6-Difluoro-N-{3-[5-(2-{[4-(methyloxy)-3-(4-methyl-1-piperazinyl)phenyl]amino}-4-pyrimidinyl)-1,3-thiazol-4-yl]phenyl}benzamide;
N-[3-(5-{2-[(4-{[2-(Dimethylamino)ethyl]oxy}-3-fluorophenyl)amino]-4-pyrimidinyl}-2-ethyl-1,3-thiazol-4-yl)phenyl]-2,6-difluorobenzamide; and
N-[3-(5-{2-[(3-Chloro-4-{[2-(1-pyrrolidinyl)ethyl]oxy}phenyl)amino]-4-pyrimidinyl}-2-ethyl-1,3-thiazol-4-yl)phenyl]-2,6-difluorobenzamide;
   and pharmaceutically acceptable salts thereof;
   for the preparation of a medicament for the treatment of a susceptible neoplasm (e.g., Barret's adenocarcinoma, billiary tract carcinomas, bladder cancer, breast cancer, cervical cancer, cholangiocarcinoma, central nervous system tumors including primary CNS tumors and secondary CNS tumors, colorectal cancer, esophageal cancer, gastric cancer, carcinoma of the head and neck, hematologic cancers including leukemias and lymphomas, hepatocellular carcinoma, lung cancer including small cell lung cancer, non-small cell lung cancer and squamous cell lung cancer, ovarian cancer, endometrial cancer, cervical cancer, pancreatic cancer, pituitary adenoma, prostate cancer, renal cancer, sarcoma, skin cancers including melanomas, thyroid cancers, and uterine cancer) in a mammal (e.g., human) in need thereof.

In a another aspect of the present invention, there is provided the use of a compound selected from
N-{3-[2-Amino-5-(2-{[3-{[2-(dimethylamino)ethyl]oxy}-4-(methyloxy)phenyl]amino}-4-pyrimidinyl)-1,3-thiazol-4-yl]phenyl}-2,6-difluoro-N-methylbenzamide;
N-(3-{2-Amino-5-[2-({3-chloro-4-[2-(dimethylamino)ethoxy]-phenyl}amino)pyrimidin-4-yl]-1,3-thiazol-4-yl}phenyl)-2,6-difluorobenzamide-formic acid
N-[3-(2-Amino-5-{2-[(3,4,5-trimethoxyphenyl)amino]pyrimidiN-4-yl}-1,3-thiazol-4-yl)phenyl]-2,6-difluorobenzamide;
N-[3-(2-(Dimethylamino)-5-{2-[(4-{[2-(dimethylamino)ethyl]oxy}phenyl)amino]-4-pyrimidinyl}-1,3-thiazol-4-yl)phenyl]-2,6-difluorobenzamide;
2,6-Difluoro-N-{3-[5-(2-{[4-(methyloxy)-3-(4-methyl-1-piperazinyl)phenyl]amino}-4-pyrimidinyl)-1,3-thiazol-4-yl]phenyl}benzamide;
N-[3-(5-{2-[(4-{[2-(Dimethylamino)ethyl]oxy}-3-fluorophenyl)amino]-4-pyrimidinyl}-2-ethyl-1,3-thiazol-4-yl)phenyl]-2,6-difluorobenzamide; and
N-[3-(5-{2-[(3-Chloro-4-{[2-(1-pyrrolidinyl)ethyl]oxy}phenyl)amino]-4-pyrimidinyl}-2-ethyl-1,3-thiazol-4-yl)phenyl]-2,6-difluorobenzamide;
   and pharmaceutically acceptable salts thereof;
   for the preparation of a medicament for the treatment of breast cancer, colorectal cancer, melanoma, non-small cell lung cancer, ovarian cancer, or thyroid cancer in a mammal (e.g., human) in need thereof.

In another aspect of the present invention, there is provided a pharmaceutical composition comprising a compound of formula (I) (including any particular sub-generic formula described herein) or a pharmaceutically acceptable salt thereof for use in the treatment of a susceptible neoplasm (e.g., Barret's adenocarcinoma, billiary tract carcinomas, bladder cancer, breast cancer, cervical cancer, cholangiocarcinoma, central nervous system tumors including primary CNS tumors and secondary CNS tumors, colorectal cancer, esophageal cancer, gastric cancer, carcinoma of the head and neck, hematologic cancers including leukemias and lymphomas, hepatocellular carcinoma, lung cancer including small cell lung cancer, non-small cell lung cancer and squamous cell lung cancer, ovarian cancer, endometrial cancer, cervical cancer, pancreatic cancer, pituitary adenoma, prostate cancer, renal cancer, sarcoma, skin cancers including melanomas, thyroid cancers, and uterine cancer) in a mammal (e.g., human) in need thereof.

In another aspect of the present invention, there is provided a pharmaceutical composition comprising a compound of formula (I) (including any particular sub-generic formula described herein) or a pharmaceutically acceptable salt thereof for use in the treatment of breast cancer, colorectal cancer, melanoma, non-small cell lung cancer, ovarian cancer, or thyroid cancer in a mammal (e.g., human) in need thereof.

In another aspect of the present invention, there is provided a pharmaceutical composition comprising a compound selected from
N-{3-[2-Amino-5-(2-{[3-{[2-(dimethylamino)ethyl]oxy}-4-(methyloxy)phenyl]amino}-4-pyrimidinyl)-1,3-thiazol-4-yl]phenyl}-2,6-difluoro-N-methylbenzamide;
N-(3-{2-Amino-5-[2-({3-chloro-4-[2-(dimethylamino)ethoxy]-phenyl}amino)pyrimidin-4-yl]-1,3-thiazol-4-yl}phenyl)-2,6-difluorobenzamide-formic acid
N-[3-(2-Amino-5-{2-[(3,4,5-trimethoxyphenyl)amino]pyrimidiN-4-yl}-1,3-thiazol-4-yl)phenyl]-2,6-difluorobenzamide;
N-[3-(2-(Dimethylamino)-5-{2-[(4-{[2-(dimethylamino)ethyl]oxy}phenyl)amino]-4-pyrimidinyl}-1,3-thiazol-4-yl)phenyl]-2,6-difluorobenzamide;
2,6-Difluoro-N-{3-[5-(2-{[4-(methyloxy)-3-(4-methyl-1-piperazinyl)phenyl]amino}-4-pyrimidinyl)-1,3-thiazol-4-yl]phenyl}benzamide;
N-[3-(5-{2-[(4-{[2-(Dimethylamino)ethyl]oxy}-3-fluorophenyl)amino]-4-pyrimidinyl}-2-ethyl-1,3-thiazol-4-yl)phenyl]-2,6-difluorobenzamide; and
N-[3-(5-{2-[(3-Chloro-4-{[2-(1-pyrrolidinyl)ethyl]oxy}phenyl)amino]-4-pyrimidinyl}-2-ethyl-1,3-thiazol-4-yl)phenyl]-2,6-difluorobenzamide;
   and pharmaceutically acceptable salts thereof;
   for use in the treatment of a susceptible neoplasm (e.g., Barret's adenocarcinoma, billiary tract carcinomas, bladder cancer, breast cancer, cervical cancer, cholangiocarcinoma, central nervous system tumors including primary CNS tumors and secondary CNS tumors, colorectal cancer, esophageal cancer, gastric cancer, carcinoma of the head and neck, hematologic cancers including leukemias and lymphomas, hepatocellular carcinoma, lung cancer including small cell lung cancer, non-small cell lung cancer and squamous cell lung cancer, ovarian cancer, endometrial cancer, cervical cancer, pancreatic cancer, pituitary adenoma, prostate cancer, renal cancer, sarcoma, skin cancers including melanomas, thyroid cancers, and uterine cancer) in a mammal (e.g., human) in need thereof.

In another aspect of the present invention, there is provided a pharmaceutical composition comprising a compound selected from
N-{3-[2-Amino-5-(2-{[3-{[2-(dimethylamino)ethyl]oxy}-4-(methyloxy)phenyl]amino}-4-pyrimidinyl)-1,3-thiazol-4-yl]phenyl}-2,6-difluoro-N-methylbenzamide;
N-(3-{2-Amino-5-[2-({3-chloro-4-[2-(dimethylamino)ethoxy]-phenyl}amino)pyrimidin-4-yl]-1,3-thiazol-4-yl}phenyl)-2,6-difluorobenzamide-formic acid
N-[3-(2-Amino-5-{2-[(3,4,5-trimethoxyphenyl)amino]pyrimidiN-4-yl}-1,3-thiazol-4-yl)phenyl]-2,6-difluorobenzamide;
N-[3-(2-(Dimethylamino)-5-{2-[(4-{[2-(dimethylamino)ethyl]oxy}phenyl)amino]-4-pyrimidinyl}-1,3-thiazol-4-yl)phenyl]-2,6-difluorobenzamide;
2,6-Difluoro-N-{3-[5-(2-{[4-(methyloxy)-3-(4-methyl-1-piperazinyl)phenyl]amino}-4-pyrimidinyl)-1,3-thiazol-4-yl]phenyl}benzamide;
N-[3-(5-{2-[(4-{[2-(Dimethylamino)ethyl]oxy}-3-fluorophenyl)amino]-4-pyrimidinyl}-2-ethyl-1,3-thiazol-4-yl)phenyl]-2,6-difluorobenzamide; and
N-[3-(5-{2-[(3-Chloro-4-{[2-(1-pyrrolidinyl)ethyl]oxy}phenyl)amino]-4-pyrimidinyl}-2-ethyl-1,3-thiazol-4-yl)phenyl]-2,6-difluorobenzamide;
   and pharmaceutically acceptable salts thereof;
   for use in the treatment of breast cancer, colorectal cancer, melanoma, non-small cell lung cancer, ovarian cancer, or thyroid cancer in a mammal (e.g., human) in need thereof.

These and other aspects of the invention are described further in the Detailed Description and Examples which follow.

### DETAILED DESCRIPTION OF THE INVENTION

As used herein the term “ErbB family kinase” refers to ErbB kinases including EGFR (also known as ErbB1), ErbB2, and ErbB4.

As used herein, the term “Raf family kinase” refers to Raf kinases including A-Raf, B-Raf and c-Raf (also known as Raf-1).

As used herein, “compound(s) of formula (I)” means any compound having the structural formula (I) as defined by the variable definitions provided, amorphous and crystal forms, including one or more polymorphic forms and mixtures thereof. In the case of compounds of formula (I) which possess one or more chiral centers, the compounds may be in the form of a racemic mixture, or one or more isomerically enriched or pure stereoisomers, including enantiomers and diastereomers thereof. In such embodiments, “compound(s) of formula (I)” includes the racemic form as well as the enriched or pure enantiomers and diastereomers. Enantiomerically enriched or pure compounds will be designated using conventional nomenclature, including the designations +, −, R, S, d, l, D and L, according to the predominant isomer present. Where a compound of the invention contains an alkenyl or alkenylene group, cis (E) and trans (Z) isomerism may also occur. In such embodiments, “compound(s) of formula (I)” includes the individual stereoisomers of the compound of the invention, which will be indicated using conventional, cis/trans nomenclature. It should also be understood that compounds of formula (I) may exist in tautomeric forms other than that shown in the formula and alternative tautomeric forms are also included within “compound(s) of formula (I).”

As used herein, “compound(s) of the invention” means a compound of formula (I) (as defined above, and including any of the sub-generic formulas of formula (I) described herein, the compound being in any version, i.e., as the free base or as a pharmaceutically acceptable salt thereof. The compound as any version may be in any form, including amorphous or crystalline forms, specific polymorphic forms, solvates, including hydrates (e.g., mono-, di- and hemi-hydrates), and mixtures of various forms.

Intermediates may also be present as salts. In reference to intermediates, the phrase “compound(s) of formula (number)” means a compound having that structural formula or a pharmaceutically acceptable salt thereof.

The term “alkyl” as used herein refers to linear or branched hydrocarbon chains having from 1 to 8 carbon atoms, unless a different number of atoms is specified. Examples of “alkyl” as used herein include, but are not limited to, methyl, ethyl, n-propyl, n-butyl, n-pentyl, isobutyl, isopropyl, and tert-butyl. Similarly, the term “alkylene” refers to linear or branched divalent hydrocarbon chains containing from 1 to 8 carbon atoms, unless a different number of atoms is specified. Examples of “alkylene” as used herein include, but are not limited to, methylene, ethylene, propylene, butylene, and isobutylene.

As used herein, the term “alkenyl” refers to linear or branched hydrocarbon chains having from 2 to 8 carbon atoms, unless a different number of atoms is specified, and at least one and up to three carbon-carbon double bonds. Examples of “alkenyl” as used herein include, but are not limited to ethenyl and propenyl.

As used herein, the term “cycloalkyl” refers to a saturated monocyclic carbocyclic ring or a saturated bicyclic carbocyclic ring, including fused and spiro systems, having from 3 to 8 carbon atoms, unless a different number of atoms is specified. In one embodiment, “cycloalkyl” refers to a saturated monocyclic carbocyclic ring having from 3 to 8 carbon atoms, unless a different number is specified. “Cycloalkyl” includes by way of example cyclopropyl, cyclobutyl, cyclopentyl, cyclohexyl, cycloheptyl and cyclooctyl. Preferred cycloalkyl groups include substituted and unsubstituted C₃₋₆cycloalkyl.

The terms “halo” and “halogen” are synonymous and refer to fluoro, chloro, bromo and iodo.

As used herein, “haloalkyl” refers to an alkyl, as defined above, substituted by one or more halogen atoms, fluoro, chloro, bromo or iodo. Where the haloalkyl group has less than 8 carbon atoms, the number of carbon atoms in the group is indicated as, for example, “haloC₁₋₃alkyl”, which indicates that the haloalkyl group has 1, 2 or 3 carbon atoms. Examples of haloalkyl as used herein include, but are not limited to fluoromethyl, difluoromethyl, trifluoromethyl, fluoroethyl, trifluoroethyl and the like. The term “oxo” as used herein refers to the group ═O attached directly to a carbon atom of a hydrocarbon ring (e.g., cycloalkyl or cycloalkenyl) or a C, N or S of a heterocyclic or heteroaryl ring to result in oxides, N-oxides, sulfones and sulfoxides.

As used herein, the term “aryl” refers to aromatic monocyclic carbocyclic groups, aromatic fused bicyclic carbocyclic groups, and bicyclic fused carbocyclic groups which have both aromatic and non-aromatic rings, each having the specified number of carbon atoms. In all embodiments wherein the compound of formula (I) includes two or more aryl groups, the aryl groups may be the same or different and are independently selected. Examples of particular aryl groups include but are not limited to phenyl, indenyl and naphthyl.

As used herein, the terms “heterocycle” and “heterocyclic” are synonymous and refer to monocyclic saturated or unsaturated non-aromatic groups, and fused bicyclic saturated or unsaturated non-aromatic groups, each having from 4 to 10 members (unless a different number of members is specified), and spiro systems having from 7 to 12 members (unless a different number of members is specified). The monocyclic, bicyclic and spiro systems, include 1, 2 or 3 heteroatoms selected from N, O and S, unless a different number of heteroatoms is specified. In one embodiment, “heterocycle” and “heterocyclic” refer to monocyclic saturated or unsaturated non-aromatic groups and fused bicyclic saturated or unsaturated non-aromatic groups, each having from 4 to 10 members (unless a different number of members is specified) including 1, 2 or 3 heteroatoms selected from N, O and S, unless a different number of heteroatoms is specified. In embodiments wherein the heterocycle has 6 or fewer members, it should be clear that such embodiments do not include 7-12 membered spiro systems. In all embodiments wherein the heterocycle includes 2 or more heteroatoms, the heteroatoms may be the same or different and are independently selected from N, O and S. In all embodiments wherein the compound of formula (I) includes two or more heterocyclic, groups, the heterocyclic groups may be the same or different and are independently selected. Examples of particular heterocyclic groups include but are not limited to tetrahydrofuran, dihydropyran, tetrahydropyran, pyran, thietane, 1,4-dioxane, 1,3-dioxane, 1,3-dioxalane, piperidine, piperazine, pyrrolidine, morpholine, thiomorpholine, thiazolidine, oxazolidine, tetrahydrothiopyran, tetrahydrothiophene and the like.

As used herein, the term “N-heterocycle” refers to monocyclic saturated or unsaturated non-aromatic groups, fused bicyclic saturated or unsaturated non-aromatic groups and each having from 4 to 10 members (unless a different number of members is specified), and spiro systems having from 7 to 12 members (unless a different number of members is specified). The monocyclic, bicyclic and spiro systems, include at least one N and optionally 1 or 2 additional heteroatoms selected from N, O and S, unless a different number of additional heteroatoms is specified. In one embodiment, “N-heterocycle” refers to monocyclic saturated or unsaturated non-aromatic groups and fused bicyclic saturated or unsaturated non-aromatic groups, each having from 4 to 10 members (unless a different number of members is specified) including at least one N and optionally 1 or 2 additional heteroatoms selected from N, O and S, unless a different number of additional heteroatoms is specified. In embodiments wherein the N-heterocycle has 6 or fewer members, it should be clear that such embodiments do not include 7-12 membered spiro systems. By “additional heteroatoms” is meant 1 or 2 heteroatoms in addition to the N already specified in the N-heterocycle ring. In all embodiments wherein the heterocycle includes 1 or more additional heteroatoms, the heteroatoms may be the same or different and are independently selected from N, O and S. In all embodiments wherein the compound of formula (I) includes two or more N-heterocyclic groups, the N-heterocyclic groups may be the same or different and are independently selected. Examples of N-heterocycles include piperidine, piperazine, pyrrolidine, morpholine, thiomorpholine and the like.

As used herein, the term “heteroaryl” refers to aromatic, monocyclic groups, aromatic fused bicyclic groups and fused bicyclic groups which have both aromatic and non-aromatic rings, each having from 5 to 10 members (unless a different number of members is specified) including 1, 2, 3, or 4 heteroatoms (particularly 1, 2 or 3) selected from N, O and S, unless a different number of heteroatoms is specified. In all embodiments wherein the heteroaryl includes 2 or more heteroatoms, the heteroatoms may be the same or different and are independently selected from N, O and S. In all embodiments wherein the compound of formula (I) includes two or more heteroaryl groups, the heteroaryl groups may be the same or different and are independently selected. Examples of particular heteroaryl groups include but are not limited to furan, thiophene, pyrrole, imidazole, pyrazole, triazole, tetrazole, thiazole, oxazole, isoxazole, oxadiazole, thiadiazole, isothiazole, pyridine, pyridazine, pyrazine, pyrimidine, triazine, quinoline, tetrahydroquinoline, isoquinoline, tetrahydroisoquinoline, benzofuran, benzothiophene, indole, indoline, indazole, benzodioxane, benzodioxin, benzodithiane, benzoxazine, benzopiperidine and benzopiperazine.

As used herein, the term “N-heteroaryl” refers to aromatic, monocyclic groups, aromatic fused bicyclic groups and fused bicyclic groups which have both aromatic and non-aromatic rings, each having from 5 to 10 members (unless a different number of members is specified) including at least one N and optionally 1, 2 or 3 (particularly 1 or 2) additional heteroatoms selected from N, O and S, unless a different number of heteroatoms is specified. By “additional heteroatoms” is meant 1, 2 or 3 heteroatoms in addition to the N already specified in the N-heteroaryl ring. In all embodiments wherein the heteroaryl includes 1 or more additional heteroatoms, the heteroatoms may be the same or different and are independently selected from N, O and S. In all embodiments wherein the compound of formula (I) includes two or more N-heteroaryl groups, the N-heteroaryl groups may be the same or different and are independently selected. Examples of N-heteroaryls include pyrrole, imidazole, pyrazole, thiazole, isoxazole, pyridine, pyridazine, pyrazine, pyrimidine, triazine, quinoline, isoquinoline, indole, indoline, benzopiperidine and benzopiperzine.

As used herein, the term “members” (and variants thereof e.g., “membered”) in the context of heterocyclic and heteroaryl groups refers to the total number of ring atoms, including carbon and heteroatoms N, O and/or S. Thus, an example of a 6-membered heterocyclic ring is piperidine and an example of a 6-membered heteroaryl ring is pyridine.

As used herein, the term “optionally substituted” means unsubstituted groups or rings (e.g., cycloalkyl, heterocycle, and heteroaryl rings) and those rings substituted with one or more specified substituents.

The present invention provides compounds of formula (I):

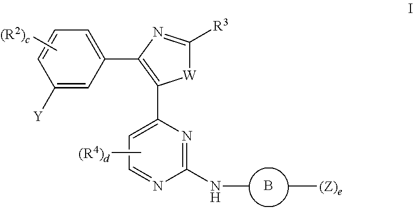

wherein:
Y is a moiety selected from i, ii, and

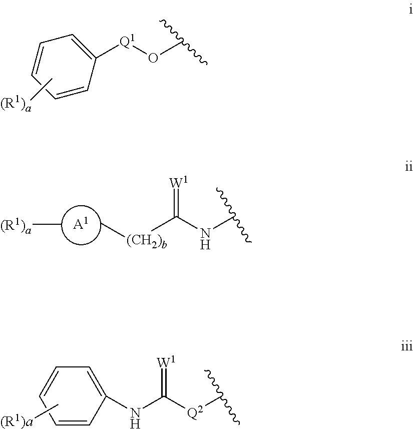

wherein:
a is 0, 1, 2 or 3;
each R¹ is the same or different and is independently selected from halo, alkyl, haloalkyl, —OR⁶, —R⁵—OR⁶, —C(O)R⁶, —CO₂R⁶, —S(O)_{f}R⁶, —R⁵—S(O)_{f}R⁶, —NR⁶R⁷, —R⁵—NR⁶R⁷, —CN and —R⁵—CN;
f is 0, 1 or 2;
Q¹ is —CH₂— or —SO₂—;
Ring A¹ is cycloalkyl, phenyl or 5-10 membered heteroaryl having 1 or 2 heteroatoms selected from N, O and S;
b is 0 or 1;
W¹ is O or S;
Q² is a bond or —N(H)—;
   c is 0, 1 or 2;
each R² is the same or different and is independently selected from halo, alkyl, haloalkyl, —OR⁶, —S(O)_{f}R⁶, —NR⁶R⁷, —CN and —NO₂;

### W is —O— or —S—;

R³ is selected from H, alkyl, haloalkyl, alkenyl, cycloalkyl, —R⁵-cycloalkyl, Ph, Het, —R⁵—OR⁶, —R⁵—S(O)_{f}R⁶, —R⁵—S(O)₂—NR⁶R⁷, —NR⁶R⁷, —N(R⁶)-cycloalkyl, —N(R⁶)Ph, —N(R⁶)Het, —N(R⁶)R⁵—Het, —N(R⁶)—R⁵—OR⁷, —N(R⁶)—R⁵—NR⁶R⁷, —N(H)C(O)R⁶, —R⁶—N(H)C(O)R⁶, —N(R⁶)—C(O)—NR⁶R⁷, —N(H)SO₂R⁶, —N(R⁶)—R⁵—S(O)_{f}R⁷, and —N(R⁶)—S(O)₂—NR⁶R⁷,
   wherein each of said cycloalkyl is optionally substituted with 1 or 2 substituents which are the same or different and are each independently selected from halo, haloalkyl, OH, O—C₁₋₃alkyl, oxo, S(C₁₋₃alkyl), SO₂, NH₂, N(H)C₁₋₃alkyl, and N(C₁₋₃alkyl)₂;
      d is 0, 1 or 2;
each R⁴ is the same or different and is independently selected from halo, alkyl, haloalkyl, —OR⁶, —S(O)_{f}R⁶, —NR⁶R⁷, —CN and —NO₂;
each Ph is the same or different and is independently phenyl optionally substituted with 1, 2 or 3 substituents which are the same or different and are each independently selected from halo, C₁₋₃alkyl, haloC₁₋₃alkyl, O—C₁₋₃alkyl, C₁₋₃alkylene-O—C₁₋₃alkyl, OH, C₁₋₃alkylene-OH, NH₂, N(H)C₁₋₃alkyl, N(C₁₋₃alkyl)₂, CN and NO₂;
each Het is the same or different and is independently selected from 4-6 membered heterocycle having 1 or 2 heteroatoms selected from N, O and S and optionally substituted with 1, 2 or 3 substituents which are the same or different and are each independently selected from halo, C₁₋₃alkyl, haloC₁₋₃alkyl, O—C₁₋₃alkyl, C₁₋₃alkylene-O—C₁₋₃alkyl, OH, C₁₋₃alkylene-OH, oxo, C(O)(C₁₋₃alkyl), C(O)NH₂, C(O)N(C₁₋₃alkyl)₂, SO₃(H), SO₂(C₁₋₃alkyl), C₁₋₃alkylene-SO₃(H), C₁₋₃alkylene-SO₂(C₁₋₃alkyl), NH₂, N(H)C₁₋₃alkyl, N(C₁₋₃alkyl)₂, CN, —CH₂CN, and NO₂;
   each R⁵ is the same or different and is independently C₁₋₄alkylene;
Ring B is selected from phenyl, 9-10 membered aryl, 5-6 membered heteroaryl, and 9-10 membered heteroaryl, each heteroaryl having 1, 2 or 3 heteroatoms selected from N, O and S;
   wherein when Ring B is selected from phenyl and 5-6 membered heteroaryl, then e is 0, 1, 2 or 3; and
   each Z is the same or different and is independently selected from:
      halo, alkyl, haloalkyl, alkenyl,
      Het², —R⁵Het², Het³-Het²,
      oxo, —OR⁶, —R⁵—OR⁶, —O—R⁵—OR⁶, —OHet², —O—R⁵—Het², —O—R⁵—NR⁶R⁷,
      —O—R⁵—S(O)₂R⁶, —C(O)NR⁶R⁷, —R⁵—C(O)NR⁶R⁷, —CO₂R⁶, —R⁵—CO₂R⁶,
      —S(O)_{f}R⁶, —R⁵—S(O)₂R⁶, —S(O)_{f} Het², —R⁵—S(O)₂Het², —S(O)₂NR⁶R⁷, —R⁵—S(O)₂NR⁶R⁷, —S(O)₂—R⁵—NR⁶R⁷,
      —NR⁶R⁷, —R⁵—NR⁶R⁷, —N(R⁶)Het², —N(R⁶)—R⁵cycloalkyl, —N(R⁶)—R⁵—Het²,
         —N(R⁶)—R⁵—OR⁷, —N(R⁶)—R⁵—S(O)_{f}R⁷, —N(R⁶)—R⁵—CN,
         —N(R⁶)—R⁵—NR⁶R⁷, —N(H)S(O)₂R⁶, —N(R⁶)—C(O)—NR⁶R⁷,
         —N(R⁶)—S(O)₂—NR⁶R⁷,
      CN, —R⁵—CN and —NO₂; and
   when Ring B is a 9-10 membered aryl or 9-10 membered heteroaryl, then e is 0, 1 or 2 and
   each Z is the same or different and is independently selected from halo, alkyl, oxo, —OR⁶ and —NR⁶R⁷;
each Het² is the same or different and is independently heterocycle or heteroaryl, said heterocycle or heteroaryl having 1 or 2 heteroatoms selected from N, O and S and each optionally substituted with 1, 2 or 3 substituents which are the same or different and are each independently selected from:
   halo, C₁₋₃alkyl, haloC₁₋₃alkyl, O—C₁₋₃alkyl, C₁₋₃alkylene-O—C₁₋₃alkyl, OH, C₁₋₃alkylene-OH, oxo, C(O)(C₁₋₃alkyl), C(O)₂—C₁₋₃alkyl,
   C(O)—(C₁₋₃alkylene)-O(C₁₋₃alkyl), C(O)₂-benzyl, SO₃H, SO₂(C₁₋₃alkyl), C₁₋₃alkylene-SO₃H, C₁₋₃alkylene-SO₂(C₁₋₃alkyl), NH₂, N(H)C₁₋₃alkyl, N(C₁₋₃alkyl)₂,
   CN and C₁₋₃alkylene-CN;
Het³ is selected from 4-7 membered heterocycle and 5-7 membered heteroaryl, said heterocycle or heteroaryl having 1 or 2 heteroatoms selected from N, O and S and optionally substituted with 1 or 2 additional substituents which are the same or different and are each independently selected from halo, C₁₋₃alkyl, haloC₁₋₃alkyl, and O—C₁₋₃alkyl;
   each R⁶ and each R⁷ is the same or different and is independently H, alkyl or haloalkyl;
   and pharmaceutically acceptable salts thereof.

The compounds of the invention are described in the conventional manner employing variables to represent a number of possible substituents or groups. The original, particular and preferred definitions of variables described herein apply equally to compounds of formula (I), compounds of the invention and sub-generic formulas of formula (I) that are described herein. For brevity, the following description will generally refer to “compounds of the invention” rather than to all, as compounds of the invention encompasses all compounds of formula (I) including sub-generic formulas. It should be understood that the definition of variables utilized to describe the compounds of the invention will be selected in light of the knowledge possessed by the ordinarily skilled organic chemist such that embodiments which such chemist would consider to be obviously inoperative or unstable are avoided. For example, the organic chemist of ordinary skill in the art would appreciate that moieties such as —N(H)CH₂F, —N(H)CH₂NH₂, —OCH₂NH₂, and the like, result in potentially, unstable acetals, aminals or iminium ions. As such, the present invention should be understood such that the variables are defined in a manner which avoids such embodiments.

For ease of reference, portions of formula (I) may sometimes be referred to herein as a “head” portion or a “tail” portion. The phenyl substituted by moiety Y defines the “head” portion and the “tail” portion is indicated by the box in the following illustration:

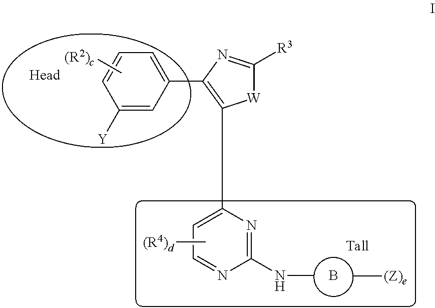

Considering the definitions of moiety Y, the following formulas illustrate compounds within the scope of the invention.

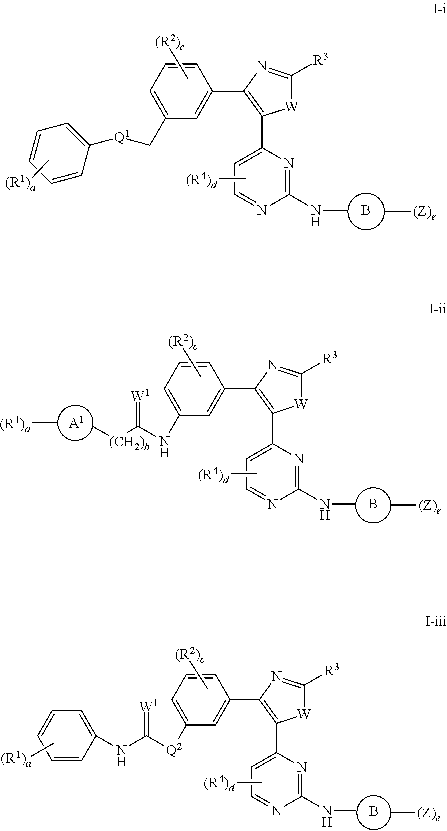

wherein all variables are as defined herein.

Two specific embodiments of compounds of formula (I-i) above may be illustrated as formulas (I-i-a) and (I-i-b):

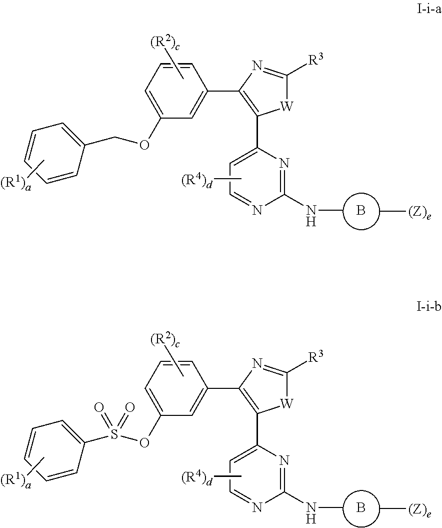

wherein all variables are as defined herein.

Considering first, the compounds of the invention defined wherein Y is a moiety (i), particular embodiments are defined wherein a of (R¹)ₐ is 0, 1 or 2.

In those embodiments of the compounds of the invention (irrespective of the definition of Y) wherein a is 1, 2 or 3, each R¹ may be bound to the phenyl or Ring A¹ through any suitable carbon or heteroatom (to provide, for example, N-methyl or N-oxides). In certain embodiments, wherein a is any of 1, 2 or 3, each R¹ is the same or different and is independently selected from halo, alkyl, haloalkyl, —OR⁶, —NR⁶R⁷, —CN and R⁵—CN, or any subset thereof. In one particular embodiment, each R^{I} is the same or different and is independently selected from F, Cl, alkyl haloalkyl and —OR⁶ or any subset thereof. Specific examples of groups defining R¹ include but are not limited to F, Cl, Br, CH₃, CF₃, CH₂CH₃, CH(CH₃)₂, OCH₃, OCF₃, OCH₂CH₃, NH₂, N(H)alkyl (e.g., N(H)CH₃), and CN. In one example of a preferred embodiment, each R¹ is the same or different and is independently selected from F, Cl, CH₃, CF₃, OCH₃, and OCF₃ or any subset thereof. In one preferred embodiment, each R¹ is F.

In one particular embodiment of compounds of the invention wherein Y is moiety i, a is 0 and thus the terminal phenyl of the head portion is unsubstituted.

Four specific embodiments of compounds of formula (I-ii) above may be illustrated as formulas (I-ii-a), (I-ii-b), (I-ii-c), and (I-ii-d):

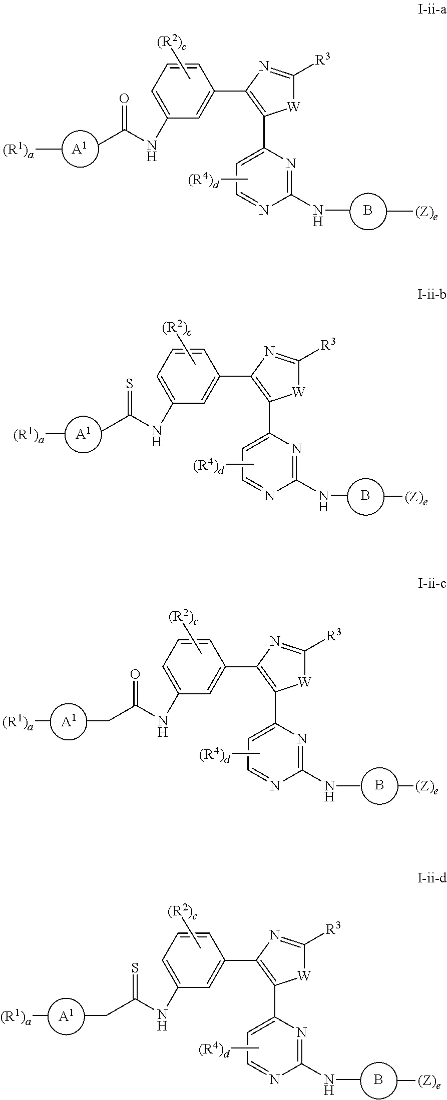

wherein all variables are as defined herein.

Compounds of the invention defined by formulas (I-ii-a) and (I-ii-c), i.e., wherein W¹ of moiety ii is 0, are generally preferred over compounds of formulas (I-ii-b) and (I-ii-d). For compounds of the invention wherein Y is moiety ii (i.e., compounds defined by formula (I-ii) including formulas (I-ii-a), (I-ii-b), (I-ii-c), and (I-ii-d)), particular embodiments are defined wherein a of (R¹)ₐ is 0, 1 or 2. Particular embodiments are defined wherein a is 0 or 2. In one specific embodiment, a is 0. In another specific embodiment, a is 1. In a preferred embodiment, a is 2. In one preferred embodiment, the compounds of the invention are defined by formula (I-ii-a), and a is 2.

In certain embodiments, wherein Y is moiety ii and a is any of 1, 2 or 3, each R¹ is the same or different and is independently selected from halo, alkyl, haloalkyl, —OR⁶, —NR⁶R⁷, —CN and R⁵—CN, or any subset thereof. In those embodiments wherein Y is moiety ii and R¹ is —OR⁶, where R⁶ is H, it will be understood that when Ring A¹ is a heteroaryl, the compounds of the invention include the tautomeric form wherein the heteroaryl Ring A¹ is substituted by oxo. In one particular embodiment, each R¹ is the same or different and is independently selected from F, Cl, alkyl haloalkyl and —OR⁶ or any subset thereof. Specific examples of groups defining R¹ include but are not limited to F, Cl, Br, CH₃, CF₃, CH₂CH₃, CH(CH₃)₂, OCH₃, OCF₃, OCH₂CH₃, NH₂, N(H)alkyl (e.g., N(H)CH₃), and CN. In one example of a preferred embodiment wherein Y is moiety ii and a is 1, 2 or 3, each R¹ is the same or different and is independently selected from F, Cl, CH₃, CF₃, OCH₃, and OCF₃ or any subset thereof. In one preferred embodiment, a is 1 or 2 and each R¹ is F.

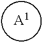

in moiety ii is referred to herein as “Ring A¹.” Ring A¹ is selected from cycloalkyl, phenyl, and 5-10 membered heteroaryl having 1 or 2 heteroatoms selected from N, O and S. Ring A¹ may be bonded to the methylene (when b is 1) or —C(W¹)— through any suitable carbon or heteroatom of Ring A¹. In one embodiment, Ring A¹ is selected from cycloalkyl, phenyl, and 5-6 membered heteroaryl having 1 or 2 heteroatoms selected from N, O and S. In one particular embodiment, Ring A¹ is selected from C₃₋₆cycloalkyl, phenyl, and 5-6 membered N-heteroaryl having 1 or 2 heteroatoms selected from N, O and S. In one embodiment, Ring A¹ is phenyl or 5-6 membered heteroaryl having 1 or 2 heteroatoms selected from N, O and S. In another particular embodiment, Ring A¹ is a 5-6 membered N-heteroaryl optionally having 1 additional heteroatom selected from N, O and S.

Specific examples of groups defining Ring A¹ include but are not limited to cyclopropyl, cyclobutyl, cyclopentyl, cyclohexyl, furanyl, thiophenyl, pyrrolyl, oxazolyl, isoxazolyl, thiazolyl, isothiazolyl, imidazolyl, pyrazolyl, triazolyl, phenyl, pyridinyl, pyridazinyl, pyrimidinyl, pyrazinyl, benzofuranyl, benzothiophenyl, benzimidazolyl, and benzodioxanyl, or any subset thereof. In one embodiment, Ring A¹ is selected from cyclohexyl, phenyl, furanyl, pyrrolyl, pyrazolyl, isoxazolyl, pyridinyl, benzofuranyl and benzodioxanyl, or any subset thereof. In one embodiment, Ring A¹ is selected from cyclohexyl, phenyl, furanyl, pyrrolyl, pyrazolyl, isoxazolyl, and pyridinyl, or any subset thereof. In one particular embodiment, Ring A¹ is selected from cyclohexyl, cyclopropyl, thiophenyl, pyrrolyl, phenyl, and pyridinyl or any subset thereof.

In one specific embodiment, Ring A¹ is cyclohexyl. In one particular embodiment, Ring A¹ is thiophenyl. In another particular embodiment, Ring A¹ is pyridinyl. In one preferred embodiment, Ring A¹ is phenyl.

One particular embodiment of the compounds of the invention is illustrated by formula (I-ii-1):

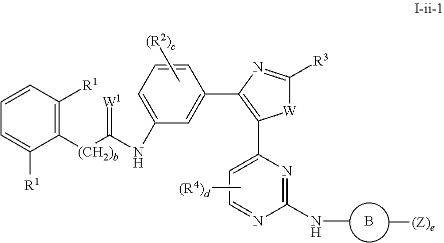

wherein all variables are as defined herein.

One preferred embodiment of such compounds of the invention have the structural formula (I-ii-1) wherein b is 0, W¹ is 0 and both R¹ are F.

Another particular embodiment includes compounds of formula (I-ii-2)

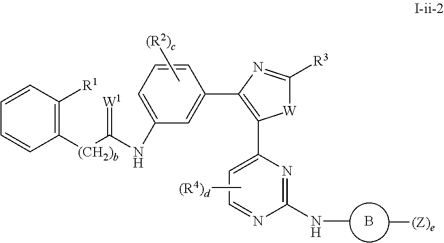

wherein all variables are as defined above.

One preferred embodiment of such compounds of the invention have the structural formula (I-ii-2) wherein b is 0, W¹ is O and R¹ is F.

Another particular embodiment includes compounds of formula (I-ii-3)

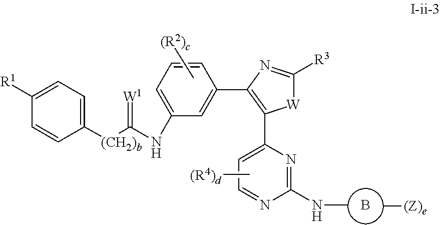

wherein all variables are as defined herein. In one particular embodiment illustrated by formula (I-ii-3), R¹ is F.

Other particular embodiments of the compounds of the invention include:

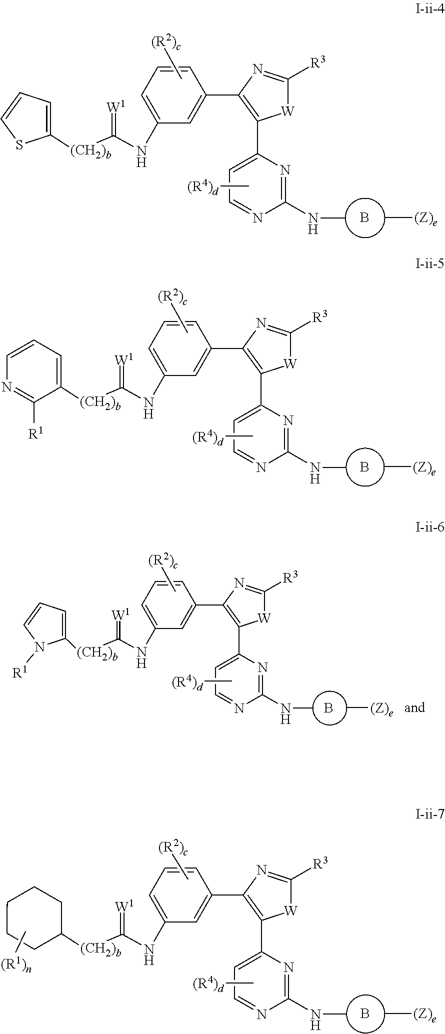

wherein all variables are as defined herein.

Considering compounds of the invention wherein Y is moiety iii, four specific embodiments of compounds of formula (I-iii) above may be illustrated as formulas (I-iii-a), (I-iii-b), (I-iii-c), and (I-iii-d):

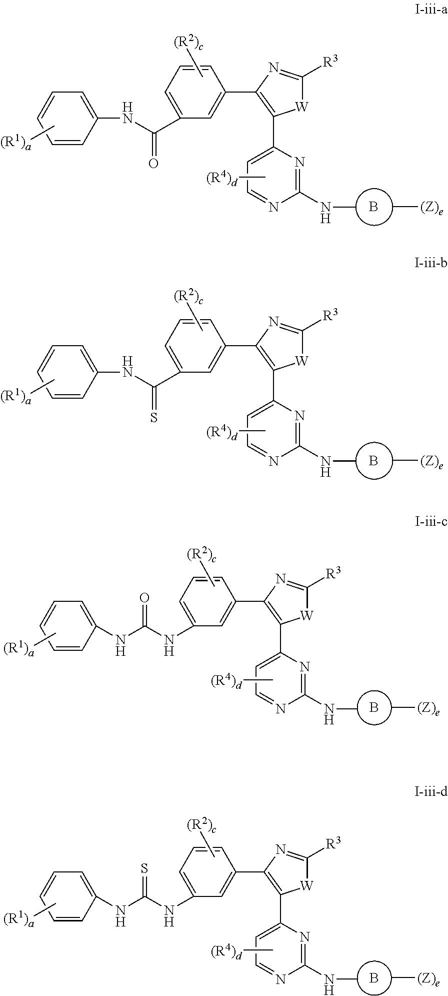

wherein all variables are as defined herein.

Compounds of the invention defined by formulas (I-iii-a) and (I-iii-c), i.e., wherein W¹ of moiety iii is 0, are generally preferred over compounds of formulas (I-iii-b) and (I-iii-d). As between the compounds of the invention defined by formulas (I-iii-a) and (I-iii-c), compounds of formula (I-iii-a) are generally preferred.

For compounds of the invention wherein Y is moiety iii (i.e., compounds defined by formula (I-iii) including formulas (I-iii-a), (I-iii-b), (I-iii-c) and (I-iii-d), particular embodiments are defined wherein a of (R¹)ₐ is 0, 1 or 2. More particular embodiments are defined wherein a is 0 or 2. In one specific embodiment, a is 0. In another specific embodiment, a is 1. In a preferred embodiment, a is 2. In one preferred embodiment, the compounds of the invention are defined by formula (I-iii-a), and a is 2.

In the embodiments, wherein Y is moiety iii and a is any of 1, 2 or 3, the particular and preferred embodiments of R¹ are the same as described above for compounds wherein Y is moiety ii.

One particular embodiment of the compounds of the invention is illustrated by formula (I-iii-1):

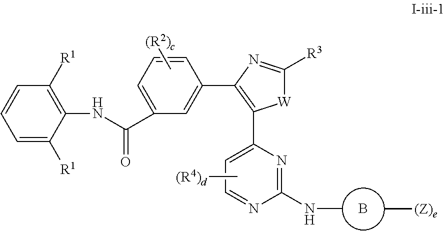

wherein all variables are as defined herein.

One preferred embodiment of such compounds of the invention have the structural formula (I-iii-1) wherein Q² is a bond, W¹ is 0, a is 2 and both R¹ are F.

Referring now to all compounds of the invention (including compounds of formula (I) and all subgeneric formulas described herein), in the compounds of the invention, c is 0, 1 or 2. In particular embodiments, c is 0 or 1, more particularly 0. In embodiments wherein c is 0, the inner phenyl is unsubstituted. For ease of reference, the phenyl ring which is optionally substituted as indicated by (R²)_{c} in formula (I) may be referred to herein from time to time as the “inner phenyl”.

In certain embodiments wherein c is 1 or 2, each R² is the same or different and is independently selected from halo, alkyl, —OR⁶, —NR⁶R⁷ and —CN, or any subset thereof. In one particular embodiment, each R² is the same or different and is independently selected from halo, alkyl, —OR⁶ and —CN, or any subset thereof. In one particular embodiment, when c 1 or 2, each R² is the same or different and is independently selected from halo and alkyl; more preferably halo, particularly F or Cl. In one example of a preferred embodiment, each R² is the same or different and is independently selected from halo (particularly F or Cl), alkyl (particularly methyl), or —O-alkyl (particularly —OCH₃), or any subset thereof.

In one preferred embodiment the compounds of the invention are defined wherein W is S. In another embodiment, compounds of the invention are defined wherein W is O.

In one embodiment, R³ is selected from H, alkyl, haloalkyl, Ph, Het, —R⁵—OR⁶, —R⁵—S(O)_{f}R⁶, —R⁵—S(O)₂—NR⁶R⁷, —NR⁶R⁷, —N(R⁶)cycloalkyl, —N(R⁶)Het, —N(R⁶)R⁵—Het, —N(R⁶)—R⁵—OR⁷, —N(R⁶)—R⁵—NR⁶R⁷, —N(H)C(O)R⁶, —N(H)SO₂R⁶, and —N(R⁶)—R⁵—S(O)_{f}R⁷, or any subset thereof. In one particular embodiment the compounds of formula (I) are defined wherein R³ is selected from H, alkyl, haloalkyl, Het, —R⁵—OR⁶, —R⁵—S(O)_{f}R⁶, —NR⁶R⁷, —N(R⁶)cycloalkyl, —N(R⁶)Het, —N(R⁶)R⁵—Het, —N(R⁶)—R⁵—OR⁷, and —N(R⁶)—R⁵—S(O)_{f}R⁷. In one embodiment R³ is selected from H, alkyl, Het, —R⁵—OR⁶, —R⁵—S(O)_{f}R⁶, —NR⁶R⁷, —N(R⁶)cycloalkyl, —N(R⁶)Het, —N(R⁶)R⁵—Het and —N(R⁶)—R⁵—OR⁷. In one embodiment R³ is H. In one embodiment R³ is —NR⁶R⁷, —N(R⁶)cycloalkyl, or —N(R⁶)Het, where R⁶ is preferably H. In one embodiment, R³ is selected from alkyl, —R⁵—OR⁶, and —R⁵—S(O)_{f}R⁶. In one example of a preferred embodiment, R³ is —N(H)alkyl, —N(C₁₋₃alkyl)₂ or —N(H)C₃₋₆cycloalkyl, or any subset thereof. In another example of a preferred embodiment, R³ is alkyl, and particularly branched alkyl (particularly methyl, ethyl, isopropyl, or tert-butyl).

Ph (as employed in the definition of R³) refers to phenyl optionally substituted 1, 2 or 3 times with a substituent selected from halo, C₁₋₃alkyl, C₁₋₃alkylene-O—C₁₋₃alkyl, OH, C₁₋₃alkylene-OH, NH₂, N(H)C₁₋₃alkyl, N(C₁₋₃alkyl)₂, CN and NO₂, or any subset thereof. In all embodiments wherein Ph is substituted with 2 or 3 substituents, the substituents may be the same or different and are each independently selected from the foregoing list.

Het (as employed in the definition of R³ and in groups defining R³, e.g., —R⁵Het, —N(R⁶)Het, etc.) refers to a 4-6 membered heterocycle having 1 or 2 heteroatoms selected from N, O and S and optionally substituted with 1, 2 or 3 substituents which are the same or different and are each independently selected from halo, C₁₋₃alkyl, haloC₁₋₃alkyl, O—C₁₋₃alkyl, C₁₋₃alkylene-O—C₁₋₃alkyl, OH, C₁₋₃alkylene-OH, oxo, C(O)(C₁₋₃alkyl), C(O)NH₂, C(O)N(C₁₋₃alkyl)₂, SO₃(H), SO₂(C₁₋₃alkyl), C₁₋₃alkylene-SO₃(H), C₁₋₃alkylene-SO₂(C₁₋₃alkyl), NH₂, N(H)C₁₋₃alkyl, N(C₁₋₃alkyl)₂, CN, —CH₂CN, and NO₂, or any subset thereof. In one embodiment, Het in the definition of R³ and in groups defining R³ is a 4-6 membered N-heterocycle optionally having 1 additional heteroatom selected from N, O and S and optionally substituted as described above. In one particular embodiment, Het is a 4-6 membered N-heterocycle having no additional heteroatoms and optionally 1 substituent as described above. In one embodiment, Het is selected from optionally substituted morpholinyl, pyrrolidinyl, piperidinyl, azetidinyl, piperzinyl, thiomorpholinyl, or any subset thereof, wherein the optional substituents are as recited above. In one particular embodiment, Het in the definition of R³ and in groups defining R³, is pyrrolidinyl. In one particular embodiment, R³ is pyrrolidine.

In one embodiment, d is 0 or 1. In one particular embodiment, d is 1. In a preferred embodiment, d is 0.

In certain embodiments wherein d is 1 or 2, each R⁴ is the same or different and is independently selected from halo, alkyl, —OR⁶, —NR⁶R⁷ and —CN, or any subset thereof. In one particular embodiment, each R⁴ is the same or different and is independently selected from halo, alkyl, —OR⁶ and —CN, or any subset thereof. In one particular embodiment, each R⁴ is the same or different and is independently selected from halo and alkyl; more preferably alkyl. In one example of a preferred embodiment, each R⁴ is alkyl (particularly methyl).

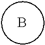

in formula (I) is referred to herein as “Ring B.” Ring B is selected from phenyl, 9-10 membered aryl (particularly bicyclic fused), 5-6 membered heteroaryl and 9-10 membered heteroaryl (particularly bicyclic fused), each heteroaryl having 1, 2 or 3 heteroatoms selected from N, O and S. Ring B may be bonded to —N(H)— through any suitable carbon or heteroatom of Ring B. Specific groups defining Ring B include but are not limited to furanyl, thiophenyl, pyrrolyl, oxazolyl, isoxazolyl, thiazolyl, isothiazolyl, imidazolyl, pyrazolyl, triazolyl, phenyl, pyridinyl, pyridazinyl, pyrimidinyl, pyrazinyl, naphthyl, dihydronaphthyl, indenyl, dihydroindenyl, benzofuranyl, benzothiophenyl, indolyl, isoindolyl, indolinyl, indazolyl, benzimidazolyl, quinolinyl, isoquinolinyl, tetrahydroisoquinolinyl, quinazolinyl, quinoxalinyl, benzopiperidinyl, benzopiperazinyl, benzomorpholinyl, benzotriazolyl, benzopyranyl, chroman, isochroman, benzodioxanyl, and benzodioxolanyl, or any subset thereof.

In one example of a preferred embodiment, Ring B is phenyl.

In another embodiment, Ring B is a 5-6 membered heteroaryl having 1 or 2 heteroatoms selected from N, O and S. In one preferred embodiment, Ring B is a 5-6 membered N-heteroaryl optionally having 1 additional heteroatom which is selected from N, O and S.

Specific examples wherein Ring B is a 5-6 membered heteroaryl include furanyl, pyrrolyl, pyrazolyl, thiophenyl, isoxazolyl, pyridinyl, pyrimidinyl and pyrazinyl. In one specific example of a preferred embodiment, Ring B is pyridinyl.

In another embodiment, Ring B is 9-10 membered aryl, particularly indenyl or naphthyl.

In another embodiment, Ring B is a 9-10 membered bicyclic fused heteroaryl having 1, 2 or 3 heteroatoms selected from N, O and S, more particularly 1 or 2 heteroatoms selected from N, O and S. In one particular embodiment, Ring B is a 9-10 membered bicyclic fused N-heteroaryl optionally having 1 additional heteroatom which is selected from N, O and S. Specific examples wherein Ring B is a 9-10 membered bicyclic fused heteroaryl include benzopiperidinyl, benzomorpholinyl, benzofuranyl and benzodioxanyl. In one specific example of a particular embodiment, Ring B is benzopiperidinyl or benzomorpholinyl.

The substituents Z may be bound to Ring B through any available carbon or heteroatom of Ring B. However, the moiety Z should be understood to be defined in view of the definition of Ring B so as to avoid embodiments which the organic chemist of ordinary skill would consider to be obviously unstable or inoperative. For example, in those embodiments wherein Ring B is a heteroaryl, e is not 0, and Z is bound to a heteroatom of Ring B; then Z is defined as a moiety which is bound to Ring B through either carbon or a heteroatom suitable for binding to the heteroatom of Ring B. Thus for example, when Ring B is a heteroaryl and Z is bound to a N of Ring B, then Z is defined as a moiety capable of binding to the N of Ring B; accordingly in such embodiment, Z may not for example, be halo. Z moieties suitable for binding to a N of Ring B will be apparent to those skilled in the art and include but are not limited to H, alkyl (e.g., N-methyl) and oxo (e.g., N-oxide).

When Ring B is selected from phenyl and 5-6 membered heteroaryl, e is 0, 1, 2 or 3. When e is 0, Ring B is unsubstituted. In one embodiment wherein Ring B is phenyl or 5-6 membered heteroaryl, e is 1, 2 or 3. In the embodiment, wherein Ring B is phenyl, e is generally 1, 2 or 3, more particularly, 1 or 2, most particularly 2. In embodiments wherein Ring B is a 5-6 membered heteroaryl such as pyridinyl, e is particularly 1 or 2, more particularly 1.

In the embodiments wherein Ring B is phenyl or 5-6 membered heteroaryl and e is 1, 2 or 3, each Z is the same or different and is independently selected from:
halo, alkyl, haloalkyl, alkenyl,
Het², —R⁵Het², Het³-Het²,
oxo, —OR⁶, —R⁵—OR⁶, —O—R⁵—OR⁶, —OHet², —O—R⁵—Het², —O—R⁵—NR⁶R⁷,
—O—R⁵—S(O)₂R⁶, —C(O)NR⁶R⁷, —R⁵—C(O)NR⁶R⁷, —CO₂R⁶, —R⁵—CO₂R⁶,
—S(O)_{f}R⁶ (e.g., —S(O)₂R⁶), —R⁵—S(O)₂R⁶, —S(O)_{f}Het², —R⁵—S(O)₂Het², —S(O)₂NR⁶R⁷,
—R⁵—S(O)₂NR⁶R⁷, —S(O)₂—R⁵—NR⁶R⁷,
—NR⁶R⁷, —R⁵—NR⁶R⁷, —N(R⁶)Het², —N(R⁶)—R⁵cycloalkyl, —N(R⁶)—R⁵—Het²,
   —N(R⁶)—R⁵—OR⁷, —N(R⁶)—R⁵—S(O)_{f}R⁷, —N(R⁶)—R⁵—CN, —N(R⁶)—R⁵—NR⁶R⁷,
   —N(H)S(O)₂R⁶, —N(R⁶)—C(O)—NR⁶R⁷, —N(R⁶)—S(O)₂—NR⁶R⁷,
—CN, —R⁵—CN and —NO₂.

In one embodiment, each Z is the same or different and is independently selected from:
halo, alkyl, haloalkyl,
Het², —R⁵Het², Het³-Het²,
oxo, —OR⁶, —R⁵—OR⁶, —O—R⁵—OR⁶, —OHet², —O—R⁵—Het², —O—R⁵—NR⁶R⁷,
—S(O)_{f}R⁶ (e.g., —S(O)₂R⁶), —R⁵—S(O)₂R⁶, —R⁵—S(O)₂Het², —S(O)₂NR⁶R⁷,
—R⁵—S(O)₂NR⁶R⁷,
—NR⁶R⁷, —R⁵—NR⁶R⁷, —N(R⁶)Het², —N(R⁶)—R⁵—OR⁷, —N(R⁶)—R⁵—S(O)_{f}R⁷,
—N(R⁶)—R⁵—NR⁶R⁷, —CN, and —R⁵—CN, or any subset thereof.

Het² and Het³ in this particular embodiment may each be optionally substituted.

In one particular embodiment, each Z is the same or different and is independently selected from:
halo, alkyl, haloalkyl,
Het², —R⁵Het², Het³-Het²,
oxo, —OR⁶, —O—R⁵—OR⁶, —OHet², —O—R⁵—Het², —O—R⁵—NR⁶R⁷,
—R⁵—S(O)₂R⁶, —R⁵—S(O)₂NR⁶R⁷,
—NR⁶R⁷, —R⁵—NR⁶R⁷, —N(R⁶)—R⁵—NR⁶R⁷, and —CN, or any subset thereof.

Het² and Het³ in this particular embodiment may each be optionally substituted.

In one particular embodiment wherein e is 2, one Z is at the meta position and is halo, particularly F. For illustration, the following example shows one Z group bound at the meta position wherein Ring B is phenyl, e′ is 1, and the remaining Z group and all other variables are as defined herein.

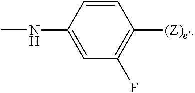

The embodiment may be further illustrated when Ring B is pyridinyl as follows:

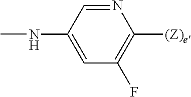

wherein e′ is 1 and the remaining Z group and all other variables are as defined herein.

In another particular embodiment wherein Ring B is phenyl and e is 1, Z is at the meta position, illustrated as follows.

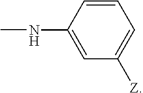

In a particular embodiment wherein Ring B is pyridinyl and e is 1, Z is at the para position, illustrated as follows.

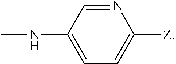

In one particular embodiment, Ring B is phenyl or 5-6 membered heteroaryl having 1, 2, or 3 heteroatoms selected from N, O and S, e is 1, 2 or 3, particularly 1 or 2 and each Z is the same or different and is independently selected from halo, alkyl, Het², —R⁵—Het², oxo, —OR⁶, —OHet², —O—R⁵—Het² and —O—R⁵—NR⁶R⁷, or any subset thereof. Het² and Het³ in this embodiment are optionally substituted.

In one embodiment wherein Ring B is phenyl, e is 2, one Z is halo and the other Z is selected from —OHet², —O—R⁵—Het² and —O—R⁵—NR⁶R⁷. Het² in this embodiment is optionally substituted. In one particular variation of this embodiment, the halo is in the meta position and the remaining Z is in the para position.

In one embodiment wherein Ring B is phenyl, e is 2, one Z is F, and the other Z is Het².

Het² in this embodiment is optionally substituted. One specific example of this embodiment may be illustrated as follows:

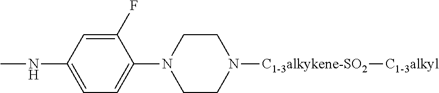

wherein Het² in this example is substituted piperazine and all variables are as defined herein.

In one embodiment wherein Ring B is phenyl, e is 2, one Z is F, and the other Z is —O—R⁵—Het² or —O—R⁵—NR⁶R⁷. Het² in this embodiment is optionally substituted. One specific example of each of these embodiments may be illustrated as follows:

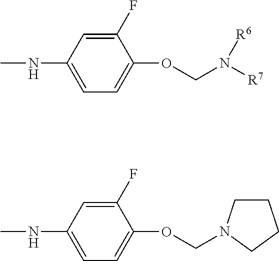

wherein Het² in the illustrated example is unsubstituted pyrrolidine and all variables are as defined herein.

In one embodiment wherein Ring B is phenyl, e is 1, and Z is —R⁵—Het². Het² in this embodiment is optionally substituted. One specific example of this embodiment may be illustrated as follows:

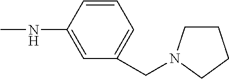

wherein Het² in the illustrated example is unsubstituted pyrrolidine and all variables are as defined herein.

In one embodiment wherein Ring B is 5-6 membered heteroaryl (e.g., pyridinyl), e is 1, and Z is Het². Het² in this embodiment is optionally substituted. An example of this embodiment may be illustrated as follows:

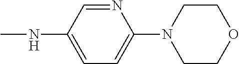

wherein Het² in the illustrated example is unsubstituted morpholine and all variables are as defined herein.

In one embodiment wherein Ring B is 5-6 membered heteroaryl (e.g., pyridinyl), e is 1 and Z is selected from Het², —O—R⁵—OR⁶, —OHet², —O—R⁵—Het², —O—R⁵—NR⁶R⁷. Het² in this embodiment is optionally substituted. In one embodiment wherein Ring B is 5-6 membered heteroaryl (e.g., pyridinyl), e is 1 and Z is Het² or —O—R⁵—Het², wherein Het² is optionally substituted.

When Ring B is a 9-10 membered aryl or heteroaryl (typically a bicyclic fused aryl or heteroaryl), e is 0, 1 or 2 and each Z is the same or different and is independently selected from halo, alkyl, oxo, —OR⁶ and —NR⁶R⁷. In one embodiment, wherein Ring B is bicyclic fused 9-10 membered aryl or heteroaryl, e is 0 or 1. In one particular embodiment, e is 0 and hence, Ring B is unsubstituted. In one embodiment, Ring B is a 9-10 membered bicyclic fused aryl or heteroaryl, e is 1 and Z is halo, C₁₋₃alkyl, oxo or —OC₁₋₃alkyl. In one embodiment wherein Ring B is 9-10 membered bicyclic fused aryl or heteroaryl, e is 1 and Z is selected from alkyl and oxo, such as methyl, ethyl, isopropyl, N-methyl, N-ethyl, oxo and N-oxide.

Following is a list of specific examples of groups defining Z. In the following specific examples, R⁵ refers to C₁₋₄alkylene, however in certain examples below, it will be appreciated that R⁵ should be understood to be C₂₋₄alkylene in order to avoid unstable species. Specific examples of groups defining Z include but are not limited to:

halo (e.g., F or Cl);
alkyl (e.g., CH₃),
haloalkyl (e.g., CF₃),
Het² (e.g., 5-6 membered heterocycle or 5-6 membered heteroaryl having 1 or 2 heteroatoms selected from N, O and S and substituted variants thereof)
R⁵—Het² (e.g., alkyl-morpholine, alkyl-piperidine, alkyl-pyrrolidine, and substituted variants thereof),
   Het³-Het² (e.g., piperidinyl-piperidine and substituted variants thereof),
   oxo (e.g., N-oxide, carbonyl or sulfonyl),

### OR⁶ (e.g., OH, OCH₃),

### R⁵—OR⁶ (e.g., (CH₂)₂—OCH₃),

### O—R⁵—OR⁶ (e.g., O—(CH₂)₂—OCH₃),

OHet² (e.g., O-piperidine, O-pyrrolidine and substituted variants thereof)
O—R⁵—Het² (e.g., O—(CH₂)-2-morpholine, O—(CH₂)₂-pyrrolidine, O—(CH₂)₂-piperidine and substituted variants thereof),

### O—R⁵—NR⁶R⁷ (e.g., O—(CH₂)₂—NH₂,

O—(CH₂)₂—N(H)CH₃,
O—(CH₂)₂—N(CH₃)₂, O—(CH₂)₂—N(CH₂CH₃)₂, O—(CH₂)₃—NH₂,
O—(CH₂)₃—N(H)CH₃, O—(CH₂)₃—N(CH₃)₂, O—(CH₂)₃—N(CH₂CH₃)₂,
O—CH₂CH(CH₃)—NH₂, O—CH₂CH(CH₃)—N(H)CH₃, O—CH₂CH(CH₃)—N(CH₃)₂,
O—CH₂CH(CH₃)—N(CH₂CH₃)₂,

### C(O)NR⁶R⁷ (e.g., C(O)NH₂),

S(O)_{f}R⁶, particularly SO₂R⁶ (e.g., SO₂CH₃, SO₂CH₂CH₃),
R⁵—SO₂R⁶ (e.g., CH₂—SO₂CH₃, CH₂—SO₂CH₂CH₃, (CH₂)₂—SO₂CH₃ and

### (CH₂)₂—SO₂CH₂CH₃),

### SO₂NR⁶R⁷ (e.g., SO₂NH₂, SO₂N(H)CH₃)

### R⁵—SO₂NR⁶R⁷ (e.g., (CH₂)₂—SO₂NH₂),

### R⁵—NR⁶R⁷ (e.g., —(CH₂)₃—N(CH₃)₂),

N(H)Het² (e.g., N(H)-piperidine, N(H)-piperazine and substituted variants thereof),
N(H)—R⁵—Het² (e.g., NH—(CH₂)₃-piperazine-N-methyl),

### N(CH₃)—R⁵—OR⁷ (e.g., N(CH₃)—(CH₂)₃—OCH₃),

### N(H)—R⁵—NR⁶R⁷ (e.g., NH—(CH₂)₃—N(CH₃)₂),

### N(CH₃)—R⁵—NR⁶R⁷ (e.g., N(CH₃)—(CH₂)₃—N(CH₃)₂),

### CN, and R⁵—CN (e.g., (CH₂)₂CN),

or any subset thereof.

In the definition of Z, each Het² is the same or different and is independently heterocycle heteroaryl, said heterocycle or heteroaryl having 1 or 2 heteroatoms selected from N, O and S and optionally substituted with 1, 2 or 3 substituents selected from:
halo, C₁₋₃alkyl, haloC₁₋₃alkyl, C₁₋₃alkylene-O—C₁₋₃alkyl, OH,
C₁₋₃alkylene-OH, oxo, C(O)(C₁₋₃alkyl), C(O)₂—C₁₋₃alkyl,
C(O)—(C₁₋₃alkylene)-O(C₁₋₃alkyl), C(O)₂-benzyl, SO₃H, SO₂(C₁₋₃alkyl),
C₁₋₃alkylene-SO₃H, C₁₋₃alkylene-SO₂(C₁₋₃alkyl), NH₂, N(H)C₁₋₃alkyl, N(C₁₋₃alkyl)₂, CN and C₁₋₃alkylene-CN.

Het² includes 4-10 membered heterocycles and spiro systems having from 7 to 12 membered spiro systems, wherein the heterocycles and spirosystems include 1, 2, or 3 heteroatoms selected from N, O and S. In one embodiment, Het² is not a 7-12 membered spiro system. In all embodiments wherein Het² is substituted with 2 or 3 substituents, the substituents are the same or different and are each independently selected from the foregoing list.

In one embodiment, Het² is independently a heterocycle or 5-6 membered heteroaryl, said heterocycle or heteroaryl having 1 or 2 heteroatoms selected from N, O and S and optionally substituted 1, 2 or 3 times with a substituent selected from the foregoing.

In one particular embodiment, Het² is independently a heterocycle having 1 or 2 heteroatoms selected from N, O and S and optionally substituted 1, 2 or 3 times with a substituent selected from the foregoing.

In one embodiment, Het² is independently a 5-6 membered heteroaryl having 1 or 2 heteroatoms selected from N, O and S and optionally substituted 1, 2 or 3 times with a substituent selected from the foregoing.

In one more particular embodiment, Het² is a 5-6 membered heterocycle having 1 or 2 heteroatoms selected from N, O and S and optionally substituted 1, 2 or 3 times with a substituent defined above.

In one particular embodiment, Het² is a 5-6 membered N-heterocycle optionally having 1 additional heteroatom selected from N, O and S and optionally substituted 1 or 2 times with a substituent defined above.

In certain embodiments of the invention, the group Het² is unsubstituted. In those embodiments wherein Het² is substituted, a particular embodiment is defined wherein the substituent(s) is(are) selected from
C₁₋₃alkyl, haloC₁₋₃alkyl, O—C₁₋₃alkyl, C₁₋₃alkylene-OH, oxo, C(O)(C₁₋₃alkyl),
C(O)₂—C₁₋₃alkyl, C(O)—(C₁₋₃alkylene)-O(C₁₋₃alkyl), C₁₋₃alkylene-SO₃(H),
C₁₋₃alkylene-SO₂(C₁₋₃alkyl), NH₂, N(H)C₁₋₃alkyl, N(C₁₋₃alkyl)₂, and
C₁₋₃alkylene-CN, or any subset thereof.

In a more particular embodiment, the optional substituent on Het² is selected from
C₁₋₃alkyl, haloC₁₋₃alkyl, C₁₋₃alkylene-OH, oxo,
C(O)(C₁₋₃alkyl), C₁₋₃alkylene-SO₃(H), C₁₋₃alkylene-SO₂(C₁₋₃alkyl) and
C₁₋₃alkylene-CN, or any subset thereof.

Specific examples of groups defining Het² within the definition of Z include but are not limited to:

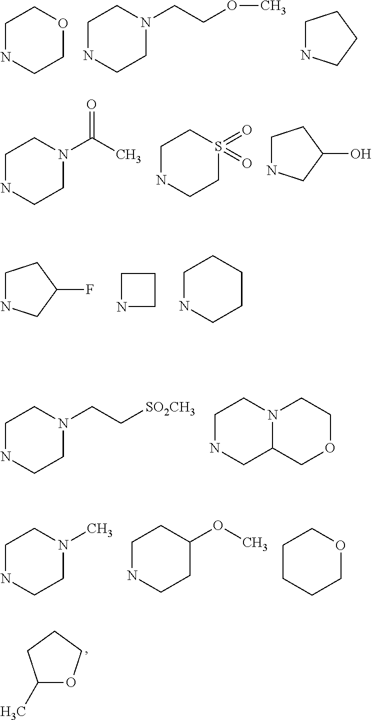

or any subset thereof.

Het³ in the definition of Z is employed in the group Het³-Het². Het³ is a 4-7 membered heterocycle or 5-7 membered heteroaryl, said heterocycle or heteroaryl having 1 or 2 heteroatoms selected from N, O and S and optionally substituted with 1 or 2 substituents selected from halo, C₁₋₃alkyl, haloC₁₋₃alkyl and O—C₁₋₃alkyl. It should be understood that reference to substituents on Het³ refers to optional substituents in addition to Het². In all embodiments wherein Het³ is substituted with 2 substituents, the substituents are the same or different and are each independently selected from the foregoing list.

In one embodiment, Het³ is a 5-6 membered heterocycle or 5-6 membered heteroaryl, said heterocycle or heteroaryl having 1 or 2 heteroatoms selected from N, O and S and optionally substituted 1 or 2 times, in addition to Het², with halo, C₁₋₃alkyl, haloC₁₋₃alkyl or O—C₁₋₃alkyl.

More particularly, in one embodiment, Het³ in the definition of Z is a 5-6 membered heterocycle having 1 or 2 heteroatoms selected from N, O and S and optionally substituted 1 or 2 times, in addition to Het², with a substituent selected from the foregoing.

In one embodiment of the invention, Het³ in the definition of Z is unsubstituted, except by Het².

In one embodiment, the compounds of the invention are defined wherein R⁶ and R⁷ are the same or different and are each independently selected from H, C₁₋₃alkyl and haloC₁₋₃alkyl, or any subset thereof.

It is to be understood that the present invention includes all combinations and subsets of the particular groups defined hereinabove.

Specific examples of compounds of the present invention include those recited in the Examples which follow as well as pharmaceutically acceptable salts of compounds exemplified as the free base and free base versions and other pharmaceutically acceptable salts of those compounds exemplified as salts.

Preferred compounds of the invention are selected from:
N-{3-[2-Amino-5-(2-{[3-{[2-(dimethylamino)ethyl]oxy}-4-(methyloxy)phenyl]amino}-4-pyrimidinyl)-1,3-thiazol-4-yl]phenyl}-2,6-difluoro-N-methylbenzamide;
N-(3-{2-Amino-5-[2-({3-chloro-4-[2-(dimethylamino)ethoxy]-phenyl}amino)pyrimidin-4-yl]-1,3-thiazol-4-yl}phenyl)-2,6-difluorobenzamide-formic acid
N-[3-(2-Amino-5-{2-[(3,4,5-trimethoxyphenyl)amino]pyrimidiN-4-yl}-1,3-thiazol-4-yl)phenyl]-2,6-difluorobenzamide;
N-[3-(2-(Dimethylamino)-5-{2-[(4-{[2-(dimethylamino)ethyl]oxy}phenyl)amino]-4-pyrimidinyl}-1,3-thiazol-4-yl)phenyl]-2,6-difluorobenzamide;
2,6-Difluoro-N-{3-[5-(2-{[4-(methyloxy)-3-(4-methyl-1-piperazinyl)phenyl]amino}-4-pyrimidinyl)-1,3-thiazol-4-yl]phenyl}benzamide;
N-[3-(5-{2-[(4-{[2-(Dimethylamino)ethyl]oxy}-3-fluorophenyl)amino]-4-pyrimidinyl}-2-ethyl-1,3-thiazol-4-yl)phenyl]-2,6-difluorobenzamide; and
N-[3-(5-{2-[(3-Chloro-4-{[2-(1-pyrrolidinyl)ethyl]oxy}phenyl)amino]-4-pyrimidinyl}-2-ethyl-1,3-thiazol-4-yl)phenyl]-2,6-difluorobenzamide;
   and pharmaceutically acceptable salts thereof.

Particular preferred compounds of the invention include but are not limited to:
N-[3-(2-(Dimethylamino)-5-{2-[(4-{[2-(dimethylamino)ethyl]oxy}phenyl)amino]-4-pyrimidinyl}-1,3-thiazol-4-yl)phenyl]-2,6-difluorobenzamide; and
N-[3-(5-{2-[(4-{[2-(Dimethylamino)ethyl]oxy}-3-fluorophenyl)amino]-4-pyrimidinyl}-2-ethyl-1,3-thiazol-4-yl)phenyl]-2,6-difluorobenzamide;
   and pharmaceutically acceptable salts thereof.

It will be appreciated by those skilled in the art that the compounds of formula (I) may be utilized as a pharmaceutically acceptable salt thereof. The pharmaceutically acceptable salts of the compounds of formula (I) include conventional salts formed from pharmaceutically acceptable (i.e., non-toxic) inorganic or organic acids or bases as well as quaternary ammonium salts. Representative salts include the following: acetate, benzenesulfonate, benzoate, bicarbonate, bisulfate, bitartrate, borate, bromide, calcium edetate, camsylate, carbonate, chloride, clavulanate, citrate, dihydrochloride, edetate, edisylate, estolate, esylate, fumarate, gluceptate, gluconate, glutamate, glycollylarsanilate, hexylresorcinate, hydrabamine, hydrobromide, hydrochloride, hydroxynaphthoate, iodide, isethionate, lactate, lactobionate, laurate, malate, maleate, mandelate, mesylate (methanesulphonate), methylbromide, methylnitrate, methylsulfate, monopotassium maleate, mucate, napsylate, nitrate, N-methylglucamine, oxalate, pamoate (embonate), palmitate, pantothenate, phosphate/diphosphate, polygalacturonate, potassium, salicylate, sodium, stearate, subacetate, succinate, tannate, tartrate, teoclate, tosylate (methylbenzenesulfonate), triethiodide, trimethylammonium and valerate. Other salts, such as oxalic, which are not themselves pharmaceutically acceptable, may be useful in the preparation of salts useful as intermediates in obtaining compounds of this invention and these form a further aspect of the invention. In one embodiment, the compound of formula (I) is in the form of the hydrochloride salt, including mono- and di-hydrochloride salts.

Processes for preparing pharmaceutically acceptable salts of compounds such as the compounds of formula (I) are conventional in the art. See, e.g., Burger's Medicinal Chemistry And Drug Discovery 5th Edition, Vol 1: Principles And Practice.

As will be apparent to those skilled in the art, in the processes described below for the preparation of compounds of formula (I), certain intermediates, may be in the form of pharmaceutically acceptable salts of the compound. Processes for preparing pharmaceutically acceptable salts of intermediates are known in the art and are analogous to the processes for preparing pharmaceutically acceptable salts of other compounds such as the compounds of formula (I).

Compounds of the invention are believed to inhibit of one or more kinases and in particular one or more Raf family kinases (“Raf inhibitor”) and/or ErbB family kinases (i.e., EGFR, ErbB2 and ErbB4 (“ErbB inhibitor”). Compounds of the invention may also inhibit one or more other kinases, and particularly other tyrosine kinases. Certain compounds of the invention may inhibit B-Raf (B-Raf inhibitor”). Certain compounds of the invention may inhibit ErbB2 (“ErbB2 inhibitor”). Compounds of the invention may be inhibitors of either Raf family kinases or ErbB family kinases, or in some instances may inhibit both. It is well documented that Raf inhibitors, including B-Raf inhibitors, and ErbB inhibitors, including ErbB2 inhibitors, are believed to be useful as anticancer and antitumor agents. See, e.g., Davies (2002) supra, Garnett (2004) supra, Zebisch (2006) supra, Normanno (2005) supra and Hynes (2005) supra. The anticancer and antitumor effects of these kinase inhibitors is currently believed to result from inhibition of one or more Raf family kinases and/or ErbB family kinases, and the effect of such inhibition on cell lines whose growth and/or viability is dependent on the kinase activity of Raf and/or ErbB family kinases.

Some compounds of the invention may be selective inhibitors of Raf family kinases (“selective Raf inhibitor”), meaning that preferential inhibition of one or more Raf family kinases is significantly greater than that of any number of other kinases, for example by a factor of 5-fold or more. Certain compounds of the invention may be selective inhibitors of ErbB family kinases (“selective ErbB inhibitor”), meaning that preferential inhibition of one or more ErbB family kinases is significantly greater than that of any number of other kinases, for example by a factor of 5-fold or more. Still other compounds of the invention may selectively inhibit Raf family kinases and ErbB family kinases over other kinases (“selective dual Raf/ErbB inhibitor”), meaning that inhibition of one or more Raf family kinases and one or more ErbB family kinases is each significantly greater than that of any number of other kinases, for example by a factor of 5-fold or more.

However, the present invention is not limited to compounds which are selective inhibitors of one or both of Raf family kinases and/or ErbB family kinases; rather, the present invention expressly contemplates that certain compounds of the invention may possess activity against multiple kinases, including kinases other than Raf family kinases and ErbB family kinases. For example, particular compounds of the invention may possess activity against multiple other kinases, including but not limited to IGFR, IR, IRR, Src, VEGFR, PDGFR, Met, Lyn, Lck, Alk5, Aurora A and B, JNK, Syk, p38, BTK, FAK, Abl, Ack1, Arg, BLK, CAMK1δ, CDK6, CK1, cKit, CSK, DDR2, Ephrin receptors, FGFR, Flt3, Fms, Fyn, Hck, HIPK2, Itk, MINK, Mnk2, PAK3, PKCμ, PKD2, PTK5, Ret, Ron, SIK, Tie2, TrkB, Yes, as well. Particular compounds of the invention may be deemed to be unselective or non-selective, meaning that they are not deemed to be selective for any particular kinase over others.

As used herein, a Raf inhibitor is a compound that exhibits a pIC₅₀ of greater than about 6 against at least one Raf family kinase in one or more of the Raf inhibition enzyme assays described below and/or an IC₅₀ of not greater than about 5 μM potency against one cell line that expresses mutated B-Raf kinase (e.g., A375P, Colo205, HT-29, SK-MEL-3, SK-MEL-28) in one or more of the cellular proliferation assays described below. In a particular embodiment, a Raf inhibitor refers to a compound of the invention that exhibits a pIC₅₀ of greater than about 6.5 against at least one Raf family kinase in one or more of the Raf inhibition enzyme assays described below and an IC₅₀ of not greater than about 500 nM potency against at least one cell line that expresses mutated B-Raf kinase in one or more of the cellular proliferation assays described below.

An ErbB inhibitor is a compound which exhibits a pIC₅₀ of greater than about 6 against at least one ErbB family kinase in one or more of the ErbB inhibition enzyme assays described below and/or an IC₅₀ of not greater than about 5 μM potency against at least one cell line (e.g., BT474 or HN5) that overexpresses at least one ErbB family kinase in one or more of the cellular proliferation assays described below. Similarly, in one particular embodiment, a ErbB inhibitor refers to a compound of the invention which exhibits a pIC₅₀ of greater than about 6.5 against at least one ErbB family kinase in one or more of the ErbB inhibition enzyme assays described below and/or an IC₅₀ of not greater than about 500 nM potency against at least one cell line that overexpresses at least one ErbB family kinase in one or more of cellular proliferation assays described below.

A “dual Raf/ErbB inhibitor” refers to a compound of the invention which exhibits a pIC₅₀ of greater than about 6 against at least one. ErbB family kinase and against at least one Raf family kinase in one or more of the enzyme inhibition assays described below and an IC₅₀ of not greater than about 5 μM potency against at least one cell line that overexpresses at least one ErbB family kinase in one or more of the cellular proliferation assays described below and an IC₅₀ of not greater than about 5 μM potency against at least one cell line that expresses mutated B-Raf kinase in one or more of the cellular proliferation assay described below. In one embodiment, a “dual Raf/ErbB inhibitor” refers to a compound of the invention which exhibits a pIC₅₀ of greater than about 6.5 against at least one ErbB family kinase and against at least one Raf family kinase in one or more of the enzyme inhibition assays described below and an IC₅₀ of not greater than about 500 nM potency against at least one cell line that overexpresses at least one ErbB family kinase in one or more of the cellular proliferation assays described below and an IC₅₀ of not greater than about 500 nM potency against at least one cell line that expresses mutated B-Raf kinase in one or more of the cellular proliferation assays described below.

A “B-Raf inhibitor” refers to a compound of the invention that exhibits a pIC₅₀ of greater than about 6.5 against B-Raf in one or more of the Raf inhibition enzyme assays described below and an IC₅₀ of not greater than 500 nM potency against at least one cell line that expresses mutated B-Raf kinase in one or more of the cellular proliferation assays described below. An “ErbB2 inhibitor” refers to a compound of the invention which exhibits a pIC₅₀ of greater than about 6.5 against at least one ErbB family kinase in one or more of the ErbB inhibition enzyme assays described below and/or an IC₅₀ of not greater than about 500 nM potency against at least one cell line that overexpresses at least one ErbB family kinase (e.g., ErbB2) in one or more of the cellular proliferation assays described below. A “dual B-Raf/ErbB2 inhibitor” refers to a compound of the invention which exhibits a pIC₅₀ of greater than about 6.5 against at least one ErbB family kinase and against B-Raf in one or more of the enzyme inhibition assays described below and an IC₅₀ of not greater than about 500 nM potency against at least one cell line that overexpresses at least one ErbB family kinase (e.g., ErbB2) in one or more of the cellular proliferation assays described below and an IC₅₀ of not greater than about 500 nM potency against at least one cell line that expresses mutated B-Raf kinase in one or more of the cellular proliferation assays described below.

The present invention provides compounds for use in medical therapy in a mammal, e.g., a human, in need thereof. The present invention provides methods for the treatment of several conditions in a mammal in need thereof, all of which comprise the step of administering a therapeutically effective amount of a compound of the invention. All methods described herein are applicable to mammals, and particularly to humans. As used herein, the term “treatment” or “treating” in the context of therapeutic methods, refers to alleviating the specified condition, eliminating or reducing the symptoms of the condition, slowing or eliminating the progression, invasion, or metastatic spread of the condition and preventing or delaying the reoccurrence of the condition in a previously afflicted subject. The present invention further provides use of the compounds of the invention for the preparation of a medicament for the treatment of several conditions in a mammal (e.g., human) in need thereof.

In particular, the present invention provides compounds for use in the treatment of a condition mediated by at least one Raf family kinase (e.g., B-Raf) or at least one ErbB family kinase in a mammal in need thereof. The present invention provides a method for treating a condition mediated by at least one Raf family kinase (e.g., B-Raf) or at least one ErbB family kinase in a mammal (e.g., a human) in need thereof, which method comprises administering to the mammal a therapeutically effective amount of the compound of the invention.

In another embodiment, the invention provides compounds for use in regulating, modulating, binding or inhibiting one or more Raf family kinases (e.g., B-Raf) in a mammal and compounds for use in regulating, modulating, binding or inhibiting one or more ErbB family kinases (e.g., ErbB2) in a mammal. The invention also provides methods for regulating, modulating, binding, or inhibiting at least one Raf family kinase (e.g., B-Raf) in a mammal and methods for regulating, modulating, binding, or inhibiting at least one ErbB family kinases (e.g., ErbB2) in a mammal, each method comprising administering a therapeutically effective amount of a compound of the invention. “Regulating, modulating, binding or inhibiting at least one Raf family kinase” refers to regulating, modulating, binding or inhibiting the activity of at least one Raf family kinase, as well as regulating, modulating, binding or inhibiting overexpression of an upstream regulator of at least one Raf family kinase in order to inhibit the cellular potency of its signaling ability. “Regulating, modulating, binding or inhibiting at least one ErbB family kinase” refers to regulating, modulating, binding or inhibiting the activity of at least one ErbB family kinase, as well as regulating, modulating, binding or inhibiting overexpression of an upstream regulator of at least one ErbB family kinase in order to inhibit the cellular potency of its signaling ability.

In a particular embodiment, the invention provides compounds for use in the treatment of a condition mediated by inappropriate activity of one or more Raf family kinases (e.g., B-Raf), or an upstream activator of one or more Raf family kinases, or inappropriate activity of one or more ErbB family kinases or an upstream activator of one or more ErbB family kinases in a mammal. The invention further provides methods for the treatment of a condition mediated by inappropriate activity of one or more Raf family kinases (particularly B-Raf) or ErbB family kinases (particularly ErbB2), in a mammal in need thereof, comprising administering to the mammal, a therapeutically effective amount of a compound of the invention. In an additional aspect, the present invention provides the use of a compound of the invention for the preparation of a medicament for the treatment of a condition mediated by inappropriate activity of one or more Raf family kinases (particularly B-Raf) or ErbB family kinases (particularly ErbB2), in a mammal. One example of a condition mediated by inappropriate activity of one or more Raf family kinases or ErbB family kinases includes neoplasms.

By “inappropriate activity” is meant Raf family kinase or ErbB family kinase activity that deviates from the expected activity for that kinase or for an upstream activator of that kinase in a particular mammal. The inappropriate activity of a Raf family kinase may arise from one or more of A-Raf, B-Raf or c-Raf or an upstream activator of a Raf family kinase. The inappropriate activity of an ErbB family kinase may arise from one or more of EGFR, ErbB2 or ErbB4 or an upstream activator of an ErbB family kinase. Inappropriate Raf family kinase or ErbB family kinase activity may take the form of, for instance, an abnormal increase in activity, or an aberration in the timing and/or control of Raf or ErbB family kinase activity, respectively. Such inappropriate activity may result, for example, from overexpression or mutation of the protein kinase, upstream activator, receptor or ligand leading to inappropriate or uncontrolled activation of the corresponding kinase or receptor. Furthermore, it is also contemplated that unwanted Raf family kinase or ErbB family kinase activity may reside in an abnormal source, such as a neoplasm. Thus, the level of Raf family kinase or ErbB family kinase activity does not need to be abnormal to be considered inappropriate in the case where the activity derives from an abnormal source including, but not limited to, upstream activators (e.g., activated mutant Ras GTPases) or neoplasm. In one example of inappropriate Raf family activity not resulting from mutation or overexpression of a Raf family kinase, inappropriate activity of a Ras GTPase may result from mutation or overexpression of said Ras GTPase, for example the G13D mutation in KRas2, and may lead to overactivation of the MAPK pathway mediated by Raf family activity.

Thus, in one embodiment, the present invention provides compounds for use in the treatment of a condition which directly or indirectly results from a mutation of one or more Raf or ErbB family kinases (particularly B-Raf or ErbB2) or overexpression of one or more Raf or ErbB family kinases (particularly B-Raf or ErbB2), or a mutation of an upstream activator of one or more Raf or ErbB family kinases, or overexpression of an upstream activator of one or more Raf or ErbB family kinases in a mammal in need thereof. The present invention provides methods for the treatment of a condition which directly or indirectly results from mutation of a Raf or ErbB family kinase or overexpression of a Raf or ErbB family kinase, or a mutation of an upstream activator of a Raf or ErbB family kinase or overexpression of an upstream activator of a Raf or ErbB family kinase in a mammal in need thereof, comprising administering to the mammal a therapeutically effective amount of a compound of the invention. In an additional aspect, the present invention provides the use of a compound of the invention for the preparation of a medicament for the treatment of a condition which directly or indirectly results from mutation of a Raf or ErbB family kinase or overexpression of a Raf or ErbB family kinase, or a mutation of an upstream activator of a Raf or ErbB family kinase or overexpression of an upstream activator of a Raf or ErbB family kinase in a mammal. Conditions which are mediated by at least one Raf family kinase and conditions which are mediated by at least one ErbB family kinase, and particularly conditions mediated by inappropriate activity of one or Raf or ErbB family kinases, including those which directly or indirectly result from mutation of a Raf or ErbB family kinase, overexpression of a Raf or ErbB family kinase, or mutation of an upstream activator of a Raf or ErbB family kinase or overexpression of an upstream activator of a Raf or ErbB family kinase are known in the art and include but are not limited to neoplasms.

Compounds of the invention may also be used in the treatment of conditions attenuated by inhibition of a Raf family kinase (particularly B-Raf) or inhibition of an ErbB family kinase (particularly ErbB2). Further provided are methods for treating a condition attenuated by inhibition of a Raf family kinase (particularly B-Raf) or inhibition of an ErbB family kinase (particularly ErbB2) in a mammal in need thereof, comprising administering to the mammal a therapeutically effective amount of a compound of the invention. Also provided is the use of a compound of the invention for the preparation of a medicament for the treatment of a condition attenuated by inhibition of a Raf family kinase (particularly B-Raf) or inhibition of an ErbB family kinase (particularly ErbB2) in a mammal. Conditions attenuated by inhibition of a Raf family kinase (including B-Raf) or inhibition of an ErbB family kinase (particularly ErbB2) include but are not limited to neoplasms.

Accordingly, compounds of the invention may be used in the treatment of a neoplasm, particularly a susceptible neoplasm (a cancer or tumor) in a mammal. The present invention also provides a method for treating a neoplasm, particularly a susceptible neoplasm in a mammal in need thereof, which method comprises administering to the mammal a therapeutically effective amount of the compound of the invention. The invention also provides the use of a compound of the invention for the preparation of a medicament for the treatment of neoplasm, particularly a susceptible neoplasm, in a mammal. “Susceptible neoplasm” as used herein refers to neoplasms which are susceptible to treatment by a kinase inhibitor and particularly neoplasms that are susceptible to treatment by either a Raf inhibitor or an ErbB inhibitor. Neoplasms which have been associated with inappropriate activity of one or more Raf family kinases and particularly neoplasms which are exhibit mutation of a Raf family kinase, overexpression of a Raf family kinase, or mutation of an upstream activator of a Raf family kinase or overexpression of an upstream activator of a Raf family kinase, and are therefore susceptible to treatment with an Raf inhibitor are known in the art, and include both primary and metastatic tumors and cancers. See, Catalogue of Somatic Mutations in Cancer (COSMIC), the Wellcome Trust Sanger Institute, http://www.sanger.ac.uk/genetics/CGP/cosmic/ and those references cited in the background. Neoplasms which have been associated with inappropriate activity of one or more ErbB family kinases and particularly neoplasms which are exhibit mutation of an ErbB family kinase, overexpression of an ErbB family kinase, or mutation of an upstream activator of an ErbB family kinase or overexpression of an upstream activator of an ErbB family kinase, and are therefore susceptible to treatment with an ErbB inhibitor are known in the art, and include both primary and metastatic tumors and cancers. See, references cited in the background.

Specific examples of susceptible neoplasms within the scope of the invention include, but are not limited to:

Barret's adenocarcinoma;
billiary tract carcinomas;
bladder cancer
breast cancer;
cervical cancer;
cholangiocarcinoma;
central nervous system tumors including primary CNS tumors such as glioblastomas, astrocytomas (including glioblastoma multiforme) and ependymomas, and secondary CNS tumors (i.e., metastases to the central nervous system of tumors originating outside of the central nervous system),
   colorectal cancer, including large intestinal colon carcinoma;
   esophageal cancer
   gastric cancer;
carcinoma of the head and neck including squamous cell carcinoma of the head and neck;
hematologic cancers including leukemias and lymphomas such as acute lymphoblastic leukemia, acute myelogenous leukemia (AML), myelodysplastic syndromes, chronic myelogenous leukemia, Hodgkin's lymphoma, non-Hodgkin's lymphoma, megakaryoblastic leukemia, multiple myeloma and erythroleukemia;
   hepatocellular carcinoma;
lung cancer including small cell lung cancer, non-small cell lung cancer and squamous cell lung cancer;
   ovarian cancer, endometrial cancer, cervical cancer;
   pancreatic cancer;
   pituitary adenoma;
   prostate cancer;
   renal cancer;
   sarcoma;
   skin cancers including melanomas;
   thyroid cancers; and
   uterine cancer.

Accordingly, in one embodiment, the present invention provides a method for the treatment of Barret's adenocarcinoma; billiary tract carcinomas; bladder cancer; breast cancer; cervical cancer; cholangiocarcinoma; central nervous system tumors including primary CNS tumors such as glioblastomas, astrocytomas (including glioblastoma multiforme) and ependymomas, and secondary CNS tumors (i.e., metastases to the central nervous system of tumors originating outside of the central nervous system); colorectal cancer, including large intestinal colon carcinoma; esophageal cancer; gastric cancer; carcinoma of the head and neck including squamous cell carcinoma of the head and neck; hematologic cancers including leukemias and lymphomas such as acute lymphoblastic leukemia, acute myelogenous leukemia (AML), myelodysplastic syndromes, chronic myelogenous leukemia, Hodgkin's lymphoma, non-Hodgkin's lymphoma, megakaryoblastic leukemia, multiple myeloma and erythroleukemia; hepatocellular carcinoma; lung cancer including small cell lung cancer, non-small cell lung cancer and squamous cell lung cancer; ovarian cancer; endometrial cancer; cervical cancer; pancreatic cancer; pituitary adenoma; prostate cancer; renal cancer; sarcoma; skin cancers including melanomas; thyroid cancers; and uterine cancer, or any subset thereof, in a mammal in need thereof, the method comprising administering a therapeutically effective amount of a compound of the invention to the mammal.

The present invention also provides the a compound of formula (I) for use in the treatment of Barret's adenocarcinoma; billiary tract carcinomas; bladder cancer; breast cancer; cervical cancer; cholangiocarcinoma; central nervous system tumors including primary CNS tumors such as glioblastomas, astrocytomas (including glioblastoma multiforme) and ependymomas, and secondary CNS tumors (i.e., metastases to the central nervous system of tumors originating outside of the central nervous system); colorectal cancer, including large intestinal colon carcinoma; esophageal cancer; gastric cancer; carcinoma of the head and neck including squamous cell carcinoma of the head and neck; hematologic cancers including leukemias and lymphomas such as acute lymphoblastic leukemia, acute myelogenous leukemia (AML), myelodysplastic syndromes, chronic myelogenous leukemia, Hodgkin's lymphoma, non-Hodgkin's lymphoma, megakaryoblastic leukemia, multiple myeloma and erythroleukemia; hepatocellular carcinoma; lung cancer including small cell lung cancer, non-small cell lung cancer and squamous cell lung cancer; ovarian cancer; endometrial cancer; cervical cancer; pancreatic cancer; pituitary adenoma; prostate cancer; renal cancer; sarcoma; skin cancers including melanomas; thyroid cancers; and uterine cancer, or any subset thereof, in a mammal in need thereof.

The present invention further provides the use of a compound of formula (I) for the preparation of a medicament for the treatment of Barret's adenocarcinoma; billiary tract carcinomas; bladder cancer; breast cancer; cervical cancer; cholangiocarcinoma; central nervous system tumors including primary CNS tumors such as glioblastomas, astrocytomas (including glioblastoma multiforme) and ependymomas, and secondary CNS tumors (i.e., metastases to the central nervous system of tumors originating outside of the central nervous system); colorectal cancer, including large intestinal colon carcinoma; esophageal cancer; gastric cancer; carcinoma of the head and neck including squamous cell carcinoma of the head and neck; hematologic cancers including leukemias and lymphomas such as acute lymphoblastic leukemia, acute myelogenous leukemia (AML), myelodysplastic syndromes, chronic myelogenous leukemia, Hodgkin's lymphoma, non-Hodgkin's lymphoma, megakaryoblastic leukemia, multiple myeloma and erythroleukemia; hepatocellular carcinoma; lung cancer including small cell lung cancer, non-small cell lung cancer and squamous cell lung cancer; ovarian cancer; endometrial cancer; cervical cancer; pancreatic cancer; pituitary adenoma; prostate cancer; renal cancer; sarcoma; skin cancers including melanomas; thyroid cancers; and uterine cancer, or any subset thereof, in a mammal in need thereof.

As is well known in the art, tumors may metastasize from a first or primary locus of tumor to one or more other body tissues or sites. In particular, metastases to the central nervous system (i.e., secondary CNS tumors), and particularly the brain (i.e., brain metastases), are well documented for tumors and cancers, such as breast, lung, melanoma, renal and colorectal. As used herein, reference to uses or methods for treatment or treatments for “tumor” or “cancer” in a subject includes use for and treatment of the primary neoplasm, tumor or cancer, and where appropriate, also the use for and treatment of metastases (i.e., metastatic tumor growth) as well.

In one particular embodiment, the invention provides a method for treating breast cancer in a mammal in need thereof, comprising administering to the mammal a therapeutically effective amount of a compound of the invention. In one particular embodiment, the invention provides a method for treating colorectal cancer in a mammal in need thereof, comprising administering to the mammal a therapeutically effective amount of a compound of the invention. In one particular embodiment, the invention provides a method for treating melanoma in a mammal in need thereof, comprising administering to the mammal a therapeutically effective amount of a compound of the invention. In one particular embodiment, the invention provides a method for treating non-small cell lung cancer in a mammal in need thereof, comprising administering to the mammal a therapeutically effective amount of a compound of the invention. In one particular embodiment, the invention provides a method for treating ovarian cancer in a mammal in need thereof, comprising administering to the mammal a therapeutically effective amount of a compound of the invention. In one particular embodiment, the invention provides a method for treating thyroid cancer in a mammal in need thereof, comprising administering to the mammal a therapeutically effective amount of a compound of the invention.

In one particular embodiment, the susceptible neoplasm is breast cancer and the invention provides compounds for use in the treatment of breast cancer in a mammal and the use of such compounds for the preparation of a medicament for the treatment of breast cancer in a mammal. In another embodiment, the susceptible neoplasm is colorectal cancer and the invention provides compounds for use in the treatment of colorectal cancer in a mammal and the use of such compounds for the preparation of a medicament for the treatment of colorectal cancer in a mammal. In another embodiment, the susceptible neoplasm is melanoma, and the invention provides compounds for use in the treatment of melanoma in a mammal and the use of such compounds for the preparation of a medicament for the treatment of melanoma in a mammal. In another embodiment, the susceptible neoplasm is non-small cell lung cancer, and the invention provides compounds for use in the treatment of non-small cell lung cancer in a mammal and the use of such compounds for the preparation of a medicament for the treatment of non-small cell lung cancer in a mammal. In another embodiment, the susceptible neoplasm is ovarian cancer and the invention provides compounds for use in the treatment of ovarian cancer in a mammal and the use of such compounds for the preparation of a medicament for the treatment of ovarian cancer in a mammal. In another embodiment, the susceptible neoplasm is thyroid cancer, and the invention provides compounds for use in the treatment of thyroid cancer in a mammal and the use of such compounds for the preparation of a medicament for the treatment of thyroid cancer in a mammal.

The compounds of the invention can be used alone in the treatment of each of the foregoing conditions or can be used to provide additive or potentially synergistic effects with certain existing chemotherapies, radiation, biological or immunotherapeutics (including monoclonal antibodies) and vaccines. The compounds of the invention may be useful for restoring effectiveness of certain existing chemotherapies and radiation and or increasing sensitivity to certain existing chemotherapies and/or radiation. As used herein, the term “therapeutically effective amount” means an amount of a compound of the invention which is sufficient, in the subject to which it is administered, to elicit the biological or medical response of a cell culture, tissue, system, mammal (including human) that is being sought, for instance, by a researcher or clinician. The term also includes within its scope amounts effective to enhance normal physiological function. For example, a therapeutically effective amount of a compound of the invention for the treatment of a condition mediated by at least one Raf family kinase or at least one ErbB family kinase is an amount sufficient to treat the condition in the subject. Similarly, a therapeutically effective amount of a compound of the invention for the treatment of a susceptible neoplasm is an amount sufficient to treat the particular susceptible neoplasm in the subject. In one embodiment of the present invention, a therapeutically effective amount of a compound of the invention is an amount sufficient to regulate, modulate, bind or inhibit at least one Raf family kinase or at least one ErbB family kinase. More particularly, in such embodiment, the therapeutically effective amount of a compound of the invention is an amount sufficient to regulate, modulate, bind or inhibit B-Raf and/or ErbB2 kinases.

The precise therapeutically effective amount of the compounds of the invention will depend on a number of factors. There are variables inherent to the compounds including, but not limited to, the following: molecular weight, inhibitory activity at the target kinase, absorption, bioavailability, distribution in the body, tissue penetration, half-life, metabolism, protein binding, and excretion. These variables determine what dose of compound needs to be administered in order to inhibit the target kinase by a sufficient percentage and for a sufficient amount of time to have the desired effect on the tumor. In general, the goal will be to inhibit the target kinase by 50% or more for as long as possible. The duration of drug exposure will be limited only by the compound half-life, and side effects from treatment requiring cessation of dosing. The amount of compound administered will also depend on factors related to patients and disease including, but not limited to, the following: the age, weight, concomitant medications and medical condition of the subject being treated, the precise condition requiring treatment and its severity, the nature of the formulation, and the route of administration. Ultimately the dose will be at the discretion of the attendant physician or veterinarian. Typically, the compound of the invention will be given for treatment in the range of 0.01 to 30 mg/kg body weight of recipient (mammal) per day and more usually in the range of 0.1 to 10 mg/kg body weight per day. Thus, for a 70 kg adult human being treated for a condition mediated by at least one Raf family kinase or at least one ErbB family kinase, the actual amount per day would usually be from 1 to 2000 mg and this amount may be given in a single or multiple doses per day. Dosing regimens may vary significantly and will be determined and altered based on clinical experience with the compound. The full spectrum of dosing regimens may be employed ranging from continuous dosing (with daily doses) to intermittent dosing. A therapeutically effective amount of a pharmaceutically acceptable salt of a compound of formula (I) may be determined as a proportion of the therapeutically effective amount of the compound of formula (I) as the free base. It is envisaged that similar dosages would be appropriate for treatment of the susceptible neoplasms described above.

While it is possible that, for use in therapy, a therapeutically effective amount of a compound of the invention may be administered as the raw chemical, it is typically presented as the active ingredient of a pharmaceutical composition or formulation. Accordingly, the invention further provides a pharmaceutical composition comprising a compound of the invention. The pharmaceutical composition may further comprise one or more pharmaceutically acceptable carriers, diluents, and/or excipients. The carrier(s), diluent(s) and/or excipient(s) must be acceptable in the sense of being compatible with the other ingredients of the formulation and not deleterious to the recipient thereof. In accordance with another aspect of the invention there is also provided a process for the preparation of a pharmaceutical formulation including admixing a compound of the invention with one or more pharmaceutically acceptable carriers, diluents and/or excipients.

Pharmaceutical formulations may be presented in unit dose forms containing a predetermined amount of active ingredient per unit dose. Such a unit may contain, for example, 0.5 mg to 1 g, preferably 1 mg to 700 mg, more preferably 5 mg to 100 mg of a compound of the invention (as a free-base, solvate (including hydrate) or salt, in any form), depending on the condition being treated, the route of administration, and the age, weight and condition of the patient. Preferred unit dosage formulations are those containing a daily dose, weekly dose, monthly dose, a sub-dose or an appropriate fraction thereof, of an active ingredient. Furthermore, such pharmaceutical formulations may be prepared by any of the methods well known in the pharmacy art.

Pharmaceutical formulations may be adapted for administration by any appropriate route, for example by the oral (including capsules, tablets, liquid-filled capsules, disintegrating tablets, immediate, delayed and controlled release tablets, oral strips, solutions, syrups, buccal and sublingual), rectal, nasal, inhalation, topical (including transdermal), vaginal or parenteral (including subcutaneous, intramuscular, intravenous or intradermal) route. Such formulations may be prepared by any method known in the art of pharmacy, for example by bringing into association the active ingredient with the carrier(s), excipient(s) or diluent. Generally, the carrier, excipient or diluent employed in the pharmaceutical formulation is “non-toxic,” meaning that it/they is/are deemed safe for consumption in the amount delivered in the pharmaceutical composition, and “inert” meaning that it/they does/do not appreciably react with or result in an undesired effect on the therapeutic activity of the active ingredient.

Pharmaceutical formulations adapted for oral administration may be presented as discrete units such as liquid-filled or solid capsules; immediate, delayed or controlled release tablets; powders or granules; solutions or suspensions in aqueous or non-aqueous liquids; edible foams or whips; oil-in-water liquid emulsions, water-in-oil liquid emulsions or oral strips, such as impregnated gel strips.

For instance, for oral administration in the form of a tablet or capsule, the active drug component can be combined with an oral pharmaceutically acceptable carrier such as ethanol, glycerol, water and the like. Powders are prepared by comminuting the compound to a suitable fine size and mixing with a similarly comminuted pharmaceutical carrier such as an edible carbohydrate, as, for example, starch or mannitol. Flavoring, preservative, dispersing and coloring agent can also be present.

Solid capsules are made by preparing a powder mixture, as described above, and filling formed gelatin sheaths. Glidants and lubricants such as colloidal silica, talc, magnesium stearate, calcium stearate or solid polyethylene glycol can be added to the powder mixture before the filling operation. A disintegrating or solubilizing agent such as agar-agar, calcium carbonate or sodium carbonate can also be added to improve the availability of the medicament when the capsule is ingested.

Moreover, when desired or necessary, suitable binders, lubricants, disintegrating agents and coloring agents can also be incorporated into the mixture. Suitable binders include starch, gelatin, natural sugars such as glucose or beta-lactose, corn sweeteners, natural and synthetic gums such as acacia, tragacanth or sodium alginate, carboxymethylcellulose, polyethylene glycol, waxes and the like. Lubricants used in these dosage forms include sodium oleate, sodium stearate, magnesium stearate, sodium benzoate, sodium acetate, sodium chloride and the like. Disintegrators include, without limitation, starch, methyl cellulose, agar, bentonite, xanthan gum and the like. Tablets are formulated, for example, by preparing a powder mixture, granulating or slugging, adding a lubricant and disintegrant and pressing into tablets. A powder mixture is prepared by mixing the compound, suitably comminuted, with a diluent or base as described above, and optionally, with a binder such as carboxymethylcellulose, an alginate, gelatin, or polyvinyl pyrrolidone, a solution retardant such as paraffin, a resorption accelerator such as a quaternary salt and/or an absorption agent such as bentonite, kaolin or dicalcium phosphate. The powder mixture can be granulated by wetting with a binder such as syrup, starch paste, acadia mucilage or solutions of cellulosic or polymeric materials and forcing through a screen. As an alternative to granulating, the powder mixture can be run through the tablet machine and the result is imperfectly formed slugs broken into granules. The granules can be lubricated to prevent sticking to the tablet forming dies by means of the addition of stearic acid, a stearate salt, talc or mineral oil. The lubricated mixture is then compressed into tablets. The compounds of the present invention can also be combined with a free flowing inert carrier and compressed into tablets directly without going through the granulating or slugging steps. A clear or opaque protective coating consisting of a sealing coat of shellac, a coating of sugar or polymeric material and a polish coating of wax can be provided. Dyestuffs can be added to these coatings to distinguish different unit dosages.

Oral fluids such as solutions, syrups and elixirs can be prepared in dosage unit form so that a given quantity contains a predetermined amount of the compound. Solutions and syrups can be prepared by dissolving the compound in a suitably flavored aqueous solution, while elixirs are prepared through the use of a pharmaceutically acceptable alcoholic vehicle. Suspensions can be formulated by dispersing the compound in a pharmaceutically acceptable vehicle. Solubilizers and emulsifiers such as ethoxylated isostearyl alcohols and polyoxy ethylene sorbitol ethers, preservatives, flavor additive such as peppermint oil or natural sweeteners or saccharin or other artificial sweeteners, and the like can also be added.

Where appropriate, unit dosage formulations for oral administration can be microencapsulated. The formulation can also be prepared to prolong or sustain the release as for example by coating or embedding particulate material in polymers, wax or the like.

The compounds of the invention can also be administered in the form of liposome delivery systems, such as small unilamellar vesicles, large unilamellar vesicles and multilamellar vesicles. Liposomes can be formed from a variety of phospholipids, such as cholesterol, stearylamine or phosphatidylcholines.

The compounds of the invention may also be delivered by the use of monoclonal antibodies as individual carriers to which the compound molecules are coupled. The compounds may also be coupled with soluble polymers as targetable drug carriers. Such polymers can include polyvinylpyrrolidone, pyran copolymer, polyhydroxypropyl-methacrylamidephenol, polyhydroxyethylaspartamidephenol, or polyethyleneoxide-polylysine substituted with palmitoyl residues. Furthermore, the compounds may be coupled to a class of biodegradable polymers useful in achieving controlled release of a drug, for example, polycentric acid, polepsilon caprolactone, polyhydroxy butyric acid, polyorthoesters, polyacetals, polydihydropyrans, polycyanoacrylates and cross-linked or amphipathic block copolymers of hydrogels.

Pharmaceutical formulations adapted for transdermal administration may be presented as discrete patches intended to remain in intimate contact with the epidermis of the recipient for a prolonged period of time. For example, the active ingredient may be delivered from the patch by iontophoresis as generally described in Pharmaceutical Research (1986) 3(6):318.

Pharmaceutical formulations adapted for topical administration may be formulated as ointments, creams, suspensions, lotions, powders, solutions, pastes, gels, sprays, aerosols or oils. For treatments of external tissues, such as skin, the formulations may be applied as a topical ointment or cream. When formulated in an ointment, the active ingredient may be employed with either a paraffinic or a water-miscible ointment base. Alternatively, the active ingredient may be formulated in a cream with an oil-in-water cream base or a water-in-oil base. Pharmaceutical formulations adapted for topical administrations to the eye include eye drops wherein the active ingredient is dissolved or suspended in a suitable carrier, especially an aqueous solvent. Pharmaceutical formulations adapted for topical administration in the mouth include lozenges, pastilles and mouth washes.

Pharmaceutical formulations adapted for rectal administration may be presented as suppositories or as enemas.

Pharmaceutical formulations adapted for nasal administration wherein the carrier is a solid include a coarse powder having a particle size for example in the range 20 to 500 microns which is administered in the manner in which snuff is taken, i.e. by rapid inhalation through the nasal passage from a container of the powder held close up to the nose. Suitable formulations wherein the carrier is a liquid, for administration as a nasal spray or as nasal drops, include aqueous or oil solutions of the active ingredient.

Pharmaceutical formulations adapted for administration by inhalation include fine particle dusts or mists, which may be generated by means of various types of metered dose pressurized aerosols, metered dose inhalers, dry powder inhalers, nebulizers or insufflators.

Pharmaceutical formulations adapted for vaginal administration may be presented as pessaries, tampons, creams, gels, pastes, foams or spray formulations.

Pharmaceutical formulations adapted for parenteral administration include aqueous and non-aqueous sterile injection solutions which may contain anti-oxidants, buffers, bacteriostats and solutes which render the formulation of pharmaceutically acceptable tonicity with the blood of the intended recipient; and aqueous and non-aqueous sterile suspensions which may include suspending agents and thickening agents. The formulations may be presented in unit-dose or multi-dose containers, for example sealed ampoules and vials, and may be stored in a freeze-dried (lyophilized) condition requiring only the addition of the sterile liquid carrier, for example water for injection, immediately prior to use. Extemporaneous injection solutions and suspensions may be prepared from sterile powders, granules and tablets.

It should be understood that in addition to the ingredients particularly mentioned above, the formulations may include other agents conventional in the art having regard to the type of formulation in question, for example those suitable for oral administration may include flavoring agents.

In the above-described methods of treatment and uses, a compound of the invention may be employed alone, in combination with one or more other compounds of the invention or in combination with other therapeutic methods or agents. In particular, in methods of treating a condition improved by inhibition of at least one Raf family kinase and/or at least one ErbB family kinase and in methods of treating susceptible neoplasms, combination with other chemotherapeutic, biologic, hormonal, antibody and supportive care agents is envisaged as well as combination with surgical therapy and radiotherapy. Supportive care agents include analgesics, anti-emetics and agents used to treat heamatologic side effects such as neutropenia. Analgesics are well known in the art. Anti-emetics include but are not limited to 5HT₃ antagonists such as ondansetron, granisetron, dolasetron, palonosetron and the like; prochlorperazine, metaclopromide, diphenhydramine, promethazine; dexamethasone, lorazepam; haloperidol, dronabinol, olanzapine; and neurokinin-1 antagonists such as aprepitant, fosaprepitant and casopitant administered alone or in various combinations.

The term “chemotherapeutic” as used herein refers to any chemical agent having a therapeutic effect on the subject to which it is administered. “Chemotherapeutic” agents include but are not limited to anti-neoplastic agents. As used herein, “anti-neoplastic agents” include both cytotoxic and cytostatic agents including biological, immunological and vaccine therapies. Combination therapies according to the invention thus comprise the administration of at least one compound of the invention and the use of at least one other treatment method. In one embodiment, combination therapies according to the invention comprise the administration of at least one compound of the invention and surgical therapy. In one embodiment, combination therapies according to the invention comprise the administration of at least one compound of the invention and radiotherapy. In one embodiment, combination therapies according to the invention comprise the administration of at least one compound of the invention and at least one supportive care agent (e.g., at least one anti-emetic agent). In one embodiment, combination therapies according to the present invention comprise the administration of at least one compound of the invention and at least one other chemotherapeutic agent. In one particular embodiment, the invention comprises the administration of at least one compound of the invention and at least one anti-neoplastic agent.

As an additional aspect, the present invention provides the methods of treatment and uses as described above, which comprise administering a compound of the invention together with at least one chemotherapeutic agent. In one particular embodiment, the chemotherapeutic agent is an anti-neoplastic agent. In another embodiment, the invention provides a pharmaceutical composition as described above further comprising at least one other chemotherapeutic agent, more particularly, the chemotherapeutic agent is an anti-neoplastic agent. The invention also provides methods of treatment and uses as described above, which comprise administering a compound of the invention together with at least one supportive care agent (e.g., anti-emetic agent).

The compounds of the invention and at least one additional anti-neoplastic or supportive care therapy may be employed in combination concomitantly or sequentially in any therapeutically appropriate combination. The administration of a compound of the invention with one or more other anti-neoplastic agents may be in combination in accordance with the invention by administration concomitantly in (1) a unitary pharmaceutical composition including both or all compounds or (2) separate pharmaceutical compositions each including one or more of the compounds. The components of the combination may be administered separately in a sequential manner wherein one active ingredient is administered first and the other(s) second or vice versa. Such sequential administration may be close in time or remote in time. When a compound of the invention is used in combination with an anti-neoplastic and/or supportive care agent, the dose of each compound may differ from that when the compound is used alone. Appropriate doses will be readily appreciated by those skilled in the art. The appropriate dose of the compound(s) of the invention and the other therapeutically active agent(s) and the relative timings of administration will be selected in order to achieve the desired combined therapeutic effect, and are within the expertise and discretion of the attendant clinician.

Typically, any chemotherapeutic agent that has activity against a susceptible neoplasm being treated may be utilized in combination with the compounds the invention, provided that the particular agent is clinically compatible with therapy employing a compound of the invention. Typical anti-neoplastic agents useful in the present invention include, but are not limited to: alkylating agents, anti-metabolites, antitumor antibiotics, antimitotic agents, topoisomerase I and II inhibitors, hormones and hormonal analogues; signal transduction pathway inhibitors including inhibitors of cell growth or growth factor function, angiogenesis inhibitors, and serine/threonine or other kinase inhibitors; cyclin dependent kinase inhibitors; antisense therapies and immunotherapeutic agents, including monoclonals, vaccines or other biological agents.

Alkylating agents are non-phase specific anti-neoplastic agents and strong electrophiles. Typically, alkylating agents form covalent linkages, by alkylation, to DNA through nucleophilic moieties of the DNA molecule such as phosphate, amino, and hydroxyl groups. Such alkylation disrupts nucleic acid function leading to cell death. Alkylating agents may be employed in combination with the compounds of the invention in the compositions and methods described above. Examples of alkylating agents include but are not limited to nitrogen mustards such as cyclophosphamides, temozolamide, melphalan, and chlorambucil; oxazaphosphorines; alkyl sulfonates such as busulfan; nitrosoureas such as carmustine; triazenes such as dacarbazine; and platinum coordination complexes such as cisplatin, oxaliplatin and carboplatin.

Antimetabolite neoplastic agents are phase specific anti-neoplastic agents that act at S phase (DNA synthesis) of the cell cycle by inhibiting DNA synthesis or by inhibiting purine or pyrimidine base synthesis and thereby limiting DNA synthesis. The end result of discontinuing S phase is cell death. Antimetabolite neoplastic agents may be employed in combination with the compounds of the invention in the compositions and methods described above. Examples of antimetabolite anti-neoplastic agents include, but are not limited to, purine and pyrimidine analogues and anti-folate compounds, and more specifically, hydroxyurea, cytosine, arabinoside, ralitrexed, tegafur, fluorouracil (e.g., 5FU), methotrexate, cytarabine, mercaptopurine and thioguanine.

Antitumor antibiotic agents are non-phase specific agents, which bind to or intercalate with DNA. Typically, such action disrupts ordinary function of the nucleic acids, leading to cell death. Antitumor antibiotics may be employed in combination with the compounds of the invention in the compositions and methods described above. Examples of antitumor antibiotic agents include, but are not limited to, actinomycins such as dactinomycin; anthracyclines such as daunorubicin, doxorubicin, idarubicin, epirubicin and mitoxantrone; mitomycin C and bleomycins.

Antimicrotubule or antimitotic agents are phase specific agents active against the microtubules of tumor cells during M or the mitosis phase of the cell cycle. Antimitotic agents may be employed in combination with the compounds of the invention in the compositions and methods described above. Examples of antimitotic agents include, but are not limited to, diterpenoids, vinca alkaloids, polo-like kinase (Plk) inhibitors and CenpE inhibitors. Examples of diterpenoids include, but are not limited to, paclitaxel and its analog docetaxel. Examples of vinca alkaloids include, but are not limited to, vinblastine, vincristine, vindesine and vinorelbine. Plk inhibitors are discussed further below.

Topoisomerase inhibitors include inhibitors of Topoisomerase II and inhibitors of Topoisomerase I. Topoisomerase II inhibitors, such as epipodophyllotoxins, are anti-neoplastic agents derived from the mandrake plant, that typically affect cells in the S and G₂ phases of the cell cycle by forming a ternary complex with topoisomerase II and DNA, causing DNA strand breaks. The strand breaks accumulate and cell death follows. Examples of epipodophyllotoxins include, but are not limited to, etoposide and teniposide. Camptothecins, including camptothecin and camptothecin derivatives, are available or under development as Topoisomerase I inhibitors. Examples of camptothecins include, but are not limited to amsacrine, irinotecan, topotecan, and the various optical forms of 7-(4-methylpiperazino-methylene)-10,11-ethylenedioxy-20-camptothecin. Topoisomerase inhibitors may be employed in combination with the compounds of the invention in the compositions and methods described above.

Hormones and hormonal analogues are useful compounds for treating cancers in which there is a relationship between the hormone(s) and growth and/or lack of growth of the cancer. Antitumor hormones and hormonal analogues may be employed in combination with the compounds of the invention in the compositions and methods described above. Examples of hormones and hormonal analogues believed to be useful in the treatment of neoplasms include, but are not limited to antiestrogens, such as tamoxifen, toremifene, raloxifene, fulvestrant, iodoxyfene and droloxifene; anti-androgens; such as flutamide, nilutamide, bicalutamide and cyproterone acetate; adrenocorticosteroids such as prednisone and prednisolone; aminoglutethimide and other aromatase inhibitors such as anastrozole, letrazole, vorazole, and exemestane; progestrins such as megestrol acetate; 5α-reductase inhibitors such as finasteride and dutasteride; and gonadotropin-releasing hormones (GnRH) and analogues thereof, such as Leutinizing Hormone-releasing Hormone (LHRH) agonists and antagonists such as goserelin luprolide, leuprorelin and buserelin.

Signal transduction pathway inhibitors are those inhibitors which block or inhibit a chemical process which evokes an intracellular change. As used herein this change is cell proliferation or differentiation or survival. Signal transduction pathway inhibitors useful in the present invention include, but are not limited to, inhibitors of receptor tyrosine kinases, non-receptor tyrosine kinases, SH2/SH3 domain blockers, serine/threonine kinases, phosphatidyl inositol-3-OH kinases, myoinositol signaling, and Ras oncogenes. Signal transduction pathway inhibitors may be employed in combination with the compounds of the invention in the compositions and methods described above.

Several protein tyrosine kinases catalyze the phosphorylation of specific tyrosine residues in various proteins involved in the regulation of cell growth. Such protein tyrosine kinases can be broadly classified as receptor or non-receptor kinases.

Receptor tyrosine kinase inhibitors which may be combined with the compounds of the invention include those involved in the regulation of cell growth, which receptor tyrosine kinases are sometimes referred to as “growth factor receptors.” Examples of growth factor receptor inhibitors, include but are not limited to inhibitors of: insulin growth factor receptors (IGF-1R, IR and IRR); epidermal growth factor family receptors (EGFR, ErbB2, and ErbB4); platelet-derived growth factor receptors (PDGFRs), vascular endothelial growth factor receptors (VEGFRs), tyrosine kinase with immunoglobulin-like and epidermal growth factor homology domains (TIE-2), macrophage colony stimulating factor (c-fms), c-kit, c-met, fibroblast growth factor receptors (FGFRs), hepatocyte growth factor receptors (HGFRs), Trk receptors (TrkA, TrkB, and TrkC), ephrin (Eph) receptors and the RET protooncogene.

Several inhibitors of growth factor receptors are under development and include ligand antagonists, antibodies, tyrosine kinase inhibitors, anti-sense oligonucleotides and aptamers. Any of these growth factor receptor inhibitors may be employed in combination with the compounds of the invention in any of the compositions and methods/uses described herein. Trastuzumab (Herceptin®) is an example of an anti-ErbB2 antibody inhibitor of growth factor function. One example of an anti-ErbB1 antibody inhibitor of growth factor function is cetuximab (Erbitux™, C225). Bevacizumab (Avastin®) is an example of a monoclonal antibody directed against VEGFR. Examples of small molecule inhibitors of epidermal growth factor receptors include but are not limited to lapatinib (Tykerb™) and erlotinib (TARCEVA®). Imatinib (GLEEVEC®) is one example of a PDGFR inhibitor. Examples of VEGFR inhibitors include pazopanib, ZD6474, AZD2171, PTK787, sunitinib and sorafenib.

In one embodiment, the invention provides methods of treatment of any of the various conditions enumerated above comprising administering a compound of the invention in combination with an EGFR or ErbB inhibitor. In one particular embodiment, the methods of the present invention comprise administering a compound of the invention in combination with lapatinib. In one particular embodiment, the methods of the present invention comprise administering a compound of the invention in combination with trastuzumab. In one particular embodiment, the methods of the present invention comprise administering a compound of the invention in combination with erlotinib. In one particular embodiment, the methods of the present invention comprise administering a compound of the invention in combination with gefitinib.

In another embodiment, the present invention provides methods of treatment of any of the various conditions enumerated above comprising administering a compound of the invention in combination with a VEGFR inhibitor. In one particular embodiment, the methods of the present invention comprise administering a compound of the invention in combination with pazopanib.

Tyrosine kinases that are not transmembrane growth factor receptor kinases are termed non-receptor, or intracellular tyrosine kinases. Inhibitors of non-receptor tyrosine kinases are sometimes referred to as “anti-metastatic agents” and are useful in the present invention. Targets or potential targets of anti-metastatic agents, include, but are not limited to, c-Src, Lck, Fyn, Yes, Jak, Abl kinase (c-Abl and Bcr-Abl), FAK (focal adhesion kinase) and Bruton's tyrosine kinase (BTK). Non-receptor kinases and agents, which inhibit non-receptor tyrosine kinase function, are described in Sinha, S, and Corey, S. J., (1999) J. Hematother. Stem Cell Res. 8:465-80; and Bolen, J. B. and Brugge, J. S., (1997) Annu. Rev. of Immunol. 15:371-404.

SH2/SH3 domain blockers are agents that disrupt SH2 or SH3 domain binding in a variety of enzymes or adaptor proteins including, but not limited to, Pl3-K p85 subunit, Src family kinases, adaptor molecules (Shc, Crk, Nck, Grb2) and Ras-GAP. Examples of Src inhibitors include, but are not limited to, dasatinib and BMS-354825 (J. Med. Chem. (2004) 47:6658-6661).

Inhibitors of serine/threonine kinases may also be used in combination with the compounds of the invention in any of the compositions and methods described above.

Examples of serine/threonine kinase inhibitors that may also be used in combination with a compound of the present invention include, but are not limited to, polo-like kinase inhibitors (Plk family e.g., Plk1, Plk2, and Plk3), which play critical roles in regulating processes in the cell cycle including the entry into and the exit from mitosis; MAP kinase cascade blockers, which include other Ras/Raf kinase inhibitors, mitogen or extracellular regulated kinases (MEKs), and extracellular regulated kinases (ERKs); Aurora kinase inhibitors (including inhibitors of Aurora A and Aurora B); protein kinase C (PKC) family member blockers, including inhibitors of PKC subtypes (alpha, beta, gamma, epsilon, mu, lambda, iota, zeta); inhibitors of kappa-B (IkB) kinase family (IKK-alpha, IKK-beta); PKB/Akt kinase family inhibitors; and inhibitors of TGF-beta receptor kinases. Examples of Plk inhibitors are described in PCT Publication No. WO04/014899 and WO07/03036 both to GlaxoSmithKline. Other examples of serine/threonine kinase inhibitors are known in the art. In another embodiment, the present invention provides methods of treatment of any of the various conditions enumerated above comprising administering a compound of the invention in combination with a Plk inhibitor. In one particular embodiment, the methods of the present invention comprise administering a compound of the invention in combination with 5-{6-[(4-Methylpiperazin-1-yl)methyl]-1H-benzimidazol-1-yl}-3-{(1R)-1-[2-(trifluoromethyl)phenyl]ethoxy}thiophene-2-carboxamide.

Urokinase, also referred to as urokinase-type Plasminogen Activator (uPA), is a serine protease. Activation of the serine protease plasmin triggers a proteolysis cascade which is involved in thrombolysis or extracellular matrix degradation. Elevated expression of urokinase and several other components of the plasminogen activation system have been correlated with tumor malignancy including several aspects of cancer biology such as cell adhesion, migration and cellular mitotic pathways as well. Inhibitors of urokinase expression may be used in combination with the compounds of the invention in the compositions and methods described above.

Inhibitors of Ras oncogene may also be useful in combination with the compounds of the present invention. Such inhibitors include, but are not limited to, inhibitors of farnesyltransferase, geranyl-geranyl transferase, and CAAX proteases as well as anti-sense oligonucleotides, ribozymes and immunotherapy. Such inhibitors have been shown to block Ras activation in cells containing mutant Ras, thereby acting as antiproliferative agents.

Inhibitors of kinases involved in the IGF-1R signaling axis may also be useful in combination with the compounds of the present invention. Such inhibitors include but are not limited to, inhibitors of JNK1/2/3, PI3K, AKT and MEK, and 14.3.3 signaling inhibitors. Examples of AKT inhibitors are described in PCT Publication No. WO 2007/058850, published 24 May 2007 which corresponds to PCT Application No. PCT/US2006/043513, filed 9 Nov. 2006, to GlaxoSmithKline. One particular AKT inhibitor disclosed therein is 4-(2-(4-amino-1,2,5-oxadiazol-3-yl)-1-ethyl-7-{[(3S)-3-piperidinylmethyl]oxy}-1H-imidazo[4,5-c]pyridin-4-yl)-2-methyl-3-butyn-2-ol.

Cell cycle signaling inhibitors, including inhibitors of cyclin dependent kinases (CDKs) are also useful in combination with the compounds of the invention in the compositions and methods described above. Examples of cyclin dependent kinases, including CDK2, CDK4, and CDK6 and inhibitors for the same are described in, for instance, Rosania G. R., et al., Exp. Opin. Ther. Patents (2000) 10:215-230.

Receptor kinase angiogenesis inhibitors may also find use in the present invention. Inhibitors of angiogenesis related to VEGFR and TIE-2 are discussed above in regard to signal transduction inhibitors (both are receptor tyrosine kinases). Other inhibitors may be used in combination with the compounds of the invention. For example, anti-VEGF antibodies, which do not recognize VEGFR (the receptor tyrosine kinase), but bind to the ligand; small molecule inhibitors of integrin (alphaᵥ beta₃) that inhibit angiogenesis; endostatin and angiostatin (non-RTK) may also prove useful in combination with the compounds of the invention. One example of a VEGFR antibody is bevacizumab (AVASTIN®).

Inhibitors of phosphatidyl inositol-3-OH kinase family members including blockers of PI3-kinase, ATM, DNA-PK, and Ku may also be useful in combination with the present invention.

Also of potential use in combination with the compounds of the invention are myo-inositol signaling inhibitors such as phospholipase C blockers and myoinositol analogues.

Antisense therapies may also be used in combination with the compounds of the invention. Examples of such antisense therapies include those directed towards the targets described above such as ISIS 2503 and gene therapy approaches such as those using thymidine kinase or cytosine deaminase.

Agents used in immunotherapeutic regimens may also be useful in combination with the compounds of the invention. Immunotherapeutic regimens include ex-vivo and in-vivo approaches to increasing immunogenicity of patient tumor cells such as transfection with cytokines (IL-2, IL-4, GMCFS and MCFS), approaches to increase T-cell activity, approaches with transfected immune cells and approaches with anti-idiotypic antibodies. Another potentially useful immunotherapeutic regimen is monoclonal antibodies with wild-type Fc receptors that may illicit an immune response in the host (e.g., IGF-1R monoclonal antibodies).

Agents used in proapoptotic regimens (e.g., Bcl-2 antisense oligonucleotides) may also be used in combination with the compounds of the invention. Members of the Bcl-2 family of proteins block apoptosis. Upregulation of Bcl-2 has therefore been linked to chemoresistance. Studies have shown that the epidermal growth factor (EGF) stimulates anti-apoptotic members of the Bcl-2 family (i.e., mcl-1). Therefore, strategies designed to downregulate the expression of Bcl-2 in tumors have demonstrated clinical benefit and are now in Phase II/III trials, namely Genta's G3139 bcl-2 antisense oligonucleotide. Such proapoptotic strategies using the antisense oligonucleotide strategy for Bcl-2 are discussed in Water, J. S., et al., J. Clin. Oncol. (2000) 18:1812-1823; and Kitada, S., et al., Antisense Res. Dev. (1994) 4:71-79.

Compounds of formula (I) may be prepared using the processes described below. In all of the schemes described below, it is understood that protecting groups may be employed where necessary in accordance with general principles known to those of skill in the art, for example, see Green, T. W. and Wuts, P. G. M. (1991) Protecting Groups in Organic Synthesis, John Wiley & Sons. The selection of a particular protecting group and processes for installation and removal of protecting groups is within the skill of those in the art. The selection of processes for installation and removal of protecting groups as well as the reaction conditions and order of their execution shall be consistent with the preparation of compounds of formula (I).

Compounds of formula (I) wherein Y is moiety ii or moiety iii wherein Q² is —N(H)—, may be conveniently prepared by the methods outlined in Scheme 1 below.

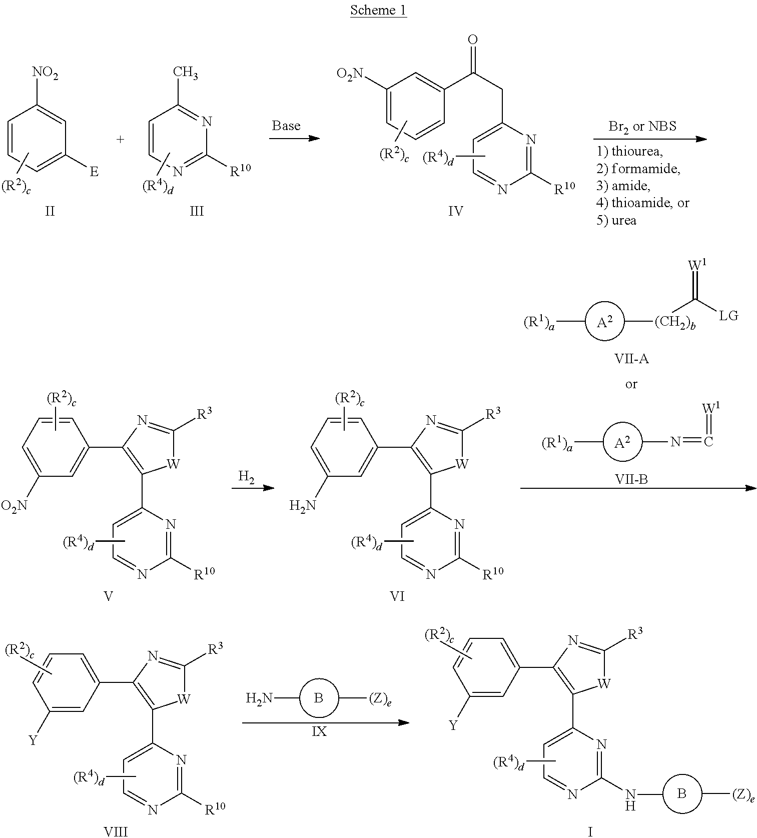

wherein:
R¹⁰ is halo (preferably chloro) or thiomethyl;
E is a suitable carboxylic ester or ester equivalent, particularly a methyl ester, ethyl ester, or Weinreb's amide;
   LG is a suitable leaving group;
   Ring A² is phenyl (moiety iii) or Ring A¹ (moiety ii);
   Y is moiety ii or moiety iii wherein Q² is —N(H)—; and
   all other variables are as defined above.

In this and subsequent synthetic routes, NBS is N-bromosuccinamide.

Generally, the process for preparing the compounds of formula (I) (all formulas and all variables having been defined above) comprises the step of:

reacting a compound of formula (VIII) with an aniline of formula (IX) to prepare a compound of formula (I).

More specifically, the process for preparing compounds of formula (I) wherein Y is moiety ii or moiety iii wherein Q² is —N(H)—, comprises the steps of:
a) condensing the compound of formula (II) with a substituted pyrimidine compound of formula (III) to prepare a compound of formula (IV);
b) reacting the compound of formula (IV) with a suitable brominating agent followed by one of:
   i) a thiourea,
   ii) a formamide,
   iii) an amide,
   iv) a thioamide, or
   v) a urea;
   to prepare a compound of formula (V);
c) reducing the compound of formula (V) to prepare a compound of formula (VI);
d) reacting a compound of formula (VI) with a compound of formula (VII-A) or a compound of formula (VII-B) to prepare a compound of formula (VIII);
e) reacting the compound of formula (VIII) with an aniline of formula (IX) to prepare a compound of formula (I);
f) optionally converting the compound of formula (I) to a pharmaceutically acceptable salt thereof; and
g) optionally converting the compound of formula (I) or a pharmaceutically acceptable salt thereof to a different compound of formula (I) or a pharmaceutically acceptable salt thereof.

As will be apparent to those skilled in the art, the order of the foregoing steps is not critical to the process of the present invention, and the process may be carried out using any suitable order of steps.

Compounds of formula (I) are prepared by reacting a compound of formula (VIII) with an aniline of formula (IX).

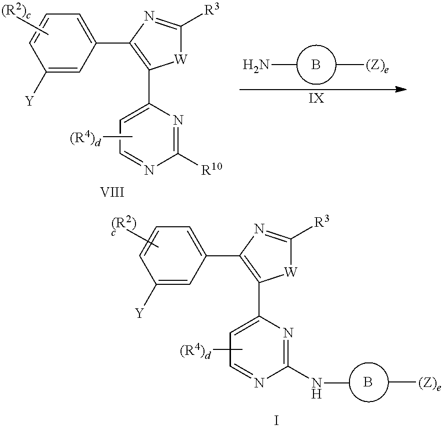

wherein all variables are as defined above.

Those skilled in the art will recognize that the conditions required for the above reaction will differ depending upon the definition of R¹⁰. When R¹⁰ is halo (preferably chloro), the reaction is generally performed in a solvent. Suitable solvents include but are not limited to isopropanol, 1,4-dioxane, ethanol, dimethylacetamide, trifluoroethanol, and N,N-dimethylformamide. The reaction is typically carried out under reflux conditions or in a microwave apparatus at a temperature of from about 90 to about 220° C., preferably from about 160 to about 190° C. As will be apparent to those skilled in the art of organic chemistry, it may be desirable to catalyze this reaction for the preparation of certain compounds of formula (I). For example, for compounds of formula (IX) and (I), wherein e is 0 and Ring B is aryl, it may be desirable to carry out the reaction in the presence of a catalytic amount of an acid such as hydrochloric acid, hydrobromic acid or para toluenesulfonic acid. As will further be apparent to those skilled in the art, it may also be desirable to install appropriate protecting groups prior to reacting the compound of formula (VIII) with the compound of formula (IX). For example, in the embodiment, wherein Z is a group containing a primary or secondary amine, the addition is preferably carried out when the amine is protected as, for example, its corresponding trifluoracetamide. The choice, installation and removal of appropriate protecting groups for reactions such as this, is conventional in the art. Compounds of formula (IX) are commercially available or may be synthesized using techniques conventional in the art.

When R¹⁰ is thiomethyl, the thiomethyl may first be converted to a more suitable leaving group, for example sulfoxide, sulfone, or chloride. The thiomethyl can be converted into a sulfoxide or sulfone by oxidation with an appropriate oxidizing agent, for example oxone, sodium periodate, or meta-chloroperbenzoic acid, in an appropriate solvent, for example dichloromethane, methanol, or water. Those skilled in the art will recognize that this will produce an analogue of the compound of formula (VIII) in which R¹⁰ is a sulfoxide or sulfone. The oxidized product can then be reacted with an aniline of formula (IX) to generate a compound of formula (I).

These reactions are generally performed in a suitable solvent, for example 2-propanol, dimethylacetamide, or dioxane, optionally with the addition of acid, for example hydrochloric acid, and at a temperature of 25-110° C., preferably 70-90° C., or in a microwave reactor at a temperature of 90-220° C., preferably 160-190° C.

Alternately, the pyrimidinyl sulfoxide or sulfone can be converted to the corresponding hydroxyl pyrimidine by reaction with an appropriate aqueous acid, for example hydrochloric acid or acetic acid, at a temperature of 25-110° C., preferably 70-90° C. The hydroxyl pyrimidine can then be converted to a chloride using an appropriate chlorinating reagent, for example phosphorous oxychloride or thionyl chloride, optionally in a solvent, for example dichloromethane, at a temperature of 25-120° C., preferably 60-80° C. Those skilled in the art will recognize that this process will produce a compound of formula (VIII) wherein R¹⁰ is chloro, which can be reacted with an aniline of formula (IX) as described above.

Compounds of formula (VIII) may be prepared by reacting a compound of formula (VI) with either a compound of formula (VII-A) or (VII-B).

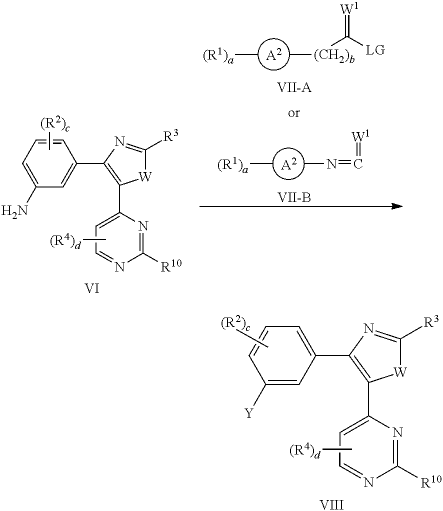

wherein all variables are as defined above in Scheme 1.

Reaction of the compound of formula (VI) with a compound of formula (VII-A) is suitable for the preparation of compounds of formula (I) wherein Y is moiety ii. Suitable leaving groups for the compounds of formula (VII-A) will be apparent to those skilled in the art and include, for example, halide and hydroxyl groups. Typically, the reaction is carried out in a suitable solvent such as dichloromethane or tetrahydrofuran, optionally with the addition of a suitable base, for example triethyl amine, at a temperature of −10° C. to 60° C., particularly 0° C. to 25° C. It will be understood by those skilled in the art that when LG is hydroxyl, the reaction may be carried out in the presence of an appropriate coupling agent such as dicyclohexylcarbodiimide (DCC) or ethylcarbodiimide hydrochloride (EDC). Compounds of formula (VII-A) are commercially available or may be synthesized using techniques conventional in the art. It will be appreciated by those skilled in the art that amide compounds of formula (VIII) may be converted to the corresponding thioamide compound of formula (VIII) using the conventional techniques.

Compounds of formula (VIII) wherein Y is a urea or thiourea linked phenyl according to moiety iii, may be prepared by reacting a compound of formula (VI) with a compound of formula (VII-B). This reaction may be carried out using conditions conventional in the art for such coupling reactions, including the use of a solvent such as tetrahydrofuran, 1,4-dioxane or dichloromethane at ambient temperature or with heating from about 40° C. to about 100° C. Compounds of formula (VII-B) are commercially available or may be synthesized using techniques conventional in the art.

Compounds of formula (VI) may be prepared by reducing a compound of formula (V).

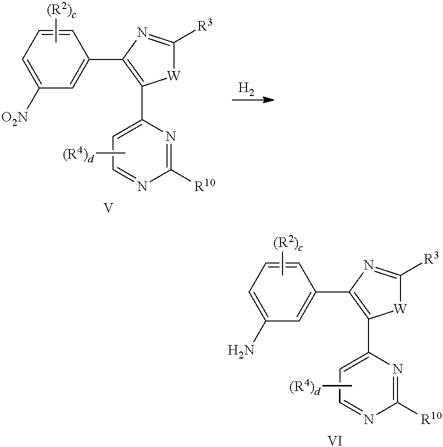

wherein all variables are as defined above in Scheme 1.

Those skilled in the art will recognize that this reaction may be accomplished in several ways. One process for the reduction of a compound of formula (V) involves the treatment of a compound of formula (V) with an atmosphere of hydrogen gas at about 14-100 psi, preferably about 30-50 psi in a suitable solvent, such as ethanol or methanol, and in the presence of a suitable catalyst, for example, platinum on carbon, palladium on carbon, or sulfided platinum on carbon. Another process for reducing a compound of formula (V) involves treating a compound of formula (V) with a suitable reducing agent such as sodium sulfide or tin tetrachloride, in a suitable solvent, for example, ethanol or tetrahydrofuran, optionally with the addition of a suitable acid, for example hydrochloric acid at a temperature of 25-100° C., particularly 50-70° C. Those skilled in the art will recognize that some reduction conditions include reagents, for example ethanol, that may also react with R¹⁰, for example when R¹⁰ is chloro. Those skilled in the art will also recognize that other reduction conditions described above or known to those of skill can be selected in such instances.

Compounds of formula (V) may be prepared by reacting a compound of formula (IV) with a suitable brominating reagent, particularly bromine or NBS, followed by reacting with one of: 1) a thiourea, 2) a formamide 3) an amide 4) a thioamide or 5) a urea depending upon whether the thiazole or oxazole and which particular substituent R³, is desired.

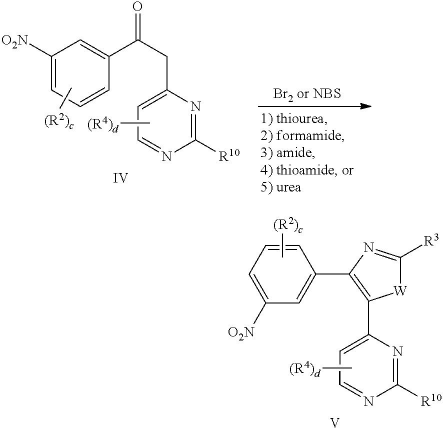

wherein all variables are as defined above.

In this and subsequent Schemes, reference to thiourea, formamide, amide, thioamide or urea in connection with this type of reaction refers to unsubstituted thiourea, formamide, amide, thioamide or urea and substituted analogs thereof. In particular, the thiourea, formamide, amide, thioamide or urea may be substituted with the desired group R³. Suitably substituted analogs of thiourea, formamide, amide, thioamide or urea are commercially available or may be prepared using conventional techniques.

When an aminothiazole (i.e., the compound of formula (V) wherein W is S and R³ is selected from —NR⁶R⁷, —N(R⁶)-cycloalkyl, —N(R⁶)Ph, —N(R⁶)Het, —N(R⁶)R⁵—Het, —N(R⁶)—R⁵—OR⁷, —N(R⁶)—R⁵—NR⁶R⁷, —N(H)C(O)R⁶, —N(R⁶)—C(O)—NR⁶R⁷, —N(H)SO₂R⁶, —N(R⁶)—R⁵—S(O)_{f}R⁷, and —N(R⁶)—S(O)₂—NR⁶R⁷, is desired, the reaction can be accomplished by the initial bromination of a compound of formula (IV) using an appropriate brominating reagent, for example bromine in solvent such as acetic acid or NBS.

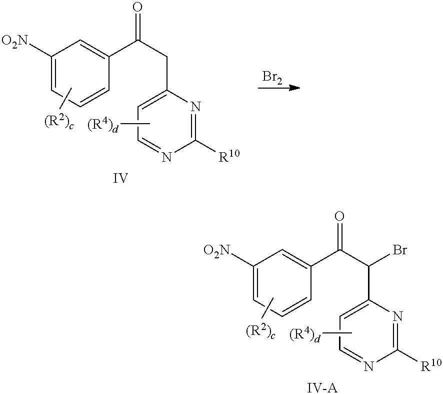

The reaction is typically carried out in an appropriate solvent, for example dichloromethane, N,N-dimethylformamide, or N,N-dimethylacetamide, and at a temperature of 25-50° C., particularly 25° C. The brominated analog (i.e., a compound of formula (IV-A)) is then reacted with an appropriately substituted thiourea.

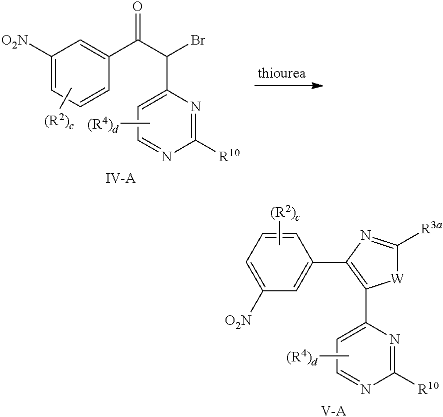

wherein R^{3a} is selected from —NR⁶R⁷, —N(R⁶)-cycloalkyl, —N(R⁶)Ph, —N(R⁶)Het, —N(R⁶)R⁵—Het, —N(R⁶)—R⁶—OR⁷, —N(R⁶)—R⁵—NR⁶R⁷, —N(H)C(O)R⁶, —N(R⁶)—C(O)—NR⁶R⁷, —N(H)SO₂R⁶, —N(R⁶)—R⁵—S(O)_{f}R⁷, and —N(R⁶)—S(O)₂—NR⁶R⁷;
   and all other variables are as defined above.

The reaction is typically carried out in an appropriate solvent, for example, dichloromethane, THF, dioxane, or acetonitrile, optionally in the presence of a suitable base, for example magnesium carbonate or sodium bicarbonate, and at a temperature of 25-90° C., particularly 25-50° C. Those skilled in the art will recognize that the thiourea can be unsubstituted, thus resulting in a compound of formula (V-A) wherein R³ is NH₂; or the thiourea may bear one or more additional substituents on one of the nitrogen atoms, for example as in N-[2-(4-morpholinyl)ethyl]thiourea.

In this and subsequent reactions, a compound, such as a compound of formula (V), wherein R³ is an amino group (or substituted amino), may be further converted to a corresponding compound wherein R³ is other than amino (or substituted amino) using the techniques described herein and those conventional in the art.

For example, the aminothiazole compound of formula (V-A) prepared according to the preceding description may be converted to an unsubstituted thiazole (i.e., a compound of formula (V) wherein R³ is H) using methods familiar to those of skill in the art. For example, the thiazole may be prepared by reacting the aminothiazole with an appropriate reagent, for example t-butyl nitrite, in an appropriate solvent, for example THF, and at a temperature of 35-75° C., particularly 40-60° C.

When a substituted thiazole is desired, an aminothiazole of formula (V-A) may be modified according to methods that will be familiar to those skilled in the art. For example, the aminothiazole compound of formula (V-A) may be converted to a compound of formula (V-B) by reaction with reagents capable of replacing the amino group with a halide, preferably a bromide.

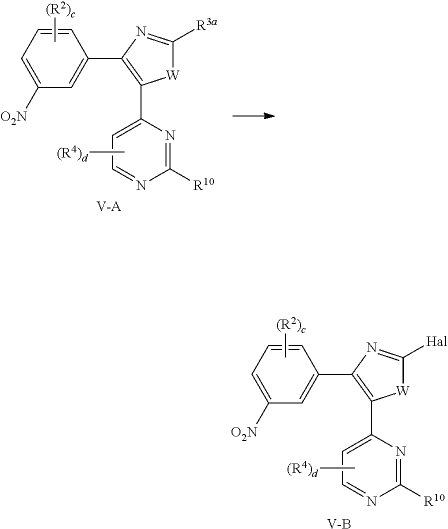

wherein Hal is halo, preferably Br; and all other variables are as defined above.

The conversion to a halo-thiazole of formula (V-B) may be carried out by reaction with for example, t-butyl nitrite and copper (II) bromide in a suitable solvent, such as tetrahydrofuran or acetonitrile, and at a temperature from −10° C. to 50° C., preferably 0° C. to 25° C. The halo-thiazole of formula (V-B), may then be reacted under a variety of conditions known to those in the art to produce different thiazole compounds of formula (V) wherein R³ can be a variety of substituents consistent with the definition of R³ in formula (I).

One example of such a reaction is similar to the method of J. Tsuji “Palladium Reagents and Catalysts: Innovations in Organic Synthesis”, Wiley, Chichester, UK, 1995, involving reaction of the halo-thiazole of formula (V-B) with a reagent capable of undergoing palladium-based coupling to prepare compounds of formula (V-C) wherein R^{3c} is alkyl, haloalkyl, alkenyl, or Ph.

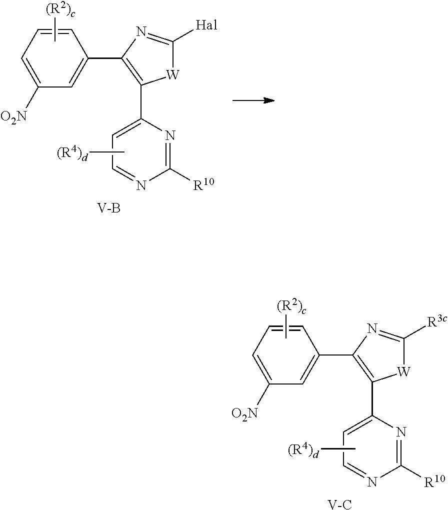

wherein Hal is halogen;
R^{3c} is alkyl, haloalkyl, alkenyl, or Ph (particularly phenyl substituted by OH, NH₂, N(H)C₁₋₃alkyl or N(C₁₋₃alkyl)₂); and
all other variables are as defined above.

For example the halo-thiazole of formula (V-B) may be reacted with a boronic acid, boronate ester, alkyl tin, alkyl zinc or Grignard reagent, in an appropriate solvent, for example tetrahydrofuran, dioxane, or dimethylformamide, in the presence of a catalyst capable of inducing such a transformation, particularly a palladium catalyst, for example palladiumdicholorobistriphenylphosphine, and at a temperature of 25-150° C., preferably 25-60° C. Those skilled in the art will recognize that these coupling reactions will often require the addition of a suitable base, such as aqueous sodium carbonate, cesium carbonate, or triethylamine and/or the addition of a suitable ligand for the palladium species, for example a trialkylphosphine or a triarylphosphine, for example triphenylphosphine.

Another example of such a reaction involves the reaction of the halo-thiazole of formula (V-B) with a reagent capable of displacing the bromide, for example an an amine, such as piperidine, methylamine, or methyl piperazine.

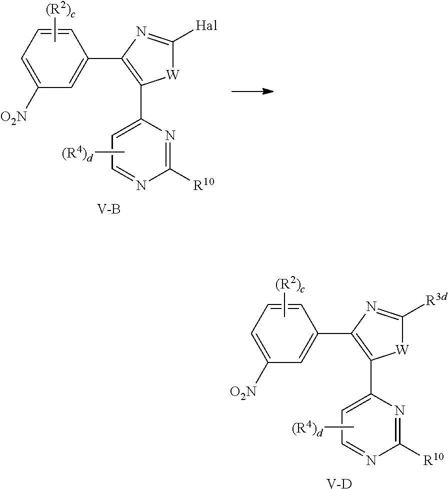

wherein Hal is halogen;

R^{3d} is selected from Het, —NR⁶R⁷, —N(R⁶)-cycloalkyl, —N(R⁶)Ph, —N(R⁶)Het, —N(R⁶)R⁵—Het, —N(R⁶)—R⁶—OR⁷, —N(R⁶)—R³—NR⁶R⁷ and —N(R⁶)—R³—S(O)_{f}R⁷; and
all other variables are as defined above.

In the case of reacting the halo-thiazole of formula (V-B) with an alcohol, the reaction is typically performed by reaction of the compound of formula (V-B) with an alkoxide, either commercially available or derived from the treatment of a suitable alcohol, such as methanol, with a base capable of accomplishing the deprotonation, for example sodium hydride. The reaction is typically carried out in a suitable solvent, such as tetrahydrofuran or dimethylformamide, at temperature of −10° C. to 90° C., particularly 25-60° C.

In the case of reacting a halo-thiazole of formula (V-B) with an amine, substituted amine (e.g., dimethylamine) or N-heterocycle (e.g., morpholine or N-methyl piperidine, the reaction is generally performed by reacting the compound of formula (V-B) with the amine, substituted amine or N-heterocycle, optionally in a suitable solvent, such as 2-propanol, dioxane, or dimethylformamide, at a temperature of 25° C. to 150° C., preferably 50-90° C., optionally in the presence of a suitable acid, for example hydrochloric acid.

According to another process of producing a substituted thiazole of formula (V), a compound of formula (IV-A) is reacted with a thioamide, for example thioacetamide, to prepare a compound of formula (V-E) wherein R^{3e} is selected from alkyl and Ph.

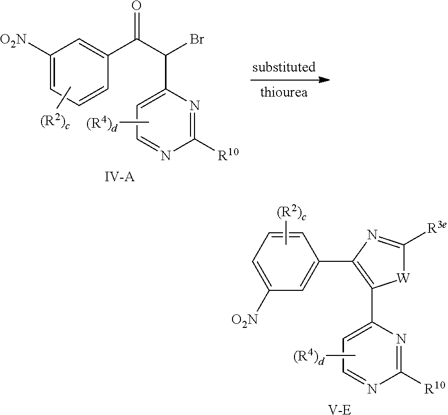

wherein R^{3e} is alkyl or Ph and all variables are as defined above.

Alkyl and aryl substituted thioamides for use in this process are commercially available or may be prepared using conventional techniques. Typically, the reaction is carried out in an appropriate solvent, for example, dichloromethane, tetrahydrofuran, dimethylformamide, N,N-dimethylacetamide, or acetonitrile, particularly dimethylformamide or N,N-dimethylacetamide, optionally in the presence of a suitable base, for example magnesium carbonate or sodium bicarbonate, and at a temperature of 35-100° C., preferably 50-70° C.

In the embodiment wherein an oxazole of formula (V) is desired wherein R³ is H, the reaction can be accomplished by reacting the compound of formula (IV-A) with formamide in the presence of an acid, such as sulfuric acid, and at a temperature of 60-150° C., preferably 100-130° C.

A substituted oxazole of formula (V-F) may be prepared from the compound of formula (IV-A).

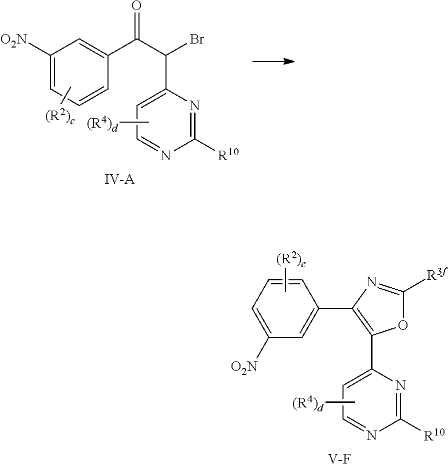

wherein R^{3f} is selected from —NR⁶R⁷, —N(R⁶)-cycloalkyl, —N(R⁶)Ph, —N(R⁶)Het, —N(R⁶)R⁵—Het, —N(R⁶)—R⁵—OR⁷, —N(R⁶)—R⁵—NR⁶R⁷, —N(H)C(O)R⁶, —N(R⁶)—C(O)—NR⁶R⁷, —N(H)SO₂R⁶, —N(R⁶)—R⁵—S(O)_{f}R⁷, and —N(R⁶)—S(O)₂—NR⁶R⁷; and
all other variables are as defined above.

The reaction may be carried out by reacting the compound of formula (IV-A) with a urea or substituted urea in an appropriate solvent, for example, dichloromethane, tetrahydrofuran, dioxane, or acetonitrile, optionally in the presence of a suitable base, for example magnesium carbonate or sodium bicarbonate, and at a temperature of 25-170° C., particularly 60-150° C. or in a microwave reactor at a temperature of 100-190° C., particularly 120-160° C. Those skilled in the art will envision substituted ureas that may be employed in the foregoing method to prepare compounds of formula (V-F) wherein R³ is as defined above. One example of a substituted urea for use in this method is N-[2-(4-morpholinyl)ethyl]urea. Suitable substituted ureas are commercially available or can be made using techniques known to those skilled in the art.

A substituted oxazole of formula (V-G), may also be prepared from a compound of formula (IV-A).

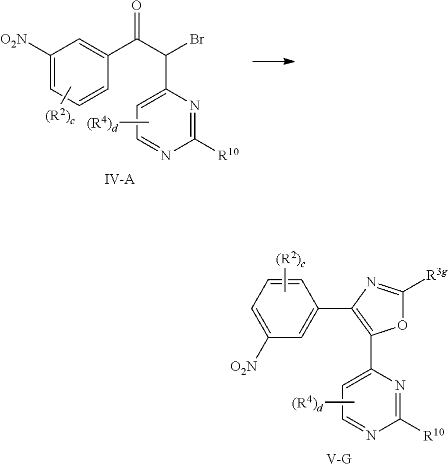

wherein R^{3g} is alkyl or haloalkyl and all other variables are as defined above.

Typically, the reaction may be carried out by reacting the compound of formula (IV-A) with an amide (i.e., a compound of formula R^{3g}—C(O)NH₂), for example acetamide, in an appropriate solvent, for example, dichloromethane, tetrahydrofuran, dimethylformamide, or acetonitrile, particularly dimethylformamide, optionally in the presence of a suitable base, for example magnesium carbonate or sodium bicarbonate, and at a temperature of 35-170° C., preferably 60-150° C. or in a microwave reactor at a temperature of 100-190° C., particularly 130-170° C. Suitable amides for use in this reaction will be apparent to those skilled in the art and are commercially available or may be prepared using convention techniques.

As will be appreciated by those skilled in the art a bromo-substituted oxazole of formula (V-H),

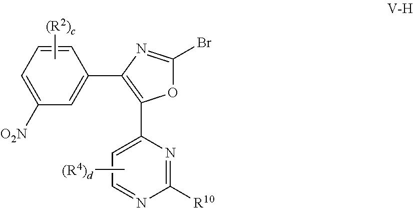

wherein all other variables are as defined above;
   may also be prepared by conversion of an oxazole of formula (V-F) (wherein R³ is an amine or substituted amino group) to the bromo analog using techniques known to those of skill in the art, including those described above.

Those of skill in the art will recognize that some of the reactions described above may be incompatible with compounds of formula (V) in which R¹⁰ is chloride. In such embodiments, the foregoing reactions may be performed using compounds of formula (V) wherein R¹⁰ is thiomethyl, and subsequently converting the thiomethyl to a more suitable leaving group, such as a sulfoxide, sulfone or chloride using techniques conventional in the art, including those described above.

Compounds of formula (IV) may be prepared by reacting a compound of formula (II) with a substituted pyrimidine of formula (III).

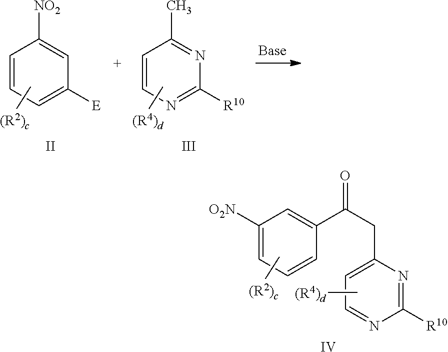

wherein all variables are as defined above.

These reactions are generally performed by reacting a compound of formula (II) and a compound of formula (III) in the presence of a suitable base capable of deprotonating a compound of formula (III), for example lithium hexamethyldisilazide (LHMDS), sodium hexamethyldisilazide, or lithium diisopropylamide, particularly LHMDS, in an appropriate solvent, such as THF, and at a temperature of from about −78° C. to about 25° C., particularly about 0° C. to about 25° C.

As noted above, the order of the foregoing steps is not critical to the practice of the present invention. In another embodiment, compounds of formula (I) wherein Y is a moiety ii or moeity iii wherein Q² is —N(H)—, may also be prepared according to Scheme 2.

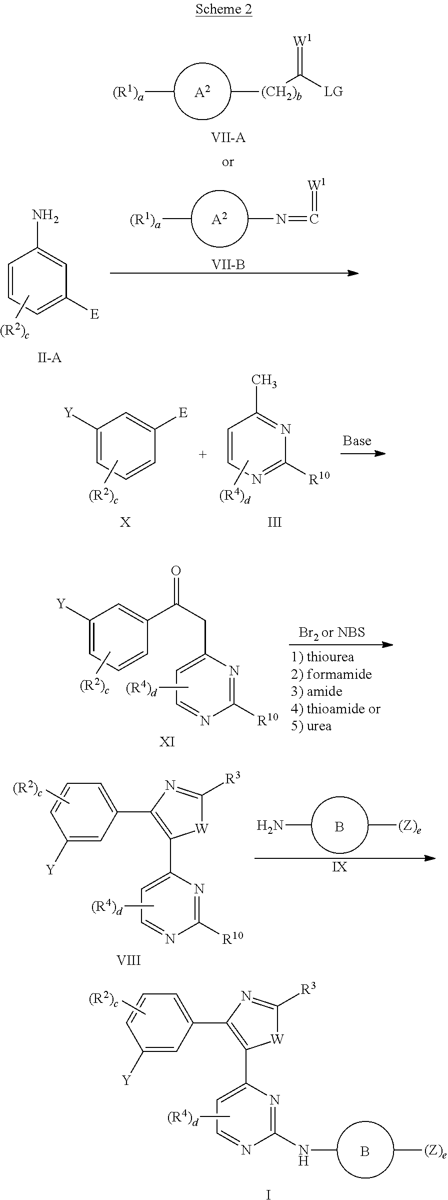

wherein:
R¹⁰ is halo (preferably chloro) or thiomethyl;
E is a suitable carboxylic ester or carboxylic ester equivalent, particularly a methyl ester, ethyl ester, or Weinreb's amide;
LG is a suitable leaving group;
Ring A² is phenyl (moiety iii) or Ring A¹ (moiety ii);
Y is a moiety ii or moiety iii wherein Q² is —N(H)—; and
all other variables are as defined above.

The process for preparing the compounds of formula (I) according to Scheme 2 (all formulas and all variables having been defined above) comprises the steps of:
a) reacting a compound of formula (II-A) with a compound of formula (VII-A) or a compound of formula (VII-B) to prepare a compound of formula (X);
b) condensing the compound of formula (X) with a substituted pyrimidine of formula (III) to prepare a compound of formula (XI);
c) reacting the compound of formula (XI) with a suitable brominating agent, followed by reacting with one of:
   i) a thiourea,
   ii) a formamide,
   iii) an amide,
   iv) a thioamide, or
   v) a urea;
   to prepare a compound of formula (VIII);
d) reacting the compound of formula (VIII) with an aniline of formula (IX) to prepare a compound of formula (I);
e) optionally converting the compound of formula (I) to a pharmaceutically acceptable salt thereof; and
f) optionally converting the compound of formula (I) or a pharmaceutically acceptable salt thereof to a different compound of formula (I) or a pharmaceutically acceptable salt thereof.

The order of the foregoing steps is not critical to the processes of the present invention and the process may be carried out using any suitable order of steps.

The reaction of the compounds of formula (VIII) with the aniline of formula (IX) is described above.

According to this process, the compound of formula (VIII) may be prepared by reacting a compound of formula (XI) with a suitable brominating agent, particularly bromine or NBS, followed by reacting with one of a thiourea, a formamide, an amide, a thioamide, or a urea (including substituted analogs thereof) in the same manner as discussed above for the reaction of a compound of formula (IV).

The compounds of formula (XI) may be prepared by condensing a compound of formula (X) with a compound of formula (III) in the same manner as described above for the condensation of a compound of formula (II) with a compound of formula (III).

A compound of formula (X) may be prepared by reacting the compound of formula (II-A) with a compound of formula (VII-A) or a compound of formula (VII-B) in the same manner as described above for the reaction of a compound of formula (VI) with a compound of formula (VII-A) or (VII-B).

Compounds of formula (II-A) may be prepared by reducing a compound of formula (II) using conventional reduction techniques, including those described above for the hydrogenation of a compound of formula (V).

As a further example, compounds of formula (I) wherein Y is moiety ii or moiety iii wherein Q² is —N(H)—, may also be prepared according to Scheme 3.

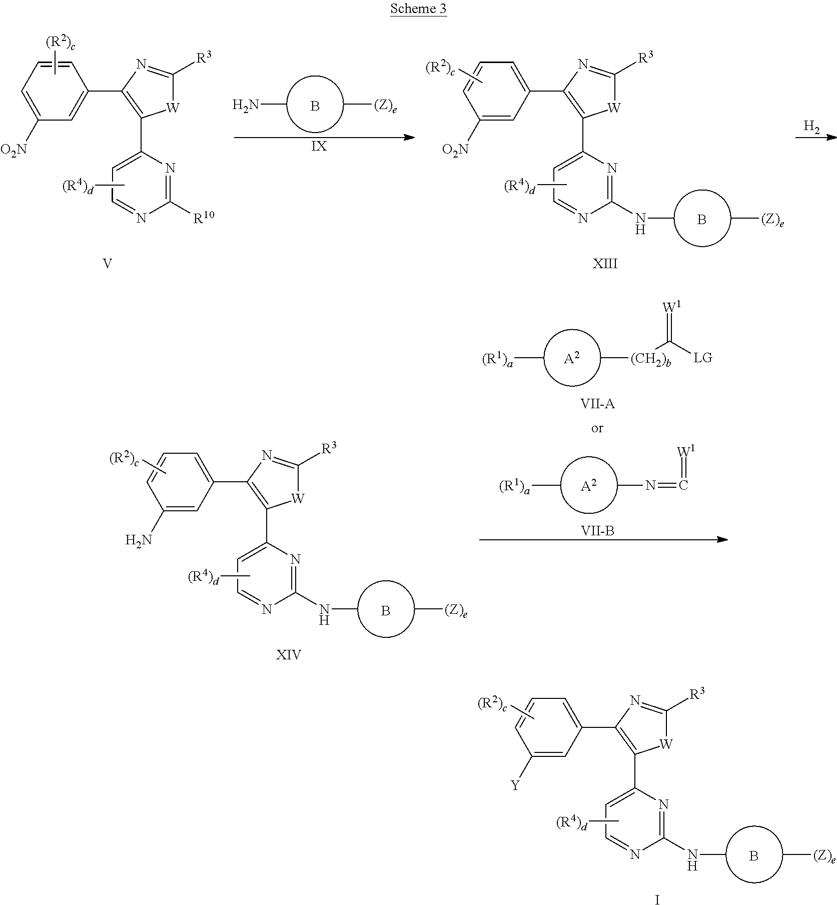

wherein:
R¹⁰ is halo (preferably chloro) or thiomethyl;
LG is a suitable leaving group;
Ring A² is phenyl (moiety iii) or Ring A¹ (moiety ii);
Y is a moiety ii or moiety iii wherein Q² is —N(H)—; and
all other variables are as defined above.

The process for preparing the compounds of formula (I) according to Scheme 3 (all formulas and all variables having been defined above) comprises the steps of:
a) reacting a compound of formula (V) with an aniline of formula (IX) to prepare a compound of formula (XIII);
b) reducing the compound of formula (XIII) to prepare a compound of formula (XIV);
c) reacting the compound of formula (XIV) with a compound of formula (VII-A) or a compound of formula (VII-B) to prepare a compound of formula (I);
d) optionally converting the compound of formula (I) to a pharmaceutically acceptable salt thereof; and
e) optionally converting the compound of formula (I) or a pharmaceutically acceptable salt thereof to a different compound of formula (I) or a pharmaceutically acceptable salt thereof.

Each of the foregoing steps may be carried out using the techniques described above for analogous reactions with different starting materials. Thus, the reaction of the compounds of formula (V) with the aniline of formula (IX) is carried out in the same manner as discussed above for the reaction of a compound of formula (VIII) with the aniline of formula (IX); the reaction of the compound of formula (XIV) with a compound of formula (VII-A) or (VII-B) is carried out in the same manner as the reaction of a compound of formula (VI) or a compound of formula (II-A) with a compound of formula (VII-A) or (VII-B); and the reducing step may be carried out in the same manner as described for the reduction of a compound of formula (V).

Compounds of formula (I) wherein Y is moiety ii or moiety iii wherein Q² is —N(H)—, may be conveniently prepared by the process outlined in Scheme 4 below.

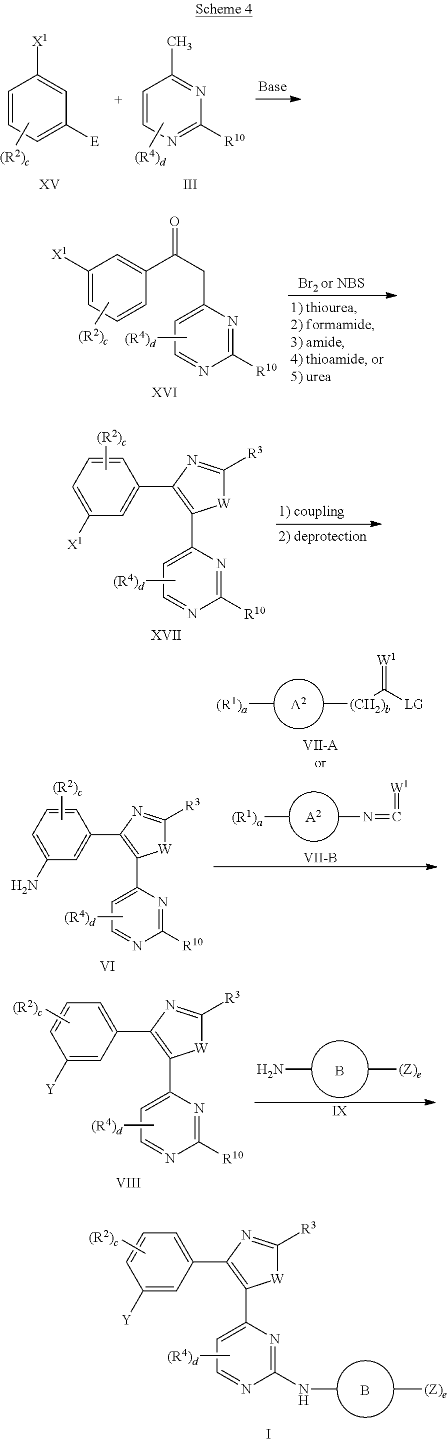

wherein:
R¹⁰ is halo (preferably chloro) or thiomethyl;
E is a suitable carboxylic ester or carboxylic ester equivalent, particularly a methyl ester, ethyl ester, or Weinreb's amide;
LG is a suitable leaving group;
Ring A² is phenyl (moiety iii) or Ring A¹ (moiety ii);
Y is a moiety ii or moiety iii wherein Q² is —N(H)—; and
X¹ is a halo, preferably a bromo, and
all other variables are as defined above.

The process for preparing compounds of formula (I) according to Scheme 4 (all formulas and all variables having been defined above), comprises the steps of:
a) condensing the compound of formula (XV) with a substituted pyrimidine compound of formula (III) to prepare a compound of formula (XVI);
b) reacting the compound of formula (XVI) with a suitable brominating agent followed by one of:
   i) a thiourea,
   ii) a formamide,
   iii) an amide,
   iv) a thioamide, or
   v) a urea;
   to prepare a compound of formula (XVII);
c) reacting the compound of formula (XVII) with an ammonia equivalent in the presence of an appropriate catalyst, optionally with subsequent deprotection, to prepare a compound of formula (VI);
d) reacting a compound of formula (VI) with a compound of formula (VII-A) or a compound of formula (VII-B) to prepare a compound of formula (VIII);
e) reacting the compound of formula (VIII) with an aniline of formula (IX) to prepare a compound of formula (I);
f) optionally converting the compound of formula (I) to a pharmaceutically acceptable salt thereof; and
g) optionally converting the compound of formula (I) or a pharmaceutically acceptable salt thereof to a different compound of formula (I) or a pharmaceutically acceptable salt thereof.

The order of the foregoing steps is not critical to the process of the present invention, and the process may be carried out using any suitable order of steps.

The reaction of the compounds of formula (VIII) with the aniline of formula (IX) and the reaction of compounds of formula (VI) with compounds of formula (VII-A) or (VII-B) are described above.

Compounds of formula (VI) may be prepared by the coupling reaction of a compound of formula (XVII) with an ammonia equivalent in the presence of an appropriate catalyst, optionally with subsequent deprotection.

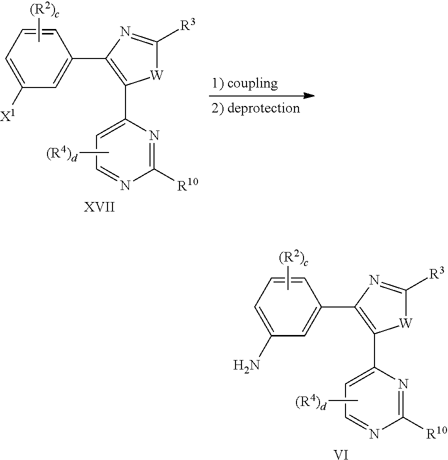

wherein all variables are as defined above.

This reaction may be accomplished in several ways. One process will involve the reaction of compound of formula (XVII), wherein X¹ is a halo, preferably a bromide, with an ammonia equivalent, such as benzophenone imine, bis(1,1-dimethylethyl) imidodicarbonate or sodium azide, in the presence of a palladium or copper catalyst capable of inducing such a transformation, for example palladiumdicholorobistriphenyl-phosphine, tris(dibenzylideneacetone)dipalladium, copper acetate, or copper iodide and at a temperature of 25-150° C., preferably 25-60° C. Those skilled in the art will recognize that these coupling reactions will often require the addition of a suitable base, such as aqueous sodium carbonate, cesium carbonate, potassium carbonate, or triethylamine and/or the addition of a suitable ligand for the palladium, for example a trialkylphosphine or a triarylphosphine, for example triphenylphosphine, or a suitable ligand for the copper, for example sodium ascorbate or 2-acetylcyclohexanone.

Those of skill in the art will recognize that the inclusion of an ammonia equivalent, such as benzophenone imine, bis(1,1-dimethylethyl) imidodicarbonate or sodium azide, will require a deprotection to give a compound of formula (VI). The deprotection conditions will be selected from the conventional methods known in the art. For example, when benzophenone imine, bis(1,1-dimethylethyl) imidodicarbonate is used as the ammonia equivalent, deprotection can be accomplished by treatment with an aqueous acid, such as acetic acid or HCl at a temperature of 25° C. to 100° C. When sodium azide is used as the ammonia equivalent, reduction to a compound of formula (VI) can be accomplished by methods well known to those of skill in the art.

Those skilled in the art will recognize that the above reactions may be incompatible with compounds of formula (XVII) when R¹⁰ is a halo, for example, chloride, or may produce mixtures. In such embodiments, those of skill in the art will recognize that the foregoing reactions can be performed with a compound of formula (XVII) in which R¹⁰ is thiomethyl to produce a compound of formula (VI) wherein R¹⁰ is a thiomethyl, and subsequently converting to a compound of formula (VI) in which R¹⁰ is halo using procedures described above. Alternatively, those of skill will recognize that appropriate choice of reaction conditions can minimize the undesired reaction and that any mixtures can be separated using conventional methods.

The compound of formula (XVII) may be prepared by reacting a compound of formula (XVI) with a suitable brominating agent, particularly bromine or NBS, followed by reacting with one of a thiourea, a formamide, an amide, a thioamide, or a urea (including substituted analogs thereof) in the same manner as discussed above for the reaction of a compound of formula (IV).

The compounds of formula (XVI) may be prepared by condensing a compound of formula (XV) with a compound of formula (III) in a the same manner as described above for the condensation of a compound of formula (II) with a compound of formula (III).

Compounds of formula (I) wherein Y is moiety ii or moiety iii wherein Q² is —N(H)—, may be conveniently prepared by the process outlined in Scheme 5.

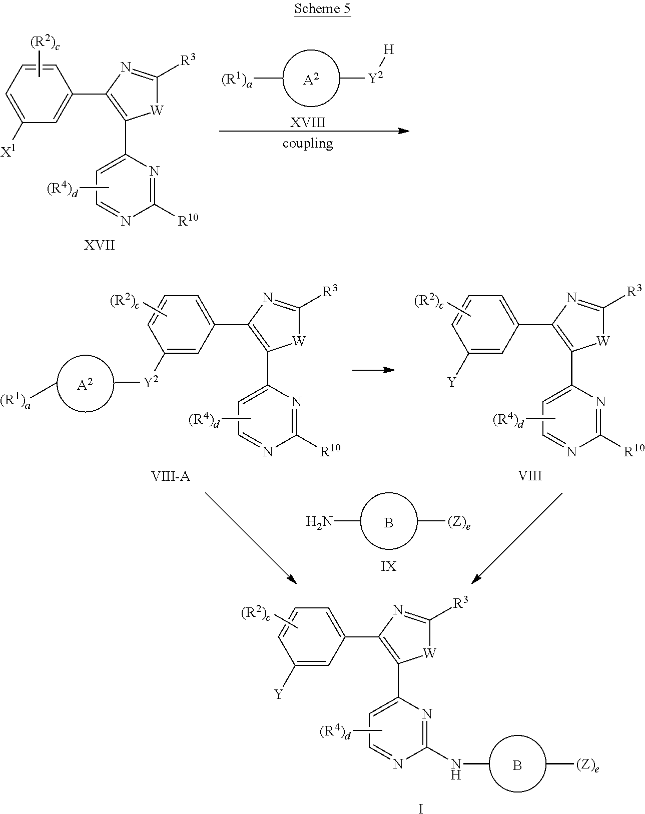

wherein:
R¹⁰ is halo(preferably chloro) or thiomethyl;
Y² is —C(O)NH, —CH₂—C(O)NH—, or —N(H)C(O)N(H)—;
Ring A² is phenyl (moiety iii) or Ring A¹ (moiety ii);
Y is a moiety ii or moiety iii wherein Q² is —N(H)—; and
X¹ is a halo, preferably a bromo, and
all other variables are as defined above.

The process for preparing compounds of formula (I) (all formulas and all variables having been defined above) according to Scheme 5, comprises the steps of:
a) reacting the compound of formula (XVII) with a compound of formula (XVIII) in the presence of an appropriate catalyst, to prepare a compound of formula (VIII-A); optionally converting a compound of formula (VIII-A) wherein Y² is —C(O)NH, —CH₂—C(O)NH—, or —N(H)C(O)N(H)— into a corresponding compound of formula (VIII) wherein Y is a moiety ii or moiety iii wherein Q² is —N(H)—;
b) reacting the compound of formula (VIII-A) or a compound of formula (VIII) with an aniline of formula (IX) to prepare a compound of formula (I);
c) optionally converting the compound of formula (I) to a pharmaceutically acceptable salt thereof; and
d) optionally converting the compound of formula (I) or a pharmaceutically acceptable salt thereof to a different compound of formula (I) or a pharmaceutically acceptable salt thereof.

The order of the foregoing steps is not critical to the process of the present invention, and the process may be carried out using any suitable order of steps. In particular, the compound of formula (VIII-A), wherein Y² is —C(O)NH, —CH₂—C(O)NH—, or —N(H)C(O)N(H)—, may be reacted with a compound of formula (IX) to prepare a compound of the invention wherein Y is a moiety ii or moiety iii wherein Q² is —N(H)-— using methods described above for the reaction of a compound of formula (VIII) with a compound of formula (IX). Compounds of formula (I) may be readily converted into a different compound of formula (I) wherein Y is moiety ii or iii using techniques known in the art.

Compounds of formula (VIII-A) may be prepared by coupling a compound of formula (XVII) with a compound of formula (XVIII).

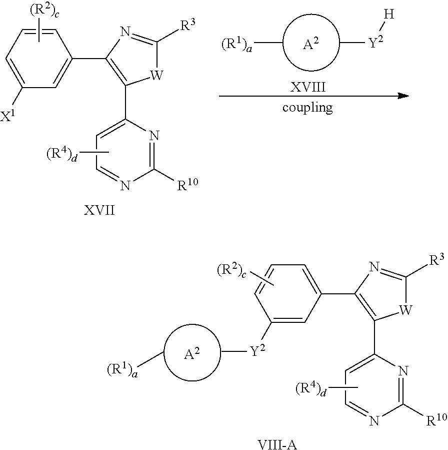

wherein all variables are defined as above.

Those skilled in the art will recognize that this reaction may be accomplished in several ways. One process will involve the reaction of compound of formula (XVII), wherein X¹ is a halo, preferably a bromide, with a compound of formula (XVIII) wherein Y² is —C(O)NH, —CH₂—C(O)NH—, or —N(H)C(O)N(H)— in the presence of a palladium or copper catalyst capable of inducing such a transformation, for example palladiumdicholorobistriphenylphosphine, tris(dibenzylideneacetone)dipalladium, copper acetate, or copper iodide and at a temperature of 25-150° C., preferably 25-60° C. Those skilled in the art will recognize that these coupling reactions will often require the addition of a suitable base, such as aqueous sodium carbonate, cesium carbonate, potassium carbonate, or triethylamine and/or the addition of a suitable ligand for the palladium, for example a trialkylphosphine or a triarylphosphine, for example triphenylphosphine, or a suitable ligand for the copper, for example sodium ascorbate or 2-acetylcyclohexanone.

Those skilled in the art will recognize that the above reactions may be incompatible with compounds of formula (XVII) when R¹⁰ is a halo, for example, chloride, or may produce mixtures. In such embodiments, those of skill in the art will recognize that the foregoing reactions can be performed with a compound of formula (XVII) in which R¹⁰ is thiomethyl to produce a compound of formula (VIII-A) wherein R¹⁰ is a thiomethyl, and subsequently converting to a compound of formula (VIII-A) in which R¹⁰ is halo using procedures described above. Alternatively, those of skill will recognize that appropriate choice of reaction conditions can minimize the undesired reaction and that any mixtures can be separated using conventional methods.

When a compound of formula (VIII) wherein Y is moeity ii or iii wherein W¹ is S, is desired, the coupling reaction can be performed with a compound of formula (VIII-A) wherein Y² is —C(O)NH— or —NHC(O)N(H)—. The compound produced in this reaction can then be converted to the corresponding compound of formula (VIII) wherein Y is moeity ii or iii wherein W¹ is S, and then subsequently converted to a corresponding compound of formula (I). Techniques conventional to those skilled in the art such as, for example by the use of Lawesson's reagent, may be employed to effect this conversion.

Compounds of formula (XVIII) are commercially available or may be prepared using conventional techniques.

Processes for preparing compounds of formula (XVII) are described above.

In a further embodiment, compounds of formula (I) wherein Y is moiety i (i.e., compounds of formula (I-i)) may be prepared according to Scheme 6.

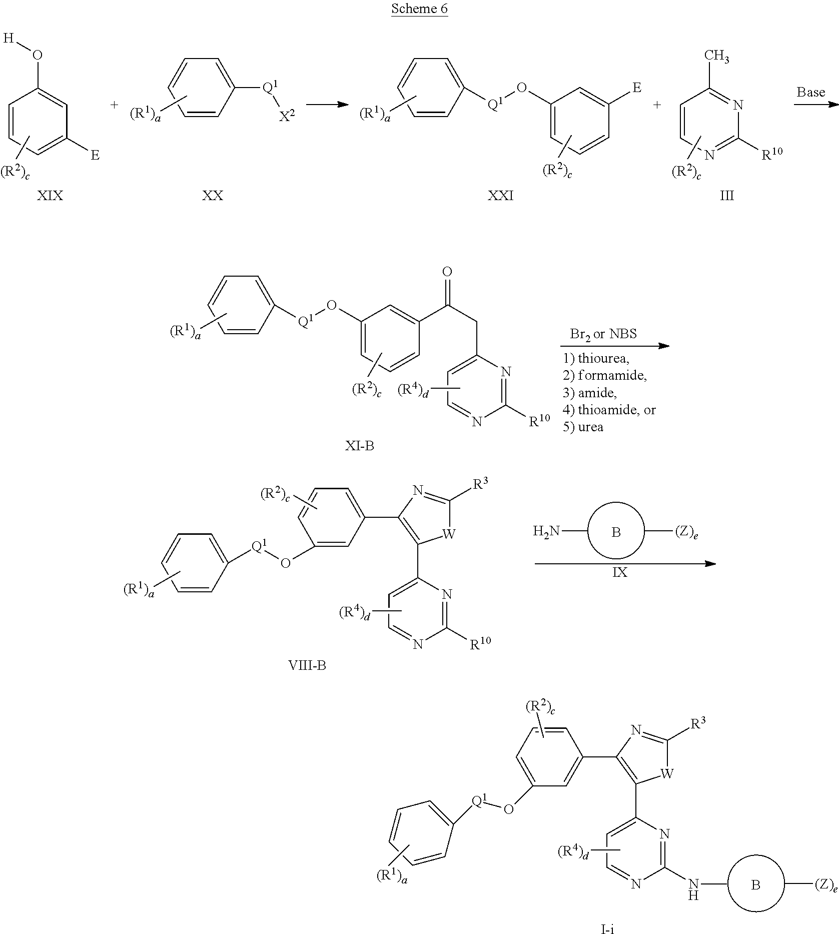

wherein:
E is a suitable carboxylic ester or carboxylic ester equivalent, particularly a methyl ester, ethyl ester, or Weinreb's amide;
X² is halo, tosylate or mesylate (particularly bromo);
R¹⁰ is halo (preferably chloro) or thiomethyl; and
all other variables are as defined above.

In general, the process for preparing compounds of formula (I) wherein Y is moiety i according to Scheme 6, comprises the steps of:

a) reacting a compound of formula (XIX) with a compound of formula (XX) to prepare a compound of formula (XXI);
b) condensing the compound of formula (XXI) with a substituted pyrimidine of formula (III) to prepare a compound of formula (XI-B);
c) reacting the compound of formula (XI-B) a suitable brominating agent, followed by reacting with one of:
i) a thiourea,
ii) a formamide,
iii) an amide,
iv) a thioamide, or
v) a urea;
   to prepare a compound of formula (VIII-B);
   d) reacting the compound of formula (VIII-B) with an aniline of formula (IX) to prepare a compound of formula (I);
   e) optionally converting the compound of formula (I) to a pharmaceutically acceptable salt thereof; and
   f) optionally converting the compound of formula (I) or a pharmaceutically acceptable salt thereof to a different compound of formula (I) or a pharmaceutically acceptable salt thereof.

The order of the foregoing steps is not critical to the process of the present invention, and the process may be carried out using any suitable order of steps.

The reaction of a compound of formula (VIII-B) with an aniline of formula (IX) to prepare a compound of formula (I) is carried out in the same manner as described above for the reaction of a compound of formula (VIII) with an aniline of formula (IX). The reaction of a compound of formula (XI-B) a suitable brominating agent, particularly bromine or NBS, followed by reaction with one of a thiourea, a formamide, an amide, a thioamide or a urea (including substituted analogs thereof) is carried out in the same manner as described above for the same reaction with a compound of formula (XI).

According to this method, the compound of formula (XI) is prepared by condensing the compound of formula (XXI) with a substituted pyrimidine of formula (III). This reaction may be carried out in the same manner as the condensation of a compound of formula (II) or a compound of formula (X) with a substituted pyrimidine of formula (III).

The compound of formula (XXI) is prepared by reacting a compound of formula (XIX) with a compound of formula (XX).

This reaction is generally carried out in a suitable solvent, such as dimethylformamide or tetrahydrofuran, in the presence of a base, for example potassium carbonate, sodium carbonate, or triethyl amine, and at a temperature of from about 10 to about 100° C., preferably from about 25 to about 50° C. Those of skill in the art will appreciate that the foregoing description represents one of many available conditions to form such ether bonds. Other examples of suitable reaction conditions are reported in R. Larock “Comprehensive Organic Transformations” VCH, New York 1989.

Phenols of formula (XIX) are generally commercially available or may be prepared using conventional techniques.

Compounds of formula (XX) are commercially available or may be prepared using conventional techniques. One example of a suitable compound of formula (XX) is benzyl bromide.

In another embodiment, compounds of formula (I) wherein Y is a moiety iii and Q² is a bond, i.e., a compound of formula (I-iii-a) or (I-iii-b), may be prepared according to Scheme 7.

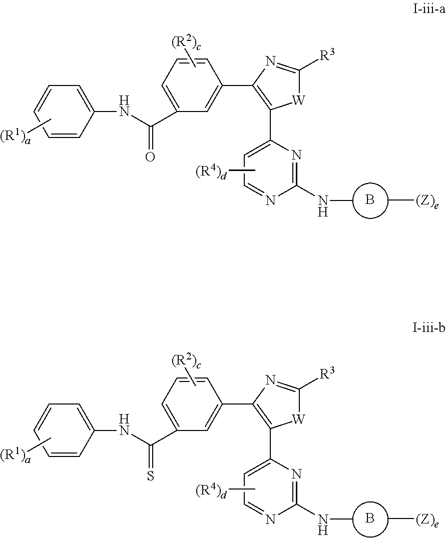

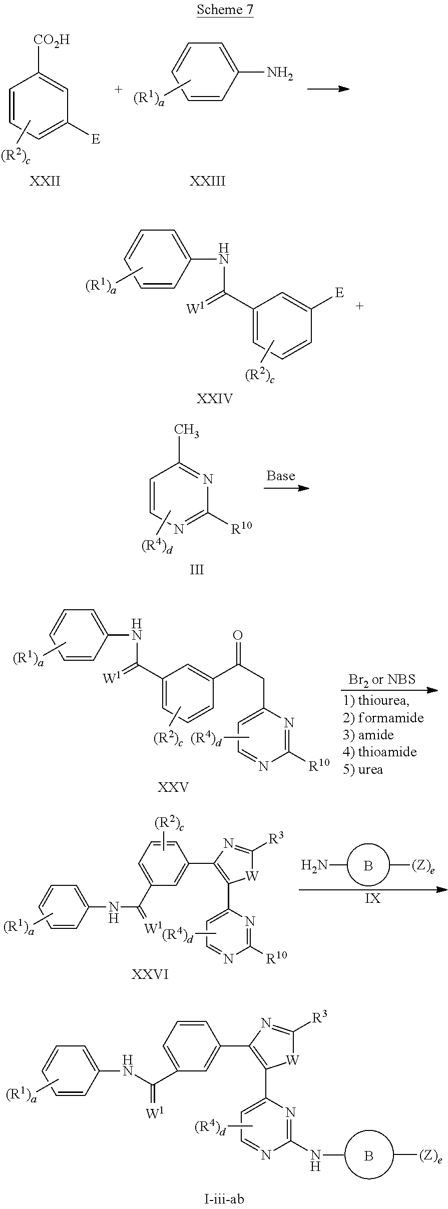

wherein:
E is a suitable carboxylic ester or carboxylic ester equivalent, particularly a methyl ester, ethyl ester, or Weinreb's amide;
R¹⁰ is halo (preferably chloro) or thiomethyl; and
all other variables are as defined above.

In general, the process for preparing compounds of formula (I) wherein Y is moiety iii and Q² is a bond, comprises reacting the compound of formula (XXVI) with an aniline of formula (IX) to prepare a compound of formula (I).

More specifically, the process for preparing compounds of formula (I) wherein Y is moiety iii and Q² is a bond (all formulas and all variables having been defined above), according to Scheme 7 comprises the steps of:
a) reacting the compound of formula (XXII) with a compound of formula (XXIII) to prepare a compound of formula (XXIV);
b) condensing the compound of formula (XXIV) with a substituted pyrimidine of formula (III) to prepare a compound of formula (XXV);
c) reacting the compound of formula (XXV) with a suitable brominating agent, particularly bromine or NBS, followed by reacting with one of:
   i) a thiourea,
   ii) a formamide,
   iii) an amide,
   iv) a thioamide, or
   v) a urea;
      to prepare a compound of formula (XXVI);
d) reacting the compound of formula (XXVI) with an aniline of formula (IX) to prepare a compound of formula (I-iii-ab);
e) optionally converting the compound of formula (I-iii-ab) to a pharmaceutically acceptable salt thereof; and
f) optionally converting the compound of formula (I-iii-ab) or a pharmaceutically acceptable salt thereof to a different compound of formula (I-iii-ab) or a pharmaceutically acceptable salt thereof.

The order of the foregoing steps is not critical to the process of the present invention, and the process may be carried out using any suitable order of steps.

The condensation of a compound of formula (XXIV) with a substituted pyrimidine of formula (III), the reaction of a compound of formula (XXV) with a suitable brominating agent followed by reaction with one of a thiourea, a formamide, an amide, a thioamide or a urea (including substituted analogs thereof), and the reaction of a compound of formula (XXVI) with an aniline of formula (IX) to prepare a compound of formula (I-iii-ab), are analogous to reactions described above.

According to this process, the compound of formula (XXIV) is prepared by reacting a compound of formula (XXII) with a compound of formula (XXIII). This reaction may be carried out by first reacting the carboxylic acid of formula (XXII) with an appropriate chlorinating agent, for example oxalyl chloride or thionyl chloride in a suitable solvent, such as dichloromethane or toluene, optionally with the addition of dimethylformamide, and at a temperature of from about 0° C. to about 50° C., preferably from about 10° C. to about 35° C. The chlorination reaction results in the acyl chloride analog of formula (XXII) which is then combined with a compound of formula (XXIII) in a suitable solvent, such as dichloromethane or tetrahydrofuran, optionally in the presence of a base, such as pyridine or triethylamine, optionally in the presence of a suitable catalyst, for example dimethylaminopyridine, and at a temperature of from about 25° C. to about 120° C., preferably from about 25° C. to about 60° C. One skilled in the art will appreciate that the foregoing description represents only one of many available reaction conditions which would be suitable for the formation of the amide bond. Other suitable reaction conditions are conventional in the art and include those in R. Larock “Comprehensive Organic Transformations”; VCH, New York, 1989.

Carboxylic acid compounds of formula (XXII) and compounds of formula (XXIII) are commercially available or may be prepared using conventional techniques.

In another embodiment, compounds of formula (I) wherein Y is moiety i, ii or iii, may be prepared according to the process outlined in Scheme 8.

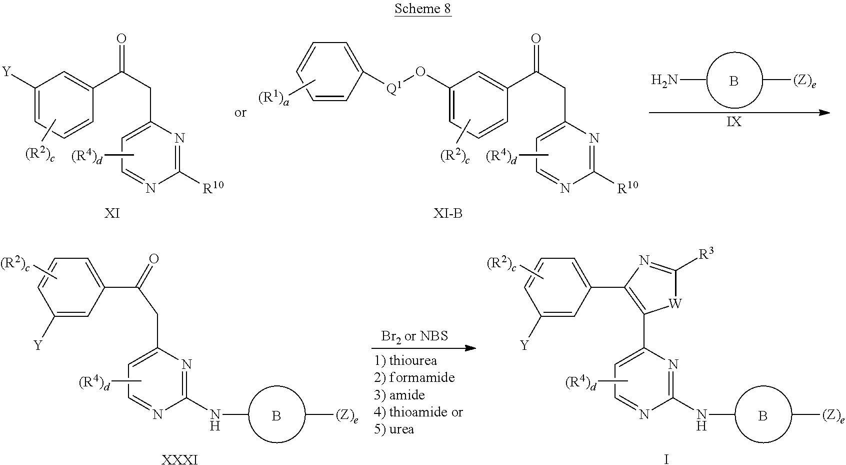

wherein:
R¹⁰ is halo (preferably chloro) or thiomethyl; and
all other variables are as defined above.

The process for preparing compounds of formula (I) (all formulas and all variables having been defined above) according to Scheme 8 comprises the steps of:

a) reacting the compound of formula (XI) or (XI-B) with an aniline of formula (IX) to prepare a compound of formula (XXXI);
b) reacting the compound of formula (XXXI) with a suitable brominating agent, particularly bromine or NBS, followed by reaction with one of:
i) a thiourea,
ii) a formamide,
iii) an amide,
iv) a thioamide, or
v) a urea;
   to prepare a compound of formula (I);
   c) optionally converting the compound of formula (I) to a pharmaceutically acceptable salt thereof; and
   d) optionally converting the compound of formula (I) or a pharmaceutically acceptable salt thereof to a different compound of formula (I) or a pharmaceutically acceptable salt thereof.

As will be apparent to those skilled in the art, the order of the foregoing steps is not critical to the process of the present invention, and the process may be carried out using any suitable order of steps.

The compound of formula (XXXI) is reacted with a suitable brominating agent, particularly bromine or NBS, followed by reaction with one of a thiourea, a formamide, an amide, a thioamide, or a urea (including substituted analogs thereof), to prepare a compound of formula (I) in a manner analogous to the procedure described above for the reaction of the compound of formula (IV) to prepare a compound of formula (V).

The compound of formula (XI) or (XI-B) is reacted with an aniline of formula (IX) to prepare the compound of formula (XXXI) in a procedure analogous to the reaction of the compound of formula (VIII) with an aniline of formula (IX) to prepare the compound of formula (I). It will be recognized by those of skill in the art that the aniline of formula (IX) might also condense with the ketone functionality of a compound of formula (XI) or (XI-B) to form the corresponding imine or enamine. A compound of formula (XXXI) can be readily produced from the corresponding imine or enamine by reaction with an appropriate acid, such as acetic acid or HCl, optionally in a solvent such as dichloromethane of tetrahydrofuran and at a temperature of about 0° C. to about 50° C., preferably at ambient temperature.

Compounds of formula (XI) and (XI-B) may be prepared as described above.

In another embodiment, compounds of the invention may be prepared according to the process outlined in Scheme 9.

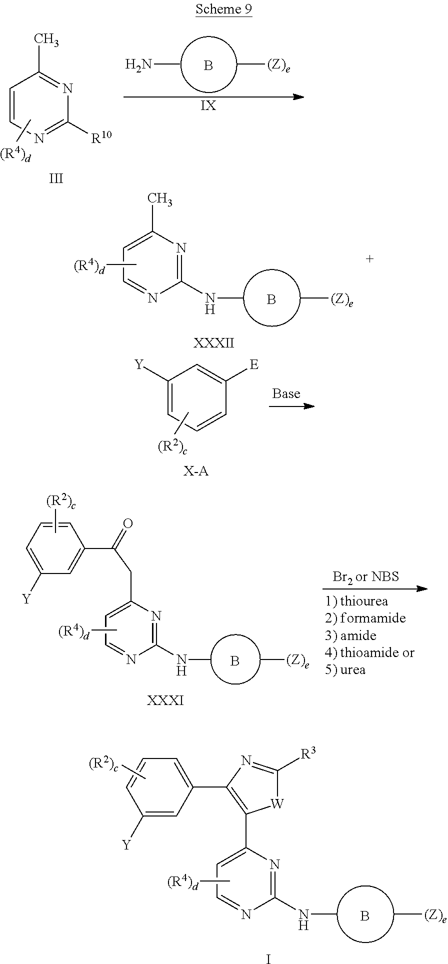

wherein:
R¹⁰ is halo (preferably chloro) or thiomethyl;
Y is moiety i, ii or iii; and
all other variables are as defined above.

In general, the process for preparing compounds of formula (I) according to Scheme 9 comprises the steps of:

a) reacting the compound of formula (III) with an aniline of formula (IX) to prepare a compound of formula (XXXII);
b) condensing the pyrimidine of formula (XXXII) with a compound of formula (X-A) to prepare a compound of formula (XXXI);
c) reacting the compound of formula (XXXI) with a suitable brominating agent followed by reaction with one of:
i) a thiourea,
ii) a formamide,
iii) an amide,
iv) a thioamide, or
v) a urea;
   to prepare a compound of formula (I);
   d) optionally converting the compound of formula (I) to a pharmaceutically acceptable salt thereof; and
   e) optionally converting the compound of formula (I) or a pharmaceutically acceptable salt thereof to a different compound of formula (I) or a pharmaceutically acceptable salt thereof.

As will be apparent to those skilled in the art, the order of the foregoing steps is not critical to the process of the present invention, and the process may be carried out using any suitable order of steps.

The compound of formula (XXXI) is reacted with a suitable brominating agent, particularly bromine or NBS, followed by reaction with one of a thiourea, a formamide, an amide, a thioamide, or a urea (including substituted analogs thereof), to prepare a compound of formula (I) in a manner analogous to the procedure described above for the reaction of the compound of formula (IV) to prepare a compound of formula (V).

The compound of formula (XXXI) is prepared by condensing the compound of formula (X-A) with a substituted pyrimidine of formula (III). This reaction may be carried out in the same manner as the condensation of a compound of formula (II) or a compound of formula (X) with a substituted pyrimidine of formula (III). Those of skill in the art will recognize that this reaction may require additional base.

The preparation of a compound of formula (X-A) is described above in the form of processes for preparing compounds of formula (X) (wherein Y is a moiety ii or moiety iii wherein Q² is —N(H)—) and compounds of formula (XXI)

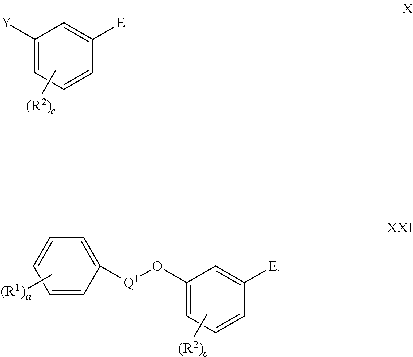

The pyrimidine of formula (III) is reacted with an aniline of formula (IX) to prepare the compound of formula (XXXII) in a procedure analogous to the reaction of the compound of formula (VIII) with an aniline of formula (IX) to prepare the compound of formula (I).

It will be appreciated by those skilled in the art that the optimal choice of the reaction sequence employed to prepare a particular compound of the invention may depend upon the specific compound of the invention that is desired as well as the preference and availability of starting materials.

As will be apparent to those skilled in the art, a compound of the invention may be converted to another compound of the invention using techniques well known in the art. For example, compounds of the invention may be modified using conventional techniques to modify or diversify the groups defined by the variable R³ and thereby provide different compounds of the invention. Specifically, a compound of formula (I-1) (wherein R³ is —NH₂) may be converted to a compound of formula (I-2) by reductive amination of the amine with acetone and sodium cyanoborohydride.

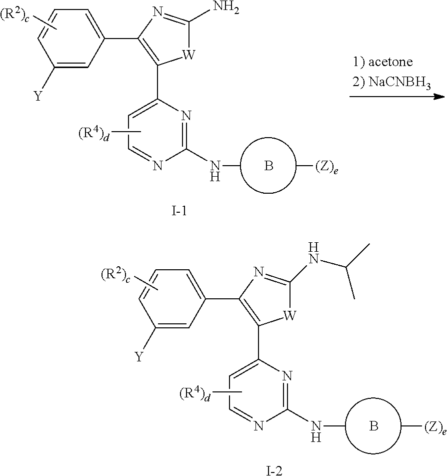

wherein, in this and the following examples of transformations, all variables are as defined herein.

A compound of formula (I-1) may also be converted to a compound of formula (I-3) by reacting with mesyl chloride.

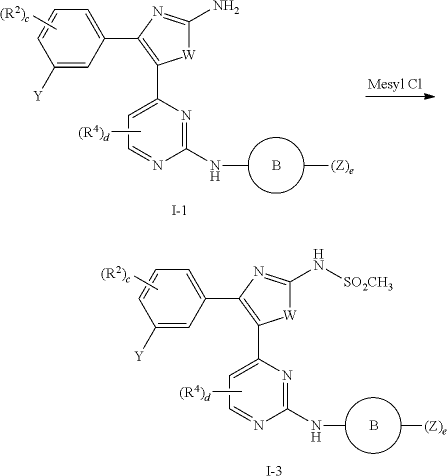

An ester compound of formula (I-4) may be converted to an alcohol compound of formula (I-5) by reacting with methanol and a suitable base such as sodium methoxide.

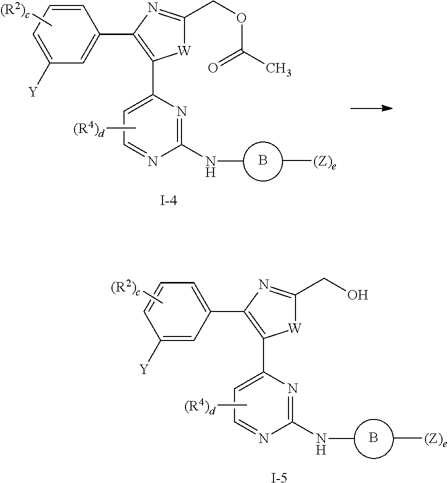

Compounds of the invention may also be diversified in the position defined by Z using conventional techniques to convert a compound of the invention to a different compound of the invention. For example, a compound of formula (I-6) may be converted to a compound of formula (I-7) by reacting with morpholine.

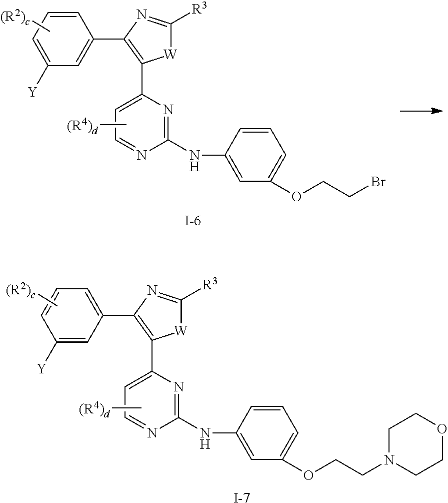

A compound of formula (I-9) may be converted to a compound of formula (I-10) by reacting with acetic anhydride.

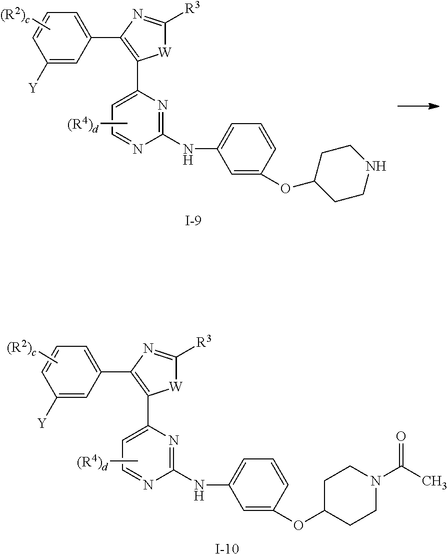

Compounds of formula (I) wherein Y is a moiety ii or iii, may also be diversified in the position defined by W¹ using conventional techniques, to convert a compound of the invention to a different compound of the invention. For example, one method of converting a compound of formula (I) wherein Y is a moiety ii, b is 0 and W¹ is ═O (i.e., a compound of formula (I-11)) to another compound of formula (I) comprises treating a compound of formula (I-11) with Lawesson's reagent.

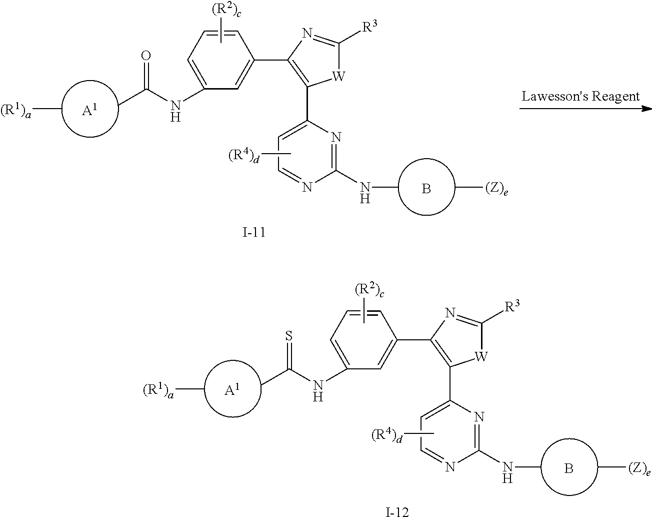

Suitable conditions for this reaction will be apparent to those skilled in the art of organic synthesis.

A compound of the invention wherein the moiety Ring B-(Z)ₑ is a tetrahydroisoquinoline group wherein the tetrahydroisoquinoline amine is a secondary amine may be converted into another compound of the invention wherein the amine is a tertiary amine bearing a methyl group. This transformation may be accomplished through a reductive amination procedure. Procedures for reductive amination are well known in the literature and include, for example, stirring the secondary amine bearing compound in a suitable solvent in the presence of aqueous formaldehyde and sodium triacetoxyborohydride and catalytic acid. Examples of suitable solvents include methylene chloride or N,N-dimethylformamide. An example of a suitable acid is acetic acid.

Based upon this disclosure and the examples contained herein one skilled in the art can readily convert a compound of formula (I) or a pharmaceutically acceptable salt thereof into a different compound of formula (I), or a pharmaceutically acceptable salt thereof.

The present invention also provides radiolabeled compounds of formula (I) and biotinylated compounds of formula (I) and solid-support-bound versions thereof, i.e. a compound of formula (I) having a radiolabel or biotin bound thereto. Radiolabeled compounds of formula (I) and biotinylated compounds of formula (I) can be prepared using conventional techniques. For example, radiolabeled compounds of formula (I) can be prepared by reacting the compound of formula (I) with tritium gas in the presence of an appropriate catalyst to produce radiolabeled compounds of formula (I). In one embodiment, the compounds of formula (I) are tritiated.

The radiolabeled compounds of formula (I) and biotinylated compounds of formula (I) are useful in assays for the identification of compounds which inhibit at least one Raf family kinase and/or at least one ErbB family kinase, for the identification of compounds for the treatment of a condition capable of being treated with a Raf inhibitor or an ErbB inhibitor, e.g., for the treatment of neoplasms susceptible to treatment with a Raf inhibitor or an ErbB inhibitor. The present invention also provides an assay method for identifying such compounds, which method comprises the step of specifically binding a radiolabeled compound of the invention or a biotinylated compound of the invention to the target protein or cellular homogenate. More specifically, suitable assay methods will include competition binding assays. The radiolabeled compounds of the invention and biotinylated compounds of the invention and solid-support-bound versions thereof, can also be employed in assays according to the methods conventional in the art.

The following examples are intended for illustration only and are not intended to limit the scope of the invention in any way. The invention is defined by the claims which follow.

### EXAMPLES

As used herein, the symbols and conventions used in these processes, schemes and examples are consistent with those used in the contemporary scientific literature, for example, the Journal of the American Chemical Society or the Journal of Biological Chemistry. Standard single-letter or three-letter abbreviations are generally used to designate amino acid residues, which are assumed to be in the L-configuration unless otherwise noted. Unless otherwise noted, all starting materials were obtained from commercial suppliers and used without further purification. Specifically, the following abbreviations may be used in the examples and throughout the specification:

atm (atmosphere);
g (grams);
mg (milligrams);
h (hour(s));
min (minutes);

### Hz (Hertz);

MHz (megahertz);
i. v. (intravenous);
L (liters);
mL (milliliters);
μL (microliters);
M (molar);
mM (millimolar);
mol (moles);
mmol (millimoles);
mp (melting point);
psi (pounds per square inch);
rt (room temperature);
TLC (thin layer chromatography);
Tᵣ (retention time);
RP (reverse phase;
H₂ (hydrogen);
N₂ (nitrogen)
Ac (acetyl);
Ac₂O (acetic anhydride);
ATP (adenosine triphosphate);
BOC (tert-butyloxycarbonyl);
BSA (bovine serum albumin)
CHCl₃ (chloroform);
mCPBA (meta-chloroperbenzoic acid);
DCC (dicyclohexylcarbodiimide);
DCE (dichloroethane);
DCM (CH₂Cl₂; dichloromethane);
DMA (dimethyl acetamide);
DMAP (4-dimethylaminopyridine);
Na₂SO₄ (sodium sulfate);

### NBS is N-bromosuccinimide;

NH₄OH (ammonium hydroxide);
TEA (triethylamine);
TFA (trifluoroacetic acid);
DME (1,2-dimethoxyethane);
DMEM (Dulbecco's modified Eagle medium);
DMF (N, dimethylformamide);
DMSO (dimethylsulfoxide);
EDC (ethylcarbodiimide hydrochloride);
EDTA (ethylenediaminetetraacetic acid);
Et (ethyl; —CH₂CH₃)
EtOH (ethanol);
EtOAc (ethyl acetate);
FMOC (9-fluorenylmethoxycarbonyl);
HCl (hydrochloric acid)
HEPES (4-(2-hydroxyethyl)-1-piperazine ethane sulfonic acid);
HOAc (acetic acid);
HPLC (high pressure liquid chromatography);
i-PrOH (isopropanol);
K₂CO₃ (potassium carbonate);
KOH (potassium hydroxide);
LHMDS (lithium hexamethyldisilazide);
LiOH (lithium hydroxide);
LiOH—H₂O (lithium hydroxide monohydrate);
Me (methyl; —CH₃)
MeOH (methanol);
MgCO₃ (magnesium carbonate);
MgSO₄ (magnesium sulfate);
Na₂CO₃ (sodium carbonate);
NaHCO₃ (sodium bicarbonate);
NaH (sodium hydride)
THF (tetrahydrofuran);
TIPS (triisopropylsilyl);
TMS (trimethylsilyl); and
TMSE (2-(trimethylsilyl)ethyl).

All references to ether are to diethyl ether; brine refers to a saturated aqueous solution of NaCl. Unless otherwise indicated, all temperatures are expressed in ° C. (degrees Centigrade). All reactions are conducted under an inert atmosphere at rt unless otherwise noted.

¹H-NMR spectra were recorded on a Varian VXR-300, a Varian Unity-300, a Varian Unity-400 instrument, a General Electric QE-300, a Bruker 300, or a Bruker 400. Chemical shifts are expressed in parts per million (ppm, δ units). Coupling constants are in units of hertz (Hz). Splitting patterns describe apparent multiplicities and are designated as s (singlet), d (doublet), t (triplet), q (quartet), m (multiplet), br (broad).

Low-resolution mass spectra (MS) were recorded on a JOEL JMS-AX505HA, JOEL SX-102, a SCIEX-APIiii, a Finnegan MSQ, Waters SQD, Waters ZQ, or a Finnegan LCQ spectrometer; high resolution MS were obtained using a JOEL SX-102A spectrometer. All mass spectra were taken under electrospray ionization (ESI), chemical ionization (CI), electron impact (EI) or by fast atom bombardment (FAB) methods. Infrared (IR) spectra were obtained on a Nicolet 510 FT-IR spectrometer using a 1-mm NaCl cell. All reactions were monitored by thin-layer chromatography on 0.25 mm E. Merck silica gel plates (60F-254), visualized with UV light, 5% ethanolic phosphomolybdic acid or p-anisaldehyde solution or mass spectrometry (electrospray or AP). Flash column chromatography was performed on silica gel (230-400 mesh, Merck) or using automated silica gel chromatography (Isco, Inc. Sq 16× or 100 sg Combiflash).

Reported HPLC retention times (RT) were obtained on a Waters 2795 instrument attached to a Waters 996 diode array detector reading 210-500 nm. The column used was a Synergi Max-RP (50×2 mm) model #00B-4337-B0, Solvent gradient was 15% MeOH:water to 100% MeOH (0.1% formic acid) over 6 min. Flow rate was 0.8 mL/min. Injection volume was 3 microliters.

### Example 1

### 2,6-Difluoro-N-(3-{2-{[2-(4-morpholinyl)ethyl]amino}-5-[2-(1,2,3,4-tetrahydro-7-isoquinolinylamino)-4-pyrimidinyl]-1,3-thiazol-4-yl}phenyl)benzamide

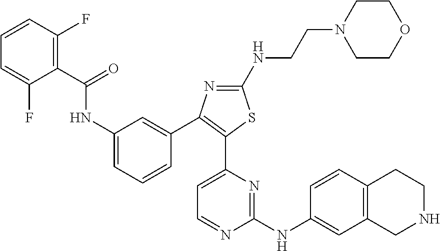

### Step A: 2-Chloro-4-methylpyrimidine

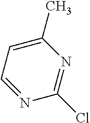

To a vigorously stirred slurry containing 50.0 g (0.31 mol) of 2,6-dichloro-4-methylpyrimidine, 250 mL of EtOH and 250 mL of water was added 41 g (0.63 mol) of zinc dust, followed by 0.78 g (3.08 mmol) of iodine. The reaction mixture was heated at 70° C. for 4 h, then cooled and filtered. The EtOH was removed under reduced pressure and the mixture was extracted with DCM. The combined organic layers were dried over MgSO₄, filtered, and the solvent was removed under reduced pressure. The residue was subjected to silica gel chromatography, eluting with a hexane/DCM mixture, to afford 20.6 g (53%) of the title compound of Step A as a white solid: ¹H-NMR (d⁶-DMSO, 400 MHz) δ 8.59 (d, 1H, J=4.9 Hz), 7.44 (d, 1H, J=4.9 Hz), and 3.29 (s, 3H); ESIMS: 129.11 (M+H⁺).

### Step B: Ethyl 3-{[(2,6-difluorophenyl)carbonyl]amino}benzoate

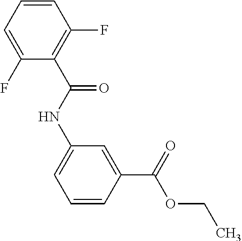

To a solution containing 5.0 g (30.3 mmol) of ethyl 3-aminobenzoate and 100 mL of DCM at 0° C. was added 3.8 mL (30.3 mmol) of 2,6-difluorobenzoyl chloride, followed by 4.7 mL (33.3 mmol) of TEA. The reaction mixture was allowed to stir for 15 min, then diluted with DCM and successively washed with 10% aqueous HCl, 10% aqueous NaOH, and brine. The organic layers were dried over MgSO₄, filtered, and concentrated under reduced pressure to afford 8.8 g (95%) of the title compound of Step B as an off-white solid: ¹H-NMR (d⁶-DMSO, 400 MHz) δ 11.02 (s, 1H), 8.34 (dd, 1H, J=1.8 and 1.8 Hz), 7.92 (dd, 1H, J=8.1 and 1.3 Hz), 7.72 (d, 1H, J=7.7 Hz), 7.56-7.63 (m, 1H), 7.51 (dd, 1H, J=7.9 and 7.9 Hz), 7.25 (dd, 2H, J=8.1 and 8.1 Hz), 4.31 (q, 2H, J=7.1 Hz), and 1.31 (t, 3H, J=7.1 Hz); ESIMS: 328.12 (M+Na⁺).

### Step C: N-{3-[(2-Chloro-4-pyrimidinyl)acetyl]phenyl}-2,6-difluorobenzamide

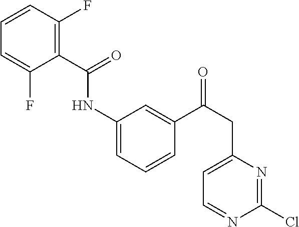

To a solution containing 5.0 g (16.3 mmol) of ethyl 3-{[(2,6-difluorophenyl)carbonyl]amino}benzoate, 11.2 mL (18 mmol) of a 1.6 M solution of 2-chloro-4-methylpyrimidine in THF, and 10 mL of THF at 0° C. was added 36 mL (36 mmoL) of a 1.0 M soln of LHMDS in THF. The reaction mixture was allowed to warm to rt and stirred for 13 h, then an additional 5 mL of a 1.6 M solution of 2-chloro-4-methylpyrimidine in THF and 10 mL of a 1.0 M solution of LHMDS in THF was added. The reaction mixture was allowed to stir for an additional 8 h, then quenched by the addition of 10% aqueous HCl and extracted with DCM. The combined organic layers were dried over MgSO₄ and filtered, and the solvent was removed under reduce pressure. The residue was subjected to silica gel chromatography, eluting with a hexane/EtOAc mixture, to give 2.5 g (40%) of the title compound of Step C as an off white solid that exists as a mixture of ketone and enol tautomers: ¹H-NMR (d⁶-DMSO, 400 MHz) ketone: δ 11.04 (s, 1H), 8.73 (d, 1H, J=4.9 Hz), 8.30 (s, 1H), 7.96 (d, 1H, J=7.9 Hz), 7.84 (d, 2H, J=7.7 Hz), 7.50 (dd, 1H, J=8.1 and 8.1 Hz), and 4.66 (s, 2H); enol: 13.52 (s, 1H), 10.99 (s, 1H), 8.59 (d, 1H, J=5.5 Hz), 8.25 (s, 1H), 7.65 (d, 1H, J=8.1 Hz), 7.41 (d, 1H, J=5.5 Hz), and 6.50 (s, 1H); shared: 7.58-7.63 (m, 2H), and 7.26 (d, 2H, J=7.9 and 7.9 Hz); ESIMS: 388.05 (M+H⁺).

### Step D: 2,6-Difluoro-N-{3-[(2-{[2-(trifluoroacetyl)-1,2,3,4-tetrahydro-7-isoquinolinyl]amino}-4-pyrimidinyl)acetyl]phenyl}benzamide

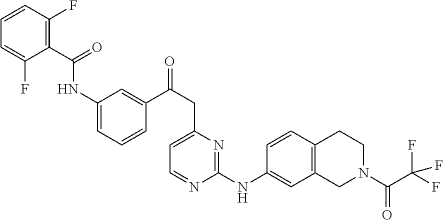

### General Procedure for the Aniline Displacement

To a solution containing 1-4 mL of i-PrOH or trifluoroethanol, optionally with 1,4-dioxane or DMA added as a co-solvent to improve solubility, and 1 equiv of the pyrimidyl chloride is added 1-1.5 equiv of the desired aniline and 1 drop of conc HCl or 4-5 drops of a 4 M solution of HCl in dioxane. The reaction mixture is heated at 70-90° C. for 12-72 h, or heated in a microwave reactor at 150-180° C. for 10-120 min, then allowed to cool to rt. The displacement product is purified by neutralization by the addition of an aqueous solution of NaOH or NaHCO₃, or by the addition of 1-5 equiv of TEA and extracted into an organic solvent such as EtOAc or DCM. The residue from this extraction, or directly from evaporation of solvents from the reaction mixture, is then subjected to silica gel chromatography and/or HPLC purification. In certain instances, precipitation from an organic solvent, or treatment of a solution of the compound with MP-isocyante is utilized to remove excess aniline or other lingering impurities.

### Specific procedure for 2,6-Difluoro-N-{3-[(2-{[2-(trifluoroacetyl)-1,2,3,4-tetrahydro-7-isoquinolinyl]amino}-4-pyrimidinyl)acetyl]phenyl}benzamide

To a solution containing 500 mg (1.29 mmol) of N-{3-[(2-chloro-4-pyrimidinyl)acetyl]phenyl}-2,6-difluorobenzamide, 1.5 mL of i-PrOH, and 0.5 mL of DMA was added 360 mg (1.29 mmol) of 2-(trifluoroacetyl)-1,2,3,4-tetrahydro-7-isoquinolinamine and 1 drop of conc HCl. The reaction mixture was heated at 70° C. for 15 h, then diluted with water, neutralized by the addition of aqueous NaHCO₃, and extracted with DCM. The combined organic layers were dried over MgSO₄, filtered, and concentrated under reduced pressure. The residue was subjected to silica gel chromatography, eluting with a mixture of hexane/EtOAc to give 350 mg (45%) of the title compound of Step D as an orange foam, a mixture of keto and enol tautomers: ESIMS: 596.22 (M+H⁺).

### Step E: 2,6-Difluoro-N-{3-[2-{[2-(4-morpholinyl)ethyl]amino}-5-(2-{[2-(trifluoroacetyl)-1,2,3,4-tetrahydro-7-isoquinolinyl]amino}-4-pyrimidinyl)-1,3-thiazol-4-yl]phenyl}benzamide

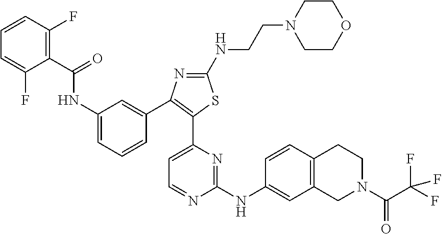

To a solution containing 75 mg (0.13 mmol) of 2,6-difluoro-N-{3-[(2-{[2-(trifluoroacetyl)-1,2,3,4-tetrahydro-7-isoquinolinyl]amino}-4-pyrimidinyl)acetyl]phenyl}benzamide and 1 mL of HOAc was added 6.5 μL (0.13 mmol) of bromine. The reaction mixture was allowed to stir at rt for 15 min, then diluted in water, quenched by the addition of 10% aqueous NaOH, and extracted with DCM. The combined organic layers were dried over MgSO₄ and filtered, and the solvent was removed under reduced pressure. The residue was taken up in 5 mL of dioxane and 26 mg (0.14 mmol) of N-[2-(4-morpholinyl)ethyl]thiourea and 12 mg (0.14 mmol) of MgCO₃ was added. The reaction mixture was heated at 50° C. for 12 h, then cooled to rt, diluted in water, and extracted with DCM. The combined organic layers were dried over MgSO₄ and filtered, and the solvent was removed under reduced pressure. The residue was subjected to silica gel chromatography, eluting with a MeOH/DCM mixture to give 46 mg (48%) of the title compound of Step E as a yellow solid: ¹H-NMR (d⁶-DMSO, 400 MHz) δ 10.88 (s, 1H), 9.48 (s, 1H), 8.15-8.19 (m, 1H), 8.08 (d, 1H, J=5.5 Hz), 7.85 (s, 1H), 7.70-7.74 (m, 2H), 7.64 (s, 1H), 7.54-7.58 (m, 1H), 7.48 (d, 1H, J=9.9 Hz), 7.42 (dd, 1H, J=7.8 Hz), 7.21-7.26 (m, 2H), 7.05-7.09 (m, 1H), 6.31 (d, 1H, J=5.5 Hz), 4.71 (s, 2H), 3.7-3.80 (m, 2H), 3.53-3.55 (m, 4H), 3.38 (t, 2H, J=6.0 Hz), 2.80-2.85 (m, 2H), 2.51 (t, 2H, J=6.4 Hz), and 2.39 (brs, 4H); ESIMS: 765.30 (M+H⁺).

### Step F: 2,6-Difluoro-N-(3-{2-{[2-(4-morpholinyl)ethyl]amino}-5-[2-(1,2,3,4-tetrahydro-7-isoquinolinylamino)-4-pyrimidinyl]-1,3-thiazol-4-yl}phenyl)benzamide

To a solution containing 46 mg (0.06 mmol) of 2,6-difluoro-N-{3-[2-{[2-(4-morpholinyl)ethyl]amino}-5-(2-{[2-(trifluoroacetyl)-1,2,3,4-tetrahydro-7-isoquinolinyl]amino}-4-pyrimidinyl)-1,3-thiazol-4-yl]phenyl}benzamide and 1 mL of THF was added 3 mg (0.12 mmol) of LiOH and 1 mL of water. The reaction mixture was heated at 50° C. for 1 h, then cooled to rt, diluted in water, and extracted with DCM. The combined organic layers were dried over MgSO₄ and filtered and the solvent was removed under reduced pressure to give 29 mg (73%) of the title compound of Example 1 as a yellow solid: ¹H-NMR (d⁶-DMSO, 400 MHz) δ 10.87 (s, 1H), 9.31 (s, 1H), 8.19 (dd, 1H, J=1.8 and 1.8 Hz), 8.05 (s, 1H, J=5.5 Hz), 7.83 (s, 1H), 7.74 (d, 1H, J=8.2 Hz), 7.53-7.57 (m, 1H), 7.52 (s, 1H), 7.42 (t, 1H, J=7.9 Hz), 7.34 (dd, 1H, J=8.2 and 1.8 Hz), 7.20-7.24 (m, 3H), 6.91 (d, 1H, J=8.4 Hz), 6.27 (d, 1H, J=5.5 Hz), 3.83 (s, 2H), 3.54-3.56 (m, 4H), 3.37-3.40 (m, 2H), 2.92 (t, 2H, J=5.7 Hz), 2.60 (t, 2H, J=5.7 Hz), 2.52 (t, 2H, J=6.6 Hz), and 2.40 (brs, 4H); HRMS calcd for C₃₅H₃₄F₂N₈O₂S: 668.2493. Found: 669.2572 (M+H⁺).

### Example 2

### N-{3-[2-Amino-5-(2-{[3-{[2-(dimethylamino)ethyl]oxy}-4-(methyloxy)phenyl]amino}-4-pyrimidinyl)-1,3-thiazol-4-yl]phenyl}-2,6-difluoro-N-methylbenzamide

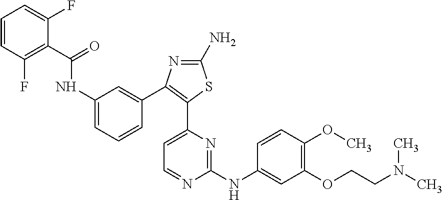

### Step A: N-{3-[2-Amino-5-(2-chloro-4-pyrimidinyl)-1,3-thiazol-4-yl]phenyl}-2,6-difluoro-N-methylbenzamide

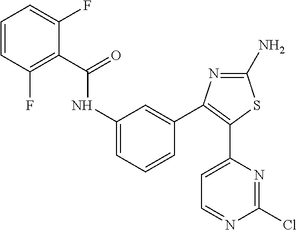

To a slurry containing 1.7 g (4.38 mmol) of N-{3-[(2-chloro-4-pyrimidinyl)acetyl]phenyl}-2,6-difluoro-N-methylbenzamide prepared by a procedure analogous to Example 1, Step C, and 5 mL of DCM was added 0.78 g (4.38 mmol) of NBS. The reaction mixture was allowed to stir for 10 min and the solvent was removed under reduced pressure. The residue was taken up in dioxane and 0.37 g (4.82 mmol) of thiourea and 0.40 g (4.82 mmol) of Mg₂CO₃ was added. The reaction mixture was allowed to stir for 4 h at rt, then heated at 40° C. for an additional 2 h. The reaction mixture was poured into water and EtOAc and 10% aqueous HCl were added. The mixture was filtered to give 1.87 g (96%) of the title compound of Step A as a light yellow solid: ¹H-NMR (400 MHz, DMSO-D6) δ 10.92 (brs, 1H), 8.29 (d, J=5.7 Hz, 1H), 7.95 (brs, 2H), 7.89 (s, 1H), 7.75 (d, J=8.1 Hz, 1H), 7.59 (m, 1H), 7.45 (t, J=8.1 Hz, 1H), 7.26 (m, 3H), and 6.90 (d, J=5.5 Hz, 1H); ESIMS: 444.34 (M+H⁺).

### Step B: N-{3-[2-Amino-5-(2-{[3-{[2-(dimethylamino)ethyl]oxy}-4-(methyloxy)phenyl]amino}-4-pyrimidinyl)-1,3-thiazol-4-yl]phenyl}-2,6-difluoro-N-methylbenzamide

To a slurry containing 232 mg (0.52 mmol) of N-{3-[2-amino-5-(2-chloro-4-pyrimidinyl)-1,3-thiazol-4-yl]phenyl}-2,6-difluoro-N-methylbenzamide and 2 mL of i-PrOH was added 110 mg (0.52 mmol) of 3-{[2-(dimethylamino)ethyl]oxy}-4-(methyloxy) and 1 drop of conc HCl. The reaction mixture was heated at 80° C. for 24 h, then allowed to cool to rt. The crude product was triturated from ether, then taken up in EtOAc and neutralized by the addition of TEA. The solvents were removed under reduced pressure and the residue was subjected to silica gel chromatography, eluting with an EtOAc/MeOH/NH₄OH mixture, to give a yellow powder that was further purified by precipitation from an EtOAc/ether mixture, affording 101 mg (31%) of the title compound of Example 2: ¹H-NMR (d⁶-DMSO, 400 MHz) δ 10.87 (s, 1H), 9.25 (s, 1H), 8.06 (d, 1H, J=5.4 Hz), 7.88 (s, 1H), 7.71 (d, 1H, J=9.0 Hz), 7.54-7.59 (m, 3H), 7.40-7.44 (m, 2H), 7.22-7.26 (m, 4H), 8.85 (d, 1H, J=8.8 Hz), 6.31 (d, 1H, J=5.4 Hz), 4.08 (t, 2H, J=6.9 Hz), 3.71 (s, 3H), 2.71-2.79 (m, 2H), and 2.31 (brs, 6H); HRMS calcd for C₃₁H₂₉F₂N₇O₃S: 617.6781. Found: 618.2093 (M+H⁺).

### Example 3

### N-[3-(2-Amino-5-{2-[3-fluoro-4-{[2-(methyloxy)ethyl]oxy}phenyl)-amino]-4-pyrimidinyl}-1,3-thiazol-4-yl)phenyl]-2,6-difluorobenzamide

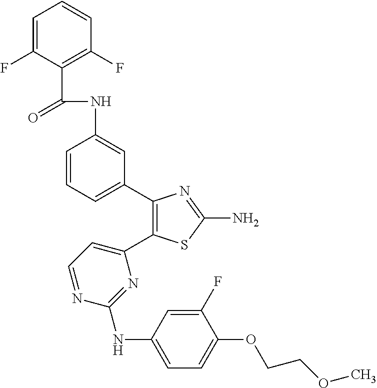

### Step A: 2,6-Difluoro-N-[3-({2-[(3-fluoro-4-{[2-(methyloxy)ethyl]oxy}phenyl)-amino]-4-pyrimidinyl}acetyl)phenyl]benzamide

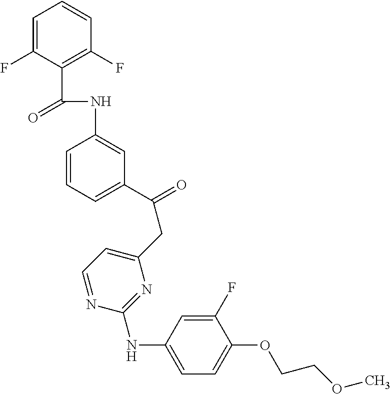

The title compound of Step A was prepared from N-{3-[(2-chloro-4-pyrimidinyl)acetyl]phenyl}-2,6-difluorobenzamide (0.5 g, 1.29 mmol), prepared by a procedure analogous to Example 1, Step C, and (3-fluoro-4-{[2-(methyloxy)ethyl]oxy}phenyl)amine hydrochloride (0.286 g, 1.29 mmol), by a procedure analogous to Example 1, Step D, Yield 0.613 g (85%). MS (ESI) 0/z 559 (M+H)⁺.

### Step B: N-[3-(2-amino-5-{2-[(3-fluoro-4-{[2-(methyloxy)ethyl]oxy}phenyl)-amino]-4-pyrimidinyl}-1,3-thiazol-4-yl)phenyl]-2,6-difluorobenzamide

The title compound of Example 3 was prepared from 2,6-difluoro-N-[3-({2-[(3-fluoro-4-{[2-(methyloxy)ethyl]oxy}phenyl)amino]-4-pyrimidinyl}acetyl)phenyl]-benzamide (0.1 g, 0.18 mmol), Br₂ (9 μL, 0.18 mmol), HOAc (0.3 ml), thiourea (0.012 g, 0.18 mmol), MgCO₃ N-hydrate (27 mg) and 1,4 dioxane (0.5 ml) by a procedure analogous to Example 1, Step E. Purification using silica gel chromatography was followed by trituration from an EtOAc/ether mixture to yield 58 mg (55%). ¹H-NMR (400 MHz, DMSO-d₆) δ 10.87 (s, 1H), 9.51 (s, 1H), 8.09 (d, J=5.7 Hz, 1H), 7.87 (s, 1H), 7.80-7.70 (m, 2H), 7.66 (s, 2H), 7.58 (m, 1H), 7.42 (t, 8.0 Hz, 1H), 7.35 (d, J=9.0 Hz, 1H), 7.28-7.20 (m, 3H), 7.05 (t, J=9.2 Hz, 1H), 6.34 (d, J=5.3 Hz, 1H), 4.10 (t, J=4.6 Hz, 2H), 3.64 (t, J=4.7 Hz, 2H), and 3.30 (s, 3H); MS (ESI) m/z 593 (M+H)⁺.

### Example 4

### N-[3-(5-{2-[(3-Chloro-4-{[2-(dimethylamino)ethyl]oxy}phenyl)-amino]-4-pyrimidinyl}-2-{[3-(4-morpholinyl)propyl]amino}-1,3-thiazol-44)phenyl]-2,6-difluorobenzamide

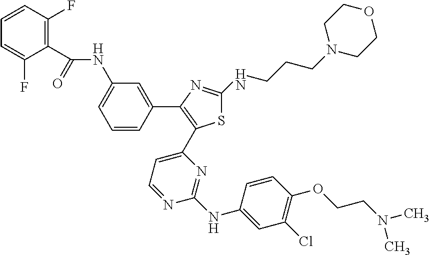

### Step A: N-(3-Chloro-4-{[2-(dimethylamino)ethyl]oxy}phenyl)-4-methyl-2-pyrimidinamine

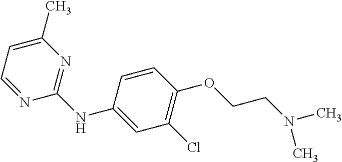

The title compound of Step A was prepared from 2-chloro-4-methylpyrimidine (0.5 g, 3.9 mmol) and {2-[(4-amino-2-chlorophenyl)oxy]ethyl}dimethylamine dihydrochloride (0.97 g, 3.9 mmol) by a procedure analogous to Example 1, Step D. Yield 832 mg (68%)

### Step B: N-[3-({2-[(3-Chloro-4-{[2-(dimethylamino)ethyl]oxy}phenyl)amino]-4-pyrimidinyl}acetyl)phenyl]-2,6-difluorobenzamide

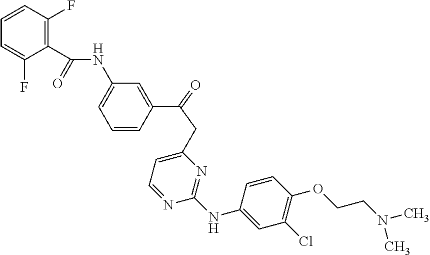

The title compound of Step B was prepared from ethyl 3-{[(2,6-difluorophenyl)carbonyl]amino}benzoate (0.83 g, 2.71 mmol), N-(3-chloro-4-{[2-(dimethylamino)ethyl]oxy}phenyl)-4-methyl-2-pyrimidinamine (0.83 g, 2.71 mmol) and 1 M LiHMDS in THF (10.8 ml, 10.8 mmol) by a procedure analogous to Example 1, Step C. Yield 340 mg (22%). MS (APCI) m/z 567 (M+H)⁺.

### Step C: N-[3-(5-{2-[(3-Chloro-4-{[2-(dimethylamino)ethyl]oxy}phenyl)amino]-4-pyrimidinyl}-2-{[3-(4-morpholinyl)propyl]amino}-1,3-thiazol-4-yl)phenyl]-2,6-difluorobenzamide

The title compound of Example 4 was prepared from N-[3-({2-[(3-chloro-4-{[2-(dimethylamino)ethyl]oxy}phenyl)amino]-4-pyrimidinyl}acetyl)phenyl]-2,6-difluorobenzamide (0.16 g, 0.28 mmol), NBS (0.045 g, 0.25 mmol) and N-[3-(4-morpholinyl)propyl]thiourea (0.057 g, 0.28 mmol) by a procedure analogous to Example 2, Step A. Yield 55 mg (26%)

¹H-NMR (400 MHz, DMSO-d₆) δ 10.90 (s, 1H), 9.50 (s, 1H), 8.32 (t, J=5.3 Hz, 1H), 8.08 (d, J=5.4 Hz, 1H), 8.04 (s, 1H), 7.86 (s, 1H), 7.74 (d, J=8.1 Hz, 1H), 7.57 (m, 1H), 7.50 (dd, J=9.1, 2.1 Hz, 1H), 7.43 (t, J=7.8 Hz, 1H), 7.28-7.20 (m, 3H), 7.07 (d, J=9.0 Hz, 1H), 6.31 (d, J=5.6 Hz, 1H), 4.09 (t, J=5.6 Hz, 2H), 3.54 (m, 4H), 3.29 (m, 2H), 2.72 (m, 2H), 2.34 (m, 6H), 2.29 (s, 6H), and 1.74 (m, 2H);

HRMS C₃₇H₄₀N₈O₃F₂SCl (M+H)⁺ calcd 749.2601. found 749.2612.

### Example 5

### N-(3-{2-Amino-5-[2-({3-chloro-4-[2-(dimethylamino)ethoxy]-phenyl}amino)pyrimidin-4-yl]-1,3-thiazol-4-yl}phenyl)-2,6-difluorobenzamide-formic acid

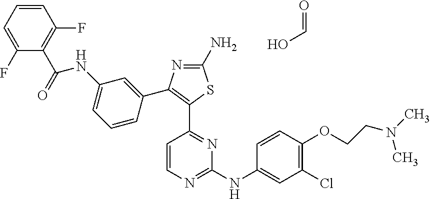

### Step A: N-[3-({2-[(3-Chloro-4-{[2-(dimethylamino)ethyl]oxy}phenyl)amino]-4-pyrimidinyl}acetyl)phenyl]-2,6-difluorobenzamide dihydrochloride

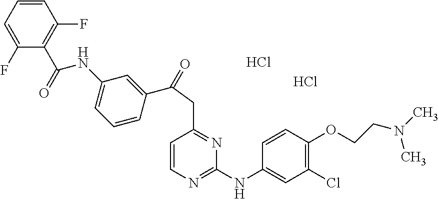

The title compound of Step A was prepared from N-{3-[(2-chloro-4-pyrimidinyl)acetyl]phenyl}-2,6-difluorobenzamide (0.5 g, 1.29 mmol) prepared by a procedure analogous to Example 1, Step C, and {2-[(4-amino-2-chlorophenyl)oxy]ethyl}dimethylamine dihydrochloride (0.37 g, 1.29 mmol) by a procedure analogous to Example 1, Step D. Yield 0.62 g (75%) MS (ESI) m/z 566 (M+H)⁺.

### Step B: N-(3-{2-Amino-5-[2-({3-chloro-4-[2-(dimethylamino)ethoxy]phenyl}-amino)pyrimidin-4-yl]-1,3-thiazol-4-yl}phenyl)-2,6-difluorobenzamide-formic acid

N-[3-({2-[(3-chloro-4-{[2-(dimethylamino)ethyl]oxy}phenyl)amino]-4-pyrimidinyl}acetyl)phenyl]-2,6-difluorobenzamide (0.1 g, 1.57 mmol) was placed in a reaction vessel. Acetic acid (0.3 mL) was added followed by the drop wise addition of Br₂ (8.4 μL, 1.57 mmol). The reaction was allowed to stir at room temperature for 3 h. This was followed by an organic/aqueous extraction with EtOAc and saturated sodium bicarbonate solution which, after concentrating the organics, yielded the α-bromoketone intermediate, which was then dissolved in 1,4-dioxane (0.5 mL). Thiourea (0.011 g, 1.72 mmol) and MgCO₃ n-hydrate (0.012 g) were added and the resulting mixture was heated to 90° C. for several hours until by LC/MS it was complete. The reaction was then purified by HPLC to yield 50 mg (50%) of the title compound of Example 5 as a formic acid salt. ¹H-NMR (400 MHz, DMSO-d₆) δ 10.88 (s, 1H), 9.48 (s, 1H), 8.09 (d, J=5.5 Hz, 1H), 7.93 (d, J=2.7 Hz, 1H), 7.87 (s, 1 H), 7.72 (d, J=7.8 Hz, 1H), 7.65 (s, 2H), 7.62-7.53 (m, 2H), 7.41 (t, J=8.0 Hz, 1H), 7.28-7.20 (m, 3H), 7.06 (d, J=8.9 Hz, 1H), 6.33 (d, J=5.6 Hz, 1H), 4.07 (t, J=5.8 Hz, 2H), 2.64 (t, J=5.8 Hz, 2H), and 2.23 (s, 6H); HRMS C₃₀H₂₇ClF₂N₇O₂S (M+H)⁺ calcd 622.1598. found 622.1592.

### Example 6

### 2,6-Difluoro-N-methyl-N-(3-{5-[2-({4-[(methylsulfonyl)methyl]phenyl}amino)-4-pyrimidinyl]-1,3-thiazol-4-yl}phenyl)benzamide

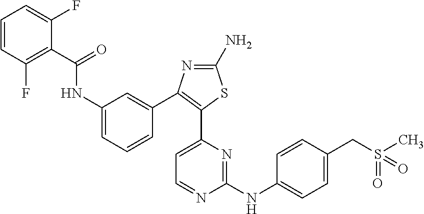

To a slurry containing 77 mg (0.18 mmol) of N-{3-[5-(2-chloro-4-pyrimidinyl)-1,3-thiazol-4-yl]phenyl}-2,6-difluoro-N-methylbenzamide, prepared by a procedure analogous to Example 2, Step A, and 2 mL of i-PrOH was added 33 mg (0.18 mmol) of 4-[(methylsulfonyl)methyl]aniline, and 1 drop of conc HCl. The reaction mixture was heated at 90° C. for 48 h and allowed to cool to rt, and the solvent was removed under reduced pressure. The residue was neutralized by the addition of aqueous NaHCO₃ and extracted with DCM. The combined organic layers were dried over MgSO₄ and filtered, and the solvents were removed under reduced pressure. The residue was subjected to silica gel chromatography to give 50 mg (48%) of the title compound of Example 6 as an orange solid: ¹H-NMR (d⁶-DMSO, 400 MHz) δ 10.89 (s, 1H), 9.86 (s, 1H), 8.36 (d, 1H, J=5.2 Hz), 7.93 (s, 1H), 7.75-7.78 (m, 1H), 7.73 (d, 2H, J=8.6 Hz), 7.52-7.60 (m, 1H), 7.44 (t, 1H, J=7.9 Hz), 7.29-7.32 (m, 4H), 7.27 (t, 2H, J=8.6 Hz), 6.63 (d, 1H, J=5.2 Hz), and 4.36 (s, 2H), 2.76 (s, 3H); HRMS calcd for C₂₈H₂₁F₂N₅O₃S₂: 577.1054. Found: 578.1124 (M+H⁺).

### Example 7

### N-[3-(2-Amino-5-{2-[(3-fluorophenyl)amino]-4-pyrimidinyl}-1,3-thiazol-4-yl)phenyl]-2,6-difluoro-N-methylbenzamide

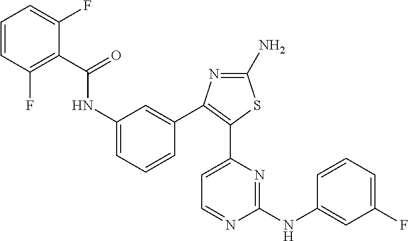

To a slurry containing 100 mg (0.23 mmol) of N-{3-[2-amino-5-(2-chloro-4-pyrimidinyl)-1,3-thiazol-4-yl]phenyl}-2,6-difluoro-N-methylbenzamide, prepared by a procedure analogous to Example 2, Step A, and 2 mL of i-PrOH was added 24 μL (0.25 mmol) of 3-fluorophenyl amine and 2 drops of conc HCl. The reaction mixture was heated at 90° C. for 12 h, then allowed to cool to rt and neutralized by the addition of 0.1 mL of triethyl amine. The solvents were removed under reduced pressure and the residue was purified by HPLC to give 60 mg (51%) of the title compound of Example 7 as a yellow solid: ¹H-NMR (d⁶-DMSO, 400 MHz) δ 10.87 (s, 1H), 9.73 (s, 1H), 8.13 (d, 1H, J=5.5 Hz), 7.89 (s, 1H), 7.81 (d, 1H, J=12.6 Hz), 7.73 (d, 1H, J=8.2 Hz), 7.69 (s, 2H), 7.55-7.61 (m, 1H), 7.56 (t, 2H, J=7.1 Hz), 7.22-7.29 (m, 4H), 6.72 (t, 1H, J=7.9 Hz), and 6.40 (d, 1H, J=5.5 Hz); HRMS cald for C₂₆H₁₇F₃N₆OS: 518.1137. Found: 519.1215 (M+H⁺).

### Example 8

### N-(3-{2-Amino-5-[2-({3-[2-(aminosulfonyl)ethyl]phenyl}amino)-4-pyrimidinyl]-1,3-thiazol-4-yl}phenyl)-2,6-difluoro-N-methylbenzamide

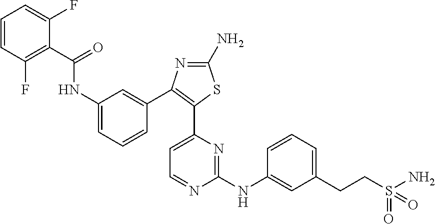

### Step A: 2-(3-Aminophenyl)ethanesulfonamide

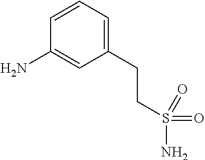

To a solution containing 4.8 g (20.8 mmol) of 2-(3-nitrophenyl)ethanesulfonamide and 75 mL of EtOH was added 400 mg of platinum on carbon. The mixture was subjected to an atmosphere of hydrogen gas at 40 psi for 13 h, then filtered. The solvents were removed under reduced pressure to give 900 mg (24%) of 2-(3-aminophenyl)ethanesulfonamide as an off-white solid: ¹H-NMR (400 MHz, DMSO-d⁶) δ 6.91 (dd, J=8.6 and 7.3 Hz, 1H), 6.81 (s, 2H), 6.33-6.37 (m, 3H), 4.98 (s, 2H), 3.10-3.14 (m, 2H), and 2.77-2.79 (m, 2H); ESIMS: 200.11.

### Step B: N-(3-{2-amino-5-[2-({3-[2-(aminosulfonyl)ethyl]phenyl}amino)-4-pyrimidinyl]-1,3-thiazol-4-yl}phenyl)-2,6-difluoro-N-methylbenzamide

To a slurry containing 100 mg (0.23 mmol) of N-{3-[2-amino-5-(2-chloro-4-pyrimidinyl)-1,3-thiazol-4-yl]phenyl}-2,6-difluoro-N-methylbenzamide, prepared by a procedure analogous to Example 2, Step A, and 2 mL of i-PrOH was added 45 mg (0.23 mmol) of 2-(3-aminophenyl)ethanesulfonamide and 2 drops of conc HCl. The reaction mixture was heated at 90° C. for 12 h, then allowed to cool to rt and neutralized by the addition of 0.1 mL of triethyl amine. The solvents were removed under reduced pressure and the residue was purified by silica gel chromatography, eluting with a DCM/EtOAc mixture, to give 40 mg (34%) of the title compound of Example 8 as a yellow solid: H¹—NMR (400 MHz, DMSO-D⁶) δ 10.88 (s, 1H), 9.48 (s, 1H), 8.11 (d, J=5.3 Hz, 1H), 7.89 (s, 1H), 7.73 (d, J=7.5 Hz, 1H), 7.69 (s, 1 H), 7.54-7.61 (m, 4H), 7.43 (t, J=8.0 Hz, 1H), 7.17-7.27 (m, 4H), 6.90 (s, 2H), 6.85 (d, J=6.8 Hz, 1H), 6.36 (d, J=5.3 Hz, 1H), 3.23-3.30 (m, 2H), and 2.96-3.00 (m, 2H); HRMS calcd for C₂₈H₂₃F₂N₇O₃S₂: 607.1272. Found: 608.1350 (M+H⁺).

### Example 9

### N-[3-(2-Amino-5-{2-[(3-oxo-3,4-dihydro-2H-1,4-benzoxaziN-7-yl)amino]-4-pyrimidinyl}-1,3-thiazol-4-yl)phenyl]-2,6-difluoro-N-methylbenzamide hydrochloride

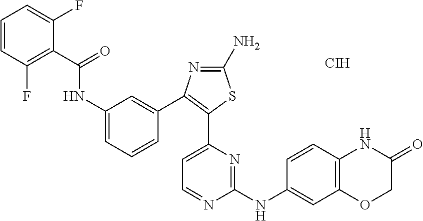

To a slurry containing 100 mg (0.23 mmol) of N-{3-[2-amino-5-(2-chloro-4-pyrimidinyl)-1,3-thiazol-4-yl]phenyl}-2,6-difluoro-N-methylbenzamide, prepared by a procedure analogous to Example 2, Step A, and 2 mL of i-PrOH was added 41 mg (0.23 mmol) of 7-amino-2H-1,4-benzoxazin-3(4H)-one. The reaction mixture was heated at 180° C. in a microwave synthesizer for 40 min, then further heated at 190° C. for 20 min, and allowed to cool to rt. The title compound was isolated by filtering the crude reaction mixture and washing with additional i-PrOH, to give 55 mg (38%) of the title compound of Example 9 as an orange solid: ¹H-NMR (400 MHz, DMSO-D⁶) δ 10.87 (s, 1H), 10.56 (s, 1H), 9.43 (s, 1H), 8.08 (d, J=5.3 Hz, 1H), 7.87 (s, 1H), 7.73 (d, J=8.6 Hz, 1H), 7.66 (s, 2H), 7.54-7.61 (m, 1H), 7.40-7.44 (m, 2H), 7.34 (dd, J=8.1 and 2.5 Hz, 1H), 7.21-7.26 (m, 3H), 6.77 (d, J=8.6 Hz, 1H), 6.32 (d, J=5.5 Hz, 1H), and 4.53 (s, 2H); HRMS calcd for C₂₈H₁₉F₂N₇O₃S: 571.1238. Found: 572.1310 (M÷H⁺).

### Example 10

### N-[3-(2-Amino-5-{2-[(2-methyl-1,2,3,4-tetrahydro-7-isoquinolinyl)amino]-4-pyrimidinyl}-1,3-thiazol-4-yl)phenyl]-2,6-difluoro-N-methylbenzamide trifluoroacetate

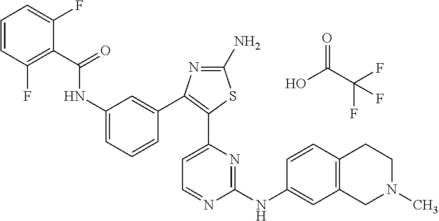

To a slurry containing 100 mg (0.23 mmol) of N-{3-[2-amino-5-(2-chloro-4-pyrimidinyl)-1,3-thiazol-4-yl]phenyl}-2,6-difluoro-N-methylbenzamide, prepared by a procedure analogous to Example 2, Step A, and 2 mL of i-PrOH was added 40 mg (0.23 mmol) of 2-methyl-1,2,3,4-tetrahydro-7-isoquinolinamine. The reaction mixture was heated at 90° C. for 48 h and allowed to cool to rt. The reaction mixture was filtered, and the filtrate was purified on an HPLC to give 96 mg (62%) of the title compound of Example 10 as a yellow solid: ¹H-NMR (400 MHz, DMSO-D⁶) δ 10.89 (s, 1H), 9.88 (s, 1H), 9.59 (s, 1H), 8.12 (d, J=5.5 Hz, 1H), 7.93 (s, 1H), 7.66-7.72 (m, 3H), 7.53-7.59 (m, 3H), 7.43 (t, J=8.0 Hz, 1H), 7.21-7.25 (m, 3H), 7.14 (d, J=9.9 Hz, 1H), 6.38 (d, J=5.3 Hz, 1H), 4.45 (d, J=19.6 Hz, 1H), 4.29 (dd, J=15.4 and 7.9 Hz, 1H), 3.67 (dd, J=15.9 and 4.8 Hz, 1H), 3.29-3.34 (m, 1H), 2.96-3.04 (m, 2H), and 2.95 (d, J=4.8 Hz, 3H); HRMS calcd for C₃₀H₂₅F₂N₇OS: 569.1809. Found: 570.1884 (M+H⁺).

### Example 11

### N-{3-[2-Amino-5-(2-{[4-(methyloxy)-3-(4-methyl-1-piperazinyl)phenyl]amino}-4-pyrimidinyl)-1,3-thiazol-4-yl]phenyl}-2,6-difluorobenzamide trifluoroacetate

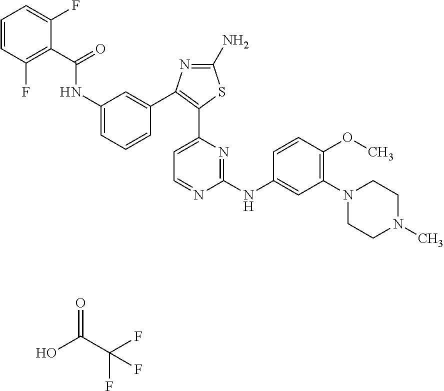

To a slurry containing 100 mg (0.23 mmol) of N-{3-[2-amino-5-(2-chloro-4-pyrimidinyl)-1,3-thiazol-4-yl]phenyl}-2,5-difluoro-N-methylbenzamide, prepared by a procedure analogous to Example 2, Step A, and 5 mL of i-PrOH was added 55 mg (0.23 mmol) of 4-(methyloxy)-3-(4-methyl-1-piperazinyl)phenyl amine and 4 drops of conc HCl. The reaction mixture was heated at 90° C. for 36 h, allowed to cool to rt, and neutralized by the addition of 0.1 mL of TEA. The solvent was removed under reduced pressure and the residue was purified by HPLC to give 21 mg (15%) of the title compound of Example 11 as a yellow solid: ¹H-NMR (d⁶-DMSO, 400 MHz) δ 10.87 (s, 1H), 9.60 (s, 1H), 9.29 (s, 1H), 8.04 (d, 1H, J=5.5 Hz), 7.91 (s, 1H), 7.72 (s, 2H), 7.67 (d, 1H, J=8.7 Hz), 7.55-7.58 (m, 1H), 7.37-7.43 (m, 2H), 7.36 (s, 1H), 7.21-7.25 (m, 3H), 6.89 (d, 1H, J=8.9 Hz), 6.29 (d, 1H, J=5.5 Hz), 3.75 (s, 3H), 3.50 (s, 3H), 3.18-3.22 (m, 2H), and 2.82-2.93 (m, 6H); HRMS calcd for C₃₂H₃₀F₂N₈O₂S: 628.2180. Found: 629.2253 (M+H⁺).

### Example 12

### N-{3-[2-Amino-5-(2-{[2-(dimethylamino)-2,3-dihydro-1H-indeN-5-yl]amino}-4-pyrimidinyl)-1,3-thiazol-4-yl]phenyl}-2,6-difluorobenzamide

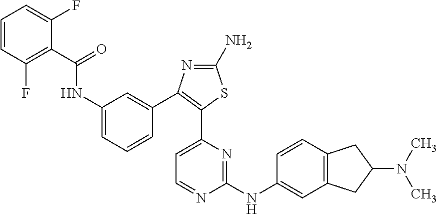

### Step A: N,N-Dimethyl-5-nitro-2,3-dihydro-1H-inden-2-amine

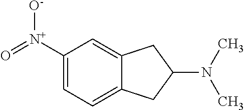

(5-Nitro-2,3-dihydro-1H-inden-2-yl)amine hydrochloride (5.52 g, 26 mmol), paraformaldehyde (4.1 g, 128 mmol), sodiumcyanoborohydride (8.1 g, 128 mmol), and HOAc (7.4 mL, 128 mmol) were combined in 1,2-DCE (200 mL) and the suspension was heated at reflux for 15 h. The reaction was cooled and quenched with saturated aqueous NaHCO₃. The organic layer was separated, washed with brine, dried with MgSO₄, and concentrated to an oil. The crude material was purified by silica gel flash column chromatography using a mixture of DCM, MeOH, and ammonium hydroxide to yield 2.7 g (52%) of a yellow oil that crystallized while stored on the bench top to give N,N-dimethyl-5-nitro-2,3-dihyd-1H-inden-2-amine. ¹H-NMR (400 MHz, CDCl₃) δ 8.07 (m, 2H), 7.36 (d, J=8.1 Hz, 1H), 4.13 (m, 1H), 3.34 (m, 4H), and 2.74 (s, 6H).

### Step B: (5-Amino-2,3-dihydro-1H-inden-2-yl)dimethylamine

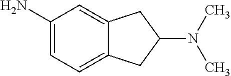

Dimethyl(5-nitro-2,3-dihydro-1H-inden-2-yl)amine (1.5 g, 7.3 mmol)) was dissolved in MeOH and stirred vigorously with of 5% palladium on carbon (250 mg) under a 55 psi atmosphere of hydrogen for 15 h. The reaction was filtered through Celite and the solvent was removed under vacuum. The reaction produced 1.2 g (94%) of the white powder (5-amino-2,3-dihydro-1H-inden-2-yl)dimethylamine). ¹H-NMR (400 MHz, DMSO-d₆) δ 6.84 (d, J=8.2 Hz, 1H), 6.41 (s, 1H), 6.38-6.36 (m, 1H), 4.88 (s, 2H), 3.86 (quint, J=8.8 Hz, 1H), 3.16-2.88 (m, 4H), and 2.60 (s, 6H).

### Step C: N-{3-[2-Amino-5-(2-{[2-(dimethylamino)-2,3-dihydro-1H-indeN-5-yl]amino}-4-pyrimidinyl)-1,3-thiazol-4-yl]phenyl}-2,6-difluorobenzamide

To a slurry containing 100 mg (0.23 mmol) of N-{3-[2-amino-5-(2-chloro-4-pyrimidinyl)-1,3-thiazol-4-yl]phenyl}-2,5-difluoro-N-methylbenzamide, prepared by a procedure analogous to Example 2, Step A, and 5 mL of i-PrOH was added 55 mg (0.23 mmol) of (5-amino-2,3-dihydro-1H-inden-2-yl)dimethylamine and 2 drops of conc HCl. The reaction mixture was heated at 90° C. for 48 h, allowed to cool to rt, and neutralized by the addition of 0.1 mL of TEA. The solvent was removed under reduced pressure and the residue was subjected to silica gel chromatography to give 41 mg (31%) of the title compound of Example 12 as a yellow solid: ¹H-NMR (d⁶-DMSO, 400 MHz) δ 10.86 (s, 1H), 9.34 (s, 1H), 8.05 (d, 1H, J=5.5 Hz), 7.86 (s, 1 H), 7.71 (d, 1H, J=8.0 Hz), 7.54-7.61 (m, 4H), 7.38-7.43 (m, 2H), 7.20-7.25 (m, 3 H), 7.03 (d, 1H, J=8.3 Hz), 6.30 (d, 1H, J=5.5 Hz), 2.89-3.01 (m, 3H), 2.62-2.79 (m, 2H), and 2.20 (brs, 6H); HRMS calcd for C₃₁H₂₇F₂N₇OS: 583.1966. Found: 584.2038 (M+H⁺).

### Example 13

### N-{3-[2-Amino-5-(2-{[3-(1-pyrrolidinylmethyl)phenyl]amino}-4-pyrimidinyl)-1,3-thiazol-4-yl]phenyl}-2,6-difluorobenzamide

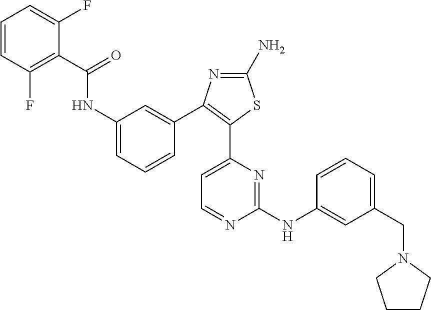

To a slurry containing 100 mg (0.23 mmol) of N-{3-[2-amino-5-(2-chloro-4-pyrimidinyl)-1,3-thiazol-4-yl]phenyl}-2,5-difluoro-N-methylbenzamide, prepared by a procedure analogous to Example 2, Step A, and 5 mL of i-PrOH was added 55 mg (0.23 mmol) of 3-(1-pyrrolidinylmethyl)phenyl amine and 2 drops of conc HCl. The reaction mixture was heated at 90° C. for 48 h, allowed to cool to rt, and neutralized by the addition of 0.1 mL of TEA. The solvent was removed under reduced pressure and the residue was subjected to silica gel chromatography to give 57 mg (31%) of the title compound of Example 12 as a yellow solid: ¹H-NMR (d⁶-DMSO, 400 MHz) δ 10.87 (s, 1H), 9.43 (s, 1H), 8.09 (d, 1H, J=5.3 Hz), 7.88 (s, 1H), 7.72 (d, 1H, J=8.0 Hz), 7.67 (d, 1H, J=8.6 Hz), 7.63 (s, 2H), 7.56-7.62 (m, 1H), 7.42 (t, 1H, J=8.1 Hz), 7.24 (t, 2H, J=8.1 Hz), 7.17 (t, 1H, J=7.6 Hz), 6.87 (d, 1H, J=7.5 Hz), 6.33 (d, 1H, J=5.3 Hz), 3.53 (s, 2H), 2.42 (brs, 2H), and 1.67 (brs, 2H); HRMS calcd for C₃₁H₂₇F₂N₇OS: 583.1966. Found: 584.2048 (M+H⁺).

### Example 14

### N-{3-[2-Amino-5-(2-{[3-(1,3-oxazol-5-yl)phenyl]amino}-4-pyrimidinyl)-1,3-thiazol-4-yl]phenyl}-2,6-difluorobenzamide

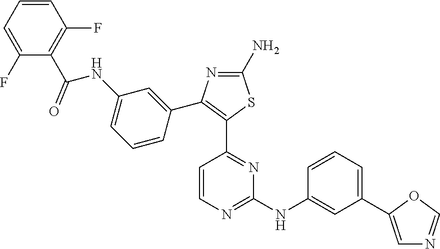

The title compound of Example 14 was prepared from N-{3-[2-amino-5-(2-chloro-4-pyrimidinyl)-1,3-thiazol-4-yl]phenyl}-2,6-difluorobenzamide (0.100 g, 0.22 mmol), prepared by a procedure analogous to Example 2, Step A, and 3-(1,3-oxazol-5-yl)phenyl amine (0.036 g, 0.22 mmol) by a procedure analogous to Example 1, Step D. Yield 27 mg (17%). ¹H-NMR (400 MHz, DMSO-d₆) δ 10.91 (s, 1H), 9.67 (s, 1H), 8.48 (d, J=0.3 Hz, 1H), 8.21 (s, 1H), 8.14 (d, J=5.6 Hz, 1H), 7.90 (s, 1H), 7.73 (t, J=6.8 Hz, 2H), 7.68 (s, 2H), 7.62-7.54 (m, 2H), 7.43 (t, J=7.6 Hz, 1H), 7.38-7.21 (m, 5H), and 6.39 (d, J=5.6 Hz, 1H). MS (ESI) m/z 568 (M+H)⁺

### Example 15

### N-(3-{2-Amino-5-[2-({2-[(methylsulfonyl)methyl]phenyl}amino)pyrimidin-4-yl]-1,3-thiazol-4-yl}phenyl)-2,6-difluorobenzamide

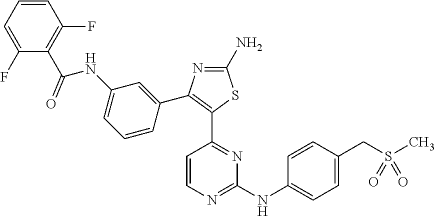

The title compound of Example 15 was prepared from N-{3-[2-amino-5-(2-chloro-4-pyrimidinyl)-1,3-thiazol-4-yl]phenyl}-2,6-difluorobenzamide, prepared by a procedure analogous to Example 2, Step A, (0.100 g, 0.22 mmol) and 4-[(methylsulfonyl)methyl]-phenyl amine (0.042 g, 0.22 mmol), by a procedure analogous to Example 1, Step D. purifying by silica gel chromatography to yield 50 mg (45%). ¹H-NMR (300 MHz, DMSO-d₆) δ 10.88 (s, 1H), 9.62 (s, 1H), 8.12 (d, J=5.4 Hz, 1H), 7.88 (s, 1H), 7.82-7.71 (m, 3H), 7.67 (s, 2H), 7.58 (m, 1H), 7.43 (t, J=7.9 Hz, 1H), 7.31-7.20 (m, 5H), 6.38 (d, J=5.4 Hz, 1H), 4.39 (s, 2H), and 2.86 (s, 3H); HRMS C₂₈H₂₃F₂N₆O₃S₂ (M+H)⁺ calcd 593.1236. found 593.1233.

### Example 16

### N-[3-(2-Amino-5-{2-[(3,4,5-trimethoxyphenyl)amino]pyrimidiN-4-yl}-1,3-thiazol-4-yl)phenyl]-2,6-difluorobenzamide

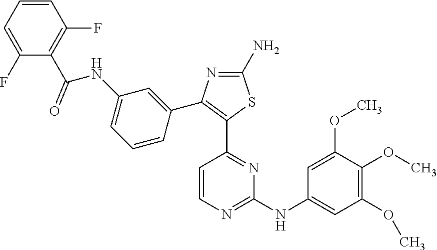

The title compound of Example 16 was prepared from N-{3-[2-amino-5-(2-chloro-4-pyrimidinyl)-1,3-thiazol-4-yl]phenyl}-2,6-difluorobenzamide, prepared by a procedure analogous to Example 2, Step A, (0.075 g, 0.17 mmol) and 3,4,5-tris(methyloxy)phenyl amine (0.031 g, 0.17 mmol) by a procedure analogous to Example 1, Step D, purifying by silica gel chromatography and precipitation from DCM to yield 75 mg (76%). ¹H-NMR (300 MHz, DMSO-d₆) δ 10.89 (s, 1H), 9.34 (s, 1H), 8.09 (d, 5.4 Hz, 1H), 7.89 (s, 1H), 7.73 (d, J=7.8 Hz, 1H), 7.66 (s, 2H), 7.58 (m, 1H), 7.43 (t, J=7.9 Hz, 1H), 7.30-7.20 (m, 3H), 7.17 (s, 2H), 6.34 (d, J=5.3 Hz, 1H), 3.80 (s, 6H), and 3.62 (s, 3H); HRMS C₂₉H₂₆F₂N₆O₄S (M+H)⁺ calcd 591.1621. found 591.1623.

### Example 17

### N-(3-{2-Amino-5-[2-({3-[2-(diethylamino)ethoxy]phenyl}amino)pyrimidiN-4-yl]-1,3-thiazol-4-yl}phenyl)-2,6-difluorobenzamide

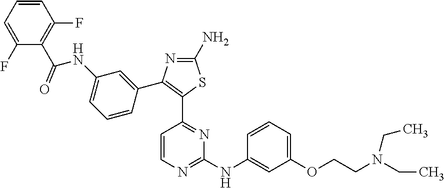

The title compound of Example 17 was prepared from N-{3-[2-amino-5-(2-chloro-4-pyrimidinyl)-1,3-thiazol-4-yl]phenyl}-2,6-difluorobenzamide (0.100 g, 0.22 mmol), prepared by a procedure analogous to Example 2, Step A, and 3-{[2-(diethylamino)ethyl]oxy}phenyl amine (0.047 g, 0.22 mmol), by a procedure analogous to Example 1, Step D, purifying by silica gel chromatography and precipitation from ether to yield 56 mg (40%). ¹H-NMR (300 MHz, DMSO-d₆) δ 10.88 (s, 1H), 9.44 (s, 1H), 8.11 (d, J=5.3 Hz, 1H), 7.88 (s, 1H), 7.73 (d, J=8.1 Hz, 1H), 7.65-7.52 (m, 3H), 7.46-7.39 (m, 2H), 7.34 (d, J=8.5 Hz, 1H), 7.29-7.20 (m, 3H), 7.13 (t, J=8.2 Hz, 1H), 6.50 (d, J=8.2 Hz, 1H), 6.36 (d, J=5.5 Hz, 1H), 4.01 (t, J=6.6 Hz, 2H), 2.77 (t, J=6.4 Hz, 2H), 2.58-2.51 (m, 4H), and 0.96 (t, J=7.1 Hz, 6H); HRMS C₃₂H₃₂F₂N₇O₂S (M+H)⁺ calcd 616.2301. found 616.2301.

### Example 18

### N-[3-(2-Amino-5-{2-[(4-methoxy-3-piperaziN-1-ylphenyl)amino]pyrimidiN-4-yl}-1,3-thiazol-4-yl)phenyl]-2,6-difluorobenzamide

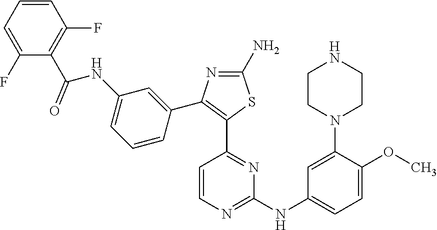

The title compound of Example 18 was prepared from N-{3-[2-amino-5-(2-chloro-4-pyrimidinyl)-1,3-thiazol-4-yl]phenyl}-2,6-difluorobenzamide (0.1 g, 0.22 mmol), prepared by a procedure analogous to Example 2, Step A, and [4-(methyloxy)-3-(1-piperazinyl)phenyl]amine hydrochloride (0.054 g, 0.22 mmol), by a procedure analogous to Example 1, Step D. Yield 53 mg (38%). ¹H-NMR (300 MHz, DMSO-d₆) δ 10.87 (s, 1H), 9.18 (s, 1H), 8.05 (d, J=5.4 Hz, 1H), 7.88 (s, 1H), 7.73 (d, J=8.2 Hz, 1H), 7.64-7.53 (m, 3H), 7.45-7.35 (m, 2H), 7.29-7.20 (m, 4H), 6.80 (d, J=8.8 Hz, 1H), 6.29 (d, J=5.4 Hz, 1H), 3.74 (s, 3H), and 2.92-2.78 (m, 8H); HRMS C₃₁H₂₆F₂N₈O₂S (M+H)⁺ calcd 615.2097. found 615.2097.

### Example 19

### N-{3-[2-Amino-5-(2-{[3-(dimethylamino)-4-(methyloxy)phenyl]amino}-4-pyrimidinyl)-1,3-thiazol-4-yl]phenyl}-2,6-difluorobenzamide

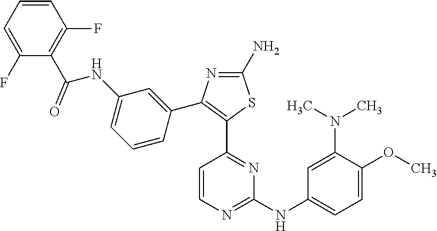

The title compound of Example 19 was prepared from N-{3-[2-amino-5-(2-chloro-4-pyrimidinyl)-1,3-thiazol-4-yl]phenyl}-2,6-difluorobenzamide (0.08 g, 0.18 mmol), prepared by a procedure analogous to Example 2, Step A, and [5-amino-2-(methyloxy)phenyl]dimethylamine (0.03 g, 0.19 mmol) by a procedure analogous to Example 1, Step D. Yield 39 mg (39%). ¹H-NMR (300 MHz, DMSO-d₆) δ 10.87 (s, 1H), 9.16 (s, 1H), 8.05 (d, J=5.3 Hz, 1H), 7.87 (s, 1H), 7.73 (d, J=8.1 Hz, 1H), 7.65-7.53 (m, 3H), 7.42 (t, J=7.9 Hz, 1H), 7.33 (d, J=8.9 Hz, 1H), 7.28-7.20 (m, 4 H), 6.79 (d, J=8.9 Hz, 1H), 6.29 (d, J=5.2 Hz, 1H), 3.74 (s, 3H), and 2.70 (s, GH); HRMS C₂₉H₂₆F₂N₇O₂S (M+H)⁺ calcd 574.1831. found 574.1832.

### Example 20

### N-[3-(2-Amino-5-{2-[(3-oxo-3,4-dihydro-2H-1,4-benzoxaziN-6-yl)amino]-4-pyrimidinyl}-1,3-thiazol-4-yl)phenyl]-2,6-difluorobenzamide

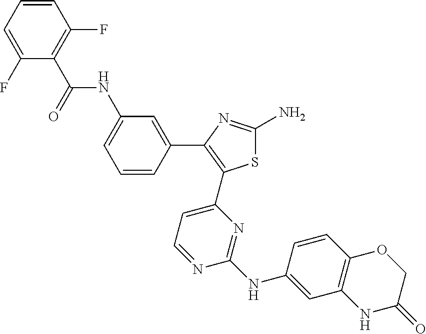

The title compound of Example 20 was prepared from N-{3-[2-amino-5-(2-chloro-4-pyrimidinyl)-1,3-thiazol-4-yl]phenyl}-2,6-difluorobenzamide (0.1 g, 0.22 mmol), prepared by a procedure analogous to Example 2, Step A, and 6-amino-2H-1,4-benzoxaziN-3(4H)-one (0.045 g, 0.27 mmol), by a procedure analogous to Example 1, Step D. Yield 62 mg (52%). ¹H-NMR (400 MHz, DMSO-d₆) δ 10.87 (s, 1H), 10.65 (s, 1H), 9.39 (s, 1H), 8.06 (d, J=5.4 Hz, 1H), 7.87 (s, 1H), 7.73 (d, J=7.9 Hz, 1H), 6.31 (d, J=5.5 Hz, 1H), and 4.49 (s, 2H); HRMS C₂₈H₂₀F₂N₇O₃S (M+H)⁺ calcd 572.1311. found 572.1310.

### Example 21

### N-(3-{2-Amino-5-[2-(1,2,3,4-tetrahydro-7-isoquinolinylamino)-4-pyrimidinyl]-1,3-thiazol-4-yl}phenyl)-2,6-difluorobenzamide

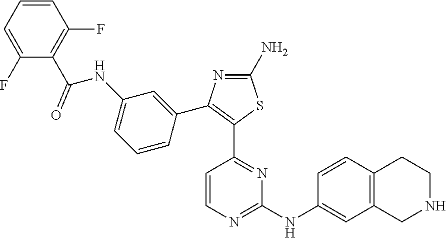

N-{3-[2-Amino-5-(2-{[2-(trifluoroacetyl)-1,2,3,4-tetrahydro-7-isoquinolinyl]amino}-4-pyrimidinyl)-1,3-thiazol-4-yl]phenyl}-2,6-difluorobenzamide was prepared from N-{3-[2-amino-5-(2-chloro-4-pyrimidinyl)-1,3-thiazol-4-yl]phenyl}-2,6-difluorobenzamide (0.1 g, 0.22 mmol), prepared by a procedure analogous to Example 2, Step A, and 2-(trifluoroacetyl)-1,2,3,4-tetrahydro-7-isoquinolinamine hydrochloride (0.063 g, 0.22 mmol), by a procedure analogous to Example 1, Step D. MS (APCI) m/z 652 (M+H)⁺. The material was used directly in the next reaction. To obtain the title compound of Example 21, N-{3-[2-amino-5-(2-{[2-(trifluoroacetyl)-1,2,3,4-tetrahydro-7-isoquinolinyl]amino}-4-pyrimidinyl)-1,3-thiazol-4-yl]phenyl}-2,6-difluorobenzamide directly from above was dissolved in a 3:1 mixture THF:H₂O (4 mL). LiOH (50 mg) was added then the resulting mixture was heated to 50° C. for 30 min. A copious amount of H₂O was added and the resulting solids were filtered and dried to obtain 62 mg (50%) of the title compound of Example 21. ¹H-NMR (400 MHz, DMSO-d₆) δ 10.87 (s, 1H), 9.32 (s, 1H), 8.07 (d, J=5.4 Hz, 1H), 7.87 (s, 1H), 7.73 (d, J=8.2 Hz, 1H), 7.64 (s, 2H), 7.58 (m, 1H), 7.50 (s, 1H), 7.42 (t, J=7.9 Hz, 1H), 7.37 (dd, J=8.3 and 2.0 Hz, 1H), 7.28-7.21 (m, 3H), 6.92 (d, J=8.3 Hz, 1H), 6.30 (d, J=5.8 Hz, 1H), 3.83 (s, 2H), 2.91 (t, J=6.0 Hz, 2H), and 2.60 (t, J=6.0 Hz, 2H); HRMS C₂₉H₂₄F₂N₇OS (M+H)⁺ calcd 556.1726. found 556.1723.

### Example 22

### N-[3-(2-Amino-5-{2-[(3-{[2-(dimethylamino)ethyl]oxy}phenyl) amino]-4-pyrimidinyl}-1,3-thiazol-4-yl)phenyl]-2,6-difluorobenzamide

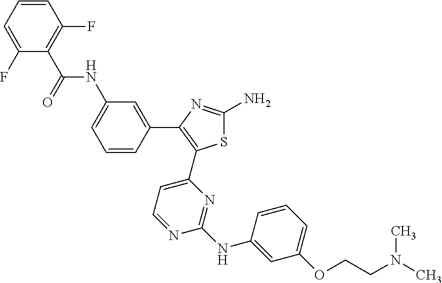

The title compound of Example 22 was prepared from N-{3-[2-amino-5-(2-chloro-4-pyrimidinyl)-1,3-thiazol-4-yl]phenyl}-2,6-difluorobenzamide (0.075 g, 0.17 mmol), prepared by a procedure analogous to Example 2, Step A, and 3-{[2-(dimethylamino)ethyl]oxy}phenyl amine (0.03 g, 0.17 mmol), by a procedure analogous to Example 1, Step D, and purified using HPLC to yield 25 mg (26%) of the title compound of Example 22. ¹H-NMR (400 MHz, DMSO-d₆) δ 10.89 (s, 1H), 9.46 (s, 1H), 8.12 (d, J=5.4 Hz, 1H), 7.89 (s, 1H), 7.74 (d, J=7.9 Hz, 1H), 7.67-7.55 (m, 3H), 7.47-7.40 (m, 2H), 7.36 (d, J=8.4 Hz, 1H), 7.30-7.21 (m, 3H), 7.15 (t, J=8.2 Hz, 1H), 6.52 (dd, J=8.0 and 2.6 Hz, 1H), 6.37 (d, J=5.4 Hz, 1H), 4.06 (t, J=5.9 Hz, 2H), 2.66 (t, J=6.0 Hz, 2H), and 2.24 (s, 6H); HRMS C₃₀H₂₈N₇O₂F₂S (M+H)⁺ calcd 588.1993. found 588.1987.

### Example 23

### 2,6-Difluoro-N-{3-[2-(methylamino)-5-(2-{[3-(1-pyrrolidinylmethyl)phenyl]amino}-4-pyrimidinyl)-1,3-thiazol-4-yl]phenyl}benzamide

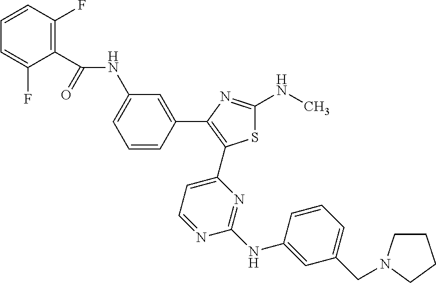

### Step A: N-{3-[5-(2-Chloro-4-pyrimidinyl)-2-(methylamino)-1,3-thiazol-4-yl]phenyl}-2,6-difluorobenzamide

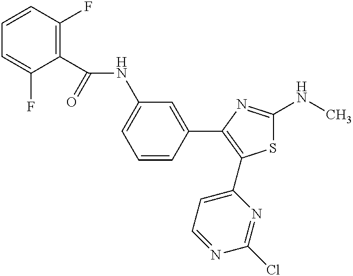

The title compound of Step A was prepared from N-{3-[(E)-2-(2-chloro-4-pyrimidinyl)-1-hydroxyethenyl]phenyl}-2,6-difluorobenzamide (0.4 g, 1.03 mmol), prepared by a procedure analogous to Example 1, Step C, NBS (0.18 g, 1.03 mmol) and N-methylthiourea (0.093 g, 1.03 mmol), by a procedure analogous to Example 2, Step A. Yield 0.171 g (36%). ¹H-NMR (400 MHz, DMSO-d₆) δ 10.92 (s, 1H), 8.50 (m, 1H), 8.28 (d, J=5.8 Hz, 1H), 7.85 (s, 1H), 7.77 (d, J=7.9 Hz, 1H), 7.58 (m, 1H), 7.46 (t, J=7.9 Hz, 1H), 7.30-7.20 (m, 3H), 6.85 (d, J=5.8 Hz, 1H), and 2.89 (d, J=5.0 Hz, 3H); MS (ESI) m/z 458.12 (M+H)⁺.

### Step B: 2,6-Difluoro-N-{3-[2-(methylamino)-5-(2-{[3-(1-pyrrolidinylmethyl)phenyl]amino}-4-pyrimidinyl)-1,3-thiazol-4-yl]phenyl}benzamide

The title compound of Example 23 was prepared from N-{3-[5-(2-chloro-4-pyrimidinyl)-2-(methylamino)-1,3-thiazol-4-yl]phenyl}-2,6-difluorobenzamide (0.083 g, 0.18 mmol) and (1-pyrrolidinylmethyl)phenyl amine (0.032 g, 0.18 mmol), by a procedure analogous to Example 1, Step D. Yield 0.015 g (14%). ¹H-NMR (300 MHz, DMSO-d₆) δ 10.92 (s, 1H), 9.50 (br, 1H), 8.22 (m, 1H), 8.11 (d, J=5.4 Hz, 1H), 7.87 (s, 1H), 7.83-7.74 (m, 2H), 7.66-7.53 (m, 2H), 7.45 (t, J=7.9 Hz, 1H), 7.29-7.19 (m, 4H), 6.94 (br, 1H), 6.32 (d, J=5.4 Hz, 1H), 3.65 (br, 2H), 2.89 (d, J=5.0 Hz, 3H), 2.50 (br, 4H), and 1.73 (br, 4H); MS (ESI) m/z 598.29 (M+H)⁺.

### Example 24

### N-{3-[5-{2-[(3-Chloro-4-{[2-(dimethylamino)ethyl]oxy}phenyl)amino]-4-pyrimidinyl}-2-(methylamino)-1,3-thiazol-4-yl]phenyl}-2,6-difluorobenzamide

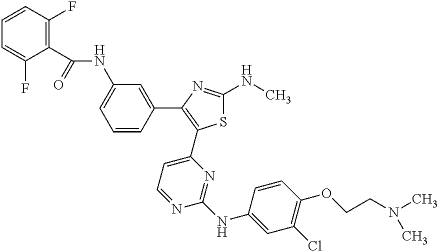

The title compound of Example 24 was prepared from N-{3-[5-(2-chloro-4-pyrimidinyl)-2-(methylamino)-1,3-thiazol-4-yl]phenyl}-2,6-difluorobenzamide (0.088 g, 0.19 mmol), prepared by a procedure analogous to Example 23, Step A, and 3-chloro-4-{[2-(dimethylamino)ethyl]oxy}phenyl amine (0.048 g, 0.19 mmol), by a procedure analogous to Example 1, Step D. Yield 0.04 g (33%). ¹H-NMR (400 MHz, DMSO-d₆) δ 10.90 (s, 1H), 9.48 (s, 1H), 8.21 (d, J=4.9 Hz, 1H), 8.09 (d, J=5.5 Hz, 1H), 8.01 (s, 1H), 7.85 (s, 1H), 7.76 (d, J=8.4 Hz, 1H), 7.58 (m, 1H), 7.51 (d, J=9.1 Hz, 1H), 7.44 (t, J=7.9 Hz, 1H), 7.28-7.20 (m, 3H), 7.06 (d, J=9.2 Hz, 1H), 6.30 (d, J=5.3 Hz, 1H), 4.06 (t, J=5.7 Hz, 2H), 2.88 (d, J=6.2 Hz, 3H), 2.62 (t, J=6.5 Hz, 2H), and 2.22 (s, 6H); MS (ESI) m/z 636.19 (M+H)⁺. HRMS C₃₁H₂₉N₇O₂F₂SCl (M+H)⁺ calcd 636.1760. found 636.1765.

### Example 25

### N-{3-[5-{2-[1,3-Chloro-4-{[2-(dimethylamino)ethyl]oxy}phenyl)amino]-4-pyrimidinyl}-2-(ethylamino)-1,3-thiazol-4-yl]phenyl}-2,6-difluorobenzamide

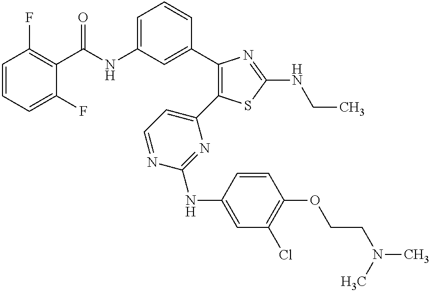

### Step A: N-{3-[5-(2-Chloro-4-pyrimidinyl)-2-(ethylamino)-1,3-thiazol-4-yl]phenyl}-2,6-difluorobenzamide

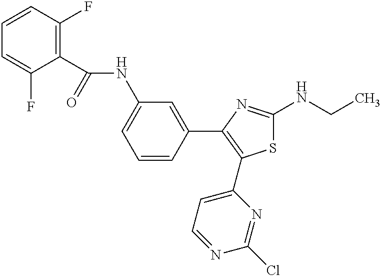

To obtain the desired compound, N-{3-[(E)-2-(2-chloro-4-pyrimidinyl)-1-hydroxyethenyl]phenyl}-2,6-difluorobenzamide (1.22 g, 3.16 mmol), prepared in a procedure analogous to Example 1, Step C, was placed in a round bottom flask with DMF (15 mL). NBS (0.56 g, 3.16 mmol) was added and the resulting mixture was stirred at room temperature for 10 min. N-Ethyl thiourea (0.328 g, 3.47 mmol) was then added and stirring at room temperature was continued for 1 h. EtOAc and H₂O were added to the reaction mixture and the desired was extracted into the organic phase which was then concentrated onto silica gel and purified via column chromatography to yield 0.8 g of the target compound of Step A. Yield 53%. ¹H-NMR (400 MHz, DMSO-d₆) δ ppm 10.90 (s, 1H), 8.55 (t, J=5.4 Hz, 1H), 8.26 (d, J=5.5 Hz, 1H), 7.83 (m, 1H), 7.76 (d, J=8.3, 1H), 7.56 (m, 1H), 7.44 (t, J=7.9 Hz, 1H), 7.19-7.27 (m, 3H), 6.84 (d, J=5.7 Hz, 1H), 3.24-3.32 (m, 2H), and 1.17 (t, J=7.2 Hz, 3H).

### Step B: N-{3-[5-{2-[(3-Chloro-4-{[2-(dimethylamino)ethyl]oxy}phenyl)amino]-4-pyrimidinyl}-2-(ethylamino)-1,3-thiazol-4-yl]phenyl}-2,6-difluorobenzamide

To obtain the title compound of Example 25, N-{3-[5-(2-chloro-4-pyrimidinyl)-2-(ethylamino)-1,3-thiazol-4-yl]phenyl}-2,6-difluorobenzamide (0.1 g, 0.212 mmol) and 3-chloro-4-{[2-(dimethylamino)ethyl]oxy}phenyl amine (0.053 g, 0.212 mmol) were combined with i-PrOH (2 mL) and concentrated HCl (2 drops) in a microwave vial. The reaction was heated to 180° C. for 15 min in the microwave then cooled to rt. TEA (approx. 0.1 mL) and silica gel were combined with the reaction and the resulting mixture was concentrated to dryness. Column chromatography using EtOAc, MeOH and ammonium hydroxide yielded fractions which were concentrated to dryness. The resulting material was then sonicated in ether and the solid that persisted was filtered off to yield 85 mg of the title compound of Example 25 (Yield 62%): ¹H-NMR (400 MHz, DMSO-d₆) δ 10.88 (s, 1H), 9.46 (s, 1H), 8.26 (t, J=5.7 Hz, 1H), 8.07 (d, J=5.6 Hz, 1H), 8.00 (d, J=2.5 Hz, 1H), 7.83 (s, 1H), 7.75 (d, J=7.8 Hz, 1H), 7.56 (m, 1H), 7.49 (dd, J=9.2, 2.5 Hz, 1H), 7.42 (t, J=8.1 Hz, 1H), 7.26-7.19 (m, 3H), 7.05 (d, J=9.1 Hz, 1H), 6.30 (d, J=5.5 Hz, 1H), 4.05 (t, J=5.9 Hz, 2H), 3.26 (m, 2H), 2.62 (t, J=6.3 Hz, 2H), 2.20 (s, 6H), and 1.18 (t, J=7.5 Hz, 3H); HRMS C₃₂H₃₁N₇O₂F₂SCl (M+H)⁺ calcd 650.1917. found 650.1919.

### Example 26

### N-(3-{2-(Ethylamino)-5-[2-({3-fluoro-4-[2-(1-pyrrolidinyl)ethyl]phenyl}amino)-4-pyrimidinyl]-1,3-thiazol-4-yl}phenyl)-2,6-difluorobenzamide trifluoroacetate

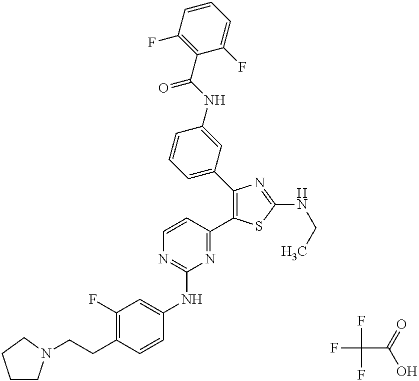

### Step A: Diethyl (2-fluoro-4-nitrophenyl)propanedioate

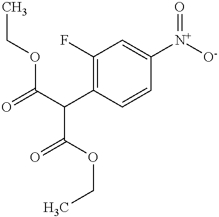

To a suspension of 60% NaH (5.28 g, 131.99 mmol) in dry 100 mL of DMF, diethyl malonate (20.11 g, 125.71 mmol) was added dropwise while cooling to 0° C. After stirring for 10 min at 0° C., a solution of 3,4-difluoronitrobenzene (10.00 g, 62.85 mmol) in 10 mL of DMF was added dropwise. The reaction was stirred overnight while heating to 70° C. and then cooled to RT, quenched with sat. aqueous NH₄Cl, and extracted with EtOAc x4. Combined organics were washed with brine x3 and dried over MgSO₄. Silica gel was added and the solvent was removed under reduced pressure and the residue was purified via flash chromatography with EtOAc/Hex 0-100%. The diester was isolated as a yellow oil (6.5 g, quantitative yield). ES-LCMS m/z 300 (M+H).

### Step B: Ethyl (2-fluoro-4-nitrophenyl)acetate

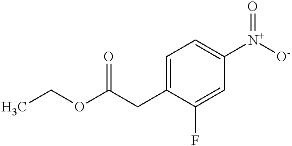

Diethyl (2-fluoro-4-nitrophenyl)propanedioate (1.00 g, 3.34 mmol) was dissolved in 20 mL of DMSO and lithium chloride (0.582 g, 10.03 mmol) was added, followed by water (10 μL). The reaction mixture was heated to 120° C. for 3 h, cooled to RT, diluted with sat. ammonium chloride, and extracted with EtOAc x3. The organic layer was washed with water and brine and dried over MgSO₄. The solvents were removed under reduced pressure and the residue was purified via silica gel chromatography with EtOAC/Hexanes to give 0.458 g, 60% yield, of product. ES-LCMS m/z 228 (M+H).

### Step C: (2-Fluoro-4-nitrophenyl)acetaldehyde

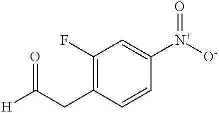

Ethyl (2-fluoro-4-nitrophenyl)acetate (0.458 g, 2.02 mmol) was dissolved in 20 mL of DCM and cooled to −78° C. Dibal-H (1 M in toluene 2.22 mL, 2.22 mmol) was added and stirring at −78° C. was continued for 30 min. The reaction was quenched with potassium tartarate, diluted with 25 mL of DCM, and stirred overnight. The layers were separated and further extracted with DCM x3. The solvents were removed to give 0.369 g, quantitative yield, of desired product. ES-LCMS m/z 184 (M+H).

### Step D: 1-[2-(2-Fluoro-4-nitrophenyl)ethyl]pyrrolidine

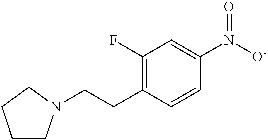

(2-Fluoro-4-nitrophenyl)acetaldehyde (0.369 g, 2.02 mmol) and pyrrolidine (0.143 g, 2.02 mmol) were dissolved in 10 mL of DCM and stirred for 10 min. Sodiumtriacetoxyborohydride (0.851 g, 4.03 mmol) was added and the reaction mixture was stirred overnight at rt. The reaction mixture was quenched with NaHCO₃, diluted with DCM, filtered, and washed with NaHCO₃ x2. The organic layers were dried over MgSO₄, loaded directly onto silica, and purified via flash chromatography 0-10% MeOH/DCM gradient to give 0.270 g, 56% yield, of a brown oil. ES-LCMS m/z 239 (M+H).

### Step E: 3-Fluoro-4-[2-(1-pyrrolidinyl)ethyl]phenyl amine

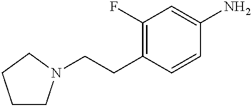

1-[2-(2-Fluoro-4-nitrophenyl)ethyl]pyrrolidine (0.270 g, 1.13 mmol) was dissolved in 15 mL of MeOH and 10% Pd/C (0.025 g) was added. The reaction mixture was stirred under H₂ (60 psi) for 2 h, then filtered through celite and concentrated to yield 0.236 g (quantitative yield) as a light brown oil. ¹H-NMR (400 MHz, CHCl₃-d) δ ppm 6.94 (t, J=8.33 Hz, 1H), 6.31-6.38 (m, 2H), 3.62 (s, 2H), 2.72 (s, 3H), 2.59 (s, 3 H), and 1.77 (s, 4H); ES-LCMS m/z 209 (M+H).

### Step F: N-(3-{2-(Ethylamino)-5-[2-({3-fluoro-4-[2-(1-pyrrolidinyl)ethyl]phenyl}amino)-4-pyrimidinyl]-1,3-thiazol-4-yl}phenyl)-2,6-difluorobenzamide trifluoroacetate

Title compound of Example 26 was synthesized using the standard microwave chloride displacement conditions analogous to Example 1, Step D, in i-PrOH using N-{3-[5-(2-chloro-4-pyrimidinyl)-2-(ethylamino)-1,3-thiazol-4-yl]phenyl}-2,6-difluorobenzamide (0.100 g, 0.21 mmol), prepared by a procedure analogous to Example 25, Step A, and 3-fluoro-4-[2-(1-pyrrolidinyl)ethyl]phenyl amine (0.053 g, 0.25 mmol). The solvent was removed and the residue was taken up in DMSO/MeOH (2:1) and purified via HPLC. Desired fractions were combined and dried to give 0.085 g, 63% yield, of desired product as a solid. ¹H-NMR (400 MHz, DMSO-d₆) δ 10.91 (s, 1H), 9.74 (s, 1H), 9.52-9.60 (m, 1H), 8.34 (t, J=5.5 Hz, 1H), 8.13 (d, J=5.5 Hz, 1H), 7.87-7.90 (m, 1H), 7.85 (dd, J=13.6 and 2.2 Hz, 1H), 7.73 (d, J=7.3 Hz, 1H), 7.58 (t, J=7.7 Hz, 1H), 7.39-7.46 (m, 2H), 7.20-7.27 (m, 3H), 6.37 (d, J=5.5 Hz, 1H), 3.50-3.62 (m, 2H), 3.26-3.37 (m, 4H), 3.05 (dd, J=11.4 and 6.2 Hz, 2H), 2.86-2.96 (m, 2H), 2.01 (t, J=8.3 Hz, 2H), 1.80-1.90 (m, 2H), and 1.20 (t, J=7.2 Hz, 3H); ES-LCMS m/z 644 (M+H).

### Example 27

### N-[3-(5-{2-[(3-Chloro-4-{[2-(1-pyrrolidinyl)ethyl]oxy]phenyl)amino}-4-pyrimidinyl}-2-{[2-(methyloxy)ethyl]amino}-1,3-thiazol-4-yl)phenyl]-2,6-difluorobenzamide

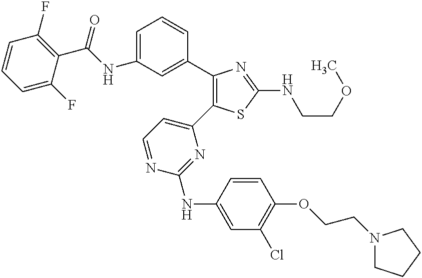

### Step A: 1-{2-[(2-Chloro-4-nitrophenyl)oxy]ethyl}pyrrolidine

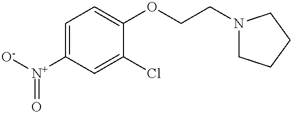

2-Chloro-1-fluoro-4-nitrobenzene (2.5 g, 14.2 mmol), 2-(1-pyrrolidinyl)ethanol (3.3 mL, 28.5 mmol), cesium carbonate (23 g, 71.2 mmol) and DMF (10 mL) were stirred for 15 h at 90° C. The reaction product was partitioned between EtOAc and water. The organic fraction was washed with brine and dried over MgSO₄. The crude oil was purified by silica gel column chromatography (0-100% gradient, (90 CH₂Cl₂: 9 MeOH: 1 NH₄OH—CH₂Cl₂)). Purification yielded 2.68 g (70%) of a yellow oil. MS (ESI): M+H=271.02.

### Step B: 3-Chloro-4-{[2-(1-pyrrolidinyl)ethyl]oxy}phenyl amine hydrochloride

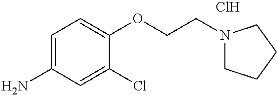

1-{2-[(2-chloro-4-nitrophenyl)oxy]ethyl}pyrrolidine (2.68 g, 9.9 mmol) was combined with 5% platinum on carbon (200 mg) and EtOH (50 mL). The reaction was stirred vigorously for 3 h at rt under a hydrogen balloon. The platinum was removed by filtration through Celite and the crude filtrate was concentrated to an oil under vacuum. The residual crude material was dissolved in MeOH and treated with 6.5 mL (1 equivalent) of hydrochloric acid solution (1 N HCl in Et₂O). The solvent was removed under vacuum providing 1.56 g (57%) of 3-chloro-4-{[2-(1-pyrrolidinyl)ethyl]oxy}phenyl amine hydrochloride, a tan powder. ¹H-NMR (400 MHz, DMSO-d₆) δ 10.90 (brs, 1H), 6.95 (d, 1H), 6.75 (s, 1H), 6.59 (d, 1H), 6.10 (brs, 1H), 4.24 (t, 2H), 3.57 (brs, 2H), 3.51 (t, 2H), 3.09 (brs, 2H), 1.98 (brs, 2H) and 1.85 (brs, 2H).

### Step C: N-[3-(5-(2-Chloro-4-pyrimidinyl)-2-{[2-(methyloxy)ethyl]amino}-1,3-thiazol-4-yl)phenyl]-2,6-difluorobenzamide

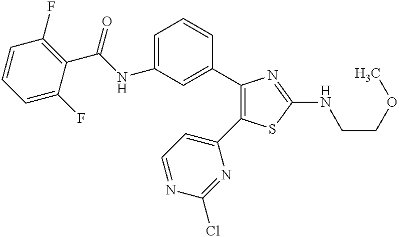

The title compound of Step C was prepared from N-{3-[(E)-2-(2-chloro-4-pyrimidinyl)-1-hydroxyethenyl]phenyl}-2,6-difluorobenzamide (0.4 g, 1.03 mmol), prepared by a procedure analogous to Example 1, Step C, NBS (0.18 g, 1.03 mmol) and N-[2-(methyloxy)ethyl]thiourea (0.138 mg, 1.03 mmol), by a procedure analogous to Example 2, Step A. Yield 0.2 g (38%): ¹H-NMR (400 MHz, DMSO-d₆) δ 10.92 (s, 1 H), 8.62 (m, 1H), 8.28 (d, J=5.6 Hz, 1H), 7.84 (s, 1H), 7.79 (d, J=8.3 Hz, 1H), 7.58 (m, 1H), 7.46 (t, J=8.0 Hz, 1H), 7.29-7.20 (m, 3H), 6.86 (d, J=5.5 Hz, 1H), 3.49 (m, 4H), and 3.27 (s, 3H); MS (ESI) m/z 502.13 (M+H)⁺.

### Step D: N-[3-(5-{2-[(3-Chloro-4-{[2-(1-pyrrolidinyl)ethyl]oxy}phenyl)amino]-4-pyrimidinyl}-2-{[2-(methyloxy)ethyl]amino}-1,3-thiazol-4-yl)phenyl]-2,6-difluorobenzamide

The title compound of Example 27 was prepared from N-[3-(5-(2-chloro-4-pyrimidinyl)-2-{[2-(methyloxy)ethyl]amino}-1,3-thiazol-4-yl)phenyl]-2,6-difluorobenzamide (0.1 g, 0.2 mmol) and 3-chloro-4-{[2-(1-pyrrolidinyl)ethyl]oxy}phenyl amine (0.055 g, 0.2 mmol) by a procedure analogous to Example 1, Step D. Yield 0.05 g (35%). ¹H-NMR (400 MHz, DMSO-d₆) δ 10.90 (s, 1H), 9.49 (s, 1H), 8.36 (m, 1H), 8.10 (d, J=5.3 Hz, 1H), 8.00 (s, 1H), 7.84 (s, 1H), 778 (d. J=7.8 Hz, 1H), 7.58 (m, 1H), 7.53 (d, J=9.1 Hz, 1H), 7.44 (t, J=7.9 Hz, 1H), 7.29-7.21 (m, 3H), 7.06 (d, J=8.9 Hz, 1H), 6.32 (d, J=5.5 Hz, 1H), 4.09 (t, J=5.9 Hz, 2H), 3.51 (m, 2H), 3.46 (m, 2H), 3.28 (s, 3H), 2.80 (t, J=6.6 Hz, 2H), 2.54 (m, 4H), and 1.67 (m, 4H); MS (ESI) m/z 706.2 (M+H)⁺. HIRESIP

### Example 28

### 2,6-Difluoro-N-{3-[2-{[3-(4-morpholinyl)propyl]amino}-5-(2-{[3-O-pyrrolidinylmethyl)phenyl]amino}-4-pyrimidinyl)-1,3-thiazol-4-yl]phenyl}benzamide

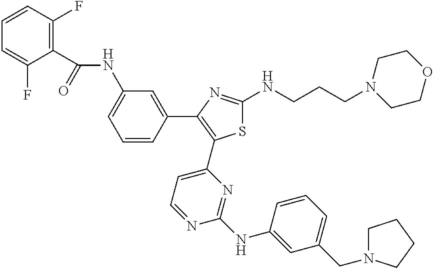

### Step A: N-[3-(5-(2-Chloro-4-pyrimidinyl)-2-{[3-(4-morpholinyl)propyl]amino}-1,3-thiazol-4-yl)phenyl]-2,6-difluorobenzamide

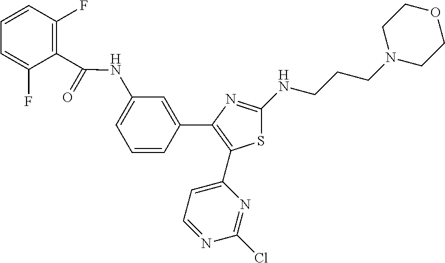

The title compound of Step A was prepared from N-{3-[(2-chloro-4-pyrimidinyl)acetyl]phenyl}-2,6-difluorobenzamide (0.4 g, 1.03 mmol), prepared by a procedure analogous to Example 1, Step C, NBS (0.183 g, 1.03 mmol) and N-[3-(4-morpholinyl)propyl]thiourea (0.231 g, 1.14 mmol), by a procedure analogous to Example 2, Step A. Yield 550 mg (93%). ¹H-NMR (300 MHz, DMSO-d₆) δ 10.99 (s, 1H), 10.82 (br, 1H), 8.75 (t, J=5.7 Hz, 1H), 8.31 (d, J=5.5 Hz, 1H), 7.94 (s, 1H), 7.76 (d, J=7.7 Hz, 1H), 7.59 (m, 1H), 7.47 (t, J=8.0 Hz, 1H), 7.32-7.21 (m, 3H), 6.89 (d, J=5.7 Hz, 1H), 3.92 (d, J=12.2 Hz, 1H), 3.75 (m, 2H), 3.46-3.36 (m, 4H), 3.16 (m, 2H), 3.04 (m, 2H), and 2.04 (m, 2H); MS (ESI) m/z 571 (M+H)⁺.

### Step B: 2,6-Difluoro-N-{3-[2-{[3-(4-morpholinyl)propyl]amino}-5-(2-{[3-(1-pyrrolidinylmethyl)phenyl]amino}-4-pyrimidinyl)-1,3-thiazol-4-yl]phenyl}benzamide

The title compound of Example 28 was prepared from N-[3-(5-(2-chloro-4-pyrimidinyl)-2-{[3-(4-morpholinyl)propyl]amino}-1,3-thiazol-4-yl)phenyl]-2,6-difluorobenzamide (0.12 g, 0.21 mmol) and 3-(1-pyrrolidinylmethyl)phenyl amine (0.038 g, 0.21 mmol) by a procedure analogous to Example 1, Step D. Yield 30 mg (25%): ¹H-NMR (400 MHz, DMSO-d₆) δ 10.90 (s, 1H), 9.44 (s, 1H), 8.29 (m, 1H), 8.09 (d, J=5.3 Hz, 1H), 7.86 (s, 1H), 7.78-7.71 (m, 2H), 7.63-7.53 (m, 2H), 7.44 (t, J=8.0 Hz, 1H), 7.29-7.21 (m, 3H), 7.18 (t, J=7.8 Hz, 1H), 6.88 (d, J=7.5 Hz, 1H), 6.31 (d, J=5.5 Hz, 1H), 3.54 (m, 6H), 3.30 (m, 2H), 2.42 (m, 4H), 2.33 (m, 6H), 1.75 (m, 2H), and 1.67 (m, 4H); HRMS C₃₈H₄₁N₆O₂F₂S (M+H)⁺ calcd 711.3041. found 711.3035.

### Example 29

### N-[3-(5-{2-[(3-{[2-(Dimethylamino)ethyl]oxy}phenyl)amino]-4-pyrimidinyl}-2-{[3-(4-morpholinyl)propyl]amino}-1,3-thiazol-4-yl)phenyl]-2,6-difluorobenzamide

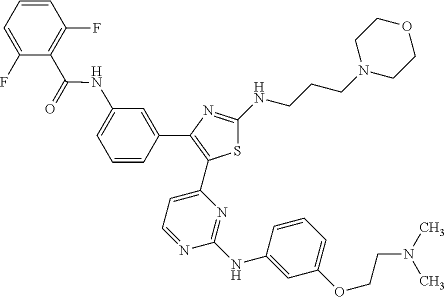

The title compound of Example 29 was prepared from N-[3-(5-(2-chloro-4-pyrimidinyl)-2-{[3-(4-morpholinyl)propyl]amino}-1,3-thiazol-4-yl)phenyl]-2,6-difluorobenzamide (0.12 g, 0.21 mmol), prepared by a procedure analogous to Example 28, Step A, and {2-[(3-aminophenyl)oxy]ethyl}dimethylamine hydrochloride (0.046 g, 0.21 mmol), by a procedure analogous to Example 1, Step D. Yield 77 mg (51%). ¹H-NMR (400 MHz, DMSO-d₆) δ 10.89 (s, 1H), 9.47 (s, 1H), 8.27 (t, J=5.5 Hz, 1H), 8.10 (d, J=5.3 Hz, 1H), 7.87 (s, 1H), 7.74 (d, J=7.8 Hz, 1H), 7.58 (m, 1 H), 7.49 (s, 1H), 7.44 (t, J=7.7 Hz, 1H), 7.32 (d, J=7.9 Hz, 1H), 7.28-7.20 (m, 3H), 7.15 (t, J=8.2 Hz, 1H), 6.53 (dd, J=7.9 and 2.3 Hz, 1H), 6.33 (d, J=5.4 Hz, 1H), 4.13 (t, J=5.2 Hz, 2H), 3.54 (m, 4H), 3.30 (m, 2H), 2.92 (br, 2H), 2.42 (s, 6H), 2.35 (m, 6H), and 1.74 (m, 2H); HRMS C₃₇H₄₁N₆O₃F₂S (M+H)⁺ calcd 715.2990. found 715.2997.

### Example 30

### 2,6-Difluoro-N-[3-(5-{2-[(3-fluorophenyl)amino]-4-pyrimidinyl}-2-{[3-(4-morpholinyl)propyl]amino}-1,3-thiazol-4-yl)phenyl]benzamide

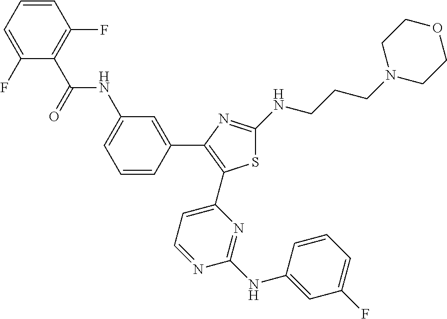

The title compound of Example 30 was prepared from N-[3-(5-(2-chloro-4-pyrimidinyl)-2-{[3-(4-morpholinyl)propyl]amino}-1,3-thiazol-4-yl)phenyl]-2,6-difluorobenzamide (0.11 g, 0.19 mmol), prepared by a procedure analogous to Example 28, Step A, and 3-fluorophenyl amine (0.021 g, 0.19 mmol), by a procedure analogous to Example 1, Step D. Yield 55 mg (52%). ¹H-NMR (400 MHz, DMSO-d₆) δ 10.89 (s, 1H), 9.74 (s, 1H), 8.34 (t, J=5.5 Hz, 1H), 8.14 (d, J=5.3 Hz, 1H), 7.88-7.80 (m, 2H), 7.77 (d, J=8.1 Hz, 1H), 7.59 (m, 1H), 7.48-7.41 (m, 2H), 7.31-7.21 (m, 4H), 6.73 (m, 1H), 6.38 (d, J=5.5 Hz, 1H), 3.54 (m, 4H), 3.32 (m, 2H), 2.34 (m, 6H). and 1.75 (m, 2H); HRMS C₃₃H₃₁F₃N₇O₂S (M+H)₊ calcd 646.2206. found 646.2208.

### Example 31

### 2,6-Difluoro-N-[3-(5-{2-[2-methyl-1,2,3,4-tetrahydro-7-isoquinolinyl)amino]-4-pyrimidinyl}-2-{[3-(4-morpholinyl)propyl]amino}-1,3-thiazol-4-yl)phenyl]benzamide

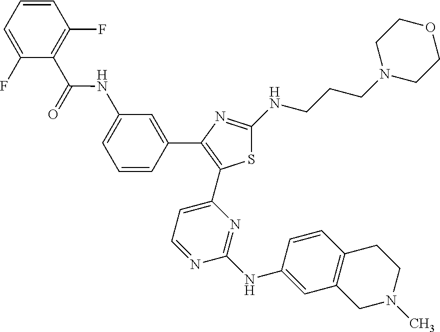

The title compound of Example 31 was prepared from N-[3-(5-(2-chloro-4-pyrimidinyl)-2-{[3-(4-morpholinyl)propyl]amino}-1,3-thiazol-4-yl)phenyl]-2,6-difluorobenzamide (0.11 g, 0.2 mmol), prepared by a procedure analogous to Example 28, Step A, and 2-methyl-1,2,3,4-tetrahydro-7-isoquinolinamine (0.032 g, 0.2 mmol), by a procedure analogous to Example 1, Step D. Yield 77 mg (55%). ¹H-NMR (400 MHz, DMSO-d₆) δ 10.89 (s, 1H), 9.35 (s, 1H), 8.28 (t, J=5.5 Hz, 1H), 8.07 (d, J=5.5 Hz, 1H), 7.85 (s, 1H), 7.75 (d, J=8.1 Hz, 1H), 7.62-7.54 (m, 2H), 7.43 (t, J=8.0 Hz, 1H), 7.37 (d, J=8.0 Hz, 1H), 7.28-7.20 (m, 3H), 6.97 (d, J=8.1 Hz, 1H), 6.29 (d, J=5.4 Hz, 1H), 3.54 (m, 4H), 3.47 (s, 2H), 3.30 (m, 2H), 2.73 (m, 2H), 2.57 (m, 2H), 2.33 (m, 9H), and 1.75 (m, 2H); HRMS C₃H₃₉N₈O₂F₂S (M+H)⁺ calcd 697.2885. found 697.2872.

### Example 32

### N-{3-[2-(cyclopropylamino)-5-(2-{[3-{[2-(dimethylamino)ethyl]oxy}-4-(methyloxy)phenyl]amino}-4-pyrimidinyl)-1,3-thiazol-4-yl]phenyl}-2,6-difluorobenzamide

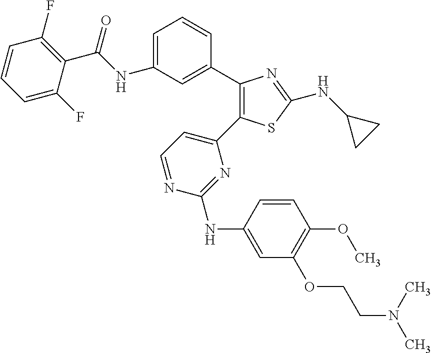

### Step A: N-Cyclopropylthiourea

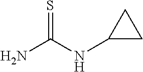

To obtain the title compound of Step A, cyclopropylamine (5.0 g, 87 mmol) in THF (5 mL) was placed in a round bottom flask at 0° C. and 4 N HCl in dioxane (22 mL, 87 mmol) was added drop-wise over 30 min. This mixture was then allowed to stir for 30 min at rt. Potassium thiocyanate (8.5 g, 87 mmol) dissolved in 5 mL H₂O was then added in one portion to the stirring solution of cyclopropylamine hydrochloride. This mixture was stirred 1 h at rt and then 2.5 h at 85° C., followed by stirring at rt overnight. The reaction was then concentrated to dryness. MeOH (50 mL) was added to the concentrated reaction and solids that persisted were filtered away. Subsequent concentration of the MeOH solution yielded 10.1 g of the crude cyclopropyl thiourea. ¹H-NMR (400 MHz, DMSO-d₆) δ ppm 8.03 (bs, 3H), 2.55 (s, 1H), and 0.67 (m, 4H).

### Step B: N-{3-[5-(2-Chloro-4-pyrimidinyl)-2-(cyclopropylamino)-1,3-thiazol-4-yl]phenyl}-2,6-difluorobenzamide

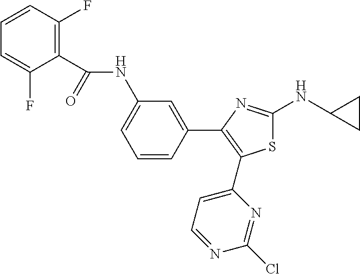

To obtain the desired compound, N-{3-[(E)-2-(2-chloro-4-pyrimidinyl)-1-hydroxyethenyl]phenyl}-2,6-difluorobenzamide (2 g, 5.18 mmol), prepared by a procedure analogous to Example 1, Step C, was placed in a round bottom flask with DMF (20 mL). NBS (0.92 g, 5.18 mmol) was added and the resulting mixture was stirred at rt for 10 min. N-cyclopropylthiourea (1.2 g, 10.3 mmol) was then added and stirring at rt was continued for 1 h. EtOAc and H₂O were added to the reaction mixture and the desired product was extracted into the organic phase which was then concentrated onto silica gel and purified via column chromatography to yield fractions which were concentrated to dryness. This material was then sonicated in ether and the solid that persisted was filtered off to yield 500 mg of the title compound of Step B. (20%). ¹H-NMR (400 MHz, DMSO-d₆) δ ppm 10.90 (s, 1H), 8.92 (s, 1H), 8.28 (d, J=6.0 Hz, 1H), 7.87 (s, 1H), 7.73 (d, J=9.2 Hz, 1H), 7.51-7.02 (m, 1H), 7.44 (t, J=7.9 Hz, 1H), 7.20-7.28 (m, 3H), 6.86 (d, J=5.7 Hz, 1H), 2.61 (s, 1H), 0.75-0.82 (m, 2H), and 0.54-0.62 (m, 2H).

### Step C: N-{3-[2-(cyclopropylamino)-5-(2-{[3-{[2-(dimethylamino)ethyl]oxy}-4-(methyloxy)phenyl]amino}-4-pyrimidinyl)-1,3-thiazol-4-yl]phenyl}-2,6-difluorobenzamide

To obtain the title compound of Example 32, N-{3-[5-(2-chloro-4-pyrimidinyl)-2-(cyclopropylamino)-1,3-thiazol-4-yl]phenyl}-2,6-difluorobenzamide (0.1 g, 0.207 mmol) and 3-{[2-(dimethylamino)ethyl]oxy}-4-(methyloxy)phenyl amine (0.044 g, 0.207 mmol) were combined with i-PrOH (2 mL) and concentrated HCl (2 drops) in a microwave vial. The reaction was heated to 180° C. for 20 min in the microwave then cooled to rt. TEA (approx. 0.1 mL) and silica gel were combined with the reaction and the resulting mixture was concentrated to dryness. Column chromatography using EtOAc. MeOH, and NH₄OH yielded fractions which were concentrated to dryness. This material was then further purified on a RP acidic HPLC. The resulting fractions were free-based via extraction and concentrated to dryness to yield 56 mg of the title compound of Example 32 (41% Y). ¹H-NMR (400 MHz, DMSO-d₆) δ 10.80 (s, 1), 9.28 (s, 1H), 8.59 (s, 1H), 8.06 (d, J=5.6 Hz, 1H), 7.86 (s, 1H), 7.72 (d, J=8.1 Hz, 1H), 7.57 (m, 1H), 7.53 (d, J=2.7 Hz, 1H), 7.42 (t, J=7.9 Hz, 1H), 7.26-7.19 (m, 3H), 7.17 (dd, J=8.8 and 2.1 Hz, 1H), 6.84 (d, J=8.9 Hz, 1H), 6.27 (d, J=5.4 Hz, 1H), 4.06 (t, J=5.9 Hz, 2H), 3.69 (s, 3H), 2.66 (m, 2H), 2.60 (m, 1H), 2.22 (s, 6H), 0.76 (m, 2H), and 0.58 (m, 2H); HRMS C₃₄H₃₄N₇O₃F₂S (M+H)⁺ calcd 658.2412. found 658.2424.

### Example 33

### N-[3-(2-(Cyclopropylamino)-5-{2-[(3-fluoro-4-{[2-(1-pyrrolidinyl)ethyl]oxy}phenyl)amino]-4-pyrimidinyl}-1,3-thiazol-4-yl)phenyl]-2,6-difluorobenzamide

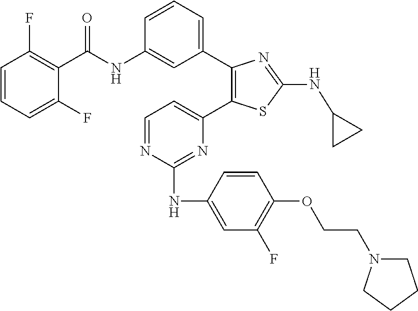

### Step A: 1-{2-[(2-Fluoro-4-nitrophenyl)oxy]ethyl}pyrrolidine

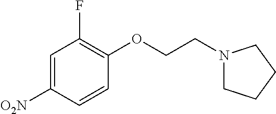

To obtain the target compound, NaH (2.5 g, 63 mmol, 60% dispersion) was placed in a round bottom flask with THF (25 mL) at 0° C. 2-(1-Pyrrolidinyl)ethanol (7.5 mL, 68 mmol) was added portion-wise to the stirring NaH solution over 15 min. This reaction mixture was stirred at 0° C. for 45 min. 3,4-Difluoronitrobenzene (10 g, 63 mmol) was then added portion-wise to the reaction at 0° C. The reaction was allowed to stir and warm to it over several hours. A few drops of MeOH were added to the reaction. EtOAc and H₂O were then added to the reaction mixture and the desired product was extracted into the organic phase which was then concentrated onto silica gel and purified via column chromatography to yield 8.5 g of the target compound of Step A. (53%) ¹H-NMR (400 MHz, DMSO-d₆) δ ppm 8.06-8.15 (m, 2H), 7.40 (t, J=8.8 Hz, 1H), 4.28 (t, J=5.7 Hz, 2H), 2.82 (t, J=5.7 Hz, 2H), 2.5 (m, 4H), and 1.65 (m, 4H).

### Step B: 3-Fluoro-4-{[2-(1-pyrrolidinyl)ethyl]oxy}phenyl amine hydrochloride

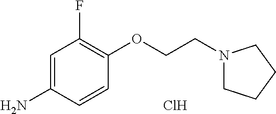

To obtain the desired target 1-{2-[(2-fluoro-4-nitrophenyl)oxy]ethyl}pyrrolidine (8.5 g, 33 mmol) and EtOH (50 mL) were placed in a pressure reaction vessel under N₂. Platinum on carbon (500 mg, 5% by wt) was added followed by 40 psi of H₂. The reaction was stirred overnight at rt. The reaction was then filtered through celite and the subsequent filtrate was concentrated to dryness. A small amount of EtOAc was added followed by 4 N HCl in dioxane (8.25 mL, 33 mmol). This was then concentrated to dryness to yield a sticky solid which was then sonicated in ether and filtered off as a beige powder to yield 8.0 g of the desired phenyl amine as a hydrochloride salt. ¹H-NMR (400 MHz, DMSO-d₆) δ ppm 6.92 (m, 1H), 6.41 (m, 1H), 6.31 (m, 1H), 5.28 (s, 2H), 4.20 (m, 2H), 3.54 (s, 2H), 3.47 (m, 2H), 3.06 (s, 2 H), and 1.97 (s, 2H), 1.86 (s, 2H).

### Step C: N-[3-(2-(Cyclopropylamino)-5-{2-[(3-fluoro-4-{[2-(1-pyrrolidinyl)ethyl]oxy}phenyl)amino]-4-pyrimidinyl}-1,3-thiazol-4-yl)phenyl]-2,6-difluorobenzamide

To obtain the title compound of Example 33, 4-[N-{3-[5-(2-chloro-4-pyrimidinyl)-2-(cyclopropylamino)-1,3-thiazol-4-yl]phenyl}-2,6-difluorobenzamide (0.1 g, 0.207 mmol), prepared by a procedure analogous to Example 32, Step B, and 3-fluoro-4-{[2/1-pyrrolidinyl)ethyl]oxy}phenyl amine (0.054 g, 0.207 mmol) were combined with i-PrOH (2 mL) and concentrated HCl (2 drops) in a microwave vial. The reaction was heated to 180° C. for 15 min in the microwave then cooled to rt. TEA (approx. 0.1 mL) and silica gel were combined with the reaction and the resulting mixture was concentrated to dryness. Column chromatography using EtOAc. MeOH and NH₄OH yielded fractions which were concentrated to dryness. This material was then further purified on a RP acidic HPLC. The resulting fractions were free-based via extraction and concentrated to dryness. This material was then sonicated in ether and the solid that persisted was filtered off to yield 40 mg of the title compound of Example 33 (29% Y). ¹H-NMR (400 MHz, DMSO-d₆) δ 10.89 (s, 1H), 9.52 (s, 1H), 8.63 (s, 1H), 8.09 (d, J=5.3 Hz, 1H), 7.86 (s, 1H), 7.84 (d, J=12.6 Hz, 1H), 7.72 (d, J=7.8 Hz, 1H), 7.56 (m, 1H), 7.42 (t, J=7.8 Hz, 1H), 7.32 (d, J=8.5 Hz, 1H), 7.26-7.19 (m, 3 H), 7.05 (t, J=9.4 Hz, 1H), 6.32 (d, J=5.4 Hz, 1H), 4.06 (t, J=5.8 Hz, 2H), 2.78 (brs, 2H), 2.60 (brs, 1H), 2.52 (brs, 4H), 1.66 (brs, 4H), 0.76 (m, 2H), and 0.60 (m, 2H); HRMS C₃₆H₃₃F₃N₇O₂S (M+H)⁺ calcd 672.2363. found 672.2365.

### Example 34

### N-{3-[2-(Acetylamino)-5-(2-{[3-(1-pyrrolidinylmethyl)phenyl]amino}-4-pyrimidinyl)-1,3-thiazol-4-yl]phenyl}-2,6-difluorobenzamide

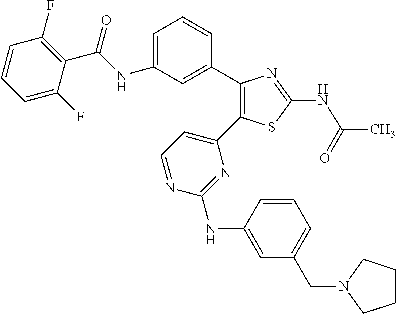

Step A: N-{3-[2-(Acetylamino)-5-(2-chloro-4-pyrimidinyl)-1,3-thiazol-4-yl]phenyl}-2,6-difluorobenzamide

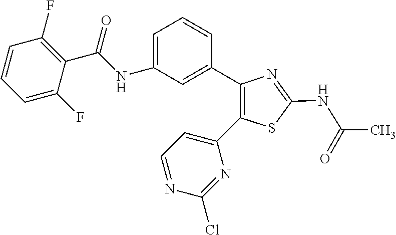

The title compound of Step A was prepared from N-{3-[(E)-2-(2-chloro-4-pyrimidinyl)-1-hydroxyethenyl]phenyl}-2,6-difluorobenzamide (0.4 g, 1.03 mmol), prepared by a procedure analogous to Example 1, Step C. NBS (0.18 g, 1.03 mmol) and N-(aminocarbonothioyl)acetamide (0.121 mg, 1.03 mmol), by a procedure analogous to Example 2, Step A. Yield 0.15 g (30%). ¹H-NMR (400 MHz, DMSO-d₆) δ 12.62 (s, 1 H), 10.92 (s, 1H), 8.49 (d, J=5.5 Hz, 1H), 7.98 (s, 1H), 7.73 (d, J=8.1 Hz, 1H), 7.58 (m, 1H), 7.46 (t, J=8.0 Hz, 1H), 7.33 (d, J=7.9 Hz, 1H), 7.28-7.21 (m, 2H), 7.15 (d, J=5.4 Hz, 1H), and 2.20 (s, 3H); MS (ESI) m/z 486.04 (M+H)⁺.

### Step B: N-{3-[2-(Acetylamino)-5-(2-{[3-(1-pyrrolidinylmethyl)phenyl]amino}-4-pyrimidinyl)-1,3-thiazol-4-yl]phenyl}-2,6-difluorobenzamide

The title compound of Example 34 was prepared from N-{3-[2-(acetylamino)-5-(2-chloro-4-pyrimidinyl)-1,3-thiazol-4-yl]phenyl}-2,6-difluorobenzamide (0.075 g, 0.12 mmol) and 3-(1-pyrrolidinylmethyl)phenyl amine (0.027 g, 0.12 mmol) by a procedure analogous to Example 1, Step D. Yield 0.04 g (41%). ¹H-NMR (400 MHz, DMSO-d₆) δ 12.43 (s, 1H), 10.90 (s, 1H), 9.62 (s, 1H), 8.24 (d, J=5.3 Hz, 1H), 7.99 (s, 1H), 7.78 (brs, 1H), 7.72 (d, J=8.4 Hz, 1H), 7.63-7.53 (m, 2H), 7.45 (t, J=7.9 Hz, 1H), 7.31 (d, J=7.5 Hz, 1H), 7.27-7.20 (m, 2H), 7.18 (t, J=7.9 Hz, 1H), 6.89 (d, J=7.4 Hz, 1H), 6.52 (d, J=5.3 Hz, 1H), 3.57 (brs, 2H), 2.43 (brs, 4H), 2.18 (s, 3H), and 1.66 (brs, 4H); MS (ESI) m/z 626.35 (M+H)⁺. HRMS C₃₃H₃₀F₂N₇O₂S (M+H)⁺ calcd 626.215. found 626.215.

### Example 35

### N-[3-(5-{2-[(4-{[2-(Dimethylamino)ethyl]oxy}phenyl)amino]-4-pyrimidinyl}-2-{[(2,2,2-trifluoroethyl)sulfonyl]amino}-1,3-thiazol-4-yl)phenyl]-2,6-difluorobenzamide

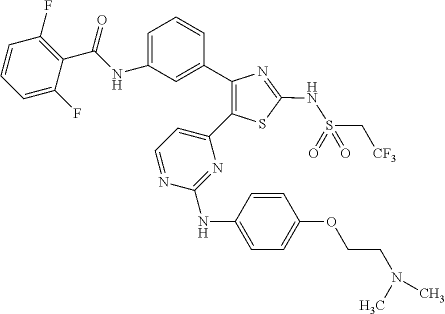

### Step A: N-[3-(5-(2-Chloro-4-pyrimidinyl)-2-{[(2,2,2-trifluoroethyl)sulfonyl]amino}-1,3-thiazol-4-yl)phenyl]-2,6-difluorobenzamide

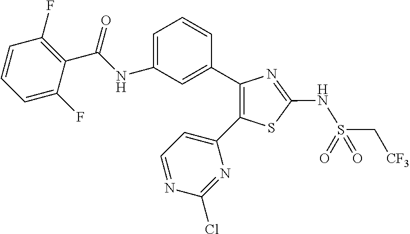

To obtain the desired compound N-{3-[2-amino-5-(2-chloro-4-pyrimidinyl)-1,3-thiazol-4-yl]phenyl}-2,6-difluorobenzamide (0.5 g, 1.12 mmol), prepared by a procedure analogous to Example 2, Step A, was stirred in DCM (8 mL) followed by addition of 2,2,2-trifluoroethanesulfonyl chloride (0.31 g, 1.7 mmol) in one portion. 2,6 Lutidine (0.14 mL, 1.24 mmol) was then added drop-wise and the mixture was allowed to stir at rt for 1 h. THF (2 mL) was added to assist with solubility followed by additional 2,6 lutidine (0.14 mL, 1.24 mmol). The subsequent mixture was heated at 40° C. for several hours until by LC/MS the reaction was complete. EtOAc and H₂O were added to the reaction mixture and the desired product was extracted into the organic phase which was then concentrated onto silica gel. Column chromatography using EtOAc and hexane yielded pure fractions which were combined and concentrated to give 0.45 g of the target compound (68% Yield). ¹H-NMR (400 MHz, DMSO-d₆) δ 11.06 (s, 1H), 8.50 (d, J=5.5 Hz, 1H), 7.94 (s, 1H), 7.85 (d, J=8.3 Hz, 1H), 7.64-7.54 (m, 2H), 7.37 (d, J=7.8 Hz, 1H), 7.29-7.22 (m, 2H), 6.90 (d, J=5.8 Hz, 1H), and 4.47 (q, J=10.2 Hz, 2H); MS (ESI) m/z 590.01 (M+H)⁺.

### Step B: N-[3-(5-{2-[(4-{[2-(Dimethylamino)ethyl]oxy}phenyl)amino]-4-pyrimidinyl}-2-{[(2,2,2-trifluoroethyl)sulfonyl]amino}-1,3-thiazol-4-yl)phenyl]-2,6-difluorobenzamide

The title compound of Example 35 was prepared from N-[3-(5-(2-chloro-4-pyrimidinyl)-2-{[(2,2,2-trifluoroethyl)sulfonyl]amino}-1,3-thiazol-4-yl)phenyl]-2,6-difluorobenzamide (0.107 g, 0.18 mmol) and 4-{[2-(dimethylamino)ethyl]oxy}-N-methylphenyl amine (0.039 g, 0.18 mmol) by a procedure analogous to Example 1, Step D. Yield 0.035 g (26%). ¹H-NMR (400 MHz, DMSO-d₆) δ 10.87 (s, 1H), 9.29 (s, 1H), 8.03 (d, J=5.7 Hz, 1H), 7.79 (d, J=8.0 Hz, 1H), 7.76 (s, 1H), 7.65 (d, J=9.1 Hz, 2H), 7.57 (m, 1H), 7.40 (t, J=7.9 Hz, 1H), 7.27-7.20 (m, 3H), 6.87 (d, J=9.2 Hz, 2H), 6.28 (d, J=5.3 Hz, 1H), 4.13 (br, 2H), 4.04 (q, J=10.5 Hz, 2H), 3.08 (brs, 2H), and 2.56 (brs, 6H); HRMS Calcd for C₃₂H₂₉N₇O₄F₅S₂ (M+H)⁺: 734.1643. Found: 734.1620.

### Example 36

### N-[3-(2-(Dimethylamino)-5-{2-[(4-{[2-(dimethylamino)ethyl]oxy}phenyl)amino]-4-pyrimidinyl}-1,3-thiazol-4-yl)phenyl]-2,6-difluorobenzamide

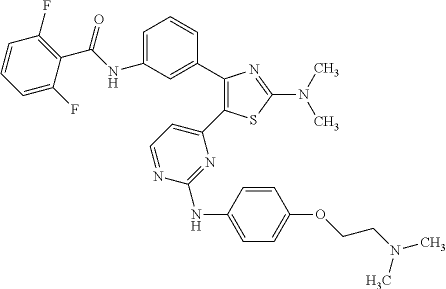

### Step A: N,N-Dimethylthiourea

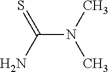

To obtain the title compound of Step A, 2 M dimethyl amine in THF (5 mL) was placed in a round bottom flask and 4 N HCl in dioxane (2.5 mL, 10 mmol) was added drop-wise over 15 min. Potassium thiocyanate (0.97 g, 10 mmol) dissolved in 1 mL H₂O was then added in one portion to the stirring solution of dimethyl amine hydrochloride. This mixture was then allowed to stir 16 h at rt and concentrated to dryness. MeOH (50 mL) was added to the concentrated reaction and solids that persisted were filtered away and subsequent concentration of the MeOH solution yielded 1.1 g of the crude thiourea. ¹H-NMR (400 MHz, DMSO-d₆) δ 8.18 (brs, 2H), and 2.51 (t, J=5.9 Hz, 6H).

### Step B: N-{3-[5-(2-Chloro-4-pyrimidinyl)-2-(dimethylamino)-1,3-thiazol-4-yl]phenyl}-2,6-difluorobenzamide

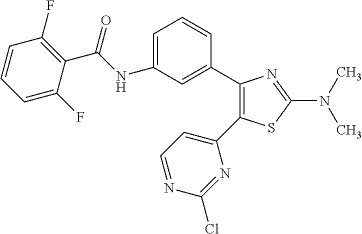

The title compound of Step B was prepared from N-{3-[(E)-2-(2-chloro-4-pyrimidinyl)-1-hydroxyethenyl]phenyl}-2,6-difluorobenzamide (1.85 g, 4.8 mmol), prepared by a procedure analogous to Example 1, Step C, NBS (0.853 g, 4.8 mmol) and N,N-dimethylthiourea (1.0 g, 9.6 mmol), by a procedure analogous to Example 2, Step A. Yield 0.85 g (38%). ¹H-NMR (400 MHz, DMSO-d₆) δ 10.94 (s, 1H), 8.29 (d, J=6.0

Hz, 1H), 7.85 (s, 1H), 7.81 (d, J=9.1 Hz, 1H), 7.59 (m, 1H), 7.48 (t, J=8.0 Hz, 1H), 7.29-7.21 (m, 3H), 6.83 (d, J=6.0 Hz, 1H), and 3.15 (s, 6H); MS (ESI) m/z 472.10 (M+H)⁺.

### Step C: N-[3-(2-(Dimethylamino)-5-{2-[(4-{[2-(dimethylamino)ethyl]oxy}phenyl)amino]-4-pyrimidinyl}-1,3-thiazol-4-yl)phenyl]-2,6-difluorobenzamide

The title compound of Example 36 was prepared from N-{3-[5-(2-chloro-4-pyrimidinyl)-2-(dimethylamino)-1,3-thiazol-4-yl]phenyl}-2,6-difluorobenzamide (0.1 g, 0.21 mmol) and 4-{[2-(dimethylamino)ethyl]oxy}phenyl amine (0.046 g, 0.21 mmol) by a procedure analogous to Example 1, Step D. Yield 0.070 g (54%). ¹H-NMR (400 MHz, DMSO-d₆) δ 10.91 (s, 1H), 9.29 (s, 1H), 8.05 (d, J=5.5 Hz, 1H), 7.83 (s, 1H), 7.79 (d, J=8.5 Hz, 1H), 7.63-7.53 (m, 3H), 7.45 (t, J=7.9 Hz, 1H), 7.28-7.20 (m, 3H), 6.85 (d, J=8.9 Hz, 2H), 6.23 (d, J=5.4 Hz, 1H), 3.98 (t, J=6.5 Hz, 2H), 3.12 (s, 6H), 2.59 (t, J=6.2 Hz, 2H), and 2.20 (s, 6H); MS (ESI) m/z 616.23 (M+H)⁺.

### Example 37

### N-{3-[5-{2-[(3-Chloro-4-{[2-(dimethylamino)ethyl]oxy}phenyl)amino]-4-pyrimidinyl}-2-(dimethylamino)-1,3-thiazol-4-yl]phenyl}-2,6-difluorobenzamide

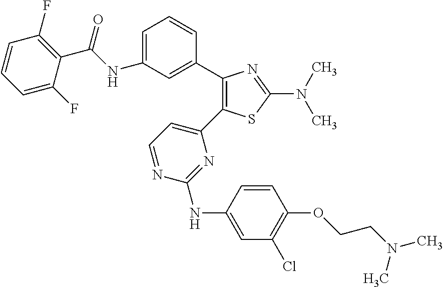

The title compound of Example 37 was prepared from N-{3-[5-(2-chloro-4-pyrimidinyl)-2-(dimethylamino)-1,3-thiazol-4-yl]phenyl}-2,6-difluorobenzamide (0.1 g, 0.21 mmol), prepared by a procedure analogous to Example 36, Step C, and 3-chloro-4-{[2-(dimethylamino)ethyl]oxy}phenyl amine (0.53 g, 0.21 mmol), by a procedure analogous to Example 1, Step D. Yield 0.097 g (70%). ¹H-NMR (400 MHz, DMSO-d₆) δ 10.90 (s, 1H), 9.50 (s, 1H), 8.08 (d, J=5.3 Hz, 2H), 7.82 (s, 1H), 7.78 (d, J=8.3 Hz, 1H), 7.56 (m, 1H), 7.48-7.41 (m, 2H), 7.27-7.19 (m, 3H), 7.07 (d, J=9.2 Hz, 1H), 6.27 (d, J=5.7 Hz, 1H), 4.05 (t, J=6.0 Hz, 2H), 3.11 (s, 6H), 2.61 (t, J=6.2 Hz, 2H), and 2.20 (s, 6H); MS (ESI) m/z 650.18 (M+H)⁺.

### Example 38

### N-[3-(2-(Dimethylamino)-5-{2-[2-methyl-1,2,3,4-tetrahydro-7-isoquinolinyl)amino]-4-pyrimidinyl}-1,3-thiazol-4-yl)phenyl]-2,6-difluorobenzamide

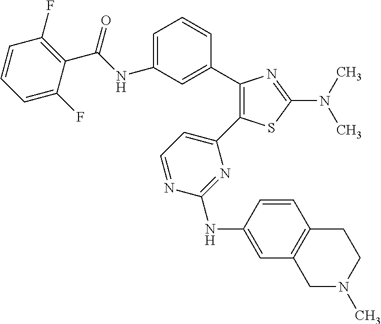

The title compound of Example 38 was prepared from N-{3-[5-(2-chloro-4-pyrimidinyl)-2-(dimethylamino)-1,3-thiazol-4-yl]phenyl}-2,6-difluorobenzamide (0.1 g, 0.21 mmol), prepared by a procedure analogous to Example 36, Step C, and 2-methyl-1,2,3,4-tetrahydro-7-isoquinolinamine (0.034 g, 0.21 mmol), by a procedure analogous to Example 1, Step D. Yield 0.061 g (48%). ¹H-NMR (400 MHz, DMSO-d₆) δ 10.92 (s, 1H), 9.39 (s, 1H), 8.08 (d, J=5.3 Hz, 1H), 7.84 (s, 1H), 7.80 (d, J=7.9 Hz, 1H), 7.67 (s, 1H), 7.58 (m, 1H), 7.46 (t, J=7.9 Hz, 1H), 7.34 (d, J=8.7 Hz, 1H), 7.29-7.21 (m, 3H), 6.98 (d, J=8.5 Hz, 1H), 6.27 (d, J=5.6 Hz, 1H), 3.47 (s, 2H), 3.14 (s, 6H), 2.74 (m, 2H), 2.56 (m, 2H), and 2.31 (s, 3H); MS (ESI) m/z 598.2 (M+H)⁺. HRMS C₃₂H₃₀F₂N₇OS (M+H)⁺ calcd 598.2195. found 598.2194.

### Example 39

### N-{3-[2-(Dimethylamino)-5-(2-{[3-(1-pyrrolidinylmethyl)phenyl]amino}-4-pyrimidinyl)-1,3-thiazol-4-yl]phenyl}-2,6-difluorobenzamide

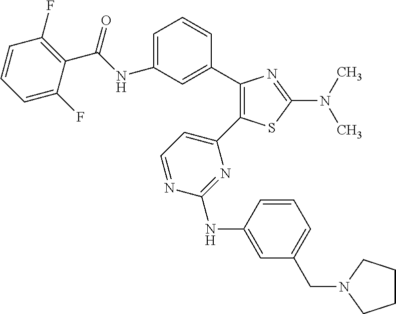

The title compound of Example 39 was prepared from N-{3-[5-(2-chloro-4-pyrimidinyl)-2-(dimethylamino)-1,3-thiazol-4-yl]phenyl}-2,6-difluorobenzamide (0.13 g, 0.27 mmol), prepared by a procedure analogous to Example 36, Step C, and 3-(1-pyrrolidinylmethyl)phenyl amine (0.048 g, 0.27 mmol), by a procedure analogous to Example 1, Step D. Yield 0.07 g (42%). ¹H-NMR (400 MHz, DMSO-d₆) δ 10.93 (s, 1H), 9.48 (s, 1H), 8.10 (d, J=5.4 Hz, 1H), 7.88 (s, 1H), 7.84 (s, 1H), 7.80 (d, J=8.7 Hz, 1H), 7.58 (m, 1H), 7.52-7.44 (m, 2H), 7.29-7.17 (m, 4H), 6.89 (d, J=7.2 Hz, 1H), 6.28 (d, J=5.3 Hz, 1H), 3.56 (s, 2H), 3.14 (s, 6H), 2.43 (m, 4H), and 1.67 (m, 4H); MS (ESI) m/z 612.2 (M+H)⁺.

### Example 40

### N-{3-[5-{2-[(3-Chloro-4-{[2-(1-pyrrolidinyl)ethyl]oxy}phenyl)amino]-4-pyrimidinyl}-2-(dimethylamino)-1,3-thiazol-4-yl]phenyl}-2,6-difluorobenzamide

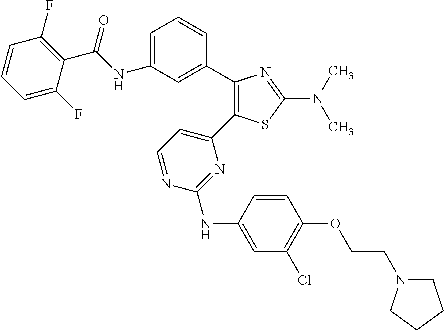

The title compound of Example 40 was prepared from N-{3-[5-(2-chloro-4-pyrimidinyl)-2-(dimethylamino)-1,3-thiazol-4-yl]phenyl}-2,6-difluorobenzamide (0.12 g, 0.25 mmol), prepared by a procedure analogous to Example 36, Step A, and 3-chloro-4-{[2-(1-pyrrolidinyl)ethyl]oxy}phenyl amine (0.068 g, 0.25 mmol), by a procedure analogous to Example 1, Step D. Yield 0.086 g (51%). ¹H-NMR (400 MHz, DMSO-d₆) δ 10.93 (s, 1H), 9.52 (s, 1H), 8.10 (d, J=5.5 Hz, 2H), 7.84 (s, 1 H), 7.79 (d, J=7.8 Hz, 1H), 7.58 (m, 1H), 7.50-7.43 (m, 2H), 7.28-7.21 (m, 3H), 7.08 (d, J=9.3 Hz, 1H), 6.28 (d, J=5.6 Hz, 1H), 4.08 (t, J=6.1 Hz, 2H), 3.13 (s, 6H), 2.79 (t, J=6.5 Hz, 2H), 2.54 (m, 4H), and 1.66 (m, 4H); MS (ESI) m/z 676.26 (M+H)⁺. HRMS C₃₄H₃₃ClF₂N₇O₂S (M+H)⁺ calcd 676.2068. found 676.2064.

### Example 41

### N-{3-[5-(2-{[3-Chloro-4-(4-morpholinyl)phenyl]amino}-4-pyrimidinyl)-2-(dimethylamino)-1,3-thiazol-4-yl]phenyl}-2,6-difluorobenzamide trifluoroacetate

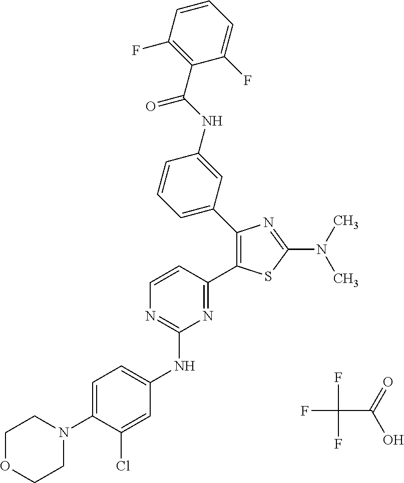

The title compound of Example 41 was synthesized using standard microwave chloride displacement conditions as in Example 1, Step D in trifluoroethanol using N-{3-[5-(2-chloro-4-pyrimidinyl)-2-(dimethylamino)-1,3-thiazol-4-yl]phenyl}-2,6-difluorobenzamide (0.100 g, 0.21 mmol), prepared in a procedure analogous to Example 36, Step C, and 3-chloro-4-(4-morpholinyl)phenyl amine (0.054 g, 0.25 mmol). The solvent was removed and the residue was taken up in DMSO/MeOH (2:1) and purified via HPLC. The desired fractions were combined and solvent removed to give 0.063 g, 46% yield, of the title compound of Example 41 as a pale orange solid. ¹H-NMR (400 MHz, DMSO-d₆) δ 10.93 (s, 1H), 9.61 (s, 1H), 8.07-8.17 (m, 2H), 7.84 (d, J=1.8 Hz, 1H), 7.78 (s, 1H), 7.53-7.63 (m, 1H), 7.41-7.52 (m, 2H), 7.21-7.28 (m, 3H), 7.10 (d, J=8.8 Hz, 1H), 6.30 (d, J=5.5 Hz, 1H), 3.68-3.76 (m, 4H), 3.14 (s, 6H), and 2.86-2.93 (m, 4H); ES-LCMS m/z 648 (M+H).

### Example 42

### N-{3-[2-(Dimethylamino)-5-(2-{[3-methyl-4-(4-piperidinylmethyl)phenyl]amino}-4-pyrimidinyl)-1,3-thiazol-4-yl]phenyl}-2,6-difluorobenzamide

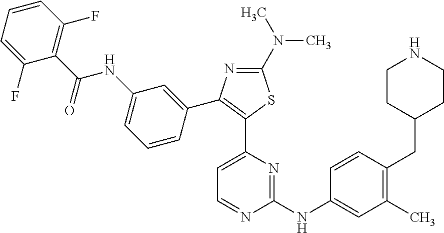

### Step A: 1,1-Dimethylethyl 4-(iodomethyl)-1-piperidinecarboxylate

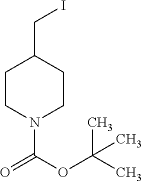

To a solution of 1,1-dimethylethyl 4-(hydroxymethyl)-1-piperidinecarboxylate (5.0 g, 23.22 mmol), triphenylphosphine (7.3 g, 27.87 mmol) and imidazole (1.4 g, 27.87 mmol) in THF (20 mL) at 0° C. was added slowly via an addition funnel, iodine (7.2 g, 27.87 mmol) in THF (15 mL). The reaction was allowed to warm to rt and stirred for 3 h. The reaction was diluted with 10% EtOAc in hexane and filtered through a pad of silica with copious 10% EtOAc in hexane washings. Silica was added and the volatiles were evaporated under reduced pressure and the residue was purified by flash column chromatography (0 to 25% EtOAc:hexane) to afford 5.5 g of the title compound of Step A. ¹H-NMR (400 MHz, CHLOROFORM-d) δ 3.96-4.19 (m, 2H), 3.05 (d, J=6.4 Hz, 2H), 2.56-2.71 (m, 2H), 1.78 (d, J=13.4 Hz, 2H), 1.50-1.64 (m, 1H), 1.40 (s, 9H), and 1.01-1.16 (m, 2H).

### Step B: [(1-{[(1,1-Dimethylethyl)oxy]carbonyl}-4-piperidinyl)methyl](iodo)zinc

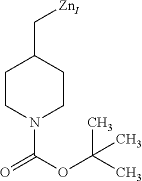

To a slurry of zinc dust (1.38 g, 21.1 mmol) and celpure P65 celite (0.27 g) in DMF (3.4 mL) was added a 7:5 v/v mixture of chlorotrimethylsilane:1,2-dibromoethane (0.45 mL) over a ten min period at rt. The internal temperature was maintained below 65° C. during the addition. The slurry was stirred for 15 min and a solution of 1,1-dimethylethyl-4-(iodomethyl)-1-piperidinecarboxylate (5.50 g, 17.0 mmol) in DMF (8.5 mL) was added slowly at a rate to maintain temperature below 65° C. The reaction mixture was heated at 65° C. for 5 min and allowed to cool to rt with stirring for 30 min. The mixture was filtered to afford a solution of the desired product in DMF.

### Step C: [3-Methyl-4-(4-piperidinylmethyl)phenyl]amine 3-methyl-4-(4-piperidinylmethyl)phenyl amine

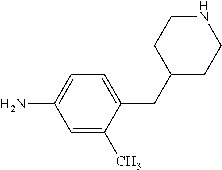

To 1-bromo-2-methyl-4-nitrobenzene (1 g, 4.63 mmol) was added [1,1-bis(diphenylphosphino)-ferrocene]dichloropalladium(II)-DCM complex (0.38 g, 0.46 mmol), cuprous iodide (0.18 g, 0.92 mmol), DMA (6 mL), and (1-{[(1,1-dimethylethyl)oxy]carbonyl}-4-piperidinyl)(iodo)zinc solution from Step B (9 mL). The resulting mixture was purged with N₂ and heated to 80° C. for 2 h. EtOAc was added and the mixture was filtered through a pad of celite and washed with EtOAc. The organic layer was washed with water and dried over Na₂SO₄ and the volatiles were evaporated under reduced pressure and HOAc and iron powder were added to the residue. The reaction mixture was heated to 60° C. for 1 h. MeOH was added and the mixture was filtered through a pad of celite and washed with MeOH. Silica was added and the volatiles were evaporated under reduced pressure. The residue was purified by flash chromatography with (84% DCM, 15% MeOH, and 1% NH₄OH): DCM 0% to 100% to afford 0.54 g of the title compound of Step C. MS (ESI): 205 [M+H]⁺.

### Step D: N-{3-[2-(Dimethylamino)-5-(2-{[3-methyl-4-(4-piperidinylmethyl)phenyl]amino}-4-pyrimidinyl)-1,3-thiazol-4-yl]phenyl}-2,6-difluorobenzamide

The title compound of Example 42 was made following the general procedure from Example 1, Step D with N-{3-[5-(2-chloro-4-pyrimidinyl)-2-(dimethylamino)-1,3-thiazol-4-yl]phenyl}-2,6-difluorobenzamide (0.20 g, 0.42 mmol), prepared by a procedure analogous to Example 36, Step C, and [3-methyl-4-(4-piperidinylmethyl)phenyl]amine 3-methyl-4-(4-piperidinylmethyl)aniline (0.095 g, 0.47 mmol). ¹H-NMR (400 MHz, DMSO-d₆) δ 10.93 (s, 1H), 9.34 (s, 1H), 8.09 (d, J=5.5 Hz, 1H), 7.86 (s, 1H), 7.81 (d, J=7.5 Hz, 1H), 7.72 (s, 1H), 7.54-7.65 (m, 1H), 7.47 (t, J=7.9 Hz, 1H), 7.36 (d, J=9.7 Hz, 1H), 7.20-7.31 (m, 3H), 6.96 (d, J=8.2 Hz, 1H), 6.28 (d, J=5.5 Hz, 1H), 3.32 (s, 2H), 3.15 (s, 6H), 2.89 (d, J=11.7 Hz, 2H), 2.32-2.45 (m, 4H), 2.27 (s, 3H), 1.51 (d, J=10.3 Hz, 2H), and 0.98-1.19 (m, 2H); MS (ESI): 640 [M+H]⁺.

### Example 43

### N-[3-(2-(Dimethylamino)-5-{2-[(6-{[2-(1-pyrrolidinyl)ethyl]oxy}-3-pyridinyl)amino]-4-pyrimidinyl}-1,3-thiazol-4-yl)phenyl]-2,6-difluorobenzamide

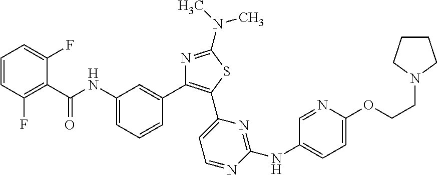

### Step A: 5-Nitro-2-{[2-(1-pyrrolidinyl)ethyl]oxy}pyridine

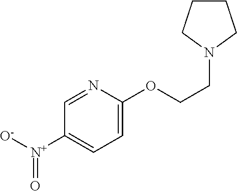

To a solution containing 4.1 mL (34.7 mmol) of 2-(1-pyrrolidinyl)ethanol and 50 mL of THF was added 1.7 g (4.1 mmol) of a 60% dispersion of sodium hydride in mineral oil. The reaction mixture was allowed to stir for 30 min and 5.0 g (18.9 mmol) of 2-chloro-5-nitropyridine was added slowly. The reaction mixture was allowed to stir for 3 h at rt, heated at 60° C. overnight, quenched by the addition of H₂O, and extracted with EtOAc. The combined organic layers were dried over MgSO₄ and the solvents were removed under reduced pressure. The residue was subjected to silica gel chromatography to give 3.7 g (45%) of 5-nitro-2-{[2-(1-pyrrolidinyl)ethyl]oxy}pyridine as a brown oil: ¹H-NMR (400 MHz, DMSO-d₆) δ 9.08 (d, J=2.93 Hz, 1H), 8.47 (dd, J=9.2 and 2.9 Hz, 1H), 7.04 (d, J=9.2 Hz, 1H), 4.49 (t, J=5.8 Hz, 2H), 2.80 (t, J=5.9 Hz, 2H), 2.45-2.53 (m, 4H), and 1.67 (ddd, J=6.8, 3.3, and 3.1 Hz, 4H).

### Step B: 6-{[2-(1-Pyrrolidinyl)ethyl]oxy}-3-pyridinamine hydrochloride

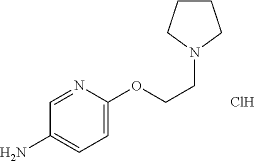

A mixture containing 3.7 g (15.5 mmol) of 5-nitro-2-{[2-(1-pyrrolidinyl)ethyl]oxy}pyridine, 0.3 g of 5% Platinum on carbon, and 30 mL of EtOH was subjected to a 50 psi H₂ atmosphere for 13 h. The reaction mixture was filtered through a pad of Celite, eluting with EtOH and EtOAc, and the solvent was removed under reduced pressure. The residue was taken up in EtOAc and 1.8 mL (3.7 mmol) of a 2.0 M solution of HCl in ether was added. The resulting mixture was filtered to give 2.8 g (74%) of 6-{[2-(1-pyrrolidinyl)ethyl]oxy}-3-pyridinamine hydrochloride as a white solid: ¹H-NMR (400 MHz, DMSO-d₆) δ 10.02 (brs, 1H), 7.62 (brs, 1H), 7.16 (d, J=8.1 Hz, 1H), 6.68 (d, J=8.8 Hz, 1H), 4.40-4.46 (m, 2H), 3.47-3.59 (m, 4H), 3.43 (brs, 2H), 3.08 (s, 2H), 1.99 (brs, 2H), and 1.87 (brs, 2H).

### Step C: N-[3-(2-(Dimethylamino)-5-{2-[(6-{[2-(1-pyrrolidinyl)ethyl]oxy}-3-pyridinyl)amino]-4-pyrimidinyl}-1,3-thiazol-4-yl)phenyl]-2,6-difluorobenzamide

To a suspension containing 0.1 g (0.21 mmol) of N-{3-[5-(2-chloro-4-pyrimidinyl)-2-(dimethylamino)-1,3-thiazol-4-yl]phenyl}-2,6-difluorobenzamide, prepared by a procedure analogous to Example 36, Step C, 0.06 g (0.23 mmol) of 6-{[2-(1-pyrrolidinyl)ethyl]oxy}-3-pyridinamine hydrochloride and 2 mL of i-PrOH was added 0.1 mL of a 4.0 M solution of HCl in dioxane. The reaction mixture was heated at 90° C. in a sealed tube for 3 days and the solvent was removed under reduced pressure. The residue was purified by HPLC to give 20 mg (15%) of N-[3-(2-(dimethylamino)-5-{2-[(6-{[2-(1-pyrrolidinyl)ethyl]oxy}-3-pyridinyl)amino]-4-pyrimidinyl}-1,3-thiazol-4-yl)phenyl]-2,6-difluorobenzamide as a yellow solid: ¹H-NMR (400 MHz, DMSO-d₆) δ 10.93 (s, 1H), 9.43 (s, 1H), 8.43 (d, J=2.8 Hz, 1H), 8.09 (d, J=5.3 Hz, 1H), 8.02 (dd, J=9.0 and 2.9 Hz, 1H), 7.85 (t, J=1.7 Hz, 1H), 7.80 (ddd, J=8.3, 2.1, and 1.0 Hz, 1H), 7.60 (ddd, J=15.1, 8.4, and 6.7 Hz, 1H), 7.47 (t, J=8.0 Hz, 1H), 7.26 (t, J=8.0 Hz, 3H), 6.78 (d, J=9.0 Hz, 1H), 6.28 (d, J=5.5 Hz, 1H), 4.30 (t, J=6.0 Hz, 2H), 3.14 (s, 6H), 2.76 (t, J=6.0 Hz, 2H), 2.47-2.52 (m, 4H), and 1.67 (ddd, J=6.8, 3.3, and 3.1 Hz, 4H); HRMS Calcd for: C₃₃H₃₃N₈O₂F₂S (M+H⁺): 643.2415. Found: 643.2420.

### Example 44

### 2,6-Difluoro-N-{3-[5-{2-[(2-methyl-1,2,3,4-tetrahydro-7-isoquinolinyl)amino]-4-pyrimidinyl}-2-(1-pyrrolidinyl)-1,3-thiazol-4-yl]phenyl}benzamide

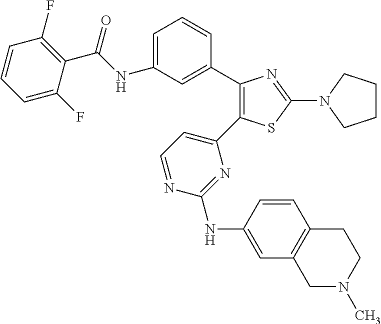

### Step A: 1-Pyrrolidinecarbothioamide

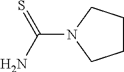

To obtain the title compound of Step A, pyrrolidine (1.5 g, 21 mmol) was placed in a round bottom flask under N₂ with stirring. THF (4 mL) was added followed by the drop-wise addition of 4N HCl in dioxane (5.3 mL, 21 mmol). Potassium thiocyanate (2.0 g, 21 mmol) was then added in one portion to the stirring solution of pyrrolidine hydrochloride. This mixture was then stirred at rt for 30 min followed by heating at 100° C. for 2 h. The reaction was then cooled to rt, MeOH (50 mL) was added, and solids that persisted were filtered away. Subsequent concentration of the MeOH/reaction solution yielded 3.0 g of the crude thiourea. ¹H-NMR (400 MHz, DMSO-d₆) δ 8.60 (brs, 2H), 3.07 (m, 4H), and 1.82 (m, 4H).

### Step B: N-{3-[5-(2-Chloro-4-pyrimidinyl)-2-(1-pyrrolidinyl)-1,3-thiazol-4-yl]phenyl}-2,6-difluorobenzamide

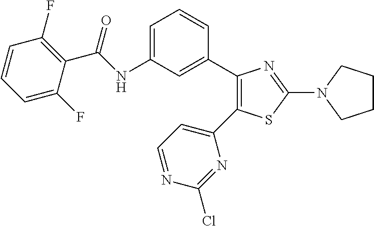

The title compound of Step B was prepared from N-{3-[(E)-2-(2-chloro-4-pyrimidinyl)-1-hydroxyethenyl]phenyl}-2,6-difluorobenzamide (1.48 g, 3.8 mmol), prepared by a procedure analogous to Example 1, Step C, NBS (0.68 g, 3.8 mmol), and 1-pyrrolidinecarbothioamide (1.0 g, 7.7 mmol), by a procedure analogous to Example 2, Step A. Yield 0.7 g (37%). ¹H-NMR (400 MHz, DMSO-d₆) δ 10.96 (s, 1H), 8.28 (d, J=5.6 Hz, 1H), 7.86 (s, 1H), 7.80 (d, J=8.4 Hz, 1H), 7.59 (m, 1H), 7.48 (t, J=8.0 Hz, 1H), 7.30-7.22 (m, 3H), 6.81 (d, J=5.4 Hz, 1H), 3.48 (m, 4H), and 2.01 (m, 4H); MS (ESI) m/z 498.1 (M+H)⁺.

### Step C: 2,6-Difluoro-N-{3-[5-{2-[(2-methyl-1,2,3,4-tetrahydro-7-isoquinolinyl)amino]-4-pyrimidinyl}-2-(1-pyrrolidinyl)-1,3-thiazol-4-yl]phenyl}benzamide

The title compound of Example 44 was prepared from N-{3-[5-(2-chloro-4-pyrimidinyl)-2-(1-pyrrolidinyl)-1,3-thiazol-4-yl]phenyl}-2,6-difluorobenzamide (0.1 g, 0.2 mmol) and 2-methyl-1,2,3,4-tetrahydro-7-isoquinolinamine (0.033 g, 0.2 mmol), by a procedure analogous to Example 1, Step D. Yield 0.052 g (42%). ¹H-NMR (400 MHz, DMSO-d₆) δ 10.92 (s, 1H), 9.38 (s, 1H), 8.07 (d, J=5.2 Hz, 1H), 7.84 (s, 1H), 7.79 (d, J=8.2 Hz, 1H), 7.67 (s, 1H), 7.58 (m, 1H), 7.46 (t, J=8.0 Hz, 1H), 7.34 (d, J=7.9 Hz, 1H), 7.28-7.21 (m, 3H), 6.98 (d, J=8.6 Hz, 1H), 6.24 (d, J=5.3 Hz, 1H), 3.48 (s, 2H), 3.47 (m, 4H), 2.74 (m, 2H), 2.57 (m, 2H), 2.32 (s, 3H), and 2.01 (m, 4H); MS (ESI) m/z 624.24 (M+H)⁺.

### Example 45

### N-{3-[5-{2-[(3-Chloro-4-{[2-(dimethylamino)ethyl]oxy}phenyl)amino]-4-pyrimidinyl}-2-(4-methyl-1-piperazinyl)-1,3-thiazol-4-yl]phenyl}-2,6-difluorobenzamide

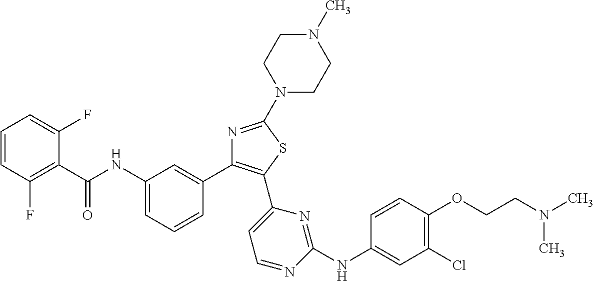

### Step A: 4-Methyl-1-piperazinecarbothioamide

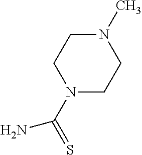

To a solution containing 1.0 mL (9.00 mmol) of 1-methylpiperazine and 5 mL of THF was added 2.25 mL (9.00 mmol) of a 4.0 M solution of HCl in dioxane. To this slurry was added a solution containing 0.80 g (8.19 mmol) of potassium thioisocyanate. The reaction mixture was heated at 50° C. for 13 h, and then cooled to it and MeOH was added. The reaction mixture was filtered and the solvents were removed from the filtrate to give 2.3 g of crude, wet thioamide, which was used without further purification: ¹H-NMR (400 MHz, DMSO-d₆) δ 8.56 (brs, 2H), 3.05 (t, J=5.10 Hz, 4H), 2.47-2.49 (m, 4H), and 2.22 (s, 3H).

### Step B: N-{3-[5-(2-Chloro-4-pyrimidinyl)-2-(4-methyl-1-piperazinyl)-1,3-thiazol-4-yl]phenyl}-2,6-difluorobenzamide

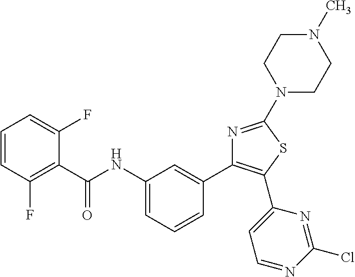

To a slurry containing 2.4 g (6.33 mmol) of N-{3-[5-(2-chloro-4-pyrimidinyl)-2-(4-methyl-1-piperazinyl)-1,3-thiazol-4-yl]phenyl}-2,6-difluorobenzamide, prepared by a procedure analogous to Example 1, Step C, and 30 mL of dioxane was added 1.13 g (6.33 mmol) of NBS. The reaction mixture was allowed to stir for 15 min and 2.3 g of crude 4-methyl-1-piperazinecarbothioamide was added. The reaction mixture was heated to 50° C. for 13 h, then quenched by the addition of H₂O and extracted with DCM. The combined organic layers were dried over MgSO₄, the solvents were removed under reduced pressure, and the residue was subjected to silica gel chromatography to give 2.0 g (60%) of the title compound of Step B as a yellow solid: ¹H-NMR (400 MHz, DMSO-d₆) δ 10.95 (s, 1H), 8.34 (d, J=5.5 Hz, 1H), 7.87 (s, 1 H), 7.81 (d, J=8.1 Hz, 1H), 7.54-7.64 (m, 1H), 7.49 (t, J=8.1 Hz, 1H), 7.23-7.31 (m, 3H), 6.90 (d, J=5.1 Hz, 1H), 3.57 (brs, 4H), 2.49 (brs, 4H) and; 2.27 (brs, 3H); ESIMS: 527318 (M+H⁺).

### Step C: N-{3-[5-{2-[(3-Chloro-4-{[2-(dimethylamino)ethyl]oxy}phenyl)amino]-4-pyrimidinyl}-2-(4-methyl-1-piperazinyl)-1,3-thiazol-4-yl]phenyl}-2,6-difluorobenzamide

To a slurry containing 125 mg (0.237 mmol) of N-{3-[5-(2-chloro-4-pyrimidinyl)-2-(4-methyl-1-piperazinyl)-1,3-thiazol-4-yl]phenyl}-2,6-difluorobenzamide and 2 mL of i-PrOH was added 59 mg (0.237 mmol) of 3-chloro-4-{[2-(dimethylamino)ethyl]oxy}phenyl amine and 0.1 mL of a 4.0 M solution of HCl in dioxane. The reaction mixture was heated at 90° C. for 72 h, then allowed to cool and quenched by the addition of TEA. The solvents were removed and the residue was purified by HPLC to give 87 mg (52%) of the title compound of Example 45 as an orange solid: ¹H-NMR (400 MHz, DMSO-d₆) δ 10.93 (s, 1H), 9.57 (s, 1H), 8.15 (d, J=5.3 Hz, 1H), 8.07 (d, J=2.6 Hz, 1H), 7.85-7.88 (m, 1H), 7.80 (d, J=8.1 Hz, 1H), 7.60 (t, J=8.1 Hz, 1H), 7.53 (dd, J=9.1, 2.84 Hz, 1H), 7.47 (t, J=8.4 Hz, 1H), 7.23-7.30 (m, 3H), 7.13 (d, J=9.5 Hz, 1H), 6.35 (d, J=5.3 Hz, 1H), 4.16 (t, J=5.8 Hz, 2H), 3.51-3.57 (m, 4H), 2.89 (brs, 2H), 2.47 (brs, 4H), 2.43 (s, 6H), and 2.25 (s, 3H); HRMS calcd for C₃₆H₂N₈O₂S: 705.2333 (M+H⁺). Found: 705.2332.

### Example 46

### 2,6-Difluoro-N-{3-[2-(4-methyl-1-piperazinyl)-5-(2-{[3-(1-pyrrolidinylmethyl)phenyl]amino}-4-pyrimidinyl)-1,3-thiazol-4-yl]phenyl}benzamide

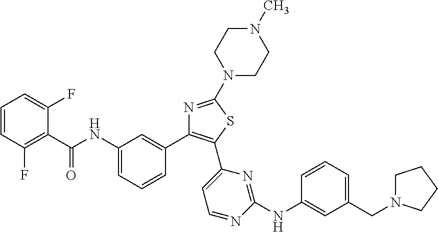

To a slurry containing 125 mg (0.237 mmol) of N-{3-[5-(2-chloro-4-pyrimidinyl)-2-(4-methyl-1-piperazinyl)-1,3-thiazol-4-yl]phenyl}-2,6-difluorobenzamide, prepared by a procedure analogous to Example 45, Step B, and 2 mL of i-PrOH was added 42 mg (0.237 mmol) 3-(1-pyrrolidinylmethyl)phenyl amine and 0.1 mL of a 4.0 M solution of HCl in dioxane. The reaction mixture was heated at 90° C. for 48 h, then allowed to cool and quenched by the addition of TEA. The solvents were removed and the residue was purified by HPLC to give 110 mg (70%) of the title compound of Example 46 as an orange solid: ¹H-NMR (400 MHz, DMSO-d₆) δ 10.96 (s, 1H), 9.52 (s, 1H), 8.14 (d, J=5.7 Hz, 1H), 7.84-7.92 (m, 2H), 7.77-7.81 (m, 1H), 7.49-7.60 (m, 2H), 7.43-7.47 (m, 1H), 7.19-7.29 (m, 4H), 6.90 (d, J=7.3 Hz, 1H), 6.36 (d, J=5.3 Hz, 1H), 3.50-3.59 (m, 6H), 2.41-2.48 (m, 8H), 2.24 (s, 3H), and 1.69 (s, 4H); HRMS calcd for C₃₆H₃₇F₂N₈OS: 667.2773 (M+H⁺). Found: 667.2770.

### Example 47

### N-[3-(2-(1-Azetidinyl)-5-{2-[(3-chloro-4-{[2-(1-pyrrolidinyl)ethyl]oxy}phenyl)amino]-4-pyrimidinyl}-1,3-thiazol-4-yl)phenyl]-2,6-difluorobenzamide

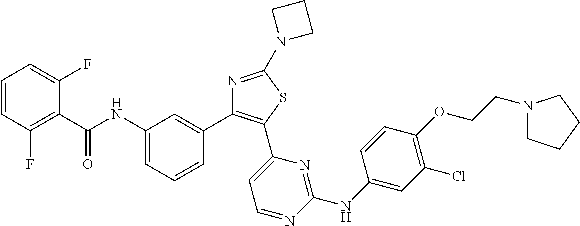

### Step A: 1-Azetidinecarbothioamide

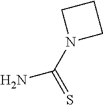

To a solution containing 0.6 mL (9.00 mmol) of azetidine and 5 mL of THF was added 2.25 mL (9.00 mmol) of a 4.0 M solution of HCl in dioxane. To this slurry was added a solution containing 0.80 g (8.19 mmol) of potassium thioisocyanate. The reaction mixture was heated at 50° C. for 13 h, then cooled to rt and MeOH was added. The reaction mixture was filtered and the solvents were removed from the filtrate to give crude, wet thioamide, which was used without further purification: ¹H-NMR (400 MHz, DMSO-d₆) δ 8.72 (brs, 1H), 3.87-3.95 (m, 4H), and 2.32-2.40 (m, 2H),

### Step B: N-{3-[2-(1-Azetidinyl)-5-(2-chloro-4-pyrimidinyl)-1,3-thiazol-4-yl]phenyl}-2,6-difluorobenzamide

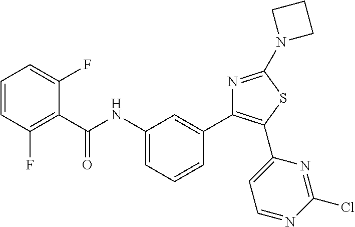

To a slurry containing 2.4 g (6.33 mmol) of N-{3-[5-(2-chloro-4-pyrimidinyl)-2-(4-methyl-1-piperazinyl)-1,3-thiazol-4-yl]phenyl}-2,6-difluorobenzamide, prepared by a procedure analogous to Example 1, Step C, and 20 mL of dioxane was added 1.13 g (6.33 mmol) of NBS. The reaction mixture was allowed to stir for 15 min and 2.3 g of crude 1-azetidinecarbothioamide was added. The reaction mixture was heated to 50° C. for 13 h, then quenched by the addition of water and extracted with DCM. The combined organic layers were washed with 10% aqueous HCl and dried over MgSO₄, The solvents were removed under reduced pressure and the residue was subjected to silica gel chromatography to give 2.0 g (60%) of the title compound as a yellow solid: ¹H-NMR (400 MHz, DMSO-d₆) δ 10.91 (s, 1H), 8.27 (d, J=5.7 Hz, 1H), 7.80 (s, 1H), 7.74 (d, J=8.2 Hz, 1H), 7.47-7.58 (m, 1H), 7.42 (t, J=7.9 Hz, 1H), 7.16-7.24 (m, 3H), 6.82 (d, J=5.7 Hz, 1H), 4.09 (t, J=7.5 Hz, 2H), and 2.43-2.45

### Step C: N-[3-(2-(1-Azetidinyl)-5-{2-[(3-chloro-4-{[2-(1-pyrrolidinyl)ethyl]oxy}phenyl)amino]-4-pyrimidinyl}-1,3-thiazol-4-yl)phenyl]-2,6-difluorobenzamide

To a slurry containing 125 mg (0.258 mmol) of N-{3-[2-(1-azetidinyl)-5-(2-chloro-4-pyrimidinyl)-1,3-thiazol-4-yl]phenyl}-2,6-difluorobenzamide and 2 mL of i-PrOH was added 71 mg (0.237 mmol) of 3-chloro-4-{[2-(1-pyrrolidinyl)ethyl]oxy}phenyl amine, prepared by a procedure analogous to Example 27, Step B, and 0.1 mL of a 4.0 M solution of HCl in dioxane. The reaction mixture was heated at 90° C. for 48 h, then allowed to cool and quenched by the addition of TEA. The solvents were removed and the residue was purified by HPLC and further purified by silica gel chromatography to give 39 mg (22%) of the title compound of Example 47 as a yellow solid: ¹H-NMR (400 MHz, DMSO-d₆) δ 11.08 (s, 1H), 9.50 (s, 1H), 8.00-8.04 (m, 2H), 7.76-7.88 (m, 2H), 7.51-7.62 (m, 3H), 7.19-7.29 (m, 3H), 7.10 (d, J=8.4 Hz, 1H), 5.80 (s, 1H), 4.10 (t, J=6.0 Hz, 2H), 3.50 (s, 2H), 2.77-2.82 (m, 2 H), 2.52-2.57 (m, 4H), 1.73-1.81 (m, 2H), 1.68 (s, 4H), and 1.23 (s, 2H); ESIMS: 686.16 (M−H⁻).

### Example 48

### N-[3-(2-Amino-5-{2-[(3-fluorophenyl)amino]-6-methyl-4-pyrimidinyl}-1,3-thiazol-4-yl)phenyl]-2,6-difluorobenzamide

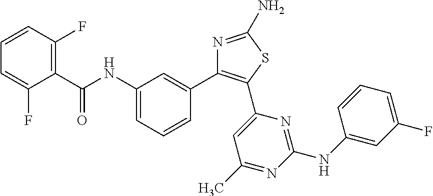

### Step A: N-{3-[(2-Chloro-6-methyl-4-pyrimidinyl)acetyl]phenyl}-2,6-difluorobenzamide

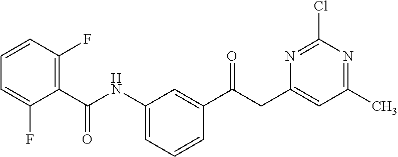

The title compound of Step A was prepared from ethyl 3-{[(2,6-difluorophenyl)carbonyl]amino}benzoate (0.392 g, 1.28 mmol), prepared by a procedure analogous to Example 1, Step B. 2-chloro-4,6-dimethylpyrimidine (0.2 g, 1.4 mmol) and LiHMDS (3.8 mL, 1 M in THF, 3.84 mmol), by a procedure analogous 10 Example 1, Step C. A mixture of the ketone and enolate form was isolated. Yield 0.512 g (100%). MS (ESI) m/z 402 (M+H)⁺.

### Step B: N-{3-[2-Amino-5-(2-chloro-6-methyl-4-pyrimidinyl)-1,3-thiazol-4-yl]phenyl}-2,6-difluorobenzamide

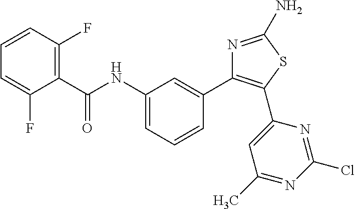

The title compound of Step B was prepared from N-{3-[(2-chloro-6-methyl-4-pyrimidinypacetyl]phenyl}-2,6-difluorobenzamide (0.2 g, 0.5 mmol), NBS (0.088 g, 0.5 mmol) and thiourea (0.4 g, 0.55 mmol) by a procedure analogous to Example 2, Step A. Yield 168 mg (74%). ¹H-NMR (400 MHz, DMSO-d₆) δ 10.88 (s, 1H), 7.93 (s, 1H), 7.87 (s, 1H), 7.69 (d, J=8.0 Hz, 1H), 7.58 (m, 1H), 7.43 (t, J=7.9 Hz, 1H), 7.29 (d, J=7.9 Hz, 1H), 7.24 (t, J=7.9 Hz, 2H), and 6.87 (s, 1H). MS (ESI) m/z 458 (M+H)⁺.

### Step C: N-[3-(2-Amino-5-{2-[(3-fluorophenyl)amino]-6-methyl-4-pyrimidinyl}-1,3-thiazol-4-yl)phenyl]-2,6-difluorobenzamide

The title compound of Example 48 was prepared from N-{3-[2-amino-5-(2-chloro-6-methyl-4-pyrimidinyl)-1,3-thiazol-4-yl]phenyl}-2,6-difluorobenzamide (0.08 g, 0.18 mmol) and 3-fluorophenyl amine (0.02 g, 0.18 mmol) by a procedure analogous to Example 1, Step D, and purified using silica gel chromatography and precipitation from DCM to yield 57 mg (58%). ¹H-NMR (300 MHz, DMSO-d₆) δ 10.87 (s, 1H), 9.72 (s, 1H), 7.94 (s, 1H), 7.82 (d, J=12.7 Hz, 1H), 7.70 (d, J=7.9 Hz, 1H), 7.64 (s, 2H), 7.58 (m, 1H), 7.42 (t, J=7.9 Hz, 2H), 7.31-7.20 (m, 4H), 6.71 (t, J=8.5 Hz, 1H), 6.40 (s, 1H), and 2.12 (s, 3H); MS (APCI) m/z 533.17 (M+H)⁺.

### Example 49

### N-[3-(2-Amino-5-{2-[(3-{[2-(dimethylamino)ethyl]oxy}phenyl)amino]-6-methyl-4-pyrimidinyl}-1,3-thiazol-4-yl)phenyl]-2,6-difluorobenzamide

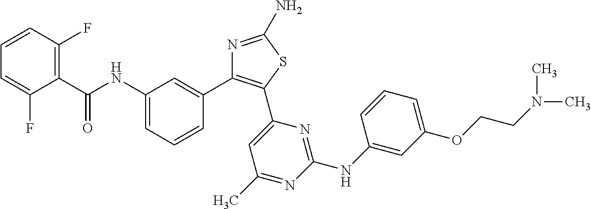

The title compound of Example 49 was prepared from N-{3-[2-amino-5-(2-chloro-6-methyl-4-pyrimidinyl)-1,3-thiazol-4-yl]phenyl}-2,6-difluorobenzamide (0.087 g, 0.19 mmol), prepared by a procedure analogous to Example 48, Step B, and 3-{[2-(dimethylamino)ethyl]oxy}phenyl amine (0.041 g, 0.19 mmol), by a procedure analogous to Example 1, Step D, and purified using silica gel chromatography and HPLC to yield 44 mg (38%). ¹H-NMR (400 MHz, DMSO-d₆) δ 10.88 (s, 1H), 9.42 (s, 1H), 7.94 (s, 1H), 7.70 (d, J=8.3 Hz, 1H), 7.62-7.53 (m, 3H), 7.45-7.38 (m, 2H), 7.35 (d, J=8.1 Hz, 1H), 7.31-7.21 (m, 3H), 7.11 (t, J=8.3 Hz, 1H), 6.49 (d, J=9.1 Hz, 1H), 6.36 (s, 1H), 4.04 (t, J=5.9 Hz, 2H), 2.62 (t, J=6.0 Hz, 2H), 2.21 (s, 6H), and 2.10 (s, 3H); HRMS C₃₁H₃₀N₇O₂F₂S (M+H)⁺ calcd 602.2150. found 602.2144.

### Example 50

### N-{3-[2-Amino-5-(2-{[3-fluoro-4-(methyloxy)phenyl]amino}-4-pyrimidinyl)-1,3-thiazol-4-yl]phenyl}-2,5-difluorobenzamide

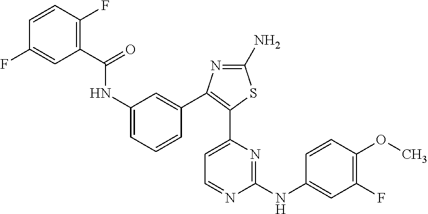

### Step A: Ethyl 3-{[(2,5-difluorophenyl)carbonyl]amino}benzoate

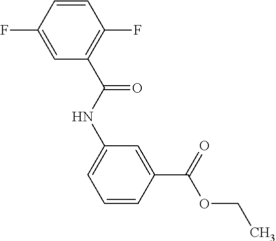

To a solution containing 14.8 g (89.6 mmol) of ethyl 3-aminobenzoate and 50 mL of DCM at 0° C. was added 11.6 mL (94.1 mmol) of 2,5-difluorobenzoyl chloride, followed by 13.7 mL (98.6 mmol) of TEA. The reaction mixture was allowed to stir for 1 h, then diluted with DCM and succesively washed with 10% aqueous HCl, 10% aqueous NaOH, and brine. The organic layers were dried over MgSO₄, filtered, and concentrated under reduced pressure to afford a quantitative yield of the title compound of Step A as an off-white solid: ¹H-NMR (d⁶-DMSO, 400 MHz) δ 10.7 (s, 1H), 8.36 (s, 1H), 7.95 (d, 1H, J=8.8 Hz), 7.70 (d, 1H, J=7.9 Hz), 7.54-7.58 (m, 1 H), 7.50 (dd, 1H, J=7.9 and 7.9 Hz), 7.39-7.47 (m, 2H), 4.31 (q, 2H, J=6.9 Hz), and 1.31 (t, 3H, J=6.9 Hz); ESIMS: 328.02 (M+Na⁺).

### Step B: N-{3-[(2-Chloro-4-pyrimidinypacetyl]phenyl}-2,5-difluorobenzamide

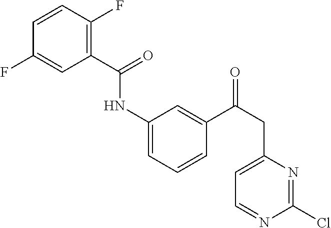

To a solution containing 16.0 g (52.8 mmol) of ethyl 3-{[(2,5-difluorophenyl)carbonyl]amino}benzoate and 100 mL of THF at 0° C. was added 170 mL (170 mmoL) of a 1.0 M solution of LHMDS in THF, followed immediately by a solution containing 6.8 g (52.8 mmol) of 2-chloro-4-methylpyrimidine and 10 mL of THF. The reaction mixture was allowed to stir for 1 h, then quenched by the addition of 6N HCl and extracted with EtOAc. The combined organic layers were dried over MgSO₄ and filtered, and the solvent was removed under reduced pressure. The residue was subjected to silica gel chromatography, eluting with a DCM/EtOAc mixture, to give 12.0 g (59%) of the title compound of Step B as an off white solid that exists as a mixture of ketone and enol tautomers: ¹H-NMR (d⁶-DMSO, 400 MHz) ketone: δ 10.72 (s, 1H), 8.74 (d, 1H, J=5.0 Hz), 8.32 (s, 1H), 8.0 (d, 1H, J=8.2Hz), 7.84 (m, 2H), and 4.66 (s, 2H); enol: 13.52 (s, 1H), 10.67 (s, 1H), 8.60 (d, 1 H, J=5.0 Hz), 7.82 (s, 1H), 7.65 (d, 1H, J=7.7 Hz), and 6.48 (s, 1H); shared: 7.54-7.66 (m, 2H) and 7.40-7.51 (m, 3H); ESIMS: 3.88.11 (M+H⁺).

### Step C: 2,5-Difluoro-N-{3-[(2-{[3-fluoro-4-(methyloxy)phenyl]amino}-4-pyrimidinyl)acetyl]phenyl}benzamide

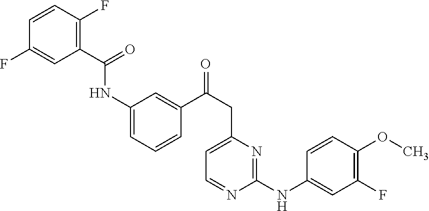

To a solution containing 2.4 g (6.2 mmol) of N-{3-[(2-chloro-4-pyrimidinypacetyl]phenyl}-2,5-difluorobenzamide and 60 mL of i-PrOH was added 0.92 g (6.5 mmol) of 3-fluoro-4-(methyloxy)phenyl amine and 0.1 mL of conc HCl. The reaction mixture was heated at 80° C. for 20 min, then diluted with an additional 60 mL of i-PrOH and heated at 90° C. for 13 h. The reaction mixture was allowed to cool to rt, then filtered. The filter cake was collected, slurried in DCM, and neutralized by the addition of aqueous NaHCO₃. The organic layer was separated and the aqueous layer was further extracted with DCM. The combined organic layers were dried over MgSO₄, filtered, and concentrated under recduced pressure. The residue was subjected to silica gel chromatography to give 1.76 g (59%) of the title compound of Step C as a yellow solid: ¹H-NMR (d⁶-DMSO, 400 MHz) δ 10.64 (s, 1H), 9.83 (s, 1H), 8.31 (d, 1H, J=5.3 Hz), 8.25 (s, 1H), 7.77 (d, 1H, J=8.4 Hz), 7.65-7.69 (m, 1H), 7.61 (d, 1H, J=7.7 Hz), 7.53-7.59 (m, 1H), 7.42-7.47 (m, 4H), 7.27 (d, 1H, J=9.2 Hz), 7.13 (dd, 1H, J=9.5 Hz and 9.5 Hz), 6.64 (d, 1H, J=5.3 Hz), 6.20 (s, 1), and 3.80 (s, 3H); ESIMS: 561.07 (M+H⁺).

### Step D: N-{3-[2-Amino-5-(2-{[3-fluoro-4-(methyloxy)phenyl]amino}-4-pyrimidinyl)-1,3-thiazol-4-yl]phenyl}-2,5-difluorobenzamide

To a solution containing 50 mg (0.10 mmol) of 2,5-difluoro-N-{3-[(2-{[3-fluoro-4-(methyloxy)phenyl]amino}-4-pyrimidinyl)acetyl]phenyl}benzamide and 5 mL of HOAc was added 5 μL (0.10 mmol) of bromine. The reaction mixture was allowed to stir for 20 h, then partitioned between water and EtOAc and neutralized by the careful addition of aqueous NaHCO₃. The neutralized mixture was extracted with EtOAc, dried over MgSO₄, filtered, and the solvent was removed under reduced pressure. The residue was taken up in 4 mL of dioxane, treated with 200 mg of thiourea and 200 mg of MgCO₃, and heated at 90° C. for 2 h. The reaction was allowed to cool to rt, diluted with EtOAc, and washed with brine. The organic layer was dried over MgSO₄ and filtered, and the solvent was removed under reduced pressure. The residue was pooled with another batch, also beginning with 50 mg of 2,5-difluoro-N-{3-[(2-{[3-fluoro-4-(methyloxy)phenyl]amino}-4-pyrimidinyl)acetyl]phenyl}benzamide and treated in the same way. The combined residue was subjected to silica gel chromatography to give 64 mg (78% combined yield) of the title compound of Example 50 as a light orange solid: ¹H-NMR (d⁶-DMSO, 400 MHz) δ 10.58 (s, 1H), 9.50 (s, 1H), 8.08 (d, 1H, J=5.3 Hz), 7.89 (s, 1H), 7.77 (d, 1H, J=8.3 Hz), 7.66 (s, 1H), 7.53 (s, 2H), 7.52-7.54 (m, 1H), 7.36-7.43 (m, 4H), 7.24 (d, 1H, J=7.7 Hz), 7.05 (dd, 1H, J=9.3 Hz), 6.32 (d, 1H, J=5.4 Hz), and 3.78 (s, 3H); HRMS calcd for C₂₇H₁₉F₃N₆O₂S: 548.1242. Found: 549.1321 (M+H⁺).

### Example 51

### 2,5-Difluoro-N-{3-[5-(2-{[3-fluoro-4-(methyloxy)phenyl]amino}-4-pyrimidinyl)-2-(methylamino)-1,3-thiazol-4-yl]phenyl}benzamide

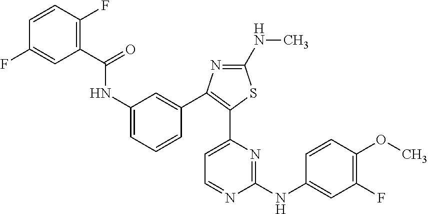

### Step A: N-{3-[Bromo(2-{[3-fluoro-4-(methyloxy)phenyl]amino}-4-pyrimidinyl)acetyl]phenyl}-2,5-difluorobenzamide

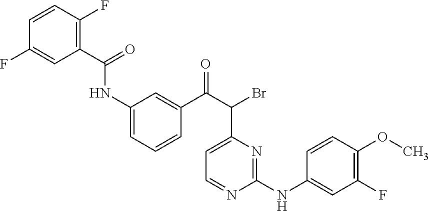

To a solution containing 357 mg (0.73 mmol) of 2,5-difluoro-N-{3-[(E)-2-(2-{[3-fluoro-4-(methyloxy)phenyl]amino}-4-pyrimidinyl)-1-hydroxyethenyl]phenyl}-benzamide, prepared by a procedure analogous to Example 50, Step B, and 10 mL of HOAc was added 37 μL (0.73 mmol) of bromine dropwise. The reaction mixture was allowed to stir for 20 min, then partitioned between water and EtOAc and neutralized by the careful addition of aqueous NaHCO₃. The neutralized mixture was extracted with EtOAc, dried over MgSO₄, filtered, and the solvent was removed under reduced pressure to give the title compound of Step A as a thick, brown oil that was used without further purification: ESIMS: 571.10 (M⁺).

### Step B: 2,5-Difluoro-N-{3-[5-(2-{[3-fluoro-4-(methyloxy)phenyl]amino}-4-pyrimidinyl)-2-(methylamino)-1,3-thiazol-4-yl]phenyl}benzamide

To a solution containing 68 mg (0.12 mmol) of N-{3-[bromo(2-{[3-fluoro-4-(methyloxy)phenyl]amino}-4-pyrimidinypacetyl]phenyl}-2,5-difluorobenzamide and 2 mL of dioxane was added 100 mg of N-methylthiourea and 100 mg of MgCO₃. The reaction was heated at 90° C. for 4 h, then cooled to rt and concentrated under reduced pressure. The residue was subjected to silica gel chromatography and HPLC purification to give 17 mg (25%) of the title compound of Example 51 as a light orange solid: ¹H-NMR (d⁶-DMSO, 400 MHz) δ 10.58 (s, 1H), 9.48 (s, 1H), 8.18-8.20 (m, 1H), 8.06 (d, 1H, J=5.5 Hz), 7.85 (s, 1H), 7.75-7.79 (m, 2H), 7.49-7.53 (m, 1H), 7.34-7.44 (m, 4H), 7.22 (d, 1H, J=7.9 Hz), 7.04 (dd, 1H, J=9.3 Hz), 6.27 (d, 1H, J=5.5 Hz), 3.76 (s, 3H), and 2.86 (d, 3H, J=4.6 Hz); ESIMS: 563.20 (M+H⁺).

### Example 52

### 2,5-Difluoro-N-[3-(5-(2-{[3-fluoro-4-(methyloxy)phenyl]amino}-4-pyrimidinyl)-2-{[2-(methyloxy)ethyl]amino}-1,3-thiazol-4-yl)phenyl]benzamide

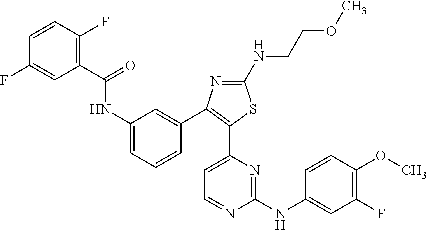

To a solution containing 68 mg (0.12 mmol) of N-{3-[bromo(2-{[3-fluoro-4-(methyloxy)phenyl]amino}-4-pyrimidinypacetyl]phenyl}-2,5-difluorobenzamide, prepared by a procedure analogous to Example 51, Step A, and 2 mL of dioxane was added 100 mg of N-[2-(methyloxy)ethyl]thiourea and 100 mg of MgCO₃. The reaction was heated at 90° C. for 4 h, then cooled to it and concentrated under reduced pressure. The residue was subjected to silica gel chromatography and HPLC purification to give 23 mg (32%) of the title compound of Example 52 as a yellow solid: ¹H-NMR (d⁶-DMSO, 400 MHz) δ 10.58 (s, 1H), 9.48 (s, 1H), 8.33-8.36 (m, 1H), 8.07 (d, 1H, J=5.3 Hz), 7.84 (s, 1H), 7.75-7.78 (m, 2H), 7.50-7.53 (m, 1H), 7.34-7.43 (m, 4H), 7.22 (d, 1H, J=7.5 Hz), 7.03 (dd, 1H, J=9.5 Hz), 6.28 (d, 1H, J=5.3 Hz), 3.76 (s, 3H), 3.44-3.48 (m, 4H), and 3.26 (s, 3H); HRMS calcd for C₃₀H₂₃F₃N₆O₃S: 606.1661. Found: 607.1739 (M+H⁺).

### Example 53

### 2,5-Difluoro-N-[3-(5-(2-{[3-fluoro-4-(methyloxy)phenyl]amino}-4-pyrimidinyl)-2-{[2-(4-morpholinyl)ethyl]amino}-1,3-thiazol-4-yl)phenyl]benzamide

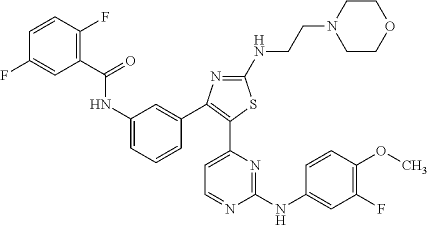

To a solution containing 68 mg (0.12 mmol) of N-{3-[bromo(2-{[3-fluoro-4-(methyloxy)phenyl]amino}-4-pyrimidinyl)acetyl]phenyl}-2,5-difluorobenzamide, prepared by a procedure analogous to Example 51, Step A, and 2 mL of dioxane was added 100 mg of N-[2-(4-morpholinyl)ethyl]thiourea and 100 mg of MgCO₃. The reaction was heated at 90° C. for 4 h, then cooled to rt and concentrated under reduced pressure. The residue was subjected to silica gel chromatography, eluting with DCM/MeOH to give 32 mg (41%) of the title compound of Example 53 as a yellow solid: ¹H-NMR (d⁶-DMSO, 400 MHz) δ 10.58 (s, 1H), 9.49 (s, 1H), 8.21-8.23 (m, 1H), 8.06 (d, 1H, J=5.4 Hz), 7.85 (s, 1H), 7.76-7.79 (m, 2H), 7.50-7.53 (m, 1H), 7.34-7.43 (m, 4H), 7.22 (d, 1H, J=7.7 Hz), 7.04 (dd, 1H, J=9.5 Hz), 6.29 (d, 1H, J=5.4 Hz), 3.77 (s, 3H), 3.53-3.56 (m, 4H), 3.38-3.40 (m, 2H), 2.48-2.51 (m, 2H), and 2.29-2.48 (m, 4H); HRMS calcd for C₃₃H₃₀F₃N₇O₃S: 661.2083. Found: 662.2161 (M+H⁺).

### Example 54

### N-{3-[2-Amino-5-(2-{[3-(1,3-oxazol-5-yl)phenyl]amino}-4-pyrimidinyl)-1,3-thiazol-4-yl]phenyl}-2,5-difluoro-N-methylbenzamide

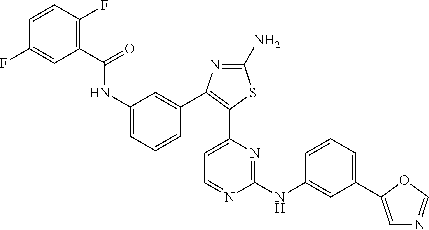

### Step A: 2,5-Difluoro-N-methyl-N-{3-[(2-{[3-(1,3-oxazol-5-yl)phenyl]amino}-4-pyrimidinyl)acetyl]phenyl}benzamide

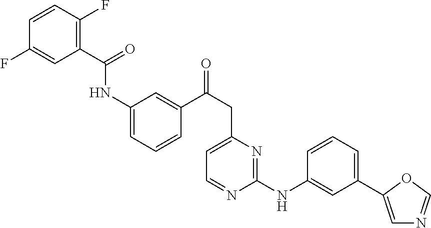

To a solution containing 250 mg (0.64 mmol) of N-{3-[(2-chloro-4-pyrimidinyl)acetyl]phenyl}-2,5-difluorobenzamide, prepared by a procedure analogous to Example 50, Step B, 1.5 mL of i-PrOH, and 0.5 mL of DMA was added 103 mg (0.64 mmol) 3-(1,3-oxazol-5-yl)phenyl amine and 1 drop of conc HCl. The reaction mixture was heated at 70° C. for 12 h, diluted with H₂O, and filtered to give the title compound of Step A as a yellow solid, which was used without further purification: ESIMS: 512.20 (M+H⁺).

### Step B: N-{3-[2-Amino-5-(2-{[3-(1,3-oxazol-5-yl)phenyl]amino}-4-pyrimidinyl)-1,3-thiazol-4-yl]phenyl}-2,5-difluoro-N-methylbenzamide

To a slurry containing the yellow solid from Step A, and 5 mL of HOAc was added 33 μL (0.64 mmol) of bromine. The reaction mixture was allowed to stir at rt for 1 h, then poured into H₂O, neutralized by the addition of K₂CO₃, and extracted with DCM. The combined organic layers were dried over MgSO₄ and filtered, and the solvents were removed under reduced pressure. The residue was dissolved in 5 mL of dioxane and 49 mg (0.64 mmol) of thiourea and 54 mg (0.64 mmol) of MgCO₃ were added. The reaction mixture was heated at 50° C. for 13 h, then diluted with H₂O and extracted with DCM. The combined organic layers were dried over MgSO₄ and filtered, and the solvents were removed under reduced pressure. The residue was subjected to silica gel purification and HPLC purification to give 17 mg (5%) of the title compound of Example 54 as a yellow solid: ¹H-NMR (d⁶-DMSO, 400 MHz) δ 10.56 (s, 1H), 9.64 (s, 1H), 8.44 (s, 1H), 8.11 (d, 1H, J=5.3 Hz), 7.89 (s, 1H), 7.76 (d, 1H, J=8.5 Hz), 7.70 (d, 1H, J=8.1 Hz), 7.65 (s, 2H), 7.59 (s, 1H), 7.50-7.54 (m, 2H), 7.39-7.46 (m, 3H), 7.28-7.35 (m, 2H), 7.24 (d, 1H, J=7.5 Hz), and 6.35 (d, 1H, J=5.4 Hz); HRMS calcd for C₂₃H₁₃N₇OF₂S: 567.1289. Found: 568.1367 (M+H⁺).

### Example 55

### N-[3-(2-Amino-5-{2-[(4-{[3-(dimethylamino)propyl]amino}-3-fluorophenyl)amino]-4-pyrimidinyl}-1,3-thiazol-4-yl)phenyl]-2,5-difluoro-N-methylbenzamide

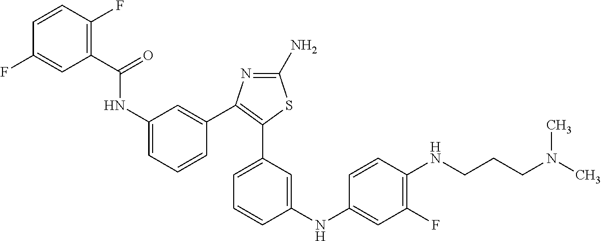

### Step A: N-[3-({2-[(4-{[3-(Dimethylamino)propyl]amino}-3-fluorophenyl)amino]-4-pyrimidinyl}acetyl)phenyl]-2,5-difluoro-N-methylbenzamide

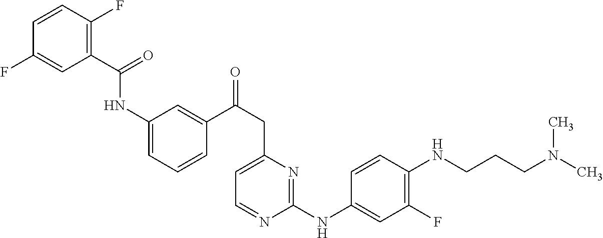

To a solution containing 300 mg (0.68 mmol) of N-{3-[(2-chloro-4-pyrimidinyl)acetyl]phenyl}-2,5-difluorobenzamide, prepared by a procedure analogous to Example 50, Step B, 1.5 mL of i-PrOH and 0.5 mL of DMA was added 167 mg (0.68 mmol) of N¹-[3-(dimethylamino)propyl]-2-fluoro-1,4-benzenediamine and 1 drop of conc HCl. The reaction mixture was heated at 70° C. for 48 h, and then partitioned between DCM and H₂O. The H₂O later was further extracted with DCM and the combined organics were dried over MgSO₄, filtered, and concentrated under reduced pressure. The residue was subjected to silica gel chromatography, eluting with a mixture of EtOAc/MeOH/NH₄OH to give 286 mg (75%) of the title compound of Step A as an orange foam, a mixture of keto and enol tautomers: ESIMS: 563.30

### Step B: N-[3-(2-Amino-5-{2-[(4-{[3-(dimethylamino)propyl]amino}-3-fluorophenyl)amino]-4-pyrimidinyl}-1,3-thiazol-4-yl)phenyl]-2,5-difluoro-N-methylbenzamide

To a solution containing 280 mg (0.50 mmol) of N-[3-({2-[(4-{[3-(dimethylamino)propyl]amino}-3-fluorophenyl)amino]-4-pyrimidinyl}acetyl)phenyl]-2,5-difluoro-N-methylbenzamide and 5 mL of HOAc was added 26 μL (0.50 mmol) of bromine dropwise. The reaction mixture was allowed to stir at rt for 2 h, then poured into water, neutralized by the addition of 10% aqueous NaOH, and extracted with DCM. The combined organic layers were dried over MgSO₄ and filtered, and the solvents were removed under reduced pressure. The residue was taken up in 5 mL of dioxane and 38 mg (0.50 mmol) of thiourea and 42 mg (0.50 mmol) of MgCO₃ was added. The reaction mixture was heated at 50° C. for 4 h, then allowed to cool to rt, diluted with H₂O, and extracted with DCM. The combined organic layers were dried over MgSO₄ and filtered, and the solvents were removed under reduced pressure. The residue was subjected to silica gel purification and HPLC purification to give 28 mg (9%) of the title compound of Example 55 as an orange solid: ¹H-NMR (d⁶-DMSO, 400 MHz) δ 10.57 (s, 1H), 9.24 (s, 1H), 8.16 (s, 1H), 8.03 (d, 1H, J=5.3 Hz), 7.88 (s, 1H), 7.76 (d, 1H, J=9.7 Hz), 7.62 (s, 2H), 7.50-7.58 (m, 1H), 7.39-7.43 (m, 2H), 7.21-7.25 (m, 2H), 6.61 (t, 1H, J=9.1 Hz), 6.25 (d, 1H, J=5.3 Hz), 3.06 (t, 2H, J=6.8 Hz), 2.52 (s, 1H), 2.30-2.36 (m, 2H), 2.17 (s, 6H), and 1.67-1.72 (m, 2H); HRMS calcd for C₃₁H₂₉N₈OF₃S: 618.2137. Found: 619.2220 (M+H⁺).

### Example 56

### N-[3-(2-Amino-5-{2-[(3-chloro-4-{[2-(dimethylamino)ethyl]oxy}phenyl)amino]-4-pyrimidinyl}-1,3-thiazol-4-yl)phenyl]-2,5-difluoro-N-methylbenzamide

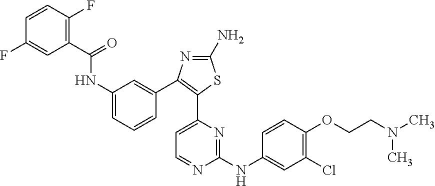

### Step A: N-[3-({2-[(3-Chloro-4-{[2-(dimethylamino)ethyl]oxy}phenyl)amino]-4-pyrimidinyl}acetyl)phenyl]-2,5-difluoro-N-methylbenzamide

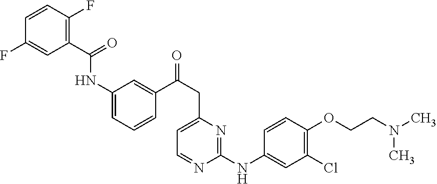

To a slurry containing 250 mg (0.64 mmol) of N-{3-[(2-chloro-4-pyrimidinyl)acetyl]phenyl}-2,5-difluorobenzamide, prepared by a procedure analogous to Example 50, Step A, 1 mL of i-PrOH, and 0.5 mL of DMA, was added 180 mg (0.64 mmol) of 3-chloro-4-{[2-(dimethylamino)ethyl]oxy}phenyl amine and 1 drop of conc HCl. The reaction mixture was heated at 70° C. for 48 h, then allowed to cool to it and diluted in H₂O. The reaction mixture was neutralized by the addition of 10% aqueous NaOH and extracted with DCM. The combined organic layers were dried over MgSO₄ and filtered and the solvent was removed under reduced pressure. The residue was subjected to silica gel chromatography, eluting with an EtOAc/MeOH/NH₄OH mixture to give 274 mg (75%) of the title compound of Step A as an orange oil that exists as a mixture of ketone and enol tautomers: ESIMS: 566.30 (M+H⁺).

### Step B: N-[3-(2-Amino-5-{2-[(3-chloro-4-{[2-(dimethylamino)ethyl]oxy}phenyl)amino]-4-pyrimidinyl}-1,3-thiazol-4-yl)phenyl]-2,5-difluoro-N-methylbenzamide

To a slurry containing 256 mg (0.45 mmol) of N-[3-({2-[(3-chloro-4-{[2-(dimethylamino)ethyl]oxy}phenypamino]-4-pyrimidinyl}acetyl)phenyl]-2,5-difluoro-N-methylbenzamide and 5 mL of DCM was added 84 mg (0.48 mmol) of NBS. The reaction mixture was allowed to stir at rt for 2 h and the solvent was removed under reduced pressure. The residue was taken up in dioxane and 34 mg (0.45 mmol) of thiourea and 38 mg (0.45 mmol) of MgCO₃ was added. The reaction mixture was heated to 50° C. for 1 h and allowed to cool to rt, diluted in H₂O, and extracted with DCM. The combined organic layers were dried over MgSO₄ and filtered, and the residue was subjected to silica gel chromatography and HPLC purification to give 71 mg (25%) of the title compound of Example 56 as an orange solid: ¹H-NMR (d⁶-DMSO, 400 MHz) δ 10.57 (s, 1H), 9.48 (s, 1H), 8.13 (s, 1H), 8.09 (d, 1H, J=5.3 Hz), 7.93 (d, 1H, J=2.5 Hz), 7.89 (s, 1H), 7.76 (d, 1H, J=8.2 Hz), 7.65 (s, 2H), 7.52-7.57 (m, 2H), 7.39-7.43 (m, 2H), 7.23 (d, 1H, J=7.5 Hz), 7.06 (d, 1H, J=8.9 Hz), 6.32 (d, 1H, J=5.3 Hz), 4.08 (t, 2H, J=5.6 Hz), 2.68 (t, 2H, J=5.7 Hz), and 2.26 (s, 6H); HRMS calcd for C₃₀H₂₆N₇O₂F₂CIS: 621.1525. Found: 622.1604 (M+H⁺).

### Example 57

### N-(3-{2-Amino-5-[2-(1,2,3,4-tetrahydro-7-isoquinolinylamino)-4-pyrimidinyl]-1,3-thiazol-4-yl}phenyl)-2,5-difluoro-N-methylbenzamide: formic acid

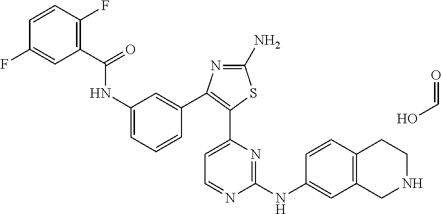

### Step A: 2,5-Difluoro-N-methyl-N-{3-[(2-{[2-(trifluoroacetyl)-1,2,3,4-tetrahydro-7-isoquinolinyl]amino}-4-pyrimidinyl)acetyl]phenyl}benzamide

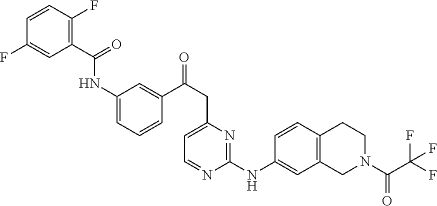

To a solution containing 250 mg (0.64 mmol) of N-{3-[(2-chloro-4-pyrimidinypacetyl]phenyl}-2,5-difluorobenzamide, prepared by a procedure analogous to Example 50, Step B, 1.5 mL of i-PrOH, and 0.5 mL of DMA, was added 180 mg (0.68 mmol) of 2-(trifluoroacetyl)-1,2,3,4-tetrahydro-7-isoquinolinamine and 1 drop of conc HCl. The reaction mixture was heated at 70° C. for 48 h, then diluted with H₂O. The reaction mixture was neutralized by the addition of 10% aqueous NaOH and extracted with DCM. The combined organic layers were dried over MgSO₄, filtered, and concentrated under reduced pressure. The residue was subjected to silica gel chromatography, eluting with a mixture of EtOAc/MeOH/NH₄OH to give 274 mg (72%) of the title compound of Step A as an orange foam, a mixture of keto and enol tautomers: ESIMS: 596.30 (M+H⁺).

### Step B: N-(3-{2-Amino-5-[2-(1,2,3,4-tetrahydro-7-isoquinolinylamino)-4-pyrimidinyl]-1,3-thiazol-4-yl}phenyl)-2,5-difluoro-N-methylbenzamide: formic acid

To a slurry containing 274 mg (0.46 mmol) of 2,5-difluoro-N-methyl-N-{3-[(2-{[2-(trifluoroacetyl)-1,2,3,4-tetrahydro-7-isoquinolinyl]amino}-4-pyrimidinyl)acetyl]phenyl}benzamide and 5 mL of DCM was added 86 mg (0.48 mmol) of NBS. The reaction mixture was allowed to stir at rt for 2 h and the solvent was removed under reduced pressure. The residue was taken up in dioxane and 34 mg (0.45 mmol) of thiourea and 38 mg (0.45 mmol) of MgCO₃ was added. The reaction mixture was heated to 50° C. for 1 h, allowed to cool to it, diluted in H₂O, and extracted with DCM. The combined organic layers were dried over MgSO₄ and filtered, and the residue was subjected to silica gel chromatography and HPLC purification to give 18 mg (7%) of the title compound of Example 57 as a yellow solid: ¹H-NMR (d⁶-DMSO, 400 MHz) δ 10.58 (s, 1H), 9.39 (s, 1H), 8.20 (s, 1H), 8.07 (d, 1H, J=5.3 Hz), 7.90 (s, 1H), 7.76 (d, 1H, J=7.5 Hz), 7.64 (s, 2H), 7.50-7.55 (m, 2H), 7.39-7.43 (m, 3H), 7.23 (d, 1H, J=7.5 Hz), 6.98 (d, 1H, J=8.4 Hz), 6.31 (d, 1H, J=5.3 Hz), 3.96 (s, 2H), 3.06 (t, 2H, J=6.3 Hz), and 2.70 (t, 2H, J=6.3 Hz); HRMS calcd for C₂₃H₂₃N₇OF₂S: 555.1653. Found: 556.1731 (M+H⁺).

### Example 58

### N-[3-(2-Amino-5-{2-[(3-fluoro-4-{[2-(methyloxy)ethy]oxy}phenyl)amino]-4-pyrimidinyl}-1,3-thiazol-4-yl)phenyl]-2,5-difluorobenzamide

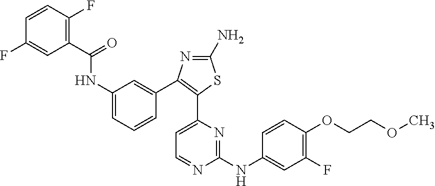

### Step A: N-{3-[2-Amino-5-(2-chloro-4-pyrimidinyl)-1,3-thiazol-4-yl]phenyl}-2,5-difluorobenzamide

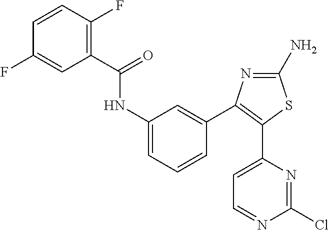

To a slurry containing 3.69 g (9.5 mmol) of N-{3-[bromo(2-{[3-fluoro-4-(methyloxy)phenyl]amino}-4-pyrimidinyl)acetyl]phenyl}-2,5-difluorobenzamide, prepared by a procedure analogous to Example 50, Step B, and 50 mL of DCM, was added 1.7 g (9.5 mmol) of NBS. The reaction mixture was allowed to stir at it for 15 min, until all reagents fully dissolved, and the solvent was removed under reduced pressure. The residue was taken up in 50 mL of dioxane and 0.80 g (10.5 mmol) of thiourea was added. The reaction mixture was allowed to stir for 13 h at rt, then diluted in EtOAc and washed with 10% aqueous HCl and brine. The combined organic layers were dried over MgSO₄, filtered, and the solvent was removed under reduced pressure. The residue was subjected to silica gel chromatography, eluting with a mixture of MeOH/DCM, to give 3.76 g (90%) of the title compound of Step A as a yellow solid: ¹H-NMR (d⁶-DMSO, 400 MHz) δ 10.60 (s, 1H), 8.28 (d, 1H, J=5.5 Hz), 7.89 (s, 1H), 7.78 (d, 1H, J=8.3 Hz), 7.49-7.53 (m, 1H), 7.37-7.46 (m, 3 H), 7.25 (d, 1H, J=7.5 Hz), 6.86 (d, 1H, J=5.5 Hz), and 5.83 (brs, 2H); ESIMS: 444.05 (M+H⁺).

### Step B: N-[3-(2-Amino-5-{2-[(3-fluoro-4-{[2-(methyloxy)ethyl]oxy}phenypamino]-4-pyrimidinyl}-1,3-thiazol-4-yl)phenyl]-2,5-difluorobenzamide

To a solution containing 100 mg (0.23 mmol) of N-{3-[2-amino-5-(2-chloro-4-pyrimidinyl)-1,3-thiazol-4-yl]phenyl}-2,5-difluorobenzamide and 1 mL of i-PrOH was added 50 mg (0.23 mmol) of 3-fluoro-4-{[2-(methyloxy)ethyl]oxy}phenyl amine and 1 drop of conc HCl. The reaction mixture was heated to 75° C. for 36 h, cooled to rt, and partitioned between H₂O and EtOAc. The organic layers were dried over MgSO₄, filtered, and the solvent was removed under reduced pressure. The residue was subjected to silica gel chromatography, eluting with a mixture of MeOH/DCM, to give 27 mg (20%) of the title compound of Example 58 as a yellow solid: ¹H-NMR (d⁶-DMSO, 400 MHz) δ 10.55 (s, 1H), 9.48 (s, 1H), 8.07 (d, 1H, J=5.5 Hz), 7.87 (s, 1H), 7.73-77.76 (m, 2H), 7.64 (s, 2H), 7.49-7.51 (m, 1H), 7.38-7.42 (m, 3H), 7.33 (d, 1H, J=9.1 Hz), 7.22 (d, 1H, J=7.5 Hz), 7.03 (dd, 1H, J=9.5 and 9.5 Hz), 6.31 (d, 1H, J=5.3 Hz), 4.09 (t, 2H, J=4.4 Hz), 3.62 (t, 2H, J=4.4 Hz), and 3.36 (s, 3H); ESIMS: 593.30 (M+H⁺).

### Example 59

### N-(3-{2-Amino-5-[2-(1,2,3,4-tetrahydro-7-isoquinolinylamino)-4-pyrimidinyl]-1,3-thiazol-4-yl}phenyl)-N-methylcyclohexanecarboxamide

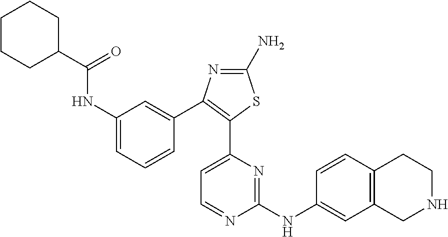

### Step A: Ethyl 3-[(cyclohexylcarbonyl)amino]benzoate

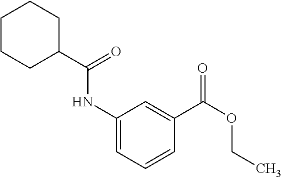

To a solution containing 7.5 g (45.9 mmol of ethyl 3-aminobenzoate and 250 mL of DCM at 0° C. was added 6.3 mL (48.1 mmol) of cyclohexanecarbonyl chloride, followed by 8.04 mL (57.7 mmol) of TEA. The reaction mixture was allowed to stir for 1 h, then diluted with DCM and successively washed with 10% aqueous HCl, 10% aqueous NaOH, and brine. The organic layers were dried over MgSO₄, filtered, and concentrated under reduced pressure to afford a quantitative yield of the title compound of Step A as an off-white solid: ¹H-NMR (d⁶-DMSO, 400 MHz) δ 9.99 (s, 1H), 8.23 (s, 1H), 7.82 (d, 1H, J=7.9 Hz), 7.57 (d, 1H, J=7.5 Hz), 7.39 (t, 1H, J=7.8 Hz), 2.26-2.32 (m, 1H), 4.28 (q, 2H, J=7.1 Hz), 1.71-1.79 (m, 4H), 1.82 (d, 1H, J=11.0 Hz), and 1.10-1.42 (m, 8H); ESIMS: 276.30 (M+H⁺).

### Step B: N-{3-[(2-Chloro-4-pyrimidinyl)acetyl]phenyl}-N-methylcyclohexanecarboxamide

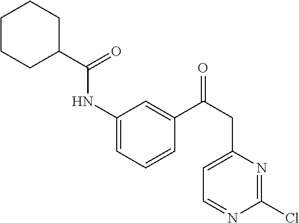

To a solution containing 4.5 g (16.3 mmol) of ethyl 3-[(cyclohexylcarbonyl)-amino]benzoate was added 10.2 mL (16.3 mmol) of a 1.6 M solution of 4-0 chloropyrimidine in THF. The solution was cooled to 0° C. and 49 mL (49.0 mmol) of a 1.0 M solution of LHMDS in THF was added slowly. The reaction mixture was allowed to stir at 0° C. for 2 h, at which time an additional 5 mL (8.0 mmol) of a 1.6 M solution of 4-methyl-2-chloropyrimidine in THF and an additional 10 mL (10 mmol) of a 1.0 M solution of LHMDS in THF was added. The reaction mixture was allowed to warm to rt and stirred for an additional 12 h, then quenched by the addition of aqueous NH₄Cl and extracted with DCM. The combined organic layers were dried over MgSO₄ and filtered, and the solvent was removed under reduced pressure. The residue was subjected to silica gel chromatography, eluting with an EtOAc/hexane mixture, to give 2.96 g (51%) of the title compound of Step B as an off white solid: ¹H-NMR (CDCl₃, 400 MHz) δ 13.73 (s, 1H), 8.39 (d, 1H, J=5.5 Hz), 8.1 (s, 1H), 7.59 (d, 1H, J=7.8 Hz), 7.40 (t, 1H, J=7.9 Hz), 7.00 (s, 1H), 6.91 (d, 1H, J=5.5 Hz), 6.09 (s, 1H), 2.23-2.29 (m, 1H), 1.95-2.00 (m, 2H), 1.85-1.88 (m, 2H), 1.73-1.75 (m, 1H), 1.53-1.61 (m, 2H), and 1.25-1.43 (m, 4H); ESIMS: 358.20 (M+H⁺).

### Step C: N-Methyl-N-{3-[(2-{[2-(trifluoroacetyl)-1,2,3,4-tetrahydro-7-isoquinolinyl]amino}-4-pyrimidinyl)acetyl]phenyl}cyclohexanecarboxamide

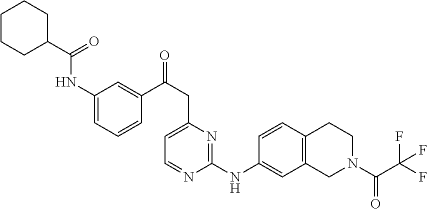

To a slurry containing 320 mg (0.89 mmol) of N-{3-[(2-chloro-4-pyrimidinyl)acetyl]phenyl}-N-methylcyclohexanecarboxamide, and 1 mL of i-PrOH was added 250 mg (0.89 mmol) of 2-(trifluoroacetyl)-1,2,3,4-tetrahydro-7-isoquinolinamine and 1 drop of conc HCl. The reaction mixture was heated at 70° C. for 48 h, then cooled to rt and treated with water and 10% aqueous NaOH. The resulting precipitate was filtered to collect 500 mg (100%) of the title compound as a yellow solid that was used without further purification: ESIMS: 566.30 (M+H⁺).

### Step D: N-{3-[2-Amino-5-(2-{[2-(trifluoroacetyl)-1,2,3,4-tetrahydro-7-isoquinolinyl]amino}-4-pyrimidinyl)-1,3-thiazol-4-yl]phenyl}-N-methylcyclohexanecarboxamide

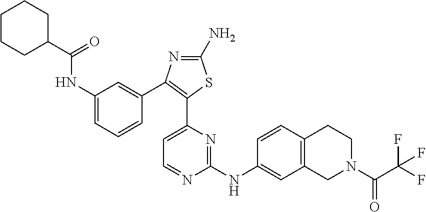

To a solution containing 500 mg (0.89 mmol) of N-methyl-N-{3-[(2-{[2-(trifluoroacetyl)-1,2,3,4-tetrahydro-7-isoquinolinyl]amino}-4-pyrimidinyl)acetyl]phenyl}cyclohexanecarboxamide, 1 mL of DCM, and 5 mL of HOAc, was added 45 μL (0.89 mmol) of bromine. The reaction mixture was allowed to stir at rt for 2 h, then diluted in H₂O and quenched by the addition of aqueous NaHCO₃. The mixture was extracted with DCM and the combined organic layers were dried over MgSO₄ and filtered. The solvent was removed under reduced pressure and the residue was subjected to silica gel chromatography, eluting with an EtOAc/hexanes mixture, to give 250 mg (45%) of the title compound of Step D as a yellow solid: ¹H-NMR (d⁶-DMSO, 400 MHz) δ 9.87 (s, 1H), 9.45 (s, 1H), 8.05 (d, 1H, J=5.5 Hz), 7.76 (s, 1H), 7.54-7.66 (m, 4H), 7.32 (t, 1H, J=8.1 Hz), 7.07-7.12 (m, 2H), 6.29 (d, 1H, J=5.5 Hz), 4.71 (s, 2H), 3.79-3.82 (m, 2H), 2.82-2.87 (m, 2H), 2.26-2.31 (m, 2H), 1.71-1.78 (m, 4H), 1.60-1.65 (m, 1H), 1.34-1.42 (m, 2H), and 1.14-1.28 (m, 2H); ESIMS: 622.40 (M+H⁺).

### Step E: N-(3-{2-Amino-5-[2-(1,2,3,4-tetrahydro-7-isoquinolinylamino)-4-pyrimidinyl]-1,3-thiazol-4-yl}phenyl)-N-methylcyclohexanecarboxamide

To a solution containing 250 mg (0.40 mmol) of N-{3-[2-amino-5-(2-{[2-(trifluoroacetyl)-1,2,3,4-tetrahydro-7-isoquinolinyl]amino}-4-pyrimidinyl)-1,3-thiazol-4-yl]phenyl}-N-methylcyclohexanecarboxamide and 5 mL of THF was added 30 mg of LiOH and 5 mL of water. The reaction mixture was heated at 50° C. for 1 h, cooled to rt, and extracted with DCM. The combined organic layers were dried over MgSO₄ and filtered, and the solvents were removed under reduced pressure. The residue was subjected to silica gel chromatography, eluting with a DCM/MeOH/NH₄OH mixture to give 180 mg (85%) of the title compound of Example 59 as a yellow solid: ¹H-NMR (d⁶-DMSO, 400 MHz) δ 9.86 (s, 1H), 9.30 (s, 1H), 8.02 (d, 1H, J=5.5 Hz), 7.74 (s, 1H), 7.64 (d, 1H, J=8.7 Hz), 7.59 (s, 2H), 7.49 (s, 1H), 7.36 (d, 1H, J=8.4 Hz), 7.31 (t, 1H, J=7.9 Hz), 7.10 (d, 1H, J=7.8 Hz), 6.92 (d, 1H, J=8.4 Hz), 6.24 (d, 1H, J=5.5 Hz), 3.84 (s, 2H), 2.91-2.94 (t, 2H, J=5.8 Hz), 2.58-2.62 (t, 2H, J=5.8 Hz), 2.25-2.31 (m, 2H), 1.69-1.77 (m, 4H), 1.58-1.62 (m, 1H), 1.35-1.42 (m, 2H), and 1.15-1.24 (m, 2H); HRMS calcd for C₂₉H₃₁N₇OS: 525.2311. Found: 526.2385 (M+H⁺).

### Example 60

### N-[3-(2-Amino-5-{2-[(4-{[3-(dimethylamino)propyl]amino}-3-fluorophenyl)amino]-4-pyrimidinyl}-1,3-thiazol-4-yl)phenyl]-N-methylcyclohexanecarboxamide

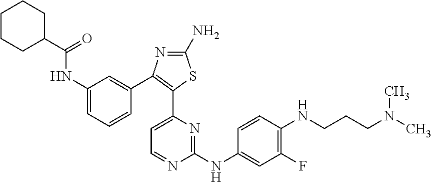

### Step A: N-{3-[2-Amino-5-(2-chloro-4-pyrimidinyl)-1,3-thiazol-4-yl]phenyl}-N-methylcyclohexanecarboxamide

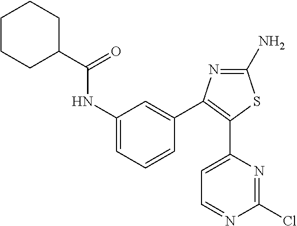

To a solution containing 1.5 g (4.19 mmol) of N-{3-[(2-chloro-4-pyrimidinypacetyl]phenyl}-N-methylcyclohexanecarboxamide, prepared by a procedure analogous to Example 59, Step B, and 10 mL of HOAc, was added 0.21 mL (4.19 mmol) of bromine. The reaction mixture was allowed to stir at rt for 1 h, then diluted in H₂O, neutralized by the careful addition of K₂CO₃, and extracted with DCM. The organic layers were dried over MgSO₄ and filtered, and the solvents were removed under reduced pressure. The residue was taken up in 20 mL of dioxane and 0.35 g (4.61 mmol) of thiourea and 0.39 g (4.61 mmol) of MgCO₃ were added. The reaction mixture was heated at 80° C. for 2 h, allowed to cool to rt, and diluted in H₂O. The mixture was filtered to give an insoluble and inseparable mixture of the title compound of Step A contaminated with a small amount of N-{3-[2-amino-5-(2-oxo-2,3-dihydro-4-pyrimidinyl)-1,3-thiazol-4-yl]phenyl}-N-methylcyclohexane-carboxamide, which was used without further purification.

### Step B: N-[3-(2-amino-5-{2-[(4-{[3-(dimethylamino)propyl]amino}-3-fluorophenyl)amino]-4-pyrimidinyl}-1,3-thiazol-4-yl)phenyl]-N-methylcyclohexanecarboxamide

To a solution containing 250 mg (0.60 mmol) of a mixture of N-{3-[2-amino-5-(2-chloro-4-pyrimidinyl)-1,3-thiazol-4-yl]phenyl}-N-methylcyclohexanecarboxamide and N-{3-[2-amino-5-(2-oxo-2,3-dihydro-4-pyrimidinyl)-1,3-thiazol-4-yl]phenyl}-N-methylcyclohexanecarboxamide, 1.5 mL of i-PrOH and 0.5 mL of DMA, was added 75 mg (0.3 mmol) N¹-[3-(dimethylamino)propyl]-2-fluoro-1,4-benzenediamine and one drop of conc HCl. The reaction was heated to 70° C. for 48 h, allowed to cool to rt, and neutralized by the addition of H₂O and aqueous 10% NaOH solution. The reaction mixture was filtered and the residue was subjected to silica gel chromatography, eluting with a DCM/MeOH/NH₄OH mixture, to give 53 mg (15%) of the title compound of Example 60 as an orange solid: ¹H-NMR (d⁶-DMSO, 400 MHz) δ 9.85 (s, 1H), 9.21 (s, 1H), 7.99 (d, 1H, J=5.3 Hz), 7.73 (s, 1H), 7.64 (d, 1H, J=8.2 Hz), 7.57 (s, 2H), 7.54 (d, 1H, J=2.2 Hz), 7.30 (t, 1H, J=7.9 Hz), 7.23 (d, 1H, J=9.7 Hz), 7.09 (d, 1H, J=7.6 Hz), 6.60 (t, 1H, J=9.3 Hz), 6.21 (d, 1H, J=5.3 Hz), 5.10 (s, 1H), 3.26 (s, 6H), 3.02-3.06 (m, 2H), 2.26-2.29 (m, 3H), 1.59-1.77 (m, 6H), 1.30-1.41 (m, 2H), and 1.15-1.25 (m, 4H); HRMS calcd for C₃₁H₃₇N₈OFS: 588.2795. Found: 589.2866 (M+H⁺).

### Example 61

### N-[3-(2-Amino-5-{2-[(3-chloro-4-{[2-(dimethylamino)ethyl]oxy}phenyl)amino]-4-pyrimidinyl}-1,3-thiazol-4-yl)phenyl]-N-methylcyclohexanecarboxamide

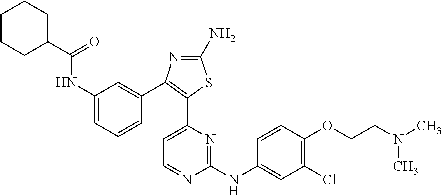

To a solution containing 250 mg (0.60 mmol) of a mixture of N-{3-[2-amino-5-(2-chloro-4-pyrimidinyl)-1,3-thiazol-4-yl]phenyl}-N-methylcyclohexanecarboxamide and N-{3-[2-amino-5-(2-oxo-2,3-dihydro-4-pyrimidinyl)-1,3-thiazol-4-yl]phenyl}-N-methylcyclohexanecarboxamide, prepared by a procedure analogous to Example 60, Step A, 1.5 mL of i-PrOH, and 0.5 mL of DMA, was added 130 mg (0.45 mmol) of 3-chloro-4-{[2-(dimethylamino)ethyl]oxy}phenylamine and one drop of conc HCl. The reaction was heated to 70° C. for 48 h, allowed to cool to rt, and neutralized by the addition of H₂O and an aqueous 10% NaHCO₃ solution. The reaction mixture was filtered and solvents were removed under reduced pressure. A DCM solution of the crude compound was treated with MP-isocyanate resin to remove excess phenylamine and then subjected to silica gel chromatography, eluting with a DCM/MeOH/NH₄OH mixture, to give 36 mg (10%) of the title compound of Example 61 as an orange solid: ¹H-NMR (d⁶-DMSO, 400 MHz) δ 9.92 (s, 1H), 9.51 (s, 1H), 8.04 (d, 1H, J=5.3 Hz), 7.91 (d, 1H, J=2.6 Hz), 7.70 (s, 1H), 7.53-7.64 (m, 4H), 7.30 (dd, 1H, J=7.9 and 7.9 Hz), 7.09 (d, 1H, J=7.8 Hz), 7.04 (d, 1H, J=9.0 Hz), 6.33 (d, 1H, J=5.3 Hz), 4.13 (t, 2H, J=5.8 Hz), 2.69-2.77 (m, 2H), 2.31 (s, 6H), 1.75-1.87 (m, 6H), 1.64-1.70 (m, 1H), 1.36-1.47 (m, 2H), and 1.18-1.32 (m, 2H); HRMS Calcd for C₃₀H₃₆ClN₇O₂S (M+H⁺): 592.2256. Found: 592.2256.

### Example 62

### N-{3-[2-Amino-5-(2-{[3-(1,3-oxazol-5-yl)phenyl]amino}-4-pyrimidinyl)-1,3-thiazol-4-yl]phenyl}cyclohexanecarboxamide

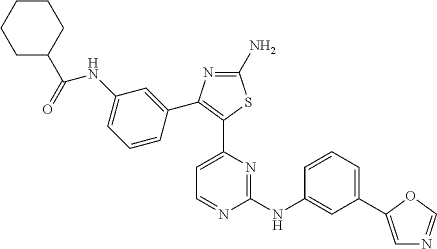

To obtain the title compound of Example 62, N-{3-[(E)-2-(2-chloro-4-pyrimidinyl-1-hydroxyethenyl]phenyl}cyclohexanecarboxamide (0.1 g, 0.28 mmol), prepared by a procedure analogous to Example 60, Step A, 3-(1,3-oxazol-5-yl)phenyl amine (0.065 g, 0.41 mmol), iPrOH (2 mL) and concentrated HCl (0.05 mL) were combined and heated at 80° C. overnight. NBS (0.03 g, 0.17 mmol) was added and the resulting mixture was allowed to stir at rt for 15 min. Thiourea (0.03 g, 0.47 mmol) and MgCO₃ N-hydrate (0.03 g) were added and the reaction was then heated 90° C. for 1 hr. The crude reaction was then diluted with EtOAc and H₂O. The desired compound was extracted into the organic layer which after concentrating yielded brown oil which was then purified using basic RP HPLC conditions. 50 mg (33%) of the title compound of Example 62 was obtained. ¹H-NMR (300 MHz, DMSO-d₆) δ 9.88 (s, 1H), 9.65 (s, 1 H), 8.46 (s, 1H), 8.21 (s, 1H), 8.11 (d, J=5.4 Hz, 1H), 7.78 (s, 1H), 7.75-7.62 (m, 4H), 7.61 (s, 1H), 7.40-7.29 (m, 3H), 7.14 (d, J=7.8 Hz, 1H), 6.34 (d, J=5.2 Hz, 1H), 2.30 (m, 1H), 1.84-1.58 (m, 5H), and 1.47-1.12 (m, 5H); MS (ESI) m/z 538 (M+H)⁺.

### Example 63

### N-{3-[5-{2-[(3-Chloro-4-{[2-(1-pyrrolidinyl)ethyl]oxy}phenyl)amino]-4-pyrimidinyl}-2-(ethylamino)-1,3-thiazol-4-yl]phenyl}cyclopropanecarboxamide

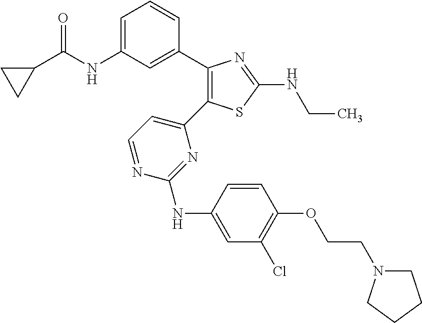

### Step A: Ethyl 3-[(cyclopropylcarbonyl)amino]benzoate

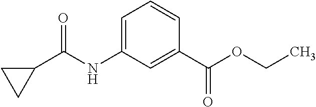

To obtain the target compound of Step A, ethyl 3-aminobenzoate (1.5 g, 9.0 mmol) was placed in a round bottom flask with DCM (10 mL). Cyclopropanecarbonyl chloride (1 mL) was added dropwise and the reaction was allowed to stir at rt until it was complete. DCM and H₂O were added to the reaction mixture and the desired product was extracted into the organic phase which was then concentrated to dryness to give 2.1 g of the desired product (quantitative yield). ¹H-NMR (400 MHz, DMSO-d₆) δ 10.42 (s, 1H), 8.23 (s, 1H), 7.84 (d, J=8.2 Hz, 1H), 7.59 (d, J=7.9 Hz, 1H), 7.41 (t, J=7.8 Hz, 1H), 4.29 (q, J=7.0 Hz, 2H), 1.77 (m, 1H), 1.29 (t, J=7.4 Hz, 3H), and 0.79 (m, 4H).

### Step B: N-{3-[(E)-2-(2-Chloro-4-pyrimidinyl)-1-hydroxyethenyl]phenyl}cyclopropanecarboxamide

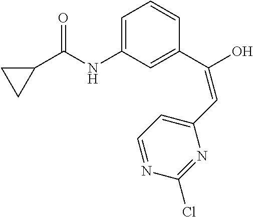

To obtain the desired compound, ethyl 3-[(cyclopropylcarbonyl)amino]-benzoate (2.1 g, 9.0 mmol) and LHMDS (30 mL, 29.7 mmol, 1 M in THF) were placed in a round bottom flask and cooled to 0° C. 2-Chloro-4-methylpyrimidine (1.2 g, 9.4 mmol) was added in one portion and the resulting mixture was allowed to stir and warm to rt overnight. EtOAc and H₂O were added to the reaction mixture and the desired product was extracted into the organic phase which was then concentrated onto silica gel and purified via column chromatography to yield 0.830 g of ketone/enolate mixture of the desired target compound (Yield: 29%). MS (ESI) m/z 316.10 (M+H)⁺.

### Step C: N-{3-[5-(2-Chloro-4-pyrimidinyl)-2-(ethylamino)-1,3-thiazol-4-yl]phenyl}cyclopropanecarboxamide

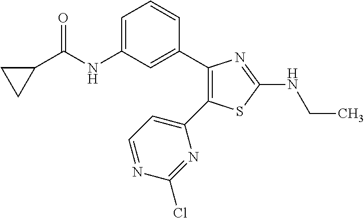

To obtain the desired compound, N-{3-[(E)-2-(2-chloro-4-pyrimidinyl)-1-hydroxyethenyl]phenyl}cyclopropanecarboxamide (0.83 g, 2.63 mmol) was dissolved in DMF (10 mL) in a round bottom flask with stirring. NBS (0.467 g, 2.63 mmol) was added in one portion and the resulting mixture was stirred at rt for 10 min. Ethylthiourea (0.301 g, 2.9 mmol) was then added and the reaction was stirred for about 1 hr at which time it was complete. EtOAc and H₂O were added to the reaction mixture and the desired product was extracted into the organic phase which was than concentrated onto silica gel and purified via column chromatography. Pure fractions were combined and concentrated to dryness. The resulting material was then sonicated in ether and the solid that persisted was filtered off to yield 475 mg of the desired compound (Yield: 45%). ¹H-NMR (400 MHz, DMSO-d₆) δ 10.31 (s, 1H), 8.54 (t, J=5.3 Hz, 1H), 8.26 (d, J=5.6 Hz, 1H), 7.73 (s, 1H), 7.70 (d, J=8.8 Hz, 1H), 7.38 (t, J=7.9 Hz, 1H), 7.14 (d, J=7.4 Hz, 1H), 6.78 (d, J=5.5 Hz, 1H), 3.30 (m, 2H), 1.75 (m, 1H), 1.18 (t, J=7.0 Hz, 3H), and 0.77 (m, 4H).

### Step D: N-{3-[5-{2-[(3-Chloro-4-{[2-(1-pyrrolidinyl)ethyl]oxy}phenyl)amino]-4-pyrimidinyl}-2-(ethylamino)-1,3-thiazol-4-yl]phenyl}cyclopropanecarboxamide

To obtain the title compound of Example 63, N-{3-[5-(2-chloro-4-pyrimidinyl)-2-(ethylamino)-1,3-thiazol-4-yl]phenyl}cyclopropanecarboxamide (0.098 g, 0.245 mmol) and 3-chloro-4-{[2-(1-pyrrolidinyl)ethyl]oxy}phenyl amine (0.068 g, 0.245 mmol), prepared by a procedure analogous to Example 27, Step B. were combined with i-PrOH (2 mL) and concentrated HCl (2 drops) in a microwave vial. The reaction was heated to 180° C. for 15 min in the microwave then cooled to rt. TEA (approx. 0.1 mL) and silica gel were combined with the reaction and the resulting mixture was concentrated to dryness. Column chromatography using EtOAc. MeOH and NH₄OH yielded fractions which were concentrated to dryness. The resulting material was then sonicated in ether and the solid that persisted was filtered off to yield 95 mg of the title compound of Example 63 (Yield: 64%). ¹H-NMR (400 MHz, DMSO-d₆) δ 10.25 (s, 1H), 9.45 (s, 1H), 8.23 (t, J=5.5 Hz, 1H), 8.05 (d, J=5.6 Hz, 1H), 8.00 (d, J=2.4 Hz, 1H), 7.70 (s, 1H), 7.67 (d, J=8.6 Hz, 1H), 7.49 (dd, J=9.1, and 2.5 Hz, 1H), 7.33 (t, J=8.0 Hz, 1H), 7.11 (d, J=8.0 Hz, 1H), 7.04 (d, J=8.8 Hz, 1H), 6.24 (d, J=5.5 Hz, 1H), 4.07 (t, J=5.9 Hz, 2H), 3.26 (m, 2H), 2.77 (t, J=6.0 Hz, 2H), 2.52 (m, 4H), 1.73 (m, 1H), 1.65 (m, 4H), 1.17 (m, 3H), and 0.76 (m, 4H); HRMS C₃₁H₃₆N₇O₂SCl (M+H)⁺ calcd 604.2261. found 604.2263.

### Example 64

### N-[3-(2-Amino-5-{2-[(3-fluorophenyl)amino]-4-pyrimidinyl}-1,3-thiazol-4-yl)phenyl]-2-(2-thienyl)acetamide

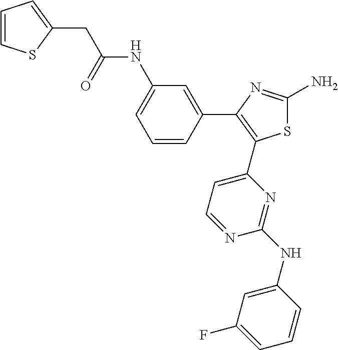

### Step A: 2-(2-Chloro-4-pyrimidinyl)-1-(3-nitrophenyl)ethanone

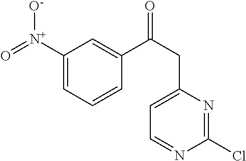

To a solution containing 12.2 g (95 mmol) of idine and 60 mL of THF at 0° C. was slowly added 190 mL (190 mmol) of a 1.6 M solution of LHMDS in THF. The reaction was allowed to stir for 6 h, then quenched by the addition of MeOH. The solvents were removed under reduced pressure and the residue was passed through a short plug of silica gel, eluting with EtOAc. The solvents were removed under reduced pressure and the resulting solid was further purified by trituration from an EtOAciether mixture to give 10.2 g (39%) of the title compound of Step A as a brown solid that exists as a mixture of ketone and enol tautomers: ESIMS: 278.25 (M+H⁺).

### Step B: 5-(2-Chloro-4-pyrimidinyl)-4-(3-nitrophenyl)-1,3-thiazol-2-amine

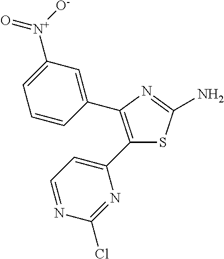

(Z)-2-(2-Chloro-4-pyrimidinyl)-1-(3-nitrophenyl)ethanol (3.86 g, 13.9 mmol) was dissolved in DCM (65 mL) and NBS (2.48 g, 13.90 mmol) was added. The reaction mixture was allowed to stir at rt for 15 min, then evaporated to dryness. The residue was dissolved in 100 mL of dioxane and MgCO₃ (3.9 g, 13.90 mmol) and thiourea (1.37 g, 18.07 mmol) were added. The reaction mixture was stirred at rt for 2 h, then diluted with EtOAc and quenched by the addition of 1.6 N HCl. The mixture was extracted with EtOAc, washed with brine, and dried over MgSO₄. The solvent was removed and the residue was absorbed onto silica and purified via flash chromatography, eluting with an EtOAc/Hexanes mixture. The desired fractions were combined to give 4.0 g (86%) of a yellow solid: ¹H-NMR (400 MHz, DMSO-D₆) δ 8.3 (m, 2H), 8.0 (d, J=7.9 Hz, 2H), 7.7 (t, J=8.0 Hz, 1H), 6.9 (d, J=5.7 Hz, 1H), and 2.5 (brs., 2H); ESIMS m/z 334 (M+H).

### Step C: 4-(3-Aminophenyl)-5-(2-chloro-4-pyrimidinyl)-1,3-thiazol-2-amine

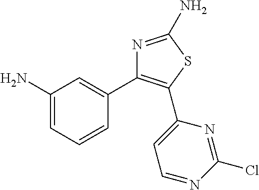

To a mixture containing 0.3 g (0.9 mmol) of 5-(2-chloro-4-pyrimidinyl)-4-(3-nitrophenyl)-1,3-thiazol-2-amine and 5 mL of EtOH was added 100 mg of 5% Platinum on carbon. The mixture was treated under a H₂ atmosphere at 60 psi for 12 h, and then 120 mg of 10% Pd on carbon was added. The reaction mixture was subjected to an atmosphere of H₂ at 60 psi for 4 days, and then filtered through a pad of Celite. The solvents were removed under reduced pressure to give 130 mg (48%) of 4-(3-aminophenyl)-5-(2-chloro-4-pyrimidinyl)-1,3-thiazol-2-amine: ¹H-NMR (400 MHz, DMSO-d₆) δ ppm 8.26 (d, J=5.49 Hz, 1H), 7.88 (s, 2H), 7.10 (t, J=7.41 Hz, 1H), 6.87 (d, J=5.67 Hz, 1H), 6.61-6.66 (m, 2H), 6.57 (d, J=7.68 Hz, 1H), and 5.24 (s, 2H); ESIMS: 304.04 (M+H⁺).

### Step D: N-{3-[2-Amino-5-(2-chloro-4-pyrimidinyl)-1,3-thiazol-4-yl]phenyl}-2-(2-thienyl)acetamide

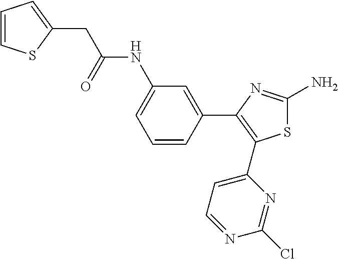

The title compound of Step D was prepared from 4-(3-aminophenyl)-5-(2-chloro-4-pyrimidinyl)-1,3-thiazol-2-amine (0.13 g, 0.43 mmol), and 2-thienylacetyl chloride (0.075 g, 0.47 mmol) by a procedure analogous to Example 1, Step A. Yield 51%.

### Step E: N-[3-(2-Amino-5-{2-[(3-fluorophenyl)amino]-4-pyrimidinyl}-1,3-thiazol-4-yl)phenyl]-2-(2-thienyl)acetamide

The title compound of Example 64 was prepared from N-{3-[2-amino-5-(2-chloro-4-pyrimidinyl)-1,3-thiazol-4-yl]phenyl}-2-(2-thienyl)acetamide (0.085 g, 0.20 mmol) and 3-fluorophenyl amine (0.022 g, 0.20 mmol) by a procedure analogous to Example 1, Step D. Yield 41 mg (41%). ¹H-NMR (300 MHz, DMSO-d₆) δ 10.28 (s, 1H), 9.72 (s, 1H), 8.11 (d, J=5.3 Hz, 1H), 7.85-7.75 (m, 2H), 7.69-7.62 (m, 3H), 7.44 (d, J=8.0 Hz, 1H), 7.40-7.33 (m, 2H), 7.26 (q, J=7.7 Hz, 1H), 7.17 (d, J=8.0 Hz, 1H), 6.97-6.94 (m, 2H), 6.72 (t, J=8.5 Hz, 1H), 6.34 (d, J=5.4 Hz, 1H), and 3.85 (s, 2H); HRMS C₂₆H₂₀N₆OFS₂ (M+H)⁺ calcd 503.1124. found 503.1125.

### Example 65

### N-[3-(2-Amino-5-{2-[(4-{[3-(dimethylamino)propyl]amino}-3-fluorophenyl)amino]-4-pyrimidinyl}-1,3-thiazol-4-yl)phenyl]-1-methyl-1H-pyrrole-2-carboxamide

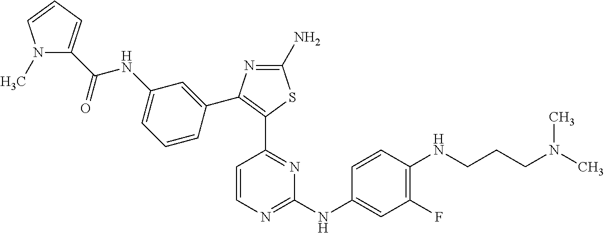

### Step A: Ethyl 3-{[(1-methyl-1H-pyrrol-2-yl)carbonyl]amino}benzoate

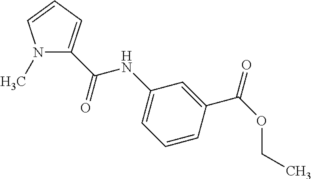

The title compound of Step A was prepared from ethyl 3-aminobenzoate (2.5 g, 15 mmol) and 1-methyl-1H-pyrrole-2-carbonyl chloride (2.4 g, 16.6 mmol) by a procedure analogous to Example 1, Step A. Yield 3.5 g (86%). ¹H-NMR (300 MHz, DMSO-d₆) δ 9.93 (s, 1H), 8.35 (s, 1H), 8.00 (d, J=8.4 Hz, 1H), 7.63 (d, J=7.7 Hz, 1H), 7.44 (t, J=7.9 Hz, 1H), 7.07 (dd, J=3.9, 1.7 Hz, 1H), 7.02 (s, 1H), 6.09 (m, 1H), 4.32 (q, J=7.1 Hz, 2H), 3.87 (s, 3H), and 1.32 (t, J=71 Hz, 3H).

### Step B: N-{3-[(2-Chloro-4-pyrimidinyl)acetyl]phenyl}-1-methyl-1H-pyrrole-2-carboxamide

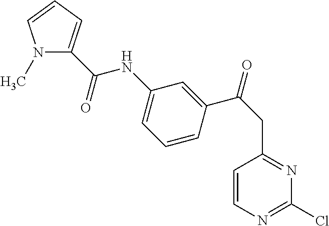

The title compound of Step B was prepared from ethyl 3-{[(1-methyl-1H-pyrrol-2-yl)carbonyl]amino}benzoate (3.5 g, 13 mmol), 2-chloro-4-methylpyrimidine (1.67 g, 13 mmol) and 1 M LiHMDS in THF (39 mL, 39 mmol) by a procedure analogous to Example 1, Step C. Yield 1.54 g (31%). MS (ESI) m/z 377 (M+H)⁺.

### Step C: N-{3-[2-Amino-5-(2-chloro-4-pyrimidinyl)-1,3-thiazol-4-yl]phenyl}-1-methyl-1H-pyrrole-2-carboxamide

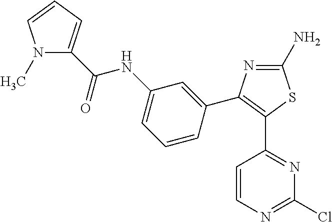

To obtain the title compound of Step C, N-{3-[(2-chloro-4-pyrimidinyl)acetyl]phenyl}-1-methyl-1H-pyrrole-2-carboxamide (0.4 g, 1.13 mmol) was treated with NBS (0.2 g, 1.13 mmol) followed by cyclization with thiourea (0.08 g, 1.24 mmol) in the presence of MgCO₃ N-hydrate (0.08 g) using a procedure analogous to Example 2, Step A. Yield 100 mg (22%). MS (ESI) m/z 411 (M+H)⁺.

### Step D: N-[3-(2-Amino-5-{2-[(4-{[3-(dimethylamino)propyl]amino}-3-fluorophenyl)amino]-4-pyrimidinyl}-1,3-thiazol-4-yl)phenyl]-1-methyl-1H-pyrrole-2-carboxamide

The title compound of Example 65 was prepared from N-{3-[2-amino-5-(2-chloro-4-pyrimidinyl)-1,3-thiazol-4-yl]phenyl}-1-methyl-1H-pyrrole-2-carboxamide (0.05 g, 0.12 mmol) and N-[3-(dimethylamino)propyl]-2-fluoro-1,4-benzenediamine hydrochloride (0.03 g, 0.12 mmol) by a method analogous to Example 1, Step D. Yield 15 mg (21%). ¹H-NMR (300 MHz, DMSO-d₆) δ 9.81 (s, 1H), 9.23 (s, 1H), 8.02 (d, J=5.5 Hz, 1H), 7.91 (s, 1H), 7.79 (d, J=8.5 Hz, 1H), 7.63-7.54 (m, 3H), 7.36 (t, J=8.1 Hz, 1H), 7.25 (d, J=9.2 Hz, 1H), 7.14 (d, J=7.9 Hz, 1H), 7.04-6.98 (m, 2H), 6.62 (t, J=9.3 Hz, 1H), 6.26 (d, J=5.3 Hz, 1H), 6.07 (m, 1H), 3.85 (s, 3H), 3.06 (t, J=7.2 Hz, 2H), 2.33 (t, J=7.0 Hz, 2H), 2.17 (s, 6H), and 1.69 (m, 2H); HRMS C₃₀H₃₃F₁N₆OS (M+H)⁺ calcd 586.2507. found 586.2507.

### Example 66

### N-[3-(2-Amino-5-{2-[(3-fluoro-4-{[2-(methyloxy)ethyl]oxy}phenyl)amino]-4-pyrimidinyl}-1,3-thiazol-4-yl)phenyl]-1-methyl-1H-pyrrole-2-carboxamide

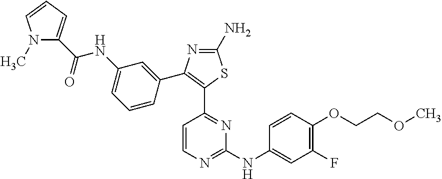

### Step A: N-[3-({2-[(3-Fluoro-4-{[2-(methyloxy)ethyl]oxy}phenyl)amino]-4-pyrimidinyl}acetyl)phenyl]-1-methyl-1H-pyrrole-2-carboxamide

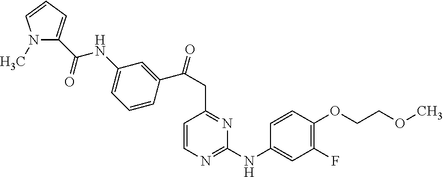

The title compound of Step A was prepared from N-{3-[(2-chloro-4-pyrimidinyl)acetyl]phenyl}-1-methyl-1H-pyrrole-2-carboxamide (0.2 g, 0.56 mmol), prepared by a procedure analogous to Example 65, Step B, and (3-fluoro-4-{[2-(methyloxy)ethyl]oxy}phenyl)amine hydrochloride (0.12 g, 0.56 mmol), by a procedure analogous to Example 1, Step D, and purified by filtration from i-PrOH. Yield 200 mg (70%). MS (ESI) m/z 504 (M+H⁺).

### Step B: N-[3-(2-Amino-5-{2-[(3-fluoro-4-{[2-(methyloxy)ethyl]oxy}phenyl)amino]-4-pyrimidinyl}-1,3-thiazol-4-yl)phenyl]-1-methyl-1H-pyrrole-2-carboxamide

The title compound of Example 66 was prepared from N-[3-({2-[(3-fluoro-4-{[2-(methyloxy)ethyl]oxy}phenypamino]-4-pyrimidinyl}acetyl)phenyl]-1-methyl-1H-pyrrole-2-carboxamide (0.1 g, 0.19 mmol), NBS (0.039 g, 0.218 mmol), thiourea (0.014 g, 0.218 mmol) and MgCO₃ N-hydrate (0.019 g) by a procedure analogous to Example 2, Step A, and purified by silica gel chromatography. Yield 30 mg (27%). ¹H-NMR (300 MHz, DMSO-d₆) δ 9.81 (s, 1H), 9.50 (s, 1H), 8.08 (d, J=5.3 Hz, 1H), 7.92 (s, 1H), 7.82-7.74 (m, 2H), 7.65 (s, 2H), 7.40-7.33 (m, 2H), 7.14 (d, J=7.6 Hz, 1H), 7.09-6.98 (m, 3H), 6.33 (d, J=5.4 Hz, 1H), 6.07 (m, 1H), 4.13-4.08 (m, 2H), 3.85 (s, 3H), 3.66-3.62 (m, 2H), and 3.30 (s, 3H); HRMS C₂₈H₂₇FN₇O₃S (M+H)⁺ calcd 560.1875. found 560.1871.

### Example 67

### N-[5-(2-Amino-5-{2-[3-chloro-4-{[2-(dimethylamino)ethyl]oxy}phenyl)amino]-4-pyrimidinyl}-1,3-thiazol-4-yl)-2-fluorophenyl]-2,6-difluorobenzamide

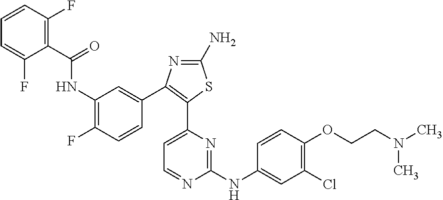

### Step A: Methyl 4-fluoro-3-nitrobenzoate

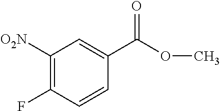

To a solution of 4-fluoro-3-nitrobenzoic acid (5.7 g, 30.8 mmol) in MeOH (50 mL), cooled to 0° C., was added thionyl chloride (3.37 mL, 46 mmol) dropwise over 10 min. The reaction was stirred overnight at rt. The MeOH was removed on the rotovap, and the residue was partitioned between EtOAc and saturated aqueous NaHCO₃. The aqueous layer was extracted with EtOAc, and the combined organic layers were dried over anhydrous Na₂SO₄, filtered, and concentrated to generate the desired product as a yellow solid (6.57 g, quantitative yield). ¹H-NMR-(400 MHz, d₆-DMSO): δ 8.56 (dd, 1H, J=2.2 Hz, 7.3 Hz), 8.29-8.33 (m, 1H), 7.73 (dd, 1H, J=8.9 Hz, 11 Hz), and 3.88 (s, 3H).

### Step B: Methyl 3-amino-4-fluorobenzoate

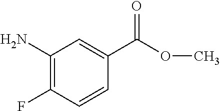

To a solution of methyl 4-fluoro-3-nitrobenzoate (6.57 g, 33 mmol) in EtOAc (75 mL) was added 10% palladium on carbon (0.66 g). The suspension was transferred to a Fisher-Porter flask and installed in a hydrogenation apparatus. The flask was charged with H₂ (25 psi) and stirred overnight at rt. The palladium was removed by filtration through a Celite plug, and the product was concentrated to generate a yellow oil, which darkened upon standing, in quantitative yield (5.51 g, 33 mmol). MS (ESI) m/z=170[M+H]⁺.

### Step C: Methyl 3-{[(2,6-difluorophenyl)carbonyl]amino}-4-fluorobenzoate

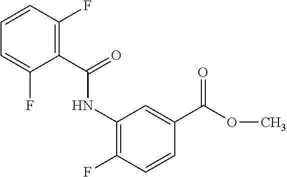

To a solution of methyl 3-amino-4-fluorobenzoate (5.5 g, 33 mmol) in DCM (100 mL) was added TEA (6.8 mL, 49 mmol) and 2,6-difluorobenzoyl chloride (4.3 mL, 34 mmol). After 90 min at rt, the reaction mixture was washed with H₂O and concentrated to a brown oil. The product was purified by column chromatography (eluting with 30-60% EtOAc/hexanes) to generate the desired product in 52% yield (5.37 g, 17 mmol). MS (ESI) m/z=310 [M+H]⁺.

### Step D: N-{5-[(2-Chloro-4-pyrimidinyl)acetyl]-2-fluorophenyl}-2,6-difluorobenzamide

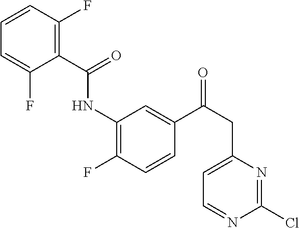

To a solution of methyl 3-{[(2,6-difluorophenyl)carbonyl]amino}-4-fluorobenzoate (3.0 g, 9.7 mmol) and 2-chloro-4-methylpyrimidine (1.2 g, 9.7 mmol) in anhydrous THF (10 mL) was added 1 M LHMDS in THF (30.1 mL, 30.1 mmol) dropwise over 30 min. The reaction was stirred overnight at rt. After 16 h, the reaction was poured over an ice/30 mL 1 M HCl mixture. The mixture was extracted 3× with EtOAc. The combined organic layers were dried over anhydrous Na₂SO₄, filtered, and adsorbed onto silica gel. The crude product was purified by column chromatography (eluting with 20-80% EtOAc/hexanes) to generate the desired product in 47% yield (1.86 g, 4.6 mmol). MS (ESI) m/z=406 [M+H]⁺.

### Step E: N-{5-[2-Amino-5-(2-chloro-4-pyrimidinyl)-1,3-thiazol-4-yl]-2-fluorophenyl}-2,6-difluorobenzamide

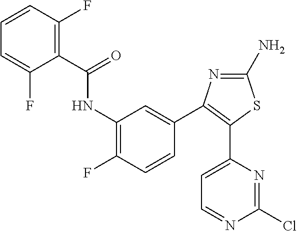

To a suspension of N-{5-[(2-chloro-4-pyrimidinypacetyl]-2-fluorophenyl}-2,6-difluorobenzamide (0.5 g, 1.2 mmol) in DCM (5 mL) was added NBS (220 mg, 1.2 mmol). After 30 min at rt, the DCM was removed on the rotovap, and the residue was redissolved in acetonitrile (2 mL). Thiourea (104 mg, 1.4 mmol) was added, and the reaction was stirred overnight at rt. The reaction mixture was adsorbed onto silica gel and purified by column chromatography (eluting with 10-100% EtOAc/DCM) to generate the desired product of Step E in 57% yield (314 mg, 0.68 mmol). MS (ESI) m/z=462 [M+H]⁺.

### Step F: N-[5-(2-Amino-5-{2-[(3-chloro-4-{[2-(dimethylamino)ethyl]oxy}phenyl)amino]-4-pyrimidinyl}-1,3-thiazol-4-yl)-2-fluorophenyl]-2,6-difluorobenzamide

To a suspension of N-{5-[2-amino-5-(2-chloro-4-pyrimidinyl)-1,3-thiazol-4-yl]-2-fluorophenyl}-2,6-difluorobenzamide (90 mg, 0.19 mmol) and {2-[(4-amino-2-chlorophenyl)oxy]ethyl}dimethylammonium chloride (49 mg, 0.19 mmol) in iPrOH (3 mL) was added 1 drop of concentrated HCl. The reaction was stirred overnight at 90° C. After 20 h, the reaction was quenched by the addition of 2 drops of TEA, and the solvent was removed on the rotovap. The residue was redissolved in 2 mL 4:1 MeOH/DMSO and purified by reverse-phase HPLC (eluting with 20-60% acetonitrile/0.1% trifluoroacetic acid). The product fractions were combined, basified to pH>9 with Na₂CO₃, and extracted with EtOAc. The combined organic layers were dried over anhydrous Na₂SO₄, filtered, and concentrated to generate the desired product of Example 67 in 23% yield (28 mg, 0.04 mmol). ¹H-NMR (400 MHz, d₆-DMSO): δ 10.78 (s, 1H), 9.49 (s, 1H), 8.12 (d, 1H, J=5.3 Hz), 8.07 (d, 1H, J=7.9 Hz), 7.93 (d, 1H, J=2.6 Hz), 7.68 (s, 2H), 7.55-7.58 (m, 2H), 7.34-7.40 (m, 2H), 7.23 (t, 2H, J=7.9 Hz), 7.06 (d, 1H, J=9.0 Hz), 6.40 (d, 1H, J=5.7 Hz), 4.07 (t, 2 H, J=5.8 Hz), 2.63 (t, 2H, J=5.7 Hz), and 2.23 (s, 6H). MS (ESI) m/z=640 [M+H]⁺.

### Example 68

### N-[3-(2-Amino-5-{2-[(3-chloro-4-{[2-(dimethylamino)ethyl]oxy}phenyl)amino]-4-pyrimidinyl}-1,3-thiazol-4-yl)-4-fluorophenyl]-2,6-difluorobenzamide

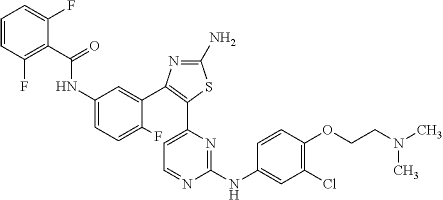

### Step A: Methyl 5-{[(2,6-difluorophenyl)carbonyl]amino}-2-fluorobenzoate

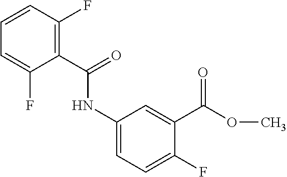

To a solution of methyl 5-amino-2-fluorobenzoate (2.5 g, 14.8 mmol) (prepared according to literature precedence) in DCM (100 mL) at 0° C., 2,6-difluorobenzoyl chloride (1.85 mL, 14.8 mmol) was added and the solution was allowed to stir for 30 min. The reaction mixture was quenched with H₂O (50 mL). The organic layer was then washed with 1M NaOH (50 mL), dried over MgSO₄, filtered and evaporated onto silica gel. The title compound of Step A was purified by chromatography, (25-100% EtOAc in hexanes). The title compound of Step A was obtained as a white solid in 69% yield (3.17 g). MS (ESI): 309.99 [M+H]⁺.

### Step B: N-{3-[(2-Chloro-4-pyrimidinyl)acetyl]-4-fluorophenyl}-2,6-difluorobenzamide

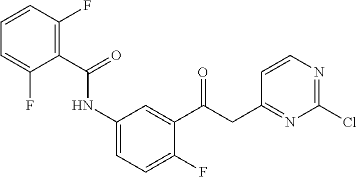

The title compound of Step B was prepared from methyl 5-{[(2,6-difluorophenyl)carbonyl]amino}-2-fluorobenzoate (3.17 g, 10.3 mmol) and 2-chloro-4-methylpyrimidine (1.33 g, 10.3 mmol) by a procedure analogous to Example 1, Step C. The title compound of Step B was isolated in 75% yield as a peach solid (3.1 g). MS (ESI): 406.12 [M+H]⁺.

### Step C: N-{3-[2-Amino-5-(2-chloro-4-pyrimidinyl)-1,3-thiazol-4-yl]-4-fluorophenyl}-2,6-difluorobenzamide

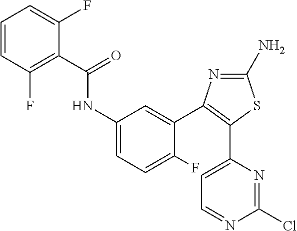

To a suspension of N-{3-[(2-chloro-4-pyrimidinyl)acetyl]-4-fluorophenyl}-2,6-difluorobenzamide (0.5 g, 1.2 mmol) in DCM (5 mL) was added NBS (220 mg, 1.2 mmol). After 30 min at rt, the DCM was removed on the rotovap, and the residue was redissolved in dioxane (2 mL). Thiourea (103 mg, 1.4 mmol) was added, and the reaction was stirred overnight at rt. The reaction mixture was concentrated and partitioned between EtOAc and 1 N aqueous NaOH. The organic layer was adsorbed onto silica gel and purified by column chromatography (eluting with 20-100% EtOAc/DCM) to generate the desired product of Step C in 67% yield (0.38 g, 0.8 mmol). MS (ESI) m/z=462 [M+H]⁺.

### Step D: N-[3-(2-Amino-5-{2-[(3-chloro-4-{[2-(dimethylamino)ethyl]oxy}phenyl)amino]-4-pyrimidinyl}-1,3-thiazol-4-yl)-4-fluorophenyl]-2,6-difluorobenzamide

To a suspension of N-{3-[2-amino-5-(2-chloro-4-pyrimidinyl)-1,3-thiazol-4-yl]-4-fluorophenyl}-2,6-difluorobenzamide (100 mg, 0.22 mmol) and {2-[(4-amino-2-chlorophenyl)oxy]ethyl}dimethylammonium chloride (54 mg, 0.22 mmol) in iPrOH (2 mL) was added 2 drops of concentrated HCl. The reaction was heated to 180° C. for 10 min in a microwave reactor. The reaction mixture was concentrated on the rotovap, and the residue was redissolved in 2 mL 1:1 MeOH/DMSO and purified by reverse-phase HPLC (eluting with 10-50% acetonitrile/0.1% TFA). The product fractions were combined, basified to pH>9 with Na₂CO₃, and extracted with EtOAc. The combined organic layers were dried over anhydrous Na₂SO₄, filtered, concentrated, and lyophilized from MeOH/H₂O to generate the desired product of Example 68 in 66% yield (93 mg, 0.15 mmol). ¹H-NMR (400 MHz, d₆-DMSO): δ 10.98 (s, 1H), 9.49 (s, 1H), 8.15 (d, 1H, J=5.5 Hz), 7.90-7.95 (m, 2H), 7.53-7.63 (m, 3H), 7.23-7.34 (m, 4H), 7.05 (d, 1H, J=9.1 Hz), 6.22 (d, 1H, J=5.1 Hz), 4.07 (t, 2H, J=5.9 Hz), 2.63 (t, 2H, J=5.8 Hz), and 2.23 (s, 6H). MS (ESI) m/z=640 [M+H]⁺.

### Example 69

### 4-(2-Amino-4-{3-[(phenylmethyl)oxy]phenyl}-1,3-thiazol-5-yl)-N-[3-(1,3-oxazol-5-yl)phenyl]-2-pyrimidinamine

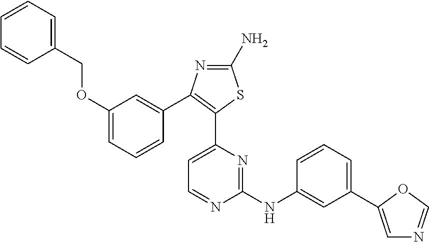

### Step A: Ethyl 3-[(phenylmethyl)oxy]benzoate

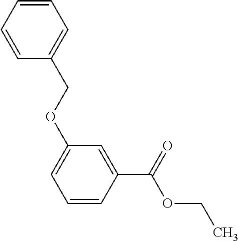

To a solution of 2.0 g (12.0 mmol) ethyl 3-hydroxybenzoate and 5 mL of DMF was added 1.8 g (13.2 mmol) of K₂CO₃ and 1.5 mL (12.6 mmol) of benzyl bromide. The reaction mixture was allowed to stir at rt for 13 h, then diluted in ether and washed with H₂O and brine. The organic layer was dried over MgSO₄ and filtered, and the solvent was removed under reduced pressure to give a quantitative yield of the title compound of Step A as clear oil: ¹H-NMR (CDCl₃, 400 MHz) δ 7.63-7.69 (m, 2H), 7.31-7.44 (m, 6H), 7.14 (m, 1H), 5.10 (s, 2H), 4.36 (q, 2H, J=7.1 Hz), and 1.38 (t, 3H, J=7.1 Hz); ESIMS: 255.10 M−H⁻).

### Step B: 2-(2-Chloro-4-pyrimidinyl)-1-{3-[(phenylmethyl)oxy]phenyl}ethanone

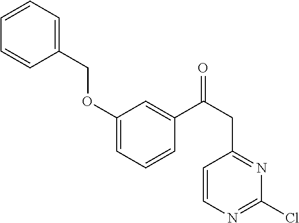

To a solution containing 2.0 g (7.8 mmol) of ethyl 3-[(phenylmethyl)oxy]benzoate and 4.9 mL (7.8 mmol) of a 1.6 M solution of 2-chloro-4-methylpyrimidine in THF at 0° C. was slowly added 15.6 mL (15.6 mmol) of a 1.0 M solution of LHMDS in THF. The reaction mixture was allowed to warm to rt and stir for 13 h, quenched by the addition of 10% aqueous HCl and extracted with DCM. The combined organic layers were dried over MgSO₄ and filtered, and the solvents were removed under reduced pressure. The residue was subjected to silica gel chromatography, eluting with an EtOAc/hexane mixture, to give 1.4 g (53%) of the title compound of Step B as a yellow oil that exists as a mixture of ketone and enol tautomers: ESIMS: 339.10 (M+H⁺).

### Step C: 5-(2-Chloro-4-pyrimidinyl)-4-{3-[(phenylmethyl)oxy]phenyl}-1,3-thiazol-2-amine

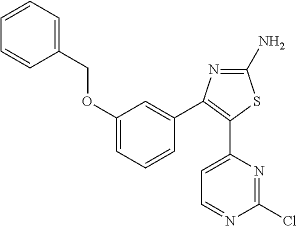

To a solution containing 340 mg (10 mmol) of 2-(2-chloro-4-pyrimidinyl)-1-{3-[(phenylmethypoxy]phenyl}ethanone and 5 mL of HOAc was added 51 μL (1.0 mmol) of bromine. The reaction mixture was allowed to stir at rt for 1 h, then diluted with water, quenched by the addition of 10% aqueous NaOH, and extracted with DCM. The combined organic layers were dried over MgSO₄ and filtered, and the solvents were removed under reduced pressure. The residue was dissolved in 10 mL of dioxane and 84 mg (1.1 mmol) of thiourea, and 93 mg (1.1 mmol) of MgCO₃ was added. The reaction mixture was heated to 50° C. for 12 h, and then diluted in H₂O and extracted with DCM. The combined organic layers were dried over MgSO₄ and filtered, and the solvents were removed under reduced pressure. The residue was subjected to silica gel chromatography to give 280 mg (72%) of the title compound of Step C as an orange solid: ¹H-NMR (d⁶-DMSO, 400 MHz) δ 8.21 (d, 1H, J=5.5 Hz), 7.92 (s, 2H), 7.29-7.42 (m, 6H), 7.09-7.11 (m, 2H), 7.05 (d, 1H, J=7.5 Hz), 6.76 (d, 1H, J=5.5 Hz), and 5.10 (s, 2H); ESIMS: 395.10 (M+H⁺).

### Step D: 4-(2-Amino-4-{3-[(phenylmethyl)oxy]phenyl}-1,3-thiazol-5-yl)-N-[3-(1,3-oxazol-5-yl)phenyl]-2-pyrimidinamine

To a solution containing 95 mg (0.17 mmol) of 5-(2-chloro-4-pyrimidinyl)-4-{3-[(phenylmethyl)oxy]phenyl}-1,3-thiazol-2-amine, 1 mL of i-PrOH, and 0.5 mL of DMA, was added 27 mg (0.17 mmol) of 3-(1,3-oxazol-5-yl)phenyl amine and 1 drop of conc HCl. The reaction mixture was heated at 70° C. for 48 h, neutralized by the addition of aqueous NaHCO₃, and extracted with DCM. The combined organic layers were dried over MgSO₄ and filtered, and the solvent was removed under reduced pressure. The residue was subjected to silica gel chromatography, eluting with an EtOAc/hexane mixture, and the DMA was removed by triturating from DCM to give 66 mg (76%) of the title compound of Example 69 as a yellow solid: ¹H-NMR (d⁶-DMSO, 400 MHz) δ 9.65 (s, 1H), 8.46 (s, 1H), 8.20 (s, 1H), 8.08 (d, 1H, J=5.5 Hz), 7.72 (d, 1H, J=7.8 Hz), 7.61 (s, 2H), 7.30-7.43 (m, 8H), 7.12 (s, 1H), 7.06-7.09 (m, 2H), 6.29 (d, 1H, J=5.5 Hz), and 5.10 (s, 2H); HRMS Calcd for C₂₉H₂₂N₆O₂S: 518.1525. Found: 519.1603 (M+H⁺).

### Example 70

### 4-(2-Amino-4-{3-[(phenylmethyl)oxy]phenyl}-1,3-thiazol-5-yl)-N-(3-chloro-4-{[2-(dimethylamino)ethyl]oxy}phenyl)-2-pyrimidinamine

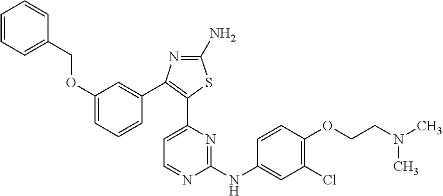

To a solution containing 95 mg (0.17 mmol) of 5-(2-chloro-4-pyrimidinyl)-4-{3-[(phenylmethyl)oxy]phenyl}-1,3-thiazol-2-amine, prepared by a procedure analogous to Example 67, Step C, 1 mL of i-PrOH and 0.5 mL of DMA, was added 27 mg (0.17 mmol) of 3-chloro-4-{[2-(dimethylamino)ethyl]oxy}phenyl amine and 1 drop of conc HCl. The reaction mixture was heated at 70° C. for 120 h, neutralized by the addition of aqueous NaNCO₃, and extracted with DCM. The combined organic layers were dried over MgSO₄ and filtered, and the solvent was removed under reduced pressure. The residue was subjected to silica gel chromatography, eluting with a DCM/MeOH/NH₄OH, and the co-eluting phenyl amine was removed by treatment of a DCM solution of the compound with MP-isocyante to give 12 mg (13%) of the title compound of Example 70 as an orange solid: ¹H-NMR (d⁶-DMSO, 400 MHz) δ 9.51 (s, 1H), 8.09 (d, 1H, J=5.4 Hz), 7.99 (d, 1H, J=2.6 Hz), 7.67 (s, 2H), 7.61 (dd, 1 H, J=9.0 and 2.7 Hz), 7.35-7.48 (m, 5H), 7.09-7.15 (m, 3H), 6.29 (d, 2H, J=9.4 Hz), 5.14 (s, 2H), 4.13 (t, 1H, J=5.8 Hz), 2.65 (brs, 2H), and 2.30 (s, 6H); HRMS Calcd for C₃₀H₂₉ClN₆O₂S: 572.1761. Found: 573.1834 (M+H⁺).

### Example 71

### N-[4-(2-Amino-4-{3-[(phenylmethyl)oxy]phenyl}-1,3-thiazol-5-yl)-2-pyrimidinyl]-1,2,3,4-tetrahydro-7-isoquinolinamine

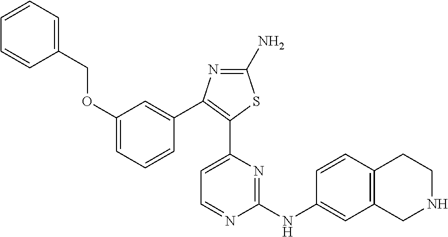

### Step A: N-[4-(2-Amino-4-{3-[(phenylmethyl)oxy]phenyl}-1,3-thiazol-5-yl)-2-pyrimidinyl]-2-(trifluoroacetyl)-1,2,3,4-tetrahydro-7-isoquinolinamine

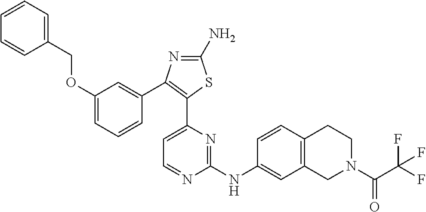

To a solution containing 95 mg (0.17 mmol) of 5-(2-chloro-4-pyrimidinyl)-4-{3-[phenylmethyl)oxy]phenyl}-1,3-thiazol-2-amine, prepared by a procedure analogous to Example 69, Step C, 1 mL of i-PrOH and 0.5 mL of DMA, was added 27 mg (0.17 mmol) of 2-(trifluoroacetyl)-1,2,3,4-tetrahydro-7-isoquinolinamine and 1 drop of conc HCl. The reaction mixture was heated at 70° C. for 48 h, neutralized by the addition of aqueous NaHCO₃, and extracted with DCM. The combined organic layers were dried over MgSO₄ and filtered, and the solvent was removed under reduced pressure. The residue was subjected to silica gel chromatography, eluting with a DCM/MeOH mixture to give 77 mg (76%) of the title compound of Step A as an orange solid: ¹H-NMR (d⁶-DMSO, 400 MHz) δ 9.44 (s, 1H), 8.03 (d, 1H, J=5.5 Hz), 7.52-7.59 (m, 4H), 7.27-7.41 (m, 6H), 7.03-7.09 (m, 4H), 6.23 (d, 1H, J=5.5 Hz), 5.08 (s, 2H), 4.69 (s, 2H), 3.77-3.80 (m, 2H), and 2.80-2.85 (m, 2H); ESIMS: 603.30 (M+H⁺).

### Step B: N-[4-(2-Amino-4-{3-[phenylmethyl)oxy]phenyl}-1,3-thiazol-5-yl)-2-pyrimidinyl]-1,2,3,4-tetrahydro-7-isoquinolinamine

To a solution containing 77 mg (0.13 mmol) of N-[4-(2-amino-4-{3-[(phenylmethyl)oxy]phenyl}-1,3-thiazol-5-yl)-2-pyrimidinyl]-2-(trifluoroacetyl)-1,2,3,4-tetrahydro-7-isoquinolinamine and 1 mL of THF was added 5 mg (0.26 mmol) of LiOH and 1 mL of H₂O. The reaction mixture was allowed to stir at rt for 2 h, then heated at 50° C. for an additional 1 h. The reaction mixture was allowed to cool to rt, then diluted in H₂O and extracted with DCM. The combined organic layers were collected and washed with aqueous NaHCO₃, dried over MgSO₄ and filtered, and the solvent was removed under reduced pressure to give 21 mg (32%) of the title compound of Example 71 as a yellow solid: ¹H-NMR (d⁶-DMSO, 400 MHz) δ 9.32 (s, 1H), 8.01 (d, 1H, J=5.5 Hz), 7.60 (s, 2H), 7.50 (s, 1H), 7.27-7.41 (m, 7H), 7.02-7.08 (m, 3H), 6.93 (d, 1H, J=8.5 Hz), 6.20 (d, 1H, J=5.5 Hz), 5.08 (s, 2H), 3.86 (s, 2H), 2.93-2.97 (m, 2H), and 2.60-2.64 (m, 2H); HRMS Calcd for C₂₉H₂₆N₆OS: 506.1889. Found: 507.1967 (M+H⁺).

### Example 72

### N-{3-[5-{2-[(3-Chloro-4-{[2-(dimethylamino)ethyl]oxy}phenyl)amino]-4-pyrimidinyl}-2-(1-piperidinyl)-1,3-thiazol-4-yl]phenyl}-2,6-difluorobenzamide

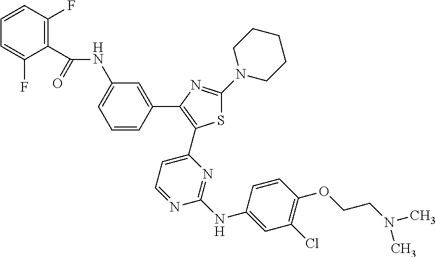

StepA: N-{3-[5-(2-Chloro-4-pyrimidinyl)-2-(1-piperidinyl)-1,3-thiazol-4-yl]phenyl}-2,6-difluorobenzamide

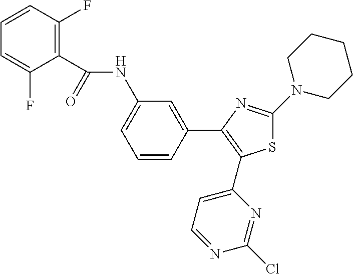

The title compound of Step A was prepared from N-{3-[(2-chloro-4-pyrimidinyl)acetyl]phenyl}-2,6-difluorobenzamide (0.4 g, 1.03 mmol), prepared in a procedure analogous to Example 1, Step C, NBS (0.184 g, 1.03 mmol) and 1-piperidinecarbothioamide (0.15 g, 1.03 mmol), by a procedure analogous to Example 2, Step A. yield 480 mg (91%). ¹H-NMR (400 MHz, DMSO-d₆) δ 10.94 (s, 1H), 8.29 (d, J=5.9 Hz, 1H), 7.84 (s, 1H), 7.80 (d, J=8.8 Hz, 1H), 7.58 (m, 1H), 7.46 (t, J=7.9 Hz, 1H), 7.29-7.21 (m, 3H), 6.86 (d, J=5.4 Hz, 1H), 3.56 (bsr, 4H), and 1.61 (bsr, 6H); MS (ESI) m/z 512 (M+H)⁺.

### Step B: 2,6-Difluoro-N-{3-[5-(2-methyl-4-pyrimidinyl)-2-(1-piperidinyl)-1,3-thiazol-4-yl]phenyl}benzamide

The title compound of Example 72 was prepared from N-{3-[5-(2-chloro-4-pyrimidinyl)-2-(1-piperidinyl)-1,3-thiazol-4-yl]phenyl}-2,6-difluorobenzamide (0.11 g, 0.21 mmol) and {2-[(4-amino-2-chlorophenyl)oxy]ethyl}dimethylamine dihydrochloride (0.054 g, 0.21 mmol) by a procedure analogous to Example 1, Step D. Yield 61 mg (41%). ¹H-NMR (400 MHz, DMSO-d₆) δ 10.92 (s, 1H), 9.52 (s, 1H), 8.13-8.05 (m, 2 H), 7.83 (s, 1H), 7.78 (d, J=77 Hz, 1H), 7.57 (m, 1H), 7.50-7.40 (m, 2H), 7.30-7.18 (m, 3H), 7.08 (d, J=9.2 Hz, 1H), 6.31 (d, J=5.5 Hz, 1H), 4.06 (t, J=5.8 Hz, 2H), 3.53 (s, 4H), 2.62 (t, J=5.7 Hz, 2H), 2.22 (s, 6H), and 1.61 (s, 6H); HRMS C₃₅H₃₅N₇O₂F₂SCl (M+H)⁺ calcd 690.2230. found 690.2242.

### Example 73

### 2,6-Difluoro-N-{3-[2-(1-piperidinyl)-5-(2-{[3-(1-pyrrolidinylmethyl)phenyl]amino}-4-pyrimidinyl)-1,3-thiazol-4-yl]phenyl}benzamide

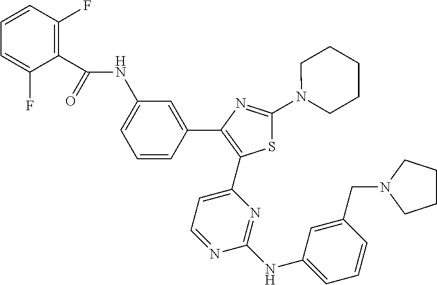

The title compound of Example 73 was prepared from N-{3-[5-(2-chloro-4-pyrimidinyl)-2-(1-piperidinyl)-1,3-thiazol-4-yl]phenyl}-2,6-difluorobenzamide (0.11 g, 0.21 mmol), prepared by a procedure analogous to Example 70, Step A, and 3-(1-pyrrolidinylmethyl)phenyl amine (0.038 g, 0.21 mmol), by a procedure analogous to Example 1, Step D. Yield 57 mg (41%). ¹H-NMR (400 MHz, DMSO-d₆) δ 10.94 (s, 1H), 9.53 (s, 1H), 8.13 (d, J=5.2 Hz, 1H), 7.93 (s, 1H), 7.86 (s, 1H), 7.80 (d, J=7.9 Hz, 1H), 7.60 (m, 1H), 7.53-7.43 (m, 2H), 7.30-7.18 (m, 4H), 6.93 (d, J=7.0 Hz, 1H), 6.34 (d, J=5.4 Hz, 1H), 3.64 (brs, 2H), 3.55 (s, 4H), 2.49 (s, 4H), 1.71 (s, 4H), and 1.63 (s, 6H); HRMS C₃₆H₃₆N₇OF₂S (M+H)⁺ calcd 652.2670. found 652.2668.

### Example 74

### N-{3-[5-{2-[(3-{[2-(Dimethylamino)ethyl]oxy}phenyl)amino]-4-pyrimidinyl}-2-(1-piperidinyl)-1,3-thiazol-4-yl]phenyl}-2,6-difluorobenzamide

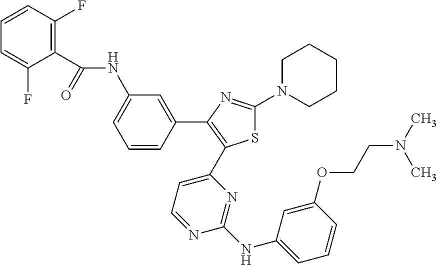

The title compound of Example 74 was prepared from N-{3-[5-(2-chloro-4-pyrimidinyl)-2-(1-piperidinyl)-1,3-thiazol-4-yl]phenyl}-2,6-difluorobenzamide (0.11 g, 0.21 mmol), prepared by a procedure analogous to Example 72, Step A, and {2-[(3-aminophenyl)oxy]ethyl}dimethylamine hydrochloride (0.046 g, 0.21 mmol), by a procedure analogous to Example 1, Step D. Yield 93 mg (66%). ¹H-NMR (400 MHz, DMSO-d₆) δ 10.92 (s, 1H), 9.50 (s, 1H), 8.12 (d, J=5.5 Hz, 1H), 7.84 (s, 1H), 7.80 (d, J=8.6 Hz, 1H), 7.63 (s, 1H), 7.59 (t, J=7.7 Hz, 1H), 7.45 (t, J=7.9 Hz, 1H), 7.30-7.21 (m, 3H), 7.20-7.11 (m, 2H), 6.51 (d, J=7.8 Hz, 1H), 6.33 (d, J=5.3 Hz, 1H), 4.06 (m, 2H), 3.54 (s, 4H), 2.62 (m, 2H), 2.21 (s, 6H), and 1.63 (s, 6H); HRMS C₃₅H₃₆N₇O₂F₂S (M+H)⁺ calcd 656.2619. found 656.2626.

### Example 75

### 3-(2-Amino-5-{2-[(3-chloro-4-{[2-(dimethylamino)ethyl]oxy}phenyl)amino]-4-pyrimidinyl}-1,3-thiazol-4-yl)-N-(2,6-difluorophenyl)benzamide

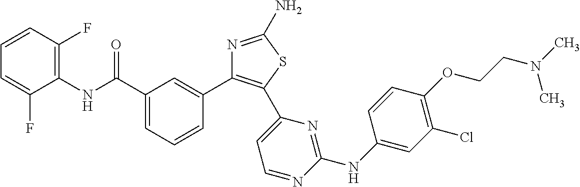

### Step A: Methyl 3-{[(2,6-difluorophenyl)amino]carbonyl}benzoate

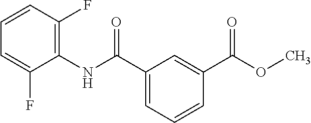

To a solution of 3-[(methyloxy)carbonyl]benzoic acid (4.0 g, 22 mmol) in DCM (200 ml) was added oxalyl chloride (8.46 g, 66.6 mmol) followed by DMF (1.0 mL). After stirring at rt for 48 h, solvent was removed, residue resuspended in DCM (200 mL), and the resulting mixture was concentrated again under reduced pressure. After placing the crude acid chloride on the high vacuum for 2 h, the solids were redissolved in DCM (250 mL) and to this was added pyridine (8.79 g, 111 mmol), catalytic DMAP, and 2,6-difluorophenyl amine (8.60 g, 67 mmol). After stirring at rt overnight, the reaction was washed with 1 M HCl (100 mL), organic layer separated, and purified by silica gel chromatography, eluting with a mixture of DCM and MeOH to provide the product (5.57 g, 86%) as an off-white solid. ESIMS (M+H)+=292.

### Step B: 3-[(2-Chloro-4-pyrimidinyl)acetyl]-N-(2,6-difluorophenyl)benzamide

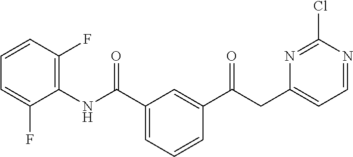

To an oven-dried flask under N₂ was added methyl 3-{[(2,6-difluorophenyl)amino]carbonyl}benzoate (860 mg, 2.95 mmol), 2-chloro-4-methylpyrimidine (760 mg, 5.94 mmol), and anhydrous THF (150 mL). The reaction mixture was then cooled to 0° C. and to this was added LHMDS (1.0 M in THF, 10.53 g, 11.82 mmol) via a slow dropwise addition. The resulting solution was allowed to warm to rt, stirred for 1 hr, and poured into ice water. The pH of the aqueous layer was adjusted to 4 with 1 M HCl, extracted with EtOAc (200 mL), organic layer separated, concentrated under reduced pressure, and purified by LC (DCM to 2% MeOH/DCM) to provide the product (0.85 g, 75%) as a tan solid. ESIMS (M+H)+=388.

### Step C: 3-[2-Amino-5-(2-chloro-4-pyrimidinyl)-1,3-thiazol-4-yl]-N-(2,6-difluorophenyl)benzamide

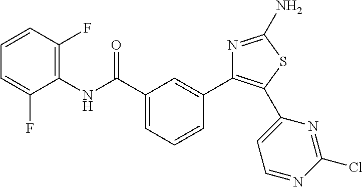

To a suspension of 3-[(2-chloro-4-pyrimidinyl)acetyl]-N-(2,6-difluorophenyl)benzamide (0.50 g, 1.3 mmol) in DCM (5 mL) was added NBS (0.23 g, 1.3 mmol). After 30 min at rt, the suspended solids had completely dissolved. The DCM was removed in vacuo, and the residue was redissolved in acetonitrile (2 mL). Thiourea (109 mg, 1.4 mmol) was added in one portion, and the reaction was stirred for 2 hours at rt. The reaction mixture was adsorbed onto silica and purified by column chromatography to give the title compound of Step C. (0.44 g, 77% yield). ¹H-NMR (400 MHz, d₆-DMSO): δ 11.05 (s, 2H), 10.19 (s, 1H), 8.31 (d, J=4.2 Hz, 1H), 8.20 (s, 1H), 8.10 (d, J=7.7 Hz, 1H), 7.78 (d, J=7.2 Hz, 1H), 7.63 (t, J=7.4 Hz, 1H), 7.39-7.42 (m, 1H), 7.21 (t, J=7.9 Hz, 2H), and 6.86 (d, J=4.6 Hz, 1H); MS (ESI) m/z=444 [M+H]⁺.

### Step D: 3-(2-Amino-5-{2-[(3-chloro-4-{[2-(dimethylamino)ethyl]oxy}phenyl)amino]-4-pyrimidinyl}-1,3-thiazol-4-yl)-N-(2,6-difluorophenyl)benzamide

To a suspension of 3-[2-amino-5-(2-chloro-4-pyrimidinyl)-1,3-thiazol-4-yl]-N-(2,6-difluorophenyl)benzamide (90 mg, 0.20 mmol) and {2-[(4-amino-2-chlorophenyl)oxy]ethyl}dimethylammonium chloride (51 mg, 0.20 mmol) in i-PrOH (3.0 mL), in a 10-mL microwave vial, was added 4 N HCl in dioxane (50 μL, 0.2 mmol). The vial was capped and heated in the microwave for 30 min at 170° C. The crude product was purified by flash chromatography and reversed-phase HPLC to generate the title compound of Example 75 in 54% yield. ¹H-NMR (400 MHz, d₆-DMSO) δ 10.22 (s, 1H), 9.79 (s, 1H), 9.61 (s, 1H), 8.21 (s, 1H), 8.12 (d, J=5.3 Hz, 1H), 8.07 (d, J=7.7 Hz, 1H), 8.00 (d, J=2.1 Hz), 7.74-7.80 (m, 3H), 7.59-7.63 (m, 2H), 7.37-7.42 (m, 1H), 7.20 (t, J=8.1 Hz, 2H), 7.14 (d, J=9.0 Hz, 1H), 6.31 (d, J=5.5 Hz, 1H), 4.35 (t, J=4.5 Hz, 2H), 3.55 (t, J=3.5 Hz, 2H), and 2.92 (s, 6 H); MS (ESI) m/z=622 [M+H]⁺.

### Example 76

### 3-(2-Amino-5-{2-[(3-{[2-(dimethylamino)ethyl]oxy}phenyl)amino]-4-pyrimidinyl}-1,3-thiazol-4-yl)-N-(2,6-difluorophenyl)benzamide

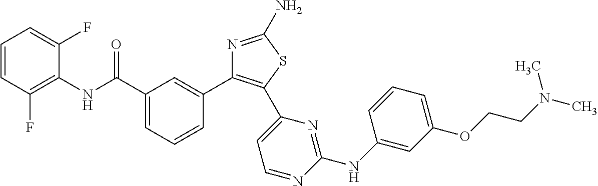

The title compound was prepared from 3-[2-amino-5-(2-chloro-4-pyrimidinyl)-1,3-thiazol-4-yl]-N-(2,6-difluorophenyl)benzamide (see Example 75, Step C) and {2-[(3-aminophenyl)oxy]ethyl}dimethylammonium chloride using displacement conditions described in Example 75, Step D to generate the title compound of Example 76 in 64% yield. ¹H-NMR (400 MHz, d₆-DMSO) δ 10.22 (s, 1H), 9.67 (s, 1H), 9.62 (s, 1 H), 8.20 (s, 1H), 8.13 (d, J=5.5 Hz, 1H), 8.08 (d, J=7.9 Hz, 1H), 7.75-7.80 (m, 2H), 7.63 (t, J=7.7 Hz, 2H), 7.54 (s, 1H), 7.34-7.42 (m, 2H), 7.18-7.23 (m, 3H), 6.60 (d, J=8.2 Hz, 1H), 6.32 (d, J=5.5 Hz, 1H), 4.31 (t, J=4.7 Hz, 2H), 3.54 (t, J=4.4 Hz, 2H), and 2.87 (s, 6H); MS (ESI) m/z=588 [M+H]⁺.

### Example 77

### 3-[2-Amino-5-(2-{[3-(1-pyrrolidinylmethyl)phenyl]amino}-4-pyrimidinyl)-1,3-thiazol-4-yl]-N-(2,6-difluorophenyl)benzamide

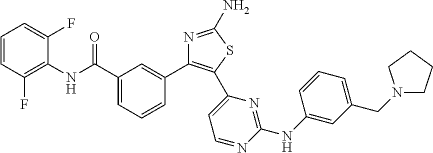

The title compound of Example 77 was prepared from 3-[2-amino-5-(2-chloro-4-pyrimidinyl)-1,3-thiazol-4-yl]-N-(2,6-difluorophenyl)benzamide (see Example 75, Step C) and [3-(1-pyrrolidinylmethyl)phenyl]amineusing displacement conditions described in Example 75, Step D to generate the title compound in 44% yield. ¹H-NMR (400 MHz, d₆-DMSO) δ 10.22 (s, 1H), 9.80 (s, 1H), 9.71 (s, 1H), 8.20 (s, 1H), 8.15 (d, J=5.3 Hz, 1H), 8.07 (d, J=7.7 Hz, 1H), 7.87 (s, 1H), 7.74-7.80 (m, 3H), 7.62 (t, J=7.7 Hz, 1H), 7.54 (s, 1H), 7.33-7.43 (m, 2H), 7.21 (t, J=8.1 Hz, 3H), 7.09 (d, J=7.5 Hz, 1H), 6.34 (d, J=5.5 Hz, 1H), 4.31 (s, 2H), 3.38-3.42 (m, 2H), 3.09-3.13 (m, 2H), 2.01-2.05 (m, 2H), and 1.84-1.87 (m, 2H); MS (ESI) m/z=584 [M+H]⁺.

### Example 78

### N-{3-[5-(2-{[3-{[2-(Dimethylamino)ethyl]oxy}-4-(methyloxy)phenyl]amino}-4-pyrimidinyl)-1,3-thiazol-4-yl]phenyl}-2-(2-thienyl)acetamide

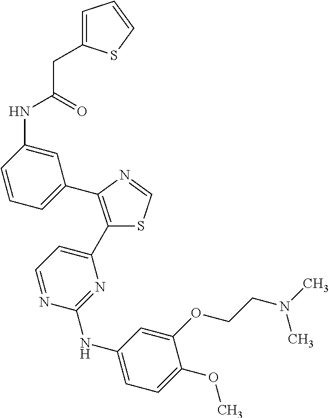

### Step A: 2-Chloro-4-[4-(3-nitrophenyl)-1,3-thiazol-5-yl]pyrimidine

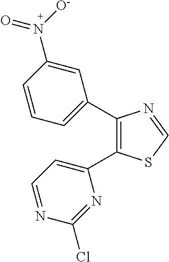

5-(2-Chloro-4-pyrimidinyl)-4-(3-nitrophenyl)-1,3-thiazol-2-amine (1.80 g, 5.39 mmol), prepared in a procedure analogous to Example 64, Step B, was dissolved in 25 mL of EtOAc and heated to 50° C. T-butyl nitrite (0.961 mL, 8.09 mmol) was added while stirring at 50° C. for 3 h. The reaction was cooled to rt, diluted with EtOAc (100 mL), and washed with H₂O (3×100 mL). The organic layers were combined, dried over MgSO₄, loaded onto silica, and purified via silica chromatography, eluting with an EtOAc/Hexanes mixture. The desired fractions were combined and the solvent removed to yield 1.50 g (87%) of 2-chloro-4-[4-(3-nitrophenyl)-1,3-thiazol-5-yl]pyrimidine. ¹H-NMR (400 MHz, DMSO-D₆) δ 9.4 (s, 1H), 8.6 (d, J=5.1 Hz, 1H), 8.4 (s, 1H), 8.3 (dt, J=8.3 and 1.2 Hz, 1H), 8.0 (m, 1H), 7.7 (t, J=8.0 Hz, 1H), and 7.4 (d, J=5.5 Hz, 1H); ESIMS m/z 319 (M+H).

### Step B: 3-[5-(2-Chloro-4-pyrimidinyl)-1,3-thiazol-4-yl]phenyl amine

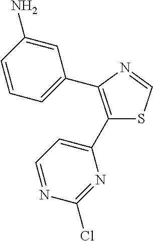

A solution of 2-chloro-4-[4-(3-nitrophenyl)-1,3-thiazol-5-yl]pyrimidine (1.50 g, 4.49 mmol) and 10% Pd/C (0.800 g) in 50 mL of ETOAc was treated with 45 psi of H₂ overnight. The reaction was filtered through celite and the solvent was removed to give 1.00 g (77% yield) of 3-[5-(2-chloro-4-pyrimidinyl)-1,3-thiazol-4-yl]phenyl amine as a yellow solid. ¹H-NMR (400 MHz, DMSO-D₆) δ 9.3 (s, 1H), 8.6 (d, J=5.3 Hz, 1H), 7.2 (d, J=5.3 Hz, 1H), 7.1 (t, J=7.7 Hz, 1H), 6.7 (s, 1H), 6.8 (m, 2H), and 5.3 (s, 2H); ESIMS m/z 289 (M+H).

### Step C: N-{3-[5-(2-Chloro-4-pyrimidinyl)-1,3-thiazol-4-yl]phenyl}-2-(2-thienyl)acetamide

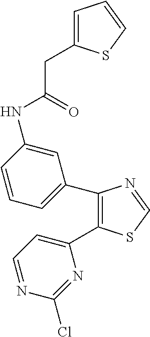

3-[5-(2-Chloro-4-pyrimidinyl)-1,3-thiazol-4-yl]phenyl amine (1.00 g, 3.46 mmol) was dissolved in 10 mL of THF and thiophene acetyl chloride (0.612 g, 3.81 mmol) was added while stirring at rt for 1 h. The solvent was removed in vacuo and the residue was taken up in 100 mL of EtOAc and washed with saturated NaHCO₃. The organic layers were dried over MgSO₄, loaded onto silica, and purified via silica chromatography, eluting with an EtOAc/Hexanes gradient to afford 1.23 g (86% yield) of N-{3-[5-(2-chloro-4-pyrimidinyl)-1,3-thiazol-4-yl]phenyl}-2-(2-thienyl)acetamide as a pale yellow solid. ¹H-NMR (400 MHz, DMSO-D₆) δ 10.3 (s, 1H), 9.4 (s, 1H), 8.6 (d, J=5.3 Hz, 1H), 7.8 (s, 1H), 7.7 (d, J=7.5 Hz, 1H), 7.4 (m, 2H), 7.2 (m, 2H), 7.0 (d, J=3.8 Hz, 2H), and 3.9 (s, 2H); ESIMS m/z 413 (M+H).

### Step D: N-{3-[5-(2-{[3-{[2-(Dimethylamino)ethyl]oxy}-4-(methyloxy)phenyl]amino}-4-pyrimidinyl)-1,3-thiazol-4-yl]phenyl}-2-(2-thienyl)acetamide

Title compound of Example 78 was synthesized using standard chloride displacement conditions analogous to Example 1, Step D, using N-{3-[5-(2-chloro-4-pyrimidinyl)-1,3-thiazol-4-yl]phenyl}-2-(2-thienyl)acetamide (0.080 g, 0.19 mmol) and 3-{[2-(dimethylamino)ethyl]oxy}-4-(methyloxy)phenyl amine (0.045 g, 0.21 mmol) in the microwave and purified by silica chromatography to give 0.023 g, (22% yield) of the title compound of Example 78. ¹H-NMR (400 MHz, DMSO-D₆) δ 10.3 (s, 1H), 9.5 (s, 1H), 8.3 (d; J=5.3 Hz, 1H), 7.8 (s, 1H), 7.7 (dd, J=7.6 and 1.7 Hz, 1H), 7.5 (d, J=2.4 Hz, 1H), 7.4 (m, 2H), 7.2 (dd, J=15.1 and 8.7 Hz, 2H), 7.0 (d, J=4.2 Hz, 2H), 6.9 (d, J=8.8 Hz, 1H), 6.5 (d, J=5.1 Hz, 1H), 4.0 (t, J=6.0 Hz, 2H), 3.9 (s, 2H), 3.7 (s, 3H), 2.6 (t, J=6.1 Hz, 2H), and 2.2 (s, 6H); ESIMS m/z 587 (M+H).

### Example 79

### N-[3-(5-{2-[(3-{[2-(Dimethylamino)ethyl]oxy}phenyl)amino]-4-pyrimidinyl}-1,3-thiazol-4-yl)phenyl]-2,6-difluorobenzamide-formic acid

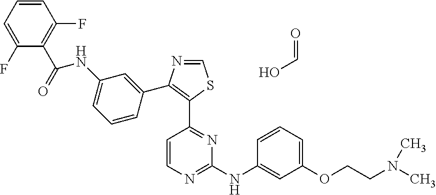

N-{3-[5-(2-Chloro-4-pyrimidinyl)-1,3-thiazol-4-yl]phenyl}-2,6-difluorobenzamide

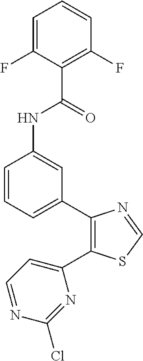

Step A: To a solution containing 0.365 g (1.26 mmol) of {3-[5-(2-chloro-4-pyrimidinyl)-1,3-thiazol-4-yl]phenyl}amine, prepared in a procedure analogous to Example 64, Step C and 30 mL of THF was added 0.19 mL (1.52 mmol) of 2,6-difluorobenzoyl chloride. The reaction was allowed to stir for 30 min, and then TEA was added. The solvents were removed under reduced pressure and the residue was subjected to silica gel chromatography to give 340 mg (63%) of N-{3-[5-(2-chloro-4-pyrimidinyl)-1,3-thiazol-4-yl]phenyl}-2,6-difluorobenzamide: ESI MS: 429.05 (M+H⁺).

### Step B: N-[3-(5-{2-[(3-{[2-(Dimethylamino)ethyl]oxy}phenypamino]-4-pyrimidinyl}-1,3-thiazol-4-yl)phenyl]-2,6-difluorobenzamide-formic acid

The title compound of Example 79 was prepared from N-{3-[5-(2-chloro-4-pyrimidinyl)-1,3-thiazol-4-yl]phenyl}-2,6-difluorobenzamide (0.07 g, 0.16 mmol) and 3-{[2-(dimethylamino)ethyl]oxy}phenyl amine (0.03 g, 0.16 mmol) by a procedure analogous to Example 1, Step D, and purified using HPLC to yield 35 mg. ¹H-NMR (300 MHz, DMSO-d₆) δ 10.92 (s, 1H), 9.74 (s, 1H), 9.31 (s, 1H), 8.37 (d, J=5.2 Hz, 1H), 7.96 (s, 1H), 7.79 (d, J=7.9 Hz, 1H), 7.58 (m, 1H), 7.51-7.42 (m, 2H), 7.33 (d, J=7.8 Hz, 1H), 7.31-7.19 (m, 3H), 7.15 (t, J=8.2 Hz, 1H), 6.62 (d, J=5.0 Hz, 1H), 6.54 (dd, J=7.9 and 2.4 Hz, 1H), 4.04 (t, J=6.0 Hz, 2H), 2.64 (t, J=6.1 Hz, 2H), and 2.22 (s, 6H); HRMS C₃₀H₂₇F₂N₆O₂S (M+H)⁺ calcd 573.1879. found 573.1876.

### Example 80

### 2,6-Difluoro-N-[3-(5-{2-[(3-fluoro-4-{4-[2-(methylsulfonyl)ethyl]-1-piperazinyl}phenyl)amino]-4-pyrimidinyl}-1,3-thiazol-4-yl)phenyl]benzamide

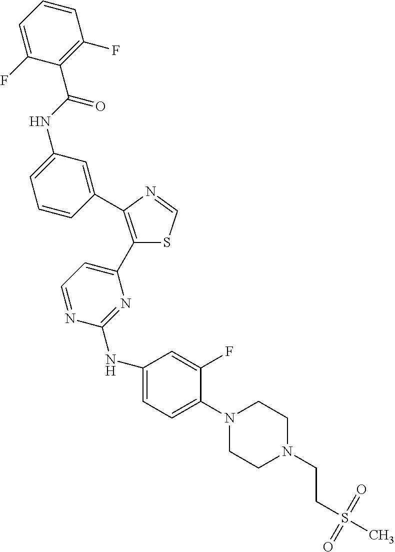

### Step A: 1-(2-Fluoro-4-nitrophenyl)-4-[2-(methylsulfonyl)ethyl]piperazine

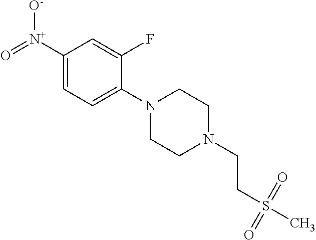

To a solution of 1-(2-fluoro-4-nitrophenyl)piperazine (0.50 g, 2.22 mmol) in i-PrOH (25 mL), methyl vinyl sulfone (0.354 g, 3.33 mmol) was added. The reaction was heated to reflux for 18 h. The reaction was cooled to rt, loaded directly onto silica, and purified via flash chromatography EtOAC/EtOAc:MeOH:NH₄OH (80:19:1) 0-100% gradient over 15 min. The desired fractions were combined and the solvent was removed to give 0.500 g of a yellow solid. ¹H-NMR (400 MHz, DMSO-d₆) δ 7.93-8.00 (m, 2H), 7.13 (td, J=9.1 and 0.7 Hz, 1H), 3.22-3.31 (m, 6H), 3.00 (s, 3H), 2.73 (t, J=6.6 Hz, 2H), and 2.56 (brs, 4H).

### Step B: 3-Fluoro-4-{4-[2-(methylsulfonyl)ethyl]-1-piperazinyl}phenyl amine

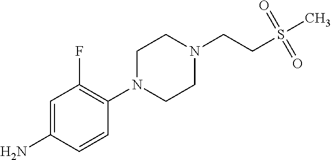

1-(2-Fluoro-4-nitrophenyl)-4-[2-(methylsulfonyl)ethyl]piperazine (0.500 g, 1.51 mmol) was taken up in EtOH (15 mL) and 10% Palladium/Carbon (0.050 g) was added. The mixture was stirred under H₂, 60 psi, for 3 h. The reaction was filtered through a celite plug and the solvent was removed to give 0.450 g, 98% yield, of a yellow solid. ¹H-NMR (400 MHz, DMSO-d₆) δ 6.67-6.74 (m, 1H), 6.21-6.31 (m, 2H), 4.92 (s, 2H), 3.22-3.28 (m, 3H), 2.97-3.01 (m, 3H), 2.77 (brs, 4H), 2.66-2.72 (m, 2H), and 2.50 (brs, 3H).

### Step C: 2,6-Difluoro-N-[3-(5-{2-[(3-fluoro-4-{4-[2-(methylsulfonyl)ethyl]-1-piperazinyl}phenyl)amino]-4-pyrimidinyl}-1,3-thiazol-4-yl)phenyl]benzamide

The title compound of Example 80 was synthesized using standard microwave chloride displacement conditions analogous to Example 1, Step D, in trifluoroethanol using N-{3-[5-(2-chloro-4-pyrimidinyl)-1,3-thiazol-4-yl]phenyl}-2,6-difluorobenzamide (0.080 g, 0.19 mmol), prepared by a procedure analogous to Example 79, Step A, and 3-fluoro-4-{4-[2-(methylsulfonyl)ethyl]-1-piperazinyl}phenyl amine (0.062 g, 0.21 mmol). The solvent was removed and the residue was taken up in DMSO/MeOH (2:1) and purified via HPLC. The desired fractions were combined, diluted with EtOAc, and washed with NaHCO₃ x₂. The organic layer was dried over MgSO₄ and the solvent was removed to give 0.037 g, 28% yield, of desired product as a yellow solid. ¹H-NMR (400 MHz, DMSO-d₆) δ 10.90 (s, 1H), 9.77 (s, 1H), 8.34 (d, J=5.3 Hz, 1H), 7.94 (s, 1H), 7.76 (d, J=7.7 Hz, 1H), 7.62 (d, J=2-0.4 Hz, 2H), 7.54 (d, 1H), 7.43 (t, J=7.9 Hz, 1H), 7.35 (dd, J=8.6 and 2.0 Hz, 1H), 7.28 (s, 1H), 7.22 (t, J=7.9 Hz, 2H), 6.93 (t, J=9.4 Hz, 1H), 6.60 (d, J=5.1 Hz, 1H), 3.48 (s, 2H), 3.02 (s, 3H), 2.93 (s, 4H), 2.74 (t, J=6.6 Hz, 2H), and 2.56 (s, 4H); ES-LCMS m/z 694 (M+H).

M+H).

### Example 81

### N-{3-[5-(2-{[3-(1-Pyrrolidinylmethyl)phenyl]amino}-4-pyrimidinyl)-1,3-thiazol-4-yl]phenyl}-2-(2-thienyl)acetamide

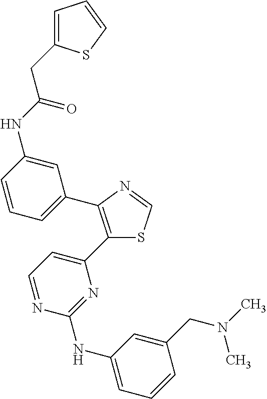

Title compound of Example 81 was synthesized using standard chloride displacement conditions analogous to Example 1, Step D, using N-{3-[5-(2-chloro-4-pyrimidinyl)-1,3-thiazol-4-yl]phenyl}-2-(2-thienyl)acetamide (0.08 g, 0.19 mmol), prepared by a procedure analogous to Example 78, Step E, and 3-[(dimethylamino)methyl]phenyl amine (0.032 g, 0.21 mmol) in the microwave, and purified by silica chromatography to give 0.026 g, (26% yield) of the title compound of Example 81. ¹H-NMR (400 MHz, DMSO-D₆) δ 10.3 (s, 1H), 9.7 (s, 1H), 9.3 (s, 1H), 8.3 (d, J=5.3 Hz, 1H), 7.8 (s, 1H), 7.8 (s, 1H), 7.7 (d, J=8.2 Hz, 1H), 7.6 (d, J=7.9 Hz, 1H), 7.4 (m, 2H), 7.2 (m, 2H), 7.0 (d, J=4.4 Hz, 2H), 6.9 (d, J=7.3 Hz, 1H), 6.6 (d, J=5.1 Hz, 1H), 3.9 (s, 2H), 3.4 (s, 2H), and 2.1 (s, 6H); ESIMS m/z 527 (M+H).

### Example 82

### N-[3-(5-{2-[(3-{[2-(Diethylamino)ethyl]oxy}phenyl)amino]-4-pyrimidinyl}-1,3-thiazol-4-yl)phenyl]-2-(2-thienyl)acetamide

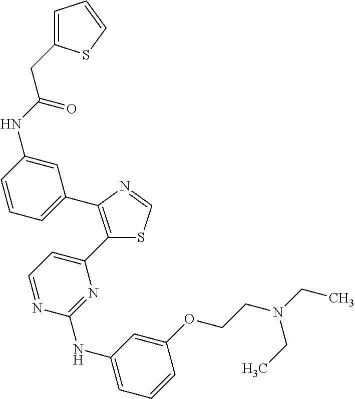

Title compound of Example 82 was synthesized using standard chloride displacement conditions analogous to Example 1, Step D, using N-{3-[5-(2-chloro-4-pyrimidinyl)-1,3-thiazol-4-yl]phenyl}-2-(2-thienyl)acetamide (0.080 g, 0.19 mmol), prepared by a procedure analogous to Example 78, Step E, and 3-{[2-(diethylamino)ethyl]oxy}phenyl amine (0.044 g, 0.21 mmol) in the microwave and purified by silica chromatography to give 0.034 g, (30% yield) of the title compound of Example 82. ¹H-NMR (400 MHz, DMSO-D₆) δ 10.3 (s, 1H), 9.7 (s, 1H), 8.3 (d, J=5.1 Hz, 1H), 7.8 (s, 1H), 7.7 (d, J=8.4 Hz, 1H), 7.5 (s, 1H), 7.4 (m, 2H), 7.3 (m, 2H), 7.1 (t, J=8.1 Hz, 1H), 7.0 (s, 2H), 6.5 (m, 2H), 4.0 (t, J=6.3 Hz, 2H), 3.9 (s, 2 H), 2.8 (t, J=6.1 Hz, 2H), 2.5 (q, J=7.0 Hz, 4H), and 1.0 (t, J=7.0 Hz, 6H); ESIMS m/z 585 (M+H).

### Example 83

### N-{3-[5-(2-{[2-(Dimethylamino)-2,3-dihydro-1H-inden-5-yl]amino}-4-pyrimidinyl)-1,3-thiazol-4-yl]phenyl}-2-(2-thienyl)acetamide

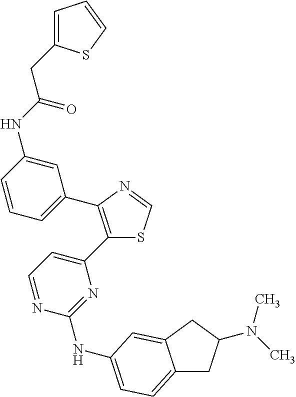

Title compound of Example 83 was synthesized using standard chloride displacement conditions analogous to Example 1, Step D, using N-{3-[5-(2-chloro-4-pyrimidinyl)-1,3-thiazol-4-yl]phenyl}-2-(2-thienyl)acetamide (0.080 g, 0.19 mmol), prepared by a procedure analogous to Example 78, Step E, and (5-amino-2,3-dihydro-1H-inden-2-yl)dimethylamine (0.038 g, 0.21 mmol), prepared in a procedure analogous to Example 12, Step B, in the microwave and purified by silica chromatography to give 0.029 g, (27% yield) of the title compound of Example 83. ¹H-NMR (400 MHz, DMSO-D₆) δ 10.3 (s, 1H), 9.6 (s, 1H), 9.3 (m, 1H), 8.3 (d, J=5.1 Hz, 1H), 7.8 (s, 1H), 7.7 (d, J=8.1 Hz, 1H), 7.6 (s, 1H), 7.4 (m, 3H), 7.2 (d, J=7.7 Hz, 1H), 7.1 (d, J=8.2 Hz, 1H), 7.0 (s, 2H), 6.5 (d, J=5.1 Hz, 1H), 3.9 (s, 2H), 2.9 (m, 2H), 2.7 (m, 2H), and 2.2 (s, 6H); ESIMS m/z 553 (M+H).

### Example 84

### N-[3-(5-{2-[(3-{[2-(Dimethylamino)ethyl]oxy}phenyl)amino]-4-pyrimidinyl}-1,3-thiazol-4-yl)phenyl]-2-(2-thienyl)acetamide

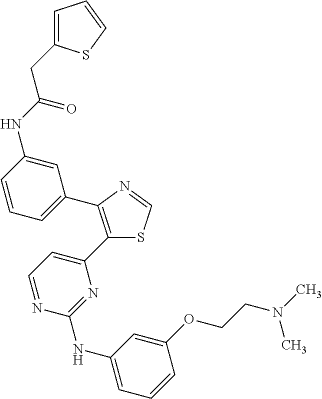

Title compound of Example 82 was synthesized using standard chloride displacement conditions analogous to Example 1, Step D, using N-{3-[5-(2-chloro-4-pyrimidinyl)-1,3-thiazol-4-yl]phenyl}-2-(2-thienyl)acetamide (0.08 g, 0.19 mmol), prepared by a procedure analogous to Example 78, Step E, and {2-[(3-aminophenyl)oxy]ethyl}dimethylamine hydrochloride (0.044 g, 0.21 mmol) in the microwave and purified by silica chromatography to give 0.013 g, (12% yield) of the title compound of Example 84. ¹H-NMR (400 MHz, DMSO-D₆) δ 10.3 (s, 1H), 9.7 (s, 1H), 8.3 (d, J=4.9 Hz, 1H), 7.8 (s, 1H), 7.7 (d, J=7.5 Hz, 1H), 7.5 (s, 1H), 7.4 (m, 2H), 7.3 (dd, J=17.9 and 7.8 Hz, 2H), 7.2 (t, J=8.1 Hz, 1H), 7.0 (s, 2H), 6.6 (m, 2H), 4.0 (t, J=6.0 Hz, 2H), 3.9 (s, 2H), 2.6 (t, J=5.8 Hz, 2H), and 2.2 (s, 6H); ESIMS m/z 557 (M+H).

### Example 85

### N-[3-(5-{2-[(3-Oxo-3,4-dihydro-2H-1,4-benzoxazin-7-yl)amino]-4-pyrimidinyl}-1,3-thiazol-4-yl)phenyl]-2-(2-thienyl)acetamide hydrochloride

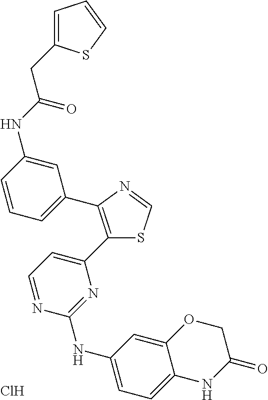

Title compound of Example 85 was synthesized using standard chloride displacement conditions analogous to Example 1, Step D, using N-{3-[5-(2-chloro-4-pyrimidinyl)-1,3-thiazol-4-yl]phenyl}-2-(2-thienyl)acetamide (0.08 g, 0.19 mmol), prepared by a procedure analogous to Example 78, Step E, and 7-amino-2H-1,4-benzoxazin-3(4H)-one (0.044 g, 0.21 mmol) in the microwave and solid precipitate was filtered and rinsed with ether to give 0.058 g, (53% yield) of the title compound of Example 85. ¹H-NMR (400 MHz, DMSO-D₆) δ 10.6 (s, 1H), 10.3 (s, 1H), 9.7 (s, 1H), 8.3 (d, J=5.7 Hz, 1H), 7.8 (s, 1H), 7.7 (d, J=7.9 Hz, 1H), 7.5 (s, 1H), 7.4 (m, 2H), 7.3 (dd, J=15.5 and 7.6 Hz, 2H), 7.0 (s, 2H), 6.8 (d, J=9.2 Hz, 1H), 6.5 (d, J=4.8 Hz, 1H), 4.5 (s, 2H), and 3.9 (s, 2H); ESMIS m/z 541 (M+H).

### Example 86

### N-[3-(5-{2-[(3-Oxo-3,4-dihydro-2H-1,4-benzoxaziN-6-yl)amino]-4-pyrimidinyl}-1,3-thiazol-4-yl)phenyl]-2-(2-thienyl)acetamide hydrochloride

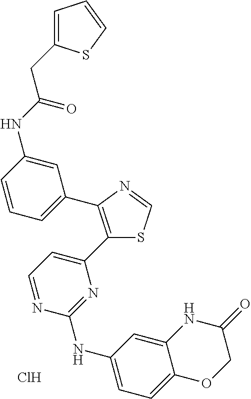

Title compound of Example 86 was synthesized using standard chloride displacement conditions analogous to Example 1, Step D, using N-{3-[5-(2-chloro-4-pyrimidinyl)-1,3-thiazol-4-yl]phenyl}-2-(2-thienyl)acetamide (0.08 g, 0.19 mmol), prepared by a procedure analogous to Example 78, Step E, and 6-amino-2H-1,4-benzoxazin-3(4H)-one (0.044 g, 0.21 mmol) in the microwave and solid precipitate was filtered and rinsed with ether to give 0.065 g, (59% yield) of the title compound of Example 86. ¹H-NMR (400 MHz, DMSO-D₆) δ 10.7 (s, 1H), 10.3 (s, 1H), 9.7 (s, 1H), 8.3 (d, J=4.6 Hz, 1H), 7.8 (s, 1H), 7.7 (d, J=7.9 Hz, 1H), 7.4 (d, J=11.0 Hz, 3H), 7.2 (m, 2H), 6.9 (s, 1H), 6.8 (d, J=8.8 Hz, 1H), 6.5 (d, J=4.6 Hz, 1H), 4.5 (s,

### Example 87

### 2,6-Difluoro-N-{3-[5-(2-{[3-fluoro-4-(methyloxy)phenyl]amino}-4-pyrimidinyl)-1,3-thiazol-4-yl]phenyl}benzamide

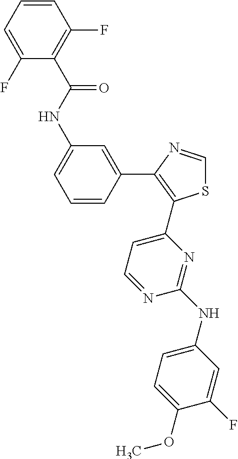

### Step A: 2-[2-(Methylthio)-4-pyrimidinyl]-1-(3-nitrophenyl)ethanone

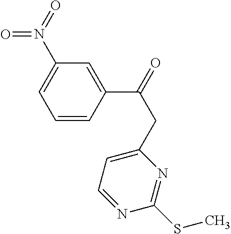

To a solution containing 10 g (71 mmol) of 4-methyl-2-(methylthio)pyrimidine, 12.9 g (71 mmol) of m-nitromethylbenzoate, and 65 mL of THF at 0° C. was slowly added 142 mL (142 mmol) of a 1.0 M solution of LHMDS in hexanes. The reaction mixture was allowed to stir at 0° C. for 12 h, then quenched by the addition of 2.0 M aqueous HCl. After 3 h, the resulting mixture was filtered and the filtercake was collected and recrystallized from EtOH to give 8.9 g (43%) of the title compound of Step A as a yellow solid: ¹H-NMR (CDCl₃, 400 MHz) δ 8.66 (s, 1H), 8.38 (d, 1H, J=5.5 Hz), 8.27 (d, 1H, J=8.1 Hz), 8.15 (d, 1H, J=7.7 Hz), 7.59 (t, 1H, J=7.8 Hz), 6.71 (d, 1H, J=5.3 Hz), 6.07 (s, 1H), and 2.62 (s, 3H).

### Step B: 5-[2-(Methylthio)-4-pyrimidinyl]-4-(3-nitrophenyl)-1,3-thiazol-2-amine

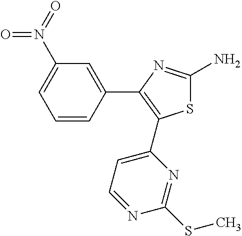

To a solution containing 3.0 g (10.4 mmol) of 2-[2-(methylthio)-4-pyrimidinyl]-1-(3-nitrophenyl)ethanone and 200 mL of HOAc was added 0.53 mL (10.4 mmol) of bromine dropwise. The reaction mixture was allowed to stir for 2 h and the HOAc was removed under reduced pressure. The residue was partitioned between DCM and aqueous NaHCO₃. The organic layers were collected and dried over MgSO₄. The solvents were removed under reduced pressure to give 4-[2-bromo-4-(3-nitrophenyl)-1,3-thiazol-5-yl]-2-(methylthio)pyrimidine as a thick orange oil. A solution containing the crude bromide, 100 mL of dioxane, 4.2 g of MgCO₃ and 4.2 g of thiourea was heated at 80° C. for 2.5 h. The yellow reaction mixture was partitioned between DCM and water. The combined organic layers were dried over MgSO₄ and the solvent was removed under reduced pressure. The residue was subjected to silica gel chromatography to give 0.85 g (24%) of the title compound of Step B as an orange solid: ¹H-NMR (CDCl₃, 400 MHz) δ 8.47 (s, 1H), 8.29 (d, 1H, J=7.4 Hz), 8.19 (d, 1H, J=5.4 Hz), 7.88 (d, 1H, J=7.5 Hz), 7.61 (t, 1H, J=8.3 Hz), 6.59 (d, 1H, J=5.9 Hz), 5.25 (brs, 2H), and 2.50 (s, 3H).

### Step C: 2-(Methylthio)-4-[4-(3-nitrophenyl)-1,3-thiazol-5-yl]pyrimidine

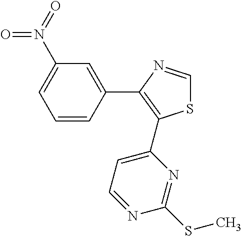

A solution containing 0.85 g (2.46 mmol) of 5-[2-(methylthio)-4-pyrimidinyl]-4-(3-nitrophenyl)-1,3-thiazol-2-amine, 0.44 mL (3.70 mmol) of nitrite, and 20 mL of THF was heated at 50° C. for 4 h. The reaction mixture was diluted in EtOAc and washed with H₂O. The organic layers were dried over MgSO₄ and the solvent was removed under reduced pressure to give 0.80 g (99%) of the title compound of Step C as a viscous yellow oil: ¹H-NMR (CDCl₃, 400 MHz) δ 8.97 (s, 1H), 8.53 (t, 1H), 8.37 (d, 1H, J=5.2 Hz), 8.31 (d, 1H, J=8.2 Hz), 7.92 (d, 1H, J=7.8 Hz), 7.63 (t, 1H, J=7.7 Hz), 6.81 (d, 1H, J=5.2 Hz), and 2.52 (s, 3H); ESIMS: 331.0 (M+H⁺).

### Step D: (3-{5-[2-(Methylthio)-4-pyrimidinyl]-1,3-thiazol-4-yl}phenyl)amine

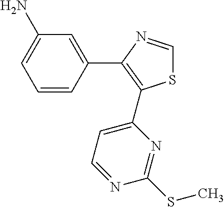

A solution containing 0.80 g (2.42 mmol) of 2-(methylthio)-4-[4-(3-nitrophenyl)-1,3-thiazol-5-yl]pyrimidine, 2.91 g (12.11 mmol) of sodium sulfide nonahydrate, 10 mL of EtOHI, and 10 mL of H₂O was heated at 50° C. for 3 h. The EtOH was removed under reduced pressure and the reaction mixture was extracted with EtOAc. The organic layers were dried over MgSO₄ and the solvent was removed under reduced pressure to give 0.52 g (72%) of the title compound of Step D as a viscous brown oil: ¹H-NMR (CDCl₃, 400 MHz) δ 8.90 (s, 1H), 8.30 (d, 1H, J=5.2 Hz), 7.22 (t, 1H, J=8.1 Hz), 6.88-6.90 (m, 3H), 6.77 (d, 1H, J=9.2 Hz), 3.76 (brs, 2H), and 2.58 (s, 3H); ESIMS: 301.0 (M+H⁺).

### Step E: 2,6-Difluoro-N-(3-{5-[2-(methylthio)-4-pyrimidinyl]-1,3-thiazol-4-yl}phenyl)benzamide

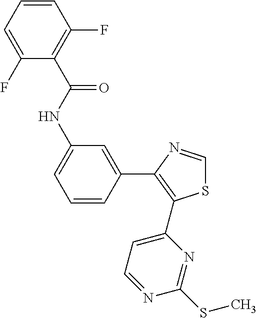

To a solution containing 260 mg (0.86 mmol) of (3-{5-[2-(methylthio)-4-pyrimidinyl]-1,3-thiazol-4-yl}phenyl)amine and 5 mL of DCM was added 0.12 mL (0.95 mmol) of 2,6-difluorobenzoyl chloride, followed by 0.18 mL (1.29 mmol) of TEA. The reaction mixture was allowed to stir at rt for 12 h, then diluted in DCM and washed with aqueous ammonium chloride and aqueous NaHCO₃. The organic layers were dried over MgSO₄, the solvent was removed under reduced pressure, and the residue was subjected to silica gel chromatography to give 178 mg (47%) of the title compound of Step E as a yellow solid: ESIMS: 441.0 (M+H⁺).

### Step F: 2,6-Difluoro-N-{3-[5-(2-{[3-fluoro-4-(methyloxy)phenyl]amino}-4-pyrimidinyl)-1,3-thiazol-4-yl]phenyl}benzamide

To a solution containing 178 mg (0.4 mmol) of 2,6-difluoro-N-(3-{5-[2-(methylthio)-4-pyrimidinyl]-1,3-thiazol-4-yl}phenyl)benzamide and 5 mL of DCM was added 140 mg of (0.8 mmol) of meta-chloroperbenzoic acid. The reaction mixture was allowed to stir at rt for 2.5 h, then diluted with DCM and washed with H₂O and aqueous NaHCO₃ to give 150 mg of a complex mixture of oxidation products which was used without further purification. To a solution containing 75 mg of the above oxidation mixture and 1.0 mL DMA was added 71 mg (0.5 mmol) of 3-fluoro-4-methoxyphenyl amine and 1 drop of conc HCl. The reaction mixture was heated at 150° C. for 5 h, then cooled and partitioned between DCM and water. The organic layers were dried over MgSO₄ and the solvent was removed under reduced pressure. The residue was subjected to silica gel chromatography to give 30 mg of the title compound of Example 87 as a yellow solid: ¹H-NMR (d⁶-DMSO, 400 MHz) δ 9.31 (s, 1H), 8.40 (d, 1H, J=5.1 Hz), 8.01 (s, 1H), 7.79-7.85 (m, 2H), 7.47 (d, 1H, J=8.2 Hz), 7.40 (t, 1H, J=7.9 Hz), 7.28 (dd, 1H, J=15.4 and 8.1 Hz), 7.22 (d, 1H, J=7.7 Hz), 6.99-7.03 (m, 2H), 6.76 (td, 2H, J=8.4 and 2.4 Hz), 6.65 (d, 1H, J=5.1 Hz), 6.06 (t, 1H, J=2.6 Hz), and 3.84 (s, 3H); APIMS: 533.1.

### Example 88

### 2,6-Difluoro-N-[3-(5-{2-[3-fluorophenyl)amino]-4-pyrimidinyl}-1,3-thiazol-4-yl)phenyl]benzamide

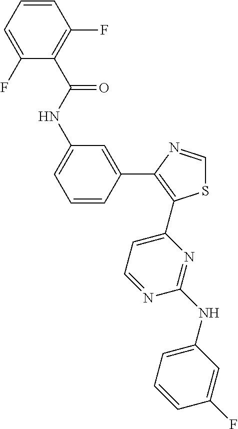

In a procedure analogous to Example 1, Step D, 25 mg of the title compound of Example 86 was synthesized as an off-white solid using 75 mg of the oxidation mixture from Example 87, Step F and 48 mg of 3-fluorophenyl amine: ¹H-NMR (CD₃OD, 400 MHz) δ 9.13 (s, 1H), 8.25 (d, 1H, J=5.2 Hz), 7.93 (t, 1H, J=1.5 Hz), 7.82 (d, 1H, J=8.3 Hz), 7.72 (dt, 1H, J=12.1 and 2.5 Hz), 7.46-7.52 (m, 2H), 7.35 (m, 1H), 7.24 (ddd, 1H, J=14.7, 7.8, and 6.5 Hz), 7.06-7.10 (m, 2H), 6.68 (td, 2H, J=8.4 and 1.5 Hz), and 6.65 (d, 1H, J=5.3 Hz); ESIMS: 504.1 (M+H⁺).

### Example 89

### N-[3-(5-{2-[3-Chloro-4-{[2-(dimethylamino)ethyl]oxy}phenyl)amino]-4-pyrimidinyl}-1,3-thiazol-4-yl)phenyl]-2,6-difluorobenzamide-formic acid

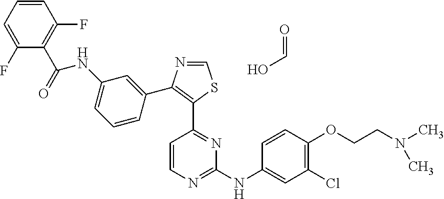

The title compound of Example 87 was prepared from N-{3-[5-(2-chloro-4-pyrimidinyl)-1,3-thiazol-4-yl]phenyl}-2,6-difluorobenzamide (0.05 g, 0.12 mmol), prepared by a procedure analogous to Example 79, Step A, and {2-[(4-amino-2-chlorophenypoxy]ethyl}dimethylamine dihydrochloride (0.034 g, 0.12 mmol), by a procedure analogous to Example 1, Step D, and purified using HPLC to yield 35 mg (46%). ¹H-NMR (300 MHz, DMSO-d₆) δ 10.91 (s, 1H), 9.75 (s, 1H), 9.32 (s, 1H), 8.36 (d, J=5.2 Hz, 1H), 7.96 (s, 1H), 7.93 (d, J=2.5 Hz, 1H), 7.78 (d, J=8.1 Hz, 1H), 7.64-7.50 (m, 2H), 7.46 (t, J=7.9 Hz, 1H), 7.32 (d, J=7.5 Hz, 1H), 7.24 (t, J=8.0 Hz, 2H), 7.08 (d, J=9.1 Hz, 1H), 6.61 (d, J=5.2 Hz, 1H), 4.08 (t, J=5.8 Hz, 2 H), 2.65 (t, J=5.6 Hz, 2H), and 2.24 (s, 6H); HRMS C₃₀H₂₆ClF₂N₆O₂S (M+H)⁺ calcd 607.1489. found 607.1488.

### Example 90

### N-(3-{5-[2-({3-[(Dimethylamino)methyl]-phenyl}amino)-4-pyrimidinyl]-1,3-thiazol-4-yl}phenyl)-2,6-difluorobenzamide-formic acid

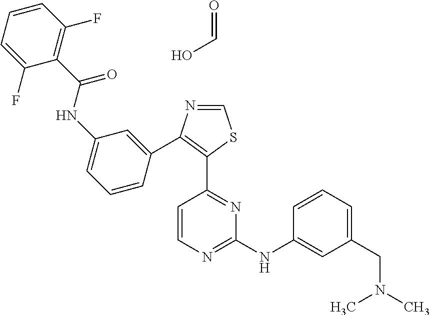

The title compound of Example 90 was prepared from N-{3-[5-(2-chloro-4-pyrimidinyl)-1,3-thiazol-4-yl]phenyl}-2,6-difluorobenzamide (0.07 g, 0.16 mmol), prepared by a procedure analogous to Example 79, Step A, and 3-[(dimethylamino)methyl]phenyl amine (0.025 g, 0.16 mmol), by a procedure analogous to Example 1, Step D, and purified using HPLC to yield 27 mg (28%). ¹H-NMR (400 MHz, DMSO-d₆) δ 10.92 (s, 1H), 9.73 (s, 1H), 9.31 (s, 1H), 8.35 (d, J=5.2 Hz, 1H), 7.96 (s, 1H), 7.78 (d, J=8.4 Hz, 1H), 7.74 (s, 1H), 7.63-7.54 (m, 2H), 7.46 (t, J=7.8 Hz, 1H), 7.32 (d, J=7.7 Hz, 1H), 7.28-7.16 (m, 3H), 6.88 (d, J=7.7 Hz, 1H), 6.61 (d, J=5.2 Hz, 1H), 3.35 (s, 2H), and 2.15 (s, 6H); MS (ESI) m/z 543 (M+H)⁺.

### Example 91

### 2,6-Difluoro-N-(3-{5-[2-(1,2,3,4-tetrahydro-7-isoquinolinylamino)-4-pyrimidinyl]-1,3-thiazol-4-yl}phenyl)benzamide

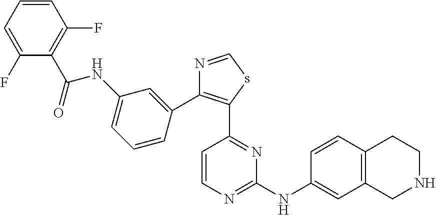

The title compound of Example 91 was prepared from N-{3-[5-(2-chloro-4-pyrimidinyl)-1,3-thiazol-4-yl]phenyl}-2,6-difluorobenzamide (0.07 g, 0.16 mmol), prepared by a procedure analogous to Example 79, Step A, and 2-(trifluoroacetyl)-1,2,3,4-tetrahydro-7-isoquinolinamine hydrochloride (0.04 g, 0.16 mmol), by a procedure analogous to Example 1, Step D. The material obtained, 2,6-difluoro-N-{3-[5-(2-{[2-(trifluoroacetyl)-1,2,3,4-tetrahydro-7-isoquinolinyl]amino}-4-pyrimidinyl)-1,3-thiazol-4-yl]phenyl}benzamide, MS (ESI) m/z 637 (M+H)⁺, was dissolved in a 3:1 mixture THF:H₂O (4 mL). LiOH (50 mg) was then added and the resulting mixture was heated at 50° C. until by LC/MS the TFA was removed from all of the starting material. The reaction was then diluted with EtOAc and the resulting organics were washed with H₂O and brine. The organics were dried over Na₂SO₄ and concentrated to yield solids which were then taken up in ether, sonicated, and filtered to yield 41 mg (47%) of the title compound of Example 91: ¹H-NMR (300 MHz, DMSO-d₆) δ 10.91 (s, 1H), 9.59 (s, 1H), 9.29 (s, 1H), 8.32 (d, J=5.1 Hz, 1H), 7.94 (s, 1H), 7.78 (d, J=8.3 Hz, 1H), 7.58 (m, 1H), 7.49-7.42 (m, 2H), 7.37-7.29 (m, 2H), 7.24 (t, J=8.0 Hz, 2H), 6.93 (d, J=8.4 Hz, 1H), 6.56 (d, J=5.2 Hz, 1H), 3.81 (s, 2H), 2.91 (t, J=5.7 Hz, 2H), and 2.60 (t, J=5.8 Hz, 2H); HRMS C₂₉H₂₃F₂N₆OS (M+H)⁺ calcd 541.1617. found 541.1613.

### Example 92

### N-[3-(5-{2-[(3-{[2-(Diethylamino)ethyl]oxy}phenyl)amino]-4-pyrimidinyl}-1,3-thiazol-4-yl)phenyl]-2,6-difluorobenzamide-formic acid

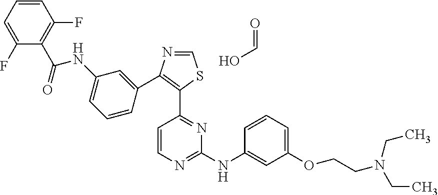

To obtain the title compound of Example 92, N-(3-{2-amino-5-[2-({3-[2-(diethylamino)ethoxy]phenyl}amino)pyrimidiN-4-yl]-1,3-thiazol-4-yl}phenyl)-2,6-difluorobenzamide (0.05 g, 0.081 mmol), prepared by a procedure analogous to Example 79, Step A, was dissolved in THF (3 mL). Tert-butyl nitrite (0.015 mL, 0.12 mmol)) was added drop-wise and the resulting mixture was heated at 50° C. for 3 h. The reaction was cooled to rt, adhered directly onto silica gel, and purified by silica gel chromatography using an EtOAc/MeOH/NH₄OH mixture. The mixture was further purified on a RP acidic HPLC. After concentrating fractions, 12 mg (23%) of the target compound was obtained. ¹H-NMR (300 MHz, DMSO-d₆) δ 10.93 (s, 1H), 9.74 (s, 1H), 9.30 (s, 1H), 8.37 (d, J=5.2 Hz, 1H), 7.96 (s, 1H), 7.79 (d, J=8.0 Hz, 1H), 7.58 (m, 1H), 7.54-7.43 (m, 2H), 7.33 (d, J=77 Hz, 1H), 7.30-7.19 (m, 3H), 7.15 (t, J=8.1 Hz, 1H), 6.62 (d, J=5.1 Hz, 1H), 6.53 (dd, J=8.1 and 2.2 Hz, 1H), 4.02 (t, J=6.3 Hz, 2H), 2.80 (t, J=6.2 Hz, 2H), 2.61-2.52 (m, 4H), and 0.97 (t, J=7.3 Hz, 6H); HRMS C₃₂H₃₁F₂N₆O₂S (M+H)⁺ calcd 601.2192. found 601.2188.

### Example 93

### 2,6-Difluoro-N-{3-[5-(2-{[4-(methyloxy)-3-(4-methyl-1-piperazinyl)phenyl]amino}-4-pyrimidinyl)-1,3-thiazol-4-yl]phenyl}benzamide

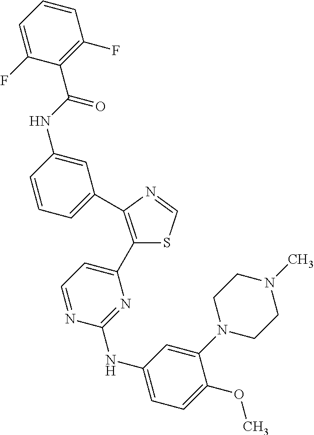

Title compound of Example 93 was synthesized using standard chloride displacement conditions in a procedure analogous to Example 1, Step D, using N-{3-[5-(2-chloro-4-pyrimidinyl)-1,3-thiazol-4-yl]phenyl}-2,6-difluorobenzamide (0.073 g, 0.17 mmol), prepared by a procedure analogous to Example 79, Step A, and 4-(methyloxy)-3-(4-methyl-1-piperazinyl)phenyl amine (0.041 g, 0.19 mmol) in the microwave and purified by silica chromatography to give 0.028 g, (27% yield) of the title compound of Example 91. ¹H-NMR (400 MHz, DMSO-D₆) δ 10.9 (s, 1H), 9.5 (s, 1H), 8.3 (d, J=5.3 Hz, 1H), 7.9 (s, 1H), 7.7 (d, J=8.6 Hz, 1H), 7.5 (t, J=8.5 Hz, 1H), 7.4 (t, J=7.9 Hz, 1H), 7.3 (m, 3H), 7.2 (t, J=8.0 Hz, 2H), 6.8 (d, J=8.8 Hz, 1H), 6.5 (d, J=5.3 Hz, 1H), 3.7 (s, 3H), 3.3 (s, 3H), and 2.5 (d, J=3.7 Hz, 8H); ESIMS m/z 614 (M+H).

### Example 94

### 2,6-Difluoro-N-{3-[5-(2-{[3-(1-pyrrolidinylmethyl)phenyl]amino}-4-pyrimidinyl)-1,3-thiazol-4-yl]phenyl}benzamide

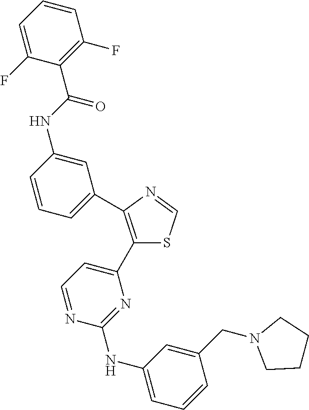

Title compound of Example 94 was synthesized using standard chloride displacement conditions in a procedure analogous to Example 1, Step D, using N-{3-[5-(2-chloro-4-pyrimidinyl)-1,3-thiazol-4-yl]phenyl}-2,6-difluorobenzamide (0.073 g, 0.17 mmol), prepared in a procedure analogous to Example 79, Step A, and 3-(1-pyrrolidinylmethyl)phenyl amine (0.033 g, 0.19 mmol) in the microwave and purified by silica chromatography to give 0.032 g, (32% yield) of the title compound of Example 94. ¹H-NMR (400 MHz, DMSO-D₆) δ 8.8 (s, 1H), 8.3 (s, 1H), 8.1 (d, J=5.1 Hz, 1H), 7.8 (d, J=8.2 Hz, 1H), 7.7 (s, 1H), 7.5 (m, 3H), 7.3 (m, 3H), 7.2 (d, J=5.3 Hz, 2H), 6.9 (d, J=7.5 Hz, 1H), 6.9 (t, J=8.1 Hz, 2H), 6.6 (d, J=5.3 Hz, 1H), 3.6 (s, 2H), 2.5 (s, 4H), and 1.7 (s, 4H); ESIMS m/z 569 (M+H).

### Example 95

### N-(3-{5-[2-({3-[2-(Aminosulfonyl)ethyl]phenyl}amino)-4-pyrimidinyl]-1,3-thiazol-4-yl}phenyl)-2,6-difluorobenzamide

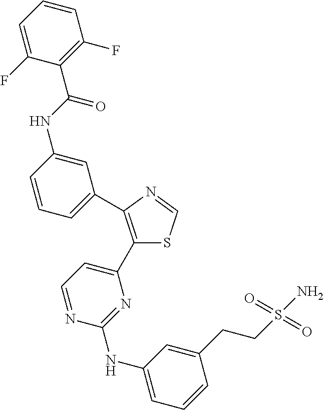

Title compound of Example 93 was synthesized using standard chloride displacement conditions in a procedure analogous to Example 1, Step D, using N-{3-[5-(2-chloro-4-py rimidinyl)-1,3-thiazol-4-yl]phenyl}-2,6-difluor obenzamide (0.073 g, 0.17 mmol), prepared by a procedure analogous to Example 79, Step A, and 2-(3-aminophenyl)ethanesulfonamide (0.038 g, 0.19 mmol), prepared by a procedure analogous to Example 8, Step A, in the microwave and purified by silica chromatography to give 0.028 g, (28% yield) of the title compound of Example 95. ¹H-NMR (400 MHz, DMSO-D₆) δ 8.9 (s, 1H), 8.2 (d, J=5.1 Hz, 1H), 7.8 (d, J=19.2 Hz, 3H), 7.8 (s, 1H), 7.4 (m, 4H), 7.3 (s, 3H), 7.0 (m, 2H), 6.9 (d, J=6.2 Hz, 1H), 6.7 (d, J=5.3 Hz, 1H), 3.5 (m, 2H), and 3.2 (m, 2H); ESIMS m/z 593 (M+H).

### Example 96

### N-[3-(5-{2-[(3-{[2-(Dimethylamino)ethyl]oxy}phenyl)amino]-4-pyrimidinyl}-1,3-thiazol-4-yl)phenyl]-2,5-difluorobenzamide

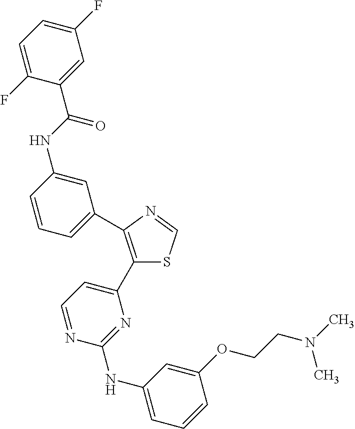

### Step A: N-{3-[5-(2-Chloro-4-pyrimidinyl)-1,3-thiazol-4-yl]phenyl}-2,5-difluorobenzamide

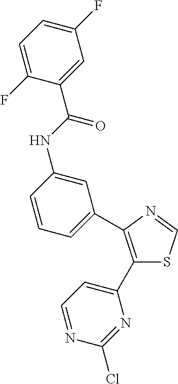

3-[5-(2-Chloro-4-pyrimidinyl)-1,3-thiazol-4-yl]phenyl amine (0.690 g, 2.39 mmol), prepared by a procedure analogous to Example 78, Step D, was dissolved in 10 mL of THF and 2,5-difluorobenzoyl chloride (0.463 g, 2.63 mmol) was added while stirring at rt for 1 hour. The THF was removed in vaccuo and the residue was taken up in 100 mL of EtOAc and washed with saturated NaHCO₃. The organic layer was dried over MgSO₄, loaded onto silica, and purified via silica chromatography using an EtOAc/Hexanes 0-100% gradient, to afford 0.87 g (85% yield) of N-{3-[5-(2-chloro-4-pyrimidinyl)-1,3-thiazol-4-yl]phenyl}-2,5-difluorobenzamide as a pale brown solid. ¹H-NMR (400 MHz, DMSO-D₆) δ 10.6 (s, 1H), 9.4 (s, 1H), 8.6 (d, J=5.3 Hz, 1H), 8.0 (s, 1H), 7.8 (dd, J=8.2 and 1.6 Hz, 1H), 7.5 (m, 1H), 7.4 (m, 3H), 7.3 (d, J=7.9 Hz, 1H), and 7.3 (d, J=5.3 Hz, 1H); ESIMS m/z 429 (M+H).

### Step B: N-[3-(5-{2-[(3-{[2-(Dimethylamino)ethyl]oxy}phenyl)amino]-4-pyrimidinyl}-1,3-thiazol-4-yl)phenyl]-2,5-difluorobenzamide

Title compound of Example 96 was synthesized using standard chloride displacement conditions in a procedure analogous to Example 1, Step D, using N-{3-[5-(2-chloro-4-pyrimidinyl)-1,3-thiazol-4-yl]phenyl}-2,5-difluor obenzamide (0.073 g, 0.17 mmol) and {2-[(3-aminophenyl)oxy]ethyl}dimethylamine hydrochloride (0.040 g, 0.19 mmol) in the microwave and purified by silica chromatography to give 0.021 g (22% yield) of the title compound of Example 96. ¹H-NMR (400 MHz, DMSO-D₆) δ 8.5 (d, J=15.9 Hz, 1H), 8.2 (d, J=5.1 Hz, 1H), 7.9 (m, 3H), 7.5 (m, 2H), 7.4 (s, 1 H), 7.3 (s, 2H), 7.2 (m, 3H), 7.1 (d, J=9.3 Hz, 1H), 6.6 (m, 2H), 4.2 (t, J=5.8 Hz, 2H), 2.8 (t, J=5.6 Hz, 2H), and 2.4 (s, 6H); ESIMS m/z 573 (M+H).

### Example 97

### N-[3-(5-{2-[(3-{[2-(Diethylamino)ethyl]oxy}phenyl)amino]-4-pyrimidinyl}-1,3-thiazol-4-yl)phenyl]-2,5-difluorobenzamide

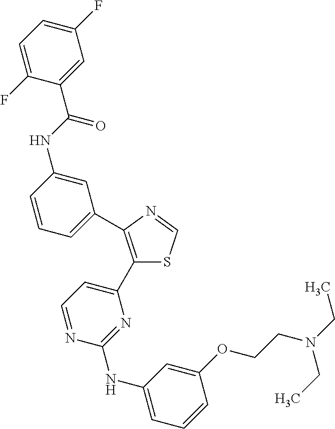

Title compound of Example 97 was synthesized using standard chloride displacement conditions in a procedure analogous to Example 1, Step D, using N-{3-[5-(2-chloro-4-pyrimidinyl)-1,3-thiazol-4-yl]phenyl}-2,5-difluorobenzamide (0.073 g, 0.17 mmol), prepared by a procedure analogous to Example 96, Step A, and 3-{[2-(diethylamino)ethyl]oxy}phenyl amine (0.038 g, 0.19 mmol) in the microwave and purified by silica chromatography to give 0.024 g, (23% yield) of the title compound of Example 97. ¹H-NMR (400 MHz, DMSO-D₆) δ 10.6 (s, 1H), 9.7 (s, 1H), 9.3 (m, 1 H), 8.4 (d, J=5.1 Hz, 1H), 8.0 (s, 1H), 7.8 (d, J=8.1 Hz, 1H), 7.5 (s, 2H), 7.4 (m, 3H), 7.3 (dd, J=19.5 and 8.0 Hz, 2H), 7.1 (t, J=8.1 Hz, 1H), 6.6 (d, J=5.1 Hz, 1H), 6.5 (d, J=8.1 Hz, 1H), 4.0 (t, J=6.2 Hz, 2H), 2.8 (t, J=6.1 Hz, 2H), 2.5 (q, J=7.1 Hz, 4H), and 1.0 (s, 6H); ESIMS m/z 601 (M+H).

### Example 98

### 2,5-Difluoro-N-[3-(5-{2-[(3-oxo-3,4-dihydro-2H-1,4-benzoxaziN-7-yl)amino]-4-pyrimidinyl}-1,3-thiazol-4-yl)phenyl]benzamide hydrochloride

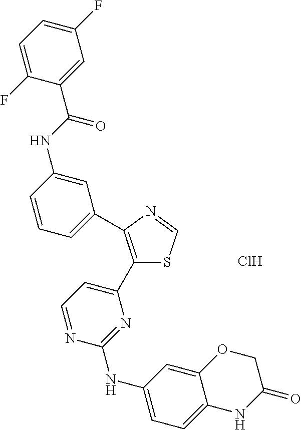

Title compound of Example 98 was synthesized using standard chloride displacement conditions in a procedure analogous to Example 1, Step D, using N-{3-[5-(2-chloro-4-pyrimidinyl)-1,3-thiazol-4-yl]phenyl}-2,5-difluorobenzamide (0.073 g, 0.17 mmol), prepared by a procedure analogous to Example 96, Step A, and 7-amino-2H-1,4-benzoxazin-3(4H)-one (0.039 g, 0.19 mmol) in the microwave and solid precipitate was filtered and rinsed with ether to give 0.048 g, (51% yield) of the title compound of Example 98. ¹H-NMR (400 MHz, DMSO-D₆) δ 10.4 (s, 1H), 9.7 (m, 1H), 9.4 (s, 1H), 9.3 (m, 1H), 8.0 (s, 1H), 7.5 (s, 2H), 7.5 (m, 2H), 7.3 (m, 2H), 6.8 (d, J=8.6 Hz, 1H), 6.6 (d, J=8.1 Hz, 1H), 6.3 (d, J=10.8 Hz, 2H), and 4.4 (s, 2H); ESIMS m/z 557 (M+H).

### Example 99

### 2,5-Difluoro-N-[3-(5-{2-[(3-oxo-3,4-dihydro-2H-1,4-benzoxaziN-6-yl)amino]-4-pyrimidinyl}-1,3-thiazol-4-yl)phenyl]benzamide hydrochloride

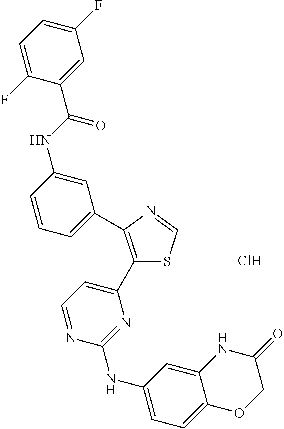

Title compound of Example 99 was synthesized using standard chloride displacement conditions in a procedure analogous to Example 1, Step D, using N-{3-[5-(2-chloro-4-pyrimidinyl)-1,3-thiazol-4-yl]phenyl}-2,5-difluorobenzamide (0.073 g, 0.17 mmol), prepared by a procedure analogous to Example 96, Step A, and 6-amino-2H-1,4-benzoxazin-3(4H)-one (0.039 g, 0.19 mmol) in the microwave. Solid precipitate was filtered and rinsed with ether to give 0.051 g, (54% yield) of the title compound of Example 99. ¹H-NMR (400 MHz, DMSO-D₆) δ 10.7 (s, 1H), 9.7 (s, 1 H), 9.3 (s, 1H), 8.3 (d, J=5.3 Hz, 1H), 7.9 (s, 1H), 7.8 (d, J=7.0 Hz, 1H), 7.5 (m, 1H), 7.4 (m, 3H), 7.3 (d, J=7.3 Hz, 1H), 7.2 (d, J=8.6 Hz, 1H), 6.8 (d, J=8.4 Hz, 1H), 6.6 (m, 1H), 6.5 (d, J=5.1 Hz, 1H), and 4.5 (s, 2H); ESIMS m/z 557 (M+H).

### Example 100

### N-(3-{5-[2-({3-[2-(Aminosulfonyl)ethyl]phenyl}amino)-4-pyrimidinyl]-1,3-thiazol-4-yl}phenyl)-2,5-difluorobenzamide trifluoroacetate

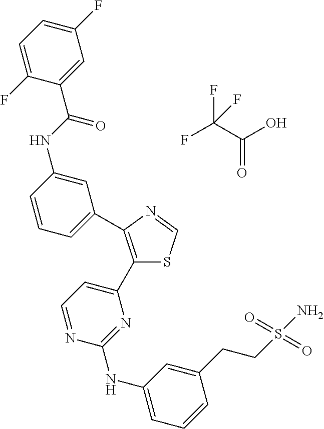

Title compound of Example 100 was synthesized using standard chloride displacement conditions in a procedure analogous to Example 1, Step D, using N-{3-[5-(2-chloro-4-pyrimidinyl)-1,3-thiazol-4-yl]phenyl}-2,5-difluorobenzamide (0.073 g, 0.17 mmol), prepared by a procedure analogous to Example 96, Step A, and 2-(3-aminophenyl)ethanesulfonamide (0.038 g, 0.19 mmol), prepared by a procedure analogous to Example 8, Step A, in the microwave and purified by HPLC to give 0.028 g, (27% yield) of the title compound of Example 100. ¹H-NMR (400 MHz, DMSO-D₆) δ 10.6 (s, 1H), 9.7 (s, 1H), 9.3 (s, 1H), 8.3 (d, J=5.1 Hz, 1H), 8.0 (s, 1H), 7.8 (d, J=7.5 Hz, 1H), 7.7 (s, 1H), 7.5 (m, 2H), 7.4 (m, 3H), 7.3 (d, J=7.7 Hz, 1H), 7.2 (t, J=7.8 Hz, 1H), 6.9 (m, 3H), 6.6 (d, J=5.1 Hz, 1H), 3.2 (s, 2H), and 3.0 (s, 2H); ESIMS m/z 593 (M+H).

### Example 101

### 2,6-Difluoro-N-[3-(5-{2-[(3-fluorophenyl)amino]-4-pyrimidinyl}-2-methyl-1,3-thiazol-4-yl)phenyl]benzamide

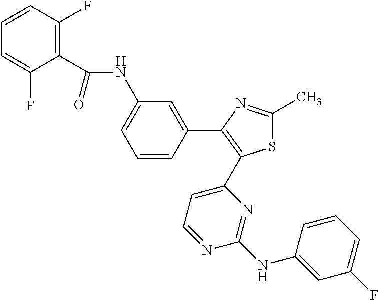

### Step A: N-{3-[2-Bromo-5-(2-chloro-4-pyrimidinyl)-1,3-thiazol-4-yl]phenyl}-2,6-difluorobenzamide

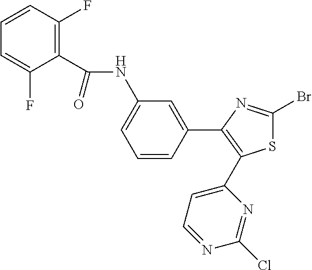

To prepare the title compound of Step A, N-{3-[2-amino-5-(2-chloro-4-pyrimidinyl)-1,3-thiazol-4-yl]phenyl}-2,6-difluorobenzamide (0.2 g, 0.45 mmol), prepared by a procedure analogous to Example 2, Step A, was dissolved in 15 mL acetonitrile and the resulting mixture was cooled in a 0° C. ice bath. Tert-butyl nitrite (0.24 mL, 0.67 mmol) was then added drop-wise over 1 minute. The ice bath was removed and Cu(II)Br was added in one portion. The reaction was stirred 15 min, H₂O was added, and the subsequent mixture was extracted with DCM. After further washing of the DCM with brine, the resulting organic layers were concentrated onto silica gel. Chromatography with EtOAc and hexanes yielded 200 mg (86%) of the title compound of Step A. ¹H-NMR (300 MHz, DMSO-d₆) δ 10.95 (s, 1H), 8.65 (d, J=5.6 Hz, 1H), 7.95 (s, 1H), 7.80 (d, J=7.8 Hz, 1H), 7.60 (m, 1H), 7.49 (t, J=8.1 Hz, 1H), 7.36 (d, J=7.9 Hz, 1H), and 7.30-7.21 (m, 3H).

### Step B: N-{3-[5-(2-Chloro-4-pyrimidinyl)-2-methyl-1,3-thiazol-4-yl]phenyl}-2,6-difluorobenzamide

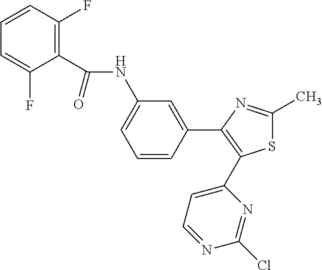

To prepare the title compound of Step B, N-{3-[2-bromo-5-(2-chloro-4-pyrimidinyl)-1,3-thiazol-4-yl]phenyl}-2,6-difluorobenzamide (0.484 g, 0.95 mmol) was dissolved in THF (10 mL). Palladium tetrakis triphenylphosphine (0.095 g, 0.095 mmol) was added, followed by the drop-wise addition of 2 M dimethylzinc in toluene (0.5 mL, 1.0 mmol). The reaction was stirred at rt for 30 min and H₂O was added to quench the reaction. This was followed by extraction of the reaction with EtOAc. The resulting organics were combined and concentrated onto silica gel. Chromatography using EtOAc and hexanes yielded pure fractions that, after concentration, gave 300 mg (70%) of the title compound of Step B. ¹H-NMR (300 MHz, DMSO-d₆) δ 10.91 (s, 1 H), 8.58 (d, J=5.3 Hz, 1H), 7.93 (s, 1H), 7.77 (d, J=8.2 Hz, 1H), 7.65-7.52 (m, 2H), 7.45 (t, J=7.9 Hz, 1H), 7.32 (d, J=7.8 Hz, 1H), 7.29-7.19 (m, 3H), and 2.74 (s, 3H).

### Step C: 2,6-Difluoro-N-[3-(5-{2-[(3-fluorophenyl)amino]-4-pyrimidinyl}-2-methyl-1,3-thiazol-4-yl)phenyl]benzamide

The title compound of Example 101 was prepared from N-{3-[5-(2-chloro-4-pyrimidinyl)-2-methyl-1,3-thiazol-4-yl]phenyl}-2,6-difluorobenzamide (0.08 g, 0.19 mmol) and 3-fluorophenyl amine (0.02 g, 0.17 mmol) by a procedure analogous to Example 1, Step D. Yield: 60 mg (64%). ¹H-NMR (300 MHz, DMSO-d₆) δ 10.89 (s, 1H), 9.96 (s, 1H), 8.36 (d, J=5.4 Hz, 1H), 7.94 (s, 1H), 7.79-7.71 (m, 2H), 7.58 (m, 1H), 7.49-7.40 (m, 2H), 7.33-7.20 (m, 4H), 6.75 (t, J=8.6 Hz, 1H), 6.61 (d, J=5.0 Hz, 1H), and 2.74 (s, 3H); HRMS C₂₇H₁₉N₆OS (M+H)⁺ calcd 518.1257. found 518.1255.

### Example 102

### N-[3-(5-{2-[(3-{[2-(Dimethylamino)ethyl]oxy}phenyl)amino]-4-pyrimidinyl}-2-methyl-1,3-thiazol-4-yl)phenyl]-2,6-difluorobenzamide

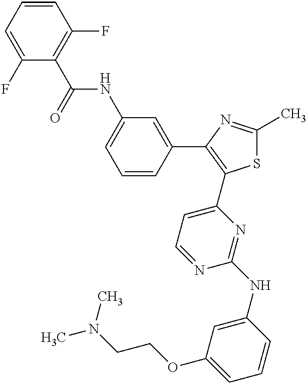

The title compound of Example 102 was prepared from N-{3-[5-(2-chloro-4-pyrimidinyl)-2-methyl-1,3-thiazol-4-yl]phenyl}-2,6-difluorobenzamide (0.08 g, 0.18 mmol) prepared by a procedure analogous to Example 100, Step D, and {2-[(3-aminophenyl)oxy]ethyl}dimethylamine hydrochloride (0.04 g, 0.18 mmol), by a procedure analogous to Example 1, Step D. Yield 55 mg (51%). ¹H-NMR (300 MHz, DMSO-d₆) δ 10.93 (s, 1H), 9.76 (s, 1H), 8.33 (d, J=5.5 Hz, 1H), 7.96 (s, 1H), 7.74 (d, J=8.2 Hz, 1H), 7.65-7.53 (m, 2H), 7.45 (t, J=7.8 Hz, 1H), 7.33-7.14 (m, 5H), 6.62-6.51 (m, 2H), 4.24 (m, 2H), 3.23 (br, 2H), 2.73 (s, 3H), and 2.65 (s, 6H); HRMS C₃₁H₂₉N₆O₂F₂S (M+H)⁺ calcd 587.2041. found 587.2036.

### Example 103

### N-[3-(5-{2-[(3-Chloro-4-{[2-(dimethylamino)ethyl]oxy}phenyl)amino]-4-pyrimidinyl}-2-methyl-1,3-thiazol-4-yl)phenyl]-2,6-difluorobenzamide

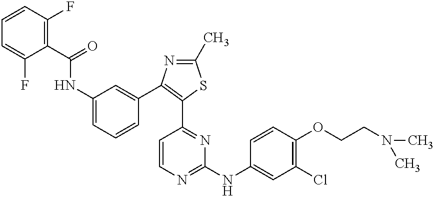

### Step A: 4-[2-Bromo-4-(3-nitrophenyl)-1,3-thiazol-5-yl]-2-(methylthio)pyrimidine

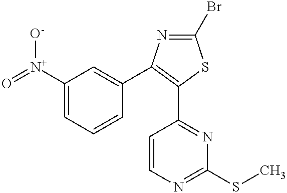

t-Butyl nitrite was added to a stirring mixture of 5-[2-(methylthio)-4-pyrimidinyl]-4-(3-nitrophenyl)-1,3-thiazol-2-amine (1.13 g, 3.27 mmol), prepared by a procedure analogous to Example 87, Step B, in acetonitrile (35 mL) at 0° C. Copper (II) bromide (3.45 mmol) was added and the resulting mixture was stirred at 0° C. for 0.5 h. The reaction was partitioned between EtOAc and H₂O (2:1, 300 mL). The aqueous phase was re-extracted with EtOAc (100 mL) and the combined organics were washed with brine and filtered through Whatman 1PS paper. The filtrate was concentrated in vacuo to a crude solid residue (1.14 g, 85%). ¹H-NMR (400 mHz, CDCl₃-d) δ 8.48 (s, 1H), 8.34 (brs, 1H), 8.31 (d, 1H, J=8.2 Hz), 7.89 (d, 1H, J=7.7 Hz), 7.63 (t, 1H, J=8.0 Hz), 6.72 (d, 1H, J=5.1 Hz), and 2.53 (s, 3H) MS (ESI) 410 [M+H].

### Step B: 4-[2-Bromo-4-(3-nitrophenyl)-1,3-thiazol-5-yl]-2-(methylsulfonyl)pyrimidine

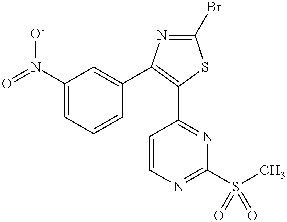

3-Chloroperoxybenzoic acid (8.83 mmol) was added to a stirring mixture of 4-[2-bromo-4-(3-nitrophenyl)-1,3-thiazol-5-yl]-2-(methylthio)pyrimidine (1.0 g, 2.44 mmol) in DCM, stirring at 0° C. The reaction was stirred for 0.5 h at 0° C. and then warmed to rt. The reaction was diluted further with DCM and washed with 10% sodium thiosulfate and saturated NaHCO₃ solutions. The organic phase was filtered through Whatman PS1 paper and concentrated to crude residue (0.85 g, 79%). ¹H-NMR (400 mHz, CDCl₃-d): δ 8.73 (d, 1H, J=5.3 Hz), 8.47 (brs, 1H), 8.36 (d, 1H, J=8.1 Hz), 7.90 (d, 1H, J=7.7 Hz), 7.68 (t, 1H, J=8.0 Hz), 7.26 (d, 1H, J=5.3 Hz), and 3.33 (s, 3H).

### Step C: 4-[2-Methyl-4-(3-nitrophenyl)-1,3-thiazol-5-yl]-2-(methylsulfonyl)pyrimidine

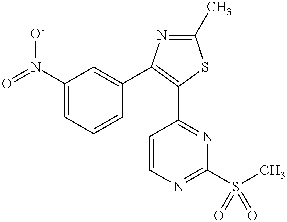

To a 50 mL round bottom flask purged with N₂ was added 4-[2-bromo-4-(3-nitrophenyl)-1,3-thiazol-5-yl]-2-(methylsulfonyl)pyrimidine (0.59 g, 1.34 mmol), dichloro[1,1′-bis(diphenylphoshino)ferrocine]palladium (II) DCM adduct (0.21 mmol), copper (I) iodide (0.53 mmol) and THF (3 mL). (Methylzinc)chloride (2 M in THF, 3 mmol) was added via syringe and the resulting mixture was refluxed for 0.5 h. The reaction was diluted with DCM and absorbed directly to silica gel. Purification by chromatography (25-100% EtOAc to hexanes) gave the desired product (0.56 g; 77%). ¹H-NMR (d, 1H, J=8.2 Hz), 7.89 (d, 1H, J=7.7 Hz), 7.65 (t, 1H, J=8.0 Hz), 7.26 (s, 1H), 3.29 (s, 3H), and 2.81 (s, 3H); MS (ESI) 377 [M+H] 375 [M−H].

### Step D: 4-[2-Methyl-4-(3-nitrophenyl)-1,3-thiazol-5-yl]-2(1H)-pyrimidinone

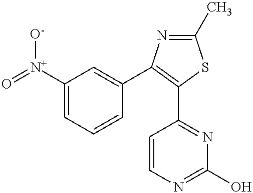

4-[2-Methyl-4-(3-nitrophenyl)-1,3-thiazol-5-yl]-2-(methylsulfonyppyrimidine (0.66 g, 1.49 mmol, from multiple batches prepared according to the foregoing procedure) was heated to 90° C. in concentrated HCl (10 mL) for 1 h. The reaction was diluted with MeOH and concentrated in vacuo to a semi-solid residue. The residue was co-evaporated with toluene (2×100 mL) to dryness. The residue was triturated with DCM and the solvent was removed by decanting and the remaining solid concentrated in vacuo to a solid residue (0.54 g). MS (ESI) 315 {M+H].

### Step E: 2-Chloro-4-[2-methyl-4-(3-nitrophenyl)-1,3-thiazol-5-yl]pyrimidine

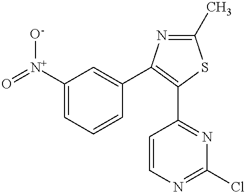

Phosphorus oxychloride (10 mL) and 4-[2-methyl-4-(3-nitrophenyl)-1,3-thiazol-5-yl]-2(1H)-pyrimidinone (0.53 g, 1.69 mmol) was heated at 90° C. for 3.5 h. The reaction was poured into iced H₂O and extracted with EtOAc. The organic phase was washed with brine, filtered through Whatman 1PS paper and concentrated in vacuo to a crude residue. ¹H-NMR (400 mHz, CDCl₃-d): δ 8.50 (brs 1H), 8.41 (d, 1H J=4.4 Hz), 8.32 (d, 1H, J=8.2 Hz), 7.88 (d, 1H, J=7.5 Hz), 7.64 (t, 1H, J=8.0 Hz), 7.00 (d, 1H, J=5.1 Hz), and 2.80 (s, 3H); MS (ESI) 333 [M+H].

### Step F: 3-[5-(2-Chloro-4-pyrimidinyl)-2-methyl-1,3-thiazol-4-yl]phenyl amine

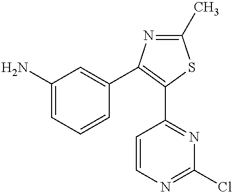

Hydrogenation on the Fisher-Porter apparatus of 2-chloro-4-[2-methyl-4-(3-nitrophenyl)-1,3-thiazol-5-yl]pyrimidine (1.3 g, 4.21 mmol, prepared from multiple batches according to the foregoing procedure) in EtOH (50 mL) with 5% sulfided platinum on carbon (0.775 g, 0.199 mmol) at 50 psi for 16 h gave the desired product of Step F (1.28 g, 100%). ¹H-NMR (400 mHz, CDCl₃-d): δ 8.32 (d, 1H, J=5.5 Hz) 7.23 (d, 1H, J=7.7 Hz), 7.08 (d, 1H, J=5.5 Hz), 6.95 (s, 1H), 6.91 (d, 1H, J=7.7 Hz), 6.87 (d, 1H, J=7.87 Hz), and 2.77 (s, 3H); MS (ESI) 303 [M+H].

### Step G: N-{3-[5-(2-Chloro-4-pyrimidinyl)-2-methyl-1,3-thiazol-4-yl]phenyl}-2,6-difluorobenzamide

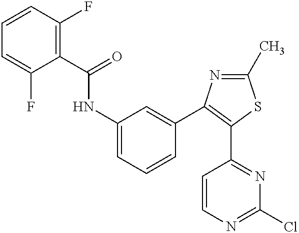

Method 1: In a procedure similar to Example 1, Step A, 2,6-difluorobenzoyl chloride (0.7 g, 3.96 mmol) was added to 3-[5-(2-chloro-4-pyrimidinyl)-2-methyl-1,3-thiazol-4-yl]phenyl amine (1.2 g, 3.96 mmol) in DCM (50 mL) at 0° C. The reaction was stirred at rt for 0.5 h. MP-carbonate was added to the reaction and the reaction was stirred for 0.5 h. The reaction was diluted with MeOH, filtered, and concentrated in vacuo to a crude residue. Trituration of the crude solid with MeOH gave a tacky residue that was dissolved in DCM and evaporated to give the desired product of Step G (0.96 g, 55%). ¹H-NMR (400 mHz, CDCl₃-d): δ 8.36 (d, 1H, J=5.3 Hz) 7.82-7.77 (m, 2H), 7.71 (s, 1H), 7.48-7.4 (m, 2H), 7.32 (d, 1H, J=7.5 Hz), 7.13 (d, 1H, J=7.3 Hz), 7.00 (t, 2H, J=8.3 Hz), and 2.80 (s, 3H); MS (ESI) 443 [M+H].

Method 2: NBS (2.35 g, 13.2 mmol) was added to a stirring suspension of N-{3-[(2-chloro-4-pyrimidinyl)acetyl]phenyl}-2,6-difluorobenzamide, prepared by a procedure analogous to Example 2, Step A, (5.19 g; 13.38 mmol) in DCM (250 mL). The resulting clear, homogeneous reaction was stirred for 0.5 h at rt. No evidence of the original starting material was found by LC/MS and the reaction was concentrated in vacuo to a crude residue. This residue was taken up in DMF (100 mL) and thioacetamide (1.04 g; 13.84 mmol) in DMF (10 mL) was added. The reaction was heated at 80° C. and the LC/MS at 0.5 h showed the absence of the starting intermediate. The reaction was poured into water and was extracted with EtOAc and concentrated in vacuo to a crude residue. Purification by column chromatography (10 to 100% EtOAc: DCM) provided a 3.85 g (66%) of the title compound of Step G. ¹H-NMR (400 mHz, CDCl₃-d): δ 8.36 (d, 1H, J=5.3 Hz) 7.82-7.77 (m, 2H), 7.71 (s, 1H), 7.48-7.4 (m, 2H), 7.32 (d, 1H, J=7.5 Hz), 7.13 (d, 1H, J=7.3 Hz), 7.00 (t, 2H, J=8.3 Hz), and 2.80 (s, 3H); MS (ESI) 443 [M+H].

### Step H: N-[3-(5-{2-[(3-Chloro-4-{[2-(dimethylamino)ethyl]oxy}phenyl)amino]-4-pyrimidinyl}-2-methyl-1,3-thiazol-4-yl)phenyl]-2,6-difluorobenzamide

To N-{3-[5-(2-chloro-4-pyrimidinyl)-2-methyl-1,3-thiazol-4-yl]phenyl}-2,6-difluorobenzamide (0.08 g; 0.18 mmol) in i-PrOH (2 mL) was added {2-[(4-amino-2-chlorophenyl)oxy]ethyl}dimethylamine hydrochloride (0.047 g; 0.19 mmol) with 3 drops of concentrated hydrochloric acid. The reaction was microwaved at 185° C. for 10 min (3×). The reaction was diluted with DCM and washed with saturated NaHCO₃ solution. The organics were filtered through Whatman 1 PS paper and concentrated in vacuo to a crude residue. Purification by column chromatography (0 to 100%) eluting with DCM:(DCM:MeOH:NH₄OH)/40:9:1 did not yield a pure product. Purification by RP HPLC provided the desired product (0.028 g, 25%). ¹H-NMR (400 mHz, CDCl₃-d) δ 8.18 (d, 1H, J=5.1 Hz), 7.82 (d, 1H, J=7.9 Hz), 7.77 (brs, 2H), 7.67 (brs, 1H), 7.45-7.40 (m, 2H), 7.38-7.03 (m, 2H), 7.01 (brs, 2H), 6.98 (d, 1 H, J=8.1 Hz), 6.91 (d, 1H, J=8.8 Hz), 6.61 (d, 1H, J=5.1 Hz), 4.12 (t, 2H, J=5.8 Hz), 2.78 (d, 2H, J=5.9 Hz), 2.76 (s, 3H), and 2.36 (s, 6H); MS (ESI) 621 [M].

### Example 104

### 2,6-Difluoro-N-{3-[2-methyl-5-(2-{[3-(1-pyrrolidinylmethyl)phenyl]amino}-4-pyrimidinyl)-1,3-thiazol-4-yl]phenyl}benzamide

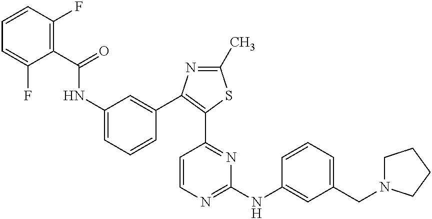

A reaction containing N-{3-[5-(2-chloro-4-pyrimidinyl)-2-methyl-1,3-thiazol-4-yl]phenyl}-2,6-difluorobenzamide (0.1 g; 0.23 mmol), prepared by a procedure analogous to Example 103, Step G, 3-(1-pyrrolidinylmethyl)phenyl amine in i-PrOH with 3 drops of concentrated HCl was heated with stirring at 90° C. for 16 h. The reaction was concentrated in vacuo to a crude residue. Purification using RP HPLC gave the desired product (0.033 g, 25%). ¹H-NMR (400 mHz, CDCl₃-d) δ 8.20 (d, 1 H, J=5.3 Hz), 7.87 (d, 1H, J=8.2 Hz), 7.76 (brs, 1H), 7.72 (brs, 1H), 7.62 (d, 1H), 7.55 (brs, 1H), 7.45-7.4 (m, 2H), 7.36 (brs, 1H), 7.34 (s, 1H), 7.33 (t, 1H, J=7.9), 7.18 (s, 1H), 7.06 (d, 1H, J=7.5 Hz) 7.01 (t, 2H, J=8.2 Hz), 6.62 (d, 1H, J=5.1 Hz), 3.67 (brs, 2H), 2.77 (s, 3H), 2.61 (brs, 4H), and 1.83 (brs, 4H); MS (ESI) 583 [M+H] 581 [M−H].

### Example 105

### N-[3-(5-{2-[(4-{[2-(Dimethylamino)ethyl]oxy}-3-fluorophenyl)amino]-4-pyrimidinyl}-2-methyl-1,3-thiazol-4-yl)phenyl]-2,6-difluorobenzamide

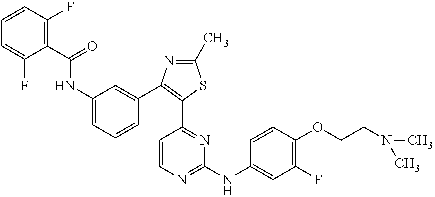

### Step A: {2-[(2-Fluoro-4-nitrophenyl)oxy]ethyl}dimethylamine

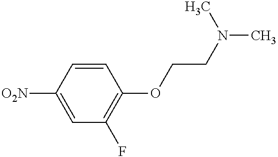

A solution of 1,2-difluoro-4-nitrobenzene (2.0 mL, 18.1 mmol), 2-(dimethylamino)ethanol (2.2 mL, 22.0 mmol) and cesium carbonate (29.4 g, 90.5 mmol) in DMF (10 mL) was heated to 90° C. for 24 hours. The reaction mixture was diluted with DCM and washed with H₂O. The organics were dried over MgSO₄ and concentrated onto silica gel. Purification by column chromatography (20-100% 1:9:90 NH₄OH:MeOH:DCM in DCM) provided 2.9 g (71%) as a yellow oil. MS (ESI): 229.08 [M+H]⁺.

### Step B: 4-{[2-(Dimethylamino)ethyl]oxy}-3-fluorophenyl amine

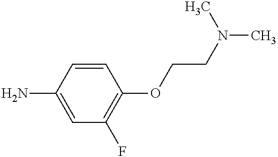

A solution of {2-[(2-fluoro-4-nitrophenyl)oxy]ethyl}dimethylamine (2.9 g, 12.7 mmol) and sulfided platinum on carbon (500 mg) in EtOH (50 mL) was stirred under 60 psi of H₂ on a Fischer-Porter hydrogenator for two hours. The reaction mixture was diluted with EtOAc and filtered through celite (yellow oil, 2.6 g, 13.1 mmol). The filtrate was concentrated, diluted with MeOH (10 mL) and ether (50 mL). The phenyl amine was then treated with 1 M HCl in ether (13.1 mmol). The HCl salt was filtered and rinsed with ether providing 2.9 g (100%) as a beige solid. ¹H-NMR (400 MHz, DMSO-d6): δ 7.08 (t, 1H, J=9.1 Hz), 6.76 (d, 1H, J=12.3 Hz), 6.65 (d, 1H, J=8.2 Hz), 4.28 (m, 2H), 3.44 (t, 2H, J=4.9 Hz), and 2.81 (s, 6H).

### Step C: N-[3-(5-{2-[(4-{[2-(Dimethylamino)ethyl]oxy}-3-fluorophenyl)amino]-4-pyrimidinyl}-2-methyl-1,3-thiazol-4-yl)phenyl]-2,6-difluorobenzamide

The preparation was carried out in a manner analogous to Example 1, Step D, using N-{3-[5-(2-chloro-4-pyrimidinyl)-2-methyl-1,3-thiazol-4-yl]phenyl}-2,6-difluorobenzamide (0.1 g, 0.23 mmol), prepared by a procedure analogous to Example 103, Step G, with {2-[(4-amino-2-fluorophenyl)oxy]ethyl}dimethylamine hydrochloride (0.052 g, 0.22 mmol). Purification using RP HPLC gave the desired product (0.027 g, 19%). ¹H-NMR (400 mHz, CDCl₃-d) δ 8.18 (d, 1H, J=5.3 Hz), 7.82 (d, 1H, J=8.2 Hz), 7.77 (brs, 1H), 7.67 (brs, 1H), 7.61 (dd, 1H, J=13.5 Hz and J=2.5 Hz), 7.41 (t, 2H), 7.33 (d, 1H), 7.10 (d, 1H, J=8.8 Hz), 7.06 (s, 1H), 7.02-6.9 (m, 3H), 6.62 (d, 1H, J=5.3 Hz), 4.14 (t, 2H, J=5.6 Hz), 2.79 (brs, 2H), 2.75 (s, 3H), and 2.38 (s, 6H); MS (ESI) 603 [M−H].

### Example 106

### Phenylmethyl 4-{[4-({4-[4-(3-{[2,6-difluorophenyl)carbonyl]amino}phenyl)-2-methyl-1,3-thiazol-5-yl]-2-pyrimidinyl}amino)-2-fluorophenyl]oxy}-1-piperidinecarboxylate

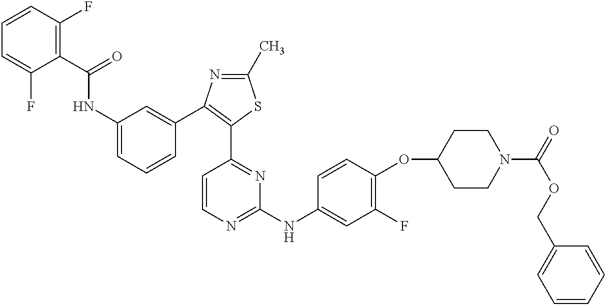

The general procedure of Example 1, Step D, was used for the reaction of N-{3-[5-(2-chloro-4-pyrimidinyl)-2-methyl-1,3-thiazol-4-yl]phenyl}-2,6-difluorobenzamide (1.5 g, 3.39 mmol), prepared by a procedure analogous to Example 103, Step G, and phenylmethyl-4-[(4-amino-2-fluorophenyl)oxy]-1-piperidinecarboxylate (1.25 g, 3.63 mmol) at 90° C. overnight to give a crude product (1.18 g, 46%). Purification by RP HPLC gave the desired product. ¹H-NMR (400 mHz, CDCl₃-d) δ 8.18 (d, 1H, J=5.1 Hz), 7.80 (d, 1H, J=8.1 Hz), 7.80 (brs, 1H), 7.67 (brs, 1H), 7.68-7.62 (m, 2H), 7.42 (t, 2H), 7.35 (m, 5H), 7.09 (m, 2H), 7.01-6.93 (m, 3H), 6.64 (d, 1H, J=5.3 Hz), 5.13 (s, 2H), 4.37-4.33 (m, 1H), 3.82-3.75 (m, 2H), 3.45-3.39 (m, 2H), 2.76 (s, 3H), 1.89 (brs, 2H), and 1.81 (brs, 2H); MS (ESI) 751 [M−H].

### Example 107

### 2,6-DUDuoro-N-{3-[5-(2-{[3-fluoro-4-(4-piperidinyloxy)phenyl]amino}-4-pyrimidinyl)-2-methyl-1,3-thiazol-4-yl]phenyl}benzamide

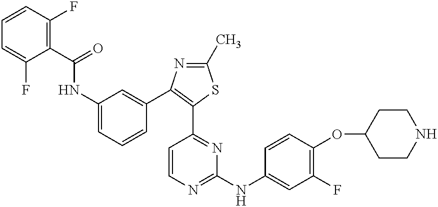

Transfer hydrogenation of phenylmethyl 4-{[4-({4-[4-(3-{[(2,6-difluorophenyl)carbonyl]amino}phenyl)-2-methyl-1,3-thiazol-5-yl]-2-pyrimidinyl}amino)-2-fluorophenyl]oxy}-1-piperidinecarboxylate (1.0 g, 1.33 mmol), prepared according to a procedure analogous to Example 106, with 10% palladium on carbon (0.15 g, 0.14 mmol) in EtOH (10 mL) and cyclohexene (5 mL) gave a crude residue after filtration with Celite. Pure product (0.030 g) was obtained by RP HPLC. ¹H-NMR (400 mHz, CDCl₃-d) δ 8.20 (d, 1H, J=5.3 Hz), 7.83 (d, 1H, J=8.2 Hz), 7.79 (brs, 1H), 7.67 (dd, 2H, J=2.7 Hz and J=13.3 Hz), 7.45-7.42 (m, 2H), 7.35 (d, 1H, J=7.7), 7.12-6.95 (m, 2H), 6.64 (d, 1H, J=5.1 Hz), 4.29-4.21 (m, 1 H), 3.20-3.13 (m, 2H), 2.77 (s, 3H), 2.72-2.66 (m, 2H), 2.07-1.98 (m, 2H), and 1.74-1.66 (m, 2H); MS (ESI) 617 [M+H].

### Example 108

### 2,6-Difluoro-N-(3-{5-[2-({3-fluoro-4-[(1-methyl-4-piperidinyl)oxy]phenyl}amino)-4-pyrimidinyl]-2-methyl-1,3-thiazol-4-yl}phenyl)benzamide

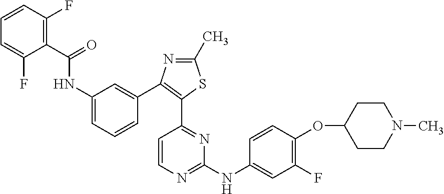

To a stirring solution of 2,6-difluoro-N-{3-[5-(2-{[3-fluoro-4-(4-piperidinyloxy)phenyl]amino}-4-pyrimidinyl)-2-methyl-1,3-thiazol-4-yl]phenyl}benzamide (0.1 g, 0.16 mmol), prepared by a procedure analogous to Example 105, in DCM (3 mL) and MeOH (1.5 mL), was added formaldehyde (0.015 g, 0.18 mmol), HOAc (0.25 mmol) and sodium triacetoxyborohydride (0.05 g, 0.24 mmol). The reaction was stirred at rt for 1 h. The reaction was quenched with saturated NaHCO₃ solution and water and extracted with EtOAc (15 mL). The EtOAc was filtered through Whatman 1PS paper and concentrated to a crude residue. Purification by RP HPLC gave the desired product (0.025 g, 25%). ¹H-NMR (400 mHz, CDCl₃-d) δ 8.20 (d, 1H, J=5.1 Hz), 7.80 (m, 2H), 7.68-7.64 (m, 2H), 7.44-7.38 (m, 2H), 7.45-7.42 (m, 2H), 7.33 (d, 1H, J=7.5 Hz), 7.10 (s, 1H), 7.08 (brs, 1H), 7.04-6.93 (m, 3H), 6.64 (d, 1H, J=5.1 Hz), 4.30 (br, 1H), 2.90 (brs, 2H), 2.76 (s, 3H), 2.44 (brs, 2H), 2.00 (brs, 2H), and 1.56 (brs, 5H); MS (ESI) 631 [M+H] and 629 [M−H].

### Example 109

### N-(3-{5-[2-({4-[(1-Acetyl-4-piperidinyl)oxy]-3-fluorophenyl}amino)-4-pyrimidinyl]-2-methyl-1,3-thiazol-4-yl}phenyl)-2,6-difluorobenzamide

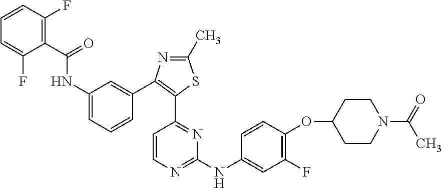

Acetic anhydride (0.025 g, 0.24 mmol) was added to a chilled solution of 2,6-difluoro-N-{3-[5-(2-{[3-fluoro-4-(4-piperidinyloxy)phenyl]amino}-4-pyrimidinyl)-2-methyl-1,3-thiazol-4-yl]phenyl}benzamide (0.1 g, 0.16 mmol), prepared by a procedure analogous to Example 107, in DCM (2 mL) and stirred for 0.5 h. The reaction was diluted with DCM and washed with saturated NaHCO₃ solution. The aqueous phase was further extracted with EtOAc and the combined organics washed with H₂O. The organic phase was filtered through Whatman 1PS paper and concentrated in vacuo to a crude residue. Purification by RP HPLC gave the desired product (0.036 g, 33%). ¹H-NMR (400 mHz, CDCl₃-d) δ 8.17 (d, 1H, J=5.3 Hz), 7.84 (s, 1H), 7.79 (d, 1H, J=8.1 Hz), 7.71 (s, 1H), 7.67 (dd, 2H, J=13.3 Hz and J=2.5 Hz), 7.46-7.42 (m, 2H), 7.35 (d, 1H, J=7.7 Hz), 7.12 (d, 1H, J=8.8 Hz), 7.03-6.96 (m, 3H), 6.68 (d, 1H, J=5.3 Hz), 4.47-4.43 (m, 1H), 3.82-3.78 (m, 2H), 3.65-3.35 (m, 2H), 2.78 (s, 3H), 2.12 (s, 3H), and 2.0-1.8 (brm, 4H); MS (ESI) 659 [M+H] and 657 [M−H].

### Example 110

### N-[3-(5-{2-[(3-Chloro-4-{[2-(1-pyrrolidinyl)ethyl]oxy}phenyl)amino]-4-pyrimidinyl}-2-methyl-1,3-thiazol-4-yl)phenyl]-2,6-difluorobenzamide

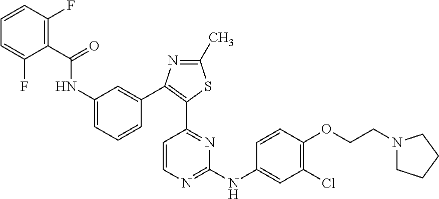

A reaction containing the N-{3-[5-(2-chloro-4-pyrimidinyl)-2-methyl-1,3-thiazol-4-yl]phenyl}-2,6-difluorobenzamide (0.1 g; 0.28 mmol), prepared by a procedure analogous to Example 107, 3-chloro-4-{[2-(1-pyrrolidinyl)ethyl]oxy}phenyl amine (0.027 mmol), prepared by a procedure analogous to Example 27, Step B, in i-PrOH (3 mL) with 3 drops of concentrated HCl was heated with stirring at 90° C. for 16 h. The reaction was concentrated in vacuo to a crude residue. Purification using RP HPLC gave the desired product (0.040 g, 22%). ¹H-NMR (400 mHz, CDCl₃-d) δ 8.18 (d, 1H, J=5.3 Hz), 7.82 (d, 1H, J=8.2 Hz), 7.78 (brs, 1H), 7.76 (d, 1H, J=2.6 Hz), 7.71 (s, 1H), 7.45-7.39 (m, 2H), 7.36-7.3 (m, 2H), 7.04-6.97 (m, 3H), 6.91 (d, 1H, J=5.1 Hz), 6.61 (d, 1H, J=5.1 Hz), 4.24-4.17 (m, 1H), 3.0 (brs, 2H), 2.76 (brs, 7H), and 1.83 (brs, 4H); MS (ESI) 647 [M+H] and 645 [M−H].

### Example 111

### 2,6-Difluoro-N-[3-(5-{2-[(3-fluoro-4-{[1-(1-methylethyl)-4-piperidinyl]oxy}phenyl)amino]-4-pyrimidinyl}-2-methyl-1,3-thiazol-4-yl)phenyl]benzamide

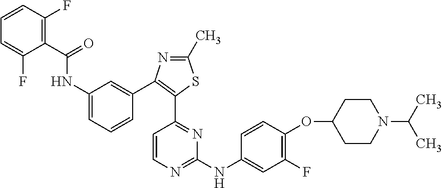

2-Bromopropane (2.85 mmol) was added to a mixture containing 2,6-difluoro-N-{3-[5-(2-{[3-fluoro-4-(4-piperidinyloxy)phenyl]amino}-4-pyrimidinyl)-2-methyl-1,3-thiazol-4-yl]phenyl}benzamide (0.16 mmol), prepared by a procedure analogous to Example 107, and TEA (0.17 mmol) in DMF (3 mL) and the reaction was refluxed for 16 h. The reaction was concentrated in vacuo to a crude residue. Purification by RP HPLC gave the desired product (0.024 g, 23%). ¹H-NMR (400 mHz, DMSO-d₆) δ 10.89 (brs, 1H), 9.74 (s, 1H), 8.31 (d, 1H, J=4.9 Hz), 7.93 (s, 1H), 7.75 (d, 1H, J=7.9 Hz), 7.69 (d, 1H, J=14.1 Hz), 7.43 (t, 1H, J=7.9 Hz), 7.35 (d, 1H, J=8.8 Hz), 7.28-7.20 (m, 3H), 7.08 (t, 1H, J=9.2 Hz), 6.55 (d, 1H, J=5.1 Hz), 4.21-4.15 (m, 1H), 2.73 (s, 3H), 2.71-2.65 (m, 3H), 2.26 (t, 2H, J=9.0 Hz), 1.87 (brs, 2H), 1.65-1.53 (m, 2H), and 0.94 (d, 6H, J=6.4 Hz).

### Example 112

### N-[3-(5-{2-[(4-{[2-(Dimethylamino)ethyl]oxy}-3-fluorophenyl)amino]-4-pyrimidinyl}-2-ethyl-1,3-thiazol-4-yl)phenyl]-2,6-difluorobenzamide

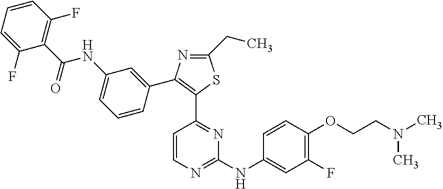

### Step A: N-{3-[5-(2-Chloro-4-pyrimidinyl)-2-ethyl-1,3-thiazol-4-yl]phenyl}-2,6-difluorobenzamide

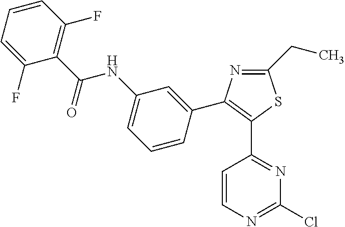

To a solution of N-{3-[(2-chloro-4-pyrimidinyl)acetyl]phenyl}-2,6-difluorobenzamide (1.00 g, 2.58 mmol), prepared by a procedure analogous to Example 1, Step C, in DMF (13 mL) was added NBS (459 mg, 2.58 mmol). The reaction was stirred for 30 min at rt, and then propanethioamide (946 mg, 3.87 mmol) was added. The reaction was stirred a further 30 min at rt and was then diluted with EtOAc (125 mL). The mixture was extracted with H₂O (3×125 mL). The organic fraction was dried over Na₂SO₄, filtered, and concentrated. Purification by flash column chromatography (20 to 70% EtOAc:hexanes) afforded 730 mg (62%) of the title compound of Step A. ¹H-NMR (300 MHz, CDCl₃) δ 8.36 (d, 1H, J=5.3 Hz), 7.80 (m, 2H), 7.74 (brs, 1H), 7.44 (m, 2H), 7.32 (m, 1H), 7.12 (d, 1H, J=5.3 Hz), 7.01 (t, 2H, J=8.2 Hz), 3.08

### Step B: N-[3-(5-{2-[(4-{[2-(Dimethylamino)ethyl]oxy}-3-fluorophenyl)amino]-4-pyrimidinyl}-2-ethyl-1,3-thiazol-4-yl)phenyl]-2,6-difluorobenzamide

The title compound of Example 112 was prepared in 50% yield by a procedure analogous to Example 1, Step D from N-{3-[5-(2-chloro-4-pyrimidinyl)-2-ethyl-1,3-thiazol-4-yl]phenyl}-2,6-difluorobenzamide (100 mg, 0.219 mmol) and 4-{[2-(dimethylamino)ethyl]oxy}-3-fluorophenyl amine hydrochloride (57 mg, 0.24 mmol) in i-PrOH (2.2 mL) at 180° C. under microwave conditions. Purification was performed by preparative HPLC (10 to 70% acetonitrile:H₂O with 0.1% TFA). ¹H-NMR (400 MHz, CDCl₃) δ 8.20 (d, 1H, J=5.7 Hz), 7.82 (m, 2H), 7.65 (d, 1H, J=10.6 Hz), 7.45-7.35 (m, 3H), 7.13 (m, 2H), 6.97 (m, 3H), 6.81 (brs, 1H), 6.64 (d, 1H, J=4.8 Hz), 4.17 (t, 2H, J=5.5 Hz), 3.08 (q, 2H, J=7.1 Hz), 2.84 (m, 2H), 2.43 (s, 6H), and 1.46 (t, 3H, J=7.2 Hz; 620.14 [M+H]⁺.

### Example 113

### N-[3-(5-{2-[(3-Chloro-4-{[2-(1-pwrolidinyl)ethyl]oxy}phenyl)amino]-4-pyrimidinyl}-2-ethyl-1,3-thiazol-4-yl)phenyl]-2,6-difluorobenzamide

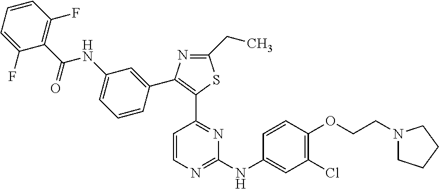

The title compound of Example 113 was synthesized using the standard microwave chloride displacement conditions analogous to Example 1, Step D, in iPrOH using N-{3-[5-(2-chloro-4-pyrimidinyl)-2-ethyl-1,3-thiazol-4-yl]phenyl}-2,6-difluorobenzamide (0.125 g, 0.274 mmol), prepared by a procedure analogous to Example 112, Step A, and 3-chloro-4-{[2-(1-pyrrolidinyl)ethyl]oxy}phenyl amine hydrochloride (0.095 g, 0.34 mmol), prepared by a procedure analogous to Example 27, Step B. The solvent was removed and the residue was taken up in MeOH and purified via HPLC. Desired fractions were combined and dried. The residual material was dissolved in EtOAc (30 mL), washed with saturated aqueous sodium bicarbonate (2×40 mL), dried over sodium sulfate, filtered, and concentrated to give 0.112 g, 62% yield, of the desired product of Example 113 as a solid. ¹H-NMR (400 MHz, DMSO-d₆) δ 10.93 (s, 1H), 9.73 (s, 1H), 8.33 (d, J=4.4 Hz, 1H), 8.33 (d, J=4.4 Hz, 1H), 7.79 (d, J=7.5 Hz, 1H), 7.57 (m, 2H), 7.45 (t, J=7.5 Hz, 1H), 7.28 (m, 3H), 7.08 (d, J=9.2 Hz, 1H), 6.57 (d, J=4.6 Hz, 1H), 4.10 (m, 2H), 3.07 (d, J=7.3 Hz, 2H), 2.80 (m, 2H), 2.55 (m, 4H), 1.68 (s, 4H), and 1.37 (t, J=7.1 Hz, 3H); ES-LCMS m/z 661 (M+H).

### Example 114

### N-[3-(5-{2-[(3-Chloro-4-{[2-(dimethylamino)ethyl]oxy}phenyl)amino]-4-pyrimidinyl}-2-ethyl-1,3-thiazol-4-yl)phenyl]-2,6-difluorobenzamide

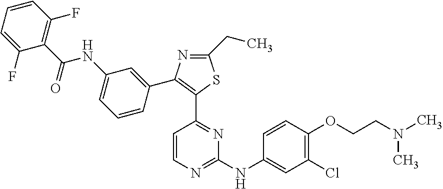

The title compound of Example 111 was prepared in 36% yield by a procedure analogous to Example 1, Step D, from N-{3-[5-(2-chloro-4-pyrimidinyl)-2-ethyl-1,3-thiazol-4-yl]phenyl}-2,6-difluorobenzamide (100 mg, 0.219 mmol), prepared by a procedure analogous to Example 112, Step A, and 4-{[2-(dimethylamino)ethyl]oxy}-3-chlorophenyl amine hydrochloride (61 mg, 0.24 mmol) in i-PrOH (2.2 mL) at 180° C. under microwave conditions. Purification was performed by preparative HPLC (10 to 70% acetonitrile:H₂O with 0.1% TFA). ¹H-NMR (400 MHz, CDCl₃ δ 8.19 (d, 1H, J=5.1 Hz), 7.82 (m, 3H), 7.45-7.34 (m, 4H), 7.15 (s, 1H), 7.00 (t, 2H, J=8.2 Hz), 6.92 (d, 1H, J=8.8 Hz), 6.78 (br s, 1H), 6.63 (d, 1H, J=5.1 Hz), 4.14 (t, 2H, J=5.8 Hz), 3.08 (q, 2H, J=7.7 Hz), 2.80 (t, 2H, J=5.6 Hz), 2.38 (s, 6H), and 1.46 (t, 3H J=7.6 Hz); MS (APCI): 635.11 [M+H]⁺.

### Example 115

### N-{3-[2-Ethyl-5-(2-{[3-(1-pyrrolidinylmethyl)phenyl]amino}-4-pyrimidinyl)-1,3-thiazol-4-yl]phenyl}-2,6-difluorobenzamide

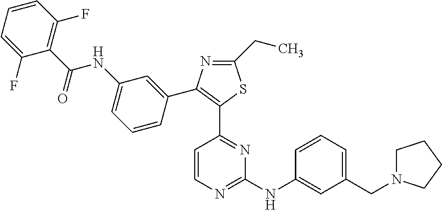

The title compound of Example 115 was prepared in 39% yield by a procedure analogous to Example 1, Step D from N-{3-[5-(2-chloro-4-pyrimidinyl)-2-ethyl-1,3-thiazol-4-yl]phenyl}-2,6-difluorobenzamide (100 mg, 0.219 mmol), prepared by a procedure analogous to Example 112, Step A, and 3-(1-pyrrolidinylmethyl)phenyl amine (42 mg, 0.24 mmol) in i-PrOH (2.2 mL) at 180° C. under microwave conditions. Purification was performed by preparative HPLC (10 to 70% acetonitrile:H₂O with 0.1% TFA). ¹H-NMR (400 MHz, CDCl₃) δ 8.20 (d, 1H, J=5.1 Hz), 7.88 (d, 1H, J=8.1 Hz), 7.75 (m, 2H), 7.58 (m, 2H), 7.43 (m, 2H), 7.36 (m, 1H), 7.30 (t, 1H, J=8.2 Hz), 7.21 (s, 1H), 7.01 (m, 3H), 6.61 (d, 1H, J=4.9 Hz), 3.65 (s, 2H), 3.08 (q, 2H, J=7.5 Hz), 2.55 (s, 4H), 1.79 (s, 4H), and 1.46 (t, 3H, J=7.6 Hz); MS (APCI): 597.28 [M+H]⁺.

### Example 116

### N-{3-[5-(2-{[4-(1,4′-Bipiperidin-1′-yl)-3-fluorophenyl]amino}-4-pyrimidinyl)-2-ethyl-1,3-thiazol-4-yl]phenyl}-2,6-difluorobenzamide

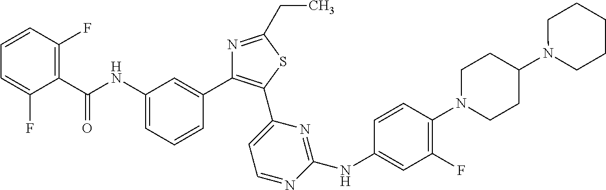

### Step A: 1′-(2-Fluoro-4-nitrophenyl)-1,4′-bipiperidine

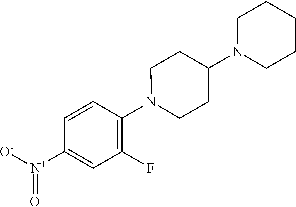

To a solution containing 2.0 mL (18.1 mmol) of 1,2-difluoro-4-nitrobenzene and 20 mL of THF was added 3.3 g (19.9 mmol) of 1,4′-bipiperidine and 5.8 mL (41.6 mmol) of TEA. The reaction mixture was allowed to stir at rt overnight and partitioned between EtOAc and H₂O. The aqueous layer was further extracted with EtOAc and the combined organic layers were dried over MgSO₄. The solvent was removed under reduced pressure and the residue was subjected to silica gel chromatography to give 3.9 g (68%) of 1′-(2-fluoro-4-nitrophenyl)-1,4′-bipiperidine as a bright yellow solid: ¹H-NMR (400 MHz, DMSO-d₆) δ7.98 (d, J=11.4 Hz, 2H), 7.15 (t, J=9.1 Hz, 1H), 3.72 (d, J=12.1 Hz, 2H), 2.90 (t, J=12.2 Hz, 2H), 2.40-2.48 (m, 5H), 1.81 (d, J=14.1 Hz, 2H), 1.55 (dd, J=12.1 and 3.3 Hz, 2H), 1.45-1.51 (m, 4H), and 1.37 (dt, J=10.7 and 5.3 Hz, 2H); MS (ESI): 308.17 (M+H⁺).

### Step B: 4-(1,4′-Bipiperidin-1′-yl)-3-fluorophenyl amine hydrochloride

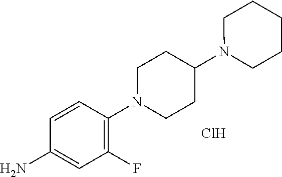

A mixture containing 3.9 g (12.6 mmol) of 1-(2-fluoro-4-nitrophenyl)-1,4′-bipiperidine, 0.4 g of 5% Pt on carbon, and 50 mL of EtOH was subjected to a 50 psi H₂ atmosphere for 5 h. The reaction mixture was filtered through a pad of Celite, eluting with EtOH and EtOAc, and the solvent was removed under reduced pressure. The residue was taken up in EtOAc and 3.2 mL (12.6 mmol) of a 4.0 M solution of HCl in ether was added. The resulting mixture was filtered to give 3.95 g (100%): ¹H-NMR (400 MHz, DMSO-d₆) δ 9.82 (brs, 1H), 6.80 (dd, J=9.9 and 8.8 Hz, 1H), 6.32-6.41 (m, 2H), 5.46 (brs, 2H), 3.42 (d, J=13.0 Hz, 2H), 3.34 (brs, 2H), 3.15-3.25 (m, 3H), 2.86-2.97 (m, 2H), 2.60 (t, J=11.3 Hz, 2H), 2.09 (d, J=11.0 Hz, 2H), and 1.72-1.85 (m, 6H).

### Step C: N-{3-[5-(2-{[4-(1,4′-Bipiperidin-1′-yl)-3-fluorophenyl]amino}-4-pyrimidinyl)-2-ethyl-1,3-thiazol-4-yl]phenyl}-2,6-difluorobenzamide

To a suspension containing 0.1 g (0.2 mmol) of N-{3-[5-(2-chloro-4-pyrimidinyl)-2-ethyl-1,3-thiazol-4-yl]phenyl}-2,6-difluorobenzamide, prepared by a procedure analogous to Example 112, Step A, 0.08 g (0.24 mmol) of 4-(1,4′-bipiperidin-1′-yl)-3-fluorophenyl amine hydrochloride and 2 mL of i-PrOH was added 0.1 mL of a 4.0 M solution of HCl in dioxane. The reaction mixture was heated at 90° C. in a sealed tube for 3 days and the solvent was removed under reduced pressure. The residue was purified by HPLC and further purified by trituration to give 46 mg (30%) of N-{3-[5-(2-{[4-(1,4′-bipiperidin-1′-yl)-3-fluorophenyl]amino}-4-pyrimidinyl)-2-ethyl-1,3-thiazol-4-yl]phenyl}-2,6-difluorobenzamide as a yellow solid: ¹H-NMR (400 MHz, DMSO-d₆) δ 10.92 (s, 1H); 9.73 (s, 1H), 8.33 (d, J=5.1 Hz, 1H), 7.94 (s, 1H), 7.79 (d, J=7.7 Hz, 1H), 7.55-7.66 (m, 2H), 7.44 (t, J=8.0 Hz, 1H), 7.28-7.37 (m, 2H), 7.22-7.28 (m, 2H), 6.95 (t, J=9.9 Hz, 1H), 6.57 (d, J=5.1 Hz, 1H), 3.50 (brs, 1H), 3.29 (brs, 1H), 3.07 (q, J=7.5 Hz, 2H), 2.59 (t, J=11.1 Hz, 2H), 2.26-2.38 (m, 2H), 1.73-1.83 (m, 2H), 1.54-1.66 (m, 2H), 1.46-1.53 (m, 4H), 1.35 1.40 (m, 5H), 1.16-1.26 (m, 2H), and 1.04 (d, J=6.8 Hz, 2H); HRMS calcd for C₃₈H₃₉F₃N₇OS (M+H⁺): 689.2883. Found: 698.2885.

### Example 117

### N-[3-(2-Ethyl-5-{2-[(3-fluoro-4-{[2-(1-pyrrolidinyl)ethyl]oxy}phenyl)amino]-4-pyrimidinyl}-1,3-thiazol-4-yl)phenyl]-2,6-difluorobenzamide

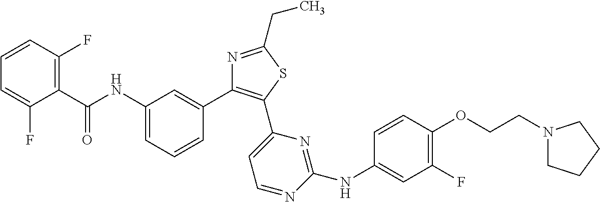

To a solution containing 100 mg (0.22 mmol) of N-{3-[5-(2-chloro-4-pyrimidinyl)-2-ethyl-1,3-thiazol-4-yl]phenyl}-2,6-difluorobenzamide, prepared by a procedure analogous to Example 112, Step A, 63 mg (0.24 mmol) of 3-fluoro-4-{[2-(1-pyrrolidinyl)ethyl]oxy}phenyl amine, prepared by a procedure analogous to Example 33, Step B, and 2 mL of i-PrOH was added 0.1 mL of a 4.0 M solution of HCl in dioxane. The reaction mixture was heated at 90° C. in a sealed tube overnight and the solvents were removed under reduced pressure. The residue was purified by HPLC to give 81 mg (57% of the title compound of Example 117 as a yellow solid: ¹H-NMR (400 MHz, DMSO-d₆) δ 10.92 (s, 1H), 9.74 (s, 1H), 8.33 (d, J=5.3 Hz, 1H), 7.93 (s, 1H), 7.79 (d, J=9.5 Hz, 1H), 7.67-7.74 (m, 1H), 7.54-7.63 (m, 1H), 7.41-7.48 (m, 1H), 7.38 (d, J=8.4 Hz, 1H), 7.21-7.32 (m, 3H), 7.09 (t, J=9.3 Hz, 1H), 6.57 (d, J=6.2 Hz, 1H), 4.09 (t, J=5.7 Hz, 2H), 3.29-3.31 (m, 2H), 3.07 (q, J=7.6 Hz, 2H), 2.78 (t, J=5.3 Hz, 2H), 1.67 (q, J=6.3 Hz, 4H), and 1.37 (t, J=7.5 Hz, 3H); HRMS calcd for C₃₄H₃₂F₃N₆O₂S (M+H⁺): 645.2254. Found: 645.2255.

### Example 118

### N-{3-[2-Ethyl-5-(2-{[6-(4-morpholinyl)-3-pyridinyl]amino}-4-pyrimidinyl)-1,3-thiazol-4-yl]phenyl}-2,6-difluorobenzamide trifluoroacetate

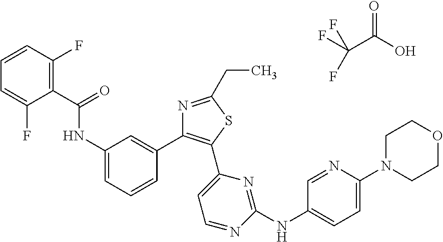

### Step A: 4-(5-Nitro-2-pyridinyl)morpholine

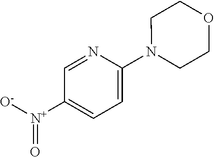

To a solution containing 1.2 mL (13.9 mmol) of morpholine and 20 mL of THF was added 0.66 g (16.4 mmol) of a 60% dispersion of sodium hydride in mineral oil. The reaction was allowed to stir for 15 min and 2.0 g (12.6 mmol) of 2-chloro-5-nitropyridine was added. The reaction mixture was heated at 50° C. overnight, then quenched by the addition of H₂O and extracted with DCM. The combined organic layers were dried over MgSO₄ and the solvents were removed under reduced pressure. The residue was subjected to silica gel chromatography to give 1.65 g (63%) of 4-(5-nitro-2-pyridinyl)morpholine as a yellow solid: ¹H-NMR (400 MHz, DMSO-d₆) δ 8.97 (d, J=2.9 Hz, 1H), 8.25 (dd, J=9.5, 2.9 Hz, 1H), 6.95 (d, j=9.5 Hz, 1H), 3.72-3.75 (m, 4H), and 3.68-3.71 (m, 4H); MS (ESI): 210.28 (M+H⁺).

### Step B: 6-(4-Morpholinyl)-3-pyridinamine

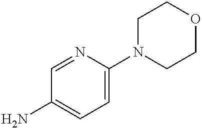

A mixture containing 1.65 g (7.8 mmol) of 4-(5-nitro-2-pyridinyl)morpholine, 160 mg of 5% Pt on carbon, and 20 mL of EtOHI was subjected to a 50 psi H₂ atmosphere for 5 h. The reaction mixture was filtered through a pad of Celite and the solvents were removed under reduced pressure to give 1.4 g (100%) of 6-(4-morpholinyl)-3-Pyridinamine as a purple solid: ¹H-NMR (400 MHz, DMSO-d₆) δ 7.60 (d, J=2.9 Hz, 1H), 6.92 (dd, J=8.8 and 2.9 Hz, 1H), 6.62 (d, J=8.8 Hz, 1H), 4.59 (brs, 2H), 3.65-3.72 (m, 4H), and 3.17 (dt, J=4.9 and 2.4 Hz, 4H).

### Step C: N-{3-[2-Ethyl-5-(2-{[6-(4-morpholinyl)-3-pyridinyl]amino}-4-pyrimidinyl)-1,3-thiazol-4-yl]phenyl}-2,6-difluorobenzamide trifluoroacetate

N-{3-[5-(2-Chloro-4-pyrimidinyl)-2-ethyl-1,3-thiazol-4-yl]phenyl}-2,6-difluorobenzamide (100 mg, 0.22 mmol), prepared by a procedure analogous to Example 112, Step A, was combined with 6-(4-morpholinyl)-3-pyridinamine (36 mg, 0.20 mmol), para-toluenesulfonic acid monohydrate (45 mg, 0.24 mmol) and 2,2,2-trifluoroethanol (3 mL) in a sealed vessel. The reaction was heated for 60 min at 170° C. by microwave radiation. The reaction was cooled and treated with NaHCO₃ (200 mg). The reaction was stirred for 15 min before being concentrated to a residue and purified by silica gel chromatography (gradient: 5-100% (80% CH₂Cl₂:19% MeOH:1% NH₄OH)/CH₂Cl₂). The fractions with coupled adduct were combined and concentrated under vacuum to a brown oil. The residue was purified a second time by RP HPLC (C18; 10-100% acetonitrile (0.1% TFA)/H₂O (0.1% TFA)). The second purification yielded 6 mg (4%) of the title compound of Example 118 as the trifluoroacetate salt. ¹H-NMR (400 MHz, MeOH-d₄) δ 8.69 (d, J=2.6 Hz, 1H), 8.30 (d, J=5.3 Hz, 1H), 8.10 (dd, J=9.8, and 2.5 Hz, 1H), 7.91 (s, 1H), 7.65-7.71 (m, 1H), 7.43-7.54 (m, 2H), 7.35 (s, 1H), 7.30-7.34 (m, 1H), 7.07 (t, J=8.1 Hz, 2H), 6.71 (d, J=5.3 Hz, 1H), 3.82-3.89 (m, 4H), 3.56-3.63 (m, 4H), 3.08 (q, J=7.6 Hz, 2H), and 1.44 (t, J=7.5 Hz, 3H); m/z (ESI): 600.15 [M+H]⁺.

### Example 119

### N-[3-(5-{2-[(3-Chloro-4-{[2-(1-pyrrolidinyl)ethyl]oxy}phenyl)amino]-4-pyrimidinyl}-2-{[1,1-dimethylethyl)sulfonyl]methyl}-1,3-thiazol-4-yl)phenyl]-2,6-difluorobenzamide

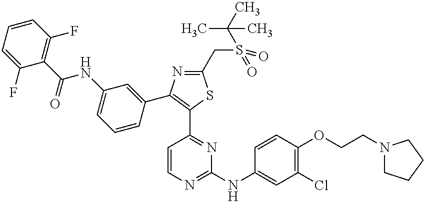

### Step A: N-[3-(5-(2-Chloro-4-pyrimidinyl)-2-{[(1,1-dimethylethyl)sulfonyl]methyl}-1,3-thiazol-4-yl)phenyl]-2,6-difluorobenzamide

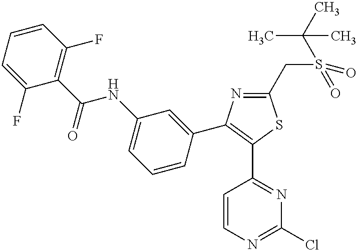

The title compound of Step A was prepared in 66% yield from N-{3-[(2-chloro-4-pyrimidinyl)acetyl]phenyl}-2,6-difluorobenzamide (1.00 g, 2.58 mmol), prepared by a procedure analogous to Example 112, Step A, and 2-[(1,1-dimethylethyl)sulfonyl]-ethanethioamide (1.01 g, 5.16 mmol), by a procedure analogous to Example 1, Step C. ¹H-NMR (300 MHz, CDCl₃) δ 8.41 (d, 1H, J=5.2 Hz), 7.83 (t, 1H, J=1.7 Hz), 7.79 (m, 2H), 7.45 (m, 2H), 7.31 (m, 1H), 7.15 (d, 1H, J=5.3 Hz), 7.01 (m, 2H), 4.69 (s, 2H), and 1.48 (s, 5H); MS (ESI): 563.06 [M+H]⁺.

### Step B: N-[3-(5-{2-[(3-Chloro-4-{[2-(1-pyrrolidinyl)ethyl]oxy}phenyl)amino]-4-pyrimidinyl}-2-{[(1,1-dimethylethyl)sulfonyl]methyl}-1,3-thiazol-4-yl)phenyl]-2,6-difluorobenzamide

The title compound of Example 119 was prepared in 53% yield by a procedure analogous to Example 1, Step D from N-[3-(5-(2-chloro-4-pyrimidinyl)-2-{[(1,1-dimethylethyl)sulfonyl]methyl}-1,3-thiazol-4-yl)phenyl]-2,6-difluorobenzamide (100 mg, 0.178 mmol) and 3-chloro-4-{[2-(1-pyrrolidinyl)ethyl]oxy}phenyl amine hydrochloride (54 mg, 0.20 mmol), prepared by a procedure analogous to Example 27, Step B, in i-PrOH (1.8 mL) at 180° C. under microwave conditions. Purification was performed by preparative HPLC (10 to 70% acetonitrile:H₂O with 0.1% TFA). ¹H-NMR (400 MHz, CDCl₃) δ 8.23 (d, 1H, J=5.7 Hz), 7.81 (m, 3H), 7.69 (s, 1H), 7.45-7.35 (m, 4H), 7.15 (s, 1H), 7.02-6.91 (m, 3H), 6.64 (d, 1H, J=4.4 Hz), 4.71 (s, 2H), 4.18 (t, 2H, J=5.9 Hz), 2.96 (t, 2H, J=5.6 Hz), 2.68 (s, 4H), 1.81 (s, 4H), and 1.48 (s, 9H); MS (APCI): 768.35 [M+H]⁺.

### Example 120

### N-[3-(2-{[(1,1-Dimethylethyl)sulfonyl]methyl}-5-{2-[(2-methyl-1,2,3,4-tetrahydro-7-isoquinolinyl)amino]-4-pyrimidinyl}-1,3-thiazol-4-yl)phenyl]-2,6-difluorobenzamide

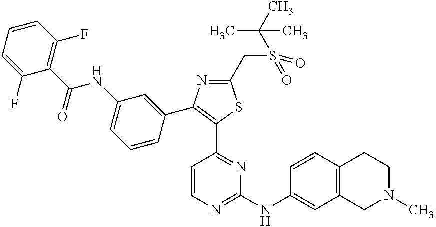

The title compound of Example 120 was prepared in 37% yield by a procedure analogous to Example 1, Step D, from N-[3-(5-(2-chloro-4-pyrimidinyl)-2-([(1,1-dimethylethyl)sulfonylimethyl]-1,3-thiazol-4-yl)phenyl]-2,6-difluorobenzamide (100 mg, 0.178 mmol), prepared by a procedure analogous to Example 119, Step A, and 2-methyl-1,2,3,4-tetrahydro-7-isoquinolinamine (32 mg, 0.20 mmol) in i-PrOH (1.8 mL) at 180° C. under microwave conditions. Purification was performed by preparative HPLC (10 to 70% acetonitrile:H₂O with 0.1% TFA). ¹H-NMR (400 MHz, CDCl₃) δ 8.21 (d, 1H, J=5.1 Hz), 8.03 (s, 1H), 7.83 (d, 1H, J=7.7 Hz), 7.75 (s, 1H), 7.46-7.38 (m, 3H), 7.31 (m, 1H), 7.25 (m, 1H), 7.14 (s, 1H), 7.07-6.98 (m, 3H), 6.60 (d, 1H, J=5.1 Hz), 4.71 (s, 2H), 3.62 (s, 2H), 2.89 (t, 2H, J=5.3 Hz), 2.66 (t, J=5.9 Hz), 2.44 (s, 3H), and 1.47 (s, 9H); MS (ESI): 689.16 [M+H]⁺.

### Example 121

### N-{3-[5-{2-[(3-chloro-4-{[2-(1-pyrrolidinyl)-ethyl]oxy}phenyl)amino]-4-pyrimidinyl}-2-(1-methylethyl)-1,3-thiazol-4-yl]phenyl}-2,6-difluorobenzamide

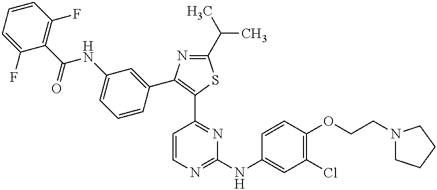

### Step A: 2-Methylpropanethioamide

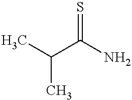

A solution of 2-methylpropanamide (6.53 g, 75.0 mmol) and 2,4-bis(4-methoxyphenyl)-1,3-dithia-2,4-diphosphetane-2,4-disulfide (15.17 g, 37.51 mmol) in THF (100 mL) was heated to reflux for 4 h. The reaction mixture was then cooled to rt and poured into saturated aqueous NaHCO₃ (200 mL). The mixture was extracted with ether (4×100 mL). The organic fractions were combined, dried over Na₂SO₄, filtered, and concentrated. Purification by flash column chromatography (20% EtOAc:hexanes) afforded 4.77 g (62%) of the title compound of Step A. ¹H-NMR (400 MHz, CDCl₃) δ 7.63 (brs, 1H), 6.90 (brs, 1H), 2.88 (m, 1H), and 1.27 (d, 6H, J=6.8 Hz).

### Step B: N-{3-[5-(2-Chloro-4-pyrimidinyl)-2-(1-methylethyl)-1,3-thiazol-4-yl]phenyl}-2,6-difluorobenzamide

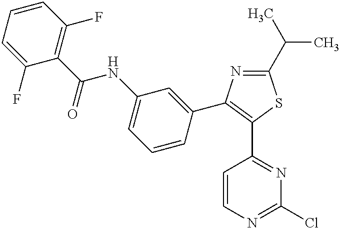

The title compound of Step B was prepared (0.93 g, 61%) from N-{3-[(2-chloro-4-pyrimidinyl)acetyl]phenyl}-2,6-difluorobenzamide (1.25 g, 3.22 mmol), prepared by a procedure analogous to Example 1, Step C, and 2-methylpropanethioamide (498 mg, 4.83 mmol), by a procedure analogous to Example 112, Step A. ¹H-NMR (400 MHz, CDCl₃) δ 8.35 (d, 1H, J=5.5 Hz), 7.80 (m, 2H), 7.72 (s, 1H), 7.44 (m, 2H), 7.32 (d, 1H, J=7.9 Hz), 7.11 (d, 1H, J=5.3 Hz), 7.01 (t, 2H, J=8.2 Hz), 3.36 (m, 1H), and 1.46 (d, 6H, J=7.0 Hz); MS (ESI): 471.09 [M+H]⁺.

### Step C: N-{3-[5-{2-[(3-Chloro-4-{[2-(1-pyrrolidinyl)ethyl]oxy}phenyl)amino]-4-pyrimidinyl}-2-(1-methylethyl)-1,3-thiazol-4-yl]phenyl}-2,6-difluorobenzamide

The title compound of Example 121 was prepared in 51% yield by a procedure analogous to Example 1, Step D from N-{3-[5-(2-chloro-4-pyrimidinyl)-2-(1-methylethyl)-1,3-thiazol-4-yl]phenyl}-2,6-difluorobenzamide (100 mg, 0.212 mmol) and 3-chloro-4-{[2-(1-pyrrolidinyl)ethyl]oxy}phenyl amine hydrochloride (62 mg, 0.22 mmol), prepared by a procedure analogous to Example 27, Step B, in i-PrOH (2.1 mL) at 180° C. under microwave conditions. Purification was performed by preparative HPLC (10 to 70% acetonitrile:H₂O with 0.1% TFA). ¹H-NMR (400 MHz, CDCl₃) δ 8.18 (d, 1H, J=4.0 Hz), 7.87-7.78 (m, 4H), 7.42-7.28 (m, 4H), 7.18 (s, 1H), 7.02-6.90 (m, 3H), 6.62 (d, 1H, J=2.9 Hz), 4.17 (t, 2H, J=5.6 Hz), 3.35 (m, 1H), 2.96 (t, 2H, J=5.3 Hz), 2.68 (s, 4H), 1.81 (s, 4H), and 1.47 (d, 6H, J=6.4 Hz); MS (APCI): 675.53 [M+H]⁺.

### Example 122

### N-{3-[5-{2-[(3-{[2-(Dimethylamino)ethyl]oxy}phenvflamino]-4-pyrimidinyl}-2-(1-methylethyl)-1,3-thiazol-4-yl]phenyl}-2,6-difluorobenzamide

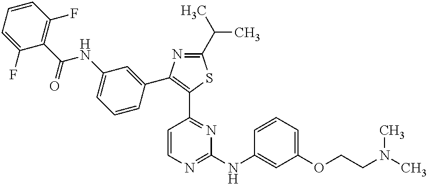

The title compound of Example 122 was prepared (0.13 g, 23%) by a procedure analogous to Example 1, Step D, from N-{3-[5-(2-chloro-4-pyrimidinyl)-2-(1-methylethyl)-1,3-thiazol-4-yl]phenyl}-2,6-difluorobenzamide (100 mg, 0.212 mmol), prepared by a procedure analogous to Example 121, Step B, and 3-{[2-(dimethylamino)ethyl]oxy}phenyl amine dihydrochloride (56 mg, 0.22 mmol) in i-PrOH (2.1 mL) at 180° C. under microwave conditions. Purification was performed by preparative HPLC (10 to 70% acetonitrile:H₂O with 0.1% TFA). ¹H-NMR (400 MHz, CDCl₃) δ 8.18 (d, 1H, J=4.9 Hz), 7.86-7.73 (m, 3H), 7.48-7.32 (m, 3H), 7.21 (m, 3H), 7.04-6.96 (m, 3H), 6.60 (m, 2H), 4.11 (t, 2H, J=5.3 Hz), 3.34 (m, 1H), 2.73 (t, 2H, J=5.0 Hz), 2.32 (s, 6H), and 1.45 (d, 6H, J=6.8 Hz); MS (APCI): 616.11 [M+H]⁺.

### Example 123

### N-{3-[2-(1,1-Dimethylethyl)-5-(2-{[3-(1-pyrrolidinylmethyl)phenyl]amino}-4-pyrimidinyl)-1,3-thiazol-4-yl]phenyl}-2,6-difluorobenzamide

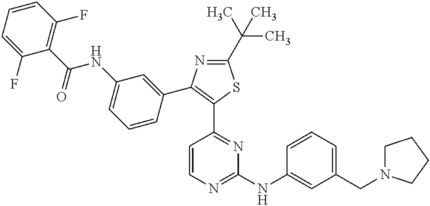

### Step A: 2,2-Dimethylpropanethioamide

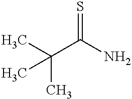

The title compound of Step A was prepared (3.2 g, 36%) from 2,2-dimethylpropanamide (7.59 g, 75.0 mmol) and 2,4-bis(4-methoxyphenyl)-1,3-dithia-2,4-diphosphetane-2,4-disulfide (15.17 g, 37.51 mmol) by a procedure analogous to Example 121, Step A. ¹H-NMR (400 MHz, CDCl₃) δ 7.92 (brs, 1H), 7.03 (brs, 1H), and 1.38 (s, 9H).

### Step B: N-{3-[5-(2-Chloro-4-pyrimidinyl)-2-(1,1-dimethylethyl)-1,3-thiazol-4-yl]phenyl}-2,6-difluorobenzamide

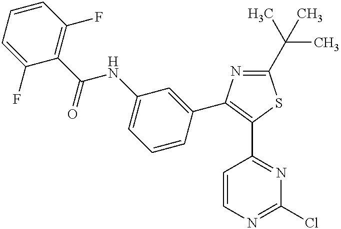

The title compound of Step B was prepared (1.1 g, 70%) from N-{3-[(2-chloro-4-pyrimidinyl)acetyl]phenyl}-2,6-difluorobenzamide (1.25 g, 3.22 mmol) and 2,2-dimethylpropanethioamide (566 mg, 4.83 mmol), prepared by a procedure analogous to Example 1, Step C, by a procedure analogous to Example 112, Step A. ¹H-NMR (400 MHz, CDCl₃) δ 8.35 (d, 1H, J=5.3 Hz), 7.80 (m, 2H), 7.71 (s, 1H), 7.44 (m, 2H), 7.33 (d, 1H, J=7.5 Hz), 7.09 (d, 1H, J=5.3 Hz), 7.01 (t, 2H, J=8.3 Hz), and 1.50 (s, 9H); MS (APCI): 485.14 [M+H]⁺.

### Step C: N-{3-[2-(1,1-Dimethylethyl)-5-(2-{[3-(1-pyrrolidinylmethyl)phenyl]amino}-4-pyrimidinyl)-1,3-thiazol-4-yl]phenyl}-2,6-difluorobenzamide

The title compound of Example 123 was prepared (129 mg, 34%) by a procedure analogous to Example 1, Step D, from N-{3-[5-(2-chloro-4-pyrimidinyl)-2-(1,1-dimethylethyl)-1,3-thiazol-4-yl]phenyl}-2,6-difluorobenzamide (100 mg, 0.206 mmol) and 3-(1-pyrrolidinylmethyl)phenyl amine (38 mg, 0.22 mmol) in i-PrOH (2.1 mL) at 180° C. under microwave conditions. Purification was performed by preparative HPLC (10 to 70% acetonitrile:H₂O with 0.1% TFA). ¹H-NMR (400 MHz, CDCl₃) δ 8.19 (d, 1H, J=5.1 Hz), 7.89 (d, 1H, J=8.4 Hz), 7.73 (m, 2H), 7.63 (s, 1H), 7.51 (d, 1H, J=7.5 Hz), 7.40 (m, 3H), 7.28 (m, 1H), 7.21 (s, 1H), 7.05-6.98 (m, 3H), 6.59 (d, 1H, J=4.9 Hz), 3.65 (s, 2H), 2.54 (s, 4H), 1.79 (s, 4H), and 1.51 (s, 9H); MS (ESI): 625.32 [M+H]⁺.

### Example 124

### 2,6-Difluoro-N-{3-[5-{2-[(2-methyl-1,2,3,4-tetrahydro-7-isoquinolinyl)amino]-4-pyrimidinyl}-2-(4-piperidinyl)-1,3-thiazol-4-yl]phenyl}benzamide

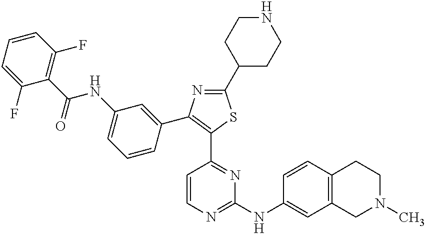

### Step A: 1,1-Dimethylethyl 4-[5-(2-chloro-4-pyrimidinyl)-4-(3-{[(2,6-difluorophenyl)carbonyl]amino}phenyl)-1,3-thiazol-2-yl]-1-piperidinecarboxylate

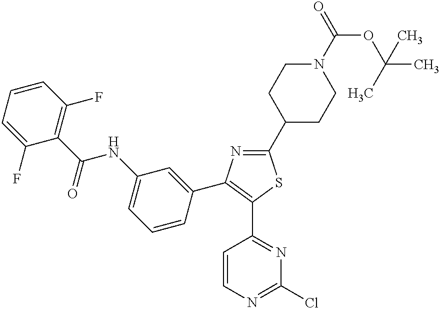

The title compound of Step A was prepared (3.16 g, 80%) (76% purity by LC/MS analysis) from N-{3-[(2-chloro-4-pyrimidinyl)acetyl]phenyl}-2,6-difluorobenzamide (2.50 g, 6.45 mmol), prepared by a procedure analogous to Example 1, Step C, and 1,1-dimethylethyl 4-(aminocarbonothioyl)-1-piperidinecarboxylate (2.36 g, 9.67 mmol), by a procedure analogous to Example 112, Step A. MS (APCI): 611.99 [M+H]⁺.

### Step B: 2,6-Difluoro-N-{3-[5-{2-[(2-methyl-1,2,3,4-tetrahydro-7-isoquinolinyl)amino]-4-pyrimidinyl}-2-(4-piperidinyl)-1,3-thiazol-4-yl]phenyl}benzamide

The title compound of Example 124 was prepared (44 mg, 34%) by a procedure analogous to Example 1, Step D, from 1,1-dimethylethyl 4-[5-(2-chloro-4-pyrimidinyl)-4-(3-{[(2,6-difluorophenyl)carbonyl]amino}phenyl)-1,3-thiazol-2-yl]-1-piperidinecarboxylate (125 mg, 0.204 mmol) and 2-methyl-1,2,3,4-tetrahydro-7-isoquinolinamine (35 mg, 0.21 mmol) in i-PrOH (2.0 mL) at 180° C. under microwave conditions. The BOC group was completely removed under these reaction conditions. Purification was performed by preparative HPLC (10 to 70% acetonitrile:H₂O with 0.1% TFA). ¹H-NMR (400 MHz, DMSO-d₆) δ 10.93 (s, 1H), 9.61 (s, 1H), 8.31 (d, 1H, J=5.1 Hz), 7.94 (t, 1H, J=1.8 Hz), 7.80 (m, 1H), 7.02-7.55 (m, 2H), 7.45 (t, 1H, J=8.0 Hz), 7.38-7.24 (m, 4H), 6.99 (d, 1H, J=8.2 Hz), 6.56 (d, 1H, J=5.3 Hz), 3.45 (s, 2H), 3.13 (m, 1H), 3.03 (m, 2H), 2.75 (t, 2H, J=5.6 Hz), 2.65-2.56 (m, 4H), 2.33 (s, 3H), 2.05 (m, 2H), and 1.62 (m, 2H); MS (APCI): 638.15 [M+H]⁺.

### Example 125

### 2,6-Difluoro-N-[3-(2-(1-methyl-4-piperidinyl)-5-{2-[(2-methyl-1,2,3,4-tetrahydro-7-isoquinolinyl)amino]-4-pyrimidinyl}-1,3-thiazol-4-yl)phenyl]benzamide

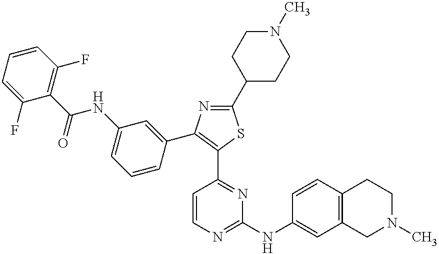

To a solution of 2,6-difluoro-N-{3-[5-{2-[(2-methyl-1,2,3,4-tetrahydro-7-isoquinolinyl)amino]-4-pyrimidinyl}-2-(4-piperidinyl)-1,3-thiazol-4-yl]phenyl}benzamide (220 mg, 0.375 mmol) (prepared in a manner analogous to Example 124, Step B) in MeOH (2.5 mL) and DCM (5.0 mL) was added formaldehyde (37 wt % aqueous solution, 33 μL, 0.45 mmol), HOAc (26 μL, 0.45 mmol), and sodium triacetoxyborohydride (119 mg, 0.561 mmol). The reaction was stirred for 30 min at rt and was then concentrated. The mixture was purified by preparative HPLC (10 to 70% acetonitrile:H₂O with 0.1% TFA). The purified material was converted to the free base by dissolving in EtOAc (30 mL) and washing with saturated aqueous NaHCO₃ (2×50 mL). The organic fraction was dried over Na₂SO₄, filtered, and concentrated to afford 96 mg (43%) of the title compound of Example 125. ¹H-NMR (400 MHz, DMSO-d₆) δ 10.93 (s, 1H), 9.61 (s, 1H), 8.31 (d, 1H, J=5.3 Hz), 7.94 (t, 1H, J=1.7 Hz), 7.81 (m, 1H), 7.63-7.54 (m, 2H), 7.45 (t, 1H, J=8.0 Hz), 7.38-7.24 (m, 4H), 6.99 (d, 1H, J=8.4 Hz), 6.56 (d, 1H, J=5.3 Hz), 3.45 (s, 2H), 3.01 (m, 1H), 2.85 (m, 2H), 2.75 (t, 2H, J=5.7 Hz), 2.57 (t, 2H, J=5.8 Hz), 2.33 (s, 3H), 2.20 (s, 3H), 2.11-2.02 (m, 4H), and 1.78 (m, 2H); MS (APCI): 652.19 [M+H]⁺.

### Example 126

### 2,6-Difluoro-N-{3-[5-{2-[(3-fluoro-4-{[2-(1-pyrrolidinyl)ethyl]oxy}phenyl)amino]-4-pyrimidinyl}-2-(hydroxymethyl)-1,3-thiazol-4-yl]phenyl}benzamide

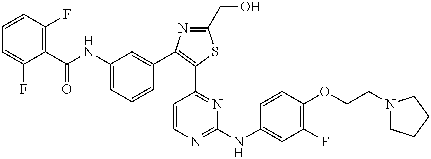

### Step A: [5-(2-Chloro-4-pyrimidinyl)-4-(3-{[(2,6-difluorophenyl)carbonyl]amino}phenyl)-1,3-thiazol-2-yl]methyl 2,2-dimethylpropanoate

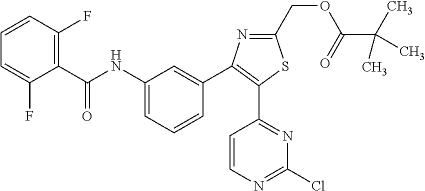

The title compound of Step A was prepared (1.45 g, 53%) from N-{3-[(2-chloro-4-pyrimidinyl)acetyl]phenyl}-2,6-difluorobenzamide, prepared by a procedure analogous to Example 1, Step C, (1.00 g, 2.58 mmol) and 2-amino-2-thioxoethyl 2,2-dimethylpropanoate (904 mg, 5.16 mmol) by a procedure analogous to Example 112, Step A, except that the flash column chromatography gradient was run from 25 to 100% EtOAc:hexanes. ¹H-NMR (300 MHz, CDCl₃) δ 8.40 (d, 1H, J=5.3 Hz), 7.81 (m, 2H), 7.71 (brs, 1H), 7.50-7.39 (m, 2H), 7.33 (dt, 1H, J=1.3 and 7.7 Hz), 7.16 (d, 1H, J=5.3 Hz), 7.02 (m, 2H), 5.41 (s, 2H), and 1.32 (s, 9H); MS (APCI): 565.14 [M+H]⁺.

### Step B: (4-(3-{[(2,6-Difluorophenyl)carbonyl]amino}phenyl)-5-{2-[(3-fluoro-4-{[2-(1-pyrrolidinyl)ethyl]oxy}phenyl)amino]-4-pyrimidinyl}-1,3-thiazol-2-yl)methyl 2,2-dimethylpropanoate

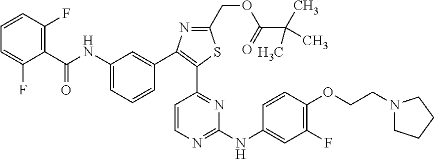

The title compound of Step B was prepared from [5-(2-chloro-4-pyrimidinyl)-4-(3-{[(2,6-difluorophenyl)carbonyl]amino}phenyl)-1,3-thiazol-2-yl]methyl 2,2-dimethylpropanoate (125 mg, 0.230 mmol) and 3-fluoro-4-{[2-(1-pyrrolidinyl)ethyl]oxy}phenyl amine hydrochloride (75 mg, 0.288 mmol), prepared by a procedure analogous to Example 26, Step E, in ˜100% crude yield (220 mg) and 90% purity, by a procedure analogous to Example 1, Step D. No purification was performed at this stage, and the material was carried on crude into the next step. MS (APCI): 731.27 [M+H]⁺.

### Step C: 2,6-Difluoro-N-{3-[5-{2-[(3-fluoro-4-{[2-(1-pyrrolidinyl)ethyl]oxy}phenyl)amino]-4-pyrimidinyl}-2-(hydroxymethyl)-1,3-thiazol-4-yl]phenyl}benzamide

To a solution of crude (4-(3-{[(2,6-difluorophenyl)carbonyl]amino}phenyl)-5-{2-[(3-fluoro-4-{[2-(1-pyrrolidinyl)ethyl]oxy}phenyl)amino]-4-pyrimidinyl}-1,3-thiazol-2-yl)methyl 2,2-dimethylpropanoate (˜168 mg, ˜0.230 mmol) in MeOH (2.3 mL) was added a solution of sodium methoxide in MeOH (25 wt %, 263 μL, 1.15 mmol). The reaction was stirred for 30 min at rt and was then poured into a mixture of H₂O (25 mL) and saturated aqueous NaHCO₃ (25 mL). The aqueous layer was extracted with EtOAc (2×20 mL). The combined organic fractions were dried over Na₂SO₄, filtered, concentrated, and purified by preparative HPLC (10 to 70% acetonitrile:H₂O w/0.1% TFA). The material obtained was redissolved in EtOAc (30 mL) and washed with saturated aqueous NaHCO₃ (2×50 mL). The organic fraction was dried over Na₂SO₄, filtered, and concentrated to afford 81 mg (60%) of the title compound of Example 126. ¹H-NMR (400 MHz, DMSO-d₆) δ 10.91 (brs, 1H), 9.75 (brs, 1H), 8.34 (d, 1H, J=4.9 Hz), 7.96 (s, 1H), 7.75 (m, 2H), 7.59 (m, 1H), 7.45 (t, 1H, J=7.9 Hz), 7.37 (d, 1H, J=8.6 Hz), 7.31-7.21 (m, 3H), 7.08 (t, 1H, J=9.3 Hz), 6.59 (d, 1H, J=4.9 Hz), 6.27 (t, 1H, J=5.6 Hz), 4.80 (d, 2H, J=5.5 Hz), 4.09 (t, 2H, J=5.7 Hz), 2.78 (t, 2H, J=5.4 Hz), 2.52 (m, 4H), and 1.68 (m, 4H); MS (ESI): 645.23 [M−H]⁺.

### Example 127

### 2,6-Difluoro-N-[3-(2-(fluoromethyl)-5-{2-[3-fluoro-4-{[2-(1-pyrrolidinyl)ethyl]oxy}phenyl)amino]-4-pyrimidinyl}-1,3-thiazol-4-yl)phenyl]benzamide

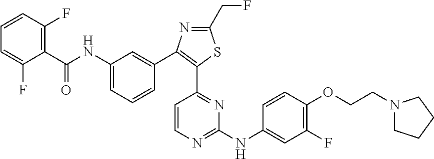

### Step A: 2-Fluoroethanethioamide

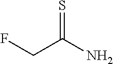

The title compound of Step A was prepared (3.77 g, 54%) from 2-fluoroacetamide (5.78 g, 75.0 mmol) by a procedure analogous to Example 121, Step A, except that the flash column chromatography gradient was run from 20 to 50% EtOAc:hexanes. ¹H NMR (400 MHz, CDCl₃) δ 7.65 (brs, 2H), and 5.12 (d, 2H, J=48.2 Hz).

Step B: N-{3-[5-(2-Chloro-4-pyrimidinyl)-2-(fluoromethyl)-1,3-thiazol-4-yl]phenyl}-2,6-difluorobenzamide

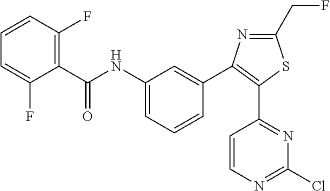

The title compound of Step B was prepared (370 mg, 25%) from N-{3-[(2-chloro-4-pyrimidinyl)acetyl]phenyl}-2,6-difluorobenzamide (1.25 g, 3.22 mmol), prepared by a procedure analogous to Example 1, Step C, and 2-fluoroethanethioamide (450 mg, 4.83 mmol) by a procedure analogous to Example 112, Step A, except that the flash column chromatography gradient was run from 20 to 70% EtOAc:hexanes. ¹H-NMR (400 MHz, CDCl₃) δ 8.42 (d, 1H, J=5.3 Hz), 7.87 (t, 1H, J=1.8 Hz), 7.77 (m, 1H), 7.70 (brs, 1H), 7.50-7.41 (m, 2H), 7.33 (m, 1H), 7.19 (d, 1H, J=5.3 Hz), 7.02 (m, 2H), and 5.67 (d, 2H, J=46.5 Hz); MS (ESI): 460.99 [M+H]⁺.

### Step C: 2,6-Difluoro-N-[3-(2-(fluoromethyl)-5-{2-[(3-fluoro-4-{[2-(1-pyrrolidinyl)ethyl]oxy}phenyl)amino]-4-pyrimidinyl}-1,3-thiazol-4-yl)phenyl]benzamide

The title compound of Example 127 was prepared (65 mg, 37%) from N-{3-[5-(2-chloro-4-pyrimidinyl)-2-(fluoromethyl)-1,3-thiazol-4-yl]phenyl}-2,6-difluorobenzamide (125 mg, 0.271 mmol) and 3-fluoro-4-{[2-(1-pyrrolidinyl)ethyl]oxy}phenyl amine hydrochloride (81 mg, 0.312 mmol), prepared by a procedure analogous to Example 33, Step D, by a procedure analogous to Example 1 Step D. ¹H-NMR (400 MHz, CDCl₃) δ 8.45 (d, 1H, J=4.9 Hz), 7.85-7.75 (m, 3H), 7.59 (d, 1H, J=13.0 Hz), 7.47-7.35 (m, 3H), 7.15 (m, 2H), 7.03-6.94 (m, 3H), 6.67 (d, 1H, J=4.9 Hz), 5.66 (d, 1H, J=46.7 Hz), 4.18 (t, 2H, J=5.5 Hz), 2.93 (t, 2H, J=5.4 Hz), 2.65 (m, 4H), and 1.82 (m, 4H); MS (ESI): 649.20 [M+H]+.

### Example 128

### N-[3-(5-{2-[(4-{[2-(Dimethylamino)ethyl]oxy}-3-fluorophenyl)amino]-4-pyrimidinyl}-2-methyl-1,3-thiazol-4-yl)phenyl]-2,5-difluorobenzamide

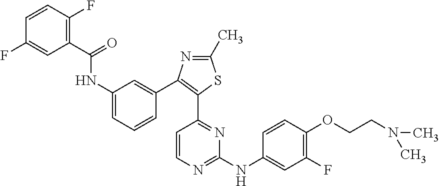

### Step A: N-{3-[5-(2-Chloro-4-pyrimidinyl)-2-methyl-1,3-thiazol-4-yl]phenyl}-2,5-difluorobenzamide

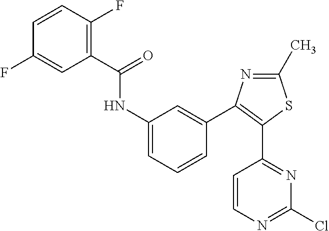

NBS (0.715 g; 4.02 mmol) was added to a stirring suspension of N-{3-[(2-chloro-4-pyrimidinyl)acetyl]phenyl}-2,5-difluorobenzamide (1.52 g; 3.92 mmol), prepared by a procedure analogous to Example 1, Step C, in DCM (20 mL). The resulting clear, homogeneous reaction was stirred for 0.5 h at rt. No evidence of the original starting material was found by LC/MS and the reaction was concentrated in vacuo to a crude residue. This crude residue was taken up in DMF (25 mL) and thioacetamide (0.3 g; 3.99 mmol) in DMF (5 mL) was added. The reaction was heated at 80° C. and the LC/MS at 0.5 h showed the absence of the starting intermediate. The reaction was concentrated in vacuo to a crude residue. Trituration of the crude residue gave the desired product (0.65 g, 37%). ¹H-NMR (400 mHz, CDCl₃-d) δ 8.54 (d, 1H, J=15.9 Hz), 8.36 (d, 1H, J=5.3 Hz), 7.9-7.8 (m, 3H), 7.48 (t, 1H, J=7.9 Hz), 7.33 (d, 1H, J=7.7 Hz), 7.24-7.16 (m, 2H), 7.12 (d, 1H, J=5.3 Hz), and 2.82 (s, 3H); MS

### Step B: N-[3-(5-{2-[(4-{[2-(Dimethylamino)ethyl]oxy}-3-fluorophenyl)amino]-4-pyrimidinyl}-2-methyl-1,3-thiazol-4-yl)phenyl]-2,5-difluorobenzamide

The preparation was carried out in a manner similar to Example 1, Step D using N-{3-[5-(2-chloro-4-pyrimidinyl)-2-methyl-1,3-thiazol-4-yl]phenyl}-2,5-difluorobenzamide (0.1 g, 0.23 mmol) with {2-[(4-amino-2-fluorophenyl)oxy]ethyl}dimethylamine hydrochloride (0.052 g, 0.22 mmol), prepared by a procedure analogous to Example 105, Step B. Purification using RP HPLC gave the desired product (0.048 g, 35%). ¹H-NMR (400 mHz, CDCl₃-d) δ 8.50 (d, 1H, J=16.3 Hz), 8.17 (d, 1H, J=5.3 Hz), 7.86-7.82 (m, 2H), 7.79 (brs, 1H), 7.67 (brs, 1H), 7.62 (dd, 1H, J=13.4 Hz and J=2.4 Hz), 7.43 (t, 2H, J=7.9 Hz), 7.33 (d, 1H, 7.7 Hz), 7.22-7.12 (m, 2H) 7.10 (d, 1H, J=8.6 Hz), 7.06 (s, 1H), 6.95 (t, 1H, J=9.0 Hz), 6.60 (d, 1H, J=5.1 Hz), 4.11 (t, 2H, J=5.9 Hz), 2.77 (s, 3H), 2.74 (t, 2H, J=5.8 Hz), and 2.34 (s, 6H); MS (ESI) 605 [M+H] 603 [M−H].

### Example 129

### N-[4-Chloro-3-(trifluoromethyl)phenyl]-N′-[3-(2-methyl-5-{2-[2-methyl-1,2,3,4-tetrahydro-7-isoquinolinyl)amino]-4-pyrimidinyl}-1,3-thiazol-4-yl)phenyl]urea

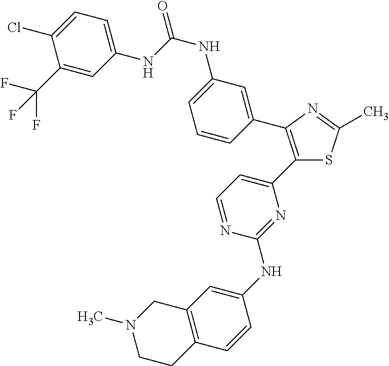

### Step A: Ethyl 3-{[(2-propen-1-yloxy)carbonyl]amino}benzoate

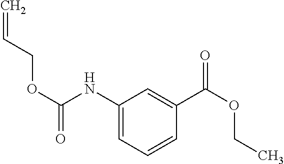

A solution of ethyl-3-aminobenzoate (25.0 g, 151.33 mmol) in DCM (500 mL) was cooled to 0° C. 2,6-Lutidine (19.46 g, 181.60 mmol) was added to the solution followed by addition of allyl chloroformate (20.07 g, 166.46 mmol). Following addition, the reaction was removed from ice bath and stirred at rt for 30 min. The reaction was quenched with saturated NaHCO₃ and the layers were separated. The mixture was extracted with DCM x3, and the combined organics were washed with 10% HCl/H₂O x3, dried over MgSO₄ and the solvent was removed to give ethyl 3-{[(2-propen-1-yloxy)carbonyl]amino}benzoate (38.80 g, 80% yield). ¹H-NMR (400 MHz, DMSO-d₆) δ 9.96 (s, 1H), 8.15 (s, 1H), 7.66-7.72 (m, 1H), 7.59 (d, J=7.7 Hz, 1H), 7.43 (t, J=7.9 Hz, 1H), 5.94-6.04 (m, 1H), 5.37 (dd, J=17.4 and 1.7 Hz, 1H), 5.24 (dd, J=10.6 and 1.5 Hz, 1H), 4.63 (d, J=5.5 Hz, 2H), 4.31 (q, J=7.3 Hz, 2H), and 1.31 (t, J=7.1 Hz, 3H); ES-LCMS m/z 250 (M+H).

### Step B: 2-Propen-1-yl{3-[(2-chloro-4-pyrimidinyl)acetyl]phenyl}carbamate

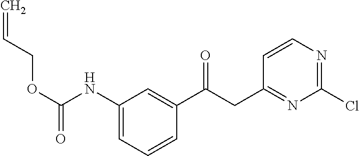

Ethyl 3-{[(2-propen-1-yloxy)carbonyl]amino}benzoate (20.0 g, 80.24 mmol) was dissolved in 1 M LiHMDS in THF (260 mL) and cooled to 0° C. A solution containing 2-chloro-4-methylpyrimidine (10.32 g, 80.24 mmol) in 20 mL dry THF was added to the reaction mixture. The reaction was stirred at 0° C. for 2 h, quenched with MeOH (100 mL), dried directly onto silica, and purified via flash chromatography EtOAc/CH₂Cl₂ 0-100% gradient run over 60 min. The desired fractions were combined and the solvent was removed to give 2-propen-1-yl{3-[(2-chloro-4-pyrimidinyl)acetyl]phenyl}carbamate (13.6 g, 51% yield).ES-LCMS m/z 332 (M+H).

### Step C: 2-Propen-1-yl{3-[5-(2-chloro-4-pyrimidinyl)-2-methyl-1,3-thiazol-4-yl]phenyl}carbamate

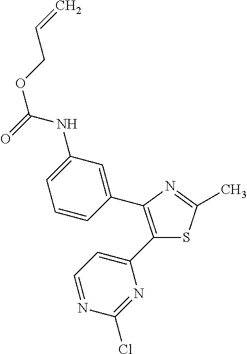

To a solution of 2-propen-1-yl{3-[(2-chloro-4-pyrimidinyl)acetyl]phenyl}carbamate (3.20 g, 9.67 mmol) in DMF (75 mL), NBS (1.72 g, 9.67 mmol) was added while stirring at rt for 1 hour. Thioacetamide (0.87 g, 11.60 mmol) was added to the reaction mixture and allowed to stir at rt overnight. The reaction was diluted with EtOAc (250 mL) and water (250 mL) and extracted with EtOAc x3. The organics were combined, washed with brine x3, dried over MgSO₄, loaded onto silica, and purified via flash chromatography EtOAc/Hexanes 0-100% gradient run over 30 min. The desired fractions were combined to give 2-propen-1-yl{3-[5-(2-chloro-4-pyrimidinyl)-2-methyl-1,3-thiazol-4-yl]phenyl}carbamate (2.40 g, 64% yield). ES-LCMS m/z 387 (M+H).

### Step D: 2-Propen-1-yl[3-(2-methyl-5-{2-[(2-methyl-1,2,3,4-tetrahydro-7-isoquinolinyl)amino]-4-pyrimidinyl}-1,3-thiazol-4-yl)phenyl]carbamate

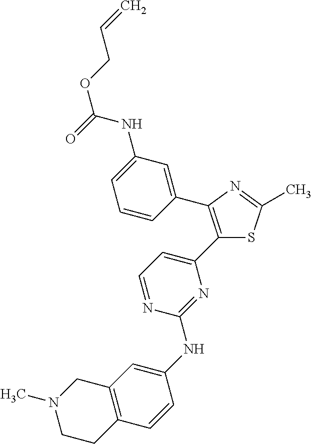

Title compound of Step D was synthesized using standard microwave chloride displacement conditions analogous to Example 1, Step D, using 2-propen-1-yl{3-[5-(2-chloro-4-pyrimidinyl)-2-methyl-1,3-thiazol-4-yl]phenyl}carbamate (1.00 g, 2.58 mmol) and 2-methyl-1,2,3,4-tetrahydro-7-isoquinolinamine (0.46 g, 2.84 mmol) and was purified via flash chromatography MeOH/CH₂Cl₂ 0-20% gradient run over 30 min. The desired fractions were combined to give 2-propen-1-yl[3-(2-methyl-5-{2-[(2-methyl-1,2,3,4-tetrahydro-7-isoquinolinyl)amino]-4-pyrimidinyl}-1,3-thiazol-4-yl)phenyl]carbamate (0.99 g, 74% yield). ES-LCMS m/z 513 (M+H).

### Step E: N-{4-[4-(3-Aminophenyl)-2-methyl-1,3-thiazol-5-yl]-2-pyrimidinyl}-2-methyl-1,2,3,4-tetrahydro-7-isoquinolinamine

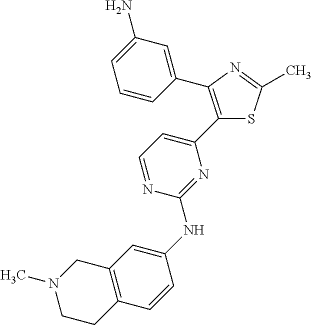

2-Propen-1-yl[3-(2-methyl-5-{2-[(2-methyl-1,2,3,4-tetrahydro-7-isoquinolinyl)amino]-4-pyrimidinyl}-1,3-thiazol-4-yl)phenyl]carbamate (1.52 g, 2.97 mmol) was dissolved in DCM (10 mL) and tributyltinhydride (0.86 g, 2.97 mmol) was added, followed by palladium tetrakis triphenylphosine (0.171 g, 0.15 mmol) and H₂O (0.5 mL). The reaction was stirred at it for 30 min, loaded directly onto silica, and purified via flash chromatography EtOAc/MeOH+NH₄OH 0-20% gradient over 30 min. The desired fractions were combined to give N-{4-[4-(3-aminophenyl)-2-methyl-1,3-thiazol-5-yl]-2-pyrimidinyl}-2-methyl-1,2,3,4-tetrahydro-7-isoquinolinamine (0.54 g, 80% yield). ES-LCMS m/z 429 (M+H).

### Step F: N-[4-chloro-3-(trifluoromethyl)phenyl]-N′-[3-(2-methyl-5-{2-[(2-methyl-1,2,3,4-tetrahydro-7-isoquinolinyl)amino]-4-pyrimidinyl}-1,3-thiazol-4-yl)phenyl]urea

To a solution containing N-{4-[4-(3-aminophenyl)-2-methyl-1,3-thiazol-5-yl]-2-pyrimidinyl}-2-methyl-1,2,3,4-tetrahydro-7-isoquinolinamine (0.090 g, 0.19 mmol) and 1-chloro-4-isocyanato-2-(trifluoromethyl)benzene (0.050 g, 0.23 mmol) in DMF (2 mL) was added HATU (0.087 g, 0.23 mmol), and diisopropylethylamine (0.074 g, 0.57 mmol). The reaction was stirred at rt for 30 min, diluted with EtOAc (10 mL), washed with brine x3, and dried over MgSO₄. Purification by HPLC gave N-[4-chloro-3-(trifluoromethyl)phenyl]N′-[3-(2-methyl-5-{2-[(2-methyl-1,2,3,4-tetrahydro-7-isoquinolinyl)amino]-4-pyrimidinyl}-1,3-thiazol-4-yl)phenyl]urea (0.045 g, 36% yield). ¹H-NMR (400 MHz, DMSO-d₆) δ 8.23 (d, J=5.1 Hz, 1H), 8.10 (s, 1H), 7.78 (s, 1H), 7.59-7.69 (m, 3H), 7.39-7.47 (m, 3H), 7.24 (t, J=8.5 Hz, 1H), 6.99 (d, J=9.2 Hz, 2H), 6.43 (d, J=5.1 Hz, 1H), 3.42 (s, 3H), 2.64-2.76 (m, 6H), 2.55 (t, J=6.0 Hz, 2H), and 2.32 (s, 3H); ES-LCMS m/z 650 (M+H).

### Example 130

### N-[3-(2-Methyl-5-{2-[(2-methyl-1,2,3,4-tetrahydro-7-isoquinolinyl)amino]-4-pyrimidinyl}-1,3-thiazol-4-yl)phenyl]-2-(methyloxy)benzamide

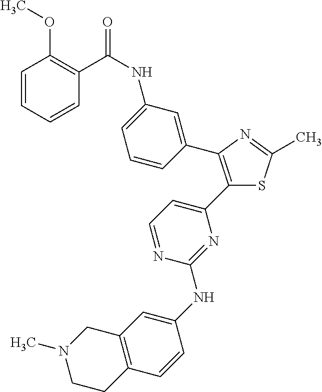

Title compound of Example 130 was synthesized by the standard acylating procedure analogous to Example 129, Step F, combining N-{4-[4-(3-aminophenyl)-2-methyl-1,3-thiazol-5-yl]-2-pyrimidinyl}-2-methyl-1,2,3,4-tetrahydro-7-isoquinolinamine (0.090 g, 0.19 mmol), prepared by a procedure analogous to Example 129, Step E, and 2-(methyloxy)benzoyl chloride (0.039 g, 0.23 mmol) and was purified by HPLC and free based to give N-[3-(2-methyl-5-{2-[(2-methyl-1,2,3,4-tetrahydro-7-isoquinolinyl)amino]-4-pyrimidinyl}-1,3-thiazol-4-yl)phenyl]-2-(methyloxy)benzamide (0.056 g, 52% yield). ¹H-NMR (400 MHz, DMSO-d₆) δ 10.23 (s, 1H), 9.59 (s, 1H), 8.30 (d, J=5.3 Hz, 1H), 8.00 (s, 1H), 7.84 (d, J=8.1 Hz, 1H), 7.60 (d, J=7.67 Hz, 1H), 7.50 (t, J=7.9 Hz, 1H), 7.40-7.46 (m, 3H), 7.24 (d, J=7.9 Hz, 1H), 7.17 (d, J=8.4 Hz, 1H), 6.99-7.08 (m, 2H), 6.52 (d, J=5.3 Hz, 1H), 3.88 (s, 3H), 3.44 (s, 2H), 2.73-2.76 (m, 5H), 2.57 (t, J=6.0 Hz, 2H), and 2.33 (s, 3H); ES-LCMS m/z 563 (M+H).

### Example 131

### N-[3-(2-Methyl-5-{2-[(2-methyl-1,2,3,4-tetrahydro-7-isoquinolinyl)amino]-4-pyrimidinyl}-1,3-thiazol-4-yl)phenyl]-3-(trifluoromethyl)benzamide

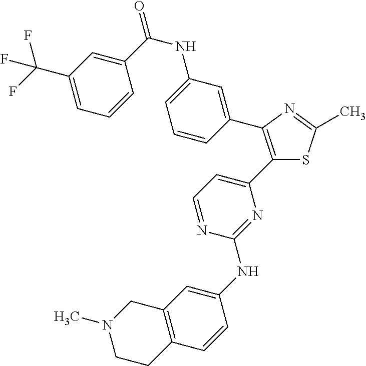

Title compound of Example 131 was synthesized by a standard acylating procedure analogous to Example 129, Step F, combining N-{4-[4-(3-aminophenyl)-2-methyl-1,3-thiazol-5-yl]-2-pyrimidinyl}-2-methyl-1,2,3,4-tetrahydro-7-isoquinolinamine (0.090 g, 0.19 mmol), prepared by a procedure analogous to Example 129, Step E, and 3-(trifluoromethyl)benzoyl chloride (0.048 g, 0.23 mmol) and was purified by HPLC and free based to give N-[3-(2-methyl-5-{2-[(2-methyl-1,2,3,4-tetrahydro-7-isoquinolinyl)amino]-4-pyrimidinyl}-1,3-thiazol-4-yl)phenyl]-3-(trifluoromethyl)benzamide (0.057 g, 50% yield). ¹H-NMR (400 MHz, DMSO-d₆) δ 10.57-10.63 (m, 1H), 9.60 (s, 1H), 8.25-8.33 (m, 3H), 8.05 (s, 1H), 7.95 (dd, J=14.4 and 8.3 Hz, 2H), 7.79 (t, J=7.6 Hz, 1H), 7.42-7.48 (m, 3H), 7.28 (d, J=7.7 Hz, 1H), 7.00 (d, J=9.2 Hz, 1H), 6.55 (d, J=4.9 Hz, 1H), 3.43 (s, 2H), 2.72-2.78 (m, 5H), 2.57 (t, J=6.6 Hz, 2H), and 2.33 (s, 3H); ES-LCMS m/z 601 (M+H).

### Example 132

### N-[3-(2-Methyl-5-{2-[(2-methyl-1,2,3,4-tetrahydro-7-isoquinolinyl)amino]-4-pyrimidinyl}-1,3-thiazol-4-yl)phenyl]-3-(methyloxy)benzamide trifluoroacetate

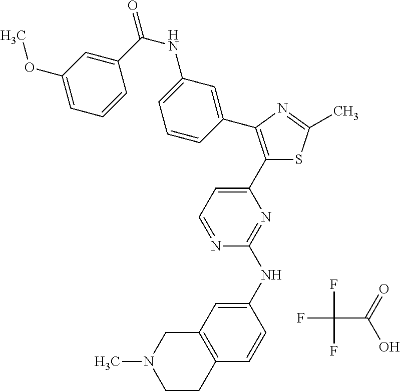

Title compound of Example 132 was synthesized by a standard acylating procedure analogous to Example 129, Step F, combining N-{4-[4-(3-aminophenyl)-2-methyl-1,3-thiazol-5-yl]-2-pyrimidinyl}-2-methyl-1,2,3,4-tetrahydro-7-isoquinolinamine (0.090 g, 0.19 mmol), prepared by a procedure analogous to Example 129, Step E, and 3-(methyloxy)benzoyl chloride (0.039 g, 0.23 mmol) and was purified by HPLC to give N-[3-(2-methyl-5-{2-[(2-methyl-1,2,3,4-tetrahydro-7-isoquinolinyl)amino]-4-pyrimidinyl}-1,3-thiazol-4-yl)phenyl]-3-(methyloxy)benzamide trifluoroacetate (0.061 g, 57% yield). ¹H-NMR (400 MHz, DMSO-d₆) δ 10.32 (d, J=0.92 Hz, 1H), 9.79 (d, J=8.4 Hz, 1H), 8.33 (d, J=5.7 Hz, 1H), 8.06 (t, J=2.6 Hz, 1H), 7.83-7.93 (m, 1H), 7.57-7.63 (m, 2H), 7.49-7.55 (m, 1H), 7.40-7.48 (m, 3H), 7.22-7.27 (m, 1H), 7.13-7.18 (m, 2H), 6.56-6.61 (m, 1H), 3.78-3.84 (m, 3H), 3.65 (dd, J=10.3 and, 7.1 Hz, 1H), 3.28-3.39 (m, 2H), 2.96-3.06 (m, 2H), 2.91-2.96 (m, 3H), and 2.72-2.76 (m, 3H); ES-LCMS m/z 563 (M+H).

### Example 133

### N-[3-(5-{2-[(3-Chloro-4-{[2-(dimethylamino)ethyl]oxy}Phenyl)amino]-4-pyrimidinyl}-2-methyl-1,3-thiazol-4-yl)phenyl]-N′-[4-chloro-3-(trifluoromethyl)phenyl]urea

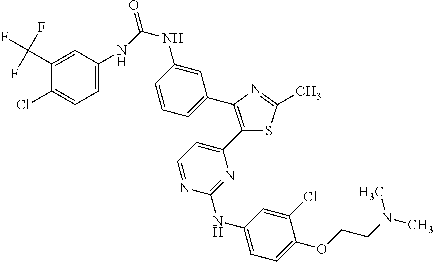

### Step A: N-(3-chloro-4-{[2-(dimethylamino)ethyl]oxy}phenyl)-4-[2-methyl-4-(3-nitrophenyl)-1,3-thiazol-5-yl]-2-pyrimidinamine

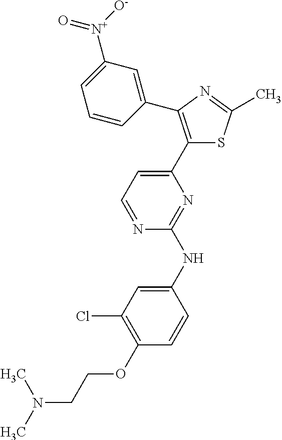

Title compound of Step A was prepared by standard chloride displacement procedure analogous to Example 1, Step D, using 2-chloro-4-[2-methyl-4-(3-nitrophenyl)-1,3-thiazol-5-yl]pyrimidine (3.00 g, 7.97 mmol), prepared in a procedure analogous to Example 103, Step E, and {2-[(4-amino-2-chlorophenyl)oxy]ethyl}dimethylamine hydrochloride (2.85 g, 8.77 mmol) in i-PrOH, and heating to 90° C. overnight, and purifying via flash chromatography 0-20% MeOH+NH₄OH/EtOAc gradient over 30 min. The desired fractions were combined and concentrated to give N-(3-chloro-4-{[2-(dimethylamino)ethyl]oxy}phenyl)-4-[2-methyl-4-(3-nitrophenyl)-1,3-thiazol-5-yl]-2-pyrimidinamine (2.00 g, 49% yield). ES-LCMS m/z 511 (M+H).

### Step B: 4-[4-(3-Aminophenyl)-2-methyl-1,3-thiazol-5-yl]-N-(3-chloro-4-{[2-(dimethylamino)ethyl]oxy}phenyl)-2-pyrimidinamine

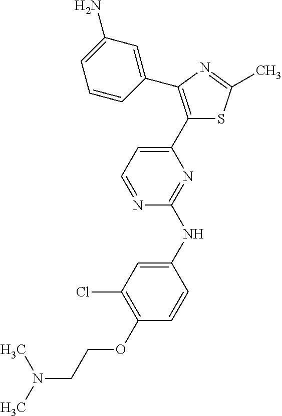

Title compound of Step B was prepared from dissolving N-(3-chloro-4-{[2-(dimethylamino)ethyl]oxy}phenyl)-4-[2-methyl-4-(3-nitrophenyl)-1,3-thiazol-5-yl]-2-pyrimidinamine (2.20 g, 4.31 mmol) in EtOH (100 mL) and adding HOAc (50 mL) and 10% Pd/C (0.300 g). The reaction was stirred at rt on Fischer-Porter device for 2 h under H₂ at 50 psi, filtered through celite plug and concentrated to afford 4-[4-(3-aminophenyl)-2-methyl-1,3-thiazol-5-yl]-N-(3-chloro-4-{[2-(dimethylamino)ethyl]oxy}phenyl)-2-pyrimidinamine (1.91 g, 92% yield). ES-LCMS m/z 481 (M+H).

### Step C: N-[3-(5-{2-[(3-Chloro-4-{[2-(dimethylamino)ethyl]oxy}phenyl)amino]-4-pyrimidinyl}-2-methyl-1,3-thiazol-4-yl)phenyl]-N′-[4-chloro-3-(trifluoromethyl)phenyl]urea

Title compound of Example 133 was synthesized by a standard acylating procedure analogous to Example 129, Step F, combining 4-[4-(3-aminophenyl)-2-methyl-1,3-thiazol-5-yl]-N-(3-chloro-4-{[2-(dimethylamino)ethyl]oxy}phenyl)-2-pyrimidinamine (0.090 g, 0.19 mmol) and 1-chloro-4-isocyanato-2-(trifluoromethyl)benzene (0.050 g, 0.23 mmol), and was purified by HPLC and free based to give N-[3-(5-{2-[(3-chloro-4-{[2-(dimethylamino)ethyl]oxy}phenyl)amino]-4-pyrimidinyl}-2-methyl-1,3-thiazol-4-yl)phenyl]-N′-[4-chloro-3-(trifluoromethyl)phenyl]urea (0.056 g, 42% yield). ¹H-NMR (400 MHz, DMSO-d₆) δ 9.80 (s, 1H), 9.30 (s, 1H), 9.12 (s, 1H), 8.33 (d, J=5.3 Hz, 1H), 8.10 (s, 1H), 7.97 (d, J=2.6 Hz, 1H), 7.76 (s, 1H), 7.59-7.69 (m, 3H), 7.48-7.53 (m, 1H), 7.39 (t, J=7.9 Hz, 1H), 7.14-7.22 (m, 2H), 6.56 (d, J=5.3 Hz, 1H), 4.34-4.39 (m, 2H), 3.56 (d, J=4.56 Hz, 2H), 2.93 (d, J=4.8 Hz, 6H), and 2.74 (s, 3H); ES-LCMS m/z 702 (M+H).

### Example 134

### N-[3-(5-{2-[(3-Chloro-4-{[2-(dimethylamino)ethyl]oxy}phenyl)amino]-4-pyrimidinyl}-2-methyl-1,3-thiazol-4-yl)phenyl]-4-fluorobenzamide

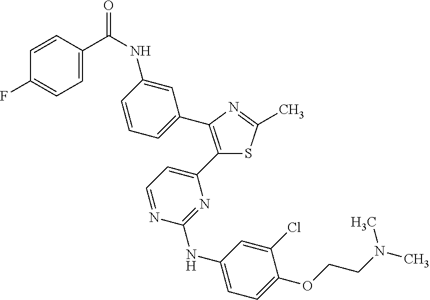

Title compound of Example 134 was synthesized by a standard acylating procedure analogous to Example 129, Step F, combining 4-[4-(3-aminophenyl)-2-methyl-1,3-thiazol-5-yl]-N-(3-chloro-4-{[2-(dimethylamino)ethyl]oxy}phenyl)-2-pyrimidinamine (0.090 g, 0.19 mmol), prepared by a procedure analogous to Example 133, Step B, and 4-fluorobenzoyl chloride (0.036 g, 0.23 mmol) and was purified by HPLC and free based to give N-[3-(5-{2-[(3-chloro-4-{[2-(dimethylamino)ethyl]oxy}phenyl)amino]-4-pyrimidinyl}-2-methyl-1,3-thiazol-4-yl)phenyl]-4-fluorobenzamide (0.057 g, 50% yield). ¹H-NMR (400 MHz, DMSO-d₆) δ 10.38 (s, 1H), 9.80 (s, 1H), 8.34 (d, J=5.1 Hz, 1H), 8.00-8.06 (m, 3H), 7.97 (d, J=2.8 Hz, 1H), 7.89 (dd, J=7.8 and 1.6 Hz, 1H), 7.65 (dd, J=9.1 and 2.7 Hz, 1H), 7.44 (t, J=8.0 Hz, 1H), 7.37 (t, J=9.0 Hz, 2H), 7.27 (d, J=7.7 Hz, 1H), 7.19 (d, J=9.0 Hz, 1H), 6.58 (d, J=5.1 Hz, 1H), 4.34-4.38 (m, 2H), 3.55 (d, J=5.5 Hz, 2H), 2.93 (d, J=4.4 Hz, 6H), and 2.75 (s, 3H); ES-LCMS m/z 603 (M+H).

### Example 135

### N-[3-(5-{2-[(3-Chloro-4-{[2-(dimethylamino)ethyl]oxy}phenyl)amino]-4-pyrimidinyl}-2-methyl-1,3-thiazol-4-yl)phenyl]-3-methylbenzamide

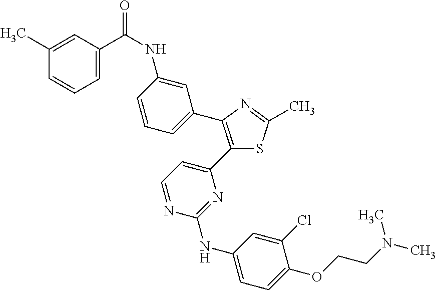

Title compound of Example 135 was synthesized by a standard acylating procedure analogous to Example 129, Step F, combining 4-[4-(3-aminophenyl)-2-methyl-1,3-thiazol-5-yl]-N-(3-chloro-4-{[2-(dimethylamino)ethyl]oxy}phenyl)-2-pyrimidinamine (0.090 g, 0.19 mmol), prepared by a procedure analogous to Example 133, Step B, and 3-methylbenzoyl chloride (0.035 g, 0.23 mmol), and was purified by HPLC and free based to give N-[3-(5-{2-[(3-chloro-4-{[2-(dimethylamino)ethyl]oxy}phenyl)amino]-4-pyrimidinyl}-2-methyl-1,3-thiazol-4-yl)phenyl]-3-methylbenzamide (0.061 g, 54% yield). ¹H-NMR (400 MHz, DMSO-d₆) δ 10.32 (s, 1H), 9.80 (s, 1H), 8.34 (d, J=5.1 Hz, 1H), 8.07 (s, 1H), 7.97 (d, J=2.6 Hz, 1H), 7.90 (d, J=8.2 Hz, 1H), 7.71-7.78 (m, 2H), 7.66 (d, J=9.2 Hz, 1H), 7.38-7.46 (m, 3H), 7.26 (d, J=7.5 Hz, 1H), 7.19 (d, J=9.0 Hz, 1H), 6.58 (d, J=5.3 Hz, 1H), 4.32-4.40 (m, 2H), 3.55 (d, J=4.6 Hz, 2H), 2.93 (d, J=4.4 Hz, 6H), 2.75 (s, 3H), and 2.40 (s, 3H); ES-LCMS m/z 599 (M+H).

### Example 136

### 3-Chloro-N-[3-(5-{2-[(3-chloro-4-{[2-(dimethylamino)ethyl]oxy}phenyl)amino]-4-pyrimidinyl}-2-methyl-1,3-thiazol-4-yl)phenyl]benzamide

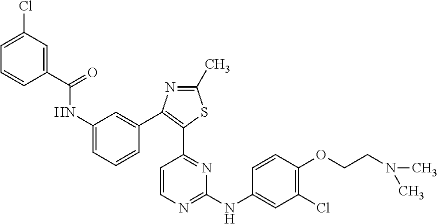

Title compound of Example 136 was synthesized by a standard acylating procedure analogous to Example 129, Step F, combining 4-[4-(3-aminophenyl)-2-methyl-1,3-thiazol-5-yl]-N-(3-chloro-4-{[2-(dimethylamino)ethyl]oxy}phenyl)-2-pyrimidinamine (0.090 g, 0.19 mmol), prepared by a procedure analogous to Example 133, Step B, and 3-chlorobenzoyl chloride (0.035 g, 0.23 mmol), and was purified by HPLC and free based to give 3-chloro-N-[3-(5-{2-[(3-chloro-4-{[2-(dimethylamino)ethyl]oxy}phenyl)amino]-4-pyrimidinyl}-2-methyl-1,3-thiazol-4-yl)phenyl]benzamide (0.068 g, 58% yield). ¹H-NMR (400 MHz, DMSO-d₆) δ 10.64 (s, 1H), 9.81 (s, 1H), 8.40 (d, J=1.8 Hz, 1H), 8.34 (d, J=5.1 Hz, 1H), 8.26 (dd, J=8.3 and 1.9 Hz, 1H), 8.04 (s, 1H), 7.88-7.99 (m, 3H) 7.66 (dd, J=9.0 and 2.6 Hz, 1H), 7.47 (t, J=8.1 Hz, 1H), 7.31 (d, J=7.7 Hz, 1H), 7.20 (d, J=9.2 Hz, 1H), 6.59 (d, J=5.1 Hz, 1H), 4.32-4.41 (m, 2H), 3.56 (q, J=4.8 Hz, 2H), 2.93 (d, J=4.6 Hz, 6H), and 2.75 (s, 3H); ES-LCMS m/z 619 (M+H).

### Example 137

### 2-Fluoro-N-[3-(2-methyl-5-{2-[(2-methyl-1,2,3,4-tetrahydro-7-isoquinolinyl)amino]-4-pyrimidinyl}-1,3-thiazol-4-yl)phenyl]-3-pyridinecarboxamide trifluoroacetate

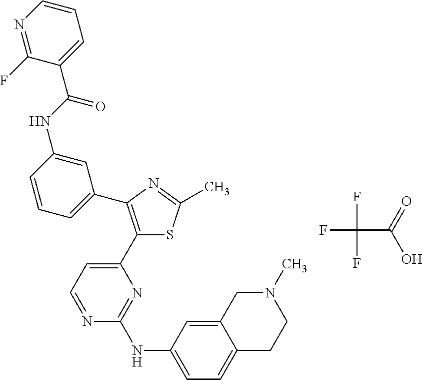

N-{4-[4(3-Aminophenyl)-2-methyl-1,3-thiazol-5-yl]-2-pyrimidinyl}-2-methyl-1,2,3,4-tetrahydro-7-isoquinolinamine (0.082 g, 0.19 mmol), prepared by a procedure analogous to Example 129, Step E, was combined with HATU (0.087 g, 0.23 mmol), 2-fluoro-3-pyridinecarboxylic acid (0.032 g, 0.23 mmol) and diisopropylethylamine (0.074 g, 0.57 mmol) in DMF (2 mL) and stirred at rt for 30 min. The reaction was diluted with EtOAc (10 mL), washed with brine x3, dried over MgSO₄, and purified via HPLC to give 2-fluoro-N-[3-(2-methyl-5-{2-[(2-methyl-1,2,3,4-tetrahydro-7-isoquinolinyl)amino]-4-pyrimidinyl}-1,3-thiazol-4-yl)phenyl]-3-pyridinecarboxamide trifluoroacetate (0.054 g, 51% yield). ¹H-NMR (400 MHz, DMSO-d₆) δ 10.70 (s, 1H), 9.82 (s, 1H), 8.41 (d, J=4.9 Hz, 1H), 8.35 (d, J=5.3 Hz, 1H), 8.25 (ddd, J=9.7, 7.7, and 2.3 Hz, 1H), 8.00 (d, J=1.7 Hz, 1H), 7.78 (dd, J=7.4 and 1.7 Hz, 1H), 7.57-7.62 (m, 2H), 7.52 (ddd, J=7.3, 5.0, and 1.8 Hz, 1H), 7.46 (t, J=7.8 Hz, 1H), 7.30 (dt, J=7.6 and 1.1 Hz, 1H), 7.17 (d. J=8.4 Hz, 1H), 6.59 (d, J=5.1 Hz, 1H), 3.62-3.71 (m, 2H), 3.27-3.39 (m, 2H), 2.99-3.08 (m, 2H), 2.95 (d, J=4.6 Hz, 3H), and 2.75 (s, 3H); ES-LCMS m/z 552 (M+H).

### Example 138

### N-[3-(5-{2-[(3-Fluorophenyl)amino]-4-pyrimidinyl}-2-methyl-1,3-thiazol-4-yl)phenyl]-2-(2-thienyl)acetamide

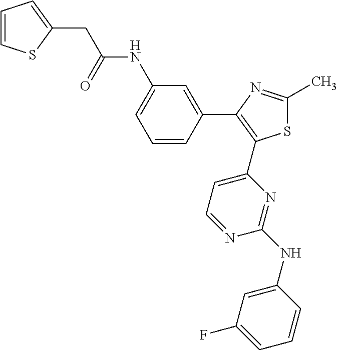

### Step A: 4-[2-Bromo-4-(3-nitrophenyl)-1,3-thiazol-5-yl]-2-chloropyrimidine

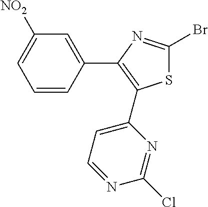

To obtain the title compound of Step A, 5-(2-chloro-4-pyrimidinyl)-4-(3-nitrophenyl)-1,3-thiazol-2-amine (0.9 g, 2.7 mmol), prepared by a procedure analogous to Example 64, Step B, tert-butyl nitrite (0.5 ml, 4 mmol) and Cu(II)Br (0.96 g, 4.3 mmol) were reacted using a procedure analogous to Example 101, Step A. Yield 770 mg (72%). ¹H-NMR (400 MHz, DMSO-d₆) δ 8.64 (d, J=5.2 Hz, 1H), 8.43 (s, 1H), 8.36 (dd, J=8.0, and 2.5 Hz, 1H), 8.04 (d, J=8.0 Hz, 1H), 7.76 (t, J=8.1 Hz, 1H), and 7.26 (d, J=5.4 Hz, 1H).

### Step B: 2-Chloro-4-[2-methyl-4-(3-nitrophenyl)-1,3-thiazol-5-yl]pyrimidine

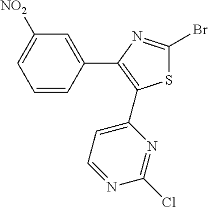

The title compound of Step B was prepared from 4-[2-bromo-4-(3-nitrophenyl)-1,3-thiazol-5-yl]-2-chloropyrimidine (0.77 g, 1.9 mmol), 2 M (CH₃)₂Zn in toluene (1 mL, 0.21 mmol) and palladium tetrakis triphenylphosphine (0.14 g, 0.012 mmol) by a method analogous to Example 101, Step B. Yield 300 mg (47%). ¹H-NMR (300 MHz, DMSO-d₆) δ 8.60 (d, J=5.3 Hz, 1H), 8.41 (s, 1H), 8.32 (dd, J=8.1, and 1.8 Hz, 1H), 8.02 (d, J=7.5 Hz, 1H), 7.73 (t, J=8.0 Hz, 1H), 7.27 (d, J=5.5 Hz, 1H), and 2.77 (s, 3H); MS (ESI) m/z 333 (M+H)⁺.

### Step C: 3-[5-(2-Chloro-4-pyrimidinyl)-2-methyl-1,3-thiazol-4-yl]phenyl amine

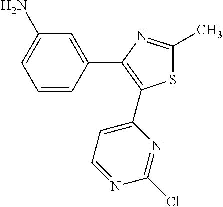

To obtain the desired compound, 2-chloro-4-[2-methyl-4-(3-nitrophenyl)-1,3-thiazol-5-yl]pyrimidine (0.3 g, 0.9 mmol) was dissolved in EtOH (4 mL). Pt/C (150 mg, 5% by wt) was added and the resulting mixture was placed under H₂ (50 psi) for 12 hours. The reaction was then filtered through celite and concentrated to yield 200 mg (74%) of the title compound of Step C: MS (ESI) m/z 303 (M+H)⁺.

### Step D: N-{3-[5-(2-Chloro-4-pyrimidinyl)-2-methyl-1,3-thiazol-4-yl]phenyl}-2-(2-thienyl)acetamide

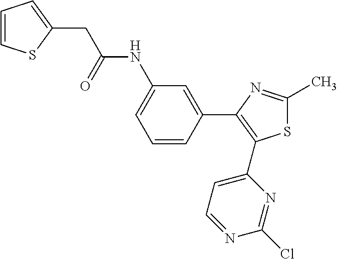

To obtain the title compound of Step D, 3-[5-(2-chloro-4-pyrimidinyl)-2-methyl-1,3-thiazol-4-yl]phenyl amine (0.2 g, 0.66 mmol) was dissolved in DCM (6 mL) and placed in a 0° C. ice bath. 2-Thienylacetyl chloride (0.12 mL, 0.73 mmol) was added and the reaction was then stirred 1 h at 0° C. The reaction was quenched by the addition of saturated NaHCO₃ and extracted with DCM to give an oil which was adhered to silica gel and chromatographed with EtOAc and hexanes to yield 85 mg (30%) of the title compound of Step D. MS (ESI) m/z 427 (M+H)⁺.

### Step E: N-[3-(5-{2-[(3-Fluorophenyl)amino]-4-pyrimidinyl}-2-methyl-1,3-thiazol-4-yl)phenyl]-2-(2-thienyl)acetamide

The title compound of Example 138 was prepared from N-{3-[5-(2-chloro-4-pyrimidinyl)-2-methyl-1,3-thiazol-4-yl]phenyl}-2-(2-thienyl)acetamide (0.85 g, 0.2 mmol) and 3-fluorophenyl amine (0.22 g, 0.2 mmol) by a procedure analogous to Example 1, Step D. Yield 41 mg (41%). ¹H-NMR (300 MHz, DMSO-d₆) δ 10.32 (s, 1H), 9.96 (s, 1H), 8.35 (d, J=5.3 Hz, 1H), 7.83 (s, 1H), 7.76 (d, J=12.5 Hz, 1H), 7.69 (d, J=8.1 Hz, 1H), 7.47 (d, J=8.1 Hz, 1H), 7.42-7.35 (m, 2H), 7.28 (m, 1H), 7.21 (d, J=7.7 Hz, 1H), 6.98-6.94 (m, 2H), 6.75 (t, J=8.6 Hz, 1H), 6.57 (d, J=5.2 Hz, 1H), 3.86 (s, 2H), and 2.73 (s, 3H); HRMS C₂₆H₂₁N₅OFS₂ (M+H)⁺ calcd 502.1172. found 502.1164.

### Example 139

### N-{3-[5-{2-[(3-Chloro-4-{[2-(dimethylamino)ethyl]oxy}phenyl)amino]-4-pyrimidinyl}-2-(4-hydroxyphenyl)-1,3-thiazol-4-yl]phenyl}-2,6-difluorobenzamide

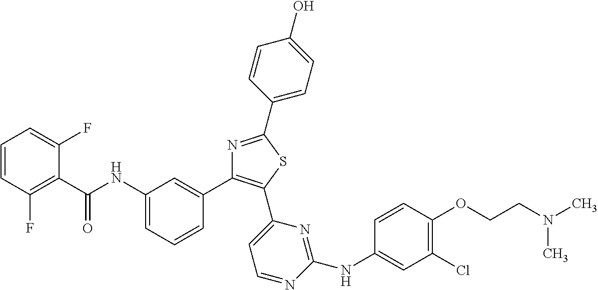

### Step A: N-{3-[5-(2-Chloro-4-pyrimidinyl)-2-(4-hydroxyphenyl)-1,3-thiazol-4-yl]phenyl}-2,6-difluorobenzamide

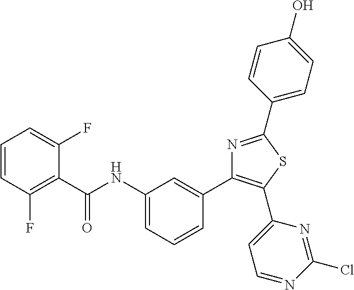

To a solution containing 100 mg (0.197 mmol) of N-{3-[2-bromo-5-(2-chloro-4-pyrimidinyl)-1,3-thiazol-4-yl]phenyl}-2,6-difluorobenzamide, prepared by a procedure analogous to Example 101, Step A, 2 mL of DMF, and 0.25 mL of H₂O was added 29 mg (0.207 mmol) of (4-hydroxyphenyl)boronic acid, 13 mg (0.019 mmol) of dichlorobis(triphenylphosphine) palladium (II), and 73 mg (0.591 mmol) of Na₂CO₃. The reaction mixture was heated at 70° C. for 13 h, then taken up in EtOAc, washed with H₂O, and dried over MgSO₄. The residue was subjected to silica gel chromatography to give 25 mg (25%) of the title compound of Step A: ¹H-NMR (400 MHz, DMSO-D6) δ 10.96 (s, 1H), 10.25 (s, 1H), 8.58 (d, J=5.1 Hz, 1H), 7.98 (s, 1H), 7.92 (d, J=8.6 Hz, 2H), 7.84 (d, J=7.3 Hz, 1H), 7.56-7.63 (m, 1H), 7.50 (t, J=7.9 Hz, 1H), 7.40 (d, J=7.7 Hz, 1H), 7.20-7.28 (m, 3H), and 6.90 (d, J=8.6 Hz, 2H); ESIMS: 521.08 (M+H⁺).

### Step B: N-{3-[5-{2-[(3-Chloro-4-{[2-(dimethylamino)ethyl]oxy}phenyl)amino]-4-pyrimidinyl}-2-(4-hydroxyphenyl)-1,3-thiazol-4-yl]phenyl}-2,6-difluorobenzamide

To a solution containing 25 mg (0.048 mmol) of N-{3-[5-(2-chloro-4-pyrimidinyl)-2-(4-hydroxyphenyl)-1,3-thiazol-4-yl]phenyl}-2,6-difluorobenzamide and 2 mL of i-PrOH was added 13 mg (0.053 mmol) 3-chloro-4-{[2-(dimethylamino)ethyl]oxy}phenyl amine and 0.1 mL of a 4.0 M solution of HCl in dioxane. The reaction mixture was heated at 90° C. for 13 h, then allowed to cool and quenched by the addition of TEA. The solvents were removed and the residue was subjected to silica gel chromatography to give 10 mg (29%) of the title compound of Example 172 as a yellow solid: ¹H-NMR (400 MHz, MeOH-D4) δ 8.13 (d, J=5.13 Hz, 1H), 7.94-7.96 (m, 1H), 7.81-7.85 (m, 3H), 7.49-7.52 (m, 2H), 7.36-7.41 (m, 2H), 7.08 (t, J=8.1 Hz, 2H), 6.99 (d, J=7.0 Hz, 1H), 6.85-6.88 (m, 3H), 6.75 (d, J=2.8 Hz, 1H), 6.59 (dd, J=8.6 and 2.8 Hz, 1H), 6.51 (d, J=5.3 Hz, 1H), 4.16 (t, J=5.2 Hz, 2H), 4.06-4.08 (m, 3H), and 2.94 (t, J=5.2 Hz, 6H); HRMS calcd for C₃₆H₃₀ClF₂N₆O₃S: 699.1751. Found: 699.1751.

### Example 140

### N-{3-[5-{2-[(3-Chloro-4-{[2-(dimethylamino)ethyl]oxy}phenyl)amino]-4-pyrimidinyl}-2-(4-hydroxyphenyl)-1,3-thiazol-4-yl]phenyl}-2,5-difluorobenzamide

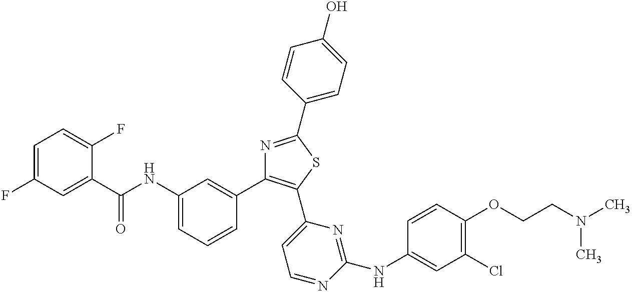

### Step A: N-{3-[2-Bromo-5-(2-Chloro-4-pyrimidinyl)-1,3-thiazol-4-yl]phenyl}-2,5-difluorobenzamide

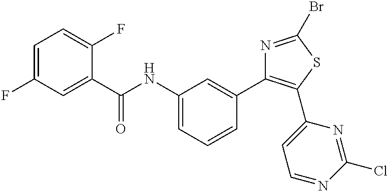

To a solution containing 1.0 g (2.25 mmol) of N-[3-(2-amino-5-{2-[(3-chloro-4-{[2-(dimethylamino)ethyl]oxy}phenyl)amino]-4-pyrimidinyl}-1,3-thiazol-4-yl)phenyl]-2,5-difluorobenzamide, prepared by a procedure analogous to Example 58, Step A, and 20 mL of acetonitrile was added 0.56 mL (4.75 mmol) of/ort-butyl nitrite and 0.50 g (2.28 mmol) of copper (II) bromide. The reaction mixture was allowed to stir for 13 h and quenched by the addition of H₂O. The precipitate was filtered to give 0.99 g (86%) of the title compound of Step A as an off white solid. ¹H-NMR (400 MHz, DMSO-d₆) δ 10.65 (s, 1H), 8.66 (dd, J=5.3 and 0.9 Hz, 1H), 7.98 (s, 1H), 7.85 (d, J=7.1 Hz, 1H), 7.51-7.56 (m, 1H), 7.43-7.49 (m, 3H), 7.36 (d, J=8.4 Hz, 1H), and 7.25 (d, J=6.0 Hz, 1H); ESIMS: 508.98 (M+H⁺).

### Step B: N-{3-[5-(2-Chloro-4-pyrimidinyl)-2-(4-hydroxyphenyl)-1,3-thiazol-4-yl]phenyl}-2,5-difluorobenzamide

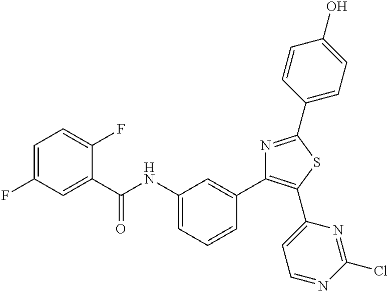

To a solution containing 0.99 g (1.95 mmol) of N-{3-[2-bromo-5-(2-chloro-4-pyrimidinyl)-1,3-thiazol-4-yl]phenyl}-2,5-difluorobenzamide and 20 mL of dioxane was added 0.54 g (3.90 mmol) of (4-hydroxyphenyl)boronic acid, 136 mg (0.195 mmol) of dichlorobis(triphenylphosphine) palladium (II), and 1.25 g (5.85 mmol) of potassium phosphate. The reaction mixture was heated at 70° C. for 13 h and the solvents were removed under reduced pressure. The residue was subjected to silica gel chromatography to give 0.55 g (55%) of the title compound of Step B: ¹H-NMR (400 MHz, DMSO-d₆) δ 10.67 (s, 1H), 10.26 (s, 1H), 8.59 (d, J=5.5 Hz, 1H), 8.01 (s, 1H), 7.87-7.95 (m, 3H), 7.49-7.58 (m, 2H), 7.38-7.47 (m, 3H), 7.19 (d, J=5.3 Hz, 1H), and 6.91 (d, J=8.8 Hz, 2H); ESIMS: 521.14 (M+H⁺).

### Step C: N-{3-[5-{2-[(3-Chloro-4-{[2-(dimethylamino)ethyl]oxy}phenyl)amino]-4-pyrimidinyl}-2-(4-hydroxyphenyl)-1,3-thiazol-4-yl]phenyl}-2,5-difluorobenzamide

To a slurry containing 114 mg (0.219 mmol) of N-{3-[5-(2-chloro-4-pyrimidinyl)-2-(4-hydroxyphenyl)-1,3-thiazol-4-yl]phenyl}-2,5-difluorobenzamide and 2 mL of 2-propanol was added 55 mg (0.053 mmol) of 3-chloro-4-{[2-(dimethylamino)ethyl]oxy}phenyl amine and 0.1 mL of a 4.0M solution of HCl in dioxane. The reaction mixture was heated at 90° C. for 48 h, then allowed to cool and quenched by the addition of TEA. The solvents were removed and the residue was subjected to silica gel chromatography to give 36 mg (24%) of the title compound of Example 173 as a yellow solid: ¹H-NMR (400 MHz, DMSO-d₆) δ 10.66 (s, 1H), 10.20 (s, 1H), 9.75 (s, 1H), 8.34 (d, J=5.3 Hz, 1H), 7.99-8.04 (m, 2H), 7.85-7.88 (m, 3H), 7.54-7.62 (m, 2H), 7.42-7.52 (m, 3H), 7.37 (d, J=7.9 Hz, 1H), 7.13 (d, J=9.3 Hz, 1H), 6.92 (d, J=8.8 Hz, 2H), 6.57 (d, J=4.9 Hz, 1H), 4.10 (t, J=5.8 Hz, 2 H), 2.63-2.70 (m, 2H), 2.26 (s, 6H), and; HRMS calcd for C₃₆H₃₀CIF₂N₆O₃S (M+H⁺): 699.1751. Found: 699.1751.

### Example 141

### 2,6-Difluoro-N-[3-(5-{2-[(3-fluorophenyl)amino]-4-pyrimidinyl}-1,3-oxazol-4-yl)phenyl]benzamide

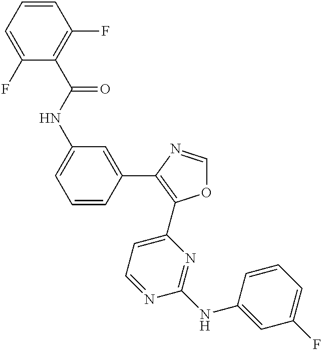

### Step A: 4-Methyl-2-(methylthio)pyrimidine

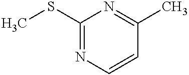

The starting 4-methyl-2(1H)-pyrimidinethione (32.5 g, 0.2 mol) was dissolved in a mixture of EtOH (300 mL), NaOH (15 g) and H₂O (370 mL). To this solution, methyliodide (26 g, 0.18 mol) was added. After 2 h, the reaction was complete. Then it was evaporated to remove EtOH. The reaction mixture was extracted with Et₂O (400 mL, 200 mL, 200 mL). The total organic phase was washed with H₂O, and brine. After drying by Na₂SO₄, it was evaporated. The purification was done by distillation at 50° C. under <1 mm Hg. Yield: 19.8 g (71%). ¹H-NMR (400 MHz, CDCl₃) δ 8.39 (d, 1H), 6.84 (d, 1H), 2.59 (s, 3H), and 2.48 (s, 3H); LC/MS: m/z 141 (M+1)⁺.

### Step B: 2-[2-(Methylthio)-4-pyrimidinyl]-1-(3-nitrophenyl)ethanone

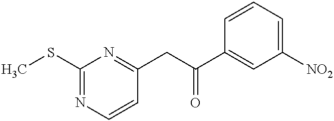

To a 500 mL three necked round bottom flask, 4-methyl-2-(methylthio)pyrimidine (9.0 g, 64 mmol) and methyl 3-nitro benzoate were placed. In argon atmosphere, dry THF (130 mL) was added. Then the mixture was cooled at −78° C. From a dropping funnel, 1 M LiHMDS in THF (64 mL) was added keeping the temperature less than −70° C. After 1.5 h at this temperature, the reaction mixture was gradually warmed up to 0° C. over an additional 1.5 h. The reaction was quenched by saturated NH₄Cl. The mixture was extracted with AcOEt (three times). The organic phase was washed by H₂O and brine. After evaporation, the crude mixture was purified with silica column chromatography (Hexane: AcOEt=5:1˜1:1) to give pure desired aldol compound, 5.9 g (30%). ¹H-NMR (400 MHz, DMSO-d6) δ 8.31-8.51 (m, 3H), 7.78-7.89 (m, 2H), 7.10-7.24 (m, 2H), 6.70 (s, 1H), and 2.60 (s, 3H).

### Step C: 2-Bromo-2-[2-(methylthio)-4-pyrimidinyl]-1-(3-nitrophenyl)ethanone

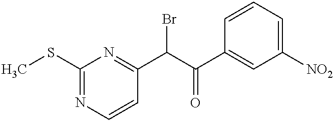

To a mixture of AcOH (100 mL) and 2-[2-(methylthio)-4-pyrimidinyl]-1-(3-nitrophenyl)ethanone (5.9 g, 20 mmol) under vigorous stirring, Br₂ (3.3 g) in AcOH (40 mL) solution was added slowly. After checking by TLC, the reaction mixture was evaporated. To the residue, DCM (150 mL) and 1N NaOH solution (100 mL) were added. Then the mixture was extracted and the organic phase was washed with 1N NaOH and H₂O and brine. After evaporation, the crude mixture was purified by silica gel chromatography to give pure desired compound (7.1 g, 96%). This intermediate was used in the next step without further purification.

### Step D: 2-(Methylthio)-4-[4-(3-nitrophenyl)-1,3-oxazol-5-yl]pyrimidine

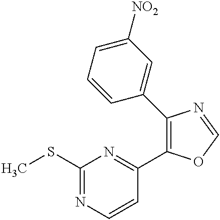

To a mixture of 2-bromo-2-[2-(methylthio)-4-pyrimidinyl]-1-(3-nitrophenyl)ethanone and formamide (125 mL), conc. sulfuric acid (12 drops) was added. Then the mixture was heated at 125° C. After 45 min, the mixture was allowed to cool to rt, then DCM (200 mL) and H₂O (300 mL) were added. The reaction mixture was extracted by DCM (additional 100 mL). The combined organic phase was washed by H₂O (300 mL) and brine (200 mL). After evaporation, the obtained crude product was purified by silica gel column chromatography (Hexane: AcOEt=3:1˜2:1) to give pure target compound (1.98 g, 32%). LC/MS: m/z 314 (M+1)⁺.

### Step E: 2-(Methylsulfinyl)-4-[4-(3-nitrophenyl)-1,3-oxazol-5-yl]pyrimidine

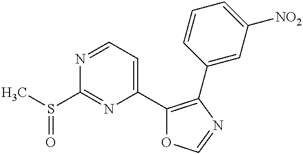

To a solution of 2-(methylthio)-4-[4-(3-nitrophenyl)-1,3-oxazol-5-yl]pyrimidine (1.98 g, 6.3 mmol), mCPBA (1.3 g, 7.5 mmol) was added under ice cooling. Then the ice bath was removed and the reaction was stirred for 2 hr. The mixture was washed with 1N NaOH and H₂O. The organic phase was evaporated. The crude mixture was purified by silica column chromatography (DCM-3% MeOH) to give a mixture of the desired product and the starting material. ¹H-NMR (400 MHz, CDCl3) δ 9.11 (1H, d, J=5 Hz), 8.92 (1H, m), 8.72 (1H, m), 8.31 (1H, m), 7.95 (1H, d, J=5 Hz), 7.75 (1H, t, J=8Hz), and 2.81 (s, 3H).

### Step F: N-(3-fluorophenyl)-4-[4-(3-nitrophenyl)-1,3-oxazol-5-yl]-2-pyrimidinamine

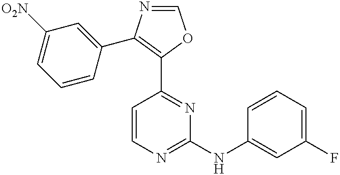

To a stirred suspension containing 400 mg (1.2 mmol) of 2-(methylsulfinyl)-4-[4-(3-nitrophenyl)-1,3-oxazol-5-yl]pyrimidine and 3 mL of DMA was added 403 mg (3.6 mmol) of 3-fluorophenyl amine and 2 drops of conc HCl. The suspension was heated at 140° C. for 16 h, and then partitioned between H₂O and DCM. The organics were washed with saturated aqueous NaHCO₃, dried over MgSO₄, and the solvents were removed under reduced pressure. The residue was subjected to silica gel chromatography to give 125 mg (28%) of the title compound of Step F: ¹H-NMR (400 MHz, CDCl₃) δ 8.62 (d, J=5.3 Hz, 1H), 8.44 (d, J=7.7 Hz, 1H), 8.25 (dd, J=8.2 and 1.4 Hz, 1H), 8.10 (s, 1H), 7.66-7.73 (m, 2H), 7.62 (t, J=8.2 Hz, 1H), 7.19-7.25 (m, 4H), and 6.74 (s, 1H).

### Step G: 4-[4-(3-Aminophenyl)-1,3-oxazol-5-yl]-N-(3-fluorophenyl)-2-pyrimidinamine

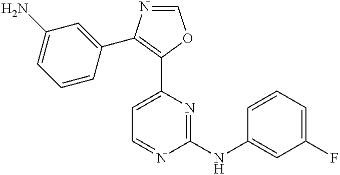

A slurry containing 125 mg (0.33 mmol) of N-(3-fluorophenyl)-4-[4-(3-nitrophenyl-1,3-oxazol-5-yl]-2-pyrimidinamine, 20 mg of 5% by weight Pt on carbon, and 5 mL of EtOH was treated under an atmosphere of H₂ gas for 12 h. The reaction mixture was filtered through a pad of Celite, eluting with EtOH and EtOAc, and the solvent was removed under reduced pressure to give 115 mg (100%) of the title compound of Step G: ¹H-NMR (400 MHz, CDCl₃) δ 8.30-8.42 (m, 1H), 8.04 (s, 1H), 7.57 (d, J=6.4 Hz, 1H), 7.41-7.47 (m, 2H), 7.37 (s, 1H), 7.15-7.26 (m, 1H), 6.99-7.10 (m, 3H), 6.79-6.91 (m, 1H), 6.66-8.74 (m, 1H), and 3.83 (s, 2H).

### Step H: 2,6-Difluoro-N-[3-(5-{2-[(3-fluorophenyl)amino]-4-pyrimidinyl}-1,3-oxazol-4-yl)phenyl]benzamide

To a solution containing 57 mg (0.16 mmol) of 4-[4-(3-aminophenyl)-1,3-oxazol-5-yl]-N-(3-fluorophenyl)-2-pyrimidinamine and 2 mL of THF was added 35 μL (0.28 mmol) of 2,6-difluorobenzoyl chloride. The reaction was allowed to stir for 4 h, then 20 mg of PS-trisamine was added and the mixture was allowed to stir overnight and 0.5 mL of TEA was added. The reaction mixture was filtered and the solvents were removed under reduced pressure. The residue was subjected to silica gel chromatography and further purified by trituration to give 2 mg of 2,6-difluoro-N-[3-(5-{2-[(3-fluorophenyl)amino]-4-pyrimidinyl}-1,3-oxazol-4-yl)phenyl]benzamide: ¹H-NMR (400 MHz, CD₃OD) δ 8.79 (s, 1H), 8.55 (d, J=5.3 Hz, 1H), 8.42 (s, 1H), 7.73 (d, J=8.1 Hz, 1H), 7.47-7.58 (m, 3H), 7.42 (t, J=7.9 Hz, 1H), 7.28 (d, J=7.5 Hz, 1H), 7.21 (d, J=5.1 Hz, 2H), 7.03-7.13 (m, 4H), and 6.60 (td, J=8.7 and 2.1 Hz, 1H); LC-MS (M+H⁺): 488.08.

### Example 142

### N-[3-(5-{2-[3-Chloro-4-{[2-(dimethylamino)ethyl]oxy}phenyl)amino]-4-pyrimidinyl}-2-methyl-1,3-oxazol-4-yl)phenyl]-2,5-difluorobenzamide

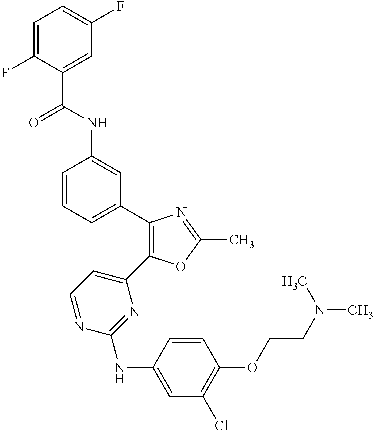

### Step A: N-{3-[5-(2-Chloro-4-pyrimidinyl)-2-methyl-1,3-oxazol-4-yl]phenyl}-2,5-difluorobenzamide

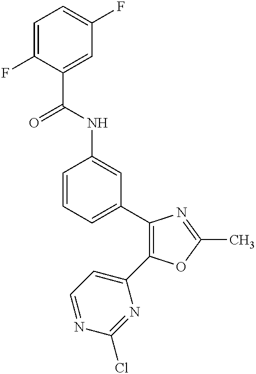

A solution of N-{3-[bromo(2-chloro-4-pyrimidinyl)acetyl]phenyl}-2,5-difluorobenzamide (150 mg, 0.387 mmol), prepared by a procedure analogous to Example 50, Step B, and acetamide (80 mg, 1.35 mmol) in THF (1.5 mL) was heated in a personal chemistry microwave at 140° C. for 30 min. The reaction mixture was diluted with DCM and MeOH and concentrated onto silica gel. Purification by column chromatography (10-100% 1:9:90 ammonium hydroxide:MeOH:DCM in DCM) provided 0.081 g (49%) as an off-white solid. MS (ESI): 427.03 [M+H]⁺.

### Step B: N-[3-(5-{2-[(3-Chloro-4-{[2-(dimethylamino)ethyl]oxy}phenyl)amino]-4-pyrimidinyl}-2-methyl-1,3-oxazol-4-yl)phenyl]-2,5-difluorobenzamide

A solution of N-{3-[5-(2-chloro-4-pyrimidinyl)-2-methyl-1,3-oxazol-4-yl]phenyl}-2,5-difluorobenzamide (60 mg, 0.141 mmol) and {2-[(4-amino-2-chlorophenyl)oxy]ethyl}dimethylamine hydrochloride (35 mg, 0.141 mmol), in i-PrOH (1.5 mL) was treated with concentrated HCl (3 drops) and heated in a personal chemistry microwave at 160° C. for 10 min. The reaction mixture was diluted with DCM and concentrated onto silica gel. Purification by column chromatography (10-100% 1:9:90 NH₄OH:MeOH:DCM in DCM) provided 0.025 g (29%) as a yellow solid. ¹H-NMR (400 MHz, CDCl₃) δ 9.19 (brs, 1H), 8.64 (brs, 1H), 8.58 (d, 1H, J=15.4 Hz), 8.46 (d, 1H, J=5.3 Hz), 7.86 (m, 1H), 7.84 (d, 1H, J=8.0 Hz), 7.75 (s, 1H), 7.55 (d, 1H, J=8.8 Hz), 7.43 (t, 1H, J=7.9 Hz), 7.29 (m, 1H), 7.24-7.16 (m, 2H), 7.09 (d, 1H, J=5.3 Hz), 6.83 (d, 1H, J=9.2 Hz), 4.33 (brs, 2H), 3.20 (brs, 2H), 2.73 (brs, 6H), and 2.63 (s, 3H); MS (ESI): 605.3 [M+H]⁺.

### Example 143

### N-[3-(2-Amino-5-{2-[(3-chloro-4-{[2-(dimethylamino)ethyl]oxy}phenyl)amino]-4-pyrimidinyl}-1,3-oxazol-4-yl)phenyl]-2,6-difluorobenzamide trifluoroacetate

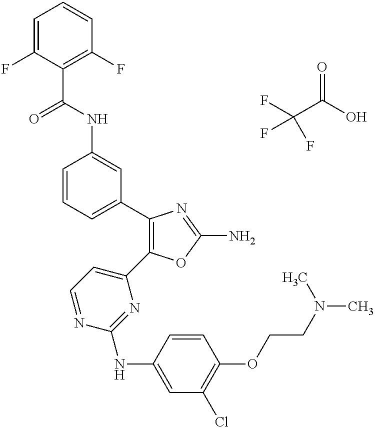

### Step A: N-{3-[2-Amino-5-(2-chloro-4-pyrimidinyl)-1,3-oxazol-4-yl]phenyl}-2,6-difluorobenzamide

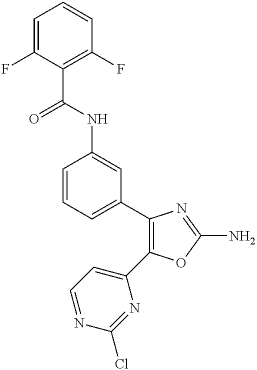

A solution of N-{3-[bromo(2-chloro-4-pyrimidinyl)acetyl]phenyl}-2,6-difluorobenzamide (500 mg, 1.29 mmol), prepared by a procedure analogous to Example 1, Step C, and urea (500 mg, 8.3 mmol) in 1,4-dioxane (5.0 mL) was heated in a personal chemistry microwave at 150° C. for 40 min. The reaction mixture was diluted with DCM and concentrated onto silica gel. Purification by column chromatography (50-100% EtOAc in hexanes) provided 0.155 g (28%) as an orange-tan solid. MS (ESI): 428.1 [M+H]⁺.

### Step B: N-[3-(2-Amino-5-{2-[(3-chloro-4-{[2-(dimethylamino)ethyl]oxy}phenyl)amino]-4-pyrimidinyl}-1,3-oxazol-4-yl)phenyl]-2,6-difluorobenzamide trifluoroacetate

A solution of N-{3-[5-(2-chloro-4-pyrimidinyl)-2-methyl-1,3-oxazol-4-yl]phenyl}-2,5-difluorobenzamide (55 mg, 0.129 mmol) and {2-[(4-amino-2-chlorophenyl)oxy]ethyl}dimethylamine hydrochloride (29 mg, 0.129 mmol), in i-PrOH (2 mL) and trifluoroethanol (2 mL) was treated with concentrated HCl (2 drops) and heated at 90° C. for 16 h. The reaction mixture was diluted with DCM and concentrated onto silica gel. Purification by column chromatography (10-100% 1:9:90 NH₄OH:MeOH:DCM in DCM), then preparative HPLC (5-50% acetonitrile in 0.1% TFA/H₂O over 20 min) provided 0.040 g (43%) as a yellow solid. ¹H-NMR (400 MHz, CDCl₃) δ 10.9 (s, 1H), 9.65 (brs, 1H), 9.45 (s, 1H), 8.42 (brs, 1H), 8.38 (d, 1H, J=5.3 Hz), 7.67 (m, 2H), 7.61-7.50 (m, 2H), 7.39 (s, 1H), 7.33 (t, 1H, J=7.8 Hz), 7.22 (t, 2H, J=8.0 Hz), 6.93 (d, 1H, J=8.8 Hz), 6.79 (d, 1H, J=5.3 Hz), 4.29 (t, 2H, J=4.8 Hz), 3.51 (m, 2H), and 2.88 (d, 6H); MS (ESI): 606.2 [M+H]⁺.

### Biological Examples

Compounds of the present invention were tested for ErbB family and B-RAF protein tyrosine kinase inhibitory activity in substrate phosphorylation assays and cell proliferation assays.

### A. Enzyme Assays—EGFR and ErbB-2 Assays:

Compounds of the present invention were tested for EGFR and ErbB-2 protein tyrosine kinase inhibitory activity in substrate phosphorylation assays using enzymes purified from a baculovirus expression system. Reagent production was conducted essentially as described in Brignola, P. S., et al., (2002) J. Biol. Chem. 277(2):1576-1585.

The method measures the ability of the isolated enzyme to catalyse the transfer of the gamma-phosphate from ATP onto the tyrosine residue of a biotinylated synthetic peptide referenced “Peptide C” in Brignola, P. S., et al., (2002) J. Biol. Chem. 277(2):1576-1585. The extent of tyrosine phosphorylation was measured using an anti-phosphotyrosine antibody, and quantified by homogenous time-resolved fluorescence (HTRF).

Reactions were performed in black 384-well polystyrene flat-bottom plates in a final volume of 20 μl. Assays were performed by adding 10 μl of each of the following solutions, Substrate Mix and Enzyme Mix: The Substrate Mix contained 100 mM 3-[N-morpholino]propanesulfonic acid (MOPS) (pH 7.5), 2 mM MnCl₂, 20 μM ATP, 0.01% Tween-20, 0.1 mg/mL (BSA), 0.8 uM peptide substrate, and 1 mM dithiothreitol. The Enzyme Mix contained 100 mM MOPS (pH 7.5); 0.01% Tween-20; 0.1 mg/mL BSA, and either 0.8 nM EGFR, 10 nM ErbB2, or 1 nM ErbB4

During the course of these studies, two separate methods have been used to measure the potency of compounds. In Method A (indicated as “(A)” in the tables below), the Enzyme Mix was added to the compound plates and the plates were incubated at 20° C. for 1 hr. The reactions were then started by adding the Substrate Mix. In Method B (indicated as “(B)” in the tables below), the Substrate Mix was added to the compound plates first, then the reaction was started by adding the Enzyme Mix.

After initiating the reaction with either method describe above, the reactions were allowed to proceed for 90 minutes at 20° C. The reactions were then terminated by the addition of 20 μL 100 mM EDTA to each well. 40 μL/well of HTRF detection mix were added to the assay plates. The final concentrations of the detection reagents were: 100 mM HEPES (pH7.5), 0.1 mg/mL BSA, 15 nM streptavidin-labeled allophycocyanin (PerkinElmer), and 1 nM europium-labeled anti-phosphotyrosine antibody (PerkinElmer). Assay plates were left unsealed and were counted in a Wallac Multilabel Counter 1420 (PerkinElmer).

Compounds under analysis were dissolved in dimethylsulfoxide (DMSO) to 1.0 mM and serially diluted 1 to 3 with DMSO through twelve dilutions. 1 μL of each concentration was transferred to the corresponding well of an assay plate. This creates a final compound concentration range from 0.00027 to 47.6 μM.

The data for dose responses were plotted as % Inhibition calculated with the data reduction formula 100*(1−(U1−C2)/(C₁−C₂)) versus concentration of compound where U1 is the unknown value, C1 is the average control value obtained for 4.76% DMSO, and C2 is the average control value obtained for 0.035 M EDTA. Data were fitted with a curve described by:

y=((Vmax*x)/(K+x))+Y2

where Vmax is the upper asymptote, Y2 is the Y intercept, and K is the IC50. The results for each compound were recorded as pIC50s, calculated as follows:

pIC50=−Log 10(K)

Many of the exemplified compounds Examples 1-143 were run in the recited assay and the results are reported in the following Table 1. In the following table:
“+” indicates no pIC50 measurement greater than 6 against EGFR,
“++” indicates at least one pIC50 measurement greater than 6 against EGFR but no measurement greater than pIC50 of 7; and
“+++” indicates at least one pIC50 measurement of greater than 7 against EGFR.

TABLE 1
EGFR Activity

| | Activity |
|---|---|
| Example | (Method) |
| 1 | +++ (A) |
| 2 | +++ (A) |
| 3 | +++ (A) |
| 4 | +++ (A) |
| 5 | +++ (A) |
| 6 | +++ (A) |
| 7 | +++ (A) |
| 8 | +++ (A) |
| 9 | +++ (A) |
| 10 | +++ (A) |
| 11 | +++ (A) |
| 12 | +++ (A) |
| 13 | +++ (A) |
| 14 | +++ (A) |
| 15 | +++ (A) |
| 16 | +++ (A) |
| 17 | +++ (A) |
| 18 | +++ (A) |
| 19 | +++ (A) |
| 20 | +++ (A) |
| 21 | +++ (A) |
| 22 | +++ (A) |
| 23 | +++ (A) |
| 24 | +++ (A) |
| 25 | +++ (A) |
| 26 | +++ (A) |
| 27 | +++ (A) |
| 28 | +++ (A) |
| 29 | +++ (A) |
| 30 | +++ (A) |
| 31 | +++ (A) |
| 32 | +++ (A) |
| 33 | +++ (A) |
| 34 | +++ (A) |
| 35 | +++ (A) |
| 36 | +++ (A) |
| 37 | +++ (A) |
| 38 | +++ (A) |
| 39 | +++ (A) |
| 40 | +++ (A) |
| 41 | +++ (A) |
| 42 | +++ (A) |
| 43 | +++ (A) |
| 44 | +++ (A) |
| 45 | +++ (A) |
| 46 | +++ (A) |
| 47 | +++ (A) |
| 48 | ++(A) |
| 49 | +++ (A) |
| 50 | +++ (A) |
| 51 | +++ (A) |
| 52 | +++ (A) |
| 53 | +++ (A) |
| 54 | +++ (A) |
| 55 | +++ (A) |
| 56 | +++ (A) |
| 57 | +++ (A) |
| 58 | +++ (A) |
| 59 | +++ (A) |
| 60 | +++ (A) |
| 61 | +++ (A) |
| 62 | +++ (A) |
| 63 | +++ (A) |
| 64 | +++ (A) |
| 65 | +++ (A) |
| 66 | +++ (A) |
| 67 | +++ (A) |
| 68 | +++ (A) |
| 69 | +++ (A) |
| 70 | +++ (A) |
| 71 | +++ (A) |
| 72 | +++ (A) |
| 73 | +++ (A) |
| 74 | +++ (A) |
| 75 | +++ (A) |
| 76 | +++ (A) |
| 77 | +++ (A) |
| 78 | +++ (A) |
| 79 | +++ (A) |
| 80 | +++ (A) |
| 81 | +++ (A) |
| 82 | +++ (A) |
| 83 | +++ (A) |
| 84 | +++ (A) |
| 85 | +++ (A) |
| 86 | +++ (A) |
| 87 | ++ (B) |
| 88 | ++ (A) |
| 89 | +++ (A) |
| 90 | +++ (A) |
| 91 | +++ (A) |
| 92 | +++ (A) |
| 93 | +++ (A) |
| 94 | +++ (A) |
| 95 | +++ (A) |
| 96 | +++ (A) |
| 97 | +++ (A) |
| 98 | +++ (A) |
| 99 | +++ (A) |
| 100 | +++ (A) |
| 101 | +++ (A) |
| 102 | +++ (A) |
| 103 | +++ (A) |
| 104 | +++ (A) |
| 105 | +++ (A) |
| 106 | +++ (A) |
| 107 | +++ (A) |
| 108 | +++ (A) |
| 109 | +++ (A) |
| 110 | +++ (A) |
| 111 | +++ (A) |
| 112 | +++ (A) |
| 113 | +++ (A) |
| 114 | +++ (A) |
| 115 | +++ (A) |
| 116 | +++ (A) |
| 117 | +++ (A) |
| 119 | +++ (A) |
| 120 | +++ (A) |
| 121 | +++ (A) |
| 122 | +++ (A) |
| 123 | +++ (A) |
| 124 | +++ (A) |
| 125 | +++ (A) |
| 126 | +++ (A) |
| 127 | +++ (A) |
| 128 | +++ (A) |
| 129 | ++(A) |
| 130 | +++ (A) |
| 131 | +++ (A) |
| 132 | +++ (A) |
| 133 | ++(A) |
| 134 | +++ (A) |
| 135 | +++ (A) |
| 136 | +++ (A) |
| 137 | +++ (A) |
| 138 | ++(A) |
| 139 | +++ (A) |
| 140 | +++ (A) |
| 141 | + (B) |
| 142 | +++ (A) |

Many of the exemplified compounds Examples 1-143 were run in the recited assay and the results are reported in the following Table 2. In the following table:
“+” indicates no pIC50 measurement greater than 6 against ErbB2
“++” indicates at least one pIC50 measurement greater than 6 against ErbB2 but no measurement greater than pIC50 of 7; and
“+++” indicates at least one pIC50 measurement of greater than 7 against ErbB2.

TABLE 2
ErbB2 Activity

| | Activity |
|---|---|
| Example | (Method) |
| 1 | +++ (A) |
| 2 | +++ (A) |
| 3 | +++ (A) |
| 4 | +++ (A) |
| 5 | +++ (A) |
| 6 | +++ (A) |
| 7 | +++ (A) |
| 8 | +++ (A) |
| 9 | +++ (A) |
| 10 | +++ (A) |
| 11 | +++ (A) |
| 12 | +++ (A) |
| 13 | +++ (A) |
| 14 | +++ (A) |
| 15 | +++ (A) |
| 16 | +++ (A) |
| 17 | +++ (A) |
| 18 | +++ (A) |
| 19 | +++ (A) |
| 20 | +++ (A) |
| 21 | +++ (A) |
| 22 | +++ (A) |
| 23 | +++ (A) |
| 24 | +++ (A) |
| 25 | +++ (A) |
| 26 | +++ (A) |
| 27 | +++ (A) |
| 28 | +++ (A) |
| 29 | +++ (A) |
| 30 | +++ (A) |
| 31 | +++ (A) |
| 32 | +++ (A) |
| 33 | +++ (A) |
| 34 | +++ (A) |
| 35 | +++ (A) |
| 36 | +++ (A) |
| 37 | +++ (A) |
| 38 | +++ (A) |
| 39 | +++ (A) |
| 40 | +++ (A) |
| 41 | +++ (A) |
| 42 | +++ (A) |
| 43 | +++ (A) |
| 44 | +++ (A) |
| 45 | +++ (A) |
| 46 | +++ (A) |
| 47 | +++ (A) |
| 48 | +(A) |
| 49 | ++(A) |
| 50 | +++ (A) |
| 51 | +++ (A) |
| 52 | +++ (A) |
| 53 | +++ (A) |
| 54 | +++ (A) |
| 55 | +++ (A) |
| 56 | +++ (A) |
| 57 | +++ (A) |
| 58 | +++ (A) |
| 59 | +++ (A) |
| 60 | +++ (A) |
| 61 | +++ (A) |
| 62 | +++ (A) |
| 63 | +++ (A) |
| 64 | ++(A) |
| 65 | +++ (A) |
| 66 | +++ (A) |
| 67 | +++ (A) |
| 68 | +++ (A) |
| 69 | +++ (A) |
| 70 | +++ (A) |
| 71 | +++ (A) |
| 72 | +++ (A) |
| 73 | +++ (A) |
| 74 | +++ (A) |
| 75 | +++ (A) |
| 76 | +++ (A) |
| 77 | +++ (A) |
| 78 | +++ (A) |
| 79 | +++ (A) |
| 80 | +++ (A) |
| 81 | +++ (A) |
| 82 | +++ (A) |
| 83 | +++ (A) |
| 84 | +++ (A) |
| 85 | +++ (A) |
| 86 | ++(A) |
| 87 | ++ (B) |
| 88 | ++ (B) |
| 89 | +++ (A) |
| 90 | +++ (A) |
| 91 | +++ (A) |
| 92 | +++ (A) |
| 93 | +++ (A) |
| 94 | +++ (A) |
| 95 | +++ (A) |
| 96 | +++ (A) |
| 97 | +++ (A) |
| 98 | +++ (A) |
| 99 | +++ (A) |
| 100 | +++ (A) |
| 101 | +++ (A) |
| 102 | +++ (A) |
| 103 | +++ (A) |
| 104 | +++ (A) |
| 105 | +++ (A) |
| 106 | +++ (A) |
| 107 | +++ (A) |
| 108 | +++ (A) |
| 109 | +++ (A) |
| 110 | +++ (A) |
| 111 | +++ (A) |
| 112 | +++ (A) |
| 113 | +++ (A) |
| 114 | +++ (A) |
| 115 | +++ (A) |
| 116 | +++ (A) |
| 117 | +++ (A) |
| 119 | +++ (A) |
| 120 | +++ (A) |
| 121 | +++ (A) |
| 122 | +++ (A) |
| 123 | +++ (A) |
| 124 | +++ (A) |
| 125 | +++ (A) |
| 126 | +++ (A) |
| 127 | +++ (A) |
| 128 | +++ (A) |
| 129 | +++ (A) |
| 130 | +++ (A) |
| 131 | +++ (A) |
| 132 | +++ (A) |
| 133 | ++(A) |
| 134 | +++ (A) |
| 135 | +++ (A) |
| 136 | +++ (A) |
| 137 | +++ (A) |
| 138 | +(A) |
| 139 | +++ (A) |
| 140 | +++ (A) |
| 141 | + (B) |
| 142 | +++ (A) |

### B. Enzyme Assays—B-Raf Assays:

Compounds of the present invention were tested for B-Raf protein serine kinase inhibitory activity in a B-Raf Accelerated MEK ATPase assay (BRAMA). The BRAMA assay is a high sensitivity assay which measures an intrinsic MEK-mediated ATP hydrolysis uncoupled from downstream ERK phosphorylation by coupling the formation of ADP to NADH oxidation through the enzymes pyruvate kinase and lactate dehydrogenase. When ADP production is initiated by addition of catalytic amounts of an activated Raf enzyme and non-phosphorylated MEK, one observes robust ADP production concomitant with Raf-mediated phosphorylation of MEK.

Method A: Specific details of the assay are disclosed in: C. Rominger, M. Schaber, E. May. Assay for B-Raf Activity Based on Intrinsic MEK ATPase Activity. Statutory Invention Registration Ser. No. 11/084,993 (March, 2005).
Method B: The method is the same as method A, but includes the following changes:
1) the assay was performed with a final MEK concentration of 150 nM and 2) the assay was read as single end point instead of a kinetic read.

Many of the exemplified compounds Examples 1-143 were run in the recited assay and the results are reported in the following Table 3. In the following table:
“+” indicates no pIC50 measurement greater than 6 against B-Raf
“++” indicates at least one pIC50 measurement greater than 6 against B-Raf but no measurement greater than pIC50 of 7; and
“+++(A) (A)” indicates at least one pIC50 measurement of greater than 7 against B-Raf.

TABLE 3
B-Raf Activity

| | Activity |
|---|---|
| Example | (Method) |
| 2 | +++ (A) |
| 3 | +++ (A) |
| 4 | +++ (B) |
| 5 | +++ (B) |
| 6 | +++ (A) |
| 7 | +++ (A) |
| 8 | +++ (A) |
| 9 | +++ (A) |
| 10 | +++ (A) |
| 11 | +++ (A) |
| 12 | +++ (A) |
| 13 | +++ (A) |
| 14 | +++ (A) |
| 15 | +++ (A) |
| 16 | +++ (A) |
| 17 | +++ (A) |
| 18 | +++ (A) |
| 19 | +++ (A) |
| 20 | +++ (A) |
| 22 | +++ (A) |
| 23 | +++ (B) |
| 24 | +++ (B) |
| 25 | +++ (A) |
| 26 | +++ (A) |
| 27 | +++ (A) |
| 28 | +++ (A) |
| 29 | +++ (A) |
| 30 | +++ (A) |
| 31 | +++ (A) |
| 32 | +++ (A) |
| 33 | +++ (B) |
| 34 | +++ (A) |
| 35 | +++ (A) |
| 36 | +++ (A) |
| 37 | +++ (A) |
| 38 | +++ (A) |
| 39 | +++ (A) |
| 40 | +++ (A) |
| 41 | +++ (A) |
| 42 | ++ (A) |
| 43 | +++ (A) |
| 44 | +++ (A) |
| 45 | +++ (A) |
| 46 | +++ (A) |
| 47 | +++ (A) |
| 48 | + (A) |
| 49 | + (A) |
| 50 | +++ (A) |
| 51 | +++ (A) |
| 52 | +++ (A) |
| 53 | +++ (A) |
| 54 | +++ (A) |
| 56 | +++ (A) |
| 57 | +++ (A) |
| 58 | +++ (A) |
| 61 | +++ (A) |
| 63 | +++ (A) |
| 64 | +++ (A) |
| 65 | +++ (A) |
| 66 | + (A) |
| 68 | +++ (A) |
| 69 | + (A) |
| 70 | +++ (A) |
| 71 | +++ (A) |
| 72 | +++ (A) |
| 73 | +++ (A) |
| 74 | +++ (A) |
| 75 | +++ (B) |
| 76 | +++ (A) |
| 77 | +++ (A) |
| 78 | +++ (A) |
| 79 | +++ (A) |
| 80 | +++ (B) |
| 81 | +++ (A) |
| 82 | +++ (A) |
| 83 | +++ (A) |
| 84 | +++ (A) |
| 85 | +++ (A) |
| 86 | +++ (A) |
| 91 | +++ (A) |
| 92 | +++ (A) |
| 93 | +++ (A) |
| 94 | +++ (A)B |
| 95 | +++ (A) |
| 96 | +++ (A) |
| 97 | +++ (A) |
| 98 | +++ (A) |
| 99 | +++ (A) |
| 100 | +++ (A) |
| 101 | +++ (A) |
| 102 | +++ (A) |
| 103 | +++ (A) |
| 104 | +++ (A) |
| 105 | +++ (A) |
| 106 | ++ (A) |
| 107 | +++ (A) |
| 108 | +++ (A) |
| 109 | +++ (A) |
| 110 | +++ (A) |
| 111 | +++ (A) |
| 112 | +++ (A) |
| 113 | +++ (A) |
| 114 | +++ (A) |
| 115 | ++ (A) |
| 116 | +++ (A) |
| 117 | +++ (A) |
| 118 | +++ (A) |
| 119 | +++ (A) |
| 120 | +++ (A) |
| 121 | +++ (A) |
| 122 | +++ (A) |
| 123 | +++ (A) |
| 124 | +++ (A) |
| 125 | +++ (A) |
| 126 | +++ (A) |
| 127 | +++ (A) |
| 128 | +++ (A) |
| 129 | +++ (A) |
| 130 | +++ (A) |
| 131 | +++ (A) |
| 132 | +++ (A) |
| 133 | +++ (A) |
| 134 | +++ (A) |
| 135 | +++ (A) |
| 136 | +++ (A) |
| 137 | +++ (A) |
| 138 | +++ (A) |
| 139 | +++ (A) |
| 140 | +++ (A) |
| 142 | +++ (A) |

C. Cellular assays

### Method A: Methylene Blue Growth Inhibition Assay

Human breast (BT474) and head and neck (HN5) tumor cells were cultured in low glucose DMEM (Life Technologies 12320-032) containing 10% fetal bovine serum (FBS). Human colon tumor cells (Colo205) were cultured in DMEM (Invitrogen, 10564) containing 10% FBS. The SV40 transformed human mammary epithelial cell line HB4a was transfected with either human H-ras cDNA (HB4a r4.2) or the human c-ErbB2 cDNA (HB4a c5.2). The HB4a clones were cultured in RPMI containing 10% FBS, insulin (5 g/mL), hydrocortisone (5 g/mL), supplemented with the selection agent hygromycin B (50 g/mL). All lines were grown in a humidified incubator at 37° C. in 95% air, 5% CO₂. Cells were harvested using trypsin/EDTA, counted using a haemocytometer, and plated in 100 μL of the appropriate media, at the following densities, in a 96-well tissue culture plate (Falcon 3075): BT474 10,000 cells/well, HN5 3,000 cells/well. The next day, compounds were diluted in DMEM containing 100 mg/mL gentamicin, at twice the final required concentration, from 10 mM stock solutions in DMSO. 100 μl/well of these dilutions were added to the 100 μL of media currently on the cell plates. Medium containing 0.6% DMSO was added to control wells. Compounds diluted in DMEM were added to all cell lines. The final concentration of DMSO in all wells was 0.3%. Cells were returned to the incubator (37° C., 10% CO₂) for 3 days. Medium was then removed by aspiration. Cell biomass was estimated by staining cells with 90 μL/well methylene blue (Sigma M9140, 0.5% in 1:1 EtOH:H₂O), and incubation at room temperature for at least 30 minutes. Stain was removed, and the plates rinsed by immersion in deionized H₂O and air-dried. To release stain from the cells 100 μL of solubilization solution were added (1% N-lauroyl sarcosine, sodium salt, Sigma L5125, in PBS), and plates were shaken gently for about 30 minutes. Optical density at 620 nM was measured on a microplate reader. Percent inhibition of cell growth was calculated relative to vehicle-treated control wells. Concentration of compound that inhibits 50% of control cell growth (IC₅₀) was interpolated using nonlinear regression (Levenberg-Marquardt) and the equation,

y=Vmax*(1−(x/(K+x)))+Y2, where “K” was equal to the IC₅₀.

### Method B: CellTiter-Glo® Growth Inhibition Assays

Human breast tumor cells (BT474) were cultured in RPMI, (Invitrogen, 22400) containing 10% fetal bovine serum (FBS). Human head and neck tumor cells (HN5) were cultured in low glucose DMEM (Invitrogen, 12320) containing 10% FBS. Human colon tumor cells (Colo205) were cultured in DMEM (Invitrogen, 10564) containing 10% FBS. Human melanoma cancer cells (SK-MEL-28) were incubated in DMEM (Invitrogen). All cell lines were maintained at 37° C. in a humidified 5% CO₂, 95% air incubator. Cells were harvested using trypsin/EDTA, counted using a haemocytometer, and plated in 30 μL of the appropriate media described above, at the following densities, in a half-area, black-walled, 96-well tissue culture plate (Corning 3882): BT474 3,000 cells/well, HN5 500 cells/well, Colo205 3,000 cells/well, and SK-MEL-28 500 cells/well). The next day, compounds were diluted in DMEM containing 100 mg/mL gentamicin, from 10 mM stock solutions in DMSO. 30 μL/well of these dilutions were added to the 30 μL/well of media currently on the cell plates. Medium containing 0.6% DMSO was added to control wells. The final concentration of DMSO in all wells was 0.3%. Cells were incubated at 37° C., 5% CO₂ for 3 days. Cell biomass was estimated using CellTiter-Glo® reagent (Promega G7571). Briefly, plates were removed from the incubator and allowed to equilibrate to rt for 30 minutes. 25-60 μL of CellTiter-Glo® reagent were added to each well of the treated cells and plates were shaken on an orbital plate shaker for 2 mins. Plates were incubated without shaking for 30 more mins and read in a luminometer with an integration time of 0.5 seconds per well. Percent inhibition of cell growth was calculated relative to vehicle-treated control wells. Concentration of compound that inhibits 50% of vehicle control cell growth (IC50) was interpolated using nonlinear regression (Levenberg-Marquardt) and the equation, y=V max*(1−(x/(K+x)))+Y2, where “K” was equal to the IC50.

### Method C: Cell Growth Inhibition Assays

Human breast tumor cells (BT474) were cultured in RPMI (Mediatech 50-011-PB), containing 10% FBS (JRH Biosciences 12176) and 1% penicillin-streptomycin (Invitrogen 15140). Human head and neck tumor cells (HN5) were cultured in high glucose DMEM (Mediatech 50-013-PB) containing 10% FBS and 1% penicillin-streptomycin. Human colon tumor cells (Colo205) were cultured in RPMI (Mediatech 50-020-PB) containing 10% FBS and 1% penicillin-streptomycin. Human melanoma cancer cells (SK-MEL-28) were cultured in EMEM with nonessential amino acids (Mediatech 50-011-PB) containing 10% FBS, 1% sodium pyruvate (JT Baker 3354-04), and 1% penicillin-streptomycin. Human melanoma cancer cells (A375P F11s) were cultured in RPMI (Mediatech 50-020-PB) containing 10% FBS, 1% sodium pyruvate, and 1% penicillin-streptomycin. Human colon cancer cells (HT-29) were cultured in high glucose DMEM (Mediatech 50-013-PB) containing 10% FBS and 1% penicillin-streptomycin. Human melanoma cancer cells (SK-MEL-3) were cultured in McCoy's 5A (50-025-PB) containing 15% FBS, and 1% penicillin-streptomycin. All cell lines were maintained at 37° C. in a humidified 5% CO₂, 95% air incubator. Cells were harvested using trypsin/EDTA (Invitrogen 25200), counted using an haemocytometer, and plated. For 96-well assays (using white full-area NUNC plates cat. #136102), cells were plated in 105 μL at the following densities (cells/well): HN5, 500; Colo205, 500; SK-MEL-28, 500; A375P-F11s, 500; HT-29, 500; SK-MEL-3, 500; and BT474, 3000. For 384-well assays (white full-area NUNC plates, cat. #781080), cells were plated in 48 μL at the following densities (cells/well): HN5, 500; Colo205, 500; SK-MEL-28, 500; A375P-F11s, 500; HT29, 500; SK-MEL-3, 500; and BT474, 2000.

The next day, compounds were diluted as follow: For 96-well assays, 13.5 μL of compound in DMSO were diluted using nine (9) serial 1:3 dilutions of 4.5 μL in 9 μL of DMSO. Medium (270 μl/well of RPMI with 10% FBS and 1% penicillin-streptomycin) was added to the plates. Aliquots (7 μl) were added to cells in the final assay giving a final DMSO concentration of 0.2%. For 384-well assays, 15 μL of compound in DMSO were diluted using nine (9) serial 1:3 dilutions of 5 μL in 10 μL of DMSO, followed by a further dilution of 5 μL of compound with 95 μL of medium, of which 2 μL were added to cells in the final assay giving a final DMSO concentration of 0.2%. Cells were incubated at 37° C., 5% CO₂ for 3 days.

Total ATP was measured (as a surrogate estimate of cell number) using CellTiter-Glo® reagent (Promega G7571). Briefly, plates were removed from the incubator and allowed to equilibrate to room temperature for 30 minutes. CellTiter-Glo® (25 μL or 55 μL for 384-well or 96-well assays, respectively) reagent was added to each well and plates were shaken on an orbital plate shaker for 2 mins. Plates were incubated without shaking for a further 30 mins and read on an LJL Analyst GT reader in luminometer mode with an integration time of 0.5 seconds per well. Percent inhibition of cell growth was calculated relative to DMSO vehicle-treated control wells. Concentration of compound required to give 50% inhibition of vehicle-treated control cell growth (IC₅₀) was interpolated using a 4-parameter fit for determining IC₅₀ using the following equation: Y=A+((B−A)/(1+((C/X)̂D))) where X=IC₅₀.

Many of the compounds of Examples 1-143 were run in the recited assays and the results are reported in the following Table 4. In the following table:
“+” indicates that the compound showed activity of >1 μM in HN5 tumor cells;
“++” indicates that the compound showed activity of between 100 nM and 1 μM in HN5 tumor cells; and
“+++” indicates that the compound showed activity of less than 100 nM in HN5 tumor cells.

TABLE 4
Activity in HN5 Tumor Cells

| | Activity |
|---|---|
| Example | (Method) |
| 1 | +++ (A) |
| 2 | +++ (C) |
| 3 | +++ (A) |
| 4 | ++ (C) |
| 5 | +++ (C) |
| 6 | +++ (A) |
| 7 | +++ (B) |
| 8 | +++ (B) |
| 9 | +++ (B) |
| 10 | +++ (C) |
| 11 | +++ (C) |
| 12 | +++ (C) |
| 13 | +++ (C) |
| 14 | +++ (A) |
| 15 | +++ (B) |
| 16 | +++ (C) |
| 17 | +++ (C) |
| 18 | +++ (A) |
| 19 | +++ (B) |
| 20 | +++ (B) |
| 21 | +++ (A) |
| 22 | +++ (C) |
| 23 | +++ (C) |
| 24 | +++ (C) |
| 25 | ++ (C) |
| 26 | ++ (C) |
| 27 | +++ (C) |
| 28 | ++ (C) |
| 29 | +++ (C) |
| 30 | ++ (B) |
| 31 | +++ (C) |
| 32 | +++ (C) |
| 33 | ++ (C) |
| 34 | +++ (C) |
| 36 | +++ (C) |
| 37 | ++ (C) |
| 38 | +++ (C) |
| 39 | ++ (C) |
| 40 | ++ (C) |
| 44 | ++ (C) |
| 45 | ++ (C) |
| 48 | + (C) |
| 49 | ++ (B) |
| 50 | +++ (A) |
| 51 | + (A) |
| 52 | ++ (A) |
| 53 | +++ (A) |
| 54 | +++ (A) |
| 55 | +++ (A) |
| 56 | +++ (C) |
| 57 | +++ (A) |
| 58 | +++ (A) |
| 59 | +++ (A) |
| 60 | +++ (A) |
| 61 | +++ (A) |
| 62 | +++ (A) |
| 63 | ++ (C) |
| 64 | ++ (B) |
| 65 | +++ (A) |
| 66 | +++ (A) |
| 67 | +++ (C) |
| 68 | +++ (C) |
| 69 | ++ (A) |
| 70 | ++ (A) |
| 71 | ++ (A) |
| 72 | ++ (C) |
| 75 | ++ (C) |
| 76 | ++ (C) |
| 77 | ++ (C) |
| 78 | ++ (B) |
| 79 | +++ (C) |
| 80 | +++ (C) |
| 81 | ++ (B) |
| 82 | ++ (B) |
| 83 | ++ (B) |
| 84 | + (C) |
| 85 | ++ (B) |
| 86 | ++ (B) |
| 87 | +++ (A) |
| 88 | +++ (A) |
| 89 | +++ (B) |
| 90 | ++ (C) |
| 91 | +++ (B) |
| 92 | +++ (B) |
| 93 | +++ (C) |
| 94 | +++ (C) |
| 95 | +++ (C) |
| 96 | +++ (C) |
| 97 | ++ (C) |
| 98 | +++ (B) |
| 99 | ++ (B) |
| 100 | +++ (C) |
| 101 | +++ (B) |
| 102 | ++ (C) |
| 103 | +++ (C) |
| 104 | ++ (C) |
| 105 | +++ (C) |
| 107 | +++ (C) |
| 108 | ++ (C) |
| 109 | ++ (C) |
| 111 | ++ (C) |
| 112 | ++ (C) |
| 113 | ++ (C) |
| 114 | ++ (C) |
| 115 | ++ (C) |
| 118 | ++ (C) |
| 121 | ++ (C) |
| 124 | ++ (C) |
| 126 | +++ (C) |
| 128 | ++ (C) |
| 131 | + (C) |
| 134 | ++ (C) |
| 138 | + (B) |
| 139 | ++ (C) |
| 140 | ++ (C) |
| 142 | ++ (C) |

Many of the compounds of Examples 1-143 were run in the recited assay and the results are reported in the following Table 5. In the following table:
“+” indicates that the compound showed activity of >1 μM in BT474 tumor cells;
“++” indicates that the compound showed activity of between 100 nM and 1 μM in BT474 tumor cells; and
“+++” indicates that the compound showed activity of less than 100 nM in BT474 tumor cells.

TABLE 5
Activity in BT474 Tumor Cells

| | Activity |
|---|---|
| Example | (Method) |
| 1 | +++ (A) |
| 2 | +++ (C) |
| 3 | +++ (A) |
| 4 | ++ (C) |
| 5 | +++ (C) |
| 6 | +++ (A) |
| 7 | +++ (B) |
| 8 | +++ (B) |
| 9 | +++ (B) |
| 10 | +++ (C) |
| 11 | +++ (C) |
| 12 | +++ (C) |
| 13 | +++ (C) |
| 14 | +++ (A) |
| 15 | +++ (B) |
| 16 | +++ (C) |
| 17 | +++ (C) |
| 18 | ++ (C) |
| 19 | +++ (B) |
| 20 | +++ (B) |
| 21 | +++ (A) |
| 22 | +++ (C) |
| 23 | +++ (C) |
| 24 | +++ (C) |
| 25 | ++ (C) |
| 26 | ++ (C) |
| 27 | +++ (C) |
| 28 | +++ (C) |
| 29 | +++ (C) |
| 30 | ++ (B) |
| 31 | ++ (C) |
| 32 | ++ (C) |
| 33 | +++ (C) |
| 34 | +++ (C) |
| 35 | + (C) |
| 36 | +++ (C) |
| 37 | ++ (C) |
| 38 | ++ (C) |
| 39 | +++ (C) |
| 40 | ++ (C) |
| 44 | ++ (C) |
| 45 | ++ (C) |
| 46 | ++ (C) |
| 47 | + (C) |
| 48 | + (C) |
| 49 | ++ (B) |
| 50 | ++ (A) |
| 51 | ++ (A) |
| 52 | +++ (A) |
| 53 | +++ (A) |
| 54 | +++ (A) |
| 55 | +++ (A) |
| 56 | +++ (C) |
| 57 | +++ (A) |
| 58 | +++ (A) |
| 59 | +++ (A) |
| 60 | ++ (A) |
| 61 | ++ (A) |
| 62 | ++ (A) |
| 63 | ++ (C) |
| 64 | ++ (B) |
| 65 | +++ (A) |
| 66 | +++ (A) |
| 67 | +++ (C) |
| 68 | +++ (C) |
| 69 | ++ (A) |
| 70 | ++ (A) |
| 71 | ++ (A) |
| 72 | + (C) |
| 73 | ++ (C) |
| 74 | ++ (C) |
| 75 | +++ (C) |
| 76 | +++ (C) |
| 77 | ++ (C) |
| 78 | ++ (B) |
| 79 | +++ (C) |
| 80 | +++ (C) |
| 81 | ++ (B) |
| 82 | ++ (B) |
| 83 | +++ (B) |
| 84 | + (C) |
| 85 | ++ (B) |
| 86 | ++ (B) |
| 87 | ++ (A) |
| 88 | ++ (A) |
| 89 | +++ (B) |
| 90 | +++ (C) |
| 91 | +++ (B) |
| 92 | +++ (B) |
| 93 | +++ (C) |
| 94 | +++ (C) |
| 95 | +++ (C) |
| 96 | ++ (C) |
| 97 | ++ (C) |
| 98 | +++ (B) |
| 99 | ++ (B) |
| 100 | +++ (C) |
| 101 | +++ (B) |
| 102 | ++ (C) |
| 103 | +++ (C) |
| 104 | +++ (C) |
| 105 | +++ (C) |
| 106 | + (C) |
| 107 | +++ (C) |
| 108 | +++ (C) |
| 109 | ++ (C) |
| 110 | ++ (C) |
| 111 | +++ (C) |
| 112 | +++ (C) |
| 113 | ++ (C) |
| 114 | ++ (C) |
| 115 | ++ (C) |
| 117 | ++ (C) |
| 118 | ++ (C) |
| 119 | ++ (C) |
| 120 | +++ (C) |
| 121 | ++ (C) |
| 122 | ++ (C) |
| 123 | ++ (C) |
| 124 | ++ (C) |
| 125 | ++ (C) |
| 126 | +++ (C) |
| 128 | ++ (C) |
| 129 | + (C) |
| 130 | + (C) |
| 131 | + (C) |
| 132 | ++ (C) |
| 133 | + (C) |
| 134 | ++ (C) |
| 135 | ++ (C) |
| 136 | ++ (C) |
| 137 | ++ (C) |
| 138 | + (B) |
| 139 | ++ (C) |
| 140 | ++ (C) |
| 142 | + (B) |

Many of the compounds of Examples 1-143 were run in the recited assay and the results are reported in the following Table 6. In the following table:
“+” indicates that the compound showed activity of >1 μM in Colo205 tumor cells;
“++” indicates that the compound showed activity of between 100 nM and 1 μM in Colo205 tumor cells; and
“+++” indicates that the compound showed activity of less than 100 nM in Colo205 tumor cells.

TABLE 6
Activity in Colo205 Tumor Cells

| | Activity |
|---|---|
| Example | (Method) |
| 1 | ++ (A) |
| 2 | +++ (C) |
| 3 | ++ (A) |
| 4 | ++ (C) |
| 5 | ++ (C) |
| 6 | ++ (A) |
| 7 | + (B) |
| 8 | ++ (B) |
| 9 | + (B) |
| 10 | ++ (C) |
| 11 | ++ (C) |
| 12 | ++ (C) |
| 13 | ++ (C) |
| 14 | +++ (A) |
| 15 | ++ (B) |
| 16 | +++ (A) |
| 17 | ++ (C) |
| 18 | + (C) |
| 19 | ++ (B) |
| 20 | + (B) |
| 21 | ++ (A) |
| 22 | ++ (C) |
| 23 | ++ (C) |
| 24 | ++ (C) |
| 25 | ++ (C) |
| 26 | ++ (C) |
| 27 | ++ (C) |
| 28 | ++ (C) |
| 29 | ++ (C) |
| 30 | + (B) |
| 31 | ++ (C) |
| 32 | ++ (C) |
| 33 | ++ (C) |
| 34 | ++ (C) |
| 35 | + (C) |
| 36 | ++ (C) |
| 37 | ++ (C) |
| 38 | ++ (C) |
| 39 | ++ (C) |
| 40 | ++ (C) |
| 41 | + (C) |
| 42 | + (C) |
| 43 | ++ (C) |
| 44 | ++ (C) |
| 45 | ++ (C) |
| 46 | ++ (C) |
| 47 | + (C) |
| 48 | + (C) |
| 49 | + (B) |
| 50 | + (A) |
| 51 | + (A) |
| 52 | ++ (A) |
| 53 | ++ (A) |
| 54 | + (A) |
| 55 | ++ (A) |
| 56 | ++ (C) |
| 57 | ++ (A) |
| 58 | ++ (A) |
| 59 | ++ (A) |
| 60 | ++ (A) |
| 61 | ++ (A) |
| 62 | ++ (A) |
| 63 | ++ (B) |
| 64 | + (B) |
| 65 | ++ (A) |
| 66 | ++ (A) |
| 67 | +++ (C) |
| 68 | ++ (C) |
| 69 | + (A) |
| 70 | ++ (A) |
| 71 | + (A) |
| 72 | + (C) |
| 73 | ++ (C) |
| 74 | ++ (C) |
| 75 | ++ (C) |
| 76 | ++ (C) |
| 77 | +++ (B) |
| 78 | ++ (B) |
| 79 | ++ (C) |
| 80 | + (C) |
| 81 | ++ (B) |
| 82 | ++ (B) |
| 83 | ++ (B) |
| 84 | + (C) |
| 85 | + (B) |
| 86 | ++ (B) |
| 87 | + (A) |
| 89 | ++ (B) |
| 90 | ++ (C) |
| 91 | ++ (B) |
| 92 | ++ (B) |
| 93 | ++ (C) |
| 94 | ++ (C) |
| 95 | + (C) |
| 96 | + (C) |
| 97 | + (C) |
| 98 | + (B) |
| 99 | + (B) |
| 100 | ++ (C) |
| 101 | + (B) |
| 102 | + (C) |
| 103 | + (B) |
| 104 | ++ (C) |
| 105 | ++ (C) |
| 106 | + (C) |
| 107 | ++ (C) |
| 108 | ++ (C) |
| 109 | + (C) |
| 110 | + (C) |
| 111 | ++ (C) |
| 112 | ++ (C) |
| 113 | + (C) |
| 114 | ++ (C) |
| 115 | ++ (C) |
| 116 | ++ (C) |
| 117 | + (C) |
| 118 | + (C) |
| 119 | ++ (C) |
| 120 | ++ (C) |
| 121 | ++ (C) |
| 122 | + (C) |
| 123 | + (C) |
| 124 | ++ (C) |
| 125 | ++ (C) |
| 126 | ++ (C) |
| 127 | + (C) |
| 128 | + (C) |
| 129 | ++ (C) |
| 130 | + (C) |
| 131 | + (C) |
| 132 | + (C) |
| 133 | ++ (C) |
| 134 | + (C) |
| 135 | + (C) |
| 136 | + (C) |
| 137 | + (C) |
| 138 | + (B) |
| 139 | ++ (C) |
| 140 | ++ (C) |
| 142 | + (B) |

Many of the compounds of Examples 1-143 were run in the recited assay and the results are reported in the following Table 7. In the following table:
“+” indicates that the compound showed activity of >1 μM in SK-MEL-28 tumor cells;
“++” indicates that the compound showed activity of between 100 nM and 1 μM in SK-MEL-28 tumor cells; and
“+++” indicates that the compound showed activity of less than 100 nM in SK-MEL-28 tumor cells.

TABLE 7
Activity in SK-MEL-28 Tumor Cells

| | Activity |
|---|---|
| Example | (Method) |
| 2 | ++ (C) |
| 4 | ++ (C) |
| 5 | ++ (C) |
| 10 | ++ (C) |
| 11 | +++ (C) |
| 12 | + (C) |
| 13 | + (C) |
| 17 | + (C) |
| 18 | + (C) |
| 22 | ++ (C) |
| 23 | + (C) |
| 24 | ++ (C) |
| 25 | ++ (C) |
| 26 | ++ (C) |
| 27 | ++ (C) |
| 28 | ++ (C) |
| 29 | + (C) |
| 31 | + (C) |
| 32 | ++ (C) |
| 33 | ++ (C) |
| 34 | ++ (C) |
| 35 | + (C) |
| 36 | + (C) |
| 37 | ++ (C) |
| 38 | ++ (C) |
| 39 | + (C) |
| 40 | ++ (C) |
| 41 | + (C) |
| 42 | + (C) |
| 43 | + (C) |
| 44 | ++ (C) |
| 45 | + (C) |
| 46 | + (C) |
| 47 | + (C) |
| 56 | + (C) |
| 63 | + (C) |
| 67 | ++ (C) |
| 68 | ++ (C) |
| 72 | + (C) |
| 73 | + (C) |
| 74 | + (C) |
| 75 | + (C) |
| 77 | + (C) |
| 79 | + (C) |
| 80 | ++ (C) |
| 84 | + (C) |
| 90 | + (C) |
| 93 | ++ (C) |
| 94 | + (C) |
| 95 | + (C) |
| 96 | + (C) |
| 97 | + (C) |
| 103 | + (C) |
| 104 | + (C) |
| 105 | + (C) |
| 106 | + (C) |
| 107 | ++ (C) |
| 108 | + (C) |
| 109 | + (C) |
| 110 | + (C) |
| 111 | ++ (C) |
| 112 | + (C) |
| 113 | + (C) |
| 114 | ++ (C) |
| 115 | + (C) |
| 116 | + (C) |
| 117 | + (C) |
| 118 | + (C) |
| 119 | + (C) |
| 120 | + (C) |
| 121 | ++ (C) |
| 122 | + (C) |
| 123 | + (C) |
| 124 | + (C) |
| 125 | ++ (C) |
| 126 | ++ (C) |
| 127 | + (C) |
| 128 | + (C) |
| 129 | + (C) |
| 130 | + (C) |
| 131 | + (C) |
| 132 | + (C) |
| 133 | ++ (C) |
| 134 | + (C) |
| 135 | + (C) |
| 136 | + (C) |
| 137 | + (C) |
| 140 | ++ (C) |
| 142 | + (C) |

Many of the compounds of Examples 1-143 were run in the recited assay and the results are reported in the following Table 8. In the following table:
“+” indicates that the compound showed activity of >1 μM in A375P F11s tumor cells;
“++” indicates that the compound showed activity of between 100 nM and 1 μLM in A375P F11s tumor cells; and
“+++” indicates that the compound showed activity of less than 100 nM in A375P F11s tumor cells.

TABLE 8
Activity in A375P F11s Tumor Cells

| | Activity |
|---|---|
| Example | (Method) |
| 2 | ++ (C) |
| 4 | ++ (C) |
| 5 | ++ (C) |
| 10 | ++ (C) |
| 11 | ++ (C) |
| 12 | ++ (C) |
| 13 | ++ (C) |
| 17 | ++ (C) |
| 22 | ++ (C) |
| 23 | ++ (C) |
| 24 | ++ (C) |
| 25 | ++ (C) |
| 26 | ++ (C) |
| 27 | ++ (C) |
| 28 | ++ (C) |
| 31 | ++ (C) |
| 32 | ++ (C) |
| 33 | ++ (C) |
| 34 | ++ (C) |
| 36 | + (C) |
| 37 | ++ (C) |
| 38 | ++ (C) |
| 39 | ++ (C) |
| 40 | ++ (C) |
| 44 | + (C) |
| 45 | ++ (C) |
| 56 | ++ (C) |
| 63 | ++ (C) |
| 67 | ++ (C) |
| 68 | ++ (C) |
| 72 | + (C) |
| 74 | ++ (C) |
| 75 | ++ (C) |
| 77 | ++ (C) |
| 79 | ++ (C) |
| 80 | ++ (C) |
| 84 | + (C) |
| 90 | ++ (C) |
| 93 | ++ (C) |
| 94 | + (C) |
| 95 | ++ (C) |
| 104 | ++ (C) |
| 105 | ++ (C) |
| 107 | ++ (C) |
| 108 | ++ (C) |
| 109 | + (C) |
| 111 | ++ (C) |
| 112 | ++ (C) |
| 113 | ++ (C) |
| 114 | ++ (C) |
| 115 | + (C) |
| 118 | + (C) |
| 121 | ++ (C) |
| 124 | + (C) |
| 126 | ++ (C) |
| 128 | + (C) |
| 131 | + (C) |
| 134 | + (C) |

Many of the compounds of Examples 1-143 were run in the recited assay and the results are reported in the following Table 9. In the following table:
“+” indicates that the compound showed activity of >1 μM in HT-29 tumor cells;
“++” indicates that the compound showed activity of between 100 nM and 1 μM in HT-29 tumor cells; and
“+++” indicates that the compound showed activity of less than 100 nM in HT-29 tumor cells.

TABLE 9
Activity in HT-29 Tumor Cells

| | Activity |
|---|---|
| Example | (Method) |
| 2 | ++ (C) |
| 4 | ++ (C) |
| 5 | ++ (C) |
| 10 | ++ (C) |
| 11 | ++ (C) |
| 12 | ++ (C) |
| 13 | ++ (C) |
| 16 | ++ (C) |
| 17 | ++ (C) |
| 22 | ++ (C) |
| 23 | ++ (C) |
| 24 | ++ (C) |
| 25 | ++ (C) |
| 26 | ++ (C) |
| 27 | ++ (C) |
| 28 | ++ (C) |
| 31 | ++ (C) |
| 32 | ++ (C) |
| 33 | ++ (C) |
| 34 | ++ (C) |
| 36 | ++ (C) |
| 37 | ++ (C) |
| 38 | ++ (C) |
| 39 | ++ (C) |
| 40 | ++ (C) |
| 44 | ++ (C) |
| 45 | ++ (C) |
| 56 | ++ (C) |
| 63 | ++ (C) |
| 67 | ++ (C) |
| 68 | ++ (C) |
| 72 | ++ (C) |
| 74 | ++ (C) |
| 75 | ++ (C) |
| 77 | ++ (C) |
| 79 | ++ (C) |
| 80 | ++ (C) |
| 84 | + (C) |
| 90 | ++ (C) |
| 93 | ++ (C) |
| 94 | ++ (C) |
| 95 | ++ (C) |
| 104 | ++ (C) |
| 105 | ++ (C) |
| 107 | ++ (C) |
| 108 | ++ (C) |
| 109 | + (C) |
| 111 | ++ (C) |
| 112 | ++ (C) |
| 113 | ++ (C) |
| 114 | ++ (C) |
| 115 | ++ (C) |
| 118 | + (C) |
| 121 | ++ (C) |
| 124 | + (C) |
| 126 | ++ (C) |
| 128 | ++ (C) |
| 131 | + (C) |
| 134 | ++ (C) |

Many of the compounds of Examples 1-143 were run in the recited assay and the results are reported in the following Table 10. In the following table:
“+” indicates that the compound showed activity of >1 μM in SK-MEL-3 tumor cells;
“++” indicates that the compound showed activity of between 100 nM and 1 μM in SK-MEL-3 tumor cells; and
“+++” indicates that the compound showed activity of less than 100 nM in SK-MEL-3 tumor cells.

TABLE 10
Activity in SK-MEL-3 Tumor Cells

| | Activity |
|---|---|
| Example | (Method) |
| 2 | ++ (C) |
| 4 | ++ (C) |
| 5 | ++ (C) |
| 10 | ++ (C) |
| 11 | ++ (C) |
| 12 | ++ (C) |
| 13 | ++ (C) |
| 17 | + (C) |
| 22 | ++ (C) |
| 23 | ++ (C) |
| 24 | ++ (C) |
| 25 | ++ (C) |
| 26 | ++ (C) |
| 27 | ++ (C) |
| 28 | ++ (C) |
| 31 | ++ (C) |
| 32 | ++ (C) |
| 33 | ++ (C) |
| 34 | ++ (C) |
| 36 | + (C) |
| 37 | + (C) |
| 38 | ++ (C) |
| 39 | ++ (C) |
| 40 | ++ (C) |
| 44 | + (C) |
| 45 | ++ (C) |
| 56 | + (C) |
| 63 | ++ (C) |
| 67 | ++ (C) |
| 68 | ++ (C) |
| 72 | + (C) |
| 74 | + (C) |
| 75 | ++ (C) |
| 77 | +++ (C) |
| 79 | + (C) |
| 80 | ++ (C) |
| 84 | + (C) |
| 90 | + (C) |
| 93 | ++ (C) |
| 94 | ++ (C) |
| 95 | + (C) |
| 104 | ++ (C) |
| 105 | + (C) |
| 107 | ++ (C) |
| 108 | + (C) |
| 109 | + (C) |
| 111 | ++ (C) |
| 112 | + (C) |
| 113 | ++ (C) |
| 114 | + (C) |
| 115 | ++ (C) |
| 118 | + (C) |
| 121 | ++ (C) |
| 124 | + (C) |
| 126 | ++ (C) |
| 128 | + (C) |
| 131 | + (C) |
| 134 | + (C) |

### Pharmaceutical Formulation Example—Preparation of Capsules Containing a Compound of the Invention (Freebase):

Contents in each capsule:
   =60 mg Active Pharmaceutical ingredient (API)+60 mg Avicel+13 mg SSG.
133 mg total powder in a size 0 hard gelatin capsule. The Avicel/SSG weight may be reasonably approximate.

### Procedure:

1. Separate the halves of hard-gelatin capsule and mark/identify each as appropriate/needed.
2. Place the bottom capsule half in capsule filler with the filling funnel on top.
3. Weigh the components (Avicel, Sodium Starch Glycolate (SSG), API) onto a single weigh paper (tared on an analytical balance between each weighing).
4. Record weights of each component.
5. Carefully and thoroughly mix the dry powders on the weigh paper with a small spatula.
6. Carefully transfer the mixed powders into the capsule through the funnel.
7. Place the top half onto the capsule and close until secure, shake capsule to mix/distribute contents.
8. IF powder begins to near top of capsule, gently tap capsule and powder should settle.
9. Place the capsule into a small appropriately labeled bottle (but large enough to easily remove it).

### Pharmaceutical Formulation Example—Preparation of Tablets Containing a Compound of the Invention (Freebase):

| | Quantity | % |
|---|---|---|
| Component | (mg/tablet) | w/w |
| Core Tablet | | |
| API | 405.0 | 71.6 |
| Lactose monohydrate | 59.0 | 10.4 |
| Polysorbate 80 | 1.0 | 0.2 |
| Povidone | 40.0 | 7.1 |
| Colloidal Silicon Dioxide | 5.5 | 1.0 |
| Crospovidone | 51.0 | 9.0 |
| Magnesium Stearate | 4.5 | 0.8 |
| Purified Water | qs | |
| Film Coating | | |
| Opadry ® Orange, YS-1-13065-A | 17.0 | 3.0 |
| Purified water | qs | |

### Procedure:

1. Sieve Lactose, Silicon dioxide, Crospovidone and half Povidone.

### 2. Add API.

3. Granulate in High Shear Granulator with granulating solution containing dissolved Polysorbate 80 and other half of Povidone in Purified water.
4. Mill using Comil 197, 0.375″ screen.
5. Dry using Fluid Bed Dryer
6. Mill using Comil 197, 0.075″ screen
7. Add Crospofidone, magnesium stearate.
8. Blend 5 minute
9. Compress tablet
10. Aqueous film coat tablet

What is claimed is:

1. A compound of formula (I): 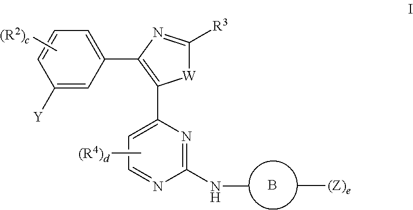
wherein:
Y is a moiety selected from i, ii, and iii: 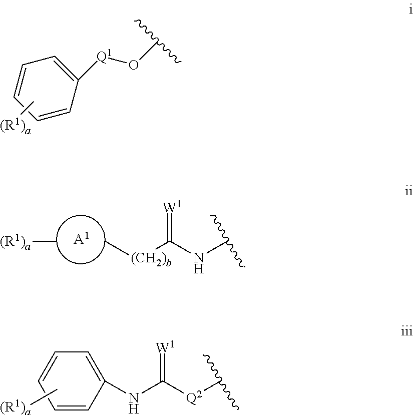
wherein:
a is 0, 1, 2 or 3;
each R¹ is the same or different and is independently selected from halo, alkyl, haloalkyl, —OR⁶, —R⁵—OR⁶, —C(O)R⁶, —CO₂R⁶, —S(O)_{f}R⁶, —R⁵—S(O)_{f}R⁶, —NR⁶R⁷, —R⁵—NR⁶R⁷, —CN and —R⁵—CN;
f is 0, 1 or 2;
Q¹ is —CH₂— or —SO₂—;
Ring A¹ is cycloalkyl, phenyl or 5-10 membered heteroaryl having 1 or 2 heteroatoms selected from N, O and S;
b is 0 or 1;
W¹ is O or S;
Q² is a bond or —N(H)—;
c is 0, 1 or 2;
each R² is the same or different and is independently selected from halo, alkyl, haloalkyl, —OR⁶, —S(O)_{f}R⁶, —NR⁶R⁷, —CN and —NO₂;
W is —O— or —S—;
R³ is selected from H, alkyl, haloalkyl, alkenyl, cycloalkyl, —R⁵-cycloalkyl, Ph, Het,
—R⁵—OR⁶, —R⁵—S(O)_{f}R⁶, —R⁵—S(O)₂—NR⁶R⁷, —NR⁶R⁷, —N(R⁶)-cycloalkyl, —N(R⁶)Ph,
—N(R⁶)Het, —N(R⁶)R⁵—Het, —N(R⁶)—R⁵—OR⁷, —N(R⁶)—R⁵—NR⁶R⁷, —N(H)C(O)R⁶,
—R⁵—N(H)C(O)R⁶, —N(R⁶)—C(O)—NR⁶R⁷, —N(H)SO₂R⁶, —N(R⁶)—R⁵—S(O)_{f}R⁷, and
—N(R⁶)—S(O)₂—NR⁶R⁷,
wherein each of said cycloalkyl is optionally substituted with 1 or 2 substituents which are the same or different and are each independently selected from halo, C₁₋₃alkyl, haloalkyl, OH, O—C₁₋₃alkyl, oxo, S(C₁₋₃alkyl), SO₂, NH₂, N(H)C₁₋₃alkyl, and N(C₁₋₃alkyl)₂;
d is 0, 1 or 2;
each R⁴ is the same or different and is independently selected from halo, alkyl, haloalkyl, —S(O)_{f}R⁶, —NR⁶R⁷, —CN and —NO₂;
each Ph is the same or different and is independently phenyl optionally substituted with 1, 2 or 3 substituents which are the same or different and are each independently selected from halo, C₁₋₃alkyl, O—C₁₋₃alkyl, C₁₋₃alkylene-O—C₁₋₃alkyl, OH, C₁₋₃alkylene-OH, NH₂, N(H)C₁₋₃alkyl, N(C₁₋₃alkyl)₂, CN and NO₂;
each Het is the same or different and is independently selected from 4-6 membered heterocycle having 1 or 2 heteroatoms selected from N, O and S and optionally substituted with 1, 2 or 3 substituents which are the same or different and are each independently selected from halo, C₁₋₃alkyl, O—C₁₋₃alkyl,
C₁₋₃alkylene-O—C₁₋₃alkyl, OH, oxo, C(O)(C₁₋₃alkyl), C(O)NH₂, C(O)N(C₁₋₃alkyl)₂, SO₃(H), SO₂(C₁₋₃alkyl), C₁₋₃alkylene-SO₃(H),
C₁₋₃alkylene-SO₂(C₁₋₃alkyl), NH₂, N(H)C₁₋₃alkyl, N(C₁₋₃alkyl)₂, CN, —CH₂CN, and NO₂;
each R⁵ is the same or different and is independently C₁₋₄alkylene;
Ring B is selected from phenyl, 9-10 membered aryl, 5-6 membered heteroaryl, and 9-10 membered heteroaryl, each heteroaryl having 1, 2 or 3 heteroatoms selected from N, O and S;
wherein when Ring B is selected from phenyl and 5-6 membered heteroaryl, then
e is 0, 1, 2 or 3; and
each Z is the same or different and is independently selected from:
halo, alkyl, haloalkyl, alkenyl,
Het², —R⁵Het², Het³-Het²,
oxo, —OR⁶, —R⁵—OR⁶, —O—R⁵—OR⁶, —OHet², —O—R⁵—Het², —O—R⁵—NR⁶R⁷,
—O—R⁵—S(O)₂R⁶, —C(O)NR⁶R⁷, —R⁵—C(O)NR⁶R⁷, —CO₂R⁶, —R⁵—CO₂R⁶,
—S(O)_{f}R⁶, —R⁵—S(O)₂R⁶, —S(O)_{f}Het², —R⁵—S(O)₂Het², —S(O)₂NR⁶R⁷, —R⁵—S(O)₂NR⁶R⁷, —S(O)₂—R⁵—NR⁶R⁷,
—NR⁶R⁷, —R⁵—NR⁶R⁷, —N(R⁶)Het², —N(R⁶)—R⁵cycloalkyl, —N(R⁶)—R⁵—Het²,
—N(R⁶)—R⁵—OR⁷, —N(R⁶)—R⁵—S(O)_{f}R⁷, —N(R⁶)—R⁵—CN,
—N(R⁶)—R⁵—NR⁶R⁷, —N(H)S(O)₂R⁶, —N(R⁶)—C(O)—NR⁶R⁷,
—N(R⁶)—S(O)₂—NR⁶R⁷,
—CN, —R⁵—CN and —NO₂; and
when Ring B is a 9-10 membered aryl or 9-10 membered heteroaryl, then
e is 0, 1 or 2 and
each Z is the same or different and is independently selected from halo, alkyl, oxo, —OR⁶ and —NR⁶R⁷;
each Het² is the same or different and is independently heterocycle or heteroaryl, said heterocycle or heteroaryl having 1 or 2 heteroatoms selected from N, O and S and each optionally substituted with 1, 2 or 3 substituents which are the same or different and are each independently selected from:
halo, C₁₋₃alkyl, haloC₁₋₃alkyl, O—C₁₋₃alkyl, C₁₋₃alkylene-O—C₁₋₃alkyl, OH, C₁₋₃alkylene-OH, oxo, C(O)(C₁₋₃alkyl), C(O)₂—C₁₋₃alkyl,
C(O)—(C₁₋₃alkylene)-O(C₁₋₃alkyl), C(O)₂-benzyl, SO₃H, SO₂(C₁₋₃alkyl), C₁₋₃alkylene-SO₃H, C₁₋₃alkylene-SO₂(C₁₋₃alkyl), NH₂, N(H)C₁₋₃alkyl, N(C₁₋₃alkyl)₂,
CN and C₁₋₃alkylene-CN;
Het³ is selected from 4-7 membered heterocycle and 5-7 membered heteroaryl, said heterocycle or heteroaryl having 1 or 2 heteroatoms selected from N, O and S and optionally substituted with 1 or 2 additional substituents which are the same or different and are each independently selected from halo, C₁₋₃alkyl,
haloC₁₋₃alkyl, and O—C₁₋₃alkyl;
each R⁶ and each R⁷ is the same or different and is independently H, alkyl or haloalkyl;
or a pharmaceutically acceptable salt thereof.

2. The compound according to claim 1, wherein Y is moiety i.

3. (canceled)

4. (canceled)

5. (canceled)

6. The compound according to claim 1, wherein Y is moiety ii.

7. (canceled)

8. (canceled)

9. (canceled)

10. (canceled)

11. (canceled)

12. (canceled)

13. The compound according to claim 1, wherein Y is moiety iii.

14. (canceled)

15. (canceled)

16. (canceled)

17. (canceled)

18. (canceled)

19. The compound according to claim 1, wherein W is S.

20. (canceled)

21. (canceled)

22. (canceled)

23. (canceled)

24. (canceled)

25. (canceled)

26. (canceled)

27. (canceled)

28. (canceled)

29. (canceled)

30. (canceled)

31. (canceled)

32. (canceled)

33. A compound selected from:
N-{3-[2-Amino-5-(2-{[3-{[2-(dimethylamino)ethyl]oxy}-4-(methyloxy)phenyl]amino}-4-pyrimidinyl)-1,3-thiazol-4-yl]phenyl}-2,6-difluoro-N-methylbenzamide;
N-(3-{2-Amino-5-[2-({3-chloro-4-[2-(dimethylamino)ethoxy]-phenyl}amino)pyrimidin-4-yl]-1,3-thiazol-4-yl}phenyl)-2,6-difluorobenzamide-formic acid
N-[3-(2-Amino-5-{2-[(3,4,5-trimethoxyphenyl)amino]pyrimidiN-4-yl}-1,3-thiazol-4-yl)phenyl]-2,6-difluorobenzamide;
N-[3-(2-(Dimethylamino)-5-{2-[(4-{[2-(dimethylamino)ethyl]oxy}phenyl)amino]-4-pyrimidinyl}-1,3-thiazol-4-yl)phenyl]-2,6-difluorobenzamide;
2,6-Difluoro-N-{3-[5-(2-{[4-(methyloxy)-3-(4-methyl-1-piperazinyl)phenyl]amino}-4-pyrimidinyl)-1,3-thiazol-4-yl]phenyl}benzamide;
N-[3-(5-{2-[(4-{[2-(Dimethylamino)ethyl]oxy}-3-fluorophenyl)amino]-4-pyrimidinyl}-2-ethyl-1,3-thiazol-4-yl)phenyl]-2,6-difluorobenzamide; and
N-[3-(5-{2-[(3-Chloro-4-{[2-(1-pyrrolidinyl)ethyl]oxy}phenyl)amino]-4-pyrimidinyl}-2-ethyl-1,3-thiazol-4-yl)phenyl]-2,6-difluorobenzamide;
and pharmaceutically acceptable salts thereof.

34. A pharmaceutical composition comprising a compound according to claim 1 and a pharmaceutically acceptable carrier, diluent or excipient.

35. The pharmaceutical composition according to claim 34 further comprising a chemotherapeutic agent.

36. A method for treating a susceptible neoplasm selected from breast cancer, colorectal cancer, melanoma, non-small cell lung cancer, ovarian cancer, and thyroid cancer in a mammal in need thereof, said method comprising administering to the mammal a therapeutically effective amount of a compound according to claim 1.

37. (canceled)

38. (canceled)

39. A process for preparing a compound according to claim 1, wherein all variables are as defined in claim 1, said process comprising reacting a compound of formula (VIII), (VIII-A), (VIII-B) or (XXVI): 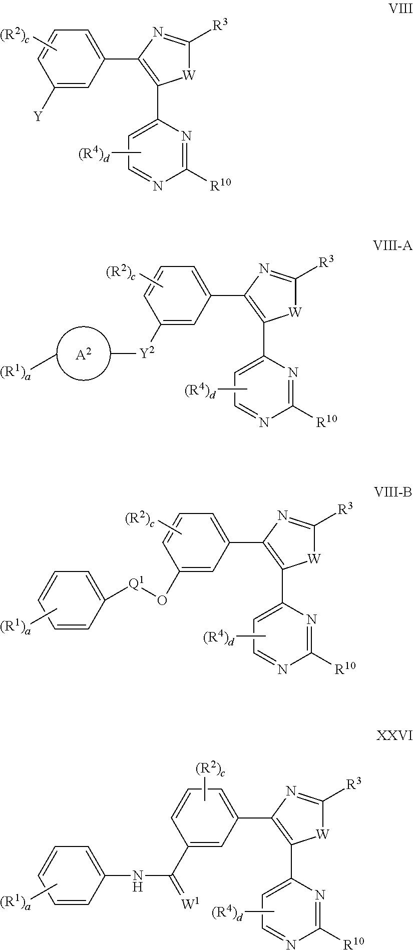
wherein R¹⁰ is halo or thiomethyl;
Y in formula (VIII) is moiety ii or moiety iii wherein Q² is —N(H)—;
Y² is —C(O)NH, —CH₂—C(O)NH—, or —N(H)C(O)N(H)—;
Ring A² is phenyl or Ring A¹;
and all other variables are as defined in claim 1;
with an aniline of formula (IX): 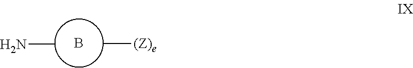
to prepare a compound of formula (I).

40. A process for preparing a compound according to claim 1, wherein Y is moiety ii or moiety iii wherein Q² is —N(H)—, and all other variables are as defined in claim 1, said process comprising reacting a compound of formula (XIV): 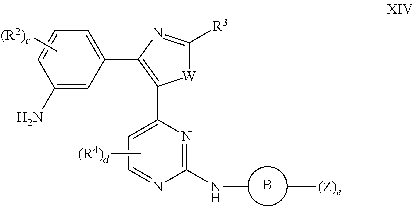
with a compound of formula (VII-A): 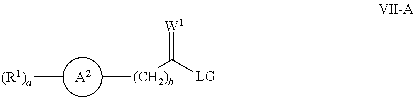
wherein Ring A² is phenyl or Ring A¹ and LG is a suitable leaving group; or a compound of formula (VII-B): 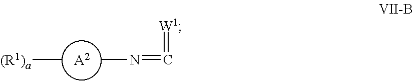
to prepare a compound of formula (I).

41. A process for preparing a compound according to claim 1, said process comprising reacting a compound of formula (XXXI): 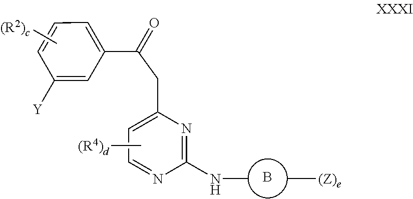
with a suitable brominating agent followed by reaction with one of:
i) a thiourea,
ii) a formamide,
iii) an amide,
iv) a thioamide, or
v) a urea;
to prepare a compound of formula (I).

42. A compound according to claim 1 for use in therapy.

43. A compound according to claim 1 for use in the treatment of a susceptible neoplasm selected from breast cancer, colorectal cancer, melanoma, non-small cell lung cancer, ovarian cancer, and thyroid cancer in a mammal.

44. (canceled)

45. A pharmaceutical composition comprising a compound according to claim 1 for use in the treatment of a susceptible neoplasm selected from breast cancer, colorectal cancer, melanoma, non-small cell lung cancer, ovarian cancer, and thyroid cancer in a mammal in need thereof.
